(19) United States
(12) Patent Application Publication
(10) Pub. No.: US 2026/0337146 A1
(43) Pub. Date: Oct. 29, 2026

(54) ORGANIC ELECTROLUMINESCENT COMPOUND AND ORGANIC ELECTROLUMINESCENT DEVICE COMPRISING THE SAME

(71) Applicant: DuPont Specialty Materials Korea Ltd., Chungcheongnam-do (KR)

(72) Inventors: Hyo-Soon PARK, Gyeonggi-do (KR); Hyun-Woo KANG, Gyeonggi-do (KR); Jin-Man KIM, Gyeonggi-do (KR); Mi-Ja LEE, Gyeonggi-do (KR); Samuel KIM, Gyeonggi-do (KR); Hong-Se OH, Gyeonggi-do (KR); Hyun KIM, Gyeonggi-do (KR); Han-Kyu YOO, Gyeonggi-do (KR)

(21) Appl. No.: 19/176,267

(22) Filed: Apr. 11, 2025

(30) Foreign Application Priority Data
May 2, 2024 (KR) 10-2024-0058427
Mar. 31, 2025 (KR) 10-2025-0040973

(51) Int. Cl. C07C 211/61; C09K 11/06; H10K 50/11; H10K 50/15; H10K 50/18; H10K 85/60

(52) U.S. Cl. CPC C07C 211/61; C09K 11/06; H10K 85/633; C07C 2603/26; C07C 2603/74; C09K 2211/1007; C09K 2211/1011; H10K 50/11; H10K 50/156; H10K 50/181; H10K 85/615

(57) ABSTRACT

The present disclosure relates to an organic electroluminescent compound and an organic electroluminescent device comprising the same. By comprising the organic electroluminescent compounds according to the present disclosure, an organic electroluminescent device with low driving voltage and/or high efficiency characteristics compared to a conventional organic electroluminescent device can be provided.

### TECHNICAL FIELD

The present disclosure relates to an organic electroluminescent compound and an organic electroluminescent device comprising the same.

### BACKGROUND ART

The TPD/Alq₃ bilayer small molecule organic electroluminescent device (OLED) with green-emission, which is constituted with a light-emitting layer and a charge transport layer, was first developed by Tang et al. of Eastman Kodak in 1987. Thereafter, studies on OLEDs have been rapidly effected, and OLEDs have been commercialized. At present, an organic electroluminescent device mainly includes phosphorescent materials having excellent luminous efficiency in panel realization. Accordingly, for prolonged use and high display resolution, an OLED having high luminous efficiency is necessary.

U.S. Pat. No. 8,129,037 B2 and Chinese Patent CN 110105225 B disclose a dihydrophenanthrene derivative as a hole transport material. However, said references do not disclose an organic electroluminescent compound with improved performance, such as low driving voltage and/or high efficiency characteristics, by comprising a dihydrophenanthrene compound having a specific substituent as an organic electroluminescent material as described in the present disclosure.

### DISCLOSURE OF THE INVENTION

### Problems to be Solved

The object of the present disclosure is, firstly, to provide an organic electroluminescent compound effective for producing an organic electroluminescent device having low driving voltage and/or high efficiency characteristics, and secondly, to provide an organic electroluminescent device comprising an organic electroluminescent compound according to the present disclosure.

### Solution to Problems

As a result of intensive studies to solve the technical problem above, the present inventors found that the aforementioned objective can be achieved by an organic electroluminescent compound represented by the following Formula 1 and an organic electroluminescent device comprising the same, thereby completing the present invention.

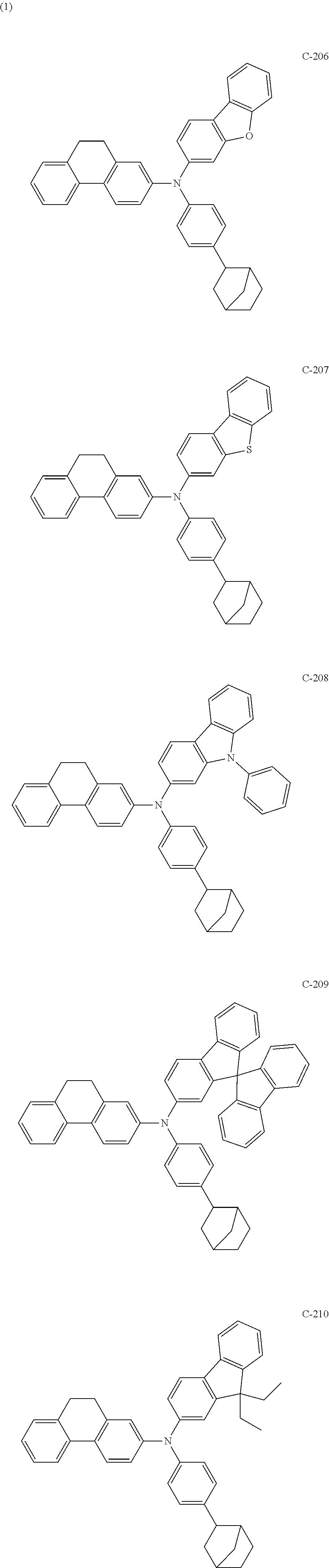 [SMILES: c1ccc2c(c1)CCc1cc(N(c3ccc(C4CC5CCC4C5)cc3)c3ccc4c(c3)oc3ccccc34)ccc1-2 ; c1ccc2c(c1)CCc1cc(N(c3ccc(C4CC5CCC4C5)cc3)c3ccc4c(c3)sc3ccccc34)ccc1-2 ; c1ccc(-n2c3ccccc3c3ccc(N(c4ccc(C5CC6CCC5C6)cc4)c4ccc5c(c4)CCc4ccccc4-5)cc32)cc1 ; c1ccc2c(c1)CCc1cc(N(c3ccc(C4CC5CCC4C5)cc3)c3ccc4c(c3)C3(c5ccccc5-c5ccccc53)c3ccccc3-4)ccc1-2 ; CCC1(CC)c2ccccc2-c2ccc(N(c3ccc(C4CC5CCC4C5)cc3)c3ccc4c(c3)CCc3ccccc3-4)cc21]

In Formula 1,
ring A and ring B each independently represent a substituted or unsubstituted (C₆-C₃₀)arene, or a substituted or unsubstituted (3- to 30-membered)heteroarene;
R₁ to R₄, Rₐ, and R_{b} each independently represent hydrogen, deuterium, halogen, cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring of (C₃-C₃₀) aliphatic ring and (C₆-C₃₀) aromatic ring, a substituted or unsubstituted mono- or di(C₁-C₃₀)alkylamino, a substituted or unsubstituted mono- or di(C₆-C₃₀)arylamino, a substituted or unsubstituted (C₁-C₃₀)alkyl(C₆-C₃₀)arylamino, a substituted or unsubstituted mono- or di(3-to 30-membered)heteroarylamino, or a substituted or unsubstituted (C₆-C₃₀)aryl(3- to 30-membered)heteroarylamino; or may be linked to the adjacent substituents to form a ring(s);
provided that at least one of R₁ to R₄, Rₐ, and R_{b} is represented by the following Formula 1-1;

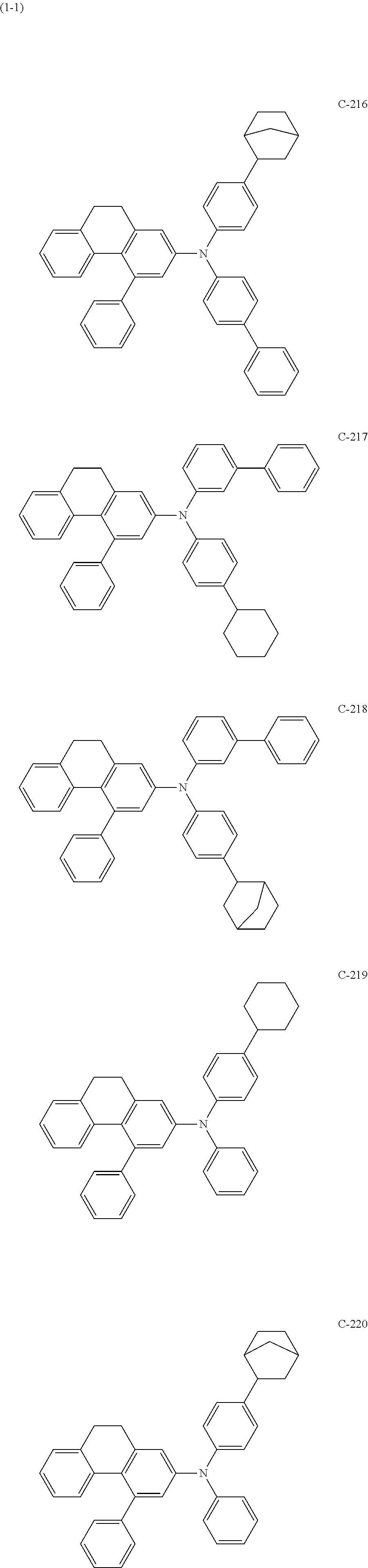 [SMILES: c1ccc(-c2ccc(N(c3ccc(C4CC5CCC4C5)cc3)c3cc4c(c(-c5ccccc5)c3)-c3ccccc3CC4)cc2)cc1 ; c1ccc(-c2cccc(N(c3ccc(C4CCCCC4)cc3)c3cc4c(c(-c5ccccc5)c3)-c3ccccc3CC4)c2)cc1 ; c1ccc(-c2cccc(N(c3ccc(C4CC5CCC4C5)cc3)c3cc4c(c(-c5ccccc5)c3)-c3ccccc3CC4)c2)cc1 ; c1ccc(-c2cc(N(c3ccccc3)c3ccc(C4CCCCC4)cc3)cc3c2-c2ccccc2CC3)cc1 ; c1ccc(-c2cc(N(c3ccccc3)c3ccc(C4CC5CCC4C5)cc3)cc3c2-c2ccccc2CC3)cc1]

In Formula 1-1,
L₁ to L₃ each independently represent a single bond, a substituted or unsubstituted (C₆-C₃₀)arylene, or a substituted or unsubstituted (3- to 30-membered)heteroarylene;
Ar₁ and Ar₂ each independently represent a substituted or unsubstituted (C₆-C₃₀)aryl, a substituted or unsubstituted (3- to 30-membered)heteroaryl, or a substituted or unsubstituted (C₃-C₃₀)cycloalkyl;
provided that at least one of Ar₁ and Ar₂ is a substituted or unsubstituted (C₃—C₃₀)cycloalkyl; and
n and m are integers of 1 to 8, and when n and m are integers greater than or equal to 2, each of Rₐ and each of R_{b} may be the same or different.

### Advantageous Effects of Invention

By comprising an organic electroluminescent compound according to the present disclosure, an organic electroluminescent device having low driving voltage and/or high efficiency characteristics can be provided.

### EMBODIMENTS OF THE INVENTION

Hereinafter, the present disclosure will be described in detail. However, the following description is intended to explain the invention, and is not meant in any way to restrict the scope of the invention.

The present disclosure relates to an organic electroluminescent compound represented by Formula 1, and an organic electroluminescent device comprising the organic electroluminescent compound.

The term “organic electroluminescent compound” in the present disclosure means a compound that may be used in an organic electroluminescent device, and may be comprised in any material layer constituting an organic electroluminescent device, as necessary.

Herein, the term “organic electroluminescent material” means a material that may be used in an organic electroluminescent device, and may comprise at least one compound.

The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron-blocking material, a light-emitting material (containing host and dopant materials), an electron buffer material, a hole-blocking material, an electron transport material, or an electron injection material, etc.

Herein, “(C₁-C₃₀)alkyl” is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, tert-butyl, etc. Herein, the “(C₃-C₃₀)cycloalkyl” is a saturated or partially unsaturated cyclic hydrocarbon substituent of single- or multi-ring, and is meant to be a mono- or polycyclic hydrocarbon having 3 to 30 ring skeleton carbon atoms, in which the number of carbon atoms is preferably 3 to 20, and more preferably 3 to 7. The single-ring cycloalkyl includes cyclopropyl, cyclobutyl, cyclopentyl, cyclohexyl, cyclohexenyl, cyclohexadienyl, cycloheptyl, cycloheptatrienyl, and cyclooctyl, etc. as non-limiting examples. The multi-ring cycloalkyl includes a spiro-ring, fused-ring, and crosslinked-ring cycloalkyl. The above cycloalkyl ring may be fused into an aryl, heteroaryl or heterocycloalkyl ring, and the cycloalkyl ring includes indanyl, tetrahydronaphthalenyl, benzocycloheptenyl, etc. as non-limiting examples. The cycloalkyl of crosslinked ring is meant to be a 5- to 20-membered, preferably 6- to 14-membered, and more preferably 7- to 10-membered all-carbon multi-ring group having any two rings sharing two carbon atoms that are not directly connected, which may include one or more double bonds, but none of the rings has a fully conjugated rr-electron system. Depending on the number of rings formed, it can be classified as bicyclic, tricyclic, tetracyclic, or polycyclic crosslinked cycloalkyl, preferably bicyclic, tricyclic, or tetracyclic, and more preferably bicyclic or tricyclic. The cycloalkyl of crosslinked ring includes, for example, adamantanyl, norbornanyl, norbornenyl, bicyclo[1.1.1]pentyl, bicyclo[2.1.1]hexyl, bicyclo[2.2.1]heptanyl, bicyclo[2.2.2]octyl, etc. Herein, the “(3- to 20-membered)heterocycloalkyl” is meant to be a saturated or partially unsaturated single- or multi-ring cyclic hydrocarbon substituent having a ring skeleton of 3 to 20 atoms, in which the number of carbon atoms is 3 to 7, preferably 5 to 7, and includes at least one heteroatom selected from the group consisting of B, N, O, S, Si, and P, preferably the group consisting of O, S, and N. For example, the above heterocycloalkyl includes tetrahydrofuran, pyrrolidine, thiolane, tetrahydropyran, etc. Herein, “(C₆-C₃₀)aryl(ene)” is meant to be a monocyclic or fused ring system radical derived from an aromatic hydrocarbon having 6 to 30 ring skeleton carbon atoms, which may be partially saturated, and may include a spiro structure. The number of carbon atoms in the ring skeleton is preferably 6 to 20, and more preferably 6 to 15. Examples of the aryl specifically may be phenyl, biphenyl, terphenyl, quaterphenyl, naphthyl, binaphthyl, phenylnaphthyl, naphthylphenyl, fluorenyl, phenylfluorenyl, dimethylfluorenyl, diphenylfluorenyl, benzofluorenyl, diphenylbenzofluorenyl, dibenzofluorenyl, phenanthrenyl, benzophenanthrenyl, phenylphenanthrenyl, anthracenyl, benzanthracenyl, indenyl, triphenylenyl, pyrenyl, tetracenyl, perylenyl, chrysenyl, benzochrysenyl, naphthacenyl, fluoranthenyl, benzofluoranthenyl, tolyl, xylyl, mesityl, cumenyl, spiro[fluoren-fluoren]yl(spirobifluorenyl), spiro[fluoren-benzofluoren]yl, azulenyl, etc. More specifically, the aryl may be o-tolyl, m-tolyl, p-tolyl, 2,3-xylyl, 3,4-xylyl, 2,5-xylyl, mesityl, o-cumenyl, m-cumenyl, p-cumenyl, p-t-butylphenyl, p-(2-phenylpropyl)phenyl, 4′-methylbiphenyl, 4″-t-butyl-p-terphenyl-4-yl, o-biphenyl, m-biphenyl, p-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 1-naphthyl, 2-naphthyl, 1-fluorenyl, 2-fluorenyl, 3-fluorenyl, 4-fluorenyl, 9-fluorenyl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 1-anthryl, 2-anthryl, 9-anthryl, 1-phenanthryl, 2-phenanthryl, 3-phenanthryl, 4-phenanthryl, 9-phenanthryl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 3-fluoranthenyl, 4-fluoranthenyl, 8-fluoranthenyl, 9-fluoranthenyl, and benzofluoranthenyl. Herein, “(3- to 30-membered)heteroaryl(ene)” is meant to be an aryl group having a ring skeleton of 3 to 30 atoms and including at least one heteroatom selected from the group consisting of B, N, O, S, Si, P, Se, and Ge, preferably having a ring skeleton of 5 to 25 atoms. The number of heteroatoms is preferably from 1 to 4, and the heteroaryl(ene) may be a monocyclic ring system or a fused ring system condensed with one or more benzene rings, and may be partially saturated. Also, the above heteroaryl herein may be one formed by linking at least one heteroaryl group or aryl group to a heteroaryl group via a single bond(s). Examples of the heteroaryl specifically may be a monocyclic ring system heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyridinyl, pyrimidinyl, pyridazinyl, etc., and a fused ring system heteroaryl including benzofuranyl, benzothiophenyl, isobenzofuranyl, dibenzofuranyl, dibenzothiophenyl, benzoimidazolyl, benzothiazolyl, benzoisothiazolyl, benzoisoxazolyl, benzoxazolyl, imidazopyridinyl, isoindolyl, indolyl, benzoindolyl, indazolyl, benzothiadiazolyl, quinolyl, isoquinolyl, cinnolinyl, quinazolinyl, quinoxalinyl, carbazolyl, azacarbazolyl, benzocarbazolyl, dibenzocarbazolyl, phenoxazinyl, phenanthridinyl, benzodioxolyl, indolizidinyl, acridinyl, silafluorenyl, germafluorenyl, etc. More specifically, the heteroaryl may be 1-pyrrolyl, 2-pyrrolyl, 3-pyrrolyl, 2-pyridinyl, 3-pyridinyl, 4-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolizidinyl, 2-indolizidinyl, 3-indolizidinyl, 5-indolizidinyl, 6-indolizidinyl, 7-indolizidinyl, 8-indolizidinyl, 2-imidazopyridinyl, 3-imidazopyridinyl, 5-imidazopyridinyl, 6-imidazopyridinyl, 7-imidazopyridinyl, 8-imidazopyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazolyl-1-yl, azacarbazolyl-2-yl, azacarbazolyl-3-yl, azacarbazolyl-4-yl, azacarbazolyl-5-yl, azacarbazolyl-6-yl, azacarbazolyl-7-yl, azacarbazolyl-8-yl, azacarbazolyl-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-t-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-t-butyl-1-indolyl, 4-t-butyl-1-indolyl, 2-t-butyl-3-indolyl, 4-t-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. Additionally, “heteroaryl(ene)” can be classified into heteroaryl(ene) with electronic properties and heteroaryl(ene) with hole properties. A heteroaryl(ene) with electronic properties is an electron-rich substituent relative to the parent nucleus, and for example, it may be a substituted or unsubstituted pyridinyl, a substituted or unsubstituted pyrimidinyl, a substituted or unsubstituted triazinyl, a substituted or unsubstituted quinazolinyl, a substituted or unsubstituted quinoxalinyl, a substituted or unsubstituted quinolyl, etc. Heteroaryl(ene), which has hole characteristics, is an electron-deficient substituent relative to the parent nucleus, and for example, it may be a substituted or unsubstituted carbazolyl, a substituted or unsubstituted dibenzofuranyl, or a substituted or unsubstituted dibenzothiophenyl. Herein, the term “a fused ring of (C₃-C₃₀) aliphatic ring and (C₆-C₃₀) aromatic ring” means a functional group of a ring formed by fusing at least one aliphatic ring having 3 to 30, preferably 3 to 25, and more preferably 3 to 18 ring skeleton carbon atoms, and at least one aromatic ring having 6 to 30, preferably 6 to 25, and more preferably 6 to 18 ring skeleton carbon atoms. For example, the fused ring may be a fused ring of at least one benzene and at least one cyclohexane, or a fused ring of at least one naphthalene and at least one cyclopentane, etc. Herein, the carbon atoms in the fused ring of (C₃-C₃₀) aliphatic ring and (C₆-C₃₀) aromatic ring may be replaced with at least one heteroatom selected from B, N, O, S, Si, and P, preferably at least one heteroatom selected from N, O, and S. The term “halogen” in the present disclosure includes F, Cl, Br, and I.

In addition, “ortho-” (“o-”), “meta-” (“m-”), and “para-” (“p-”) are meant to signify the substitution position of all substituents. The “ortho-” configuration indicates a compound with substituents which are adjacent to each other, e.g., at the 1 and 2 positions on benzene. The “meta-” configuration indicates the next substitution position of the immediately adjacent substitution position, e.g., a compound with substituents at the 1 and 3 positions on benzene. The “para-” configuration indicates the next substitution position from the “meta-” position, e.g., a compound with substituents at the 1 and 4 positions on benzene.

Herein, the term “a ring formed in linking to an adjacent substituent” means a substituted or unsubstituted (3- to 30-membered) mono- or polycyclic, alicyclic, aromatic ring, or a combination thereof, formed by linking or fusing two or more adjacent substituents, and preferably may be a substituted or unsubstituted (3- to 26-membered) mono- or polycyclic, alicyclic, aromatic ring, or a combination thereof. Further, the formed ring may include at least one heteroatom selected from the group consisting of B, N, O, S, Si, and P, preferably, N, O, and S. According to one embodiment of the present disclosure, the number of atoms in the ring skeleton is 5 to 20, according to another embodiment of the present disclosure, the number of atoms in the ring skeleton is 5 to 15.

In addition, “substituted” in the expression “substituted or unsubstituted” means that a hydrogen atom in a certain functional group is replaced with another atom or functional group, i.e., a substituent. Unless otherwise specified, the substituent may replace hydrogen at a position where the substituent may be substituted without limitation, and when two or more hydrogen atoms in a functional group are each replaced with a substituent, each substituent may be the same or different. The maximum number of substituents that can be substituted for a certain functional group may be the total number of valences that can be substituted for each atom forming the functional group. In the formulas of the present disclosure, the substituted arene, the substituted heteroarene, the substituted alkyl, the substituted aryl(ene), the substituted heteroaryl(ene), the substituted cycloalkyl, the substituted alkoxy, the substituted trialkylsilyl, the substituted dialkylarylsilyl, the substituted alkyldiarylsilyl, the substituted triarylsilyl, the substituted fused ring of aliphatic ring and aromatic ring, the substituted mono- or dialkylamino, the substituted mono- or diarylamino, the substituted alkylarylamino, the substituted mono- or diheteroarylamino, and the substituted arylheteroarylamino each independently is preferably substituted with at least one selected from the group consisting of deuterium; halogen; cyano; carboxyl; nitro; hydroxy; (C₁-C₃₀)alkyl; halo(C₁-C₃₀)alkyl; (C₂-C₃₀)alkenyl; (C₂-C₃₀)alkynyl; (C₁-C₃₀)alkoxy; (C₁-C₃₀)alkylthio; (C₃-C₃₀)cycloalkyl; (C₃-C₃₀)cycloalkenyl; (3- to 7-membered)heterocycloalkyl; (C₆-C₃₀)aryloxy; (C₆-C₃₀)arylthio; (5- to 30-membered)heteroaryl substituted or unsubstituted with (C₆-C₃₀)aryl; (C₆-C₃₀)aryl substituted or unsubstituted with (5- to 30-membered)heteroaryl; tri(C₁-C₃₀)alkylsilyl; tri(C₆-C₃₀)arylsilyl; di(C₁-C₃₀)alkyl(C₆-C₃₀)arylsilyl; (C₁-C₃₀)alkyldi(C₆-C₃₀)arylsilyl; a fused ring of (C₃-C₃₀)aliphatic ring and (C₆-C₃₀)aromatic ring; amino; mono- or di(C₁-C₃₀)alkylamino; a substituted or unsubstituted mono- or di(C₆-C₃₀)arylamino; (C₁-C₃₀)alkyl(C₆-C₃₀)arylamino; mono- or di(3- to 30-membered)heteroarylamino; (C₁-C₃₀)alkyl(3- to 30-membered)heteroarylamino; (C₆-C₃₀)aryl(3- to 30-membered)heteroarylamino; (C₁-C₃₀)alkylcarbonyl; (C₁-C₃₀)alkoxycarbonyl; (C₆-C₃₀)arylcarbonyl; (C₆-C₃₀)arylphosphinyl, di(C₆-C₃₀)arylboronyl; di(C₁-C₃₀)alkylboronyl; (C₁-C₃₀)alkyl(C₆-C₃₀)arylboronyl; (C₆-C₃₀)ar(C₁-C₃₀)alkyl; (C₁-C₃₀)alkyl(C₆-C₃₀)aryl, and a combination thereof. For example, the substituent may be a substituted or unsubstituted methyl; a substituted or unsubstituted phenyl; a substituted or unsubstituted naphthyl; a substituted or unsubstituted biphenyl; a substituted or unsubstituted diphenylfluorenyl; a substituted or unsubstituted dimethylfluorenyl; a substituted or unsubstituted pyridinyl; a substituted or unsubstituted dibenzofuranyl; a substituted or unsubstituted dibenzothiophenyl; or a substituted or unsubstituted carbazolyl; etc.

Herein, when no substituent is indicated in the formula or compound structure, it may mean that all positions that can be substituted are hydrogen or deuterium. That is, some hydrogen atoms may be deuterium, which is an isotope of hydrogen, in which the deuterium content may be 0% to 100%. Herein, when no substituent is indicated in the formula or compound structure, hydrogen and deuterium may coexist in the compound unless deuterium is explicitly excluded, such as when the deuterium content is 0%, the hydrogen content is 100%, or all substituents are specified as hydrogen. The deuterium, which may be represented as hydrogen-2, and the element symbol of which may also be written as D or ²H, is one of the isotopes of hydrogen and has a deuteron, consisting of one proton and one neutron, as its nucleus. The isotopes, which means elements with the same atomic number (Z) but different mass numbers (A), may also be interpreted as elements with the same number of protons but different numbers of neutrons.

As used herein, “a combination thereof” refers to a combination of one or more elements from the corresponding list to form a known or chemically stable arrangement that may be envisioned by a person skilled in the art from the corresponding list. For example, alkyl and deuterium may be combined to form an alkyl group that is partially or fully deuterated; halogen and alkyl may be combined to form a halogenated alkyl substituent; and halogen, alkyl, and aryl may be combined to form halogenated arylalkyl. For example, preferred combinations of substituents include up to 50 atoms other than hydrogen or deuterium, or up to 40 atoms other than hydrogen or deuterium, or up to 30 atoms other than hydrogen or deuterium. Alternatively, in many cases, the preferred combination of substituents may be those containing up to 20 atoms other than hydrogen or deuterium.

In the formulas of the present disclosure, when a plurality of substituents represented by the same symbol are present, each of the substituents represented by the same symbol may be the same or different.

Hereinafter, an organic electroluminescent compound according to one embodiment will be described.

The compound according to one embodiment is represented by the following Formula 1.

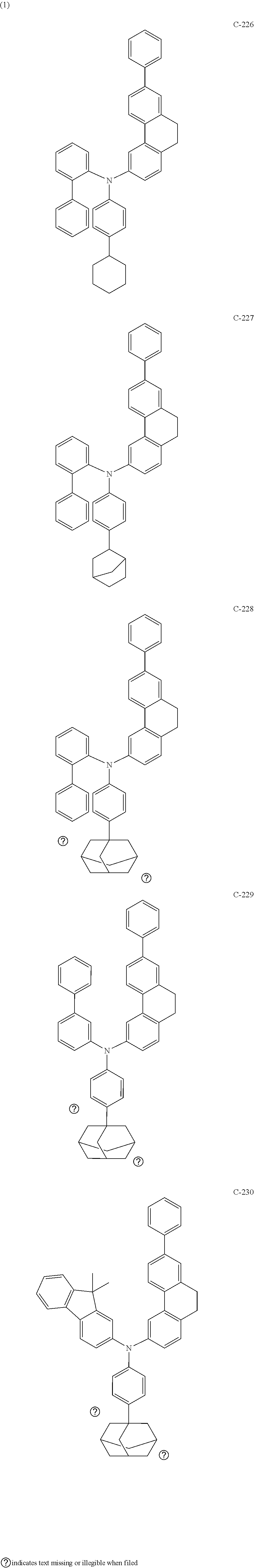 [SMILES: c1ccc(-c2ccc3c(c2)CCc2ccc(N(c4ccc(C5CCCCC5)cc4)c4ccccc4-c4ccccc4)cc2-3)cc1 ; c1ccc(-c2ccc3c(c2)CCc2ccc(N(c4ccc(C5CC6CCC5C6)cc4)c4ccccc4-c4ccccc4)cc2-3)cc1 ; c1ccc(-c2ccc3c(c2)CCc2ccc(N(c4ccc(C56CC7CC(CC(C7)C5)C6)cc4)c4ccccc4-c4ccccc4)cc2-3)cc1 ; c1ccc(-c2cccc(N(c3ccc(C45CC6CC(CC(C6)C4)C5)cc3)c3ccc4c(c3)-c3ccc(-c5ccccc5)cc3CC4)c2)cc1 ; CC1(C)c2ccccc2-c2ccc(N(c3ccc(C45CC6CC(CC(C6)C4)C5)cc3)c3ccc4c(c3)-c3ccc(-c5ccccc5)cc3CC4)cc21]

In Formula 1,
ring A and ring B each independently represent a substituted or unsubstituted (C₆-C₃₀)arene, or a substituted or unsubstituted (3- to 30-membered)heteroarene;
R₁ to R₄, Rₐ, and R_{b} each independently represent hydrogen, deuterium, halogen, cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring of (C₃-C₃₀) aliphatic ring and (C₆-C₃₀) aromatic ring, a substituted or unsubstituted mono- or di(C₁-C₃₀)alkylamino, a substituted or unsubstituted mono- or di(C₆-C₃₀)arylamino, a substituted or unsubstituted (C₁-C₃₀)alkyl(C₆-C₃₀)arylamino, a substituted or unsubstituted mono- or di(3-to 30-membered)heteroarylamino, or a substituted or unsubstituted (C₆-C₃₀)aryl(3- to 30-membered)heteroarylamino; or may be linked to the adjacent substituents to form a ring(s);
provided that at least one of R₁ to R₄, Rₐ, and R_{b} is represented by the following Formula 1-1;

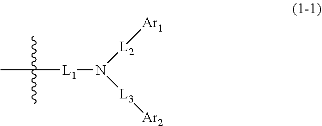

In Formula 1-1,
L₁ to L₃ each independently represent a single bond, a substituted or unsubstituted (C₆-C₃₀)arylene, or a substituted or unsubstituted (3- to 30-membered)heteroarylene;
Ar₁ and Ar₂ each independently represent a substituted or unsubstituted (C₆-C₃₀)aryl, a substituted or unsubstituted (3- to 30-membered)heteroaryl, or a substituted or unsubstituted (C₃-C₃₀)cycloalkyl;
provided that at least one of Ar₁ and Ar₂ is a substituted or unsubstituted (C₃-C₃₀)cycloalkyl; and
n and m are integers of 1 to 8, and when n and m are integers greater than or equal to 2, each of Rₐ and each of R_{b} may be the same or different.

In one embodiment, ring A and ring B each independently may be a substituted or unsubstituted (C₆-C₃₀)arene, or a substituted or unsubstituted (3- to 30-membered)heteroarene. For example, ring A and ring B each independently may be benzene ring, naphthalene ring, phenanthrene ring, dibenzothiophene ring, dibenzofuran ring, carbazole ring, or fluorene ring.

In one embodiment, R₁ to R₄ each independently may be hydrogen, deuterium, a substituted or unsubstituted (C₁-C₃₀)alkyl, a substituted or unsubstituted (C₆-C₃₀)aryl, a substituted or unsubstituted (3- to 30-membered)heteroaryl, or a substituted or unsubstituted (C₃-C₃₀)cycloalkyl, preferably hydrogen, deuterium, or a substituted or unsubstituted (C₁-C₁₀)alkyl, more preferably hydrogen, or a substituted or unsubstituted (C₁-C₄)alkyl. For example, R₁ to R₄ each independently may be hydrogen, or a substituted or unsubstituted methyl.

In one embodiment, Rₐ, and R_{b} each independently may be hydrogen, deuterium, a substituted or unsubstituted (C₆-C₃₀)aryl, a substituted or unsubstituted (C₅-C₃₀)cycloalkyl, a substituted or unsubstituted mono- or di(C₁-C₃₀)alkylamino, a substituted or unsubstituted mono- or di(C₆-C₃₀)arylamino, a substituted or unsubstituted (C₁-C₃₀)alkyl(C₆-C₃₀)arylamino, a substituted or unsubstituted mono- or di(5- to 30-membered)heteroarylamino, a substituted or unsubstituted (C₆-C₃₀)aryl(5- to 30-membered)heteroarylamino, or may be linked to the adjacent substituents to form a substituted or unsubstituted (5- to 30-membered) monocyclic or polycyclic aromatic ring(s), preferably hydrogen, deuterium, a substituted or unsubstituted (C₆-C₂₅)aryl, a substituted or unsubstituted (C₅-C₃₀)cycloalkyl, a substituted or unsubstituted mono- or di(C₆-C₃₀)arylamino, a substituted or unsubstituted mono- or di(5- to 30-membered)heteroarylamino, a substituted or unsubstituted (C₆-C₃₀)aryl(5- to 30-membered)heteroarylamino, or may be linked to the adjacent substituents to form a substituted or unsubstituted (5- to 25-membered) monocyclic or polycyclic aromatic ring(s), more preferably hydrogen, a substituted or unsubstituted (C₆-C₁₈)aryl, a substituted or unsubstituted (C₅-C₃₀)cycloalkyl, a substituted or unsubstituted di(C₆-C₃₀)arylamino, a substituted or unsubstituted di(5- to 30-membered)heteroarylamino, a substituted or unsubstituted (C₆-C₃₀)aryl(5- to 30-membered)heteroarylamino, or may be linked to the adjacent substituents to form a substituted or unsubstituted (5- to 18-membered) monocyclic or polycyclic aromatic ring(s). For example, Rₐ and R_{b} each independently may be hydrogen or a substituted or unsubstituted phenyl, a substituted or unsubstituted p-biphenyl, a substituted or unsubstituted m-biphenyl, a substituted or unsubstituted o-biphenyl, a substituted or unsubstituted naphthyl, a substituted or unsubstituted adamantanyl, a substituted or unsubstituted cyclohexyl, a substituted or unsubstituted dibenzofuranyl, a substituted or unsubstituted phenanthrenyl; or a substituted or unsubstituted benzene ring, a substituted or unsubstituted naphthalene ring, a substituted or unsubstituted 1,2,3,4-tetrahydronaphthalene ring, a substituted or unsubstituted benzofuran ring, a substituted or unsubstituted benzothiophene ring, a substituted or unsubstituted indole ring, or a substituted or unsubstituted indene ring linked to the adjacent substituents.

In one embodiment, at least one of Rₐ, and R_{b} may be represented by the Formula 1-1.

In one embodiment, L₁, L₂, and L₃ each independently may be a single bond, a substituted or unsubstituted (C₆-C₃₀)arylene, or a substituted or unsubstituted (5- to 30-membered)heteroarylene, preferably a single bond, a substituted or unsubstituted (C₆-C₂₅)arylene, or a substituted or unsubstituted (5- to 25-membered)heteroarylene, more preferably a single bond, a substituted or unsubstituted (C₆-C₁₈)arylene, or a substituted or unsubstituted (5- to 18-membered)heteroarylene. For example, L₁ may be a substituted or unsubstituted phenylene, a substituted or unsubstituted p-biphenylene, a substituted or unsubstituted m-biphenylene, a substituted or unsubstituted o-biphenylene, a substituted or unsubstituted naphthylene, a substituted or unsubstituted dibenzofuranylene, a substituted or unsubstituted dibenzothiophenylene, a substituted or unsubstituted dimethylfluorenylene, or a substituted or unsubstituted carbazolylene, and L₂ and L₃ each independently may be a substituted or unsubstituted phenylene, or a substituted or unsubstituted biphenylene.

In one embodiment, Ar₁ and Ar₂ each independently may be a substituted or unsubstituted (C₆-C₃₀)aryl, a substituted or unsubstituted (5- to 30-membered)heteroaryl, or a substituted or unsubstituted (C₃-C₃₀)cycloalkyl, preferably a substituted or unsubstituted (C₆-C₂₅)aryl, a substituted or unsubstituted (5- to 25-membered)heteroaryl, or a substituted or unsubstituted (C₅-C₃₀)cycloalkyl, more preferably a substituted or unsubstituted (C₆-C₂₅)aryl, a substituted or unsubstituted (5- to 18-membered)heteroaryl, or a substituted or unsubstituted (C₅-C₃₀)cycloalkyl. For example, Ar₁ and Ar₂ each independently may be a substituted or unsubstituted phenyl, a substituted or unsubstituted p-biphenyl a substituted or unsubstituted m-biphenyl, a substituted or unsubstituted o-biphenyl, a substituted or unsubstituted m-terphenyl, a substituted or unsubstituted o-terphenyl, a substituted or unsubstituted phenanthrenyl, a substituted or unsubstituted dimethylfluorenyl, a substituted or unsubstituted diethylfluorenyl, a substituted or unsubstituted spirobifluorenyl, a substituted or unsubstituted cyclopropyl, a substituted or unsubstituted cyclobutyl, a substituted or unsubstituted cyclopentyl, a substituted or unsubstituted cyclohexyl, a substituted or unsubstituted cycloheptyl, a substituted or unsubstituted cyclooctyl, a substituted or unsubstituted bicyclo[1.1.1]pentyl, a substituted or unsubstituted bicyclo[2.1.1]hexyl, a substituted or unsubstituted bicyclo[2.2.1]heptyl, a substituted or unsubstituted bicyclo[3.1.1]heptyl, a substituted or unsubstituted bicyclo[2.2.2]octyl, a substituted or unsubstituted bicyclo[3.2.1]octyl, a substituted or unsubstituted adamantanyl, a substituted or unsubstituted 2,3-dihydro-1H-indenyl, a substituted or unsubstituted 1,2,3,4-tetrahydronaphthalenyl, a substituted or unsubstituted 6,7,8,9-tetrahydro-5H-benzo[7]annulenyl, a substituted or unsubstituted dibenzofuranyl, a substituted or unsubstituted dibenzothiophenyl, or a substituted or unsubstituted carbazolyl.

In one embodiment, both Ar₁ and Ar₂ may be a substituted or unsubstituted (C₃-C₃₀)cycloalkyl.

In one embodiment, at least one of Ar₁ and Ar₂ may be cyclopropyl, cyclobutyl, cyclopentyl, cyclohexyl, cycloheptyl, cyclooctyl, bicyclo[2.1.1]hexyl, bicyclo[2.2.1]heptyl, bicyclo[3.1.1]heptyl, bicyclo[2.2.2]octyl, bicyclo[3.2.1]octyl, adamantanyl, 2,3-dihydro-1H-indenyl, 1,2,3,4-tetrahydronaphthalenyl, or 6,7,8,9-tetrahydro-5H-benzo[7]annulenyl.

In one embodiment, an organic electroluminescent compound according to Formula 1 may be substituted with at least one deuterium.

According to one embodiment, the organic electroluminescent compound represented by Formula 1 may have a refractive index of 1.71 or less. When the refractive index of the organic electroluminescent compound is 1.71 or less, light reflection loss can be reduced, allowing more light to be extracted externally, and this enhanced light extraction effect can improve the current efficiency of the organic electroluminescent device.

The organic electroluminescent compound represented by Formula 1 according to one embodiment may be represented by any one of the following Formulas 2-1 to 2-11.

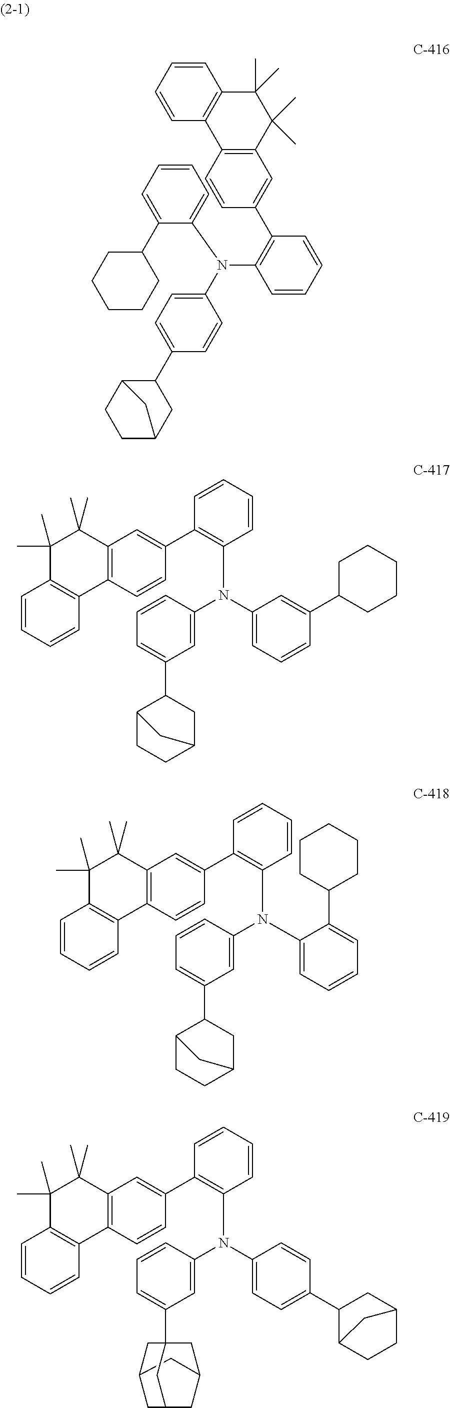

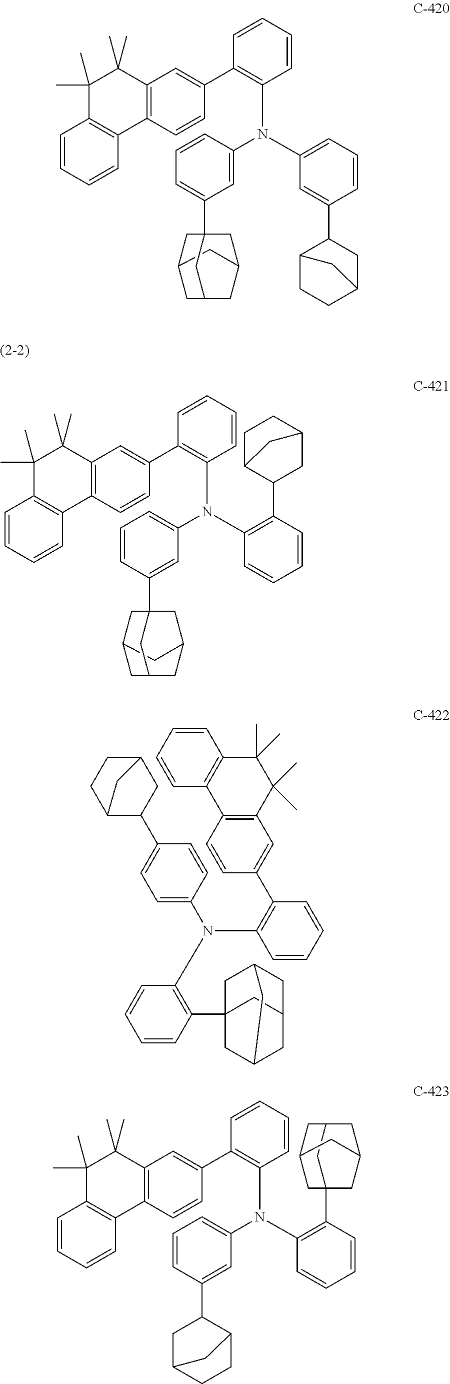

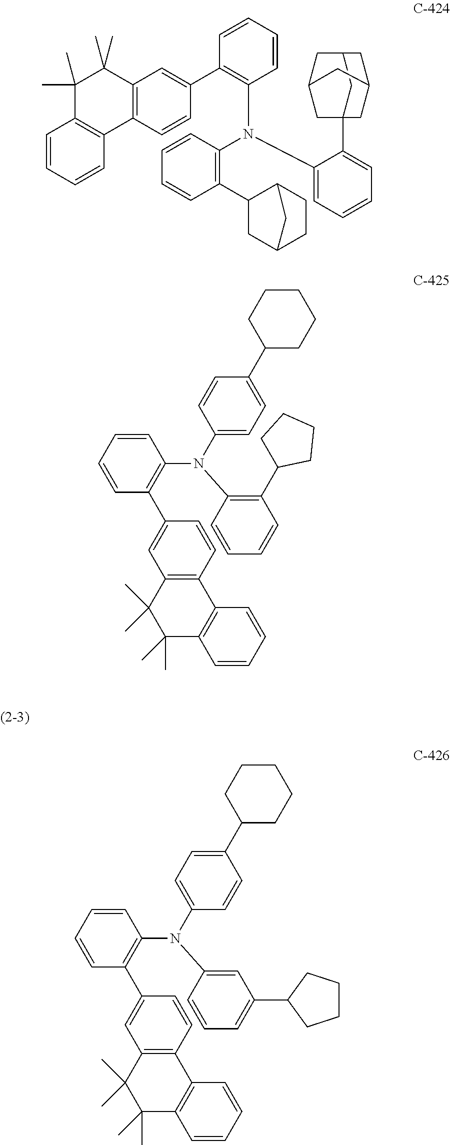

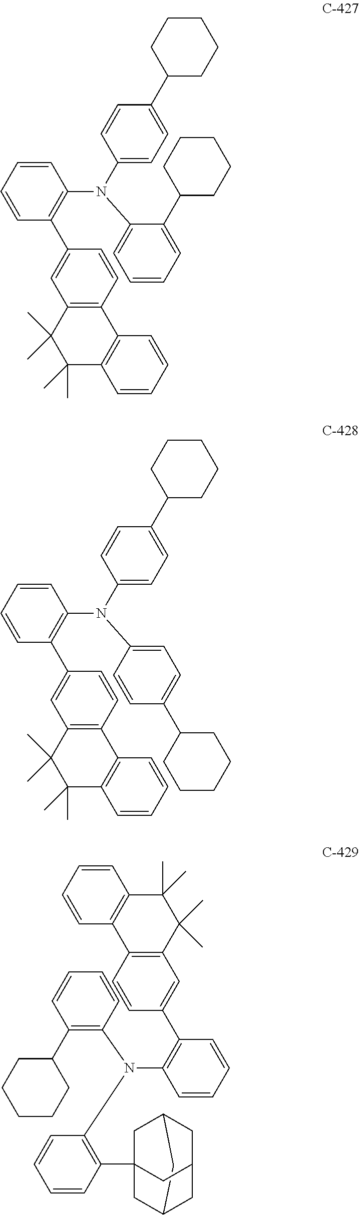 [SMILES: CC1(C)c2ccccc2-c2ccc(-c3ccccc3N(c3ccc(C4CCCCC4)cc3)c3ccccc3C3CCCCC3)cc2C1(C)C ; CC1(C)c2ccccc2-c2ccc(-c3ccccc3N(c3ccc(C4CCCCC4)cc3)c3ccc(C4CCCCC4)cc3)cc2C1(C)C ; CC1(C)c2ccccc2-c2ccc(-c3ccccc3N(c3ccccc3C3CCCCC3)c3ccccc3C34CC5CC(CC(C5)C3)C4)cc2C1(C)C]

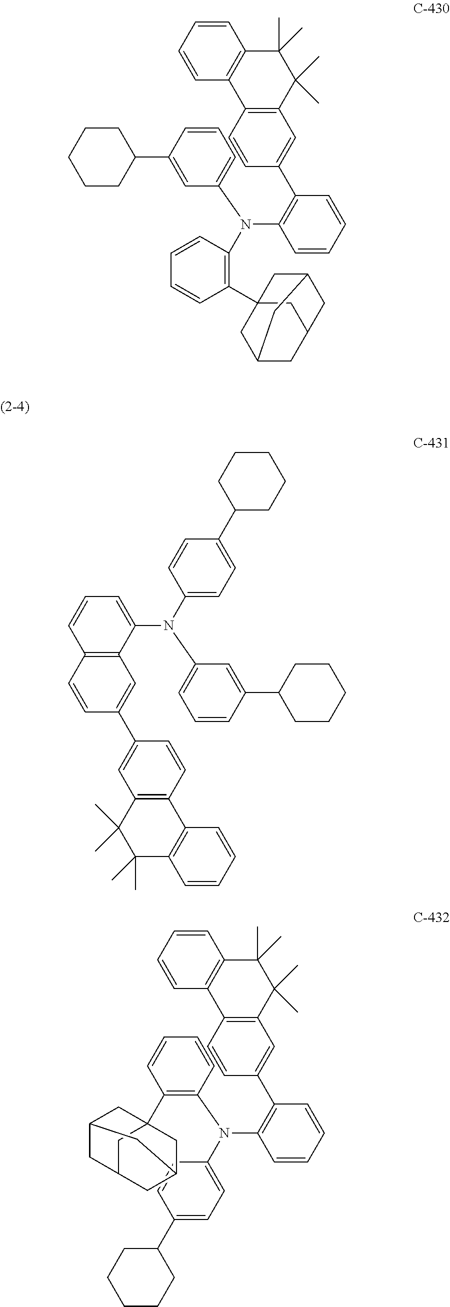 [SMILES: CC1(C)c2ccccc2-c2ccc(-c3ccccc3N(c3cccc(C4CCCCC4)c3)c3ccccc3C34CC5CC(CC(C5)C3)C4)cc2C1(C)C ; CC1(C)c2ccccc2-c2ccc(-c3ccc4cccc(N(c5ccc(C6CCCCC6)cc5)c5cccc(C6CCCCC6)c5)c4c3)cc2C1(C)C ; CC1(C)c2ccccc2-c2ccc(-c3ccccc3N(c3ccc(C4CCCCC4)cc3)c3ccccc3C34CC5CC(CC(C5)C3)C4)cc2C1(C)C]

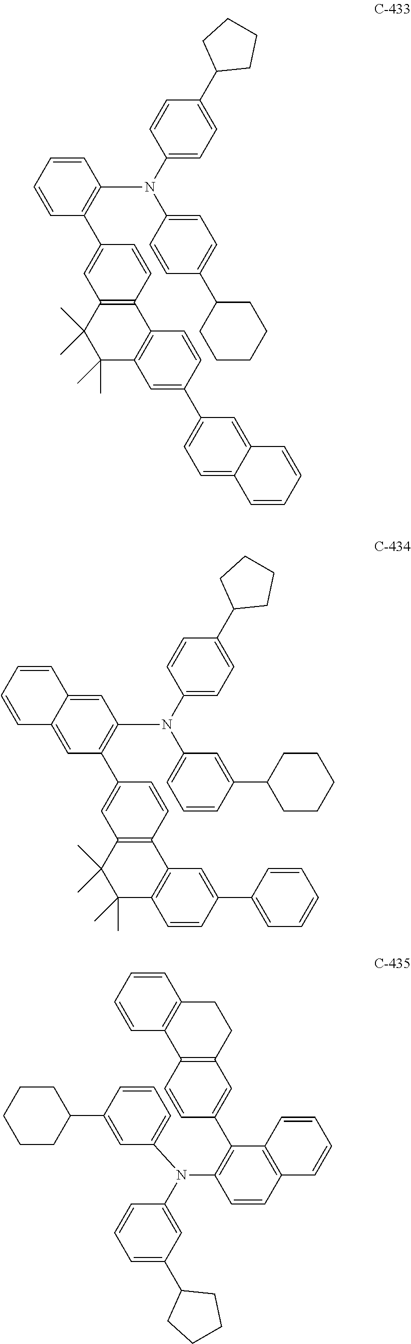

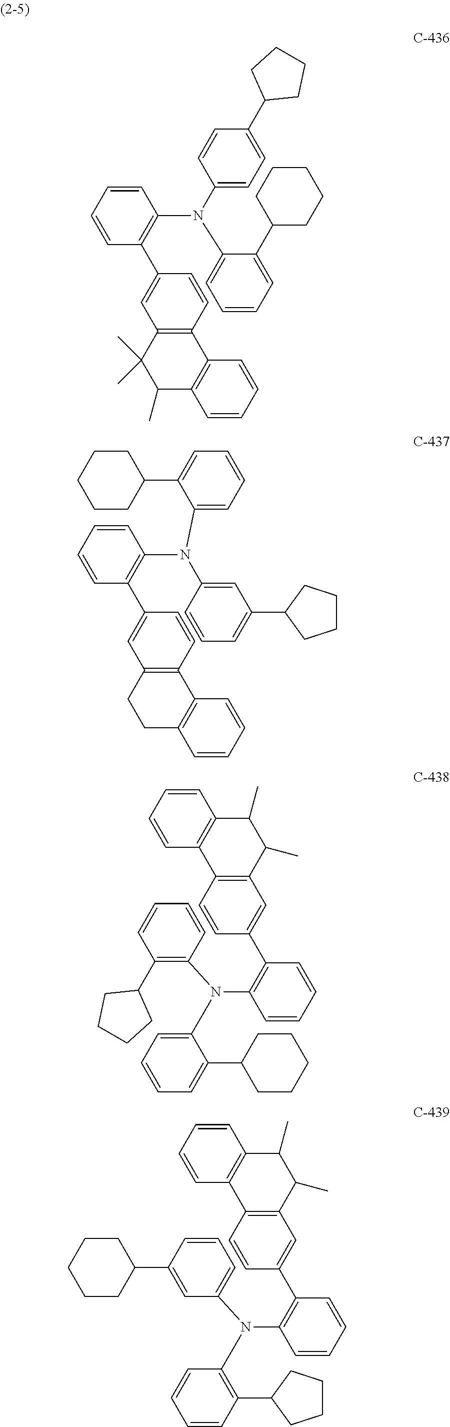

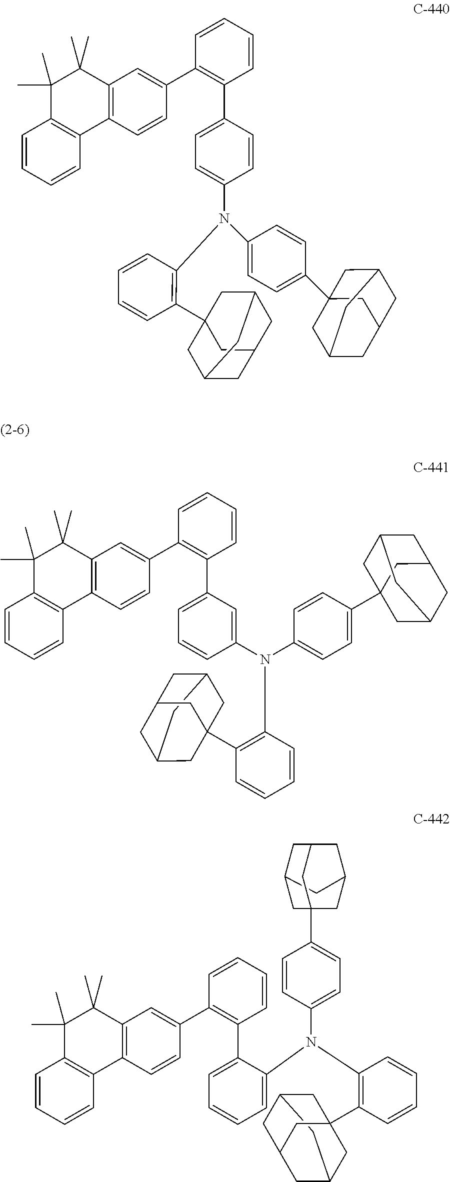 [SMILES: CC1(C)c2ccccc2-c2ccc(-c3ccccc3-c3ccc(N(c4ccc(C56CC7CC(CC(C7)C5)C6)cc4)c4ccccc4C45CC6CC(CC(C6)C4)C5)cc3)cc2C1(C)C ; CC1(C)c2ccccc2-c2ccc(-c3ccccc3-c3cccc(N(c4ccc(C56CC7CC(CC(C7)C5)C6)cc4)c4ccccc4C45CC6CC(CC(C6)C4)C5)c3)cc2C1(C)C ; CC1(C)c2ccccc2-c2ccc(-c3ccccc3-c3ccccc3N(c3ccc(C45CC6CC(CC(C6)C4)C5)cc3)c3ccccc3C34CC5CC(CC(C5)C3)C4)cc2C1(C)C]

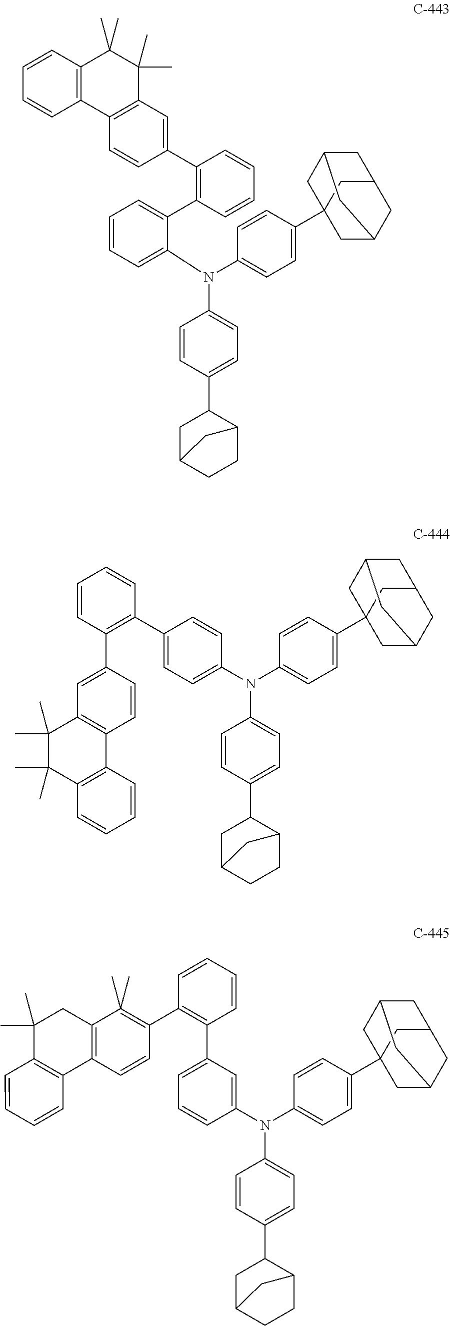

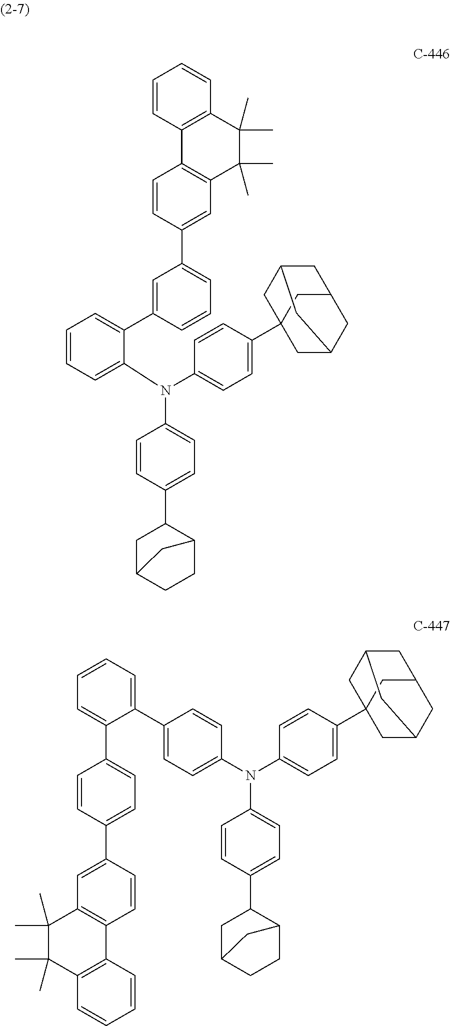 [SMILES: CC1(C)c2ccccc2-c2ccc(-c3cccc(-c4ccccc4N(c4ccc(C5CC6CCC5C6)cc4)c4ccc(C56CC7CC(CC(C7)C5)C6)cc4)c3)cc2C1(C)C ; CC1(C)c2ccccc2-c2ccc(-c3ccc(-c4ccccc4-c4ccc(N(c5ccc(C6CC7CCC6C7)cc5)c5ccc(C67CC8CC(CC(C8)C6)C7)cc5)cc4)cc3)cc2C1(C)C]

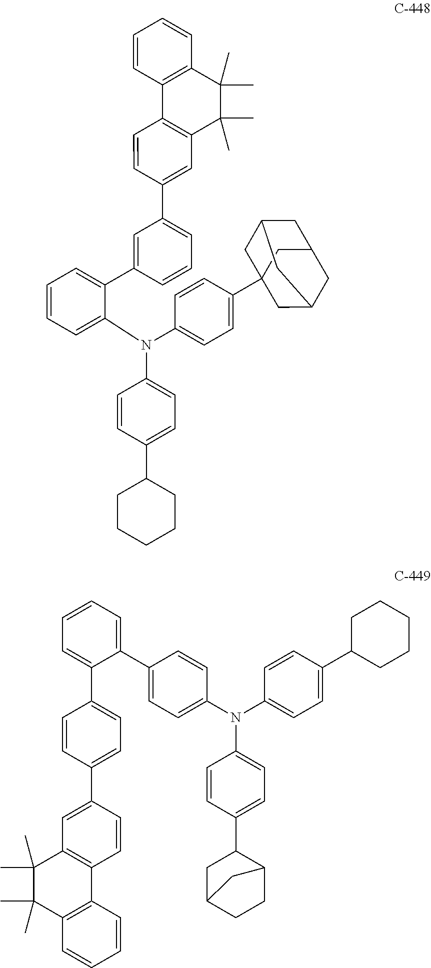 [SMILES: CC1(C)c2ccccc2-c2ccc(-c3cccc(-c4ccccc4N(c4ccc(C5CCCCC5)cc4)c4ccc(C56CC7CC(CC(C7)C5)C6)cc4)c3)cc2C1(C)C ; CC1(C)c2ccccc2-c2ccc(-c3ccc(-c4ccccc4-c4ccc(N(c5ccc(C6CCCCC6)cc5)c5ccc(C6CC7CCC6C7)cc5)cc4)cc3)cc2C1(C)C]

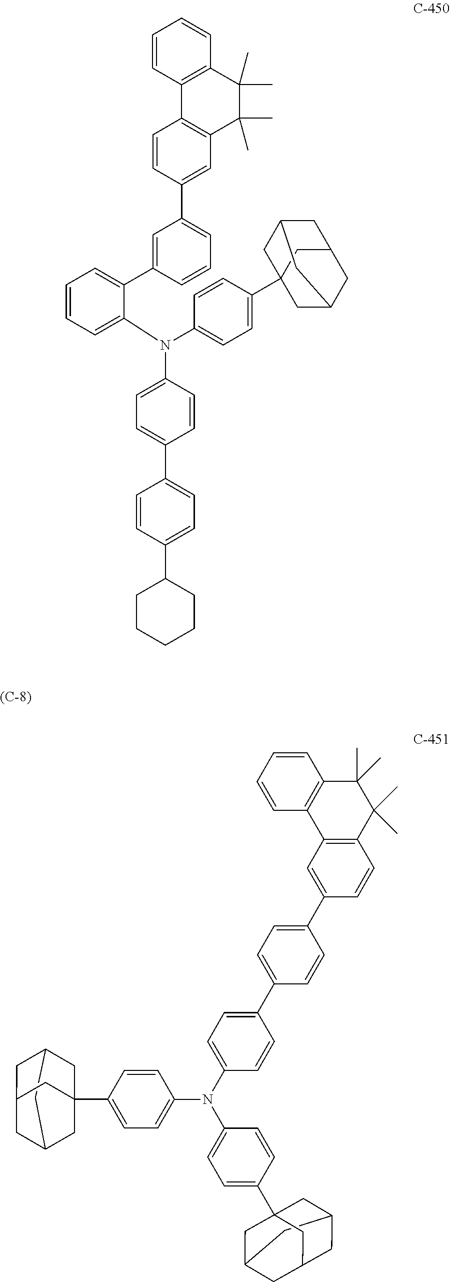 [SMILES: CC1(C)c2ccccc2-c2ccc(-c3cccc(-c4ccccc4N(c4ccc(-c5ccc(C6CCCCC6)cc5)cc4)c4ccc(C56CC7CC(CC(C7)C5)C6)cc4)c3)cc2C1(C)C ; "(C-8)" ; CC1(C)c2ccccc2-c2cc(-c3ccc(-c4ccc(N(c5ccc(C67CC8CC(CC(C8)C6)C7)cc5)c5ccc(C67CC8CC(CC(C8)C6)C7)cc5)cc4)cc3)ccc2C1(C)C]

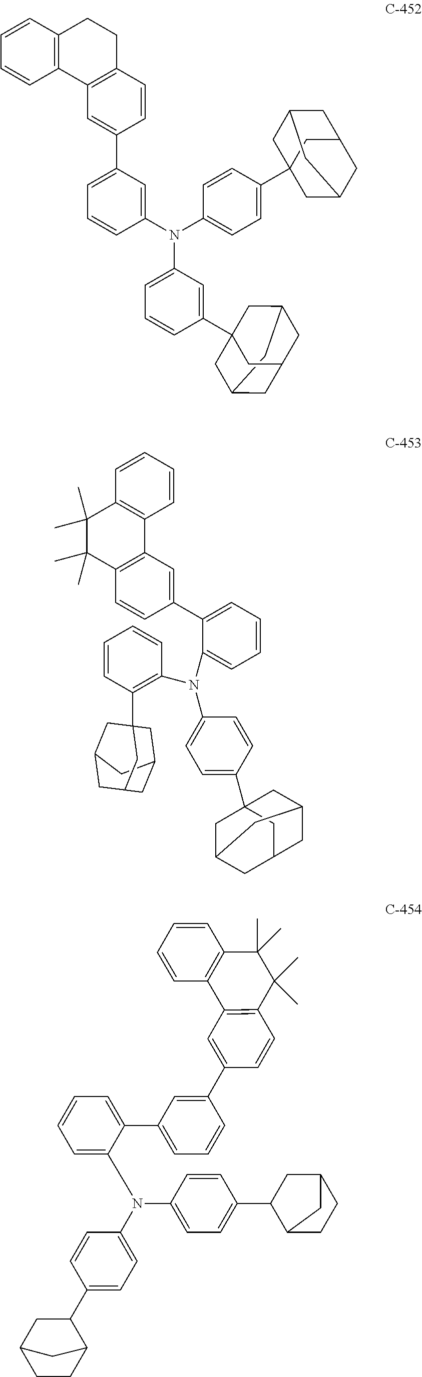 [SMILES: c1cc(-c2ccc3c(c2)-c2ccccc2CC3)cc(N(c2ccc(C34CC5CC(CC(C5)C3)C4)cc2)c2cccc(C34CC5CC(CC(C5)C3)C4)c2)c1 ; CC1(C)c2ccccc2-c2cc(-c3ccccc3N(c3ccc(C45CC6CC(CC(C6)C4)C5)cc3)c3ccccc3C34CC5CC(CC(C5)C3)C4)ccc2C1(C)C ; CC1(C)c2ccccc2-c2cc(-c3cccc(-c4ccccc4N(c4ccc(C5CC6CCC5C6)cc4)c4ccc(C5CC6CCC5C6)cc4)c3)ccc2C1(C)C]

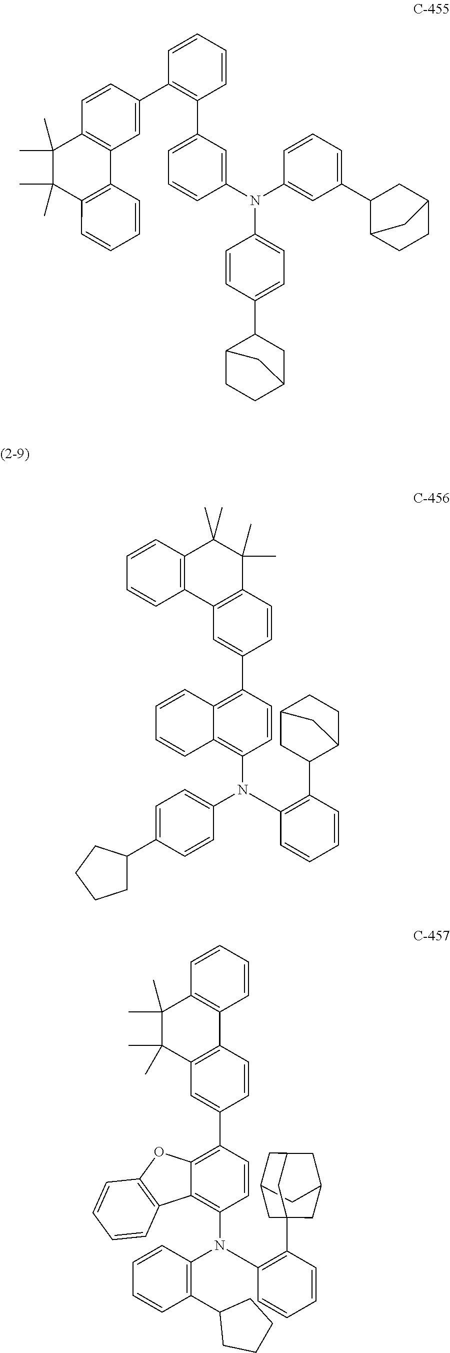 [SMILES: CC1(C)c2ccccc2-c2cc(-c3ccccc3-c3cccc(N(c4ccc(C5CC6CCC5C6)cc4)c4cccc(C5CC6CCC5C6)c4)c3)ccc2C1(C)C ; CC1(C)c2ccccc2-c2cc(-c3ccc(N(c4ccc(C5CCCC5)cc4)c4ccccc4C4CC5CCC4C5)c4ccccc34)ccc2C1(C)C ; CC1(C)c2ccccc2-c2ccc(-c3ccc(N(c4ccccc4C4CCCC4)c4ccccc4C45CC6CC(CC(C6)C4)C5)c4c3oc3ccccc34)cc2C1(C)C]

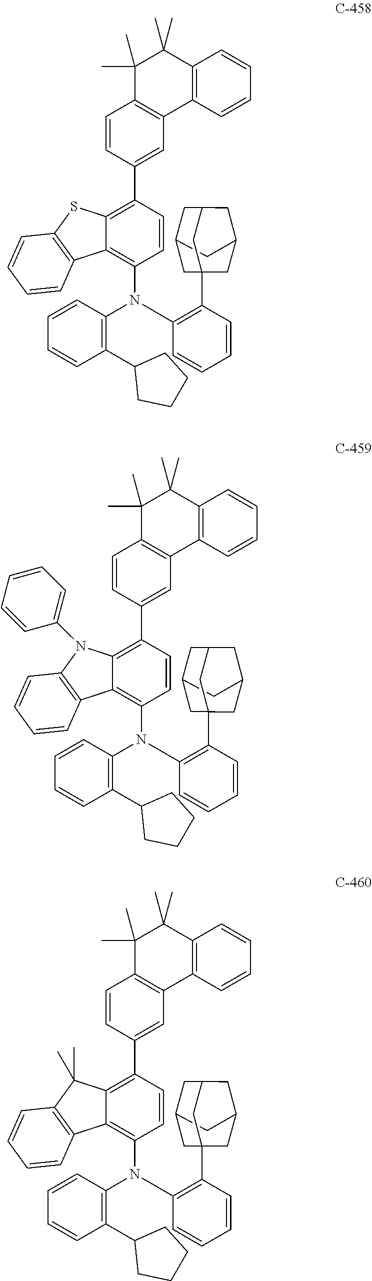

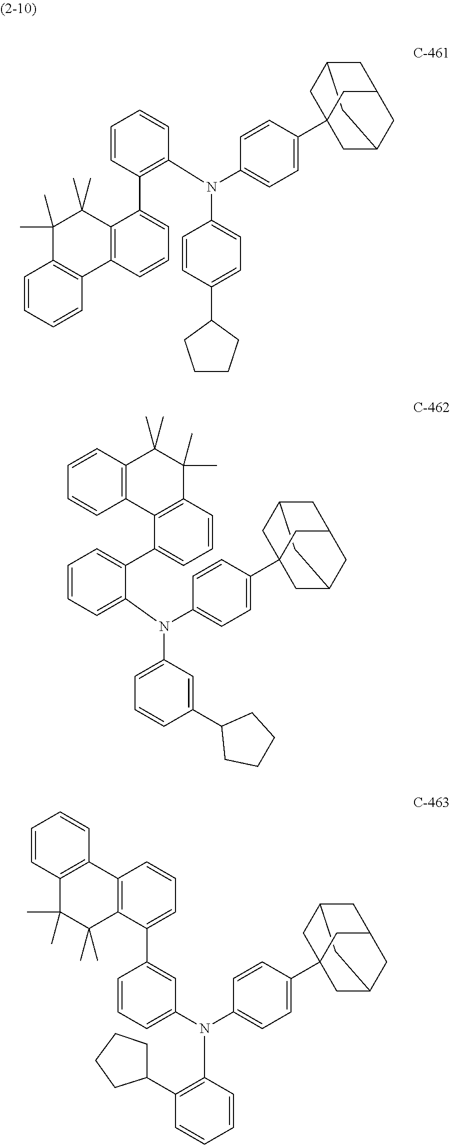 [SMILES: CC1(C)c2ccccc2-c2cccc(-c3ccccc3N(c3ccc(C4CCCC4)cc3)c3ccc(C45CC6CC(CC(C6)C4)C5)cc3)c2C1(C)C ; CC1(C)c2ccccc2-c2c(-c3ccccc3N(c3ccc(C45CC6CC(CC(C6)C4)C5)cc3)c3cccc(C4CCCC4)c3)cccc2C1(C)C ; CC1(C)c2ccccc2-c2cccc(-c3cccc(N(c4ccc(C56CC7CC(CC(C7)C5)C6)cc4)c4ccccc4C4CCCC4)c3)c2C1(C)C]

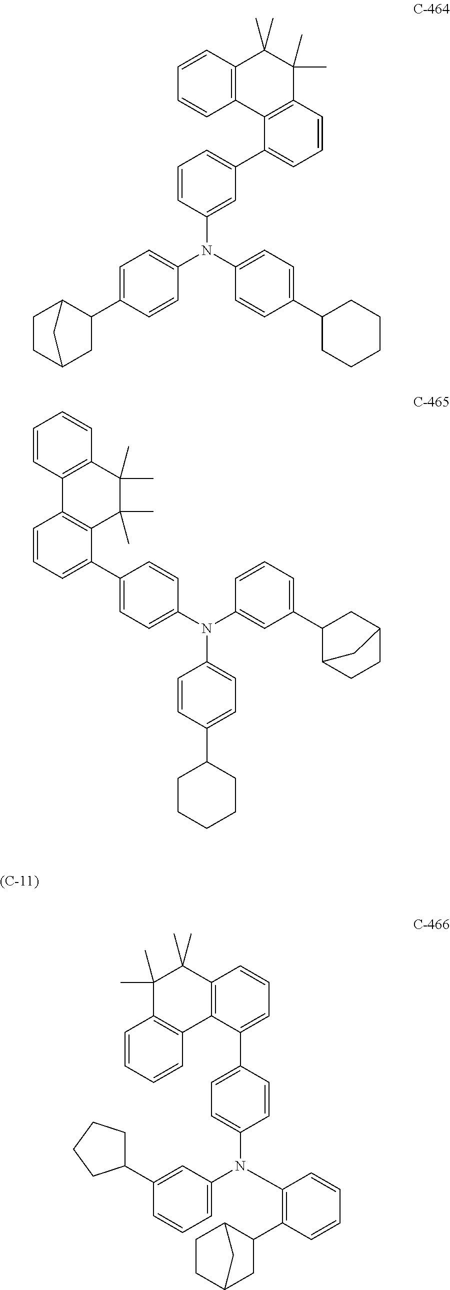

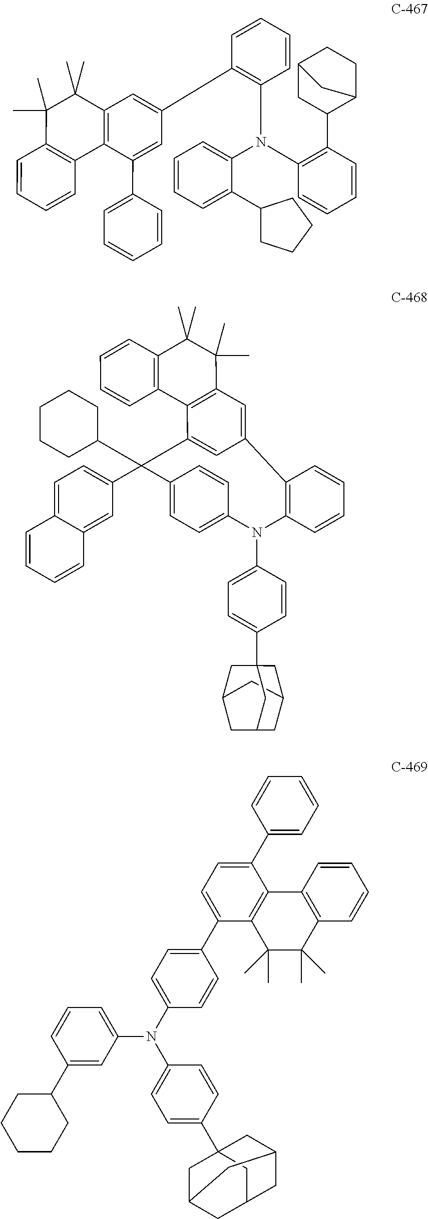 [SMILES: CC1(C)c2ccccc2-c2c(-c3ccccc3)cc(-c3ccccc3N(c3ccccc3C3CCCC3)c3ccccc3C3CC4CCC3C4)cc2C1(C)C ; CC1(C)c2ccccc2-c2c3cc(cc2C1(C)C)-c1ccccc1N(c1ccc(C24CC5CC(CC(C5)C2)C4)cc1)c1ccc(cc1)C3(c1ccc2ccccc2c1)C1CCCCC1 ; CC1(C)c2ccccc2-c2c(-c3ccccc3)ccc(-c3ccc(N(c4ccc(C56CC7CC(CC(C7)C5)C6)cc4)c4cccc(C5CCCCC5)c4)cc3)c2C1(C)C]

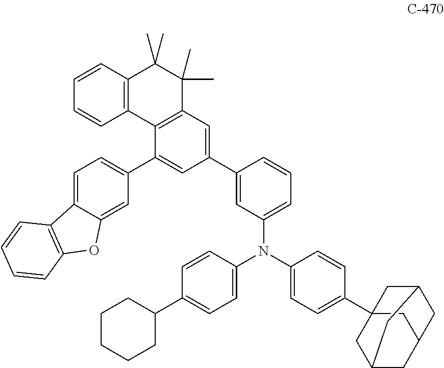 [SMILES: CC1(C)c2ccccc2-c2c(-c3ccc4c(c3)oc3ccccc34)cc(-c3cccc(N(c4ccc(C5CCCCC5)cc4)c4ccc(C56CC7CC(CC(C7)C5)C6)cc4)c3)cc2C1(C)C]

In Formulas 2-1 to 2-11,
R₁ to R₄ are as defined in Formula 1; and
R₅ to R₂₀ are as defined for Rₐ in Formula 1;
provided that at least one of R₁ to R₂₀ is represented by Formula 1-1.

According to one embodiment, the organic electroluminescent compound represented by Formula 1 may be represented by any one of the following Formulas 3-1 to

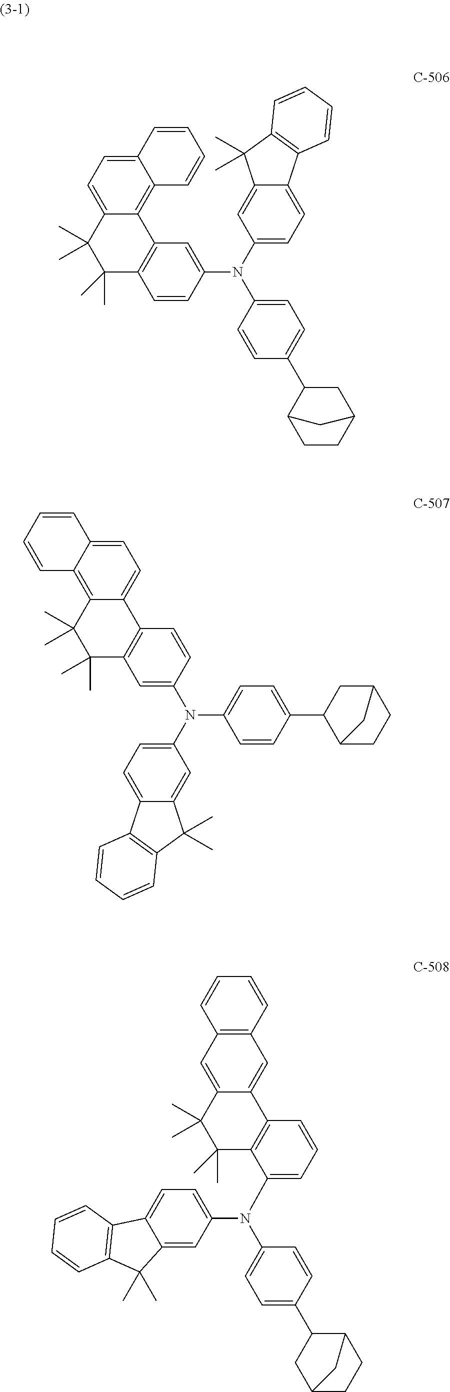 [SMILES: CC1(C)c2ccccc2-c2ccc(N(c3ccc(C4CC5CCC4C5)cc3)c3ccc4c(c3)-c3c(ccc5ccccc35)C(C)(C)C4(C)C)cc21 ; CC1(C)c2ccccc2-c2ccc(N(c3ccc(C4CC5CCC4C5)cc3)c3ccc4c(c3)C(C)(C)C(C)(C)c3c-4ccc4ccccc34)cc21 ; CC1(C)c2ccccc2-c2ccc(N(c3ccc(C4CC5CCC4C5)cc3)c3cccc4c3C(C)(C)C(C)(C)c3cc5ccccc5cc3-4)cc21]

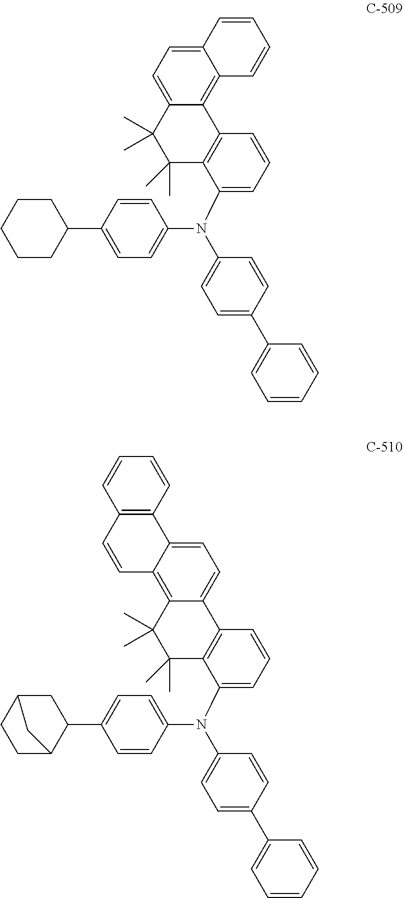 [SMILES: CC1(C)c2ccc3ccccc3c2-c2cccc(N(c3ccc(-c4ccccc4)cc3)c3ccc(C4CCCCC4)cc3)c2C1(C)C ; CC1(C)c2c(cccc2N(c2ccc(-c3ccccc3)cc2)c2ccc(C3CC4CCC3C4)cc2)-c2ccc3c(ccc4ccccc43)c2C1(C)C]

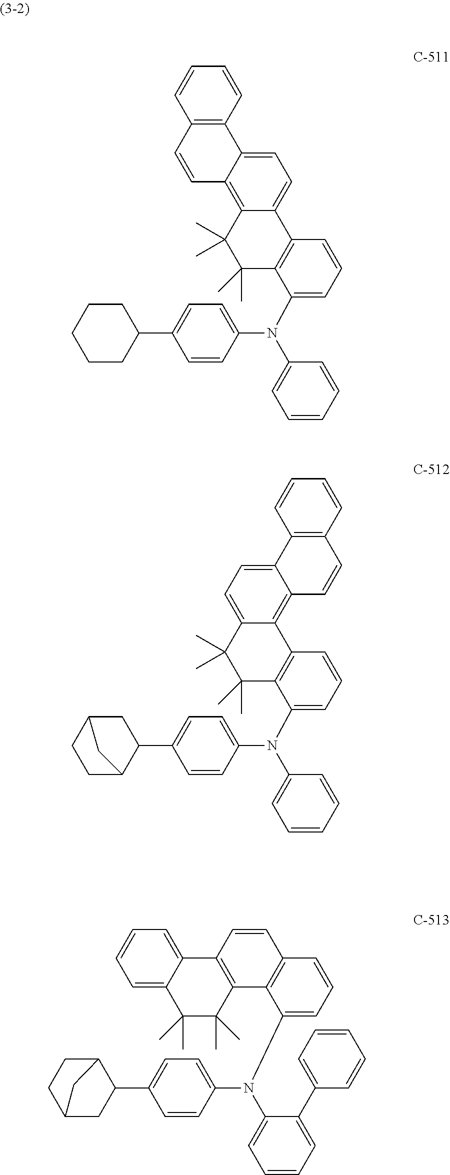 [SMILES: CC1(C)c2c(cccc2N(c2ccccc2)c2ccc(C3CCCCC3)cc2)-c2ccc3c(ccc4ccccc43)c2C1(C)C ; CC1(C)c2ccc3c(ccc4ccccc43)c2-c2cccc(N(c3ccccc3)c3ccc(C4CC5CCC4C5)cc3)c2C1(C)C ; CC1(C)c2ccccc2-c2ccc3cccc(N(c4ccc(C5CC6CCC5C6)cc4)c4ccccc4-c4ccccc4)c3c2C1(C)C]

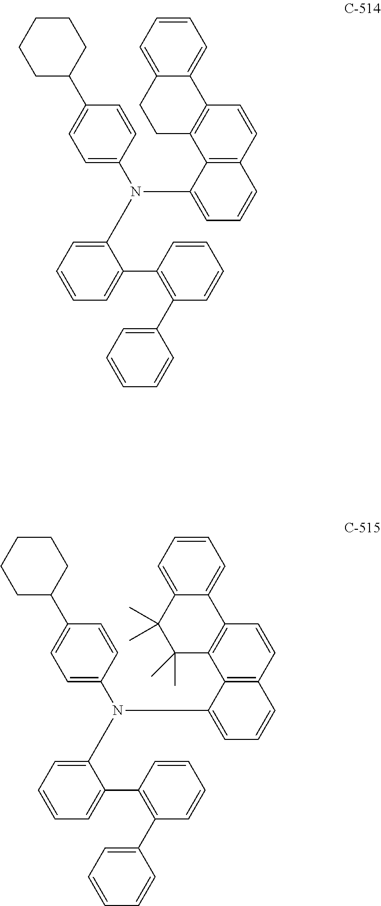 [SMILES: c1ccc(-c2ccccc2-c2ccccc2N(c2ccc(C3CCCCC3)cc2)c2cccc3ccc4c(c23)CCc2ccccc2-4)cc1 ; CC1(C)c2ccccc2-c2ccc3cccc(N(c4ccc(C5CCCCC5)cc4)c4ccccc4-c4ccccc4-c4ccccc4)c3c2C1(C)C]

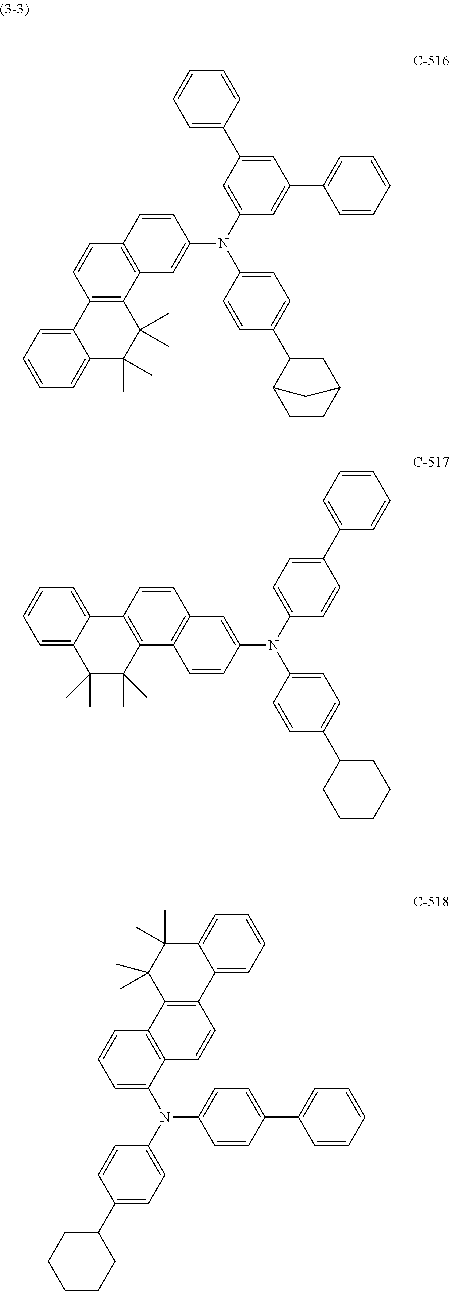 [SMILES: CC1(C)c2ccccc2-c2ccc3ccc(N(c4ccc(C5CC6CCC5C6)cc4)c4cc(-c5ccccc5)cc(-c5ccccc5)c4)cc3c2C1(C)C ; CC1(C)c2ccccc2-c2ccc3cc(N(c4ccc(-c5ccccc5)cc4)c4ccc(C5CCCCC5)cc4)ccc3c2C1(C)C ; CC1(C)c2ccccc2-c2ccc3c(N(c4ccc(-c5ccccc5)cc4)c4ccc(C5CCCCC5)cc4)cccc3c2C1(C)C]

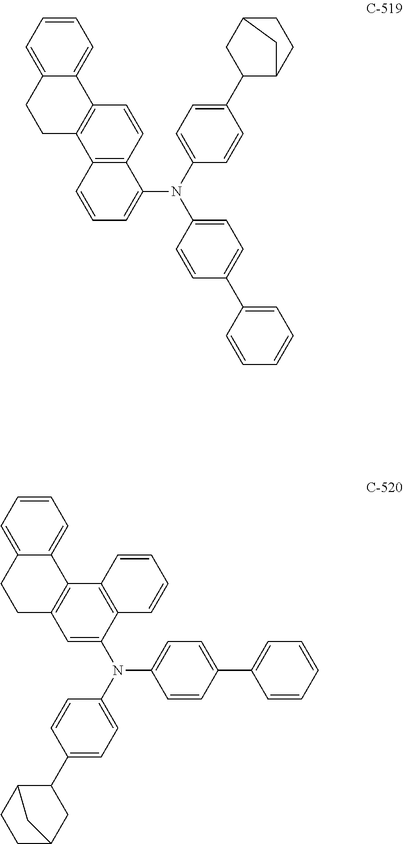 [SMILES: c1ccc(-c2ccc(N(c3ccc(C4CC5CCC4C5)cc3)c3cccc4c5c(ccc34)-c3ccccc3CC5)cc2)cc1 ; c1ccc(-c2ccc(N(c3ccc(C4CC5CCC4C5)cc3)c3cc4c(c5ccccc35)-c3ccccc3CC4)cc2)cc1]

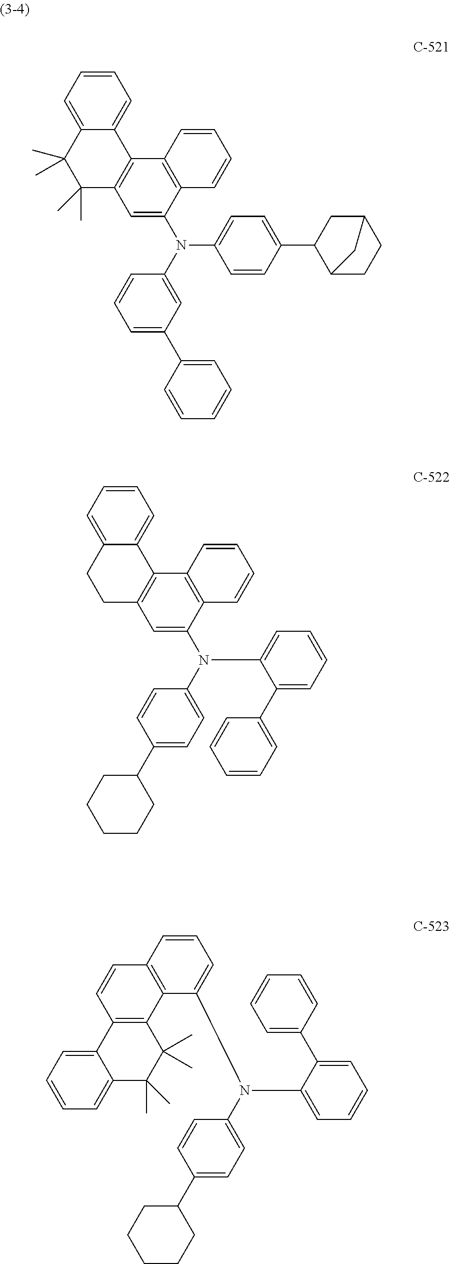 [SMILES: CC1(C)c2ccccc2-c2c(cc(N(c3ccc(C4CC5CCC4C5)cc3)c3cccc(-c4ccccc4)c3)c3ccccc23)C1(C)C ; c1ccc(-c2ccccc2N(c2ccc(C3CCCCC3)cc2)c2cc3c(c4ccccc24)-c2ccccc2CC3)cc1 ; CC1(C)c2ccccc2-c2ccc3cccc(N(c4ccc(C5CCCCC5)cc4)c4ccccc4-c4ccccc4)c3c2C1(C)C]

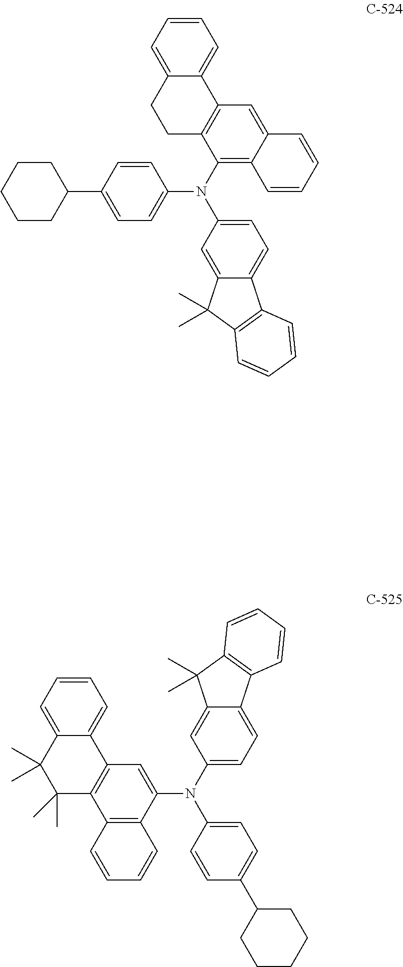 [SMILES: CC1(C)c2ccccc2-c2ccc(N(c3ccc(C4CCCCC4)cc3)c3c4c(cc5ccccc35)-c3ccccc3CC4)cc21 ; CC1(C)c2ccccc2-c2ccc(N(c3ccc(C4CCCCC4)cc3)c3cc4c(c5ccccc35)C(C)(C)C(C)(C)c3ccccc3-4)cc21]

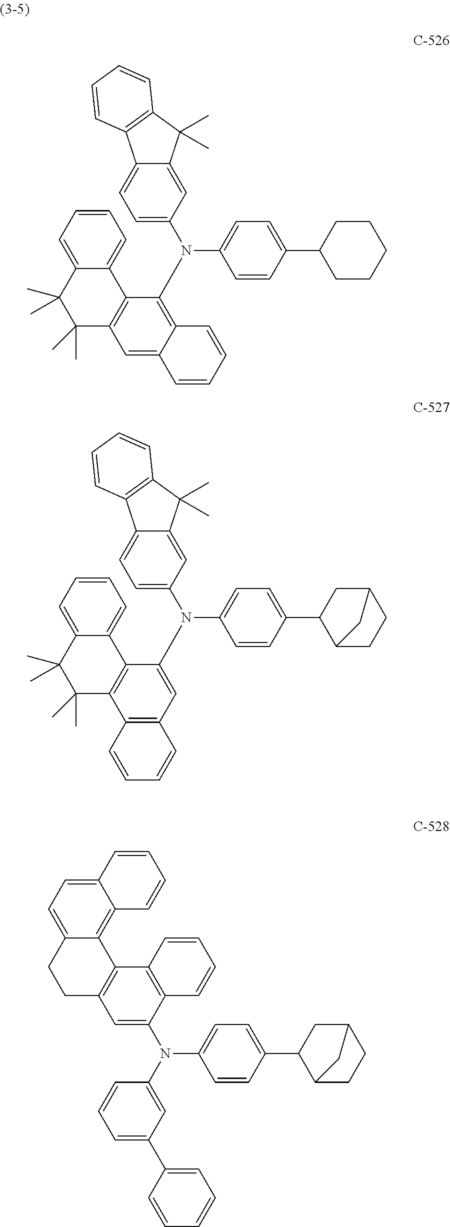 [SMILES: CC1(C)c2ccccc2-c2ccc(N(c3ccc(C4CCCCC4)cc3)c3c4c(cc5ccccc35)C(C)(C)C(C)(C)c3ccccc3-4)cc21 ; CC1(C)c2ccccc2-c2ccc(N(c3ccc(C4CC5CCC4C5)cc3)c3cc4ccccc4c4c3-c3ccccc3C(C)(C)C4(C)C)cc21 ; c1ccc(-c2cccc(N(c3ccc(C4CC5CCC4C5)cc3)c3cc4c(c5ccccc35)-c3c(ccc5ccccc35)CC4)c2)cc1]

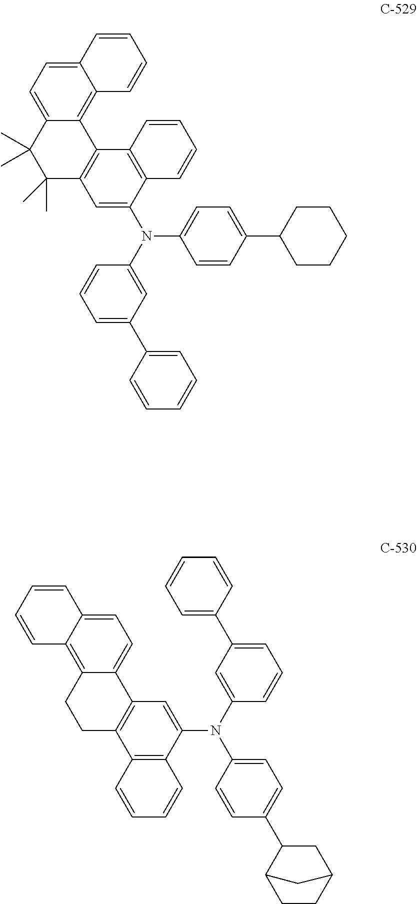 [SMILES: CC1(C)c2ccc3ccccc3c2-c2c(cc(N(c3ccc(C4CCCCC4)cc3)c3cccc(-c4ccccc4)c3)c3ccccc23)C1(C)C ; c1ccc(-c2cccc(N(c3ccc(C4CC5CCC4C5)cc3)c3cc4c(c5ccccc35)CCc3c-4ccc4ccccc34)c2)cc1]

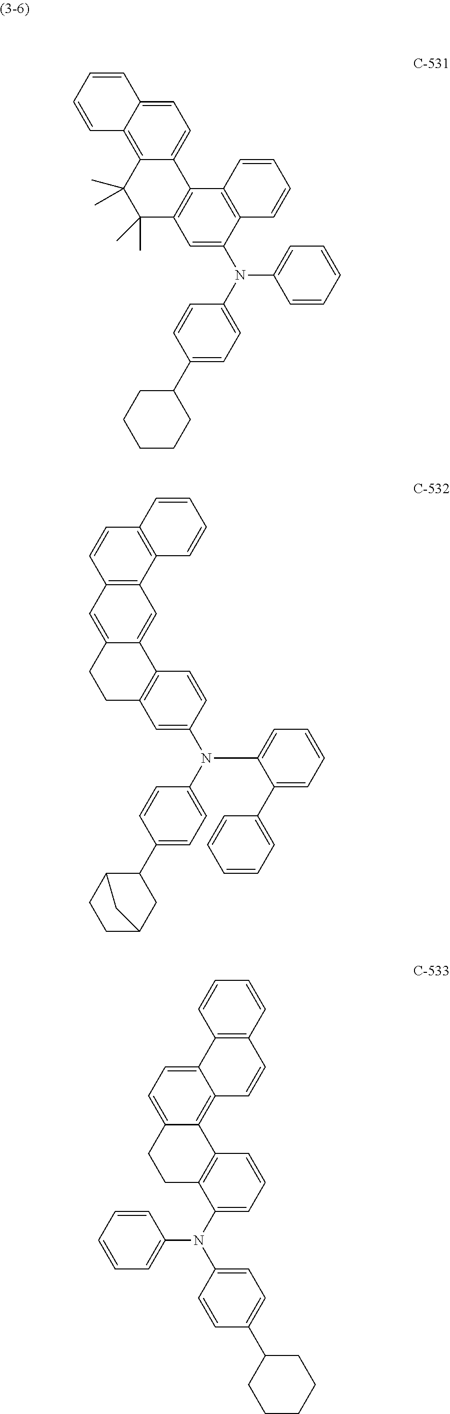 [SMILES: CC1(C)c2cc(N(c3ccccc3)c3ccc(C4CCCCC4)cc3)c3ccccc3c2-c2ccc3ccccc3c2C1(C)C ; c1ccc(-c2ccccc2N(c2ccc(C3CC4CCC3C4)cc2)c2ccc3c(c2)CCc2cc4ccc5ccccc5c4cc2-3)cc1 ; c1ccc(N(c2ccc(C3CCCCC3)cc2)c2cccc3c2CCc2ccc4c(ccc5ccccc54)c2-3)cc1]

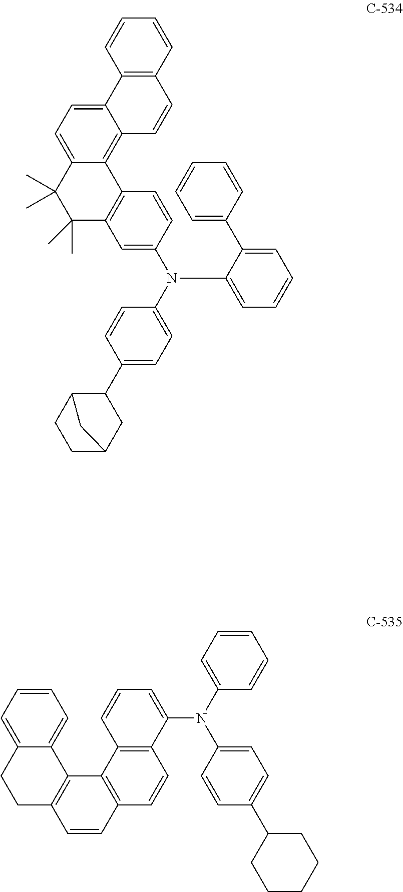 [SMILES: CC1(C)c2cc(N(c3ccc(C4CC5CCC4C5)cc3)c3ccccc3-c3ccccc3)ccc2-c2c(ccc3c2ccc2ccccc23)C1(C)C ; c1ccc(N(c2ccc(C3CCCCC3)cc2)c2cccc3c2ccc2ccc4c(c23)-c2ccccc2CC4)cc1]

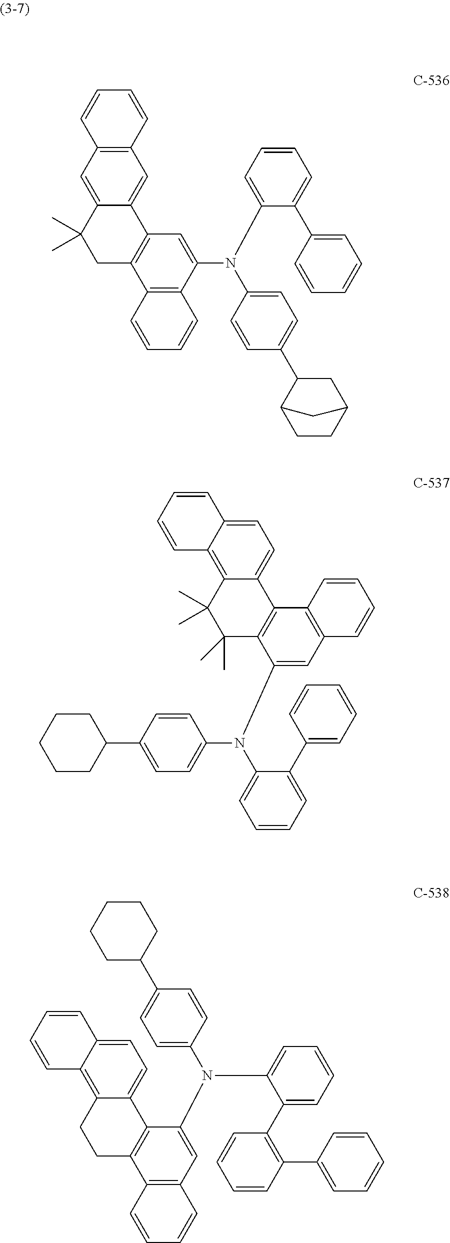 [SMILES: CC1(C)Cc2c(cc(N(c3ccc(C4CC5CCC4C5)cc3)c3ccccc3-c3ccccc3)c3ccccc23)-c2cc3ccccc3cc21 ; CC1(C)c2c(N(c3ccc(C4CCCCC4)cc3)c3ccccc3-c3ccccc3)cc3ccccc3c2-c2ccc3ccccc3c2C1(C)C ; c1ccc(-c2ccccc2-c2ccccc2N(c2ccc(C3CCCCC3)cc2)c2cc3ccccc3c3c2-c2ccc4ccccc4c2CC3)cc1]

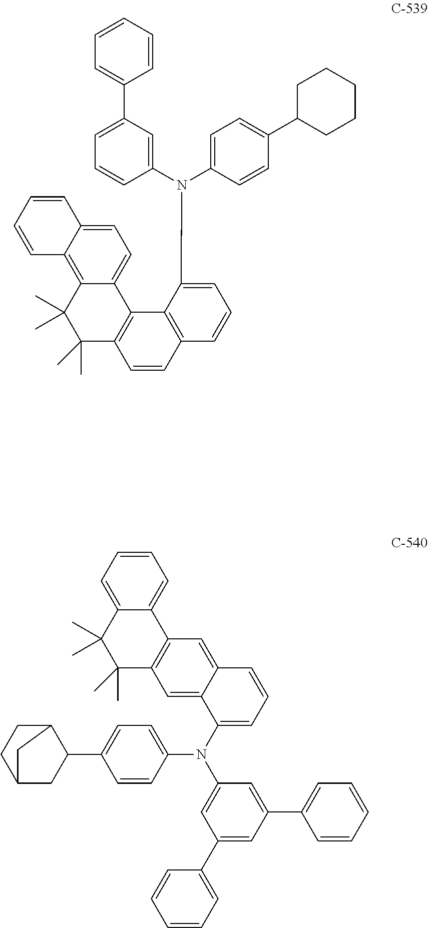 [SMILES: CC1(C)c2ccc3cccc(N(c4ccc(C5CCCCC5)cc4)c4cccc(-c5ccccc5)c4)c3c2-c2ccc3ccccc3c2C1(C)C ; CC1(C)c2ccccc2-c2cc3cccc(N(c4ccc(C5CC6CCC5C6)cc4)c4cc(-c5ccccc5)cc(-c5ccccc5)c4)c3cc2C1(C)C]

In Formulas 3-1 to 3-7,
R₁ to R₄ are as defined in Formula 1; and
R₅ to R₁₆, R₂₁ to R₂₄ and R_{c} to R_{f} are as defined for Rₐ in Formula 1;
provided that at least one of R₁ to R₁₆, R₂₁ to R₂₄, and R_{c} to R_{f} is represented by Formula 1-1.

According to one embodiment, the organic electroluminescent compound represented by Formula 1 may be represented by any one of the following Formulas 4-1 to 4-6.

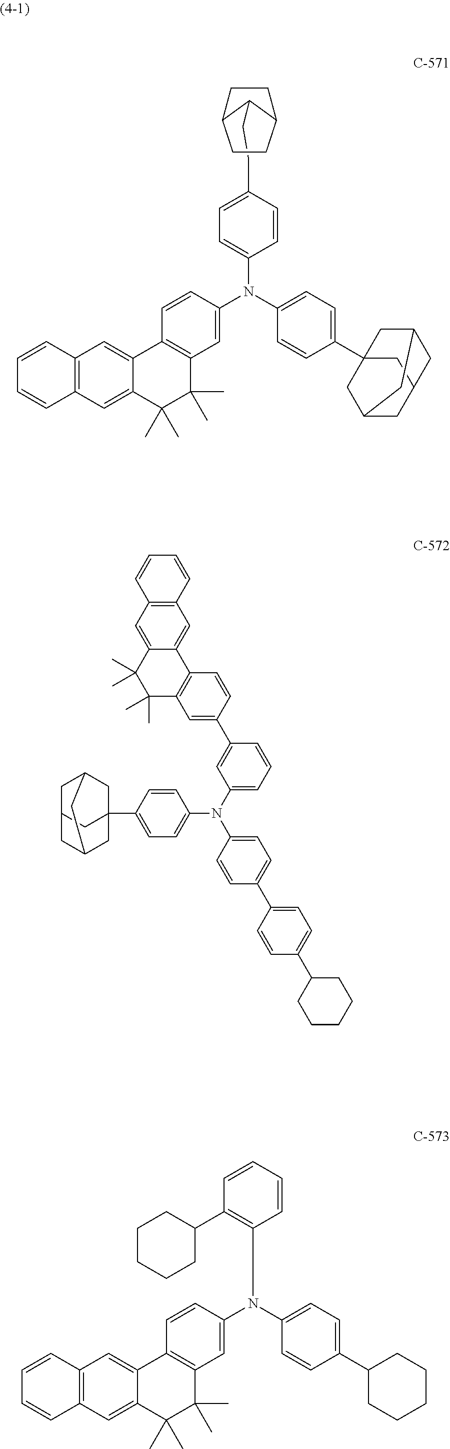 [SMILES: CC1(C)c2cc(N(c3ccc(CCC4C5CCC4CC5)cc3)c3ccc(C45CC6CC(CC(C6)C4)C5)cc3)ccc2-c2cc3ccccc3cc2C1(C)C ; CC1(C)c2cc(-c3cccc(N(c4ccc(-c5ccc(C6CCCCC6)cc5)cc4)c4ccc(C56CC7CC(CC(C7)C5)C6)cc4)c3)ccc2-c2cc3ccccc3cc2C1(C)C ; CC1(C)c2cc(N(c3ccc(C4CCCCC4)cc3)c3ccccc3C3CCCCC3)ccc2-c2cc3ccccc3cc2C1(C)C]

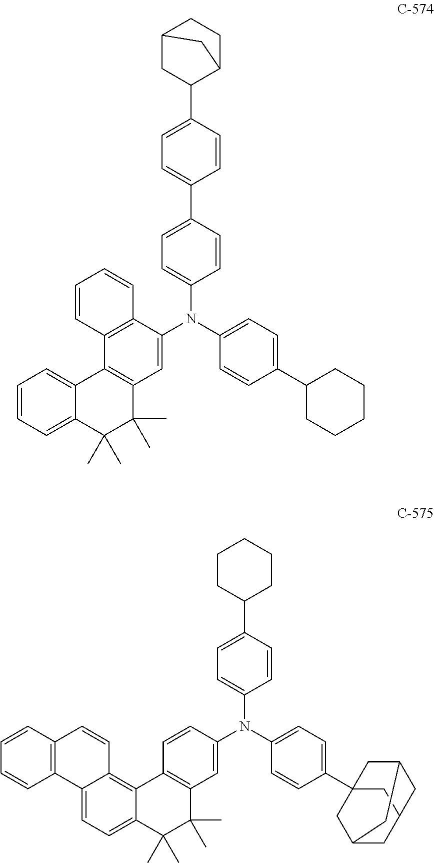 [SMILES: CC1(C)c2ccccc2-c2c(cc(N(c3ccc(-c4ccc(C5CC6CCC5C6)cc4)cc3)c3ccc(C4CCCCC4)cc3)c3ccccc23)C1(C)C ; CC1(C)c2cc(N(c3ccc(C4CCCCC4)cc3)c3ccc(C45CC6CC(CC(C6)C4)C5)cc3)ccc2-c2c(ccc3c2ccc2ccccc23)C1(C)C]

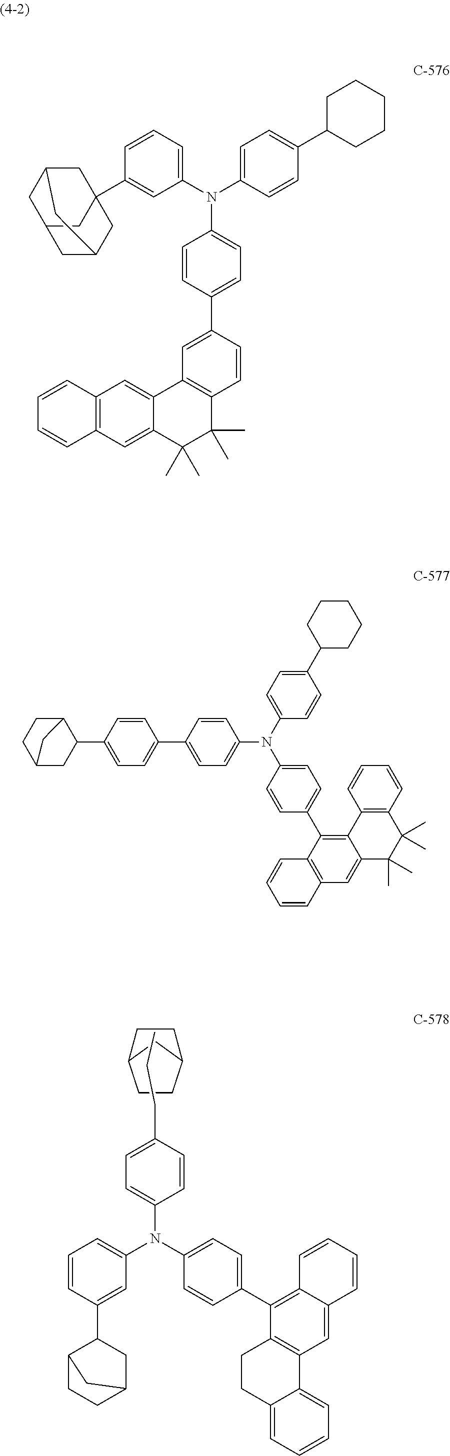

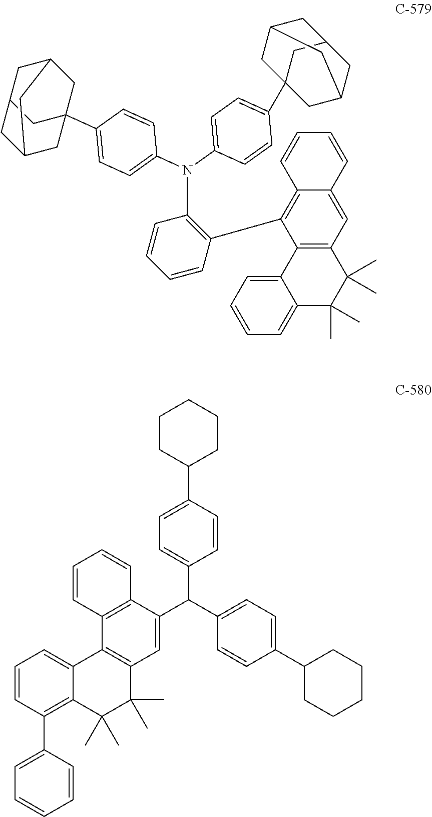

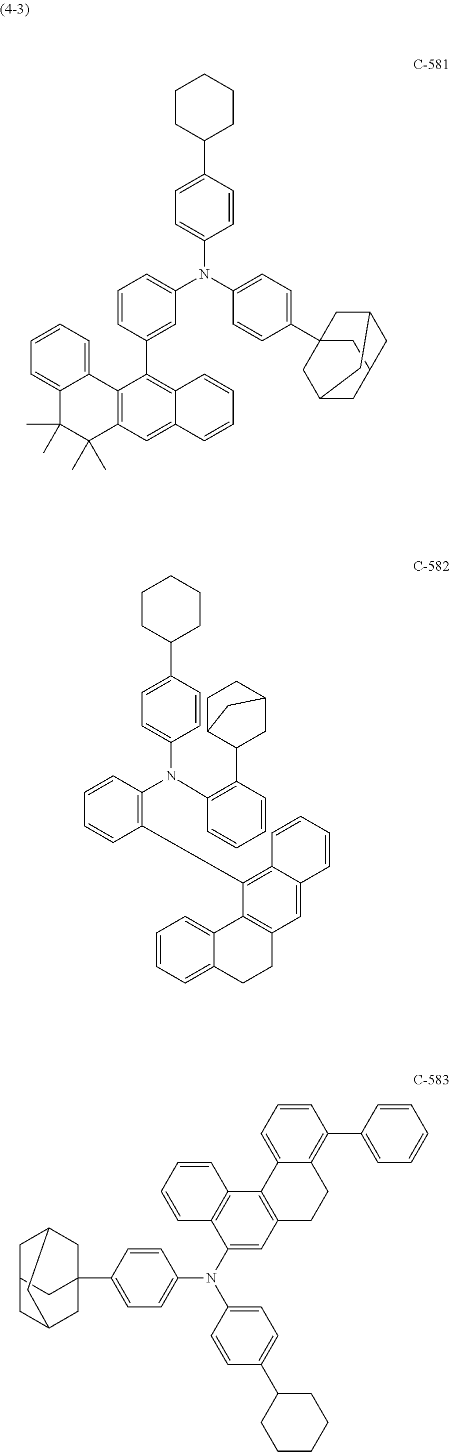 [SMILES: CC1(C)c2ccccc2-c2c(cc3ccccc3c2-c2cccc(N(c3ccc(C4CCCCC4)cc3)c3ccc(C45CC6CC(CC(C6)C4)C5)cc3)c2)C1(C)C ; c1ccc2c(c1)CCc1cc3ccccc3c(-c3ccccc3N(c3ccc(C4CCCCC4)cc3)c3ccccc3C3CC4CCC3C4)c1-2 ; c1ccc(-c2cccc3c2CCc2cc(N(c4ccc(C5CCCCC5)cc4)c4ccc(C56CC7CC(CC(C7)C5)C6)cc4)c4ccccc4c2-3)cc1]

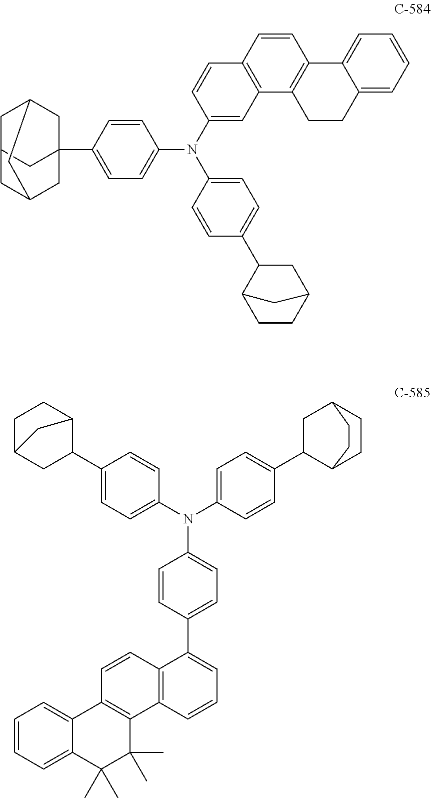 [SMILES: c1ccc2c(c1)CCc1c-2ccc2ccc(N(c3ccc(C4CC5CCC4C5)cc3)c3ccc(C45CC6CC(CC(C6)C4)C5)cc3)cc12 ; CC1(C)c2ccccc2-c2ccc3c(-c4ccc(N(c5ccc(C6CC7CCC6CC7)cc5)c5ccc(C6CC7CCC6C7)cc5)cc4)cccc3c2C1(C)C]

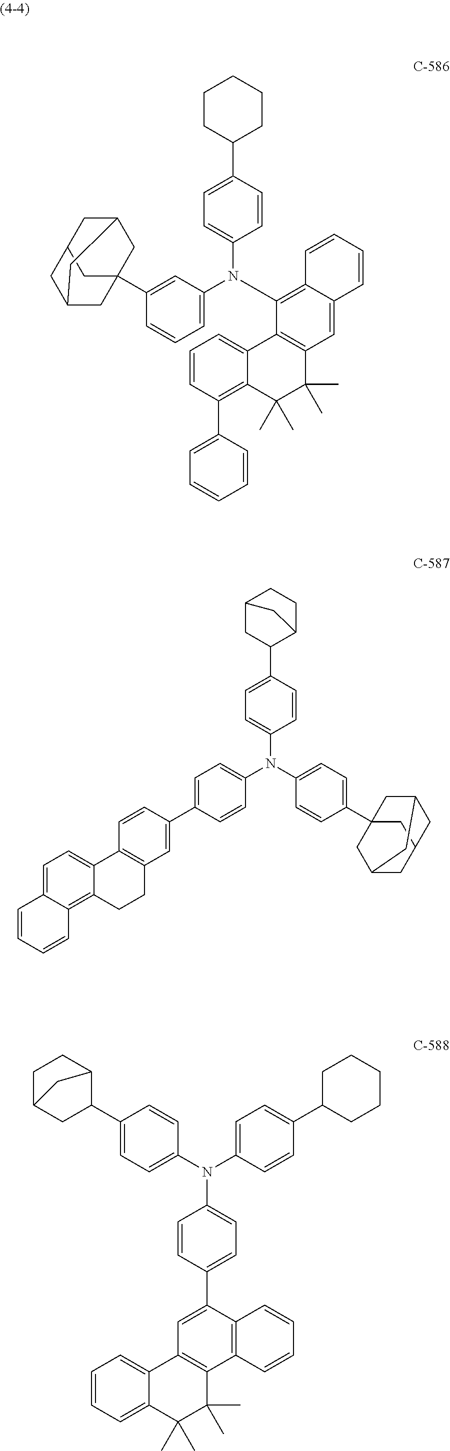 [SMILES: CC1(C)c2cc3ccccc3c(N(c3ccc(C4CCCCC4)cc3)c3cccc(C45CC6CC(CC(C6)C4)C5)c3)c2-c2cccc(-c3ccccc3)c2C1(C)C ; c1ccc2c3c(ccc2c1)-c1ccc(-c2ccc(N(c4ccc(C5CC6CCC5C6)cc4)c4ccc(C56CC7CC(CC(C7)C5)C6)cc4)cc2)cc1CC3 ; CC1(C)c2ccccc2-c2cc(-c3ccc(N(c4ccc(C5CCCCC5)cc4)c4ccc(C5CC6CCC5C6)cc4)cc3)c3ccccc3c2C1(C)C]

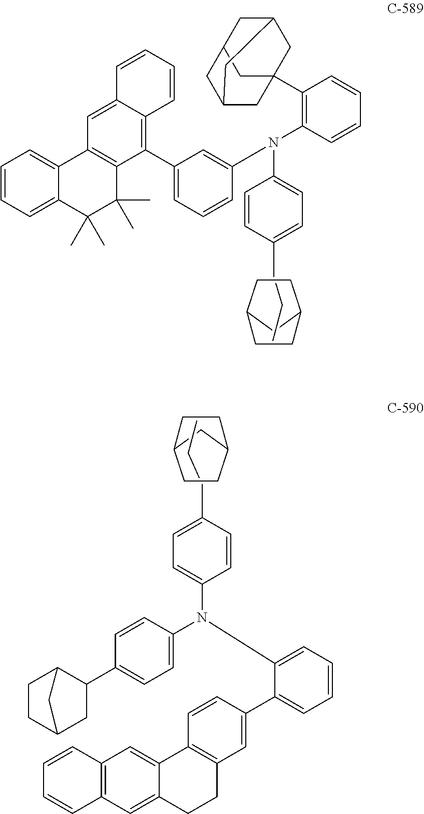

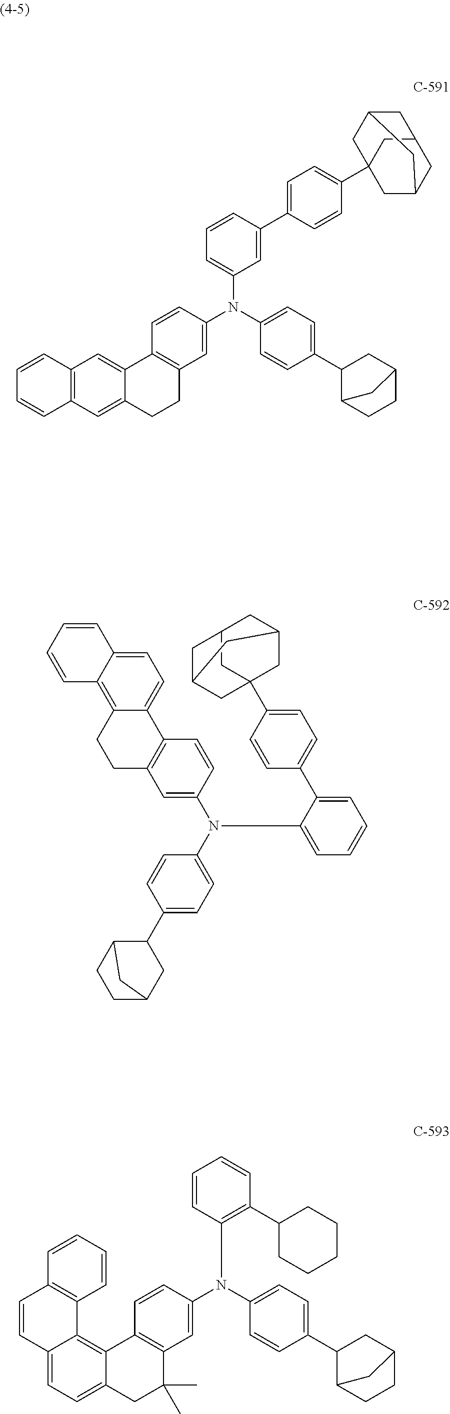 [SMILES: c1cc(-c2ccc(C34CC5CC(CC(C5)C3)C4)cc2)cc(N(c2ccc(C3CC4CCC3C4)cc2)c2ccc3c(c2)CCc2cc4ccccc4cc2-3)c1 ; c1ccc(N(c2ccc(C3CC4CCC3C4)cc2)c2ccc3c(c2)CCc2c-3ccc3ccccc23)c(-c2ccc(C34CC5CC(CC(C5)C3)C4)cc2)c1 ; CC1(C)Cc2ccc3ccc4ccccc4c3c2-c2ccc(N(c3ccc(C4CC5CCC4C5)cc3)c3ccccc3C3CCCCC3)cc21]

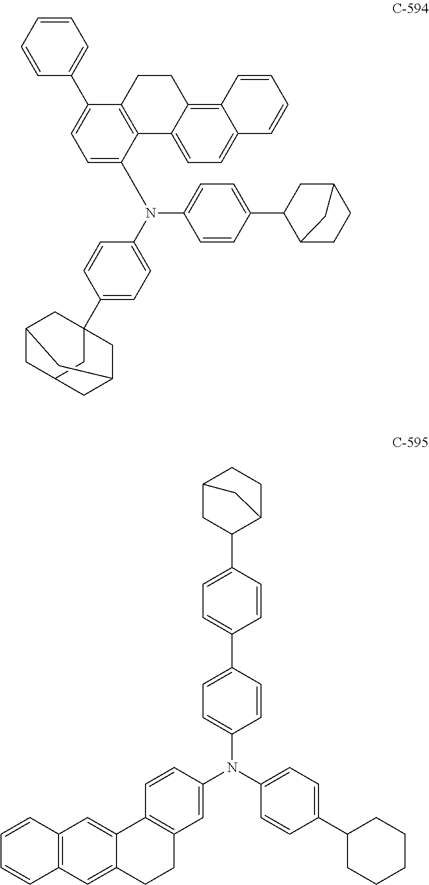 [SMILES: c1ccc(-c2ccc(N(c3ccc(C4CC5CCC4C5)cc3)c3ccc(C45CC6CC(CC(C6)C4)C5)cc3)c3c2CCc2c-3ccc3ccccc23)cc1 ; c1ccc2cc3c(cc2c1)CCc1cc(N(c2ccc(-c4ccc(C5CC6CCC5C6)cc4)cc2)c2ccc(C4CCCCC4)cc2)ccc1-3]

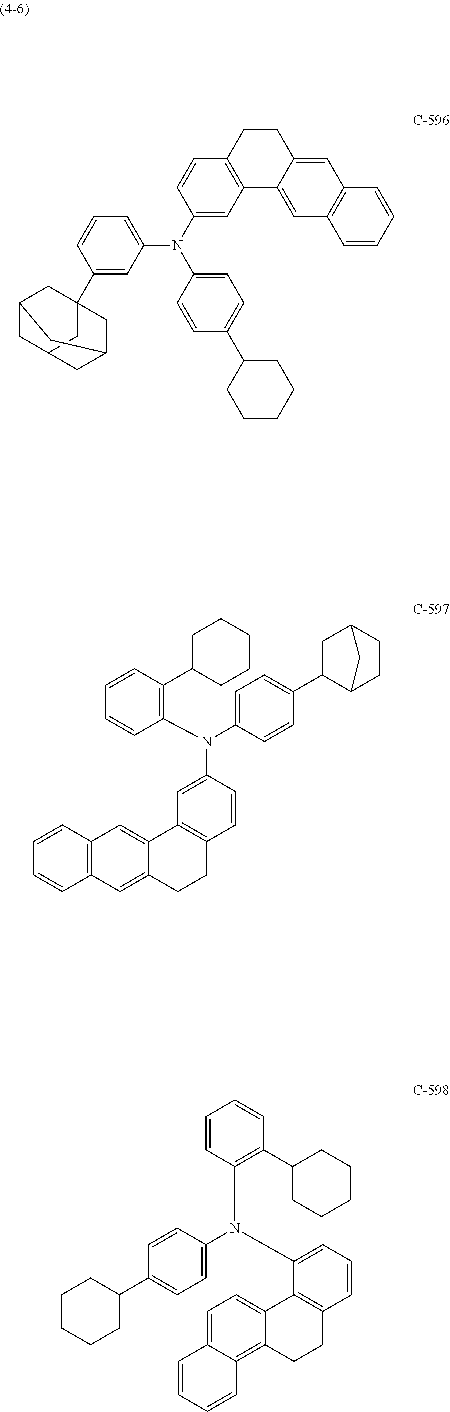 [SMILES: c1cc(N(c2ccc(C3CCCCC3)cc2)c2ccc3c(c2)-c2cc4ccccc4cc2CC3)cc(C23CC4CC(CC(C4)C2)C3)c1 ; c1ccc(N(c2ccc(C3CC4CCC3C4)cc2)c2ccc3c(c2)-c2cc4ccccc4cc2CC3)c(C2CCCCC2)c1 ; c1ccc(N(c2ccc(C3CCCCC3)cc2)c2cccc3c2-c2ccc4ccccc4c2CC3)c(C2CCCCC2)c1]

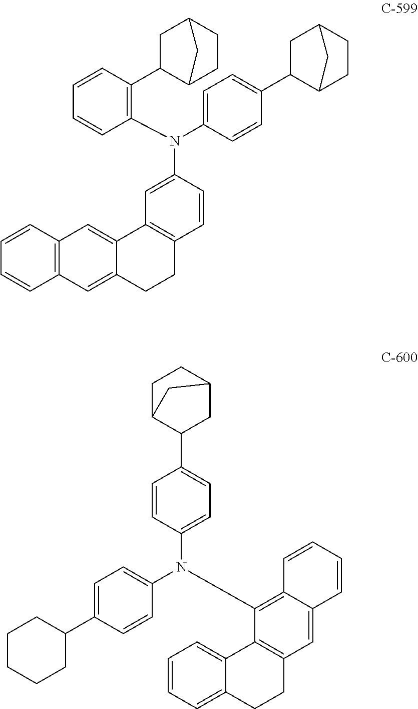 [SMILES: c1ccc(N(c2ccc(C3CC4CCC3C4)cc2)c2ccc3c(c2)-c2cc4ccccc4cc2CC3)c(C2CC3CCC2C3)c1 ; c1ccc2c(c1)CCc1cc3ccccc3c(N(c3ccc(C4CCCCC4)cc3)c3ccc(C4CC5CCC4C5)cc3)c1-2]

In Formulas 4-1 to 4-6,
X represents NR₂₉, O, S, or CR₃₀R₃₁;
R₁ to R₄ are as defined in Formula 1; and
R₅ to R₁₂ and R₂₅ to R₃₁ are as defined for Rₐ in Formula 1;
provided that at least one of R₁ to R₁₂ and R₂₅ to R₃₁ is represented by Formula 1-1.

According to one embodiment, an organic electroluminescent compound represented by Formula 1 may be more specifically illustrated by the following compounds, but is not limited thereto.

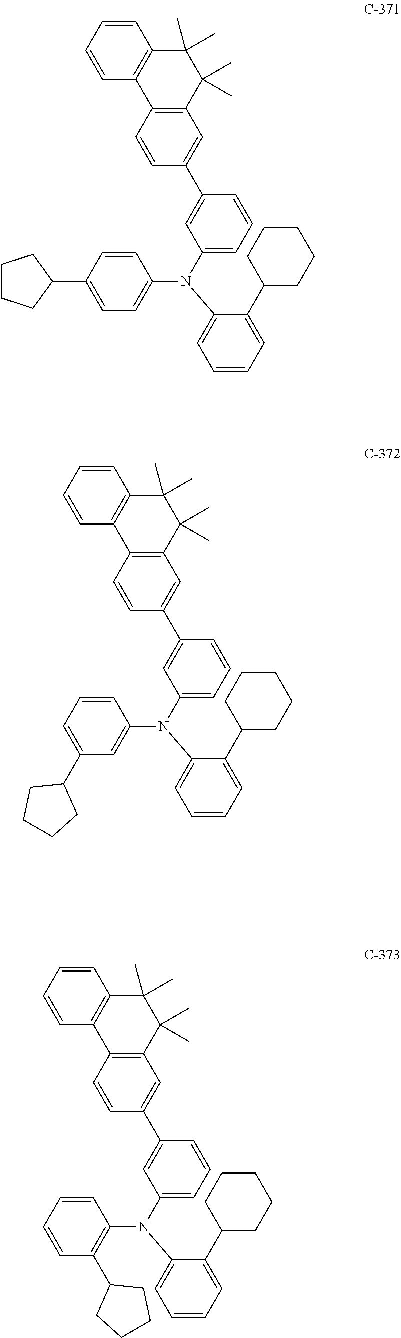 [SMILES: CC1(C)c2ccccc2-c2ccc(-c3cccc(N(c4ccc(C5CCCC5)cc4)c4ccccc4C4CCCCC4)c3)cc2C1(C)C ; CC1(C)c2ccccc2-c2ccc(-c3cccc(N(c4cccc(C5CCCC5)c4)c4ccccc4C4CCCCC4)c3)cc2C1(C)C ; CC1(C)c2ccccc2-c2ccc(-c3cccc(N(c4ccccc4C4CCCCC4)c4ccccc4C4CCCC4)c3)cc2C1(C)C]

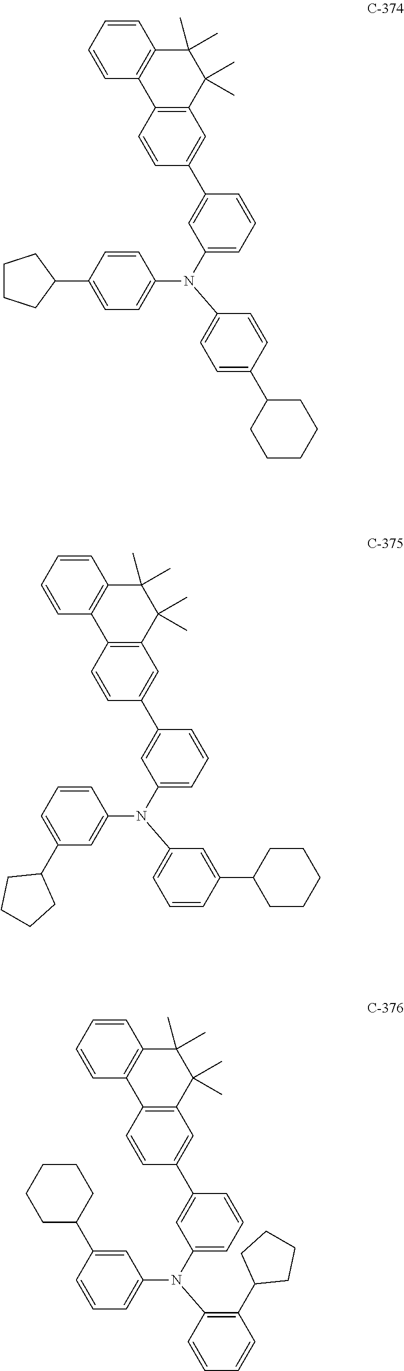 [SMILES: CC1(C)c2ccccc2-c2ccc(-c3cccc(N(c4ccc(C5CCCCC5)cc4)c4ccc(C5CCCC5)cc4)c3)cc2C1(C)C ; CC1(C)c2ccccc2-c2ccc(-c3cccc(N(c4cccc(C5CCCCC5)c4)c4cccc(C5CCCC5)c4)c3)cc2C1(C)C ; CC1(C)c2ccccc2-c2ccc(-c3cccc(N(c4cccc(C5CCCCC5)c4)c4ccccc4C4CCCC4)c3)cc2C1(C)C]

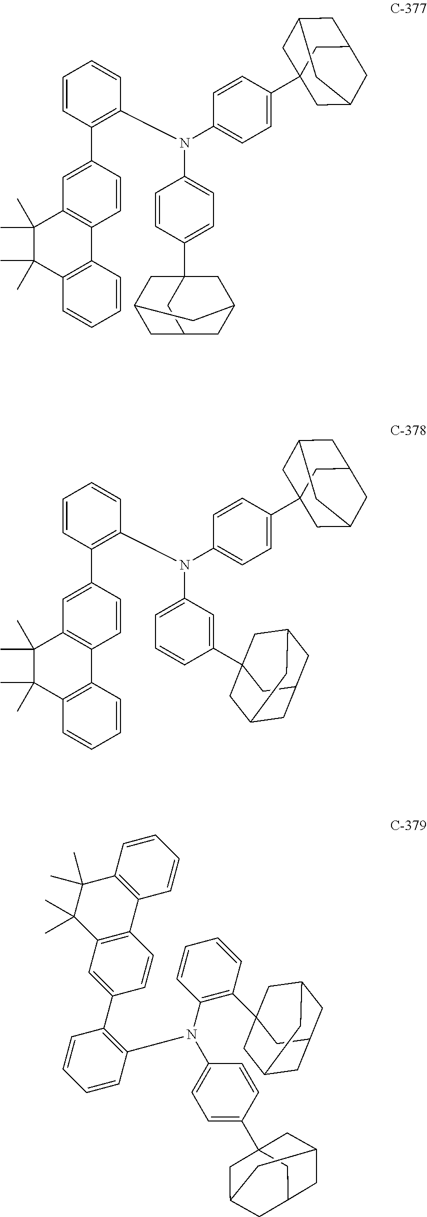 [SMILES: CC1(C)c2ccccc2-c2ccc(-c3ccccc3N(c3ccc(C45CC6CC(CC(C6)C4)C5)cc3)c3ccc(C45CC6CC(CC(C6)C4)C5)cc3)cc2C1(C)C ; CC1(C)c2ccccc2-c2ccc(-c3ccccc3N(c3ccc(C45CC6CC(CC(C6)C4)C5)cc3)c3cccc(C45CC6CC(CC(C6)C4)C5)c3)cc2C1(C)C ; CC1(C)c2ccccc2-c2ccc(-c3ccccc3N(c3ccc(C45CC6CC(CC(C6)C4)C5)cc3)c3ccccc3C34CC5CC(CC(C5)C3)C4)cc2C1(C)C]

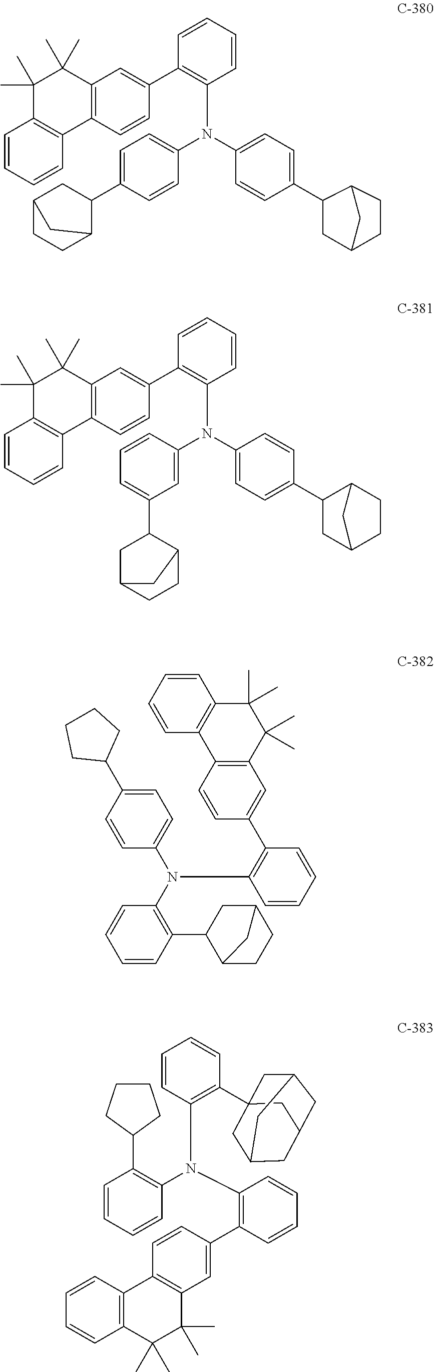 [SMILES: CC1(C)c2ccccc2-c2ccc(-c3ccccc3N(c3ccc(C4CC5CCC4C5)cc3)c3ccc(C4CC5CCC4C5)cc3)cc2C1(C)C ; CC1(C)c2ccccc2-c2ccc(-c3ccccc3N(c3ccc(C4CC5CCC4C5)cc3)c3cccc(C4CC5CCC4C5)c3)cc2C1(C)C ; CC1(C)c2ccccc2-c2ccc(-c3ccccc3N(c3ccc(C4CCCC4)cc3)c3ccccc3C3CC4CCC3C4)cc2C1(C)C ; CC1(C)c2ccccc2-c2ccc(-c3ccccc3N(c3ccccc3C3CCCC3)c3ccccc3C34CC5CC(CC(C5)C3)C4)cc2C1(C)C]

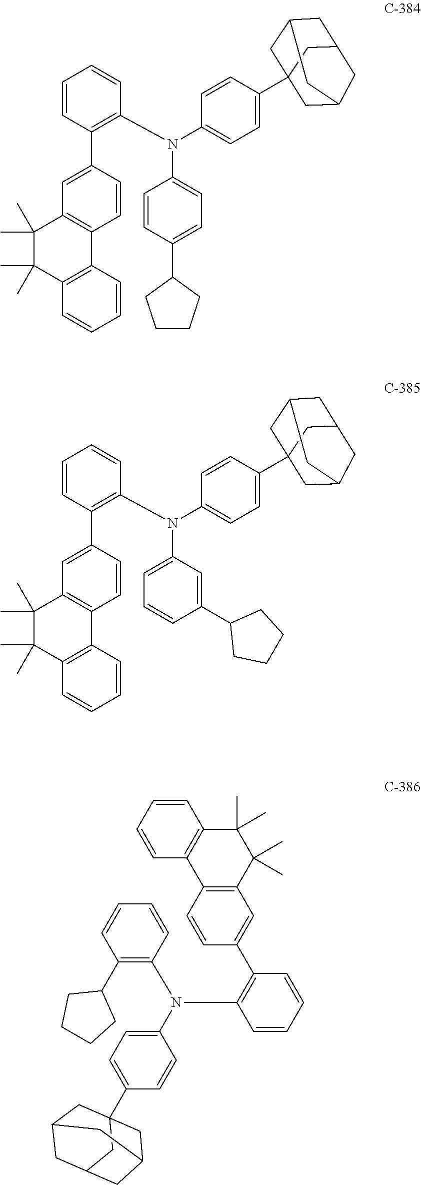 [SMILES: CC1(C)c2ccccc2-c2ccc(-c3ccccc3N(c3ccc(C4CCCC4)cc3)c3ccc(C45CC6CC(CC(C6)C4)C5)cc3)cc2C1(C)C ; CC1(C)c2ccccc2-c2ccc(-c3ccccc3N(c3ccc(C45CC6CC(CC(C6)C4)C5)cc3)c3cccc(C4CCCC4)c3)cc2C1(C)C ; CC1(C)c2ccccc2-c2ccc(-c3ccccc3N(c3ccc(C45CC6CC(CC(C6)C4)C5)cc3)c3ccccc3C3CCCC3)cc2C1(C)C]

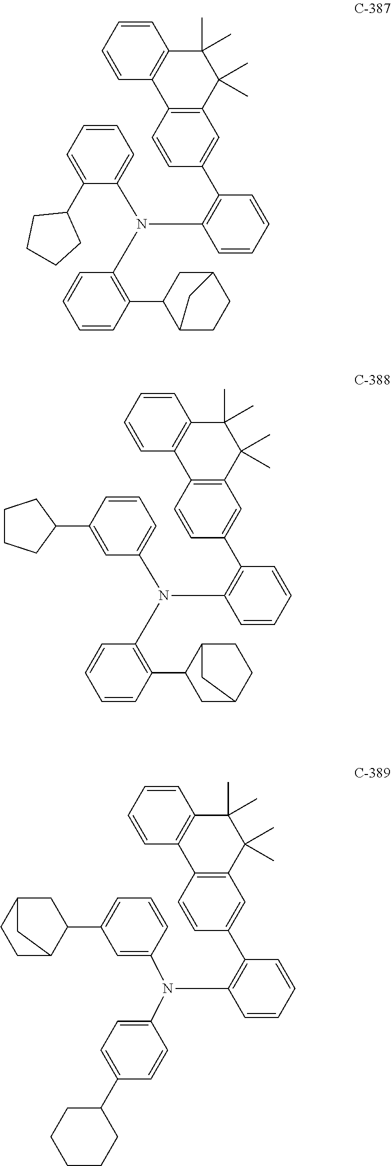 [SMILES: CC1(C)c2ccccc2-c2ccc(-c3ccccc3N(c3ccccc3C3CCCC3)c3ccccc3C3CC4CCC3C4)cc2C1(C)C ; CC1(C)c2ccccc2-c2ccc(-c3ccccc3N(c3cccc(C4CCCC4)c3)c3ccccc3C3CC4CCC3C4)cc2C1(C)C ; CC1(C)c2ccccc2-c2ccc(-c3ccccc3N(c3ccc(C4CCCCC4)cc3)c3cccc(C4CC5CCC4C5)c3)cc2C1(C)C]

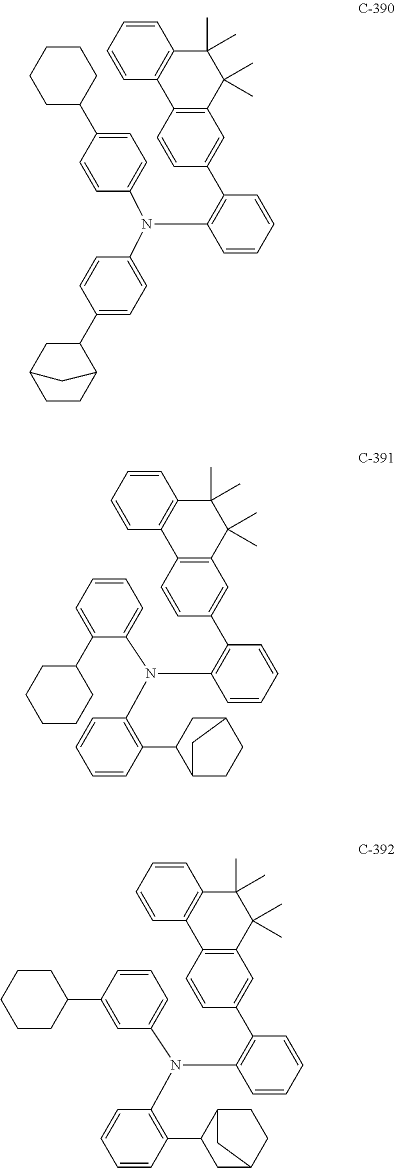 [SMILES: CC1(C)c2ccccc2-c2ccc(-c3ccccc3N(c3ccc(C4CCCCC4)cc3)c3ccc(C4CC5CCC4C5)cc3)cc2C1(C)C ; CC1(C)c2ccccc2-c2ccc(-c3ccccc3N(c3ccccc3C3CCCCC3)c3ccccc3C3CC4CCC3C4)cc2C1(C)C ; CC1(C)c2ccccc2-c2ccc(-c3ccccc3N(c3cccc(C4CCCCC4)c3)c3ccccc3C3CC4CCC3C4)cc2C1(C)C]

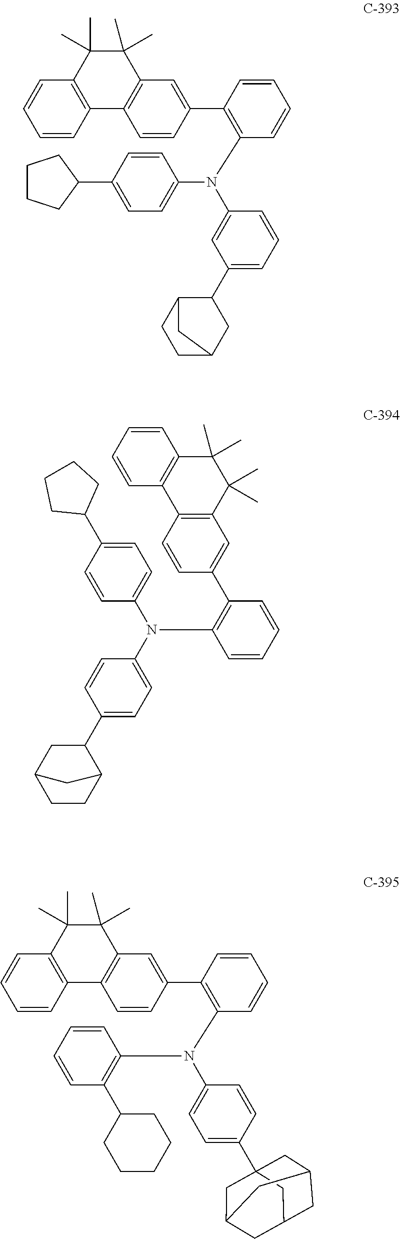 [SMILES: CC1(C)c2ccccc2-c2ccc(-c3ccccc3N(c3ccc(C4CCCC4)cc3)c3cccc(C4CC5CCC4C5)c3)cc2C1(C)C ; CC1(C)c2ccccc2-c2ccc(-c3ccccc3N(c3ccc(C4CCCC4)cc3)c3ccc(C4CC5CCC4C5)cc3)cc2C1(C)C ; CC1(C)c2ccccc2-c2ccc(-c3ccccc3N(c3ccc(C45CC6CC(CC(C6)C4)C5)cc3)c3ccccc3C3CCCCC3)cc2C1(C)C]

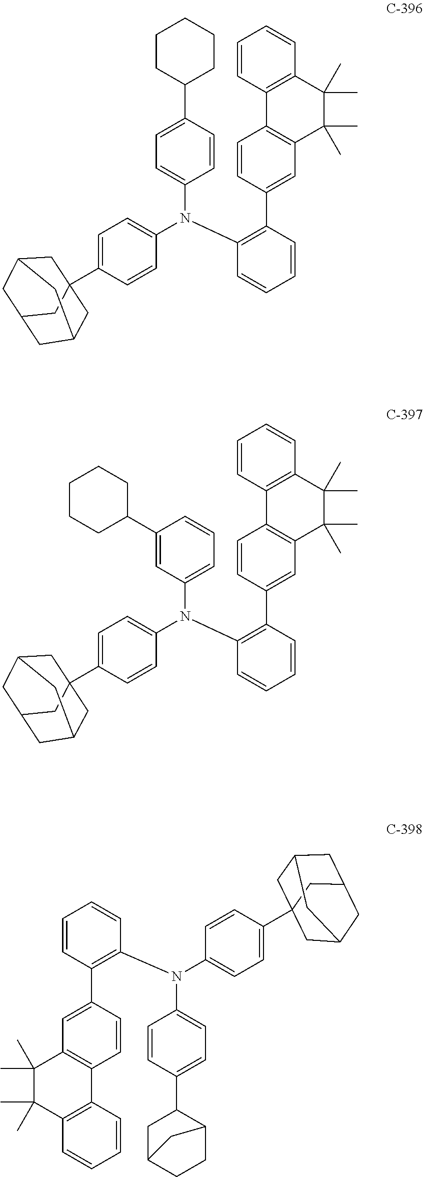

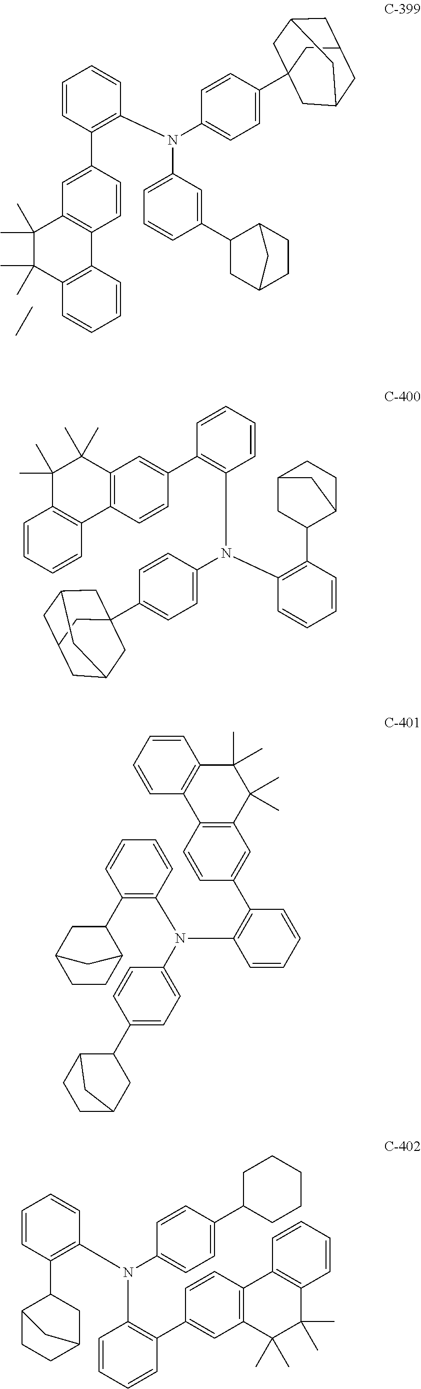

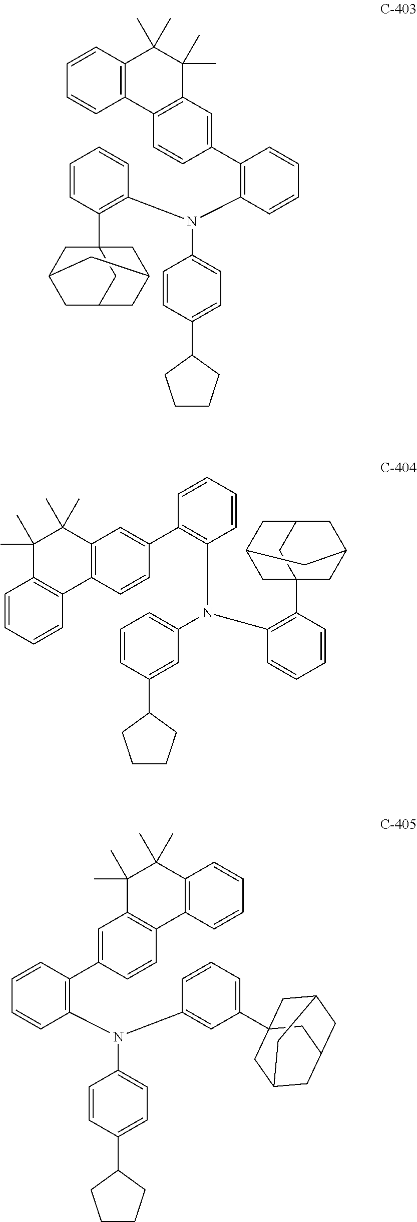 [SMILES: CC1(C)c2ccccc2-c2ccc(-c3ccccc3N(c3ccc(C4CCCC4)cc3)c3ccccc3C34CC5CC(CC(C5)C3)C4)cc2C1(C)C ; CC1(C)c2ccccc2-c2ccc(-c3ccccc3N(c3cccc(C4CCCC4)c3)c3ccccc3C34CC5CC(CC(C5)C3)C4)cc2C1(C)C ; CC1(C)c2ccccc2-c2ccc(-c3ccccc3N(c3ccc(C4CCCC4)cc3)c3cccc(C45CC6CC(CC(C6)C4)C5)c3)cc2C1(C)C]

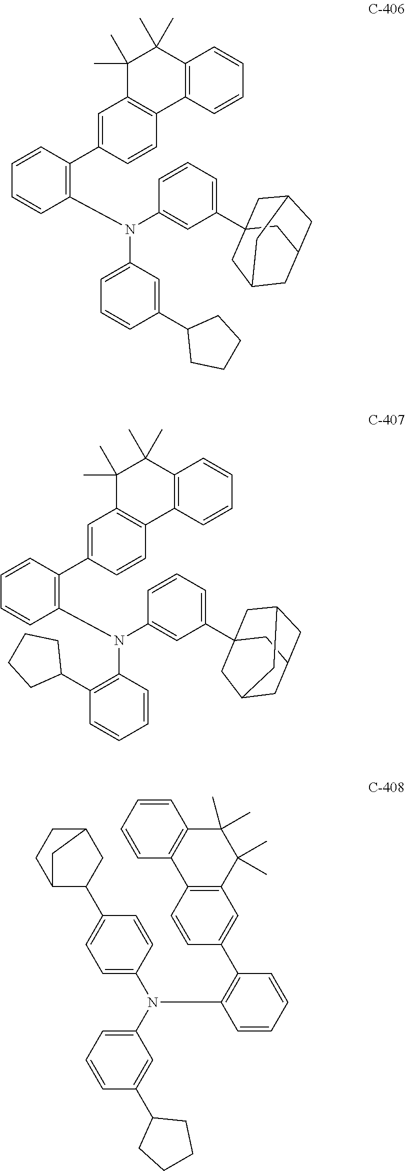 [SMILES: CC1(C)c2ccccc2-c2ccc(-c3ccccc3N(c3cccc(C4CCCC4)c3)c3cccc(C45CC6CC(CC(C6)C4)C5)c3)cc2C1(C)C ; CC1(C)c2ccccc2-c2ccc(-c3ccccc3N(c3cccc(C45CC6CC(CC(C6)C4)C5)c3)c3ccccc3C3CCCC3)cc2C1(C)C ; CC1(C)c2ccccc2-c2ccc(-c3ccccc3N(c3ccc(C4CC5CCC4C5)cc3)c3cccc(C4CCCC4)c3)cc2C1(C)C]

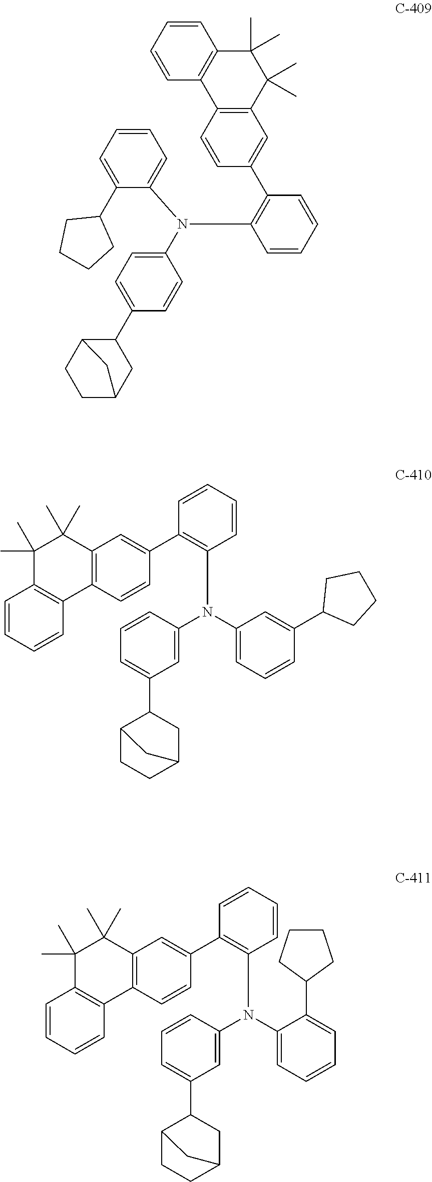 [SMILES: CC1(C)c2ccccc2-c2ccc(-c3ccccc3N(c3ccc(C4CC5CCC4C5)cc3)c3ccccc3C3CCCC3)cc2C1(C)C ; CC1(C)c2ccccc2-c2ccc(-c3ccccc3N(c3cccc(C4CCCC4)c3)c3cccc(C4CC5CCC4C5)c3)cc2C1(C)C ; CC1(C)c2ccccc2-c2ccc(-c3ccccc3N(c3cccc(C4CC5CCC4C5)c3)c3ccccc3C3CCCC3)cc2C1(C)C]

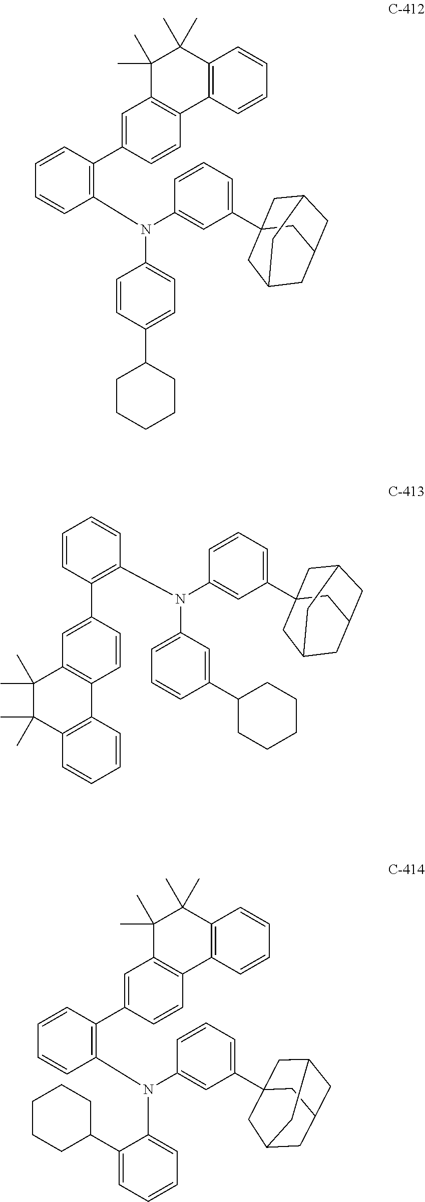 [SMILES: CC1(C)c2ccccc2-c2ccc(-c3ccccc3N(c3ccc(C4CCCCC4)cc3)c3cccc(C45CC6CC(CC(C6)C4)C5)c3)cc2C1(C)C ; CC1(C)c2ccccc2-c2ccc(-c3ccccc3N(c3cccc(C4CCCCC4)c3)c3cccc(C45CC6CC(CC(C6)C4)C5)c3)cc2C1(C)C ; CC1(C)c2ccccc2-c2ccc(-c3ccccc3N(c3cccc(C45CC6CC(CC(C6)C4)C5)c3)c3ccccc3C3CCCCC3)cc2C1(C)C]

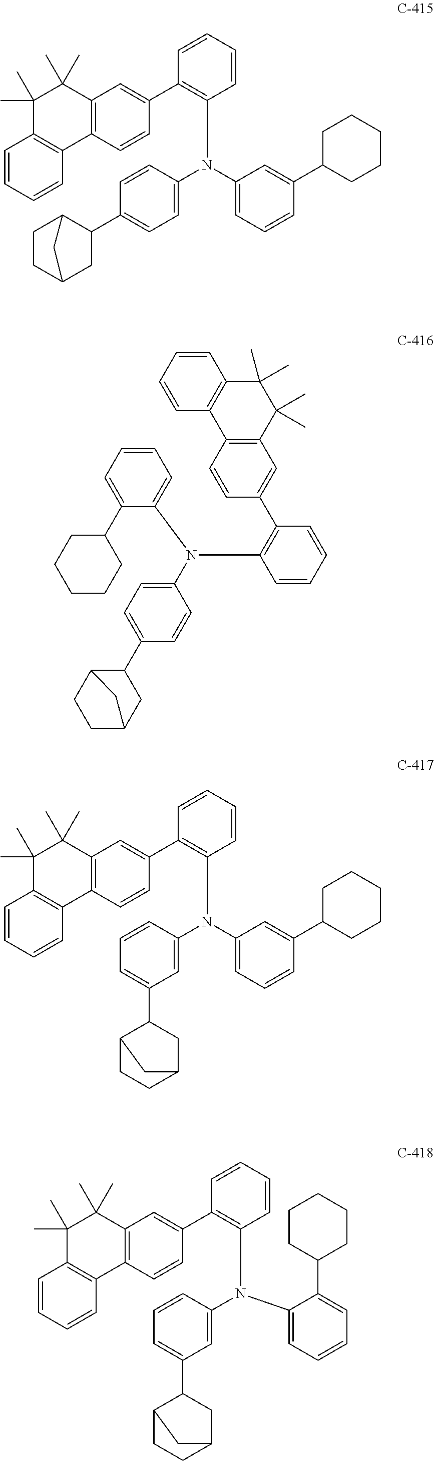 [SMILES: CC1(C)c2ccccc2-c2ccc(-c3ccccc3N(c3ccc(C4CC5CCC4C5)cc3)c3cccc(C4CCCCC4)c3)cc2C1(C)C ; CC1(C)c2ccccc2-c2ccc(-c3ccccc3N(c3ccc(C4CC5CCC4C5)cc3)c3ccccc3C3CCCCC3)cc2C1(C)C ; CC1(C)c2ccccc2-c2ccc(-c3ccccc3N(c3cccc(C4CCCCC4)c3)c3cccc(C4CC5CCC4C5)c3)cc2C1(C)C ; CC1(C)c2ccccc2-c2ccc(-c3ccccc3N(c3cccc(C4CC5CCC4C5)c3)c3ccccc3C3CCCCC3)cc2C1(C)C]

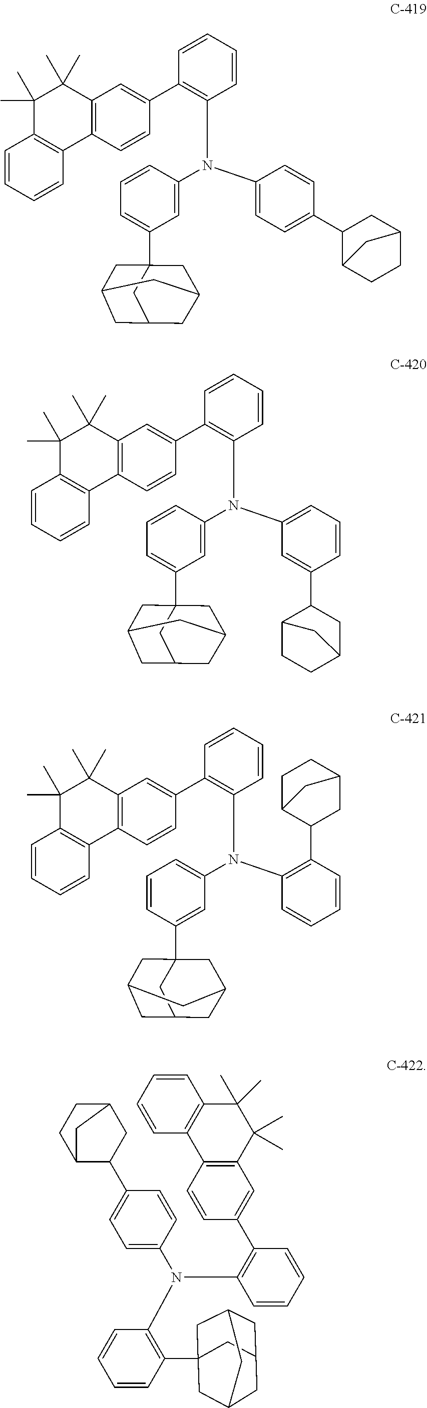 [SMILES: CC1(C)c2ccccc2-c2ccc(-c3ccccc3N(c3ccc(C4CC5CCC4C5)cc3)c3cccc(C45CC6CC(CC(C6)C4)C5)c3)cc2C1(C)C ; CC1(C)c2ccccc2-c2ccc(-c3ccccc3N(c3cccc(C4CC5CCC4C5)c3)c3cccc(C45CC6CC(CC(C6)C4)C5)c3)cc2C1(C)C ; CC1(C)c2ccccc2-c2ccc(-c3ccccc3N(c3cccc(C45CC6CC(CC(C6)C4)C5)c3)c3ccccc3C3CC4CCC3C4)cc2C1(C)C ; CC1(C)c2ccccc2-c2ccc(-c3ccccc3N(c3ccc(C4CC5CCC4C5)cc3)c3ccccc3C34CC5CC(CC(C5)C3)C4)cc2C1(C)C]

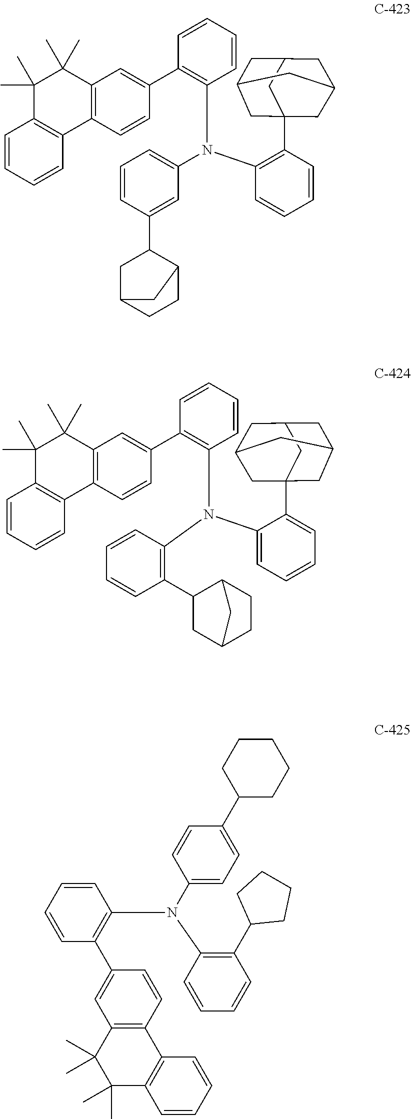 [SMILES: CC1(C)c2ccccc2-c2ccc(-c3ccccc3N(c3cccc(C4CC5CCC4C5)c3)c3ccccc3C34CC5CC(CC(C5)C3)C4)cc2C1(C)C ; CC1(C)c2ccccc2-c2ccc(-c3ccccc3N(c3ccccc3C3CC4CCC3C4)c3ccccc3C34CC5CC(CC(C5)C3)C4)cc2C1(C)C ; CC1(C)c2ccccc2-c2ccc(-c3ccccc3N(c3ccc(C4CCCCC4)cc3)c3ccccc3C3CCCC3)cc2C1(C)C]

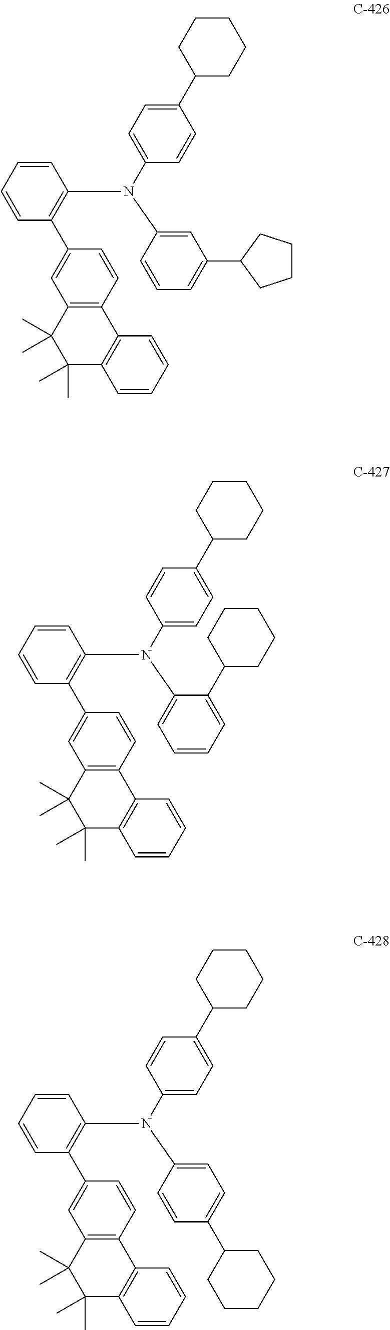 [SMILES: CC1(C)c2ccccc2-c2ccc(-c3ccccc3N(c3ccc(C4CCCCC4)cc3)c3cccc(C4CCCC4)c3)cc2C1(C)C ; CC1(C)c2ccccc2-c2ccc(-c3ccccc3N(c3ccc(C4CCCCC4)cc3)c3ccccc3C3CCCCC3)cc2C1(C)C ; CC1(C)c2ccccc2-c2ccc(-c3ccccc3N(c3ccc(C4CCCCC4)cc3)c3ccc(C4CCCCC4)cc3)cc2C1(C)C]

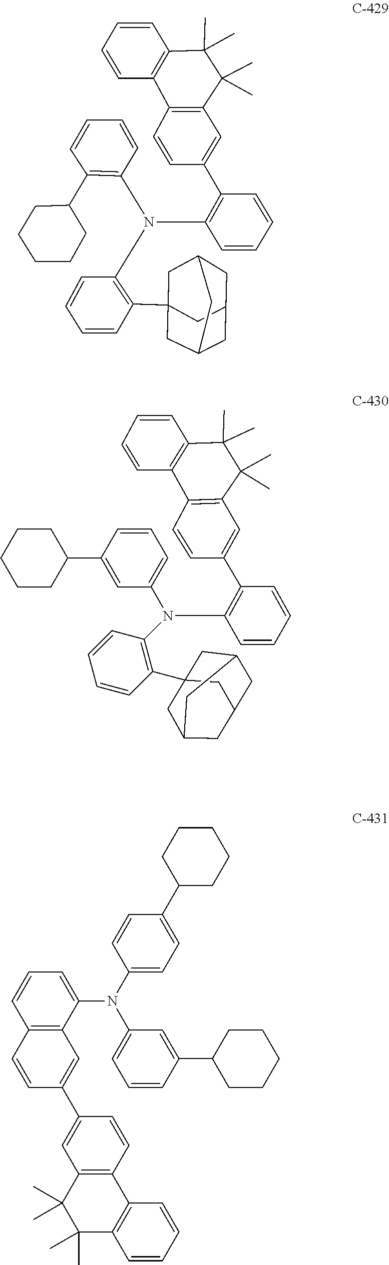 [SMILES: CC1(C)c2ccccc2-c2ccc(-c3ccccc3N(c3ccccc3C3CCCCC3)c3ccccc3C34CC5CC(CC(C5)C3)C4)cc2C1(C)C ; CC1(C)c2ccccc2-c2ccc(-c3ccccc3N(c3cccc(C4CCCCC4)c3)c3ccccc3C34CC5CC(CC(C5)C3)C4)cc2C1(C)C ; CC1(C)c2ccccc2-c2ccc(-c3ccc4cccc(N(c5ccc(C6CCCCC6)cc5)c5cccc(C6CCCCC6)c5)c4c3)cc2C1(C)C]

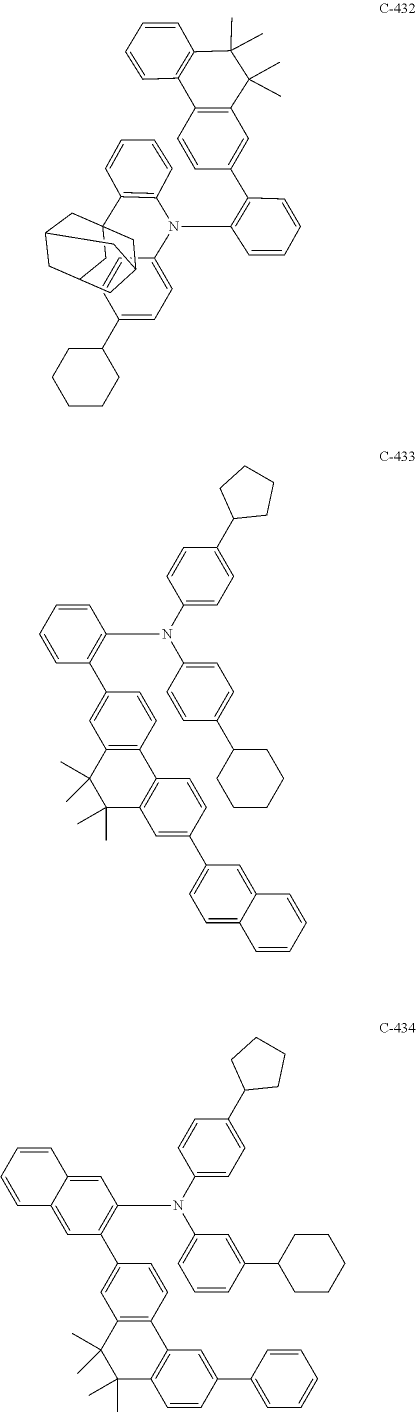 [SMILES: CC1(C)c2ccccc2-c2ccc(-c3ccccc3N(c3ccc(C4CCCCC4)cc3)c3ccccc3C34CC5CC(CC(C5)C3)C4)cc2C1(C)C ; CC1(C)c2cc(-c3ccc4ccccc4c3)ccc2-c2ccc(-c3ccccc3N(c3ccc(C4CCCCC4)cc3)c3ccc(C4CCCC4)cc3)cc2C1(C)C ; CC1(C)c2ccc(-c3ccccc3)cc2-c2ccc(-c3cc4ccccc4cc3N(c3ccc(C4CCCC4)cc3)c3cccc(C4CCCCC4)c3)cc2C1(C)C]

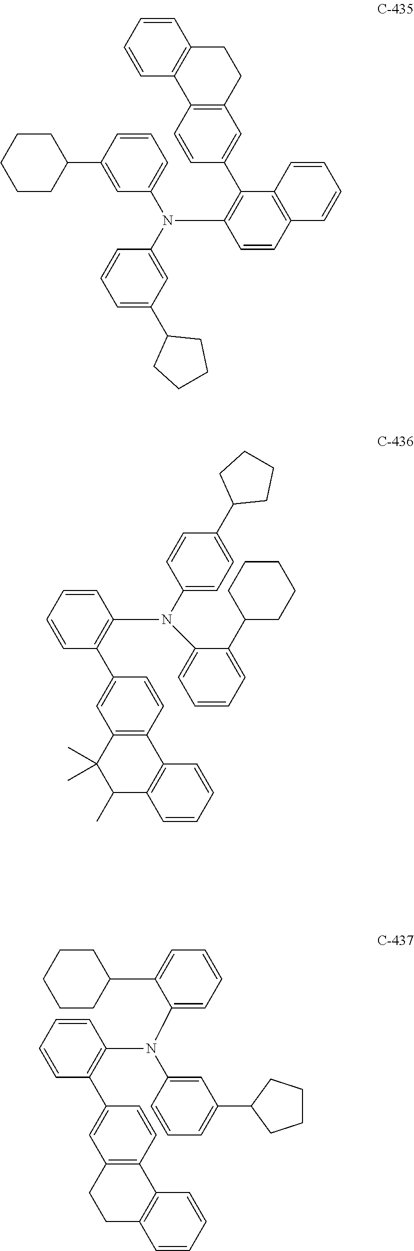 [SMILES: c1cc(C2CCCCC2)cc(N(c2cccc(C3CCCC3)c2)c2ccc3ccccc3c2-c2ccc3c(c2)CCc2ccccc2-3)c1 ; CC1c2ccccc2-c2ccc(-c3ccccc3N(c3ccc(C4CCCC4)cc3)c3ccccc3C3CCCCC3)cc2C1(C)C ; c1cc(C2CCCC2)cc(N(c2ccccc2-c2ccc3c(c2)CCc2ccccc2-3)c2ccccc2C2CCCCC2)c1]

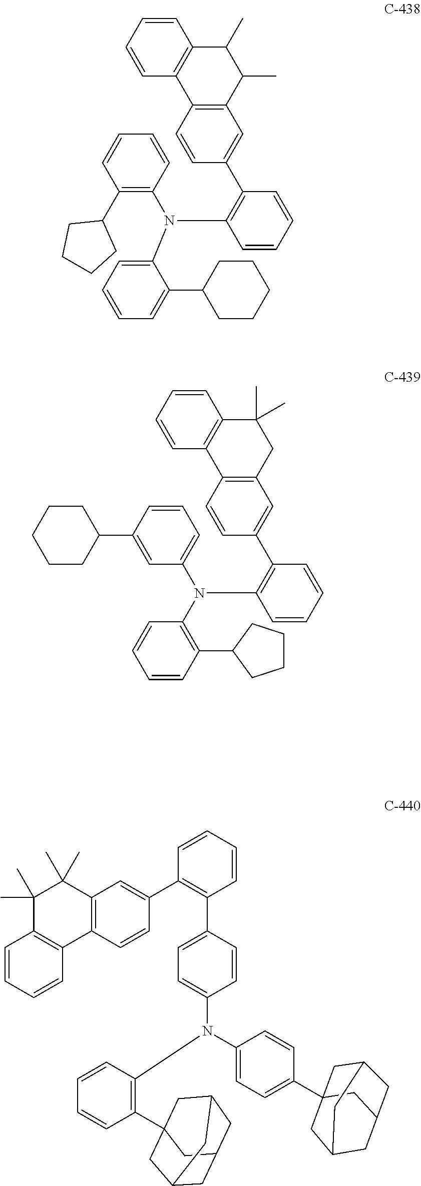 [SMILES: CC1c2ccccc2-c2ccc(-c3ccccc3N(c3ccccc3C3CCCCC3)c3ccccc3C3CCCC3)cc2C1C ; CC1(C)Cc2cc(-c3ccccc3N(c3cccc(C4CCCCC4)c3)c3ccccc3C3CCCC3)ccc2-c2ccccc21 ; CC1(C)c2ccccc2-c2ccc(-c3ccccc3-c3ccc(N(c4ccc(C56CC7CC(CC(C7)C5)C6)cc4)c4ccccc4C45CC6CC(CC(C6)C4)C5)cc3)cc2C1(C)C]

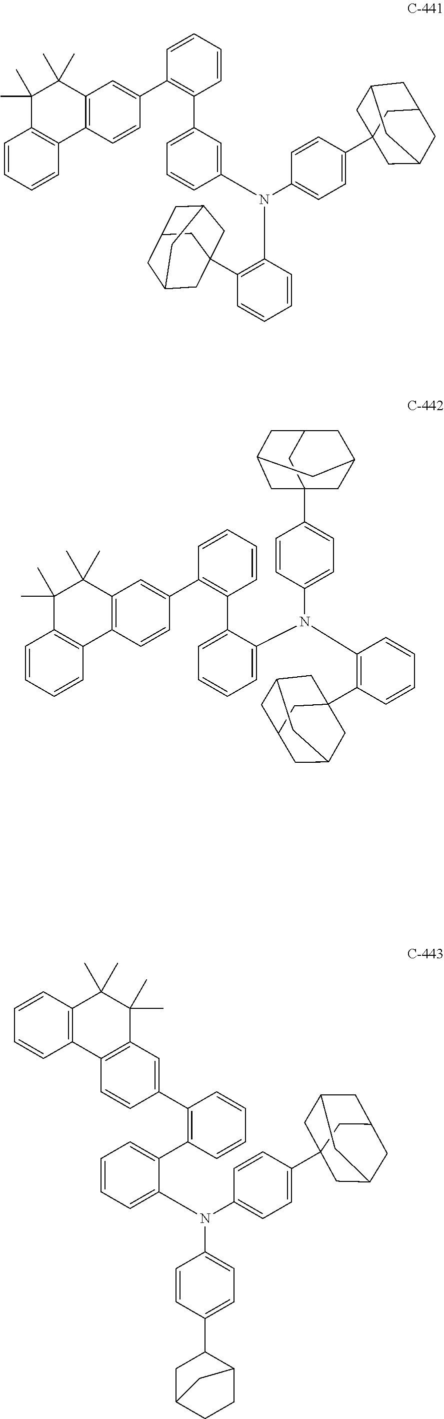 [SMILES: CC1(C)c2ccccc2-c2ccc(-c3ccccc3-c3cccc(N(c4ccc(C56CC7CC(CC(C7)C5)C6)cc4)c4ccccc4C45CC6CC(CC(C6)C4)C5)c3)cc2C1(C)C ; CC1(C)c2ccccc2-c2ccc(-c3ccccc3-c3ccccc3N(c3ccc(C45CC6CC(CC(C6)C4)C5)cc3)c3ccccc3C34CC5CC(CC(C5)C3)C4)cc2C1(C)C ; CC1(C)c2ccccc2-c2ccc(-c3ccccc3-c3ccccc3N(c3ccc(C4CC5CCC4C5)cc3)c3ccc(C45CC6CC(CC(C6)C4)C5)cc3)cc2C1(C)C]

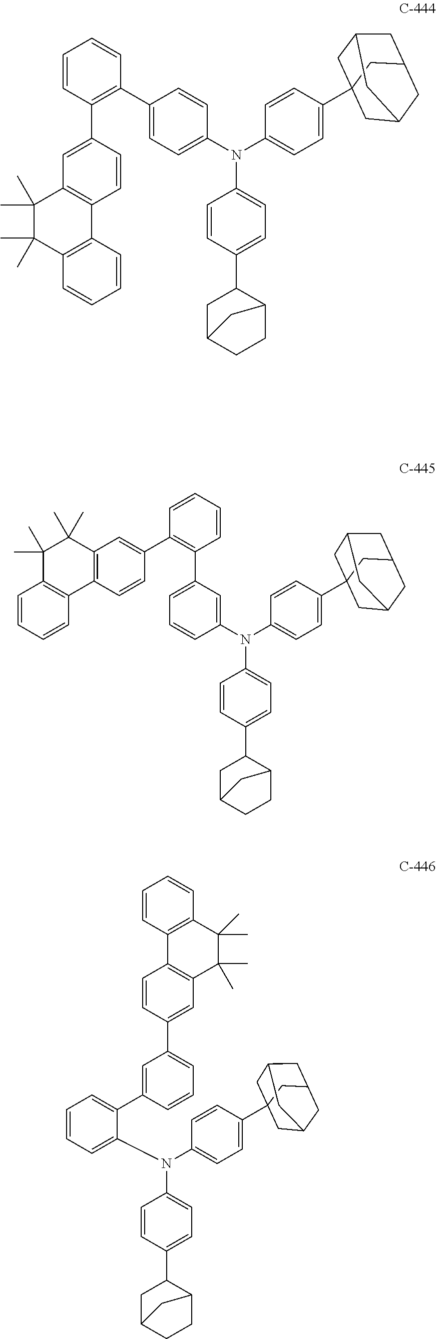 [SMILES: CC1(C)c2ccccc2-c2ccc(-c3ccccc3-c3ccc(N(c4ccc(C5CC6CCC5C6)cc4)c4ccc(C56CC7CC(CC(C7)C5)C6)cc4)cc3)cc2C1(C)C ; CC1(C)c2ccccc2-c2ccc(-c3ccccc3-c3cccc(N(c4ccc(C5CC6CCC5C6)cc4)c4ccc(C56CC7CC(CC(C7)C5)C6)cc4)c3)cc2C1(C)C ; CC1(C)c2ccccc2-c2ccc(-c3cccc(-c4ccccc4N(c4ccc(C5CC6CCC5C6)cc4)c4ccc(C56CC7CC(CC(C7)C5)C6)cc4)c3)cc2C1(C)C]

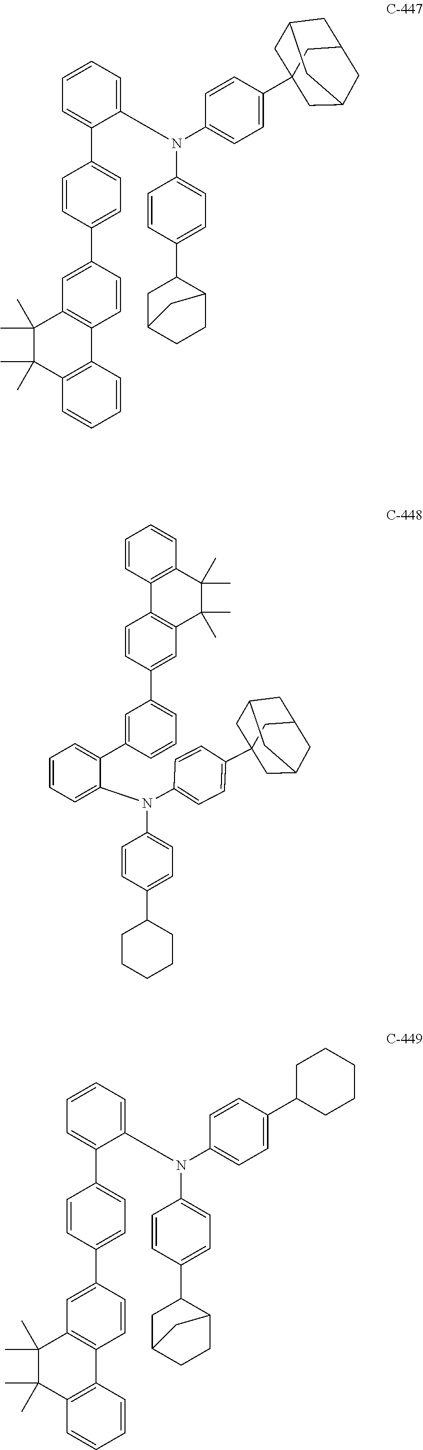 [SMILES: CC1(C)c2ccccc2-c2ccc(-c3ccc(-c4ccccc4N(c4ccc(C5CC6CCC5C6)cc4)c4ccc(C56CC7CC(CC(C7)C5)C6)cc4)cc3)cc2C1(C)C ; CC1(C)c2ccccc2-c2ccc(-c3cccc(-c4ccccc4N(c4ccc(C5CCCCC5)cc4)c4ccc(C56CC7CC(CC(C7)C5)C6)cc4)c3)cc2C1(C)C ; CC1(C)c2ccccc2-c2ccc(-c3ccc(-c4ccccc4N(c4ccc(C5CCCCC5)cc4)c4ccc(C5CC6CCC5C6)cc4)cc3)cc2C1(C)C]

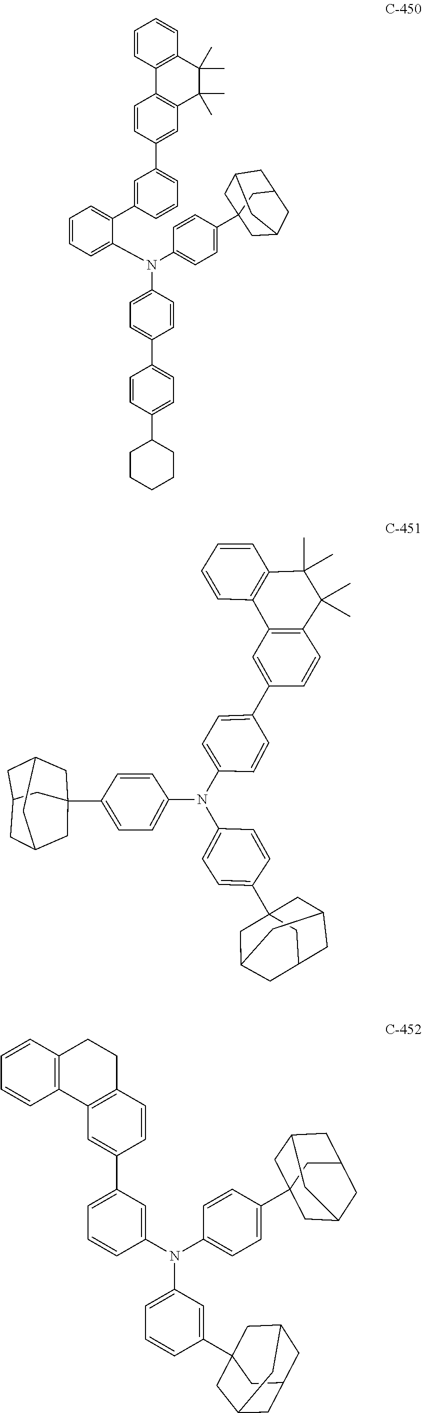 [SMILES: CC1(C)c2ccccc2-c2ccc(-c3cccc(-c4ccccc4N(c4ccc(-c5ccc(C6CCCCC6)cc5)cc4)c4ccc(C56CC7CC(CC(C7)C5)C6)cc4)c3)cc2C1(C)C ; CC1(C)c2ccccc2-c2cc(-c3ccc(N(c4ccc(C56CC7CC(CC(C7)C5)C6)cc4)c4ccc(C56CC7CC(CC(C7)C5)C6)cc4)cc3)ccc2C1(C)C ; c1cc(-c2ccc3c(c2)-c2ccccc2CC3)cc(N(c2ccc(C34CC5CC(CC(C5)C3)C4)cc2)c2cccc(C34CC5CC(CC(C5)C3)C4)c2)c1]

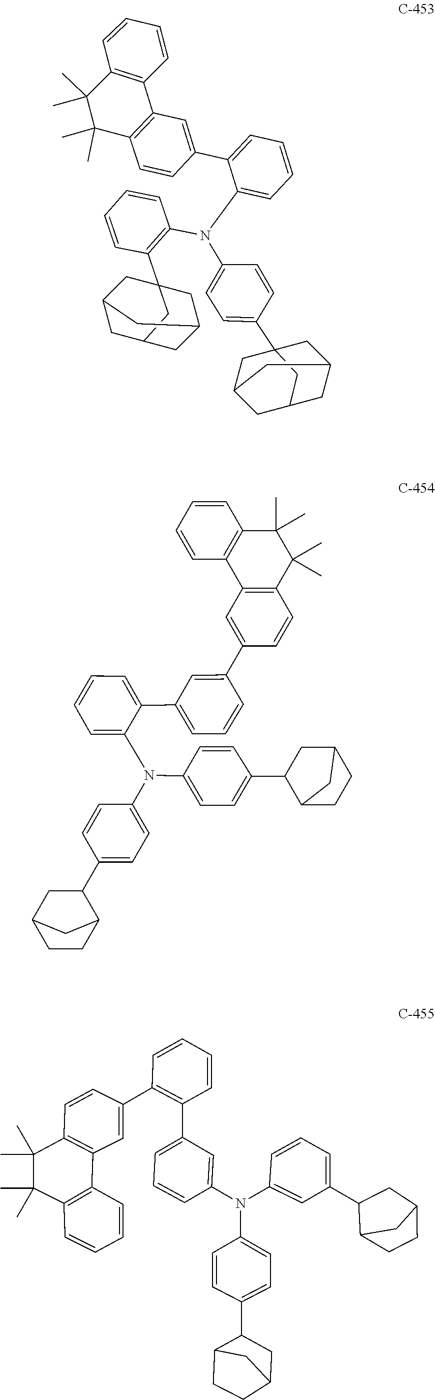 [SMILES: CC1(C)c2ccccc2-c2cc(-c3ccccc3N(c3ccc(C45CC6CC(CC(C6)C4)C5)cc3)c3ccccc3C34CC5CC(CC(C5)C3)C4)ccc2C1(C)C ; CC1(C)c2ccccc2-c2cc(-c3cccc(-c4ccccc4N(c4ccc(C5CC6CCC5C6)cc4)c4ccc(C5CC6CCC5C6)cc4)c3)ccc2C1(C)C ; CC1(C)c2ccccc2-c2cc(-c3ccccc3-c3cccc(N(c4ccc(C5CC6CCC5C6)cc4)c4cccc(C5CC6CCC5C6)c4)c3)ccc2C1(C)C]

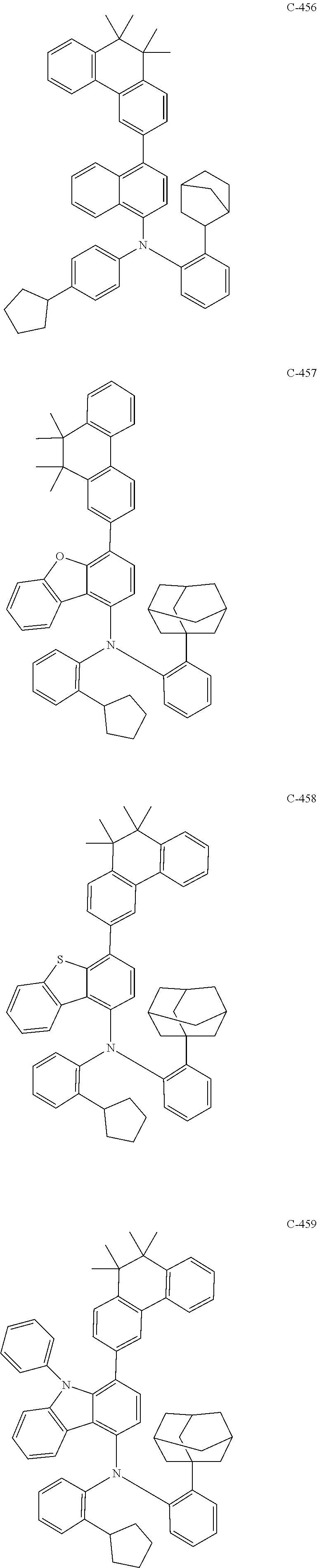 [SMILES: CC1(C)c2ccccc2-c2cc(-c3ccc(N(c4ccc(C5CCCC5)cc4)c4ccccc4C4CC5CCC4C5)c4ccccc34)ccc2C1(C)C ; CC1(C)c2ccccc2-c2ccc(-c3ccc(N(c4ccccc4C4CCCC4)c4ccccc4C45CC6CC(CC(C6)C4)C5)c4c3oc3ccccc34)cc2C1(C)C ; CC1(C)c2ccccc2-c2cc(-c3ccc(N(c4ccccc4C4CCCC4)c4ccccc4C45CC6CC(CC(C6)C4)C5)c4c3sc3ccccc34)ccc2C1(C)C ; CC1(C)c2ccccc2-c2cc(-c3ccc(N(c4ccccc4C4CCCC4)c4ccccc4C45CC6CC(CC(C6)C4)C5)c4c5ccccc5n(-c5ccccc5)c34)ccc2C1(C)C]

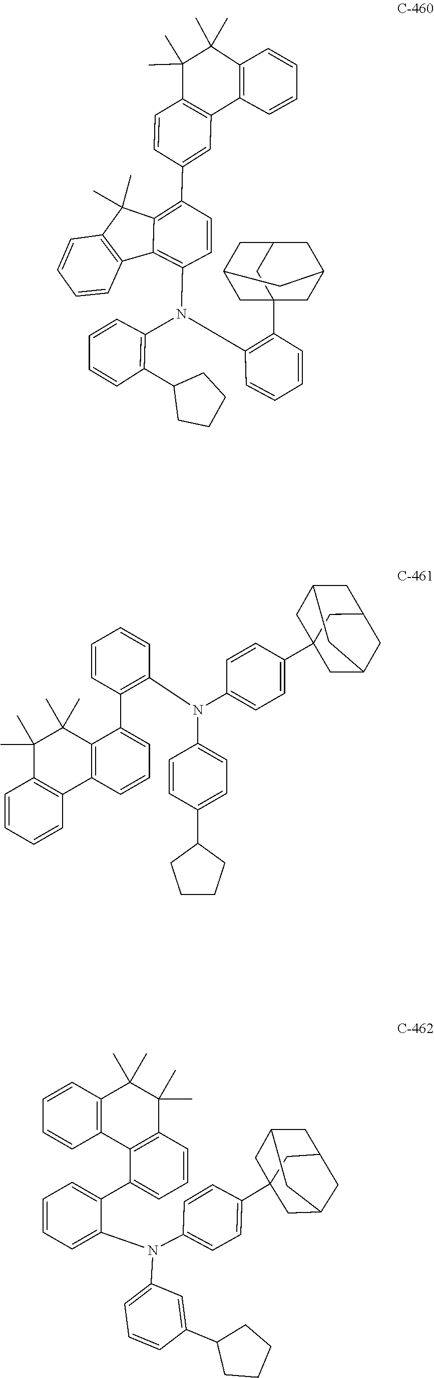 [SMILES: CC1(C)c2ccccc2-c2c(N(c3ccccc3C3CCCC3)c3ccccc3C34CC5CC(CC(C5)C3)C4)ccc(-c3ccc4c(c3)-c3ccccc3C(C)(C)C4(C)C)c21 ; CC1(C)c2ccccc2-c2cccc(-c3ccccc3N(c3ccc(C4CCCC4)cc3)c3ccc(C45CC6CC(CC(C6)C4)C5)cc3)c2C1(C)C ; CC1(C)c2ccccc2-c2c(-c3ccccc3N(c3ccc(C45CC6CC(CC(C6)C4)C5)cc3)c3cccc(C4CCCC4)c3)cccc2C1(C)C]

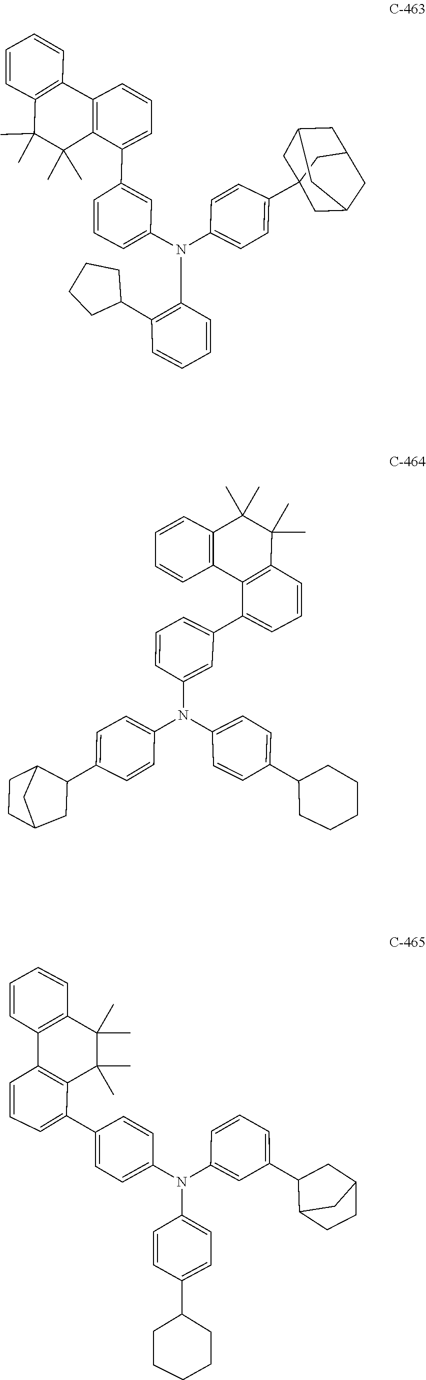 [SMILES: CC1(C)c2ccccc2-c2cccc(-c3cccc(N(c4ccc(C56CC7CC(CC(C7)C5)C6)cc4)c4ccccc4C4CCCC4)c3)c2C1(C)C ; CC1(C)c2ccccc2-c2c(-c3cccc(N(c4ccc(C5CCCCC5)cc4)c4ccc(C5CC6CCC5C6)cc4)c3)cccc2C1(C)C ; CC1(C)c2ccccc2-c2cccc(-c3ccc(N(c4ccc(C5CCCCC5)cc4)c4cccc(C5CC6CCC5C6)c4)cc3)c2C1(C)C]

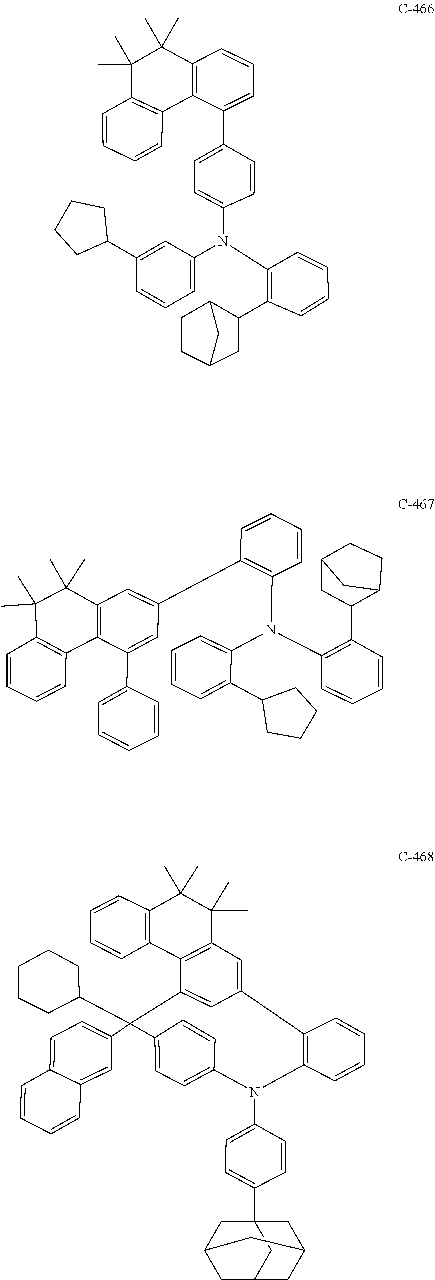 [SMILES: CC1(C)c2ccccc2-c2c(-c3ccc(N(c4cccc(C5CCCC5)c4)c4ccccc4C4CC5CCC4C5)cc3)cccc2C1(C)C ; CC1(C)c2ccccc2-c2c(-c3ccccc3)cc(-c3ccccc3N(c3ccccc3C3CCCC3)c3ccccc3C3CC4CCC3C4)cc2C1(C)C ; CC1(C)c2ccccc2-c2c3cc(cc2C1(C)C)-c1ccccc1N(c1ccc(C24CC5CC(CC(C5)C2)C4)cc1)c1ccc(cc1)C3(c1ccc2ccccc2c1)C1CCCCC1]

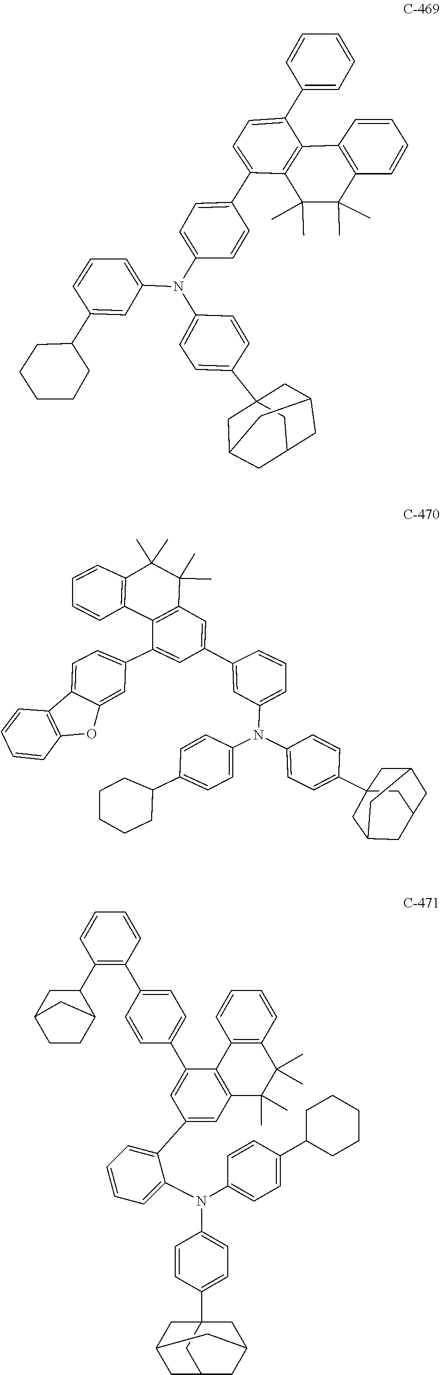 [SMILES: CC1(C)c2ccccc2-c2c(-c3ccccc3)ccc(-c3ccc(N(c4ccc(C56CC7CC(CC(C7)C5)C6)cc4)c4cccc(C5CCCCC5)c4)cc3)c2C1(C)C ; CC1(C)c2ccccc2-c2c(-c3ccc4c(c3)oc3ccccc34)cc(-c3cccc(N(c4ccc(C5CCCCC5)cc4)c4ccc(C56CC7CC(CC(C7)C5)C6)cc4)c3)cc2C1(C)C ; CC1(C)c2ccccc2-c2c(-c3ccc(-c4ccccc4C4CC5CCC4C5)cc3)cc(-c3ccccc3N(c3ccc(C4CCCCC4)cc3)c3ccc(C45CC6CC(CC(C6)C4)C5)cc3)cc2C1(C)C]

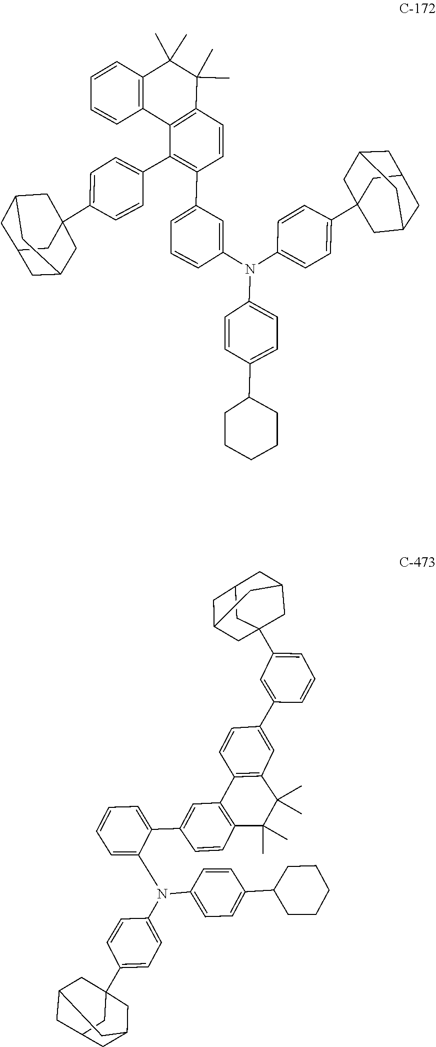 [SMILES: CC1(C)c2ccccc2-c2c(ccc(-c3cccc(N(c4ccc(C5CCCCC5)cc4)c4ccc(C56CC7CC(CC(C7)C5)C6)cc4)c3)c2-c2ccc(C34CC5CC(CC(C5)C3)C4)cc2)C1(C)C ; CC1(C)c2ccc(-c3ccccc3N(c3ccc(C4CCCCC4)cc3)c3ccc(C45CC6CC(CC(C6)C4)C5)cc3)cc2-c2ccc(-c3cccc(C45CC6CC(CC(C6)C4)C5)c3)cc2C1(C)C]

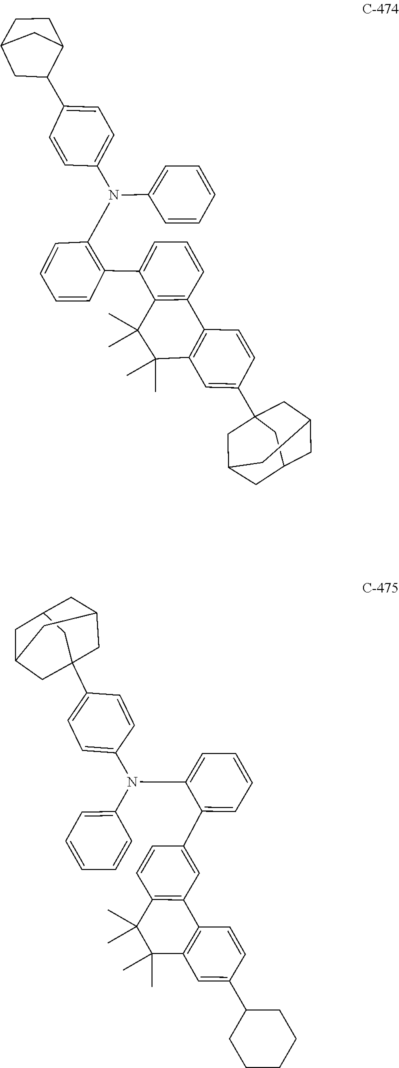 [SMILES: CC1(C)c2cc(C34CC5CC(CC(C5)C3)C4)ccc2-c2cccc(-c3ccccc3N(c3ccccc3)c3ccc(C4CC5CCC4C5)cc3)c2C1(C)C ; CC1(C)c2ccc(-c3ccccc3N(c3ccccc3)c3ccc(C45CC6CC(CC(C6)C4)C5)cc3)cc2-c2ccc(C3CCCCC3)cc2C1(C)C]

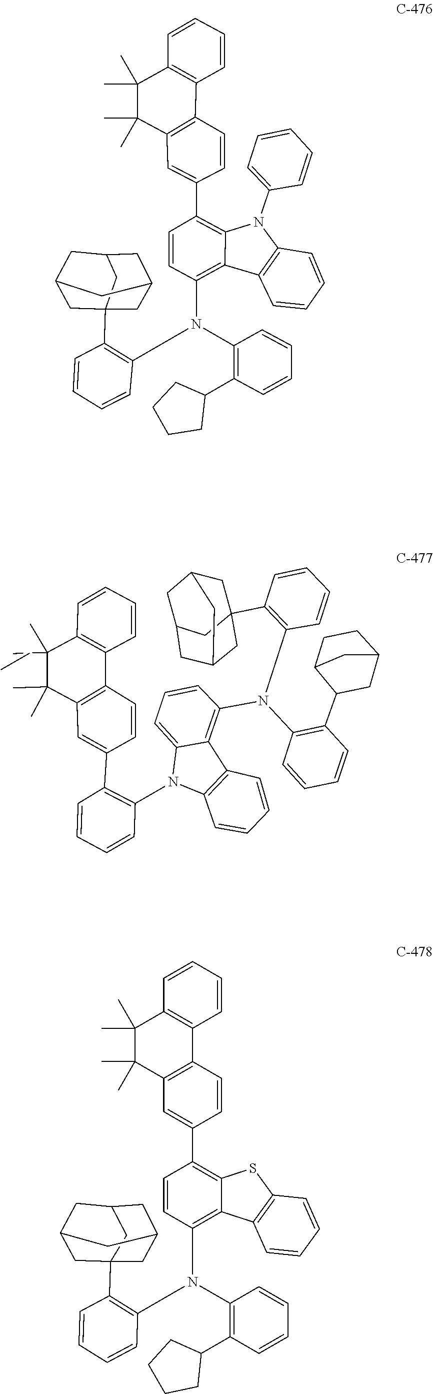

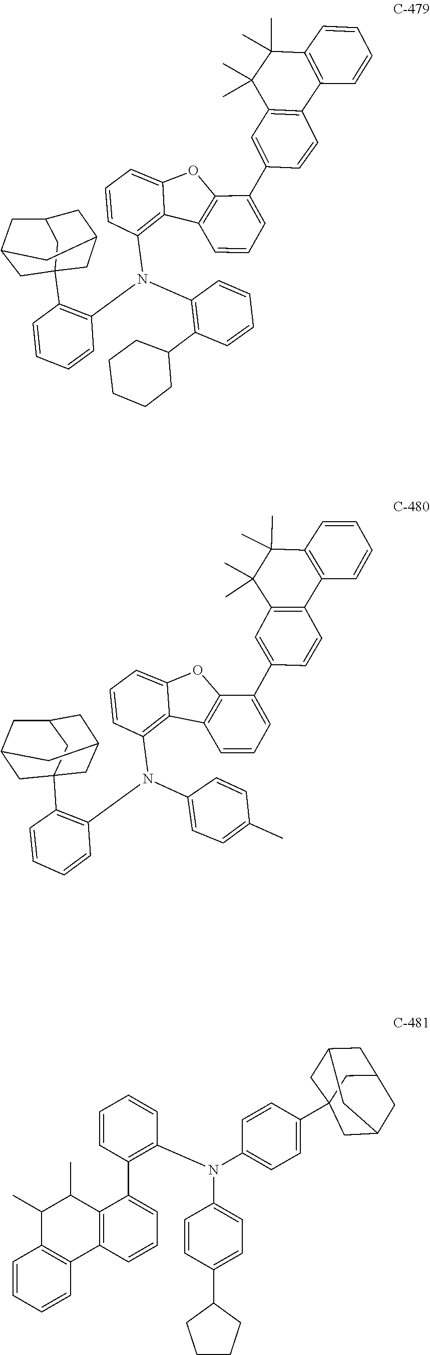 [SMILES: CC1(C)c2ccccc2-c2ccc(-c3cccc4c3oc3cccc(N(c5ccccc5C5CCCCC5)c5ccccc5C56CC7CC(CC(C7)C5)C6)c34)cc2C1(C)C ; Cc1ccc(N(c2ccccc2C23CC4CC(CC(C4)C2)C3)c2cccc3oc4c(-c5ccc6c(c5)C(C)(C)C(C)(C)c5ccccc5-6)cccc4c23)cc1 ; CC1c2ccccc2-c2cccc(-c3ccccc3N(c3ccc(C4CCCC4)cc3)c3ccc(C45CC6CC(CC(C6)C4)C5)cc3)c2C1C]

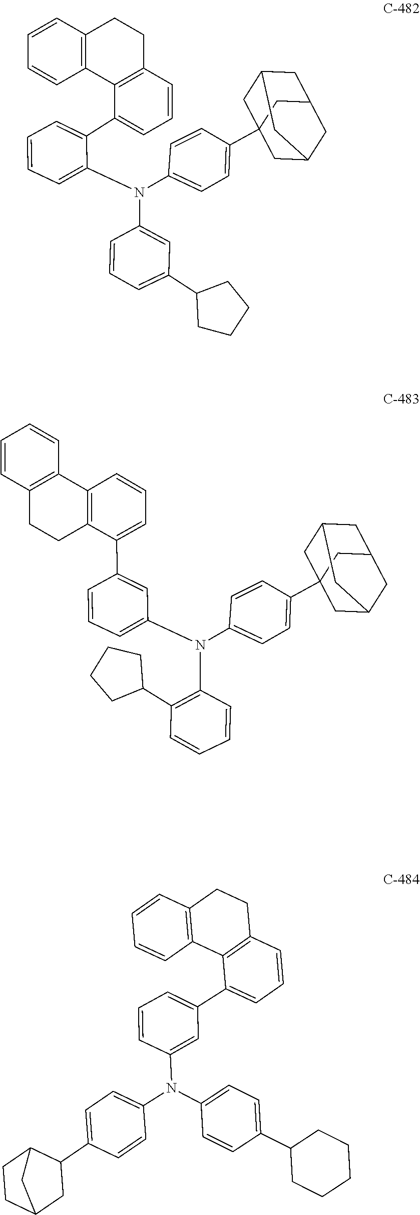 [SMILES: c1cc(C2CCCC2)cc(N(c2ccc(C34CC5CC(CC(C5)C3)C4)cc2)c2ccccc2-c2cccc3c2-c2ccccc2CC3)c1 ; c1cc(-c2cccc3c2CCc2ccccc2-3)cc(N(c2ccc(C34CC5CC(CC(C5)C3)C4)cc2)c2ccccc2C2CCCC2)c1 ; c1cc(-c2cccc3c2-c2ccccc2CC3)cc(N(c2ccc(C3CCCCC3)cc2)c2ccc(C3CC4CCC3C4)cc2)c1]

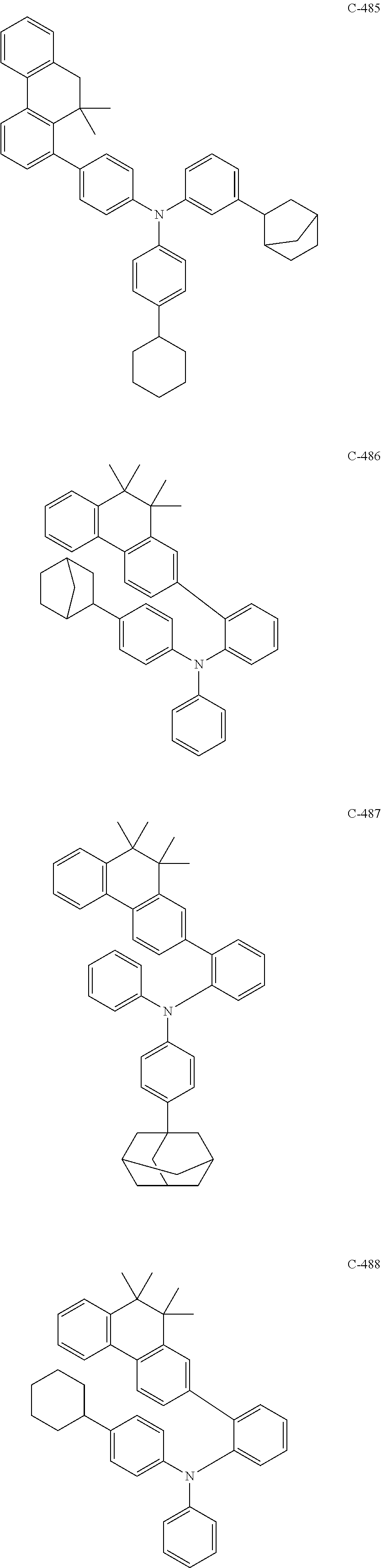 [SMILES: CC1(C)Cc2ccccc2-c2cccc(-c3ccc(N(c4ccc(C5CCCCC5)cc4)c4cccc(C5CC6CCC5C6)c4)cc3)c21 ; CC1(C)c2ccccc2-c2ccc(-c3ccccc3N(c3ccccc3)c3ccc(C4CC5CCC4C5)cc3)cc2C1(C)C ; CC1(C)c2ccccc2-c2ccc(-c3ccccc3N(c3ccccc3)c3ccc(C45CC6CC(CC(C6)C4)C5)cc3)cc2C1(C)C ; CC1(C)c2ccccc2-c2ccc(-c3ccccc3N(c3ccccc3)c3ccc(C4CCCCC4)cc3)cc2C1(C)C]

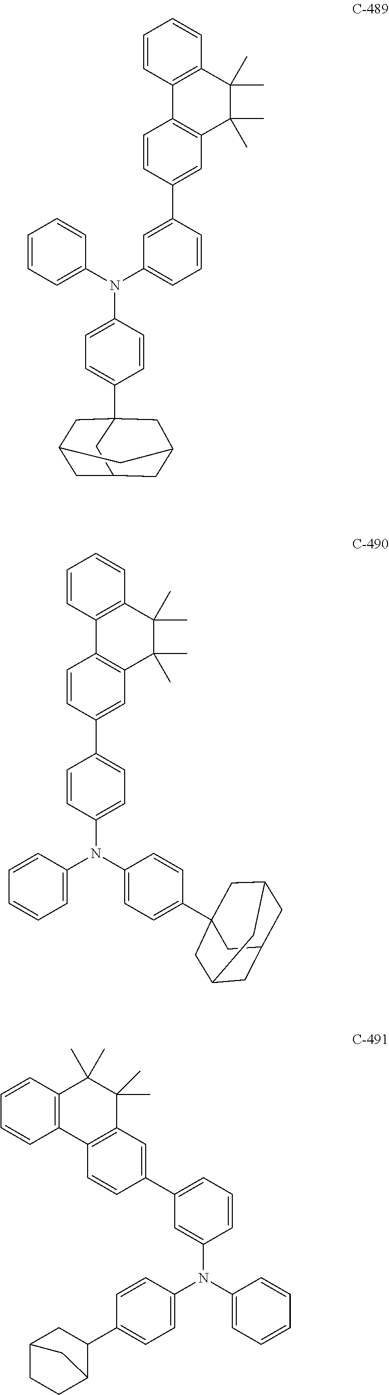 [SMILES: CC1(C)c2ccccc2-c2ccc(-c3cccc(N(c4ccccc4)c4ccc(C56CC7CC(CC(C7)C5)C6)cc4)c3)cc2C1(C)C ; CC1(C)c2ccccc2-c2ccc(-c3ccc(N(c4ccccc4)c4ccc(C56CC7CC(CC(C7)C5)C6)cc4)cc3)cc2C1(C)C ; CC1(C)c2ccccc2-c2ccc(-c3cccc(N(c4ccccc4)c4ccc(C5CC6CCC5C6)cc4)c3)cc2C1(C)C]

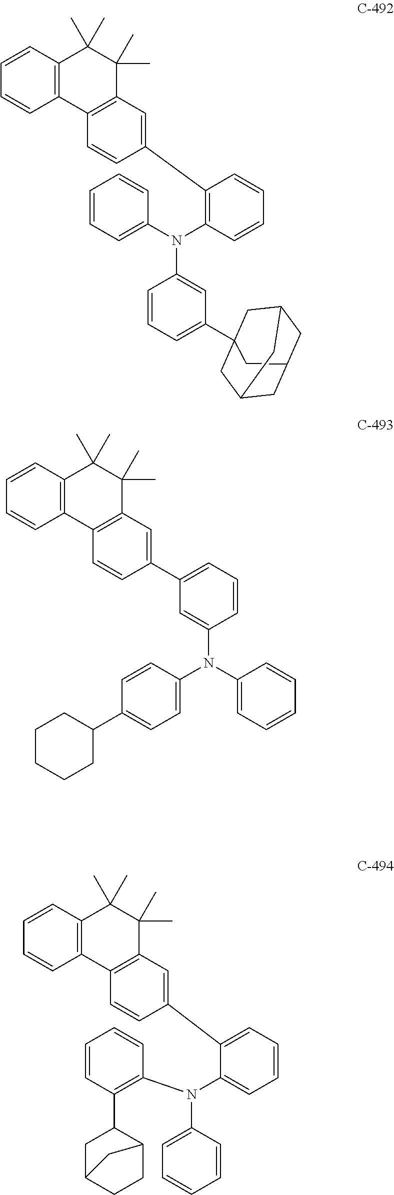 [SMILES: CC1(C)c2ccccc2-c2ccc(-c3ccccc3N(c3ccccc3)c3cccc(C45CC6CC(CC(C6)C4)C5)c3)cc2C1(C)C ; CC1(C)c2ccccc2-c2ccc(-c3cccc(N(c4ccccc4)c4ccc(C5CCCCC5)cc4)c3)cc2C1(C)C ; CC1(C)c2ccccc2-c2ccc(-c3ccccc3N(c3ccccc3)c3ccccc3C3CC4CCC3C4)cc2C1(C)C]

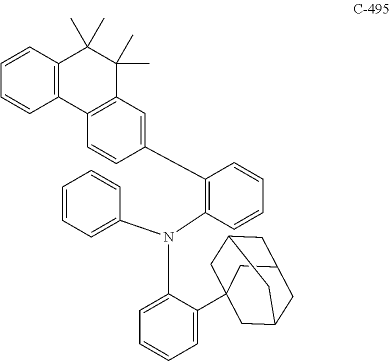 [SMILES: CC1(C)c2ccccc2-c2ccc(-c3ccccc3N(c3ccccc3)c3ccccc3C34CC5CC(CC(C5)C3)C4)cc2C1(C)C]

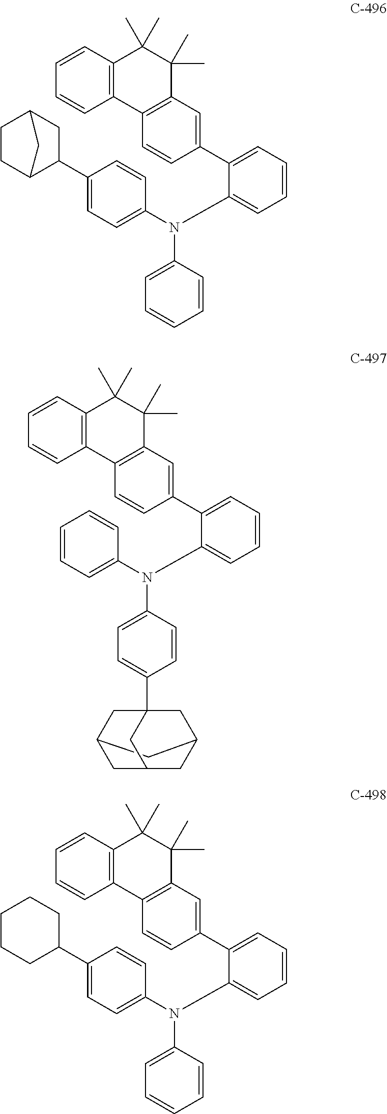

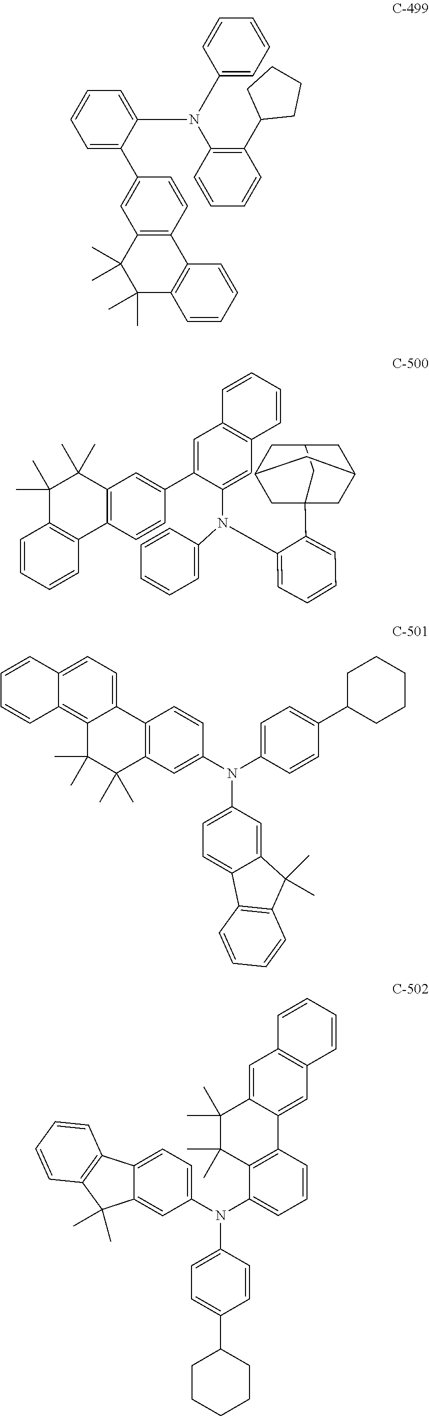 [SMILES: CC1(C)c2ccccc2-c2ccc(-c3ccccc3N(c3ccccc3)c3ccccc3C3CCCC3)cc2C1(C)C ; CC1(C)c2ccccc2-c2ccc(-c3cc4ccccc4cc3N(c3ccccc3)c3ccccc3C34CC5CC(CC(C5)C3)C4)cc2C1(C)C ; CC1(C)c2ccccc2-c2ccc(N(c3ccc(C4CCCCC4)cc3)c3ccc4c(c3)C(C)(C)C(C)(C)c3c-4ccc4ccccc34)cc21 ; CC1(C)c2ccccc2-c2ccc(N(c3ccc(C4CCCCC4)cc3)c3cccc4c3C(C)(C)C(C)(C)c3cc5ccccc5cc3-4)cc21]

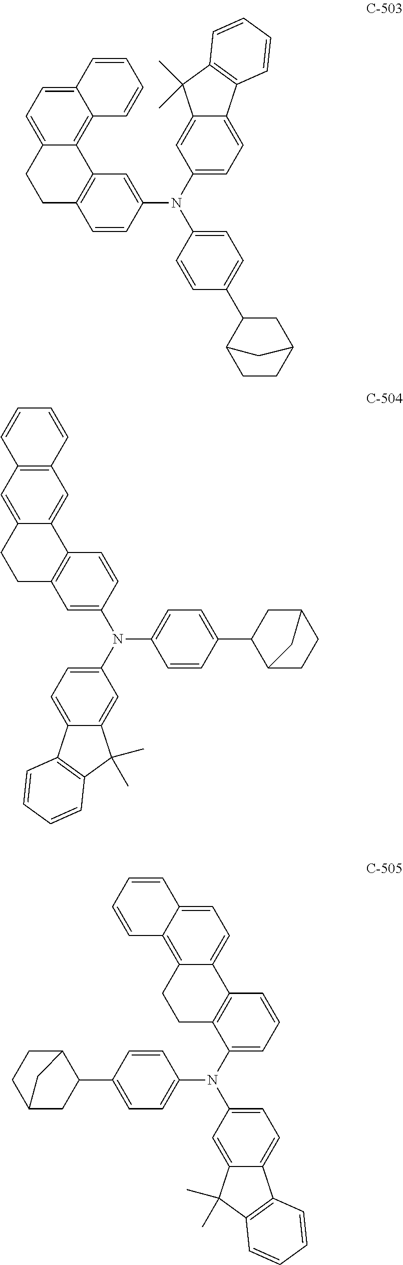 [SMILES: CC1(C)c2ccccc2-c2ccc(N(c3ccc(C4CC5CCC4C5)cc3)c3ccc4c(c3)-c3c(ccc5ccccc35)CC4)cc21 ; CC1(C)c2ccccc2-c2ccc(N(c3ccc(C4CC5CCC4C5)cc3)c3ccc4c(c3)CCc3cc5ccccc5cc3-4)cc21 ; CC1(C)c2ccccc2-c2ccc(N(c3ccc(C4CC5CCC4C5)cc3)c3cccc4c3CCc3c-4ccc4ccccc34)cc21]

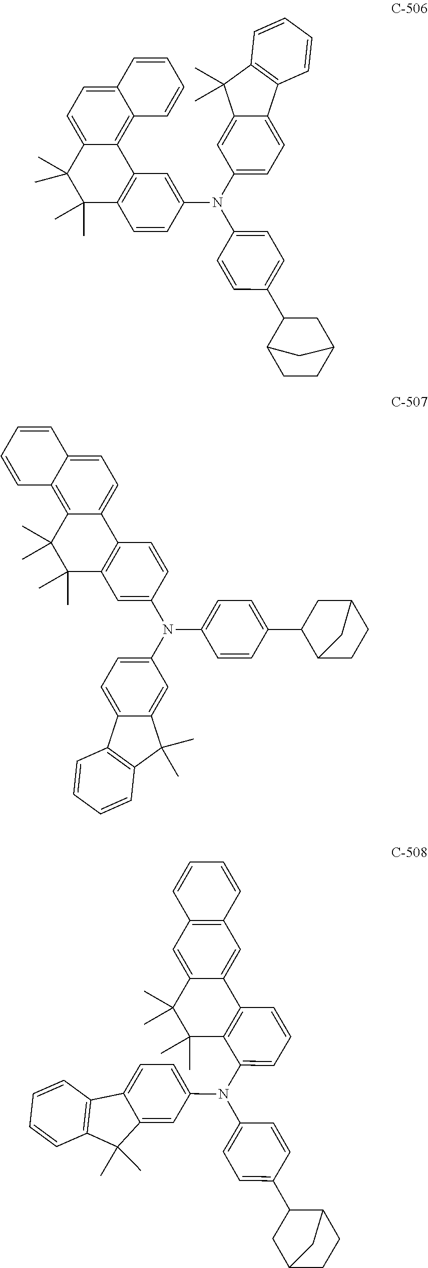 [SMILES: CC1(C)c2ccccc2-c2ccc(N(c3ccc(C4CC5CCC4C5)cc3)c3ccc4c(c3)-c3c(ccc5ccccc35)C(C)(C)C4(C)C)cc21 ; CC1(C)c2ccccc2-c2ccc(N(c3ccc(C4CC5CCC4C5)cc3)c3ccc4c(c3)C(C)(C)C(C)(C)c3c-4ccc4ccccc34)cc21 ; CC1(C)c2ccccc2-c2ccc(N(c3ccc(C4CC5CCC4C5)cc3)c3cccc4c3C(C)(C)C(C)(C)c3cc5ccccc5cc3-4)cc21]

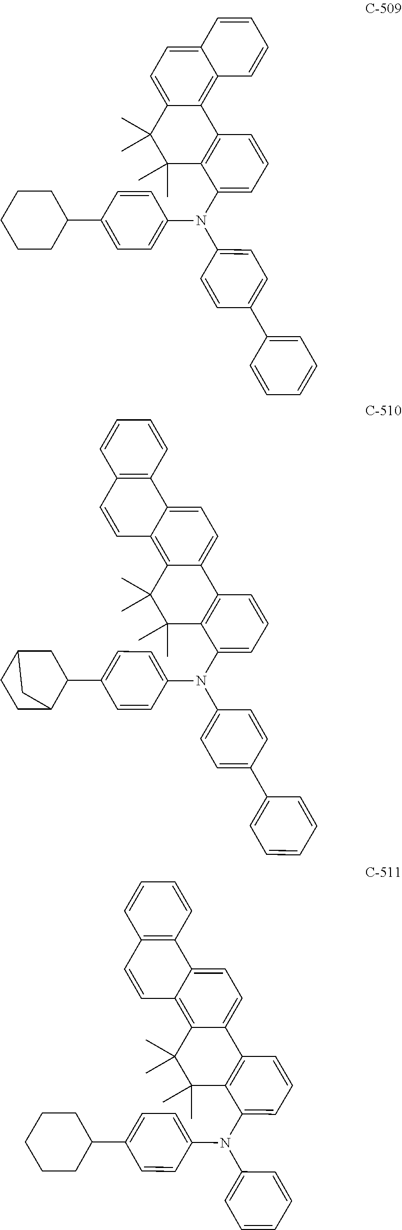 [SMILES: CC1(C)c2ccc3ccccc3c2-c2cccc(N(c3ccc(-c4ccccc4)cc3)c3ccc(C4CCCCC4)cc3)c2C1(C)C ; CC1(C)c2c(cccc2N(c2ccc(-c3ccccc3)cc2)c2ccc(C3CC4CCC3C4)cc2)-c2ccc3c(ccc4ccccc43)c2C1(C)C ; CC1(C)c2c(cccc2N(c2ccccc2)c2ccc(C3CCCCC3)cc2)-c2ccc3c(ccc4ccccc43)c2C1(C)C]

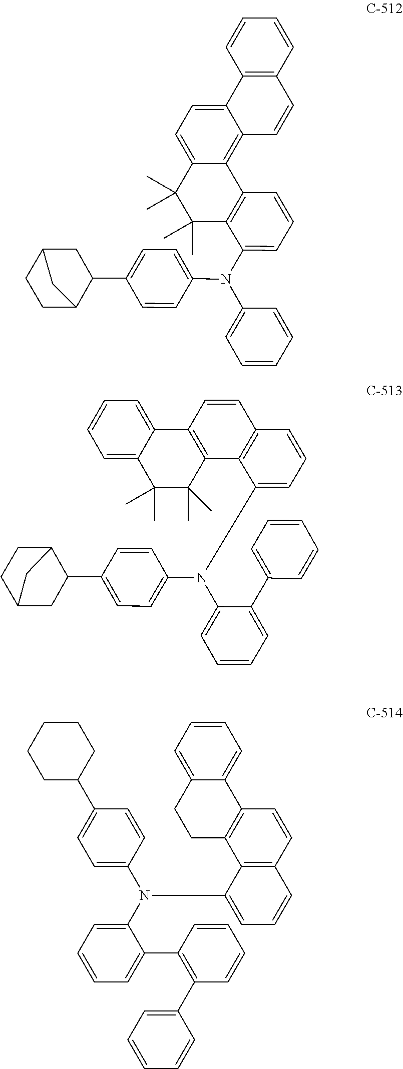

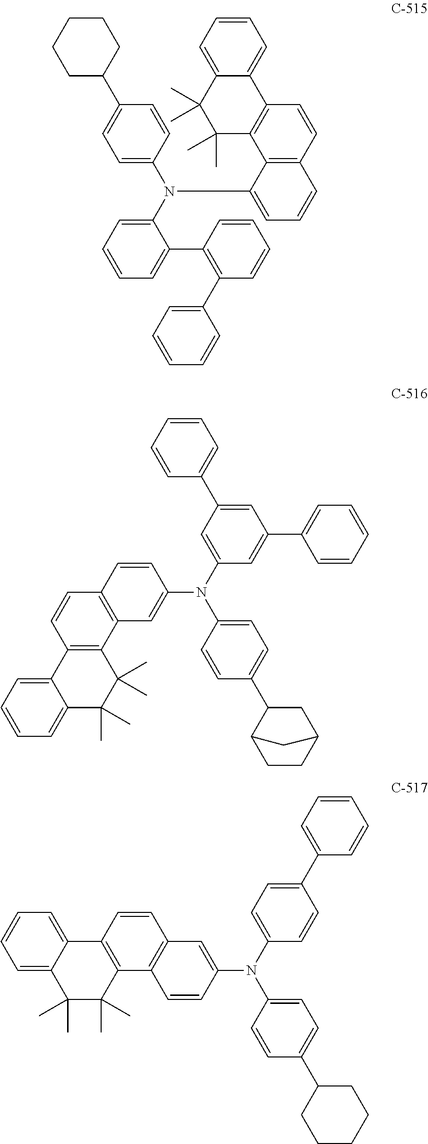

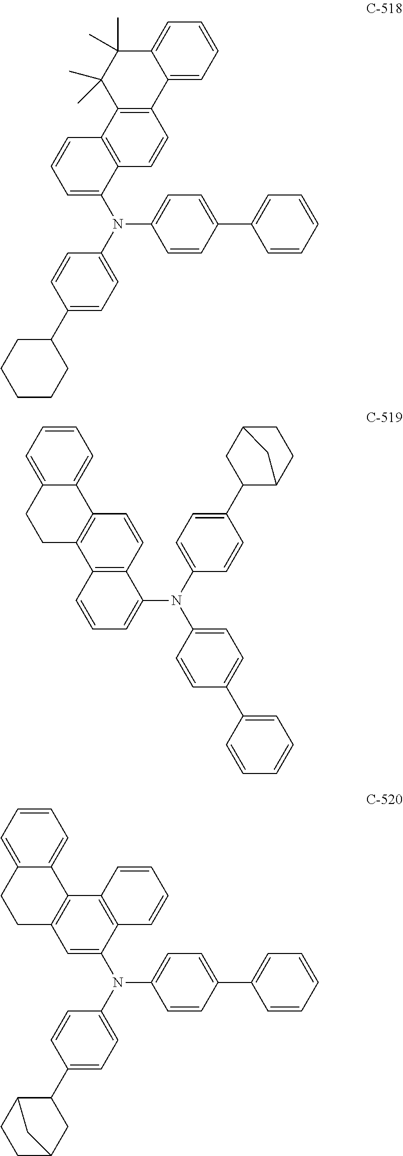

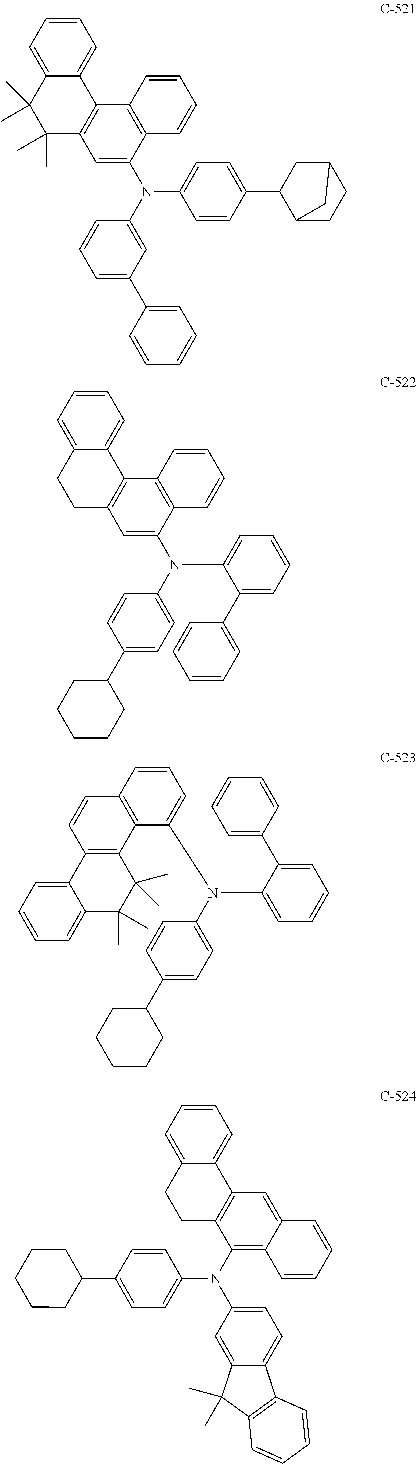

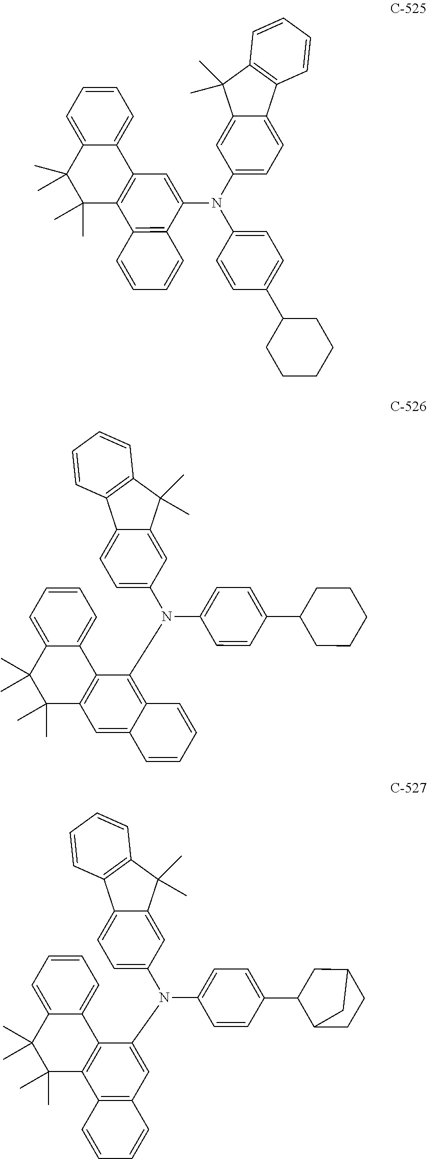 [SMILES: CC1(C)c2ccccc2-c2ccc(N(c3ccc(C4CCCCC4)cc3)c3cc4c(c5ccccc35)C(C)(C)C(C)(C)c3ccccc3-4)cc21 ; CC1(C)c2ccccc2-c2ccc(N(c3ccc(C4CCCCC4)cc3)c3c4c(cc5ccccc35)C(C)(C)C(C)(C)c3ccccc3-4)cc21 ; CC1(C)c2ccccc2-c2ccc(N(c3ccc(C4CC5CCC4C5)cc3)c3cc4ccccc4c4c3-c3ccccc3C(C)(C)C4(C)C)cc21]

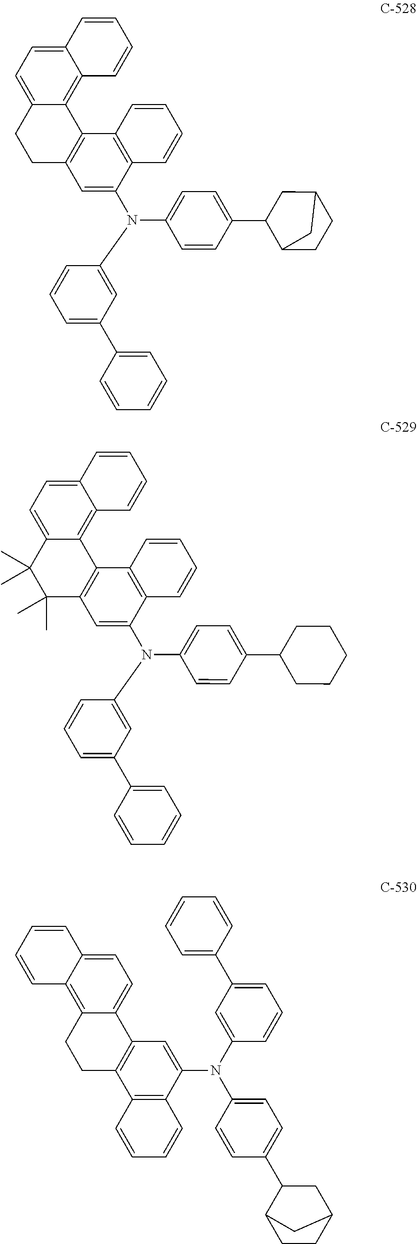

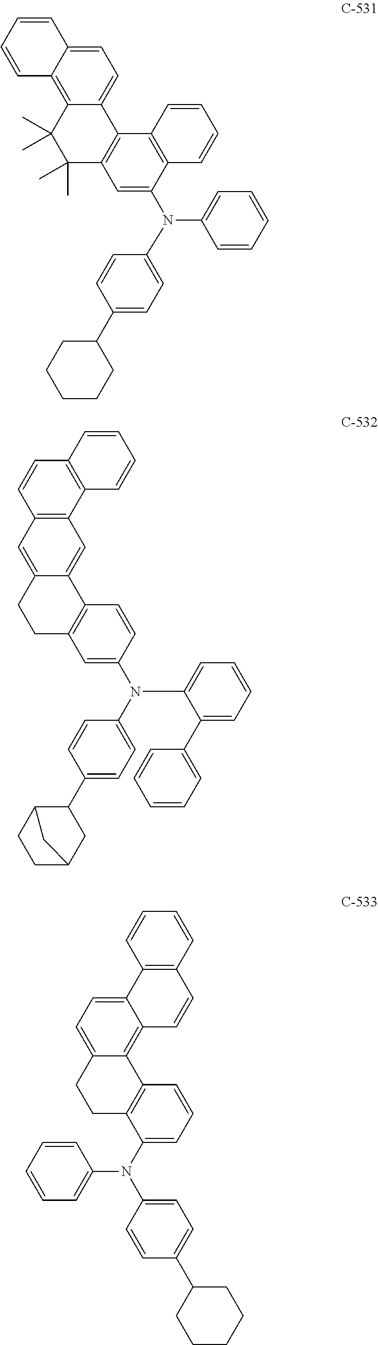

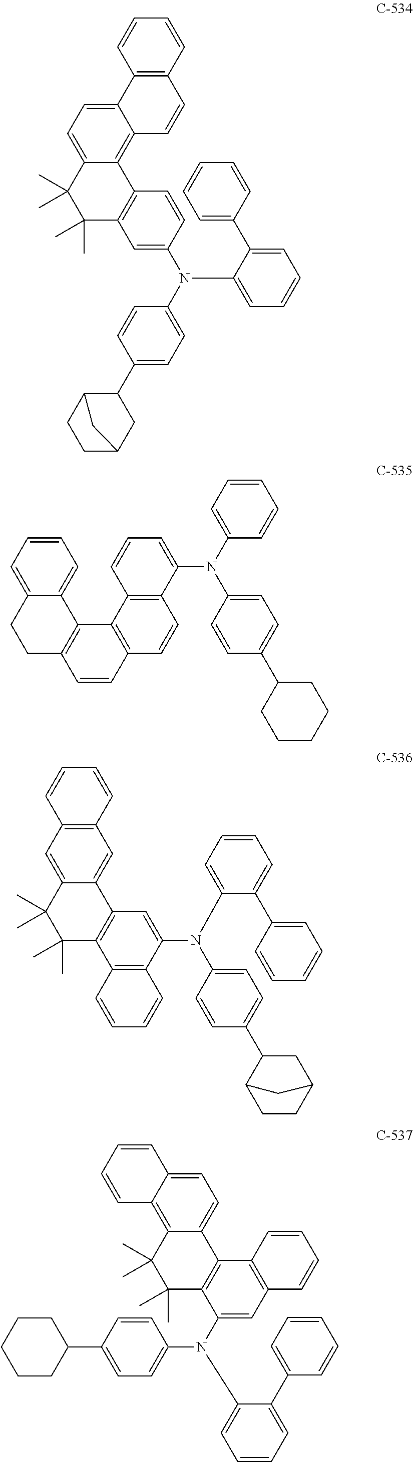 [SMILES: CC1(C)c2cc(N(c3ccc(C4CC5CCC4C5)cc3)c3ccccc3-c3ccccc3)ccc2-c2c(ccc3c2ccc2ccccc23)C1(C)C ; c1ccc(N(c2ccc(C3CCCCC3)cc2)c2cccc3c2ccc2ccc4c(c23)-c2ccccc2CC4)cc1 ; CC1(C)c2cc3ccccc3cc2-c2cc(N(c3ccc(C4CC5CCC4C5)cc3)c3ccccc3-c3ccccc3)c3ccccc3c2C1(C)C ; CC1(C)c2c(N(c3ccc(C4CCCCC4)cc3)c3ccccc3-c3ccccc3)cc3ccccc3c2-c2ccc3ccccc3c2C1(C)C]

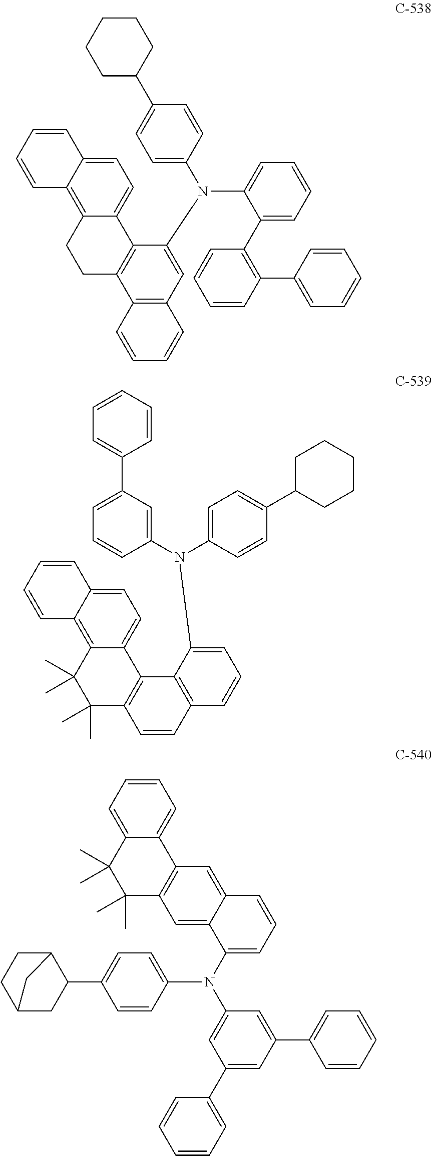

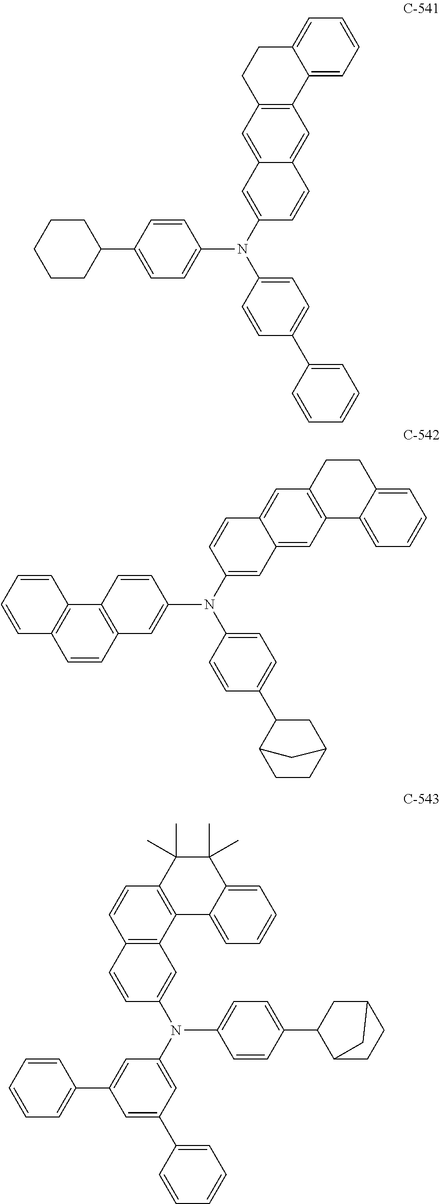

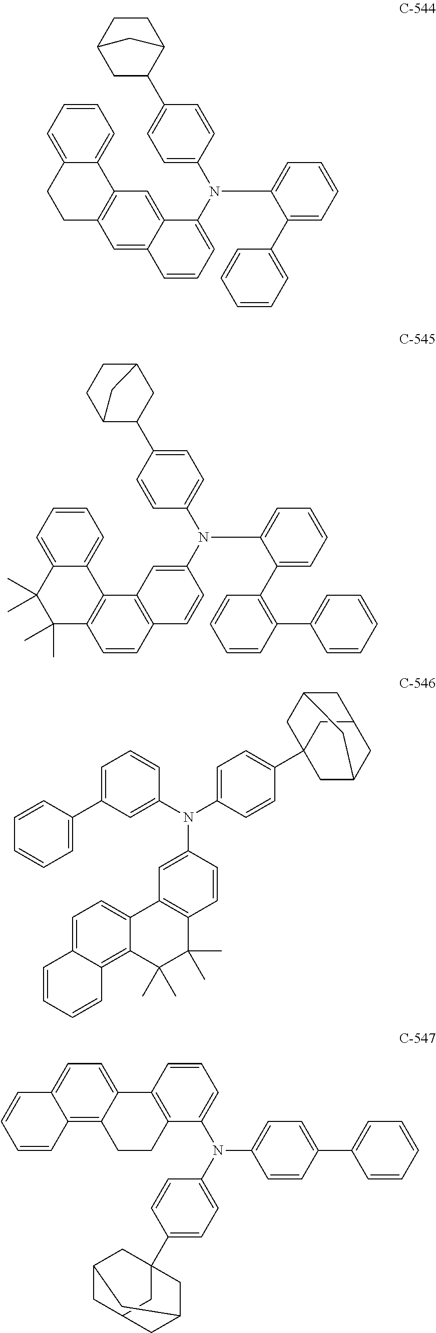

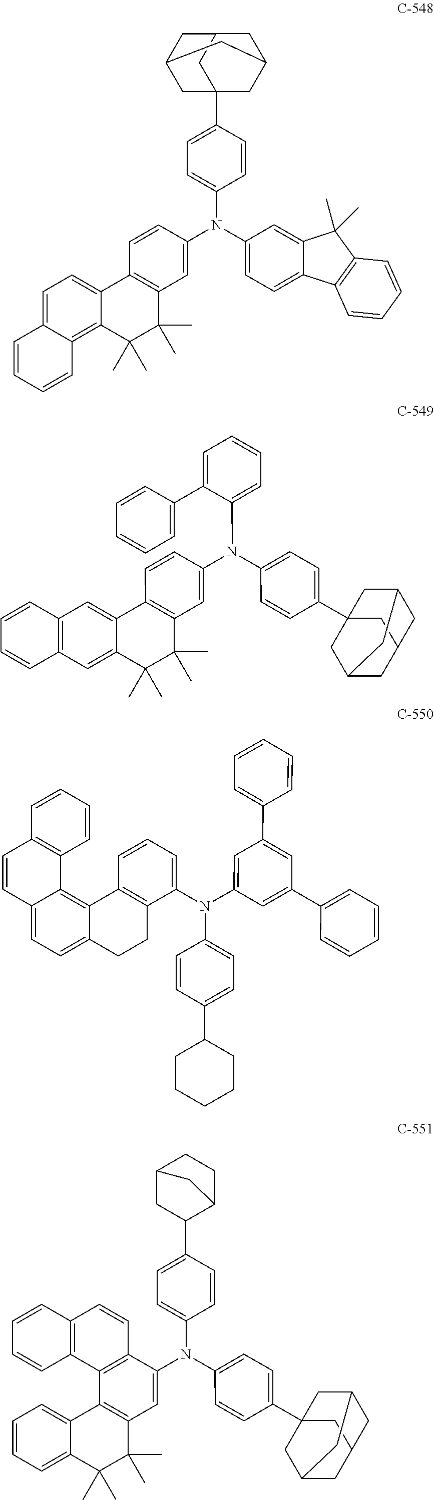 [SMILES: CC1(C)c2ccccc2-c2ccc(N(c3ccc(C45CC6CC(CC(C6)C4)C5)cc3)c3ccc4c(c3)C(C)(C)C(C)(C)c3c-4ccc4ccccc34)cc21 ; CC1(C)c2cc(N(c3ccc(C45CC6CC(CC(C6)C4)C5)cc3)c3ccccc3-c3ccccc3)ccc2-c2cc3ccccc3cc2C1(C)C ; c1ccc(-c2cc(-c3ccccc3)cc(N(c3ccc(C4CCCCC4)cc3)c3cccc4c3CCc3ccc5ccc6ccccc6c5c3-4)c2)cc1 ; CC1(C)c2ccccc2-c2c(cc(N(c3ccc(C4CC5CCC4C5)cc3)c3ccc(C45CC6CC(CC(C6)C4)C5)cc3)c3ccc4ccccc4c23)C1(C)C]

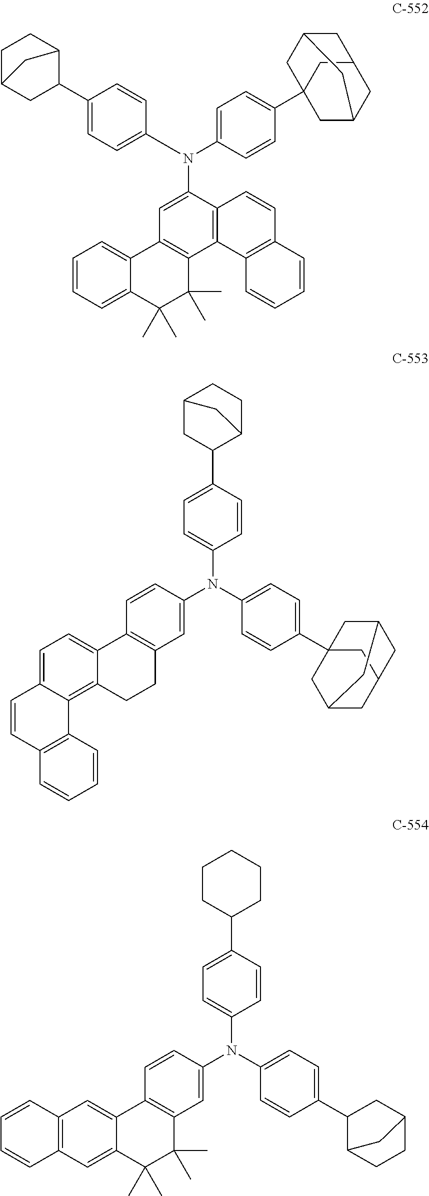 [SMILES: CC1(C)c2ccccc2-c2cc(N(c3ccc(C4CC5CCC4C5)cc3)c3ccc(C45CC6CC(CC(C6)C4)C5)cc3)c3ccc4ccccc4c3c2C1(C)C ; c1ccc2c(c1)ccc1ccc3c(c12)CCc1cc(N(c2ccc(C4CC5CCC4C5)cc2)c2ccc(C45CC6CC(CC(C6)C4)C5)cc2)ccc1-3 ; CC1(C)c2cc(N(c3ccc(C4CCCCC4)cc3)c3ccc(C4CC5CCC4C5)cc3)ccc2-c2cc3ccccc3cc2C1(C)C]

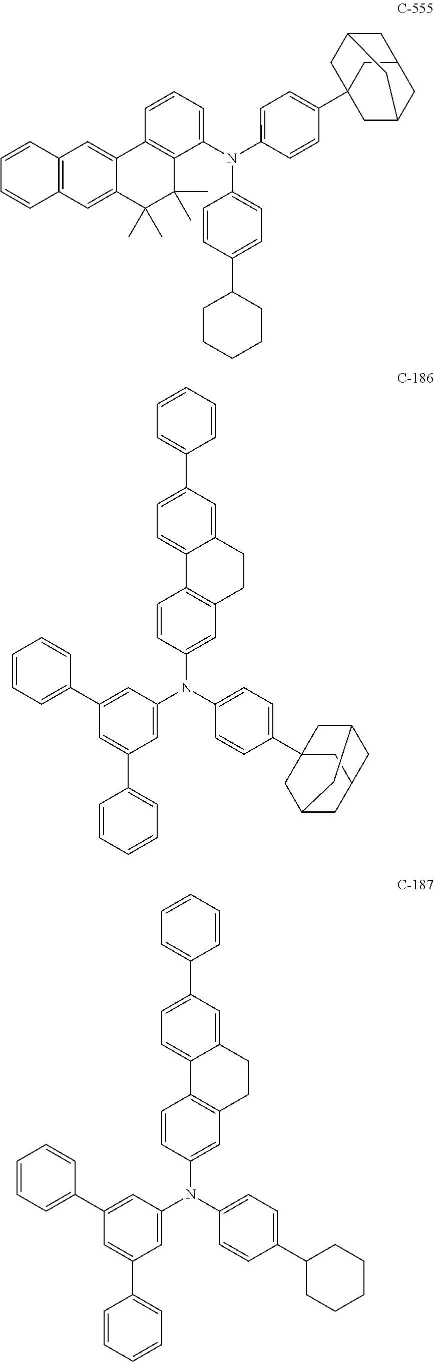 [SMILES: CC1(C)c2cc3ccccc3cc2-c2cccc(N(c3ccc(C4CCCCC4)cc3)c3ccc(C45CC6CC(CC(C6)C4)C5)cc3)c2C1(C)C ; c1ccc(-c2cc(-c3ccccc3)cc(N(c3ccc(C45CC6CC(CC(C6)C4)C5)cc3)c3ccc4c(c3)CCc3cc(-c5ccccc5)ccc3-4)c2)cc1 ; c1ccc(-c2cc(-c3ccccc3)cc(N(c3ccc(C4CCCCC4)cc3)c3ccc4c(c3)CCc3cc(-c5ccccc5)ccc3-4)c2)cc1]

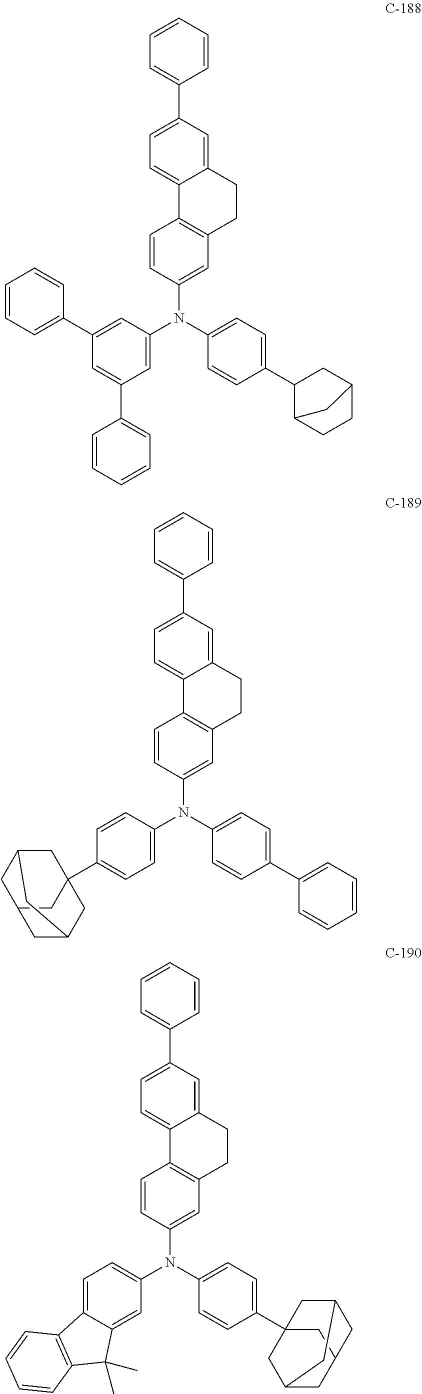 [SMILES: c1ccc(-c2cc(-c3ccccc3)cc(N(c3ccc(C4CC5CCC4C5)cc3)c3ccc4c(c3)CCc3cc(-c5ccccc5)ccc3-4)c2)cc1 ; c1ccc(-c2ccc(N(c3ccc(C45CC6CC(CC(C6)C4)C5)cc3)c3ccc4c(c3)CCc3cc(-c5ccccc5)ccc3-4)cc2)cc1 ; CC1(C)c2ccccc2-c2ccc(N(c3ccc(C45CC6CC(CC(C6)C4)C5)cc3)c3ccc4c(c3)CCc3cc(-c5ccccc5)ccc3-4)cc21]

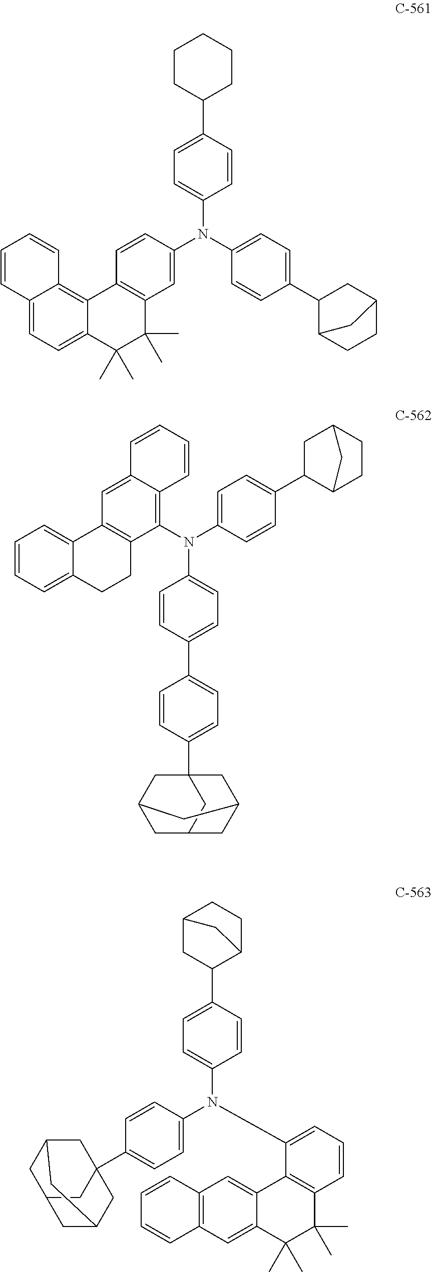 [SMILES: CC1(C)c2cc(N(c3ccc(C4CCCCC4)cc3)c3ccc(C4CC5CCC4C5)cc3)ccc2-c2c(ccc3ccccc23)C1(C)C ; c1ccc2c(c1)CCc1c-2cc2ccccc2c1N(c1ccc(-c2ccc(C34CC5CC(CC(C5)C3)C4)cc2)cc1)c1ccc(C2CC3CCC2C3)cc1 ; CC1(C)c2cc3ccccc3cc2-c2c(N(c3ccc(C4CC5CCC4C5)cc3)c3ccc(C45CC6CC(CC(C6)C4)C5)cc3)cccc2C1(C)C]

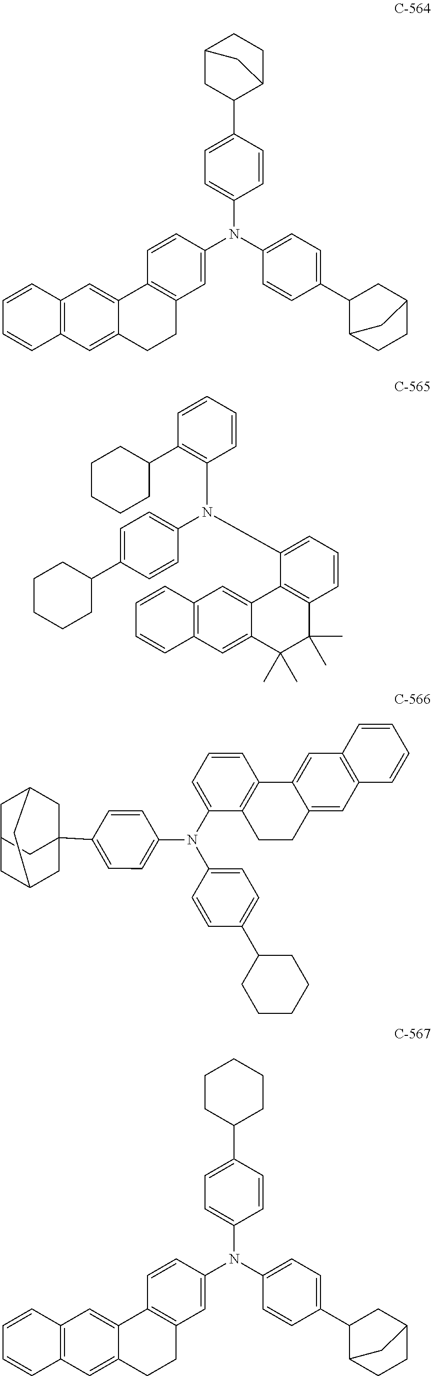 [SMILES: c1ccc2cc3c(cc2c1)CCc1cc(N(c2ccc(C4CC5CCC4C5)cc2)c2ccc(C4CC5CCC4C5)cc2)ccc1-3 ; CC1(C)c2cc3ccccc3cc2-c2c(N(c3ccc(C4CCCCC4)cc3)c3ccccc3C3CCCCC3)cccc2C1(C)C ; c1cc2c(c(N(c3ccc(C4CCCCC4)cc3)c3ccc(C45CC6CC(CC(C6)C4)C5)cc3)c1)CCc1cc3ccccc3cc1-2 ; c1ccc2cc3c(cc2c1)CCc1cc(N(c2ccc(C4CCCCC4)cc2)c2ccc(C4CC5CCC4C5)cc2)ccc1-3]

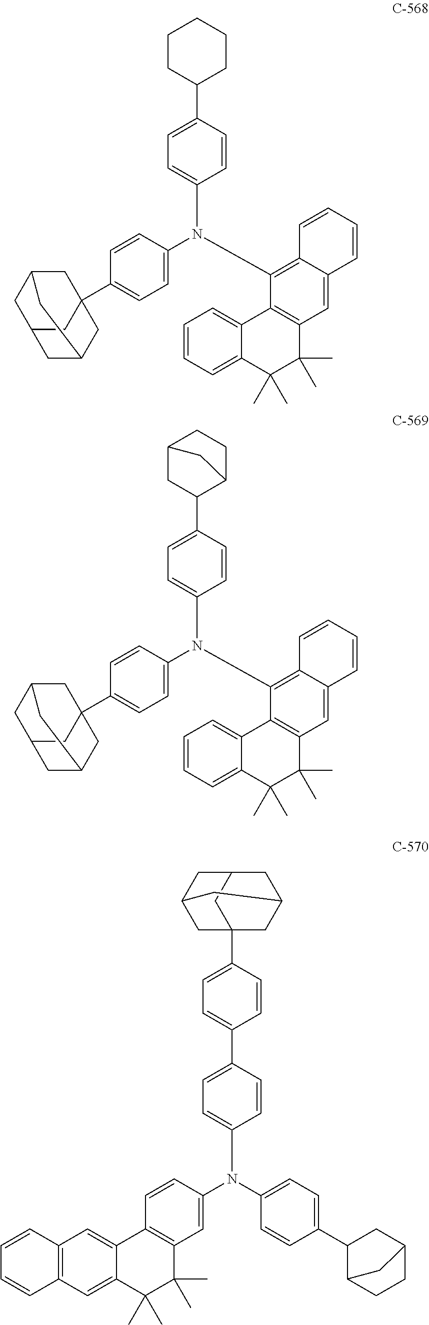 [SMILES: CC1(C)c2ccccc2-c2c(cc3ccccc3c2N(c2ccc(C3CCCCC3)cc2)c2ccc(C34CC5CC(CC(C5)C3)C4)cc2)C1(C)C ; CC1(C)c2ccccc2-c2c(cc3ccccc3c2N(c2ccc(C3CC4CCC3C4)cc2)c2ccc(C34CC5CC(CC(C5)C3)C4)cc2)C1(C)C ; CC1(C)c2cc(N(c3ccc(-c4ccc(C56CC7CC(CC(C7)C5)C6)cc4)cc3)c3ccc(C4CC5CCC4C5)cc3)ccc2-c2cc3ccccc3cc2C1(C)C]

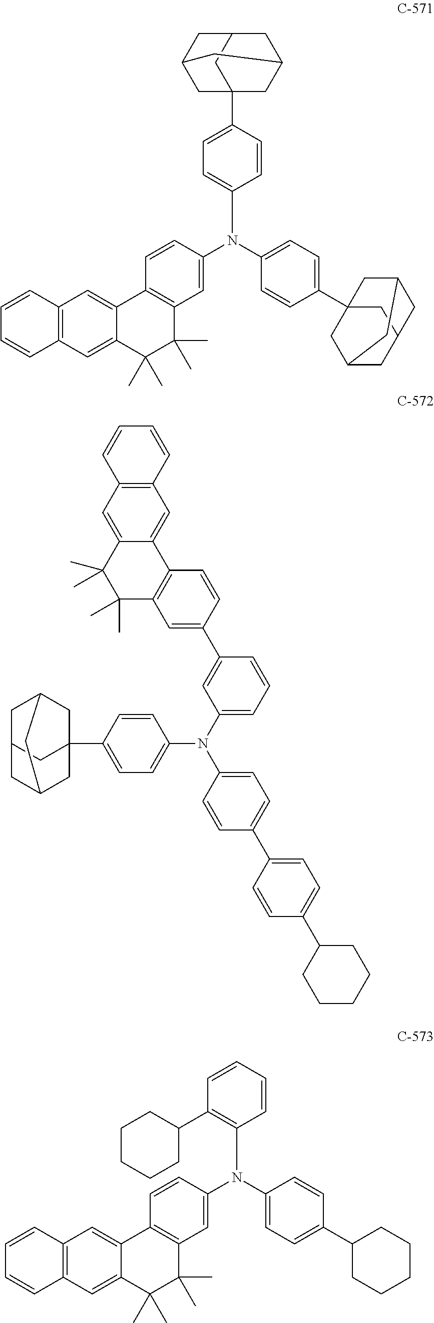

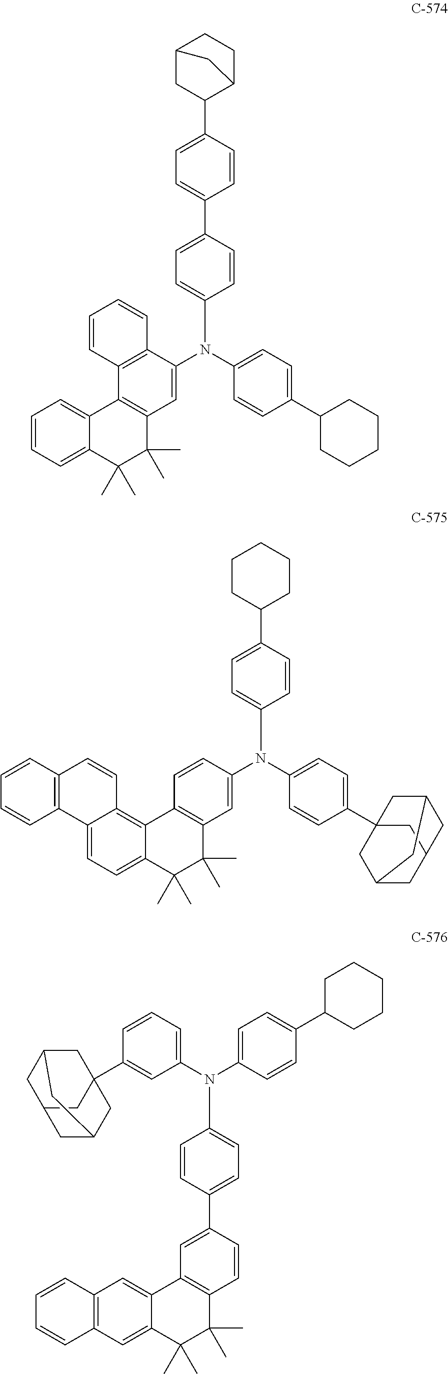 [SMILES: CC1(C)c2ccccc2-c2c(cc(N(c3ccc(-c4ccc(C5CC6CCC5C6)cc4)cc3)c3ccc(C4CCCCC4)cc3)c3ccccc23)C1(C)C ; CC1(C)c2cc(N(c3ccc(C4CCCCC4)cc3)c3ccc(C45CC6CC(CC(C6)C4)C5)cc3)ccc2-c2c(ccc3c2ccc2ccccc23)C1(C)C ; CC1(C)c2ccc(-c3ccc(N(c4ccc(C5CCCCC5)cc4)c4cccc(C56CC7CC(CC(C7)C5)C6)c4)cc3)cc2-c2cc3ccccc3cc2C1(C)C]

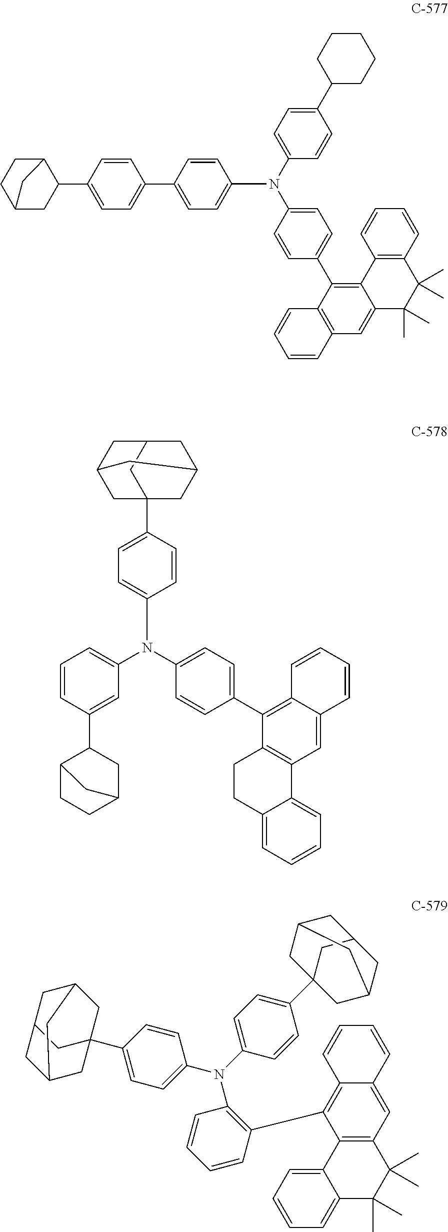 [SMILES: CC1(C)c2ccccc2-c2c(cc3ccccc3c2-c2ccc(N(c3ccc(-c4ccc(C5CC6CCC5C6)cc4)cc3)c3ccc(C4CCCCC4)cc3)cc2)C1(C)C ; c1cc(C2CC3CCC2C3)cc(N(c2ccc(-c3c4c(cc5ccccc35)-c3ccccc3CC4)cc2)c2ccc(C34CC5CC(CC(C5)C3)C4)cc2)c1 ; CC1(C)c2ccccc2-c2c(cc3ccccc3c2-c2ccccc2N(c2ccc(C34CC5CC(CC(C5)C3)C4)cc2)c2ccc(C34CC5CC(CC(C5)C3)C4)cc2)C1(C)C]

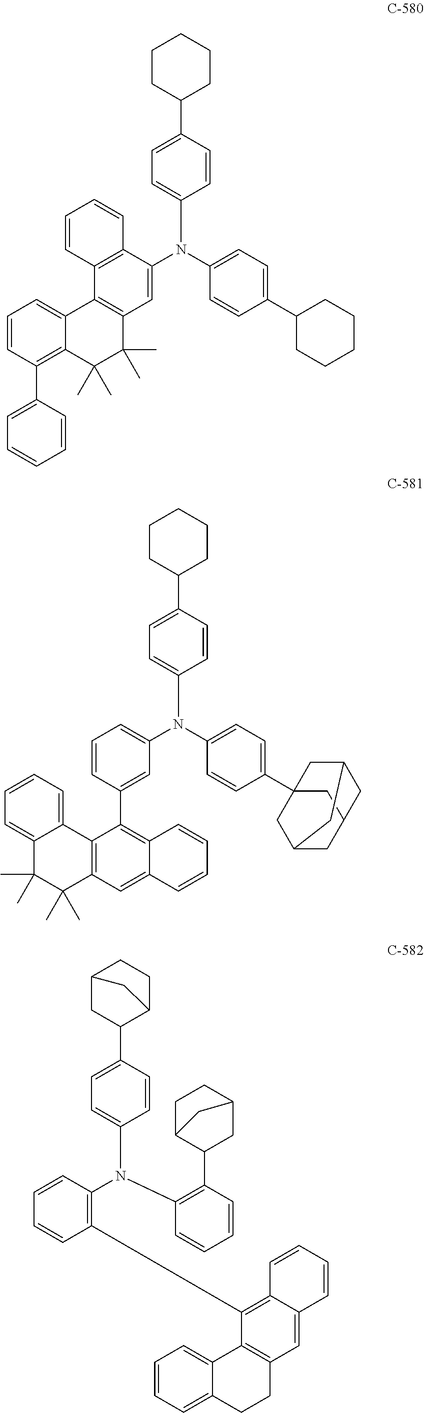 [SMILES: CC1(C)c2cc(N(c3ccc(C4CCCCC4)cc3)c3ccc(C4CCCCC4)cc3)c3ccccc3c2-c2cccc(-c3ccccc3)c2C1(C)C ; CC1(C)c2ccccc2-c2c(cc3ccccc3c2-c2cccc(N(c3ccc(C4CCCCC4)cc3)c3ccc(C45CC6CC(CC(C6)C4)C5)cc3)c2)C1(C)C ; c1ccc2c(c1)CCc1cc3ccccc3c(-c3ccccc3N(c3ccc(C4CC5CCC4C5)cc3)c3ccccc3C3CC4CCC3C4)c1-2]

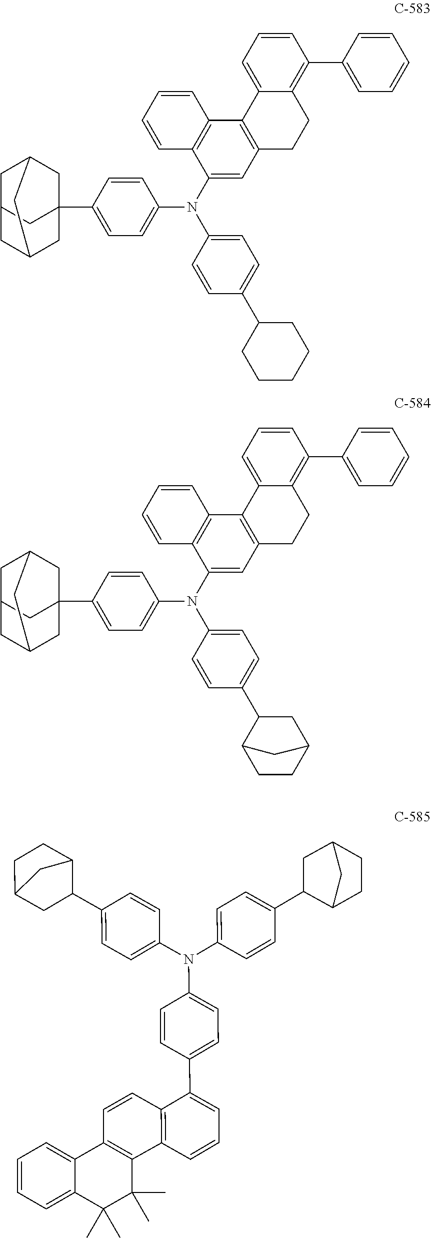 [SMILES: c1ccc(-c2cccc3c2CCc2cc(N(c4ccc(C5CCCCC5)cc4)c4ccc(C56CC7CC(CC(C7)C5)C6)cc4)c4ccccc4c2-3)cc1 ; c1ccc(-c2cccc3c2CCc2cc(N(c4ccc(C5CC6CCC5C6)cc4)c4ccc(C56CC7CC(CC(C7)C5)C6)cc4)c4ccccc4c2-3)cc1 ; CC1(C)c2ccccc2-c2ccc3c(-c4ccc(N(c5ccc(C6CC7CCC6C7)cc5)c5ccc(C6CC7CCC6C7)cc5)cc4)cccc3c2C1(C)C]

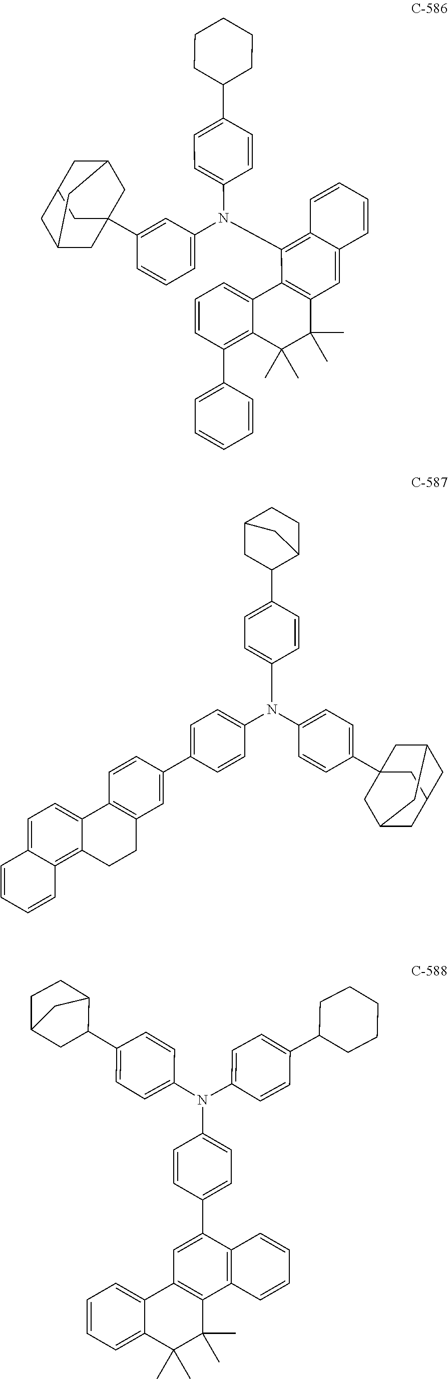 [SMILES: CC1(C)c2cc3ccccc3c(N(c3ccc(C4CCCCC4)cc3)c3cccc(C45CC6CC(CC(C6)C4)C5)c3)c2-c2cccc(-c3ccccc3)c2C1(C)C ; c1ccc2c3c(ccc2c1)-c1ccc(-c2ccc(N(c4ccc(C5CC6CCC5C6)cc4)c4ccc(C56CC7CC(CC(C7)C5)C6)cc4)cc2)cc1CC3 ; CC1(C)c2ccccc2-c2cc(-c3ccc(N(c4ccc(C5CCCCC5)cc4)c4ccc(C5CC6CCC5C6)cc4)cc3)c3ccccc3c2C1(C)C]

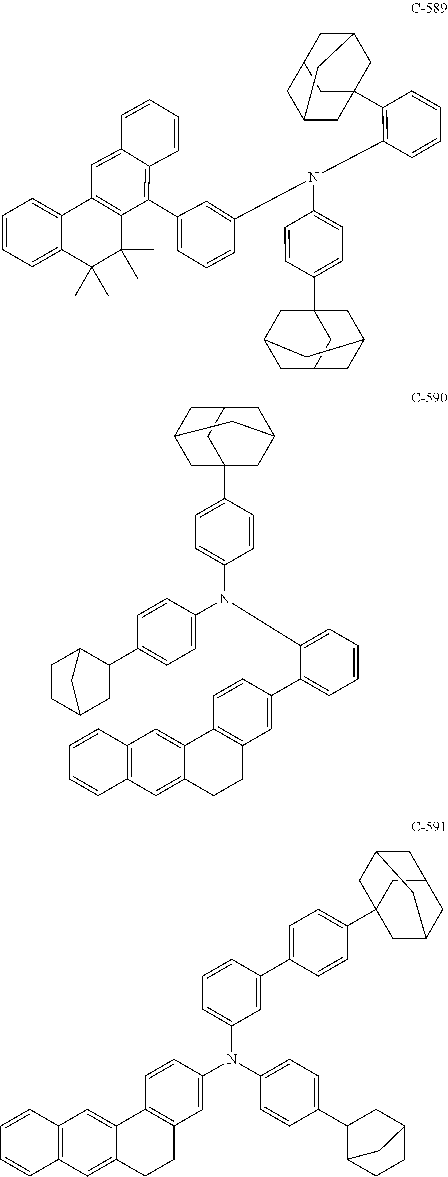

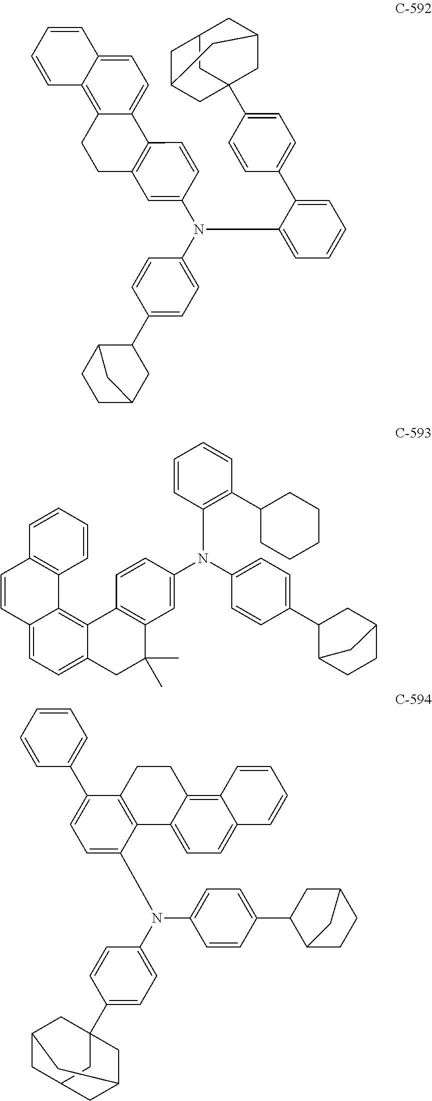

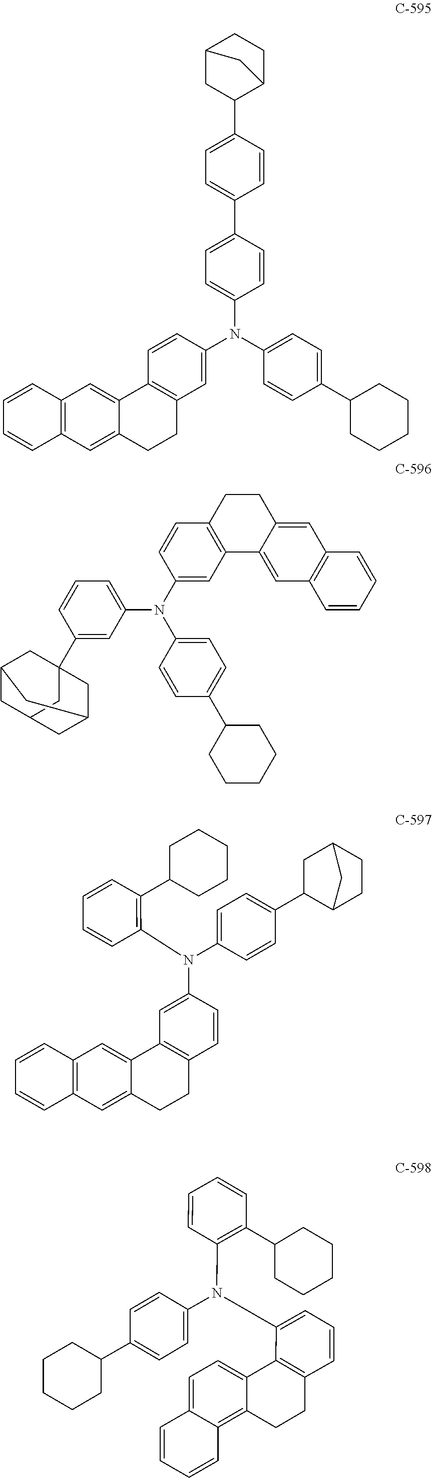 [SMILES: c1ccc2cc3c(cc2c1)CCc1cc(N(c2ccc(-c4ccc(C5CC6CCC5C6)cc4)cc2)c2ccc(C4CCCCC4)cc2)ccc1-3 ; c1cc(N(c2ccc(C3CCCCC3)cc2)c2ccc3c(c2)-c2cc4ccccc4cc2CC3)cc(C23CC4CC(CC(C4)C2)C3)c1 ; c1ccc(N(c2ccc(C3CC4CCC3C4)cc2)c2ccc3c(c2)-c2cc4ccccc4cc2CC3)c(C2CCCCC2)c1 ; c1ccc(N(c2ccc(C3CCCCC3)cc2)c2cccc3c2-c2ccc4ccccc4c2CC3)c(C2CCCCC2)c1]

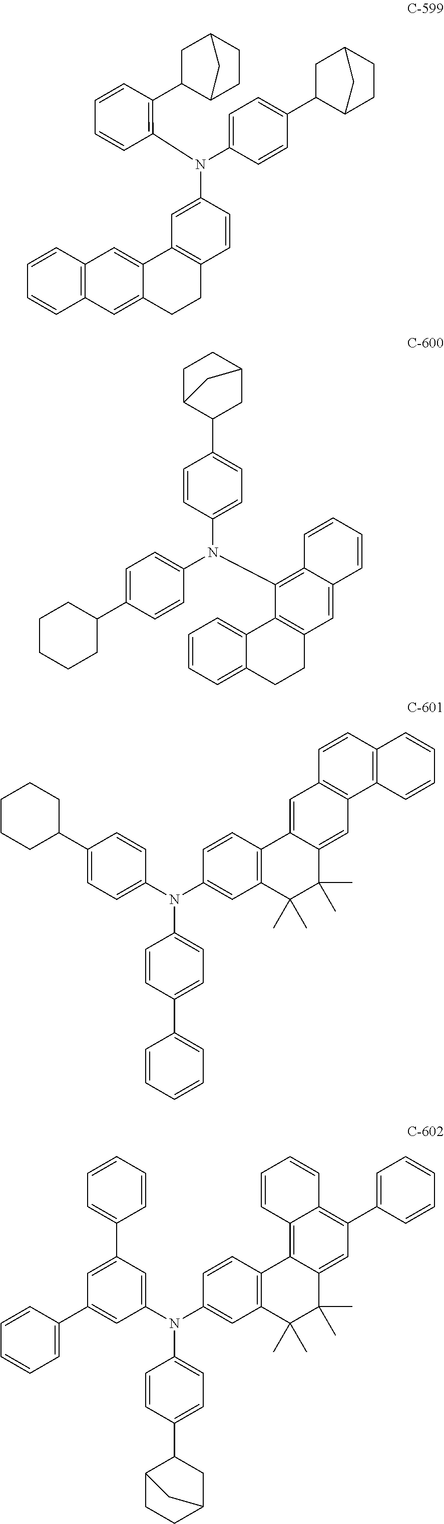 [SMILES: c1ccc(N(c2ccc(C3CC4CCC3C4)cc2)c2ccc3c(c2)-c2cc4ccccc4cc2CC3)c(C2CC3CCC2C3)c1 ; c1ccc2c(c1)CCc1cc3ccccc3c(N(c3ccc(C4CCCCC4)cc3)c3ccc(C4CC5CCC4C5)cc3)c1-2 ; CC1(C)c2cc(N(c3ccc(-c4ccccc4)cc3)c3ccc(C4CCCCC4)cc3)ccc2-c2cc3ccc4ccccc4c3cc2C1(C)C ; CC1(C)c2cc(N(c3ccc(C4CC5CCC4C5)cc3)c3cc(-c4ccccc4)cc(-c4ccccc4)c3)ccc2-c2c(cc(-c3ccccc3)c3ccccc23)C1(C)C]

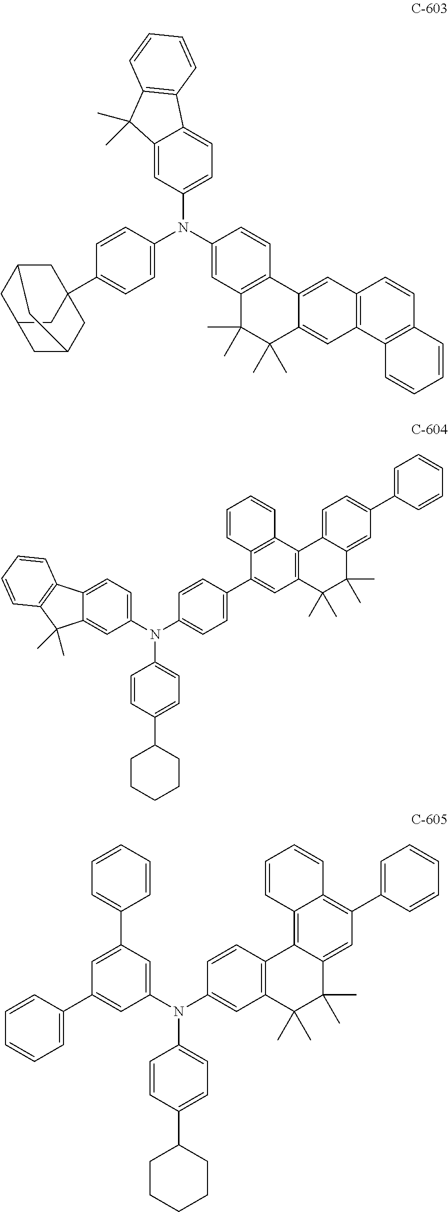 [SMILES: CC1(C)c2ccccc2-c2ccc(N(c3ccc(C45CC6CC(CC(C6)C4)C5)cc3)c3ccc4c(c3)C(C)(C)C(C)(C)c3cc5c(ccc6ccccc65)cc3-4)cc21 ; CC1(C)c2ccccc2-c2ccc(N(c3ccc(-c4cc5c(c6ccccc46)-c4ccc(-c6ccccc6)cc4C(C)(C)C5(C)C)cc3)c3ccc(C4CCCCC4)cc3)cc21 ; CC1(C)c2cc(N(c3ccc(C4CCCCC4)cc3)c3cc(-c4ccccc4)cc(-c4ccccc4)c3)ccc2-c2c(cc(-c3ccccc3)c3ccccc23)C1(C)C]

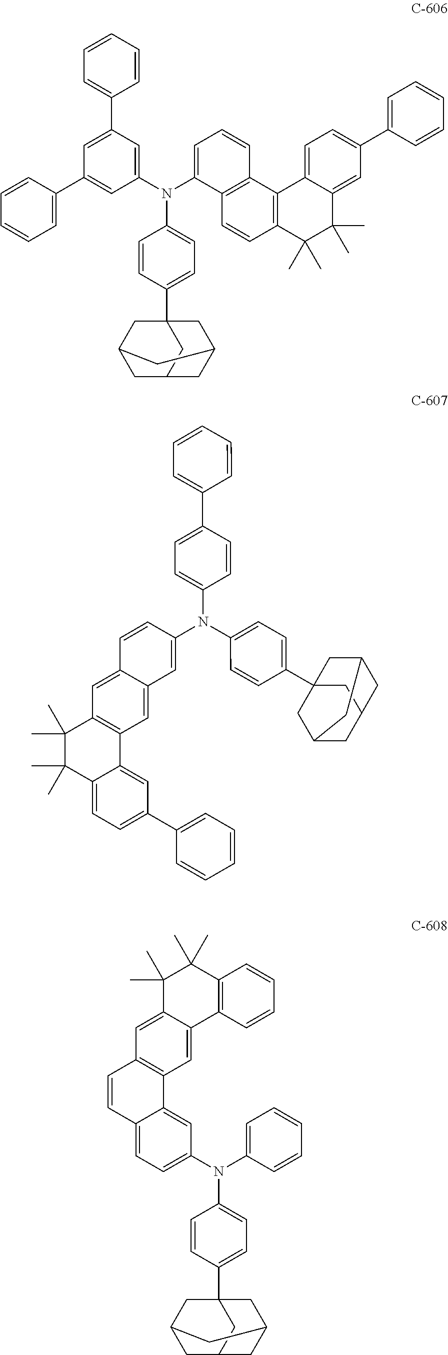 [SMILES: CC1(C)c2cc(-c3ccccc3)ccc2-c2c(ccc3c(N(c4ccc(C56CC7CC(CC(C7)C5)C6)cc4)c4cc(-c5ccccc5)cc(-c5ccccc5)c4)cccc23)C1(C)C ; CC1(C)c2ccc(-c3ccccc3)cc2-c2cc3cc(N(c4ccc(-c5ccccc5)cc4)c4ccc(C56CC7CC(CC(C7)C5)C6)cc4)ccc3cc2C1(C)C ; CC1(C)c2ccccc2-c2cc3c(ccc4ccc(N(c5ccccc5)c5ccc(C67CC8CC(CC(C8)C6)C7)cc5)cc43)cc2C1(C)C]

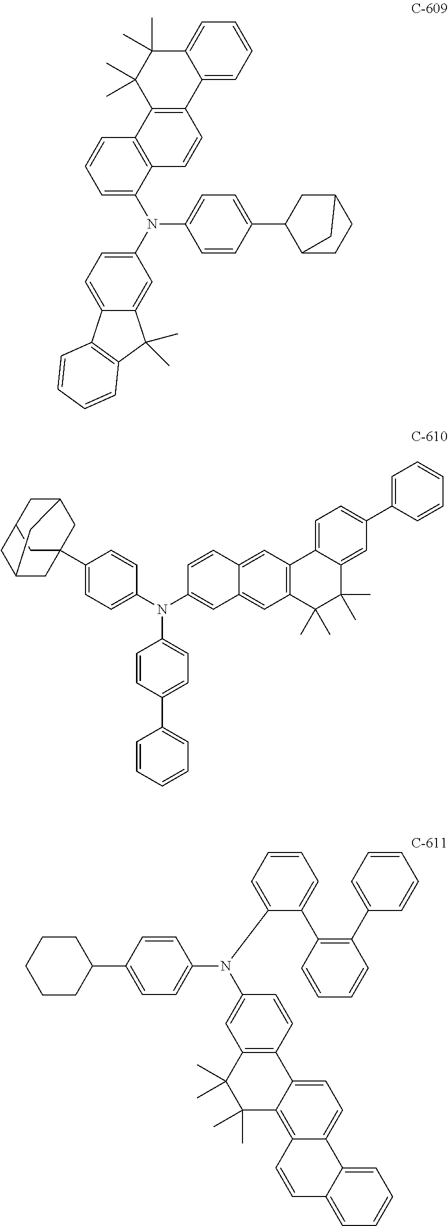 [SMILES: CC1(C)c2ccccc2-c2ccc(N(c3ccc(C4CC5CCC4C5)cc3)c3cccc4c5c(ccc34)-c3ccccc3C(C)(C)C5(C)C)cc21 ; CC1(C)c2cc(-c3ccccc3)ccc2-c2cc3ccc(N(c4ccc(-c5ccccc5)cc4)c4ccc(C56CC7CC(CC(C7)C5)C6)cc4)cc3cc2C1(C)C ; CC1(C)c2cc(N(c3ccc(C4CCCCC4)cc3)c3ccccc3-c3ccccc3-c3ccccc3)ccc2-c2ccc3c(ccc4ccccc43)c2C1(C)C]

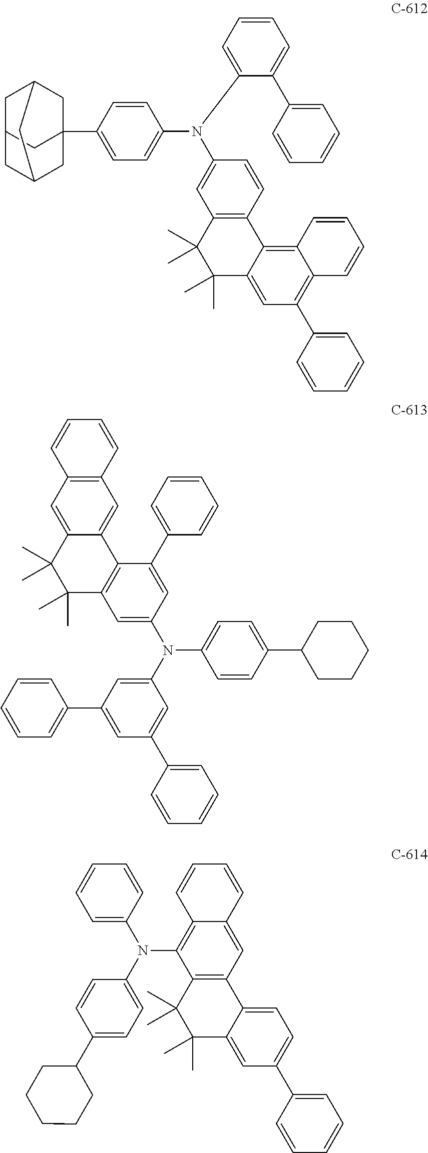

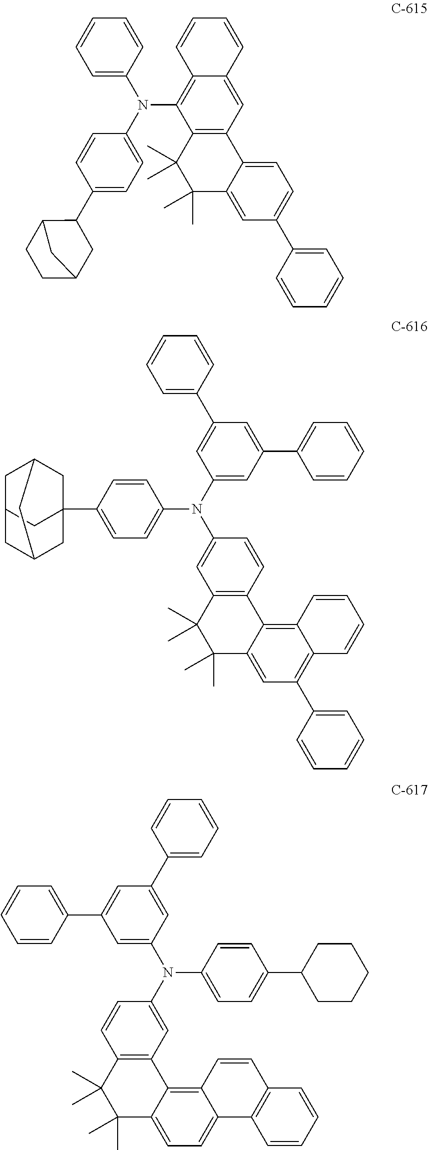 [SMILES: CC1(C)c2cc(-c3ccccc3)ccc2-c2cc3ccccc3c(N(c3ccccc3)c3ccc(C4CC5CCC4C5)cc3)c2C1(C)C ; CC1(C)c2cc(N(c3ccc(C45CC6CC(CC(C6)C4)C5)cc3)c3cc(-c4ccccc4)cc(-c4ccccc4)c3)ccc2-c2c(cc(-c3ccccc3)c3ccccc23)C1(C)C ; CC1(C)c2ccc(N(c3ccc(C4CCCCC4)cc3)c3cc(-c4ccccc4)cc(-c4ccccc4)c3)cc2-c2c(ccc3c2ccc2ccccc23)C1(C)C]

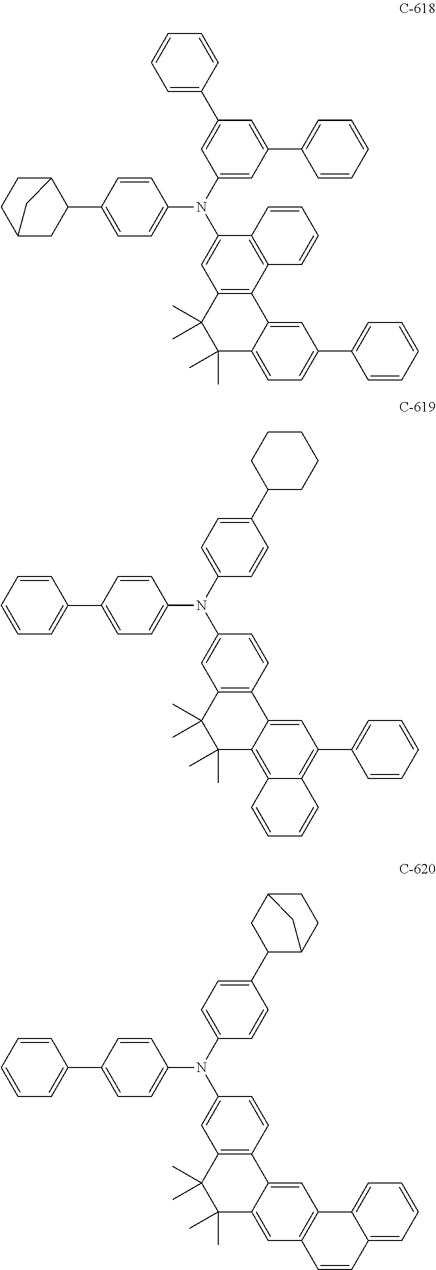 [SMILES: CC1(C)c2ccc(-c3ccccc3)cc2-c2c(cc(N(c3ccc(C4CC5CCC4C5)cc3)c3cc(-c4ccccc4)cc(-c4ccccc4)c3)c3ccccc23)C1(C)C ; CC1(C)c2cc(N(c3ccc(-c4ccccc4)cc3)c3ccc(C4CCCCC4)cc3)ccc2-c2cc(-c3ccccc3)c3ccccc3c2C1(C)C ; CC1(C)c2cc(N(c3ccc(-c4ccccc4)cc3)c3ccc(C4CC5CCC4C5)cc3)ccc2-c2cc3c(ccc4ccccc43)cc2C1(C)C]

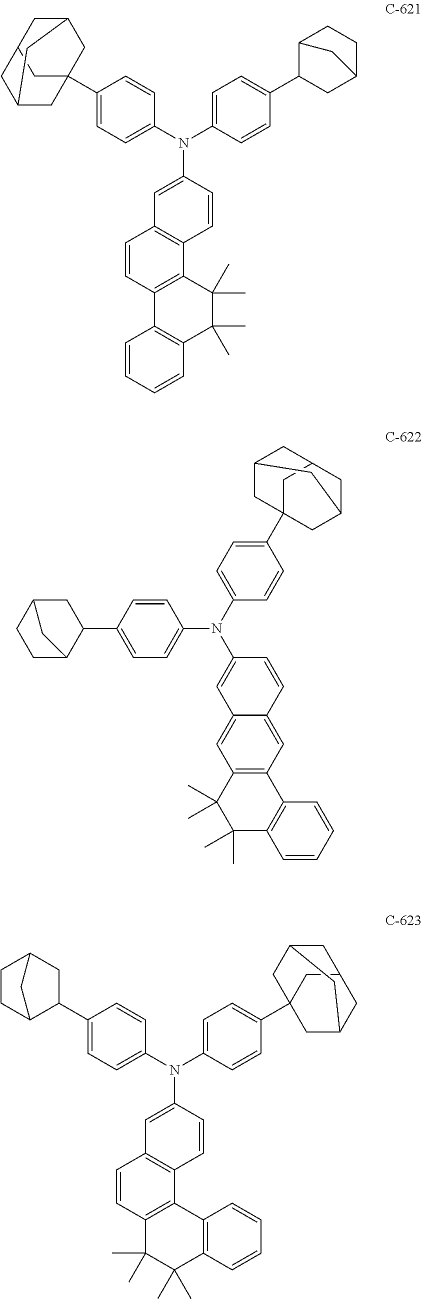 [SMILES: CC1(C)c2ccccc2-c2ccc3cc(N(c4ccc(C5CC6CCC5C6)cc4)c4ccc(C56CC7CC(CC(C7)C5)C6)cc4)ccc3c2C1(C)C ; CC1(C)c2ccccc2-c2cc3ccc(N(c4ccc(C5CC6CCC5C6)cc4)c4ccc(C56CC7CC(CC(C7)C5)C6)cc4)cc3cc2C1(C)C ; CC1(C)c2ccccc2-c2c(ccc3cc(N(c4ccc(C5CC6CCC5C6)cc4)c4ccc(C56CC7CC(CC(C7)C5)C6)cc4)ccc23)C1(C)C]

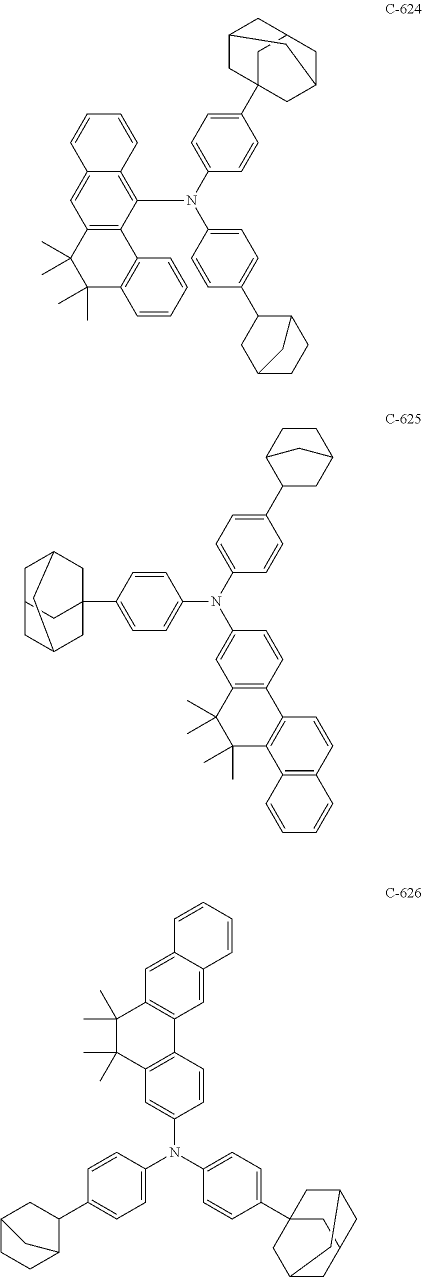 [SMILES: CC1(C)c2ccccc2-c2c(cc3ccccc3c2N(c2ccc(C3CC4CCC3C4)cc2)c2ccc(C34CC5CC(CC(C5)C3)C4)cc2)C1(C)C ; CC1(C)c2cc(N(c3ccc(C4CC5CCC4C5)cc3)c3ccc(C45CC6CC(CC(C6)C4)C5)cc3)ccc2-c2ccc3ccccc3c2C1(C)C ; CC1(C)c2cc(N(c3ccc(C4CC5CCC4C5)cc3)c3ccc(C45CC6CC(CC(C6)C4)C5)cc3)ccc2-c2cc3ccccc3cc2C1(C)C]

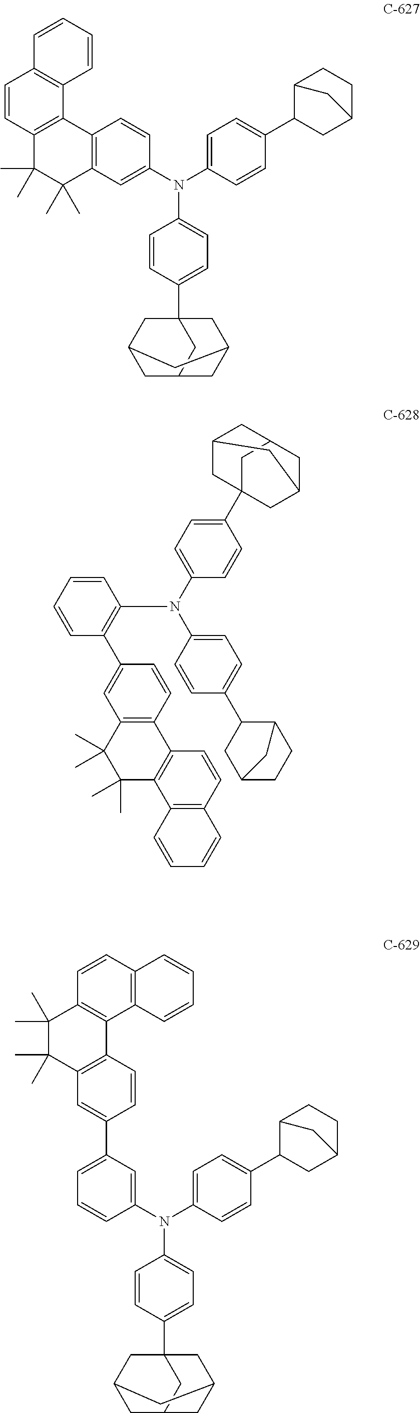 [SMILES: CC1(C)c2cc(N(c3ccc(C4CC5CCC4C5)cc3)c3ccc(C45CC6CC(CC(C6)C4)C5)cc3)ccc2-c2c(ccc3ccccc23)C1(C)C ; CC1(C)c2cc(-c3ccccc3N(c3ccc(C4CC5CCC4C5)cc3)c3ccc(C45CC6CC(CC(C6)C4)C5)cc3)ccc2-c2ccc3ccccc3c2C1(C)C ; CC1(C)c2cc(-c3cccc(N(c4ccc(C5CC6CCC5C6)cc4)c4ccc(C56CC7CC(CC(C7)C5)C6)cc4)c3)ccc2-c2c(ccc3ccccc23)C1(C)C]

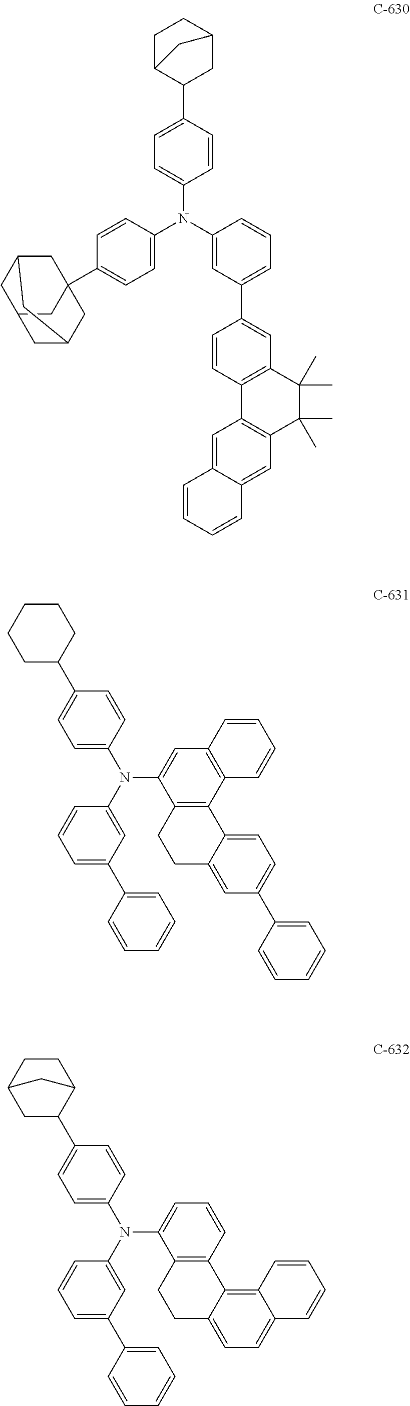 [SMILES: CC1(C)c2cc(-c3cccc(N(c4ccc(C5CC6CCC5C6)cc4)c4ccc(C56CC7CC(CC(C7)C5)C6)cc4)c3)ccc2-c2cc3ccccc3cc2C1(C)C ; c1ccc(-c2cccc(N(c3ccc(C4CCCCC4)cc3)c3cc4ccccc4c4c3CCc3cc(-c5ccccc5)ccc3-4)c2)cc1 ; c1ccc(-c2cccc(N(c3ccc(C4CC5CCC4C5)cc3)c3cccc4c3CCc3ccc5ccccc5c3-4)c2)cc1]

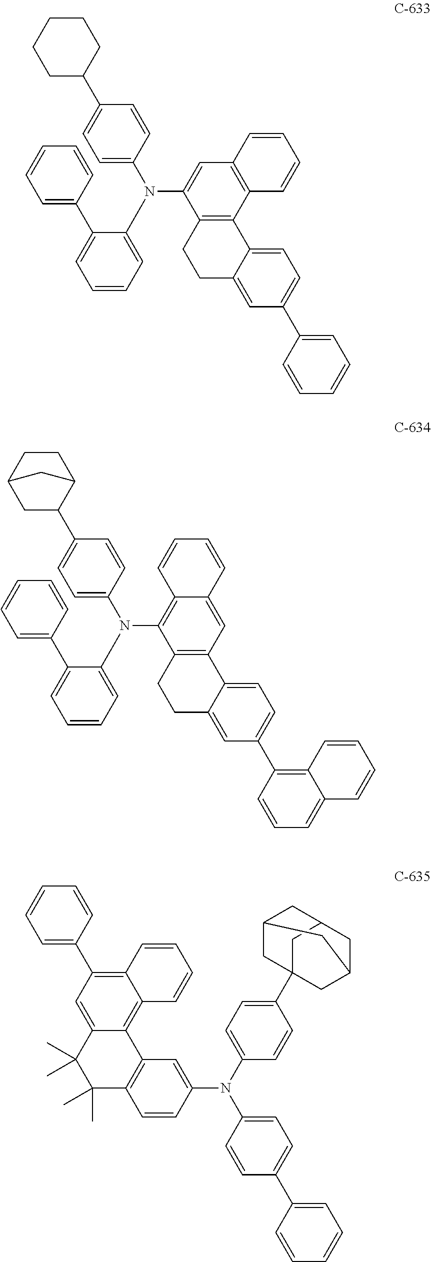 [SMILES: c1ccc(-c2ccc3c(c2)CCc2c(N(c4ccc(C5CCCCC5)cc4)c4ccccc4-c4ccccc4)cc4ccccc4c2-3)cc1 ; c1ccc(-c2ccccc2N(c2ccc(C3CC4CCC3C4)cc2)c2c3c(cc4ccccc24)-c2ccc(-c4cccc5ccccc45)cc2CC3)cc1 ; CC1(C)c2ccc(N(c3ccc(-c4ccccc4)cc3)c3ccc(C45CC6CC(CC(C6)C4)C5)cc3)cc2-c2c(cc(-c3ccccc3)c3ccccc23)C1(C)C]

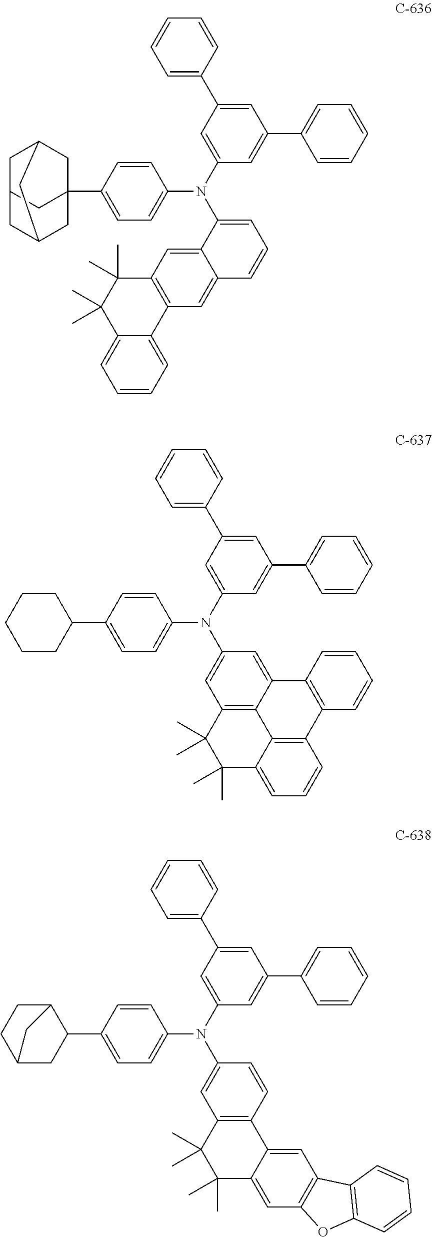 [SMILES: CC1(C)c2ccccc2-c2cc3cccc(N(c4ccc(C56CC7CC(CC(C7)C5)C6)cc4)c4cc(-c5ccccc5)cc(-c5ccccc5)c4)c3cc2C1(C)C ; CC1(C)c2cccc3c4ccccc4c4cc(N(c5ccc(C6CCCCC6)cc5)c5cc(-c6ccccc6)cc(-c6ccccc6)c5)cc(c4c23)C1(C)C ; CC1(C)c2cc(N(c3ccc(C4CC5CCC4C5)cc3)c3cc(-c4ccccc4)cc(-c4ccccc4)c3)ccc2-c2cc3c(cc2C1(C)C)oc1ccccc13]

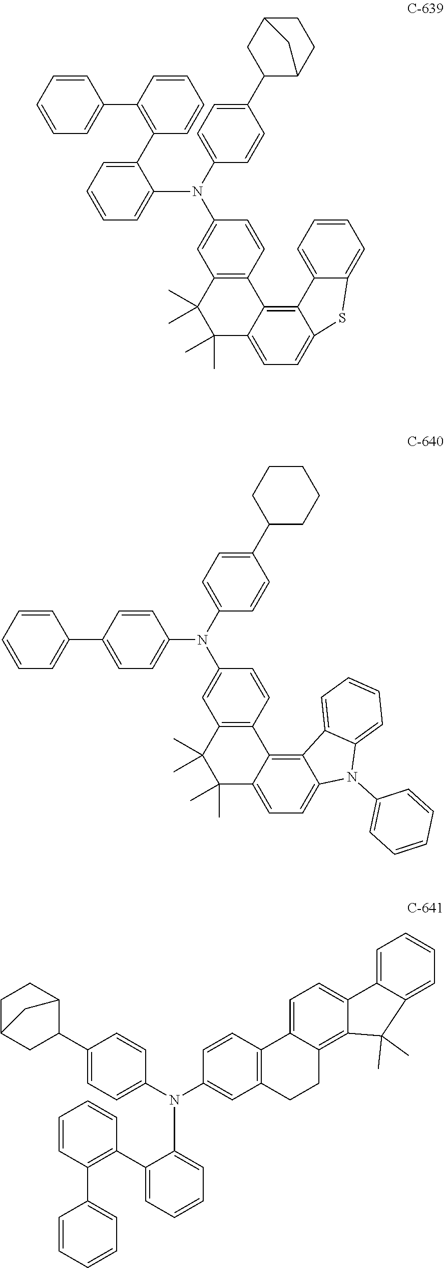 [SMILES: CC1(C)c2cc(N(c3ccc(C4CC5CCC4C5)cc3)c3ccccc3-c3ccccc3-c3ccccc3)ccc2-c2c(ccc3sc4ccccc4c23)C1(C)C ; CC1(C)c2cc(N(c3ccc(-c4ccccc4)cc3)c3ccc(C4CCCCC4)cc3)ccc2-c2c(ccc3c2c2ccccc2n3-c2ccccc2)C1(C)C ; CC1(C)c2ccccc2-c2ccc3c(c21)CCc1cc(N(c2ccc(C4CC5CCC4C5)cc2)c2ccccc2-c2ccccc2-c2ccccc2)ccc1-3]

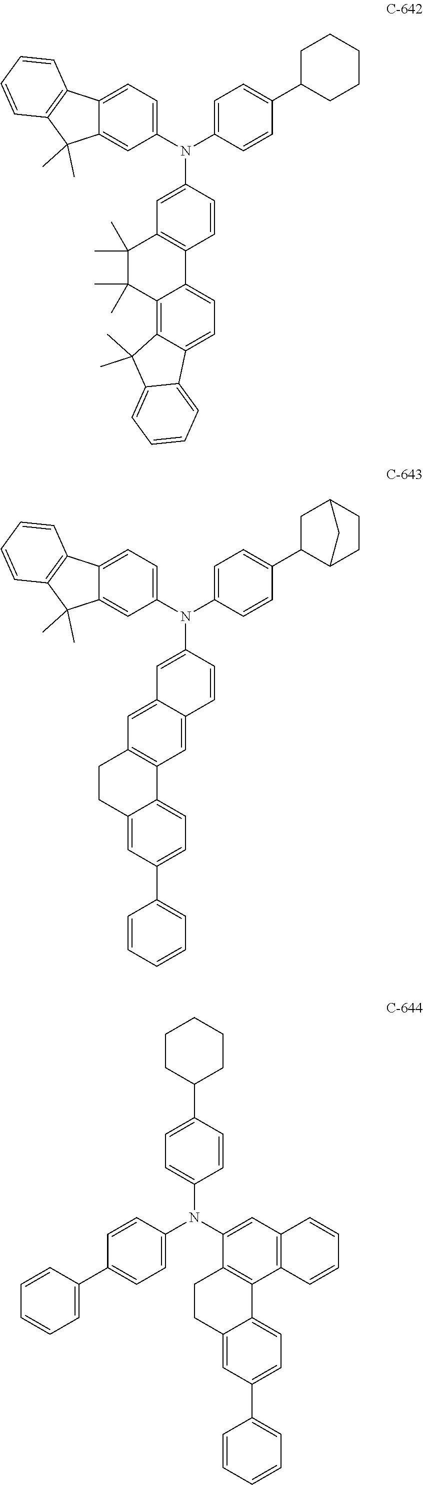 [SMILES: CC1(C)c2ccccc2-c2ccc(N(c3ccc(C4CCCCC4)cc3)c3ccc4c(c3)C(C)(C)C(C)(C)c3c-4ccc4c3C(C)(C)c3ccccc3-4)cc21 ; CC1(C)c2ccccc2-c2ccc(N(c3ccc(C4CC5CCC4C5)cc3)c3ccc4cc5c(cc4c3)CCc3cc(-c4ccccc4)ccc3-5)cc21 ; c1ccc(-c2ccc(N(c3ccc(C4CCCCC4)cc3)c3cc4ccccc4c4c3CCc3cc(-c5ccccc5)ccc3-4)cc2)cc1]

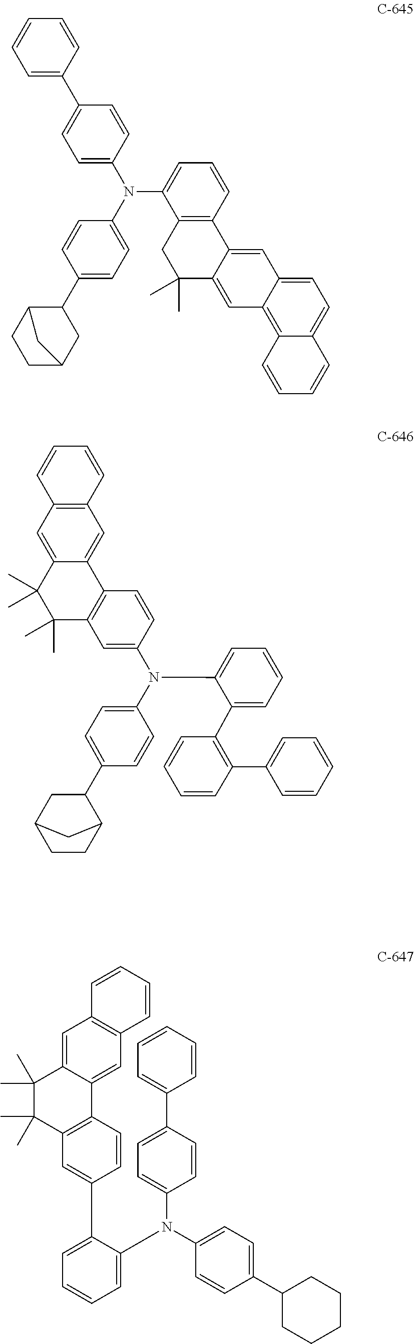 [SMILES: CC1(C)Cc2c(cccc2N(c2ccc(-c3ccccc3)cc2)c2ccc(C3CC4CCC3C4)cc2)-c2cc3ccc4ccccc4c3cc21 ; CC1(C)c2cc(N(c3ccc(C4CC5CCC4C5)cc3)c3ccccc3-c3ccccc3-c3ccccc3)ccc2-c2cc3ccccc3cc2C1(C)C ; CC1(C)c2cc(-c3ccccc3N(c3ccc(-c4ccccc4)cc3)c3ccc(C4CCCCC4)cc3)ccc2-c2cc3ccccc3cc2C1(C)C]

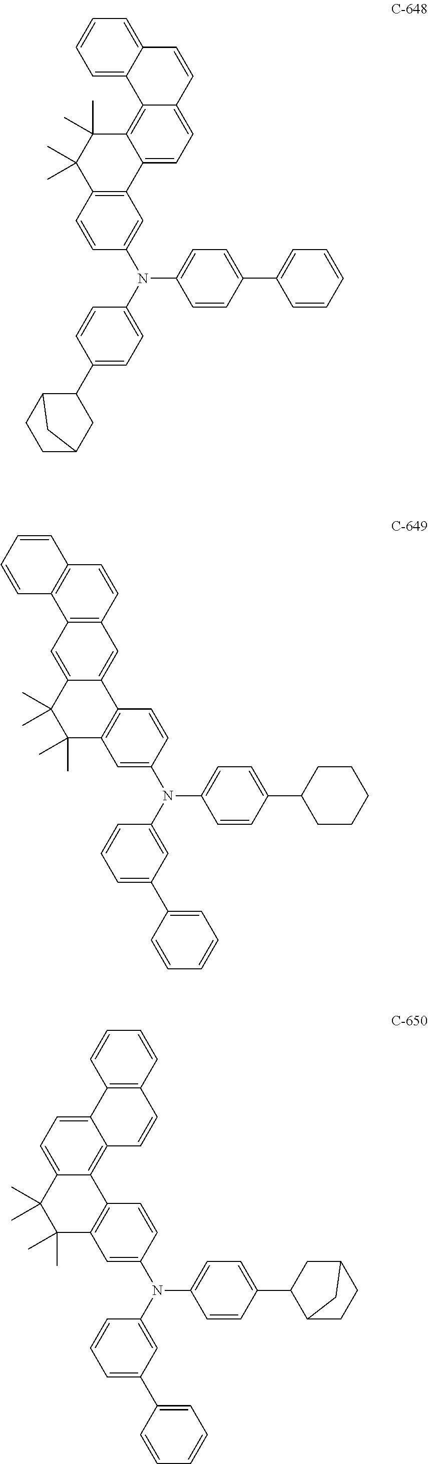 [SMILES: CC1(C)c2ccc(N(c3ccc(-c4ccccc4)cc3)c3ccc(C4CC5CCC4C5)cc3)cc2-c2ccc3ccc4ccccc4c3c2C1(C)C ; CC1(C)c2cc(N(c3ccc(C4CCCCC4)cc3)c3cccc(-c4ccccc4)c3)ccc2-c2cc3ccc4ccccc4c3cc2C1(C)C ; CC1(C)c2cc(N(c3ccc(C4CC5CCC4C5)cc3)c3cccc(-c4ccccc4)c3)ccc2-c2c(ccc3c2ccc2ccccc23)C1(C)C]

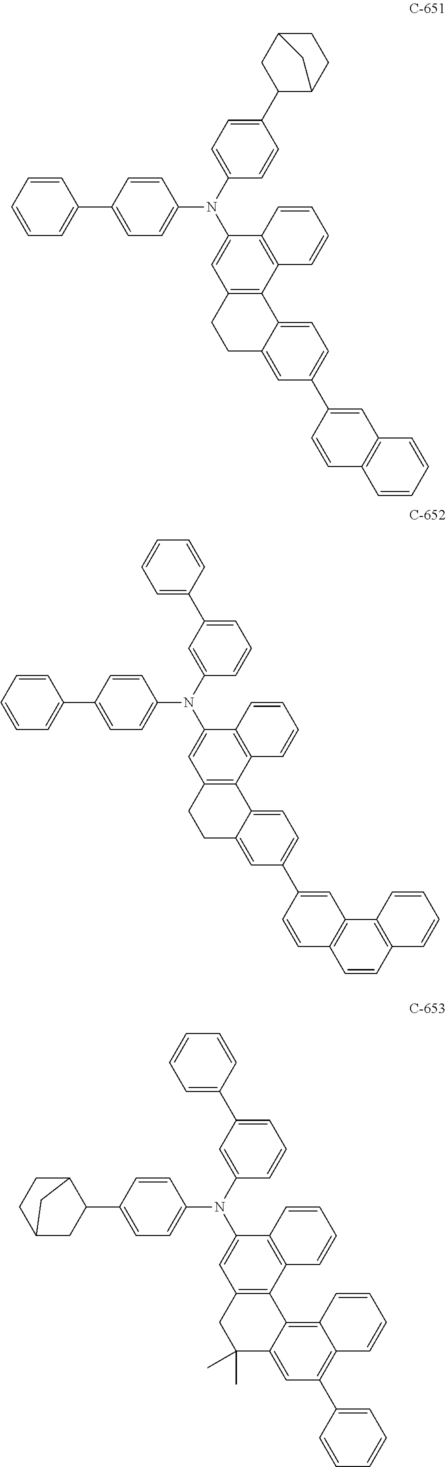 [SMILES: c1ccc(-c2ccc(N(c3ccc(C4CC5CCC4C5)cc3)c3cc4c(c5ccccc35)-c3ccc(-c5ccc6ccccc6c5)cc3CC4)cc2)cc1 ; c1ccc(-c2ccc(N(c3cccc(-c4ccccc4)c3)c3cc4c(c5ccccc35)-c3ccc(-c5ccc6ccc7ccccc7c6c5)cc3CC4)cc2)cc1 ; CC1(C)Cc2cc(N(c3ccc(C4CC5CCC4C5)cc3)c3cccc(-c4ccccc4)c3)c3ccccc3c2-c2c1cc(-c1ccccc1)c1ccccc21]

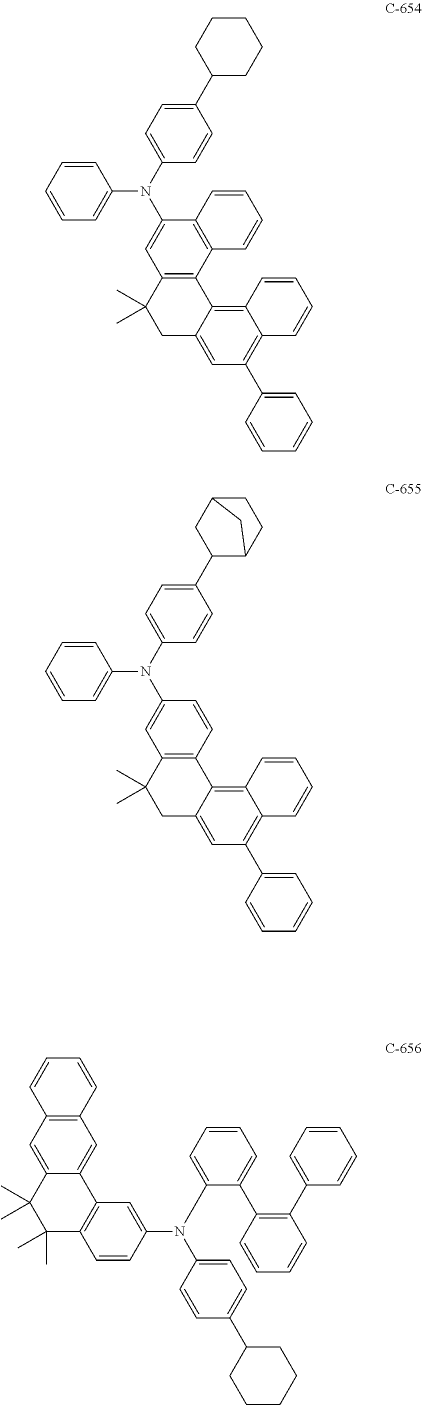 [SMILES: CC1(C)Cc2cc(-c3ccccc3)c3ccccc3c2-c2c1cc(N(c1ccccc1)c1ccc(C3CCCCC3)cc1)c1ccccc21 ; CC1(C)Cc2cc(-c3ccccc3)c3ccccc3c2-c2ccc(N(c3ccccc3)c3ccc(C4CC5CCC4C5)cc3)cc21 ; CC1(C)c2ccc(N(c3ccc(C4CCCCC4)cc3)c3ccccc3-c3ccccc3-c3ccccc3)cc2-c2cc3ccccc3cc2C1(C)C]

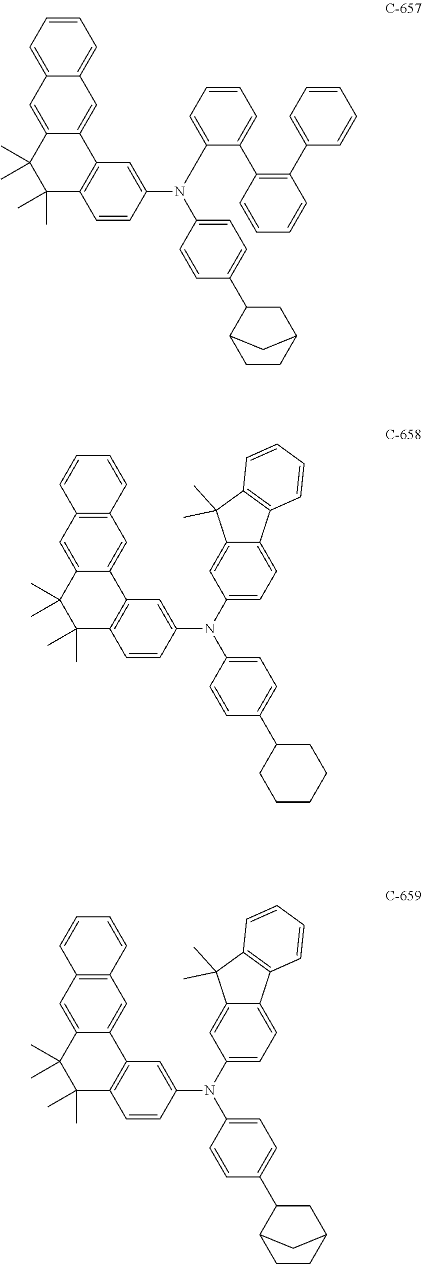 [SMILES: CC1(C)c2ccc(N(c3ccc(C4CC5CCC4C5)cc3)c3ccccc3-c3ccccc3-c3ccccc3)cc2-c2cc3ccccc3cc2C1(C)C ; CC1(C)c2ccccc2-c2ccc(N(c3ccc(C4CCCCC4)cc3)c3ccc4c(c3)-c3cc5ccccc5cc3C(C)(C)C4(C)C)cc21 ; CC1(C)c2ccccc2-c2ccc(N(c3ccc(C4CC5CCC4C5)cc3)c3ccc4c(c3)-c3cc5ccccc5cc3C(C)(C)C4(C)C)cc21]

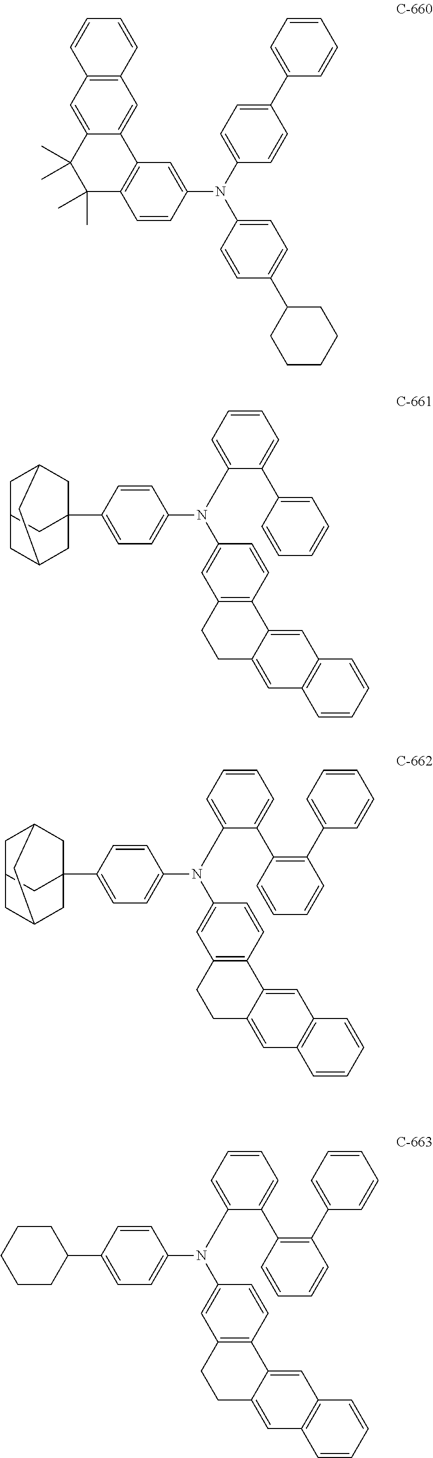 [SMILES: CC1(C)c2ccc(N(c3ccc(-c4ccccc4)cc3)c3ccc(C4CCCCC4)cc3)cc2-c2cc3ccccc3cc2C1(C)C ; c1ccc(-c2ccccc2N(c2ccc(C34CC5CC(CC(C5)C3)C4)cc2)c2ccc3c(c2)CCc2cc4ccccc4cc2-3)cc1 ; c1ccc(-c2ccccc2-c2ccccc2N(c2ccc(C34CC5CC(CC(C5)C3)C4)cc2)c2ccc3c(c2)CCc2cc4ccccc4cc2-3)cc1 ; c1ccc(-c2ccccc2-c2ccccc2N(c2ccc(C3CCCCC3)cc2)c2ccc3c(c2)CCc2cc4ccccc4cc2-3)cc1]

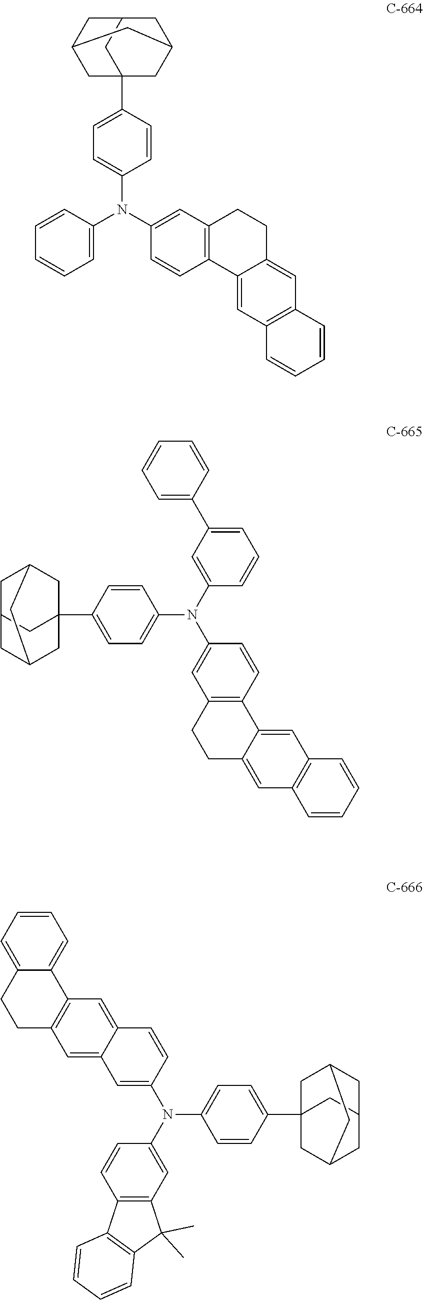 [SMILES: c1ccc(N(c2ccc(C34CC5CC(CC(C5)C3)C4)cc2)c2ccc3c(c2)CCc2cc4ccccc4cc2-3)cc1 ; c1ccc(-c2cccc(N(c3ccc(C45CC6CC(CC(C6)C4)C5)cc3)c3ccc4c(c3)CCc3cc5ccccc5cc3-4)c2)cc1 ; CC1(C)c2ccccc2-c2ccc(N(c3ccc(C45CC6CC(CC(C6)C4)C5)cc3)c3ccc4cc5c(cc4c3)CCc3ccccc3-5)cc21]

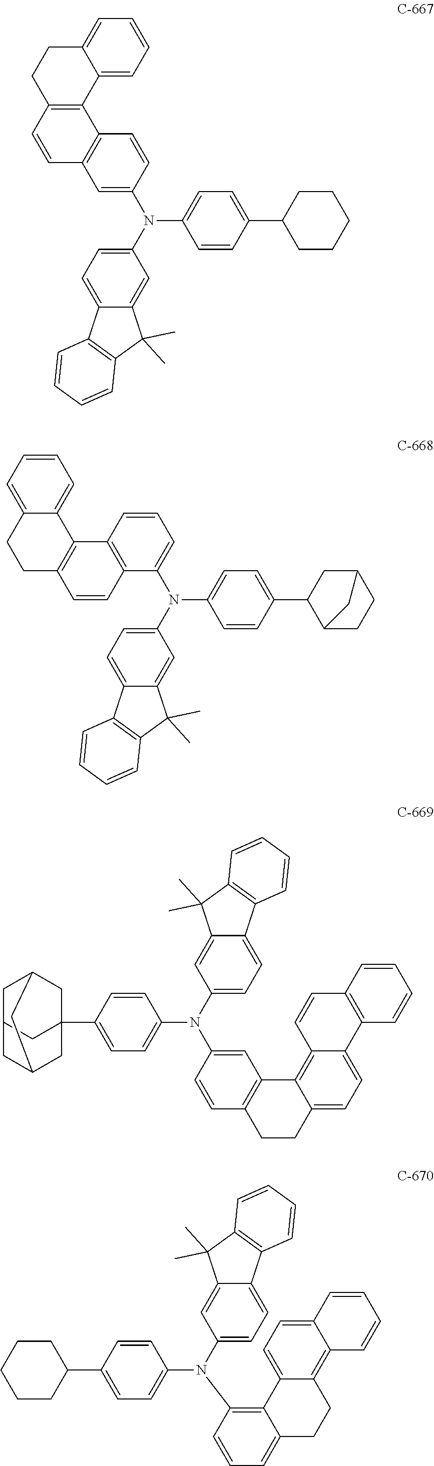 [SMILES: CC1(C)c2ccccc2-c2ccc(N(c3ccc(C4CCCCC4)cc3)c3ccc4c5c(ccc4c3)CCc3ccccc3-5)cc21 ; CC1(C)c2ccccc2-c2ccc(N(c3ccc(C4CC5CCC4C5)cc3)c3cccc4c5c(ccc34)CCc3ccccc3-5)cc21 ; CC1(C)c2ccccc2-c2ccc(N(c3ccc(C45CC6CC(CC(C6)C4)C5)cc3)c3ccc4c(c3)-c3c(ccc5c3ccc3ccccc35)CC4)cc21 ; CC1(C)c2ccccc2-c2ccc(N(c3ccc(C4CCCCC4)cc3)c3cccc4c3-c3ccc5ccccc5c3CC4)cc21]

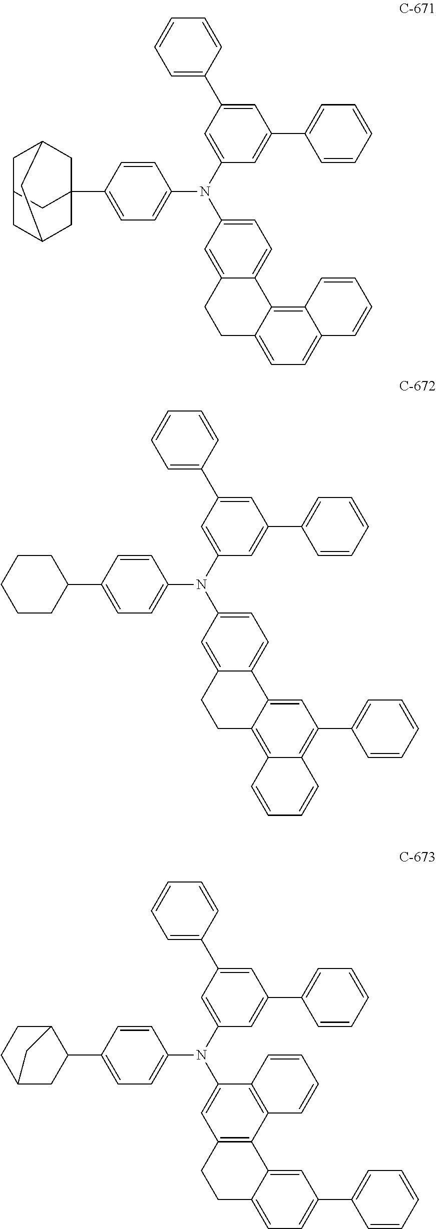 [SMILES: c1ccc(-c2cc(-c3ccccc3)cc(N(c3ccc(C45CC6CC(CC(C6)C4)C5)cc3)c3ccc4c(c3)CCc3ccc5ccccc5c3-4)c2)cc1 ; c1ccc(-c2cc(-c3ccccc3)cc(N(c3ccc(C4CCCCC4)cc3)c3ccc4c(c3)CCc3c-4cc(-c4ccccc4)c4ccccc34)c2)cc1 ; c1ccc(-c2cc(-c3ccccc3)cc(N(c3ccc(C4CC5CCC4C5)cc3)c3cc4c(c5ccccc35)-c3cc(-c5ccccc5)ccc3CC4)c2)cc1]

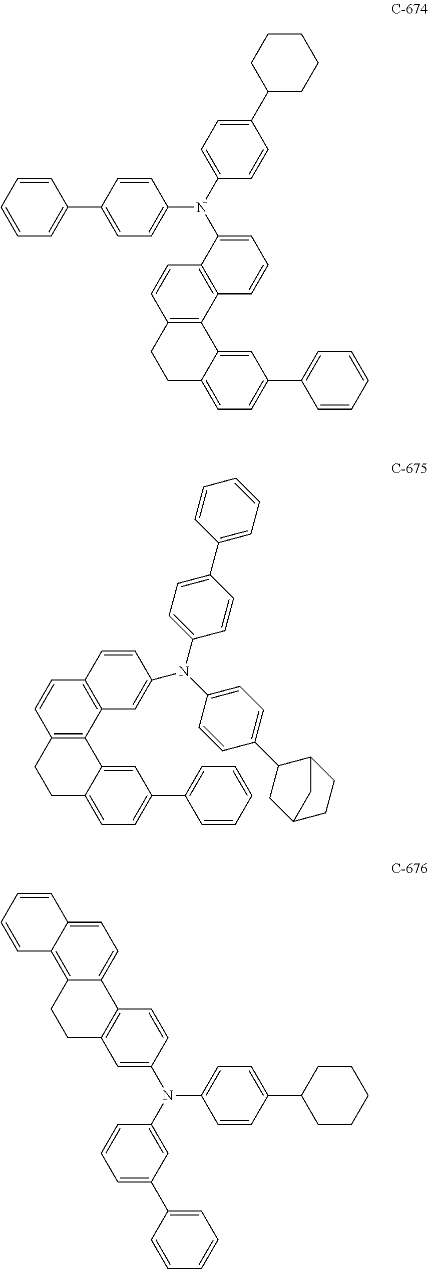 [SMILES: c1ccc(-c2ccc(N(c3ccc(C4CCCCC4)cc3)c3cccc4c5c(ccc34)CCc3ccc(-c4ccccc4)cc3-5)cc2)cc1 ; c1ccc(-c2ccc(N(c3ccc(C4CC5CCC4C5)cc3)c3ccc4ccc5c(c4c3)-c3cc(-c4ccccc4)ccc3CC5)cc2)cc1 ; c1ccc(-c2cccc(N(c3ccc(C4CCCCC4)cc3)c3ccc4c(c3)CCc3c-4ccc4ccccc34)c2)cc1]

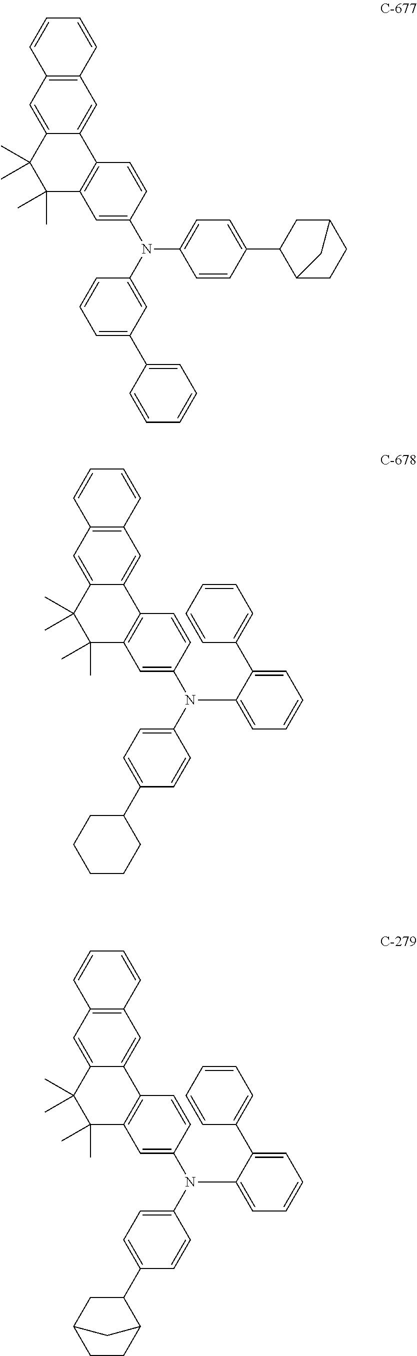 [SMILES: CC1(C)c2cc(N(c3ccc(C4CC5CCC4C5)cc3)c3cccc(-c4ccccc4)c3)ccc2-c2cc3ccccc3cc2C1(C)C ; CC1(C)c2cc(N(c3ccc(C4CCCCC4)cc3)c3ccccc3-c3ccccc3)ccc2-c2cc3ccccc3cc2C1(C)C ; CC1(C)c2cc(N(c3ccc(C4CC5CCC4C5)cc3)c3ccccc3-c3ccccc3)ccc2-c2cc3ccccc3cc2C1(C)C]

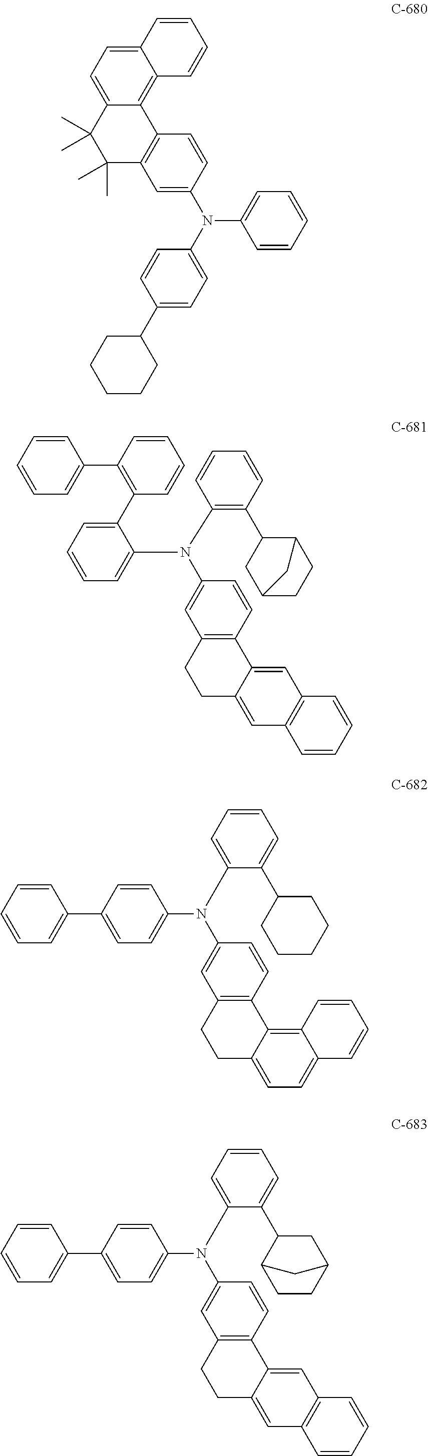

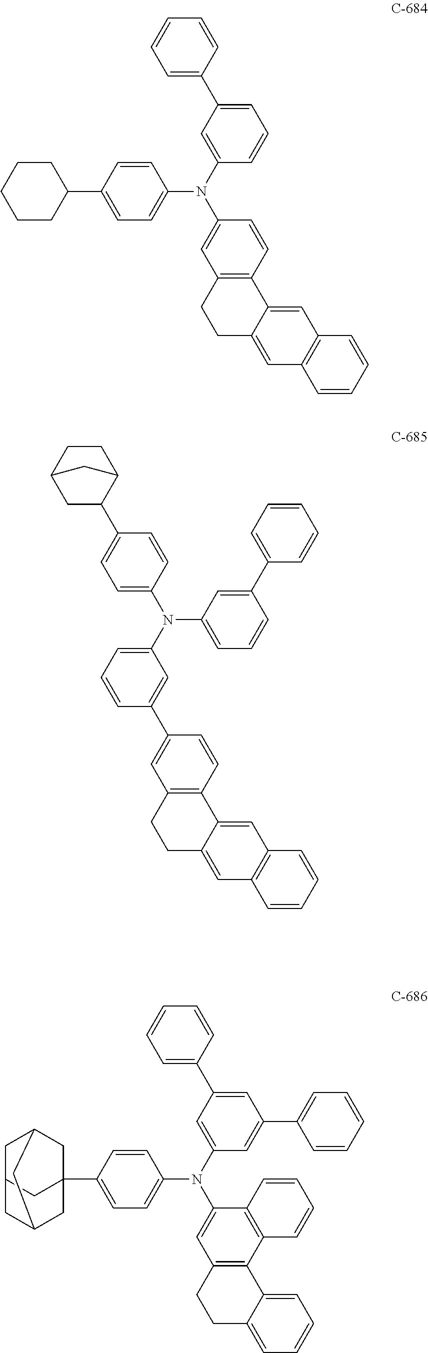 [SMILES: c1ccc(-c2cccc(N(c3ccc(C4CCCCC4)cc3)c3ccc4c(c3)CCc3cc5ccccc5cc3-4)c2)cc1 ; c1ccc(-c2cccc(N(c3ccc(C4CC5CCC4C5)cc3)c3cccc(-c4ccc5c(c4)CCc4cc6ccccc6cc4-5)c3)c2)cc1 ; c1ccc(-c2cc(-c3ccccc3)cc(N(c3ccc(C45CC6CC(CC(C6)C4)C5)cc3)c3cc4c(c5ccccc35)-c3ccccc3CC4)c2)cc1]

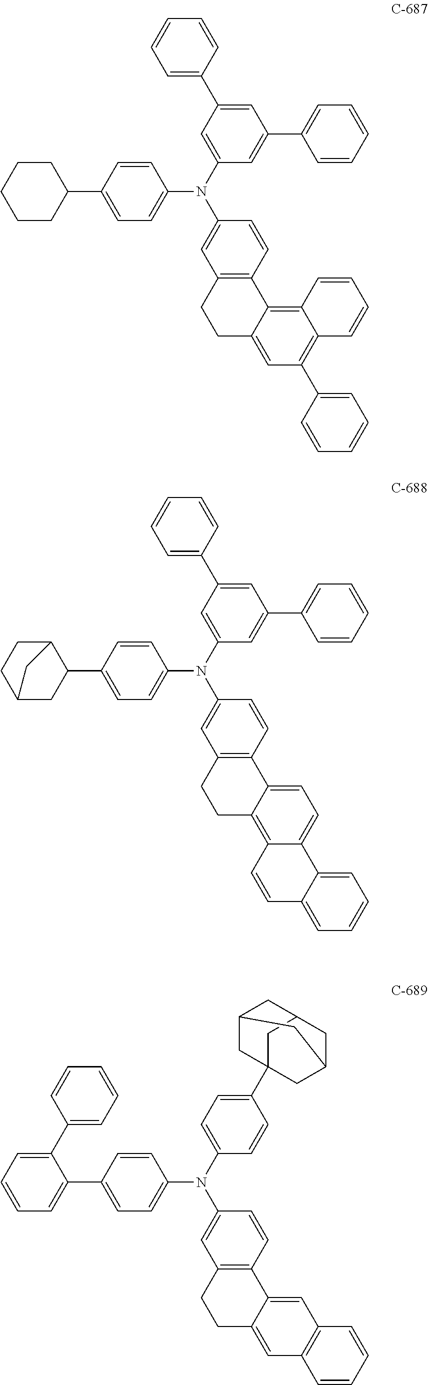 [SMILES: c1ccc(-c2cc(-c3ccccc3)cc(N(c3ccc(C4CCCCC4)cc3)c3ccc4c(c3)CCc3cc(-c5ccccc5)c5ccccc5c3-4)c2)cc1 ; c1ccc(-c2cc(-c3ccccc3)cc(N(c3ccc(C4CC5CCC4C5)cc3)c3ccc4c(c3)CCc3c-4ccc4c3ccc3ccccc34)c2)cc1 ; c1ccc(-c2ccccc2-c2ccc(N(c3ccc(C45CC6CC(CC(C6)C4)C5)cc3)c3ccc4c(c3)CCc3cc5ccccc5cc3-4)cc2)cc1]

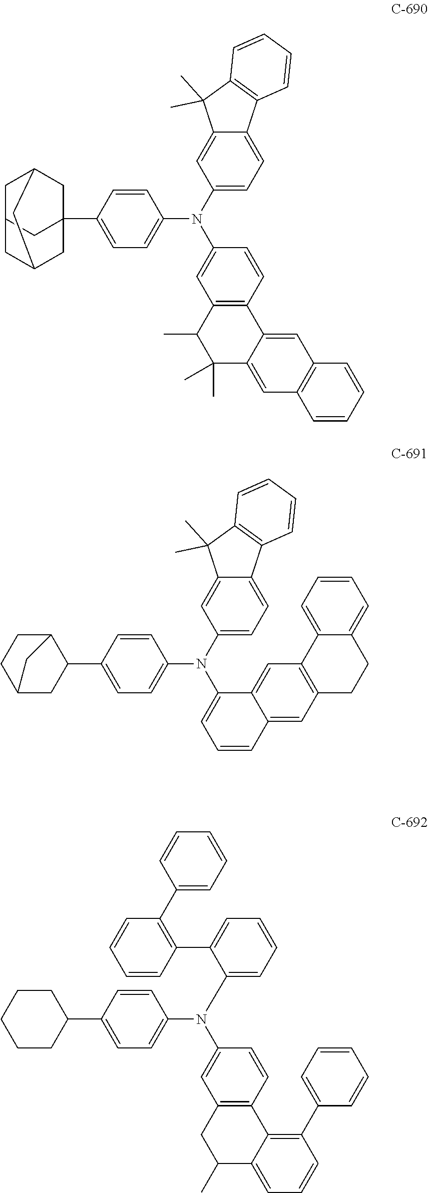 [SMILES: CC1c2cc(N(c3ccc(C45CC6CC(CC(C6)C4)C5)cc3)c3ccc4c(c3)C(C)(C)c3ccccc3-4)ccc2-c2cc3ccccc3cc2C1(C)C ; CC1(C)c2ccccc2-c2ccc(N(c3ccc(C4CC5CCC4C5)cc3)c3cccc4cc5c(cc34)-c3ccccc3CC5)cc21 ; CC1Cc2cc(N(c3ccc(C4CCCCC4)cc3)c3ccccc3-c3ccccc3-c3ccccc3)ccc2-c2c(-c3ccccc3)cccc21]

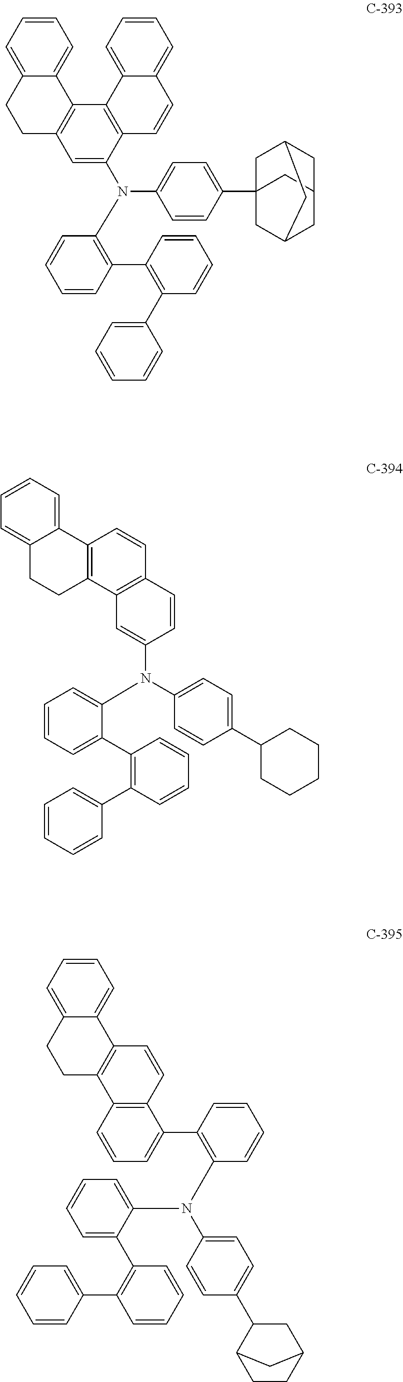 [SMILES: c1ccc(-c2ccccc2-c2ccccc2N(c2ccc(C34CC5CC(CC(C5)C3)C4)cc2)c2cc3c(c4c2ccc2ccccc24)-c2ccccc2CC3)cc1 ; c1ccc(-c2ccccc2-c2ccccc2N(c2ccc(C3CCCCC3)cc2)c2ccc3ccc4c(c3c2)CCc2ccccc2-4)cc1 ; c1ccc(-c2ccccc2-c2ccccc2N(c2ccc(C3CC4CCC3C4)cc2)c2ccccc2-c2cccc3c4c(ccc23)-c2ccccc2CC4)cc1]

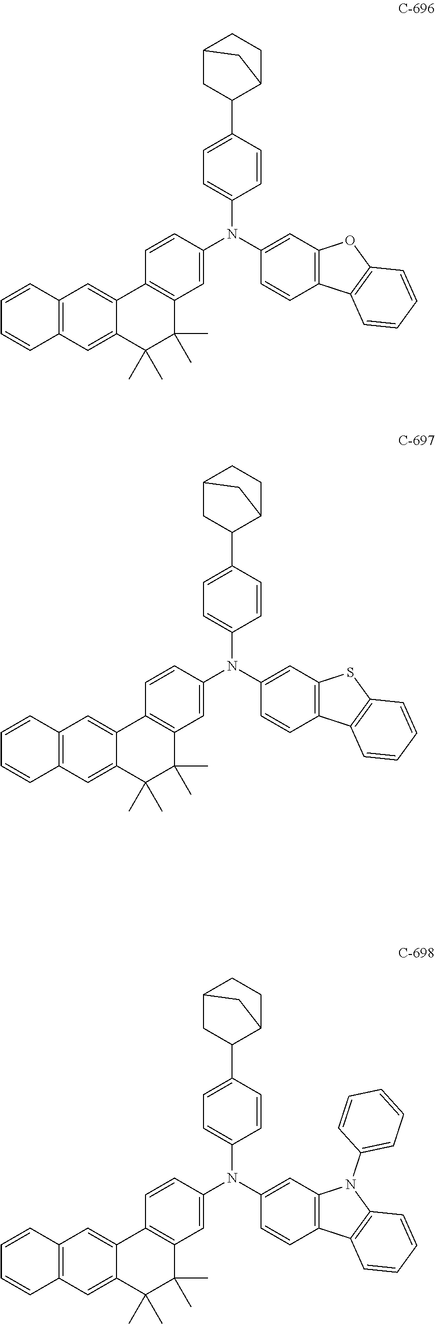 [SMILES: CC1(C)c2cc(N(c3ccc(C4CC5CCC4C5)cc3)c3ccc4c(c3)oc3ccccc34)ccc2-c2cc3ccccc3cc2C1(C)C ; CC1(C)c2cc(N(c3ccc(C4CC5CCC4C5)cc3)c3ccc4c(c3)sc3ccccc34)ccc2-c2cc3ccccc3cc2C1(C)C ; CC1(C)c2cc(N(c3ccc(C4CC5CCC4C5)cc3)c3ccc4c5ccccc5n(-c5ccccc5)c4c3)ccc2-c2cc3ccccc3cc2C1(C)C]

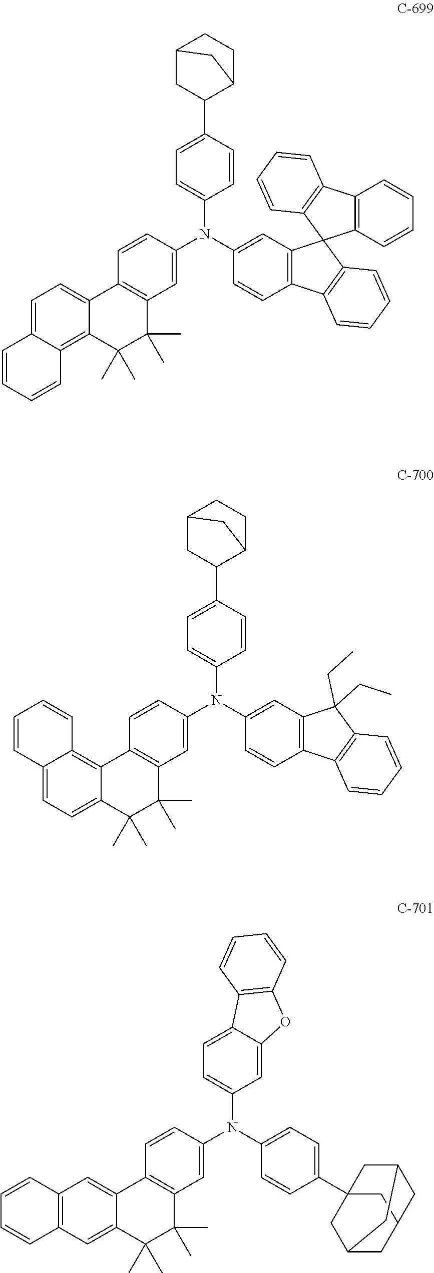 [SMILES: CC1(C)c2cc(N(c3ccc(C4CC5CCC4C5)cc3)c3ccc4c(c3)C3(c5ccccc5-c5ccccc53)c3ccccc3-4)ccc2-c2ccc3ccccc3c2C1(C)C ; CCC1(CC)c2ccccc2-c2ccc(N(c3ccc(C4CC5CCC4C5)cc3)c3ccc4c(c3)C(C)(C)C(C)(C)c3ccc5ccccc5c3-4)cc21 ; CC1(C)c2cc(N(c3ccc(C45CC6CC(CC(C6)C4)C5)cc3)c3ccc4c(c3)oc3ccccc34)ccc2-c2cc3ccccc3cc2C1(C)C]

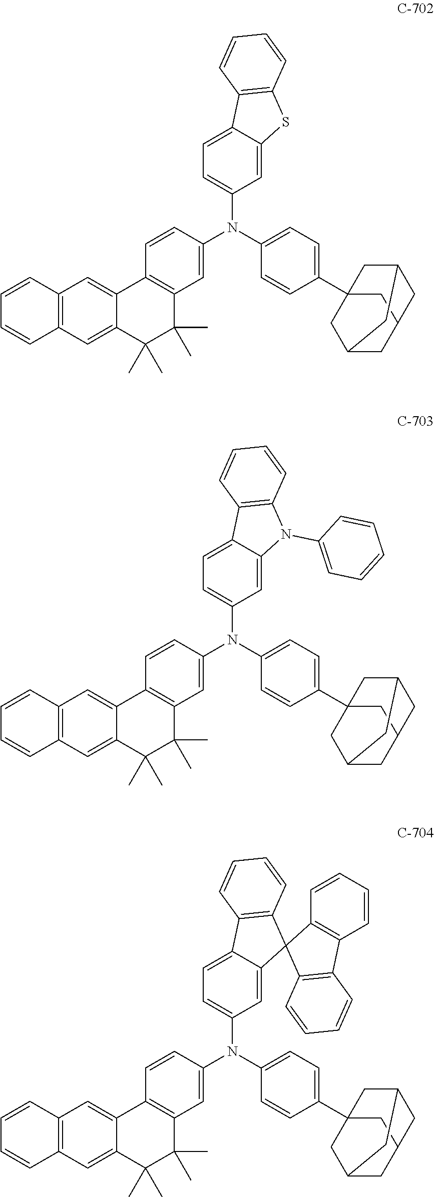 [SMILES: CC1(C)c2cc(N(c3ccc(C45CC6CC(CC(C6)C4)C5)cc3)c3ccc4c(c3)sc3ccccc34)ccc2-c2cc3ccccc3cc2C1(C)C ; CC1(C)c2cc(N(c3ccc(C45CC6CC(CC(C6)C4)C5)cc3)c3ccc4c5ccccc5n(-c5ccccc5)c4c3)ccc2-c2cc3ccccc3cc2C1(C)C ; CC1(C)c2cc(N(c3ccc(C45CC6CC(CC(C6)C4)C5)cc3)c3ccc4c(c3)C3(c5ccccc5-c5ccccc53)c3ccccc3-4)ccc2-c2cc3ccccc3cc2C1(C)C]

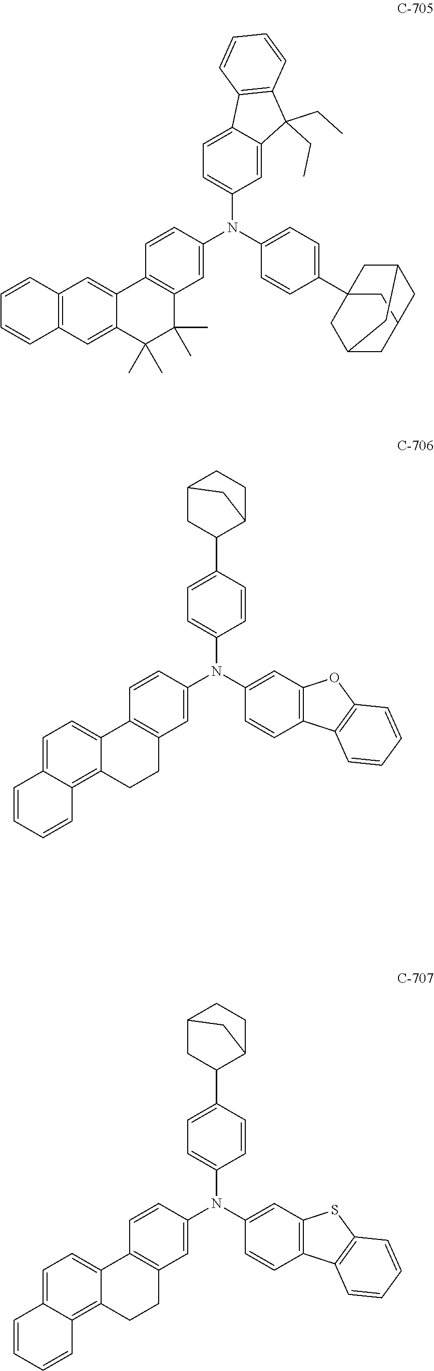 [SMILES: CCC1(CC)c2ccccc2-c2ccc(N(c3ccc(C45CC6CC(CC(C6)C4)C5)cc3)c3ccc4c(c3)C(C)(C)C(C)(C)c3cc5ccccc5cc3-4)cc21 ; c1ccc2c3c(ccc2c1)-c1ccc(N(c2ccc(C4CC5CCC4C5)cc2)c2ccc4c(c2)oc2ccccc24)cc1CC3 ; c1ccc2c3c(ccc2c1)-c1ccc(N(c2ccc(C4CC5CCC4C5)cc2)c2ccc4c(c2)sc2ccccc24)cc1CC3]

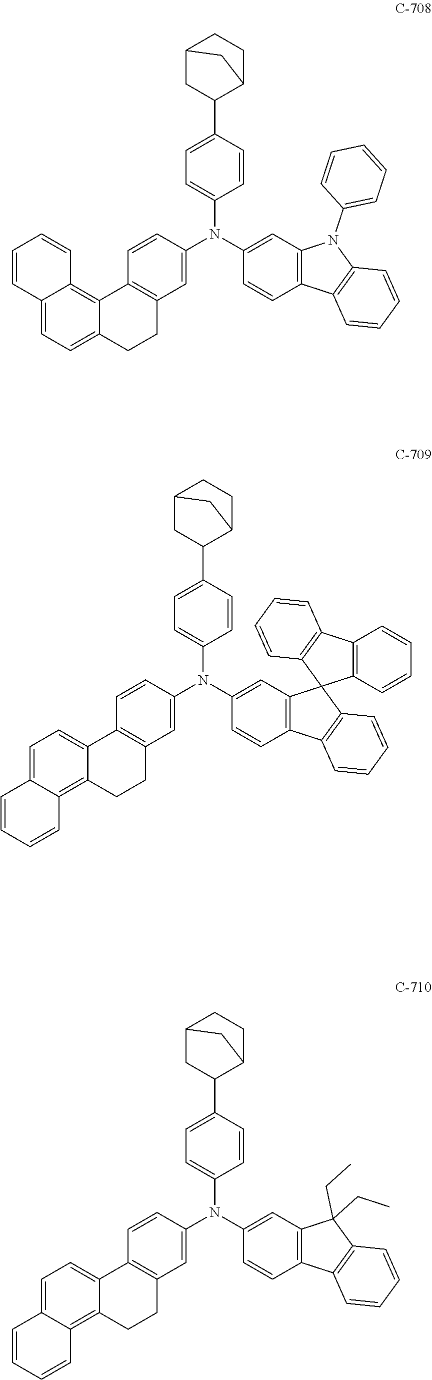 [SMILES: c1ccc(-n2c3ccccc3c3ccc(N(c4ccc(C5CC6CCC5C6)cc4)c4ccc5c(c4)CCc4ccc6ccccc6c4-5)cc32)cc1 ; c1ccc2c(c1)-c1ccccc1C21c2ccccc2-c2ccc(N(c3ccc(C4CC5CCC4C5)cc3)c3ccc4c(c3)CCc3c-4ccc4ccccc34)cc21 ; CCC1(CC)c2ccccc2-c2ccc(N(c3ccc(C4CC5CCC4C5)cc3)c3ccc4c(c3)CCc3c-4ccc4ccccc34)cc21]

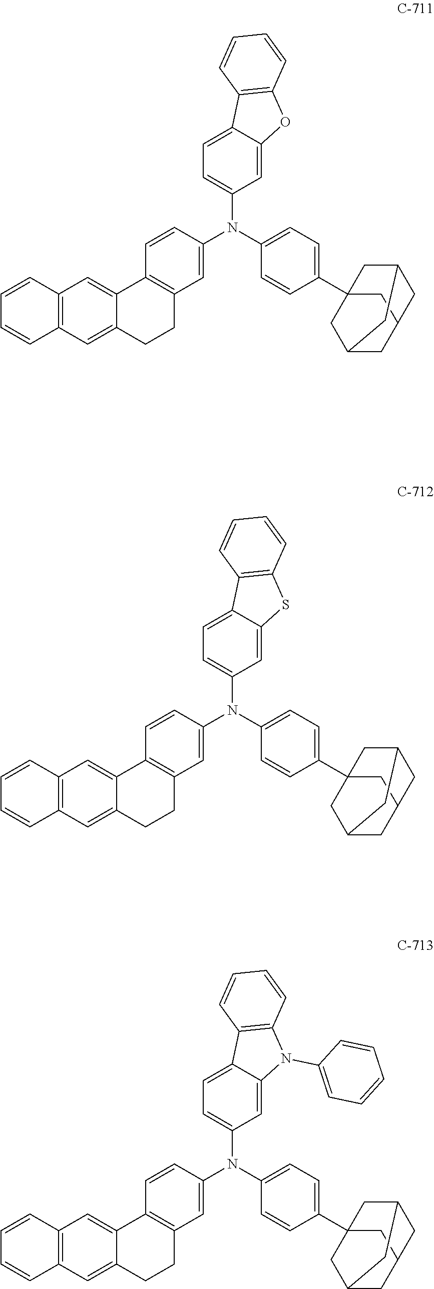 [SMILES: c1ccc2cc3c(cc2c1)CCc1cc(N(c2ccc(C45CC6CC(CC(C6)C4)C5)cc2)c2ccc4c(c2)oc2ccccc24)ccc1-3 ; c1ccc2cc3c(cc2c1)CCc1cc(N(c2ccc(C45CC6CC(CC(C6)C4)C5)cc2)c2ccc4c(c2)sc2ccccc24)ccc1-3 ; c1ccc(-n2c3ccccc3c3ccc(N(c4ccc(C56CC7CC(CC(C7)C5)C6)cc4)c4ccc5c(c4)CCc4cc6ccccc6cc4-5)cc32)cc1]

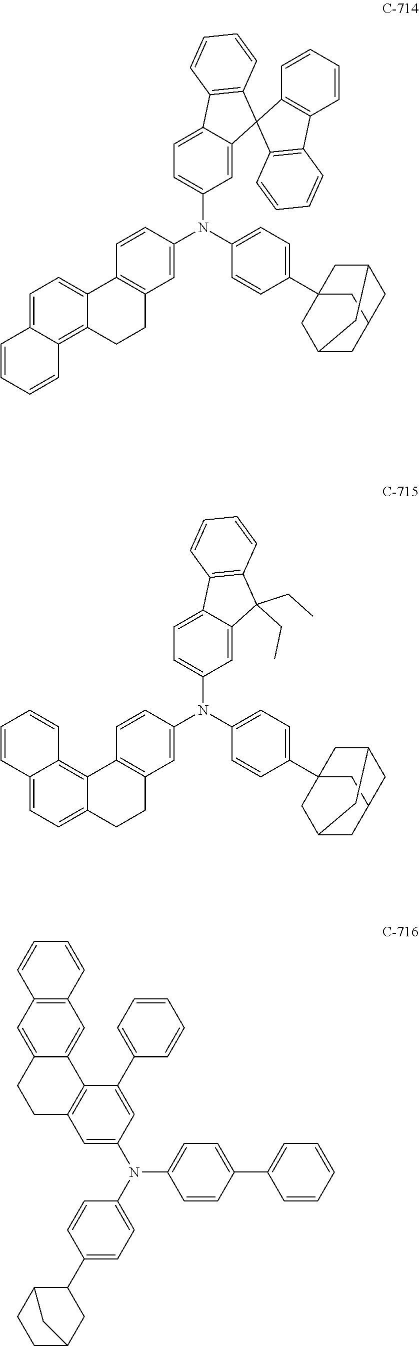 [SMILES: c1ccc2c(c1)-c1ccccc1C21c2ccccc2-c2ccc(N(c3ccc(C45CC6CC(CC(C6)C4)C5)cc3)c3ccc4c(c3)CCc3c-4ccc4ccccc34)cc21 ; CCC1(CC)c2ccccc2-c2ccc(N(c3ccc(C45CC6CC(CC(C6)C4)C5)cc3)c3ccc4c(c3)CCc3ccc5ccccc5c3-4)cc21 ; c1ccc(-c2ccc(N(c3ccc(C4CC5CCC4C5)cc3)c3cc4c(c(-c5ccccc5)c3)-c3cc5ccccc5cc3CC4)cc2)cc1]

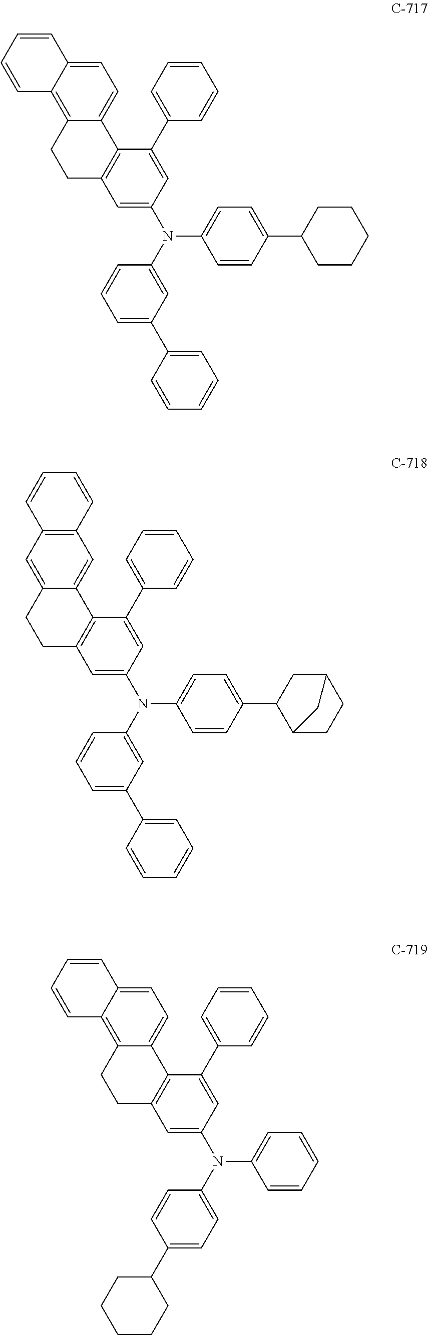 [SMILES: c1ccc(-c2cccc(N(c3ccc(C4CCCCC4)cc3)c3cc4c(c(-c5ccccc5)c3)-c3ccc5ccccc5c3CC4)c2)cc1 ; c1ccc(-c2cccc(N(c3ccc(C4CC5CCC4C5)cc3)c3cc4c(c(-c5ccccc5)c3)-c3cc5ccccc5cc3CC4)c2)cc1 ; c1ccc(-c2cc(N(c3ccccc3)c3ccc(C4CCCCC4)cc3)cc3c2-c2ccc4ccccc4c2CC3)cc1]

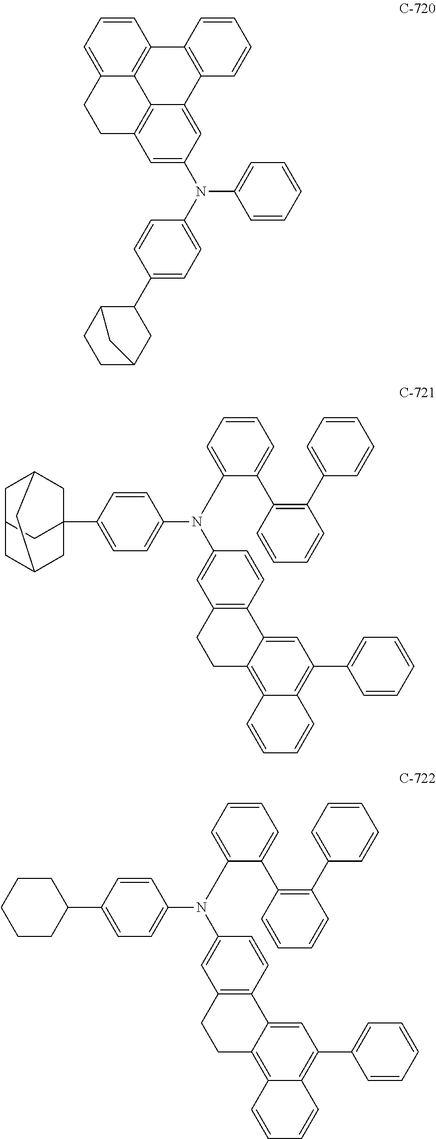 [SMILES: c1ccc(N(c2ccc(C3CC4CCC3C4)cc2)c2cc3c4c(c2)c2ccccc2c2cccc(c24)CC3)cc1 ; c1ccc(-c2ccccc2-c2ccccc2N(c2ccc(C34CC5CC(CC(C5)C3)C4)cc2)c2ccc3c(c2)CCc2c-3cc(-c3ccccc3)c3ccccc23)cc1 ; c1ccc(-c2ccccc2-c2ccccc2N(c2ccc(C3CCCCC3)cc2)c2ccc3c(c2)CCc2c-3cc(-c3ccccc3)c3ccccc23)cc1]

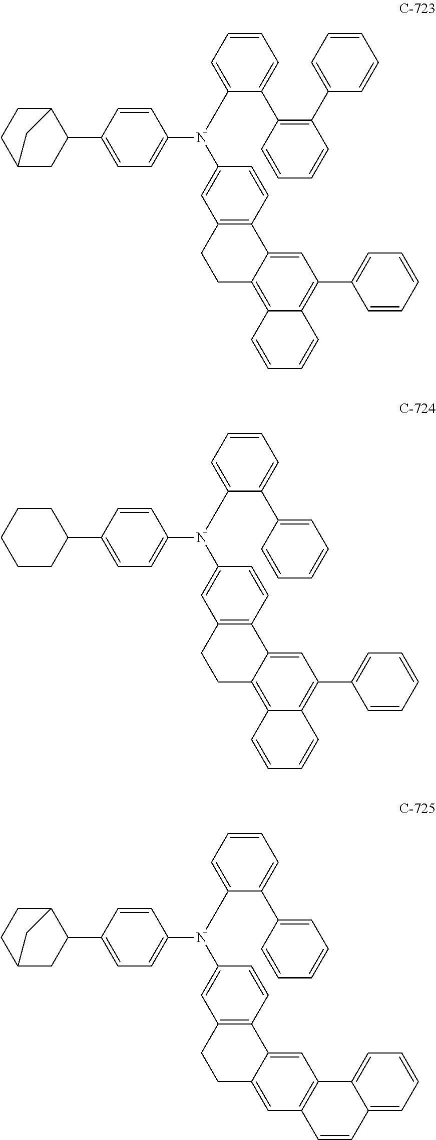 [SMILES: c1ccc(-c2ccccc2-c2ccccc2N(c2ccc(C3CC4CCC3C4)cc2)c2ccc3c(c2)CCc2c-3cc(-c3ccccc3)c3ccccc23)cc1 ; c1ccc(-c2ccccc2N(c2ccc(C3CCCCC3)cc2)c2ccc3c(c2)CCc2c-3cc(-c3ccccc3)c3ccccc23)cc1 ; c1ccc(-c2ccccc2N(c2ccc(C3CC4CCC3C4)cc2)c2ccc3c(c2)CCc2cc4ccc5ccccc5c4cc2-3)cc1]

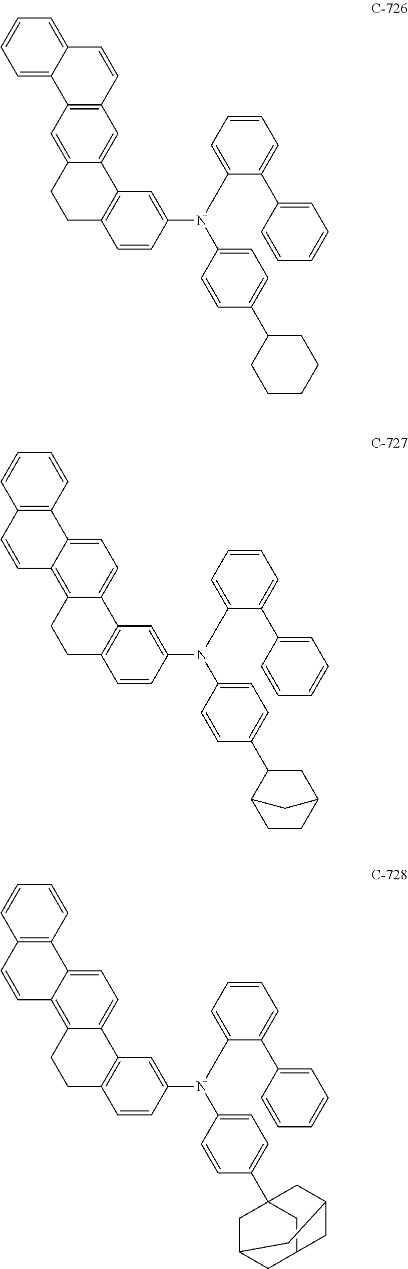 [SMILES: c1ccc(-c2ccccc2N(c2ccc(C3CCCCC3)cc2)c2ccc3c(c2)-c2cc4ccc5ccccc5c4cc2CC3)cc1 ; c1ccc(-c2ccccc2N(c2ccc(C3CC4CCC3C4)cc2)c2ccc3c(c2)-c2ccc4c(ccc5ccccc54)c2CC3)cc1 ; c1ccc(-c2ccccc2N(c2ccc(C34CC5CC(CC(C5)C3)C4)cc2)c2ccc3c(c2)-c2ccc4c(ccc5ccccc54)c2CC3)cc1]

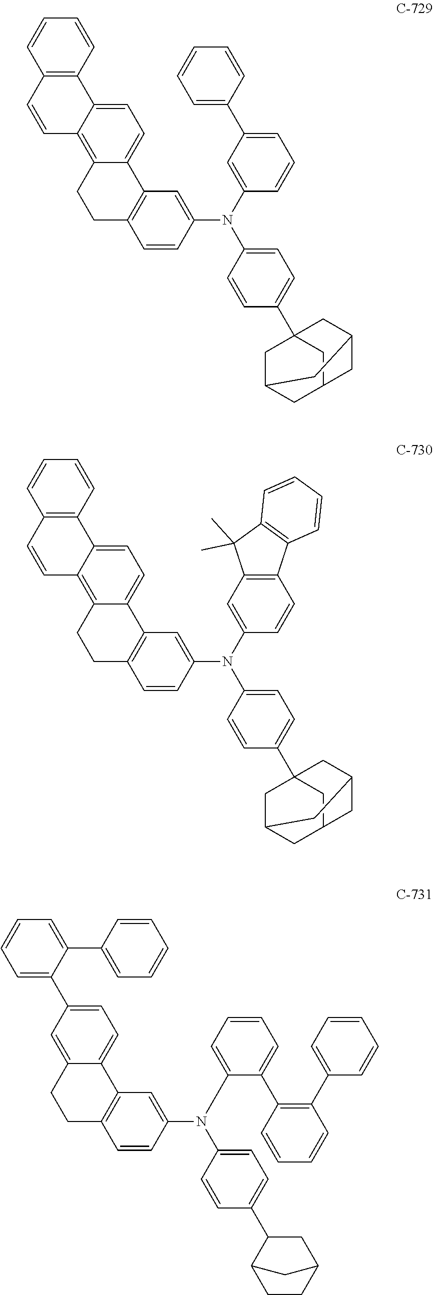 [SMILES: c1ccc(-c2cccc(N(c3ccc(C45CC6CC(CC(C6)C4)C5)cc3)c3ccc4c(c3)-c3ccc5c(ccc6ccccc65)c3CC4)c2)cc1 ; CC1(C)c2ccccc2-c2ccc(N(c3ccc(C45CC6CC(CC(C6)C4)C5)cc3)c3ccc4c(c3)-c3ccc5c(ccc6ccccc65)c3CC4)cc21 ; c1ccc(-c2ccccc2-c2ccc3c(c2)CCc2ccc(N(c4ccc(C5CC6CCC5C6)cc4)c4ccccc4-c4ccccc4-c4ccccc4)cc2-3)cc1]

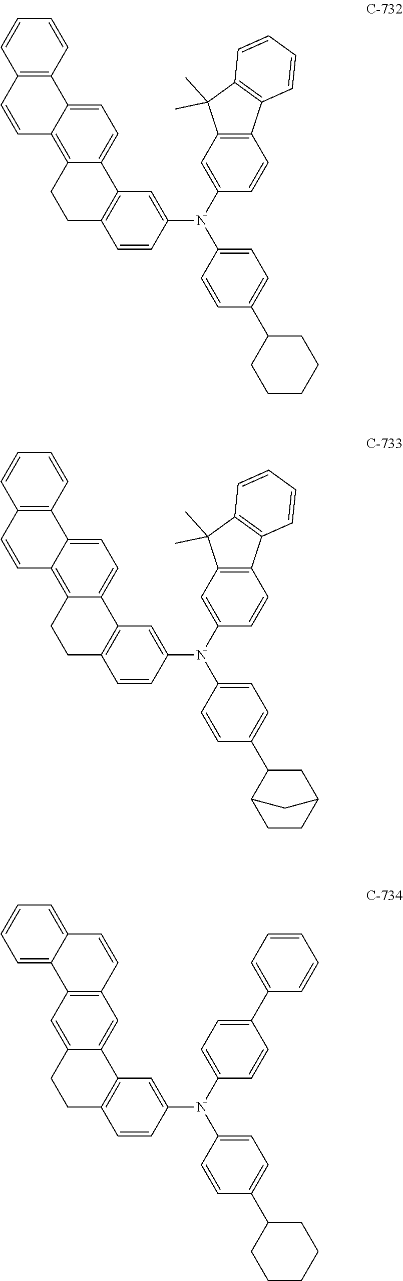 [SMILES: CC1(C)c2ccccc2-c2ccc(N(c3ccc(C4CCCCC4)cc3)c3ccc4c(c3)-c3ccc5c(ccc6ccccc65)c3CC4)cc21 ; CC1(C)c2ccccc2-c2ccc(N(c3ccc(C4CC5CCC4C5)cc3)c3ccc4c(c3)-c3ccc5c(ccc6ccccc65)c3CC4)cc21 ; c1ccc(-c2ccc(N(c3ccc(C4CCCCC4)cc3)c3ccc4c(c3)-c3cc5ccc6ccccc6c5cc3CC4)cc2)cc1]

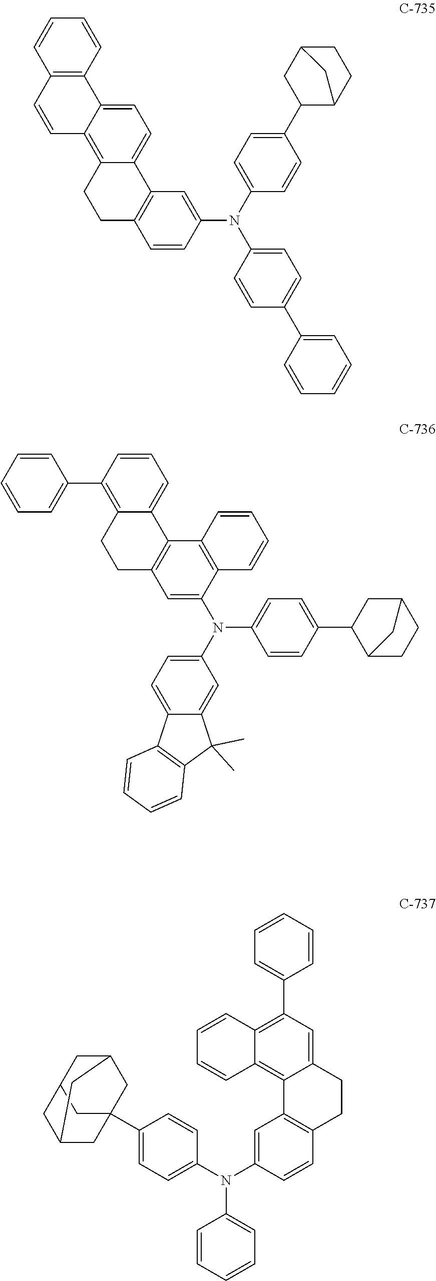 [SMILES: c1ccc(-c2ccc(N(c3ccc(C4CC5CCC4C5)cc3)c3ccc4c(c3)-c3ccc5c(ccc6ccccc65)c3CC4)cc2)cc1 ; CC1(C)c2ccccc2-c2ccc(N(c3ccc(C4CC5CCC4C5)cc3)c3cc4c(c5ccccc35)-c3cccc(-c5ccccc5)c3CC4)cc21 ; c1ccc(-c2cc3c(c4ccccc24)-c2cc(N(c4ccccc4)c4ccc(C56CC7CC(CC(C7)C5)C6)cc4)ccc2CC3)cc1]

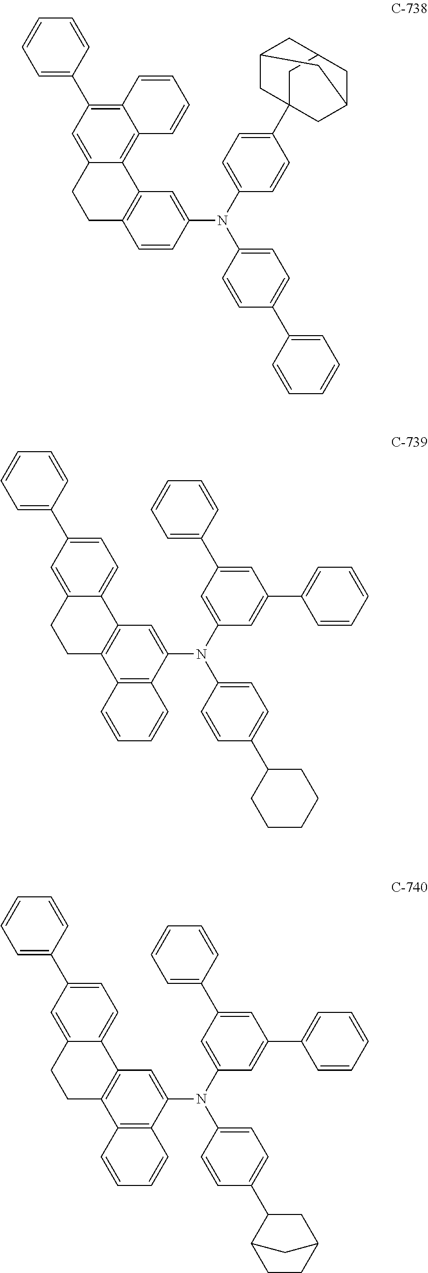 [SMILES: c1ccc(-c2ccc(N(c3ccc(C45CC6CC(CC(C6)C4)C5)cc3)c3ccc4c(c3)-c3c(cc(-c5ccccc5)c5ccccc35)CC4)cc2)cc1 ; c1ccc(-c2cc(-c3ccccc3)cc(N(c3ccc(C4CCCCC4)cc3)c3cc4c(c5ccccc35)CCc3cc(-c5ccccc5)ccc3-4)c2)cc1 ; c1ccc(-c2cc(-c3ccccc3)cc(N(c3ccc(C4CC5CCC4C5)cc3)c3cc4c(c5ccccc35)CCc3cc(-c5ccccc5)ccc3-4)c2)cc1]

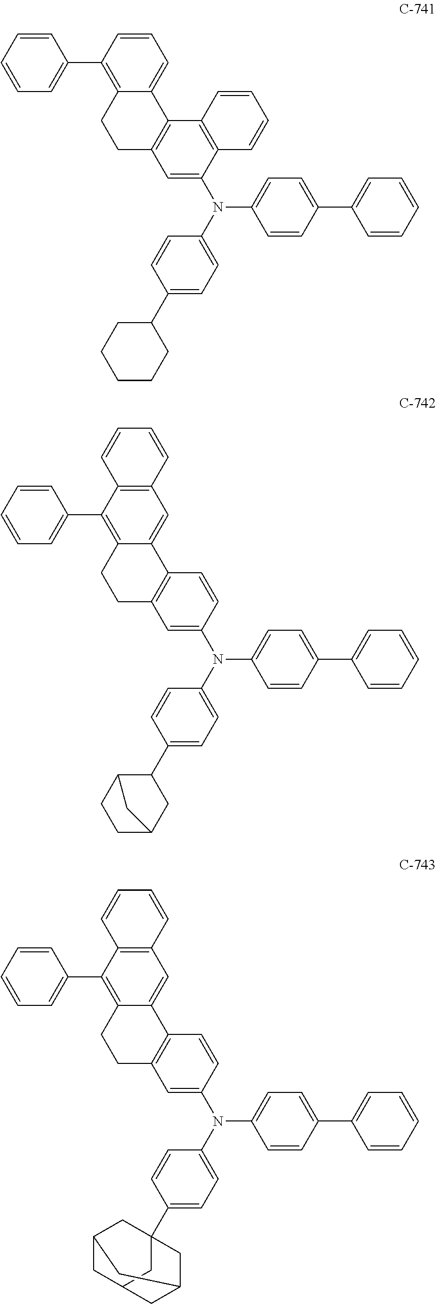 [SMILES: c1ccc(-c2ccc(N(c3ccc(C4CCCCC4)cc3)c3cc4c(c5ccccc35)-c3cccc(-c5ccccc5)c3CC4)cc2)cc1 ; c1ccc(-c2ccc(N(c3ccc(C4CC5CCC4C5)cc3)c3ccc4c(c3)CCc3c-4cc4ccccc4c3-c3ccccc3)cc2)cc1 ; c1ccc(-c2ccc(N(c3ccc(C45CC6CC(CC(C6)C4)C5)cc3)c3ccc4c(c3)CCc3c-4cc4ccccc4c3-c3ccccc3)cc2)cc1]

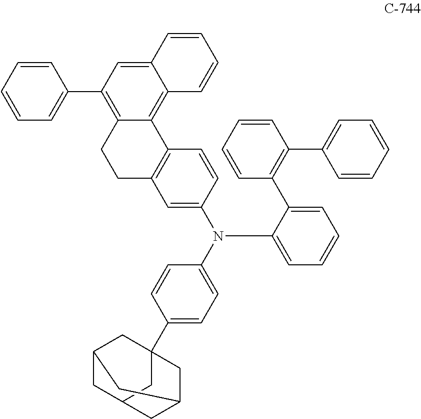 [SMILES: c1ccc(-c2ccccc2-c2ccccc2N(c2ccc(C34CC5CC(CC(C5)C3)C4)cc2)c2ccc3c(c2)CCc2c(-c4ccccc4)cc4ccccc4c2-3)cc1]

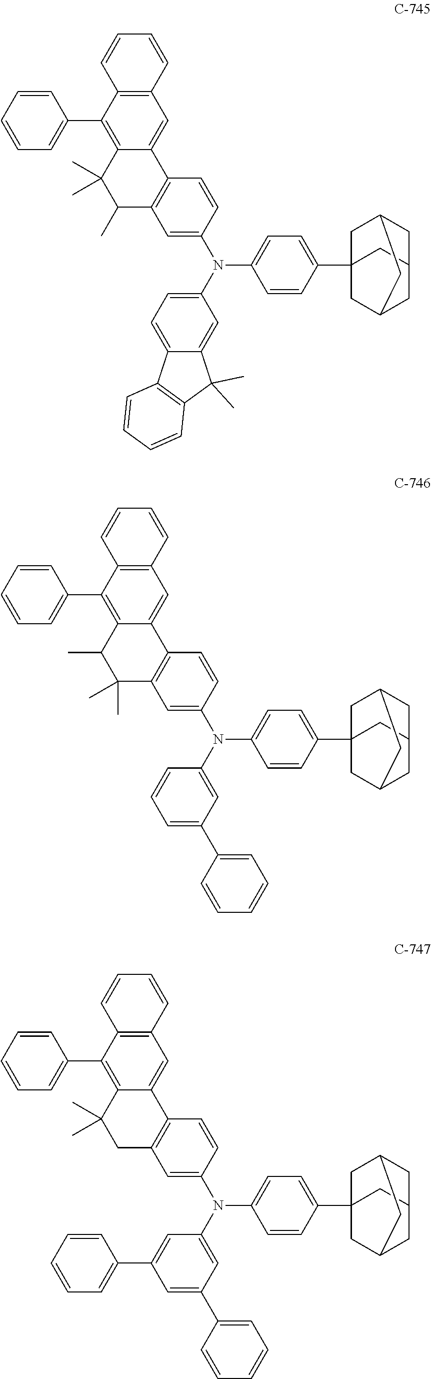 [SMILES: CC1c2cc(N(c3ccc(C45CC6CC(CC(C6)C4)C5)cc3)c3ccc4c(c3)C(C)(C)c3ccccc3-4)ccc2-c2cc3ccccc3c(-c3ccccc3)c2C1(C)C ; CC1c2c(cc3ccccc3c2-c2ccccc2)-c2ccc(N(c3ccc(C45CC6CC(CC(C6)C4)C5)cc3)c3cccc(-c4ccccc4)c3)cc2C1(C)C ; CC1(C)Cc2cc(N(c3ccc(C45CC6CC(CC(C6)C4)C5)cc3)c3cc(-c4ccccc4)cc(-c4ccccc4)c3)ccc2-c2cc3ccccc3c(-c3ccccc3)c21]

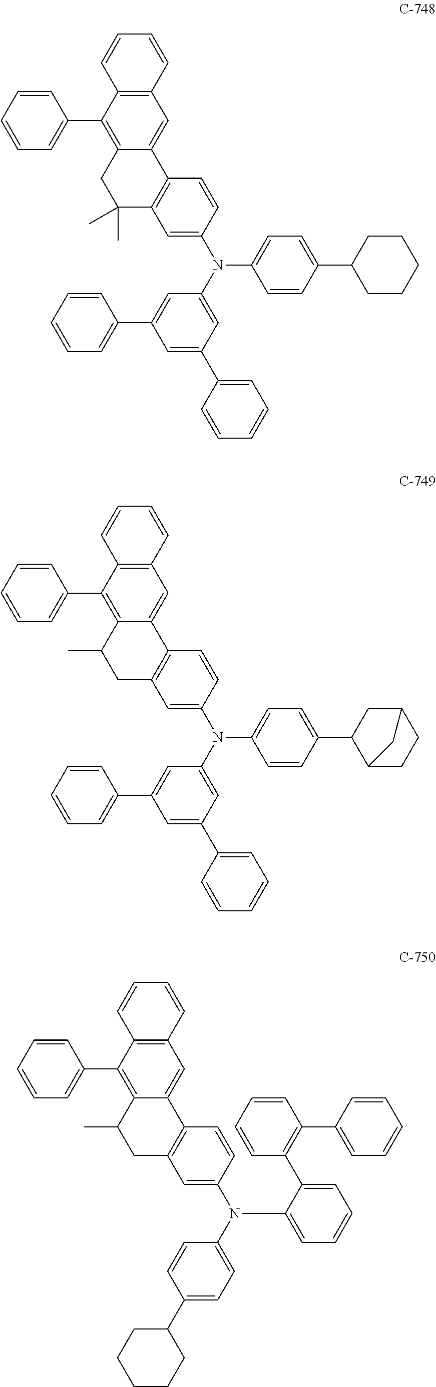 [SMILES: CC1(C)Cc2c(cc3ccccc3c2-c2ccccc2)-c2ccc(N(c3ccc(C4CCCCC4)cc3)c3cc(-c4ccccc4)cc(-c4ccccc4)c3)cc21 ; CC1Cc2cc(N(c3ccc(C4CC5CCC4C5)cc3)c3cc(-c4ccccc4)cc(-c4ccccc4)c3)ccc2-c2cc3ccccc3c(-c3ccccc3)c21 ; CC1Cc2cc(N(c3ccc(C4CCCCC4)cc3)c3ccccc3-c3ccccc3-c3ccccc3)ccc2-c2cc3ccccc3c(-c3ccccc3)c21]

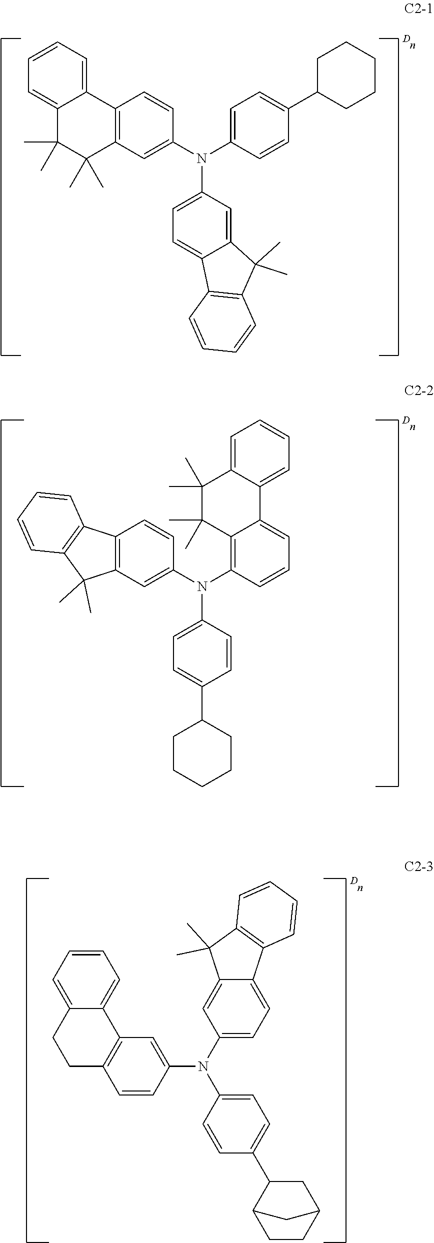

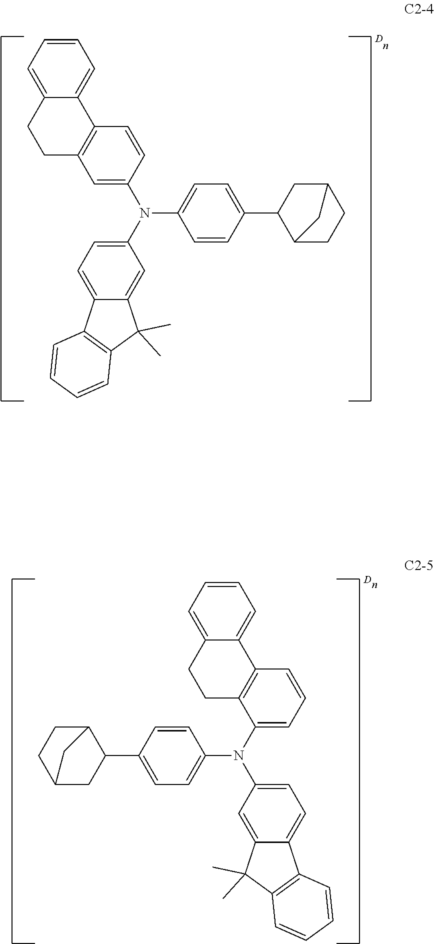

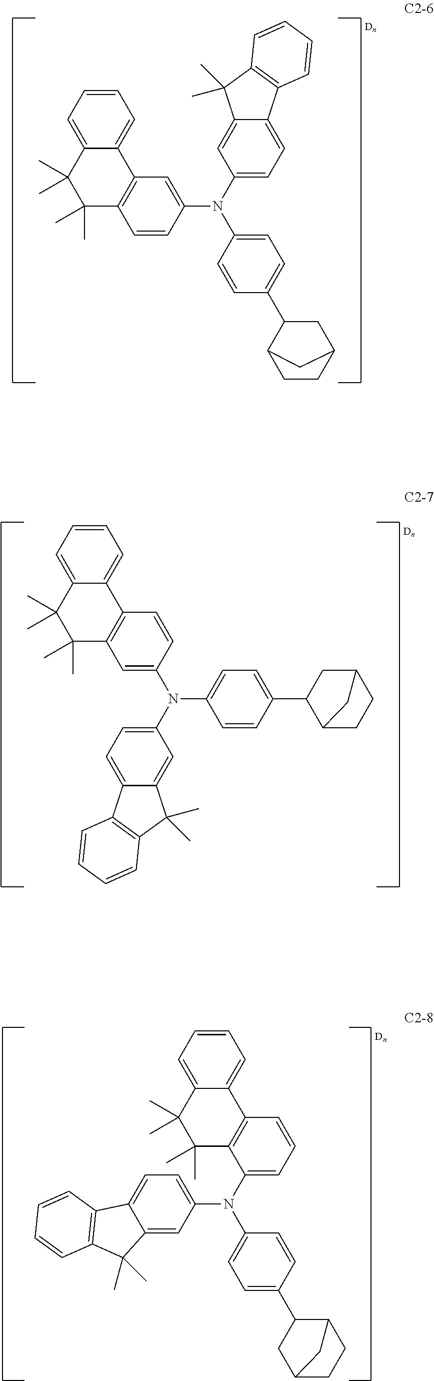

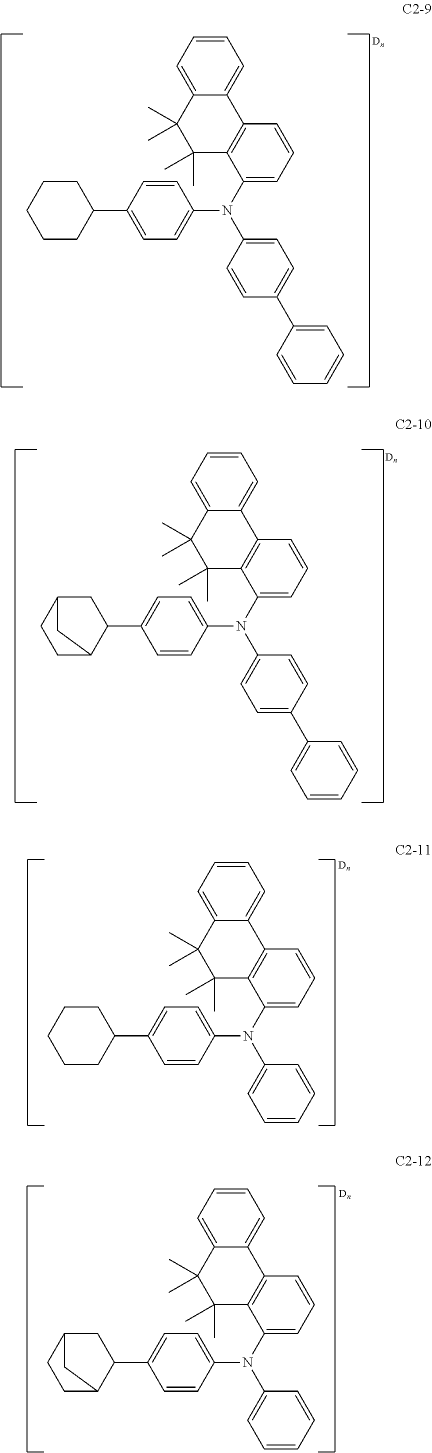

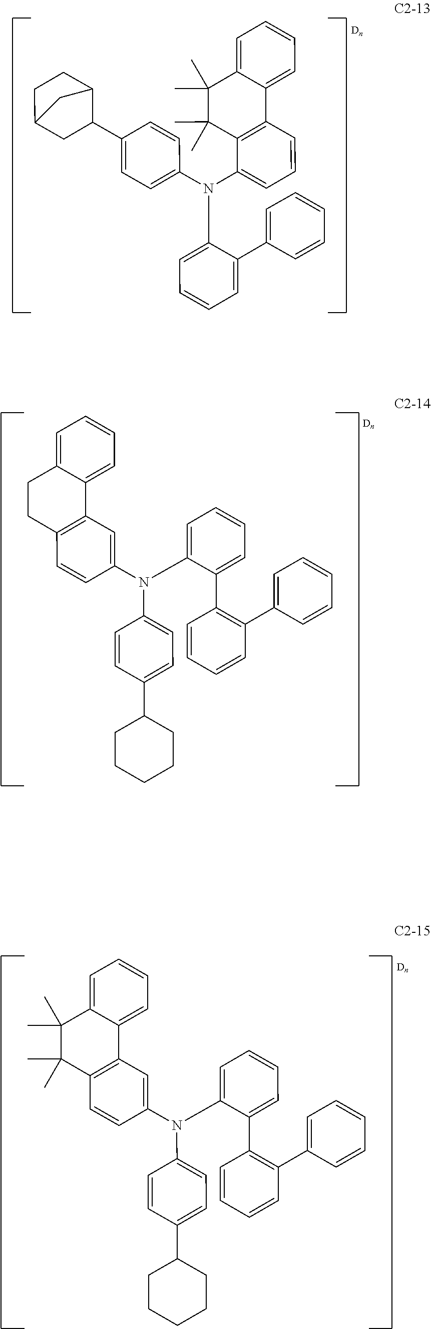

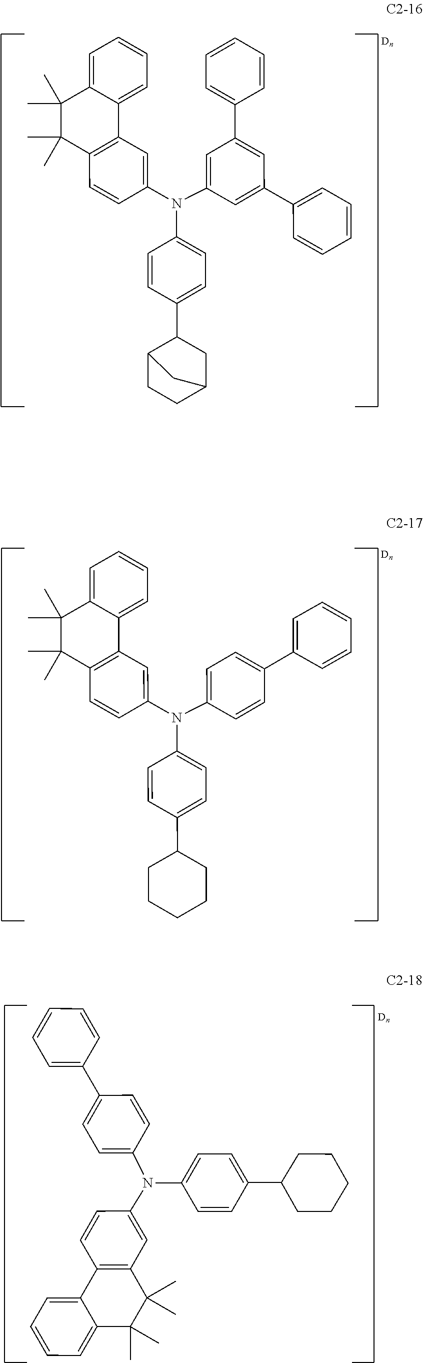

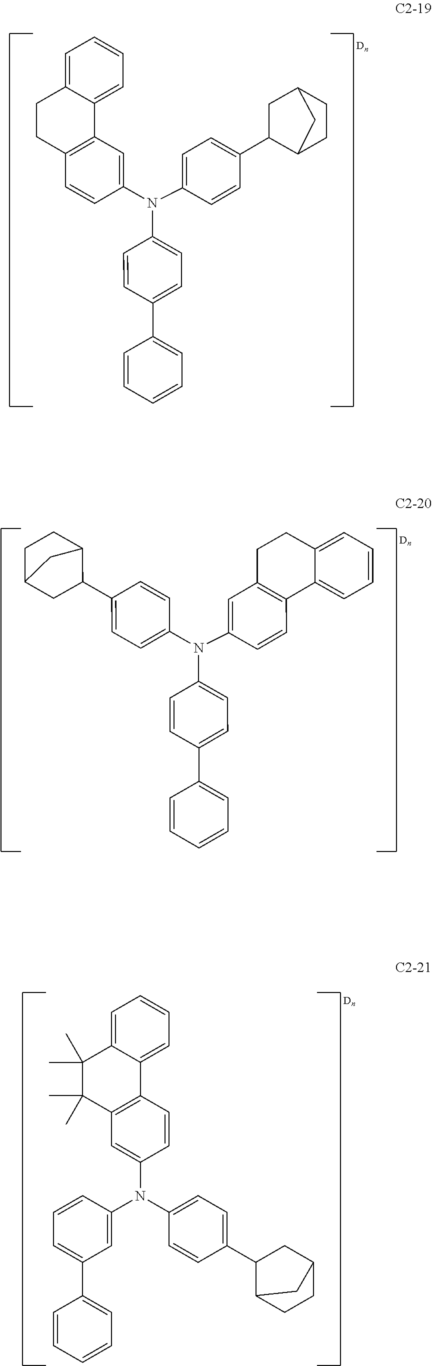

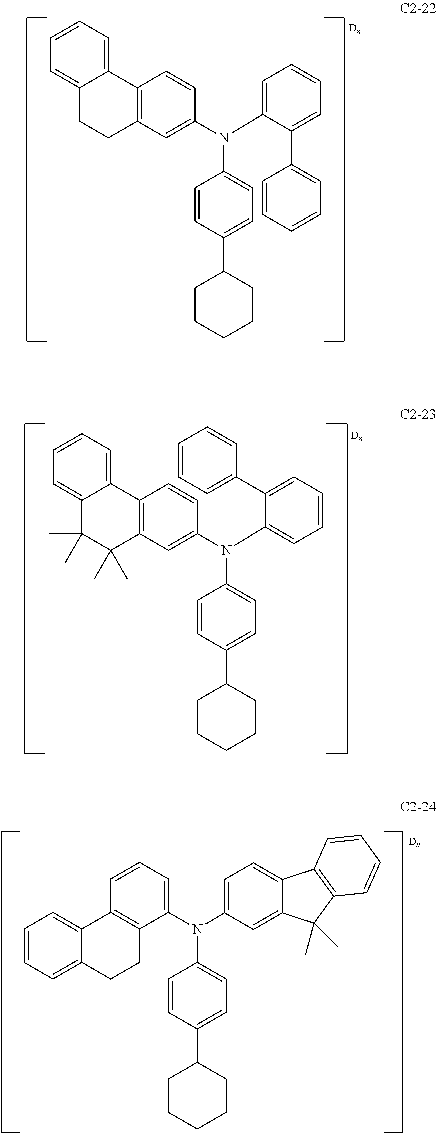

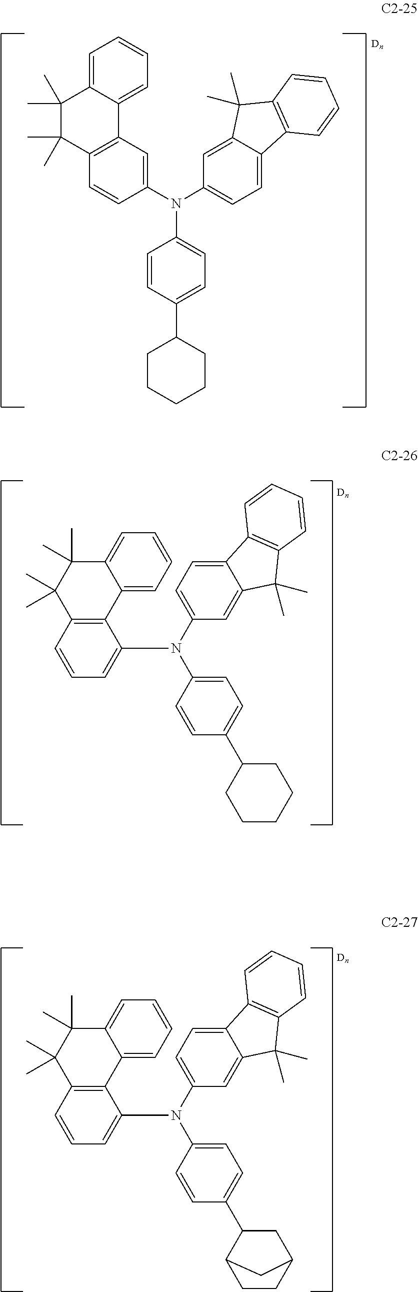

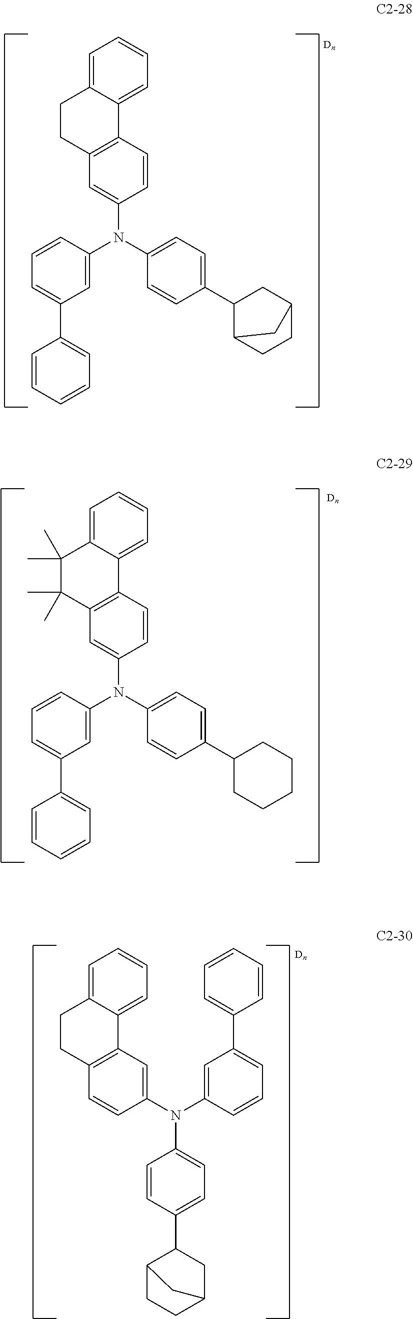

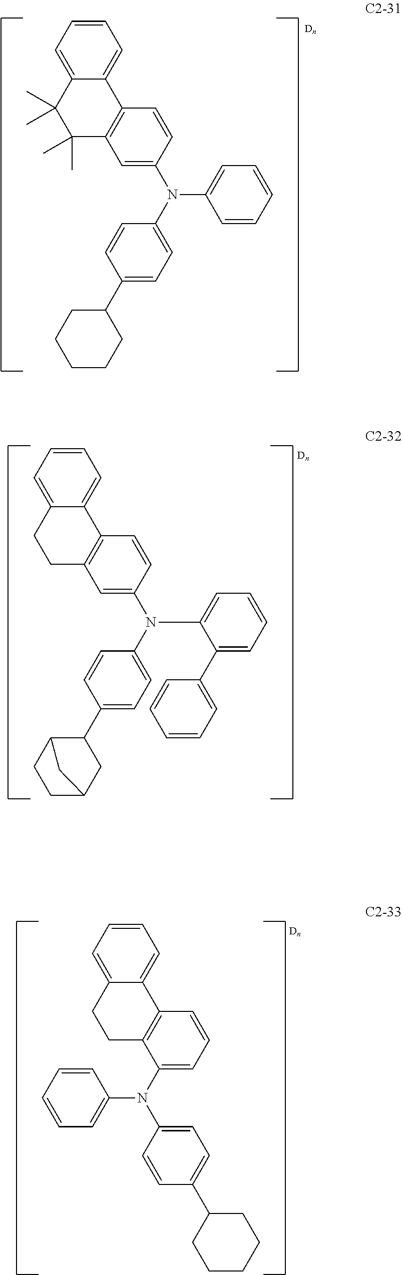

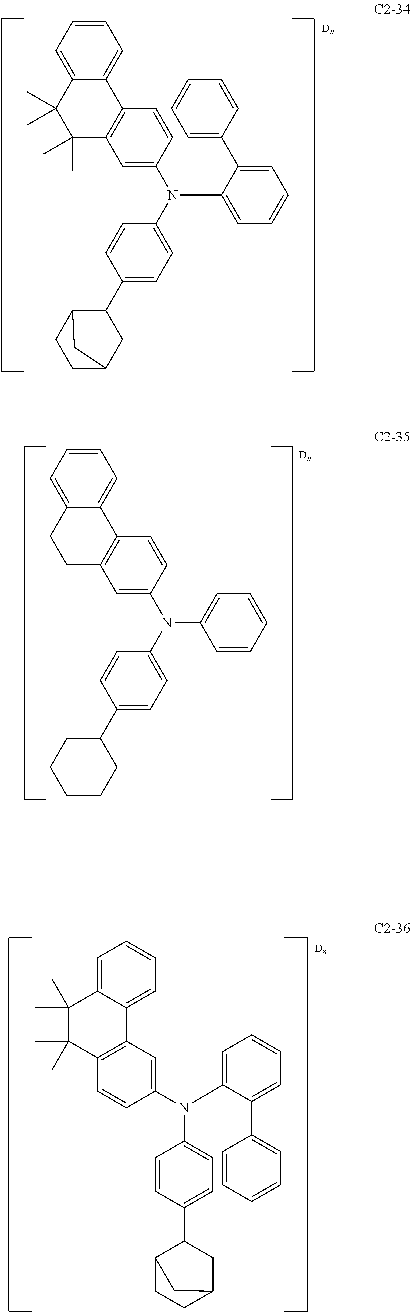

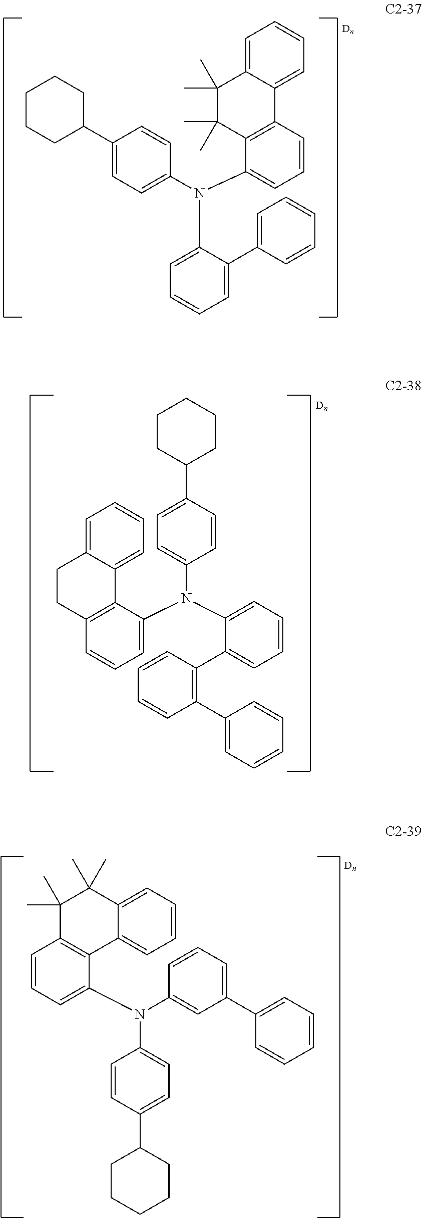

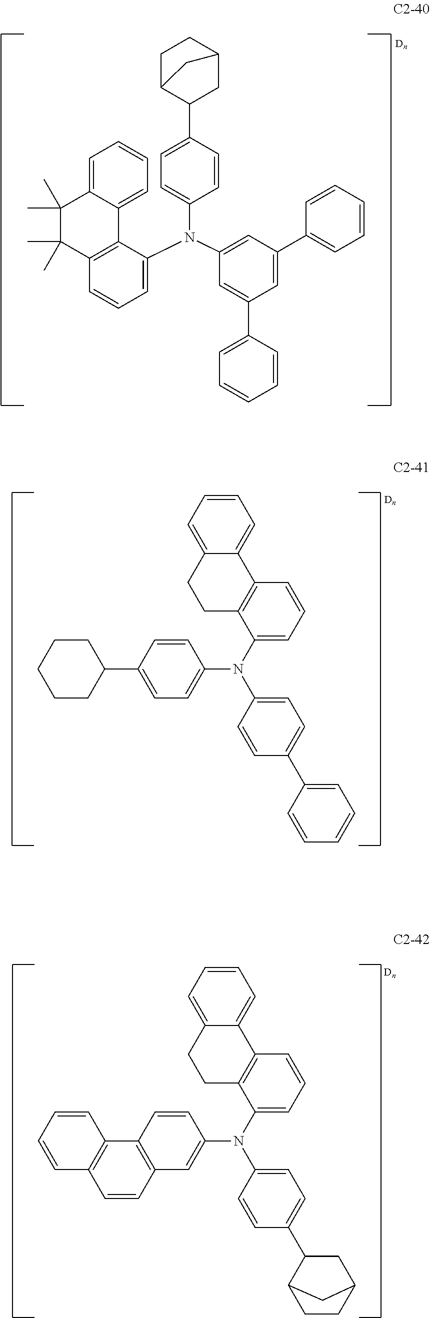

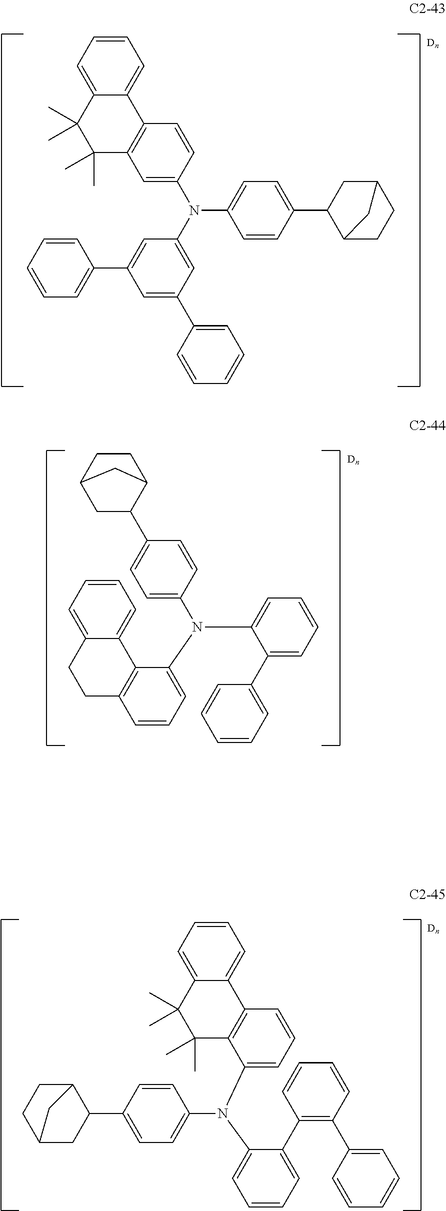

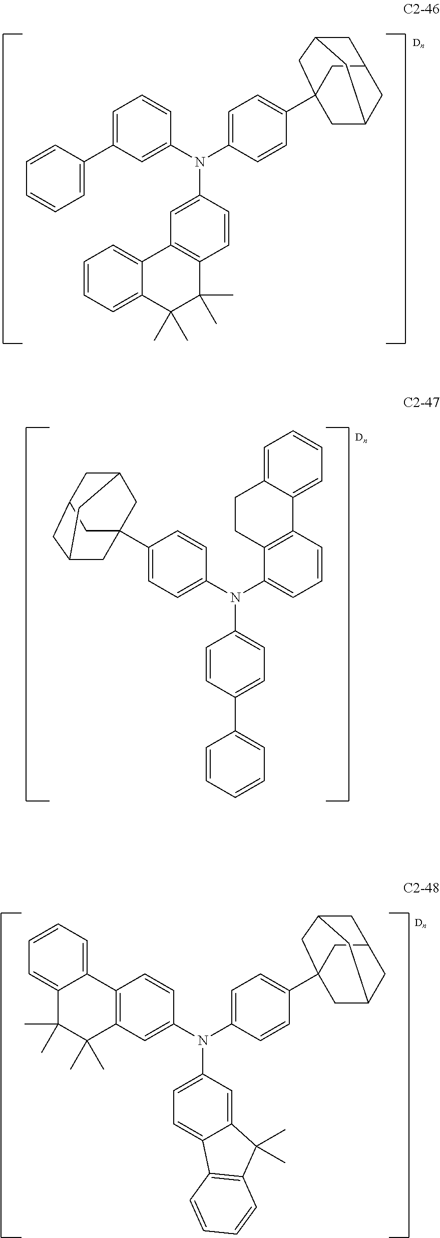

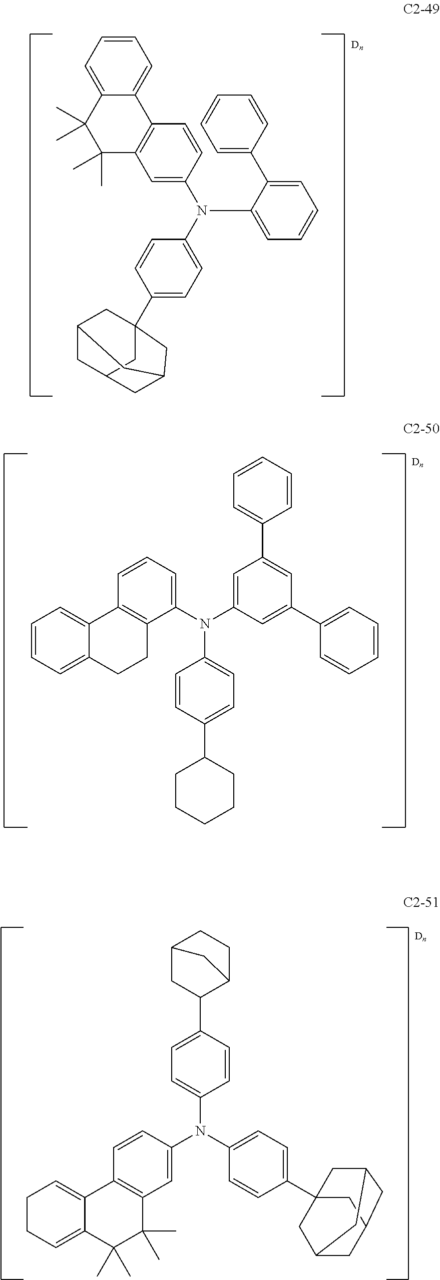

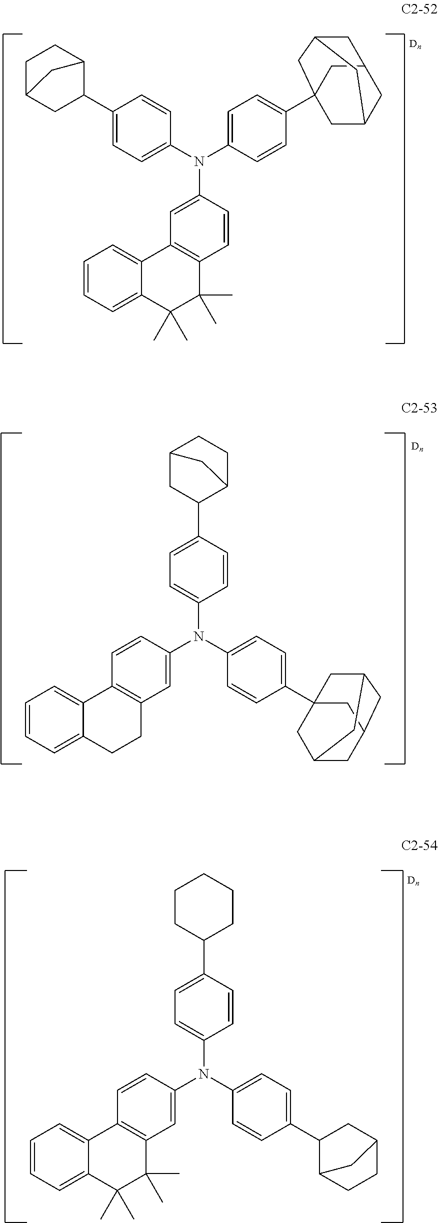

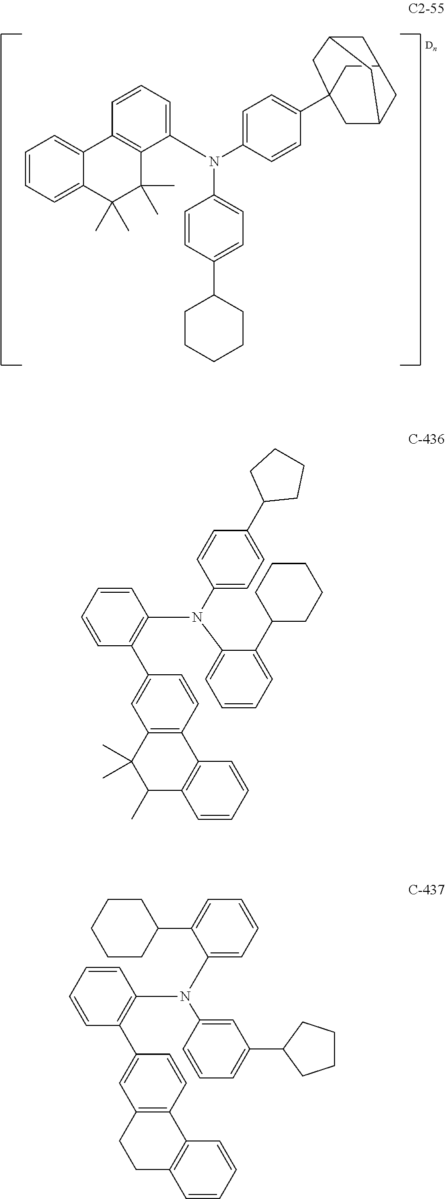

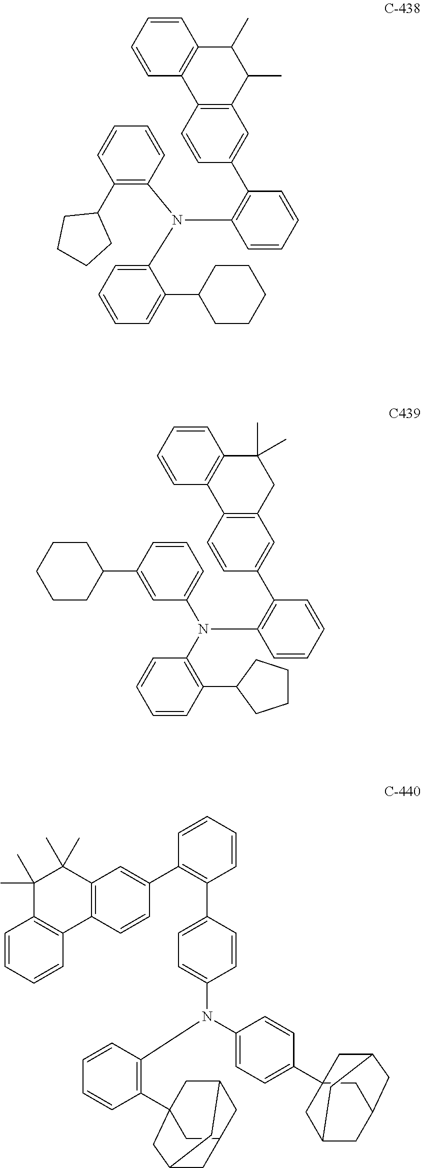 [SMILES: CC1c2ccccc2-c2ccc(-c3ccccc3N(c3ccccc3C3CCCCC3)c3ccccc3C3CCCC3)cc2C1C ; CC1(C)Cc2cc(-c3ccccc3N(c3cccc(C4CCCCC4)c3)c3ccccc3C3CCCC3)ccc2-c2ccccc21 ; CC1(C)c2ccccc2-c2ccc(-c3ccccc3-c3ccc(N(c4ccc(C56CC7CC(CC(C7)C5)C6)cc4)c4ccccc4C45CC6CC(CC(C6)C4)C5)cc3)cc2C1(C)C]

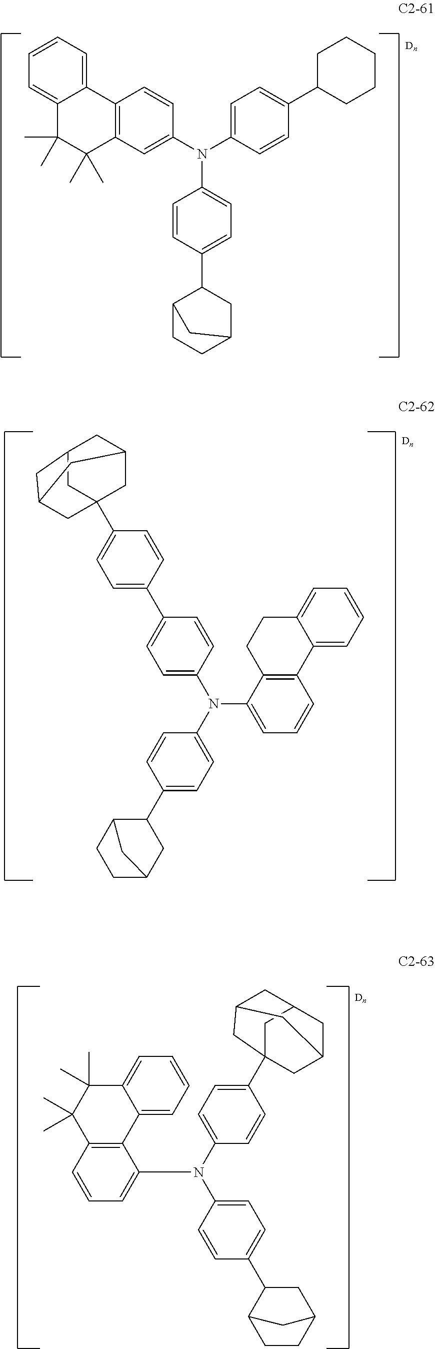

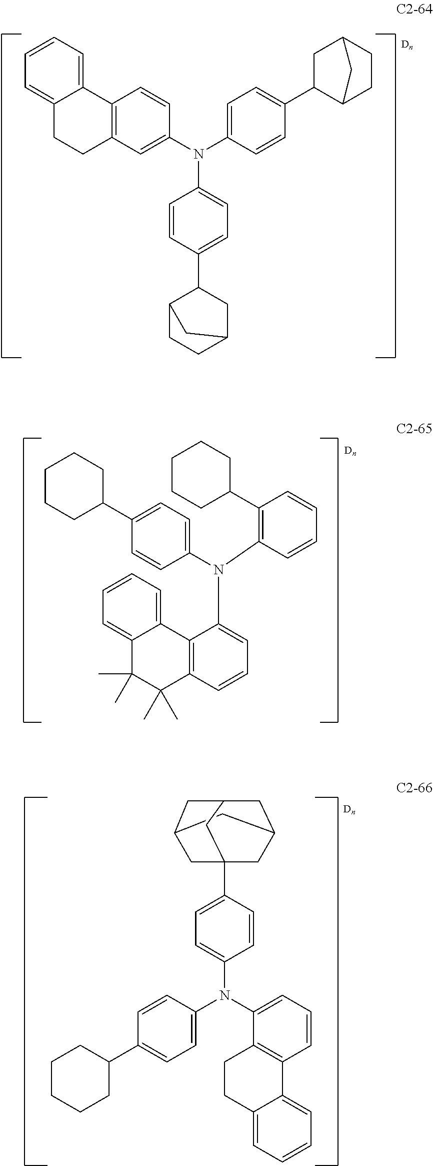

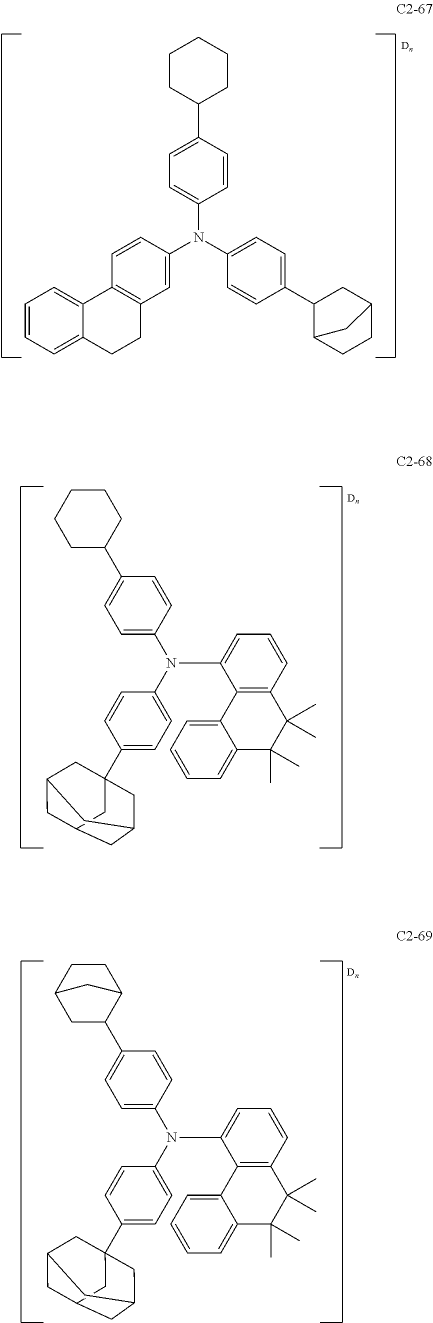

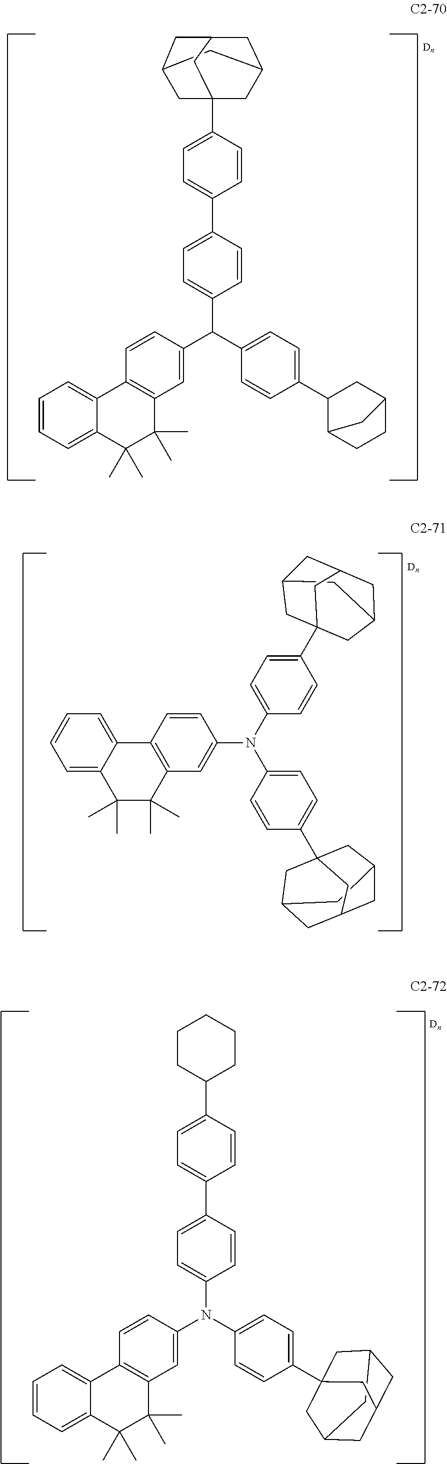

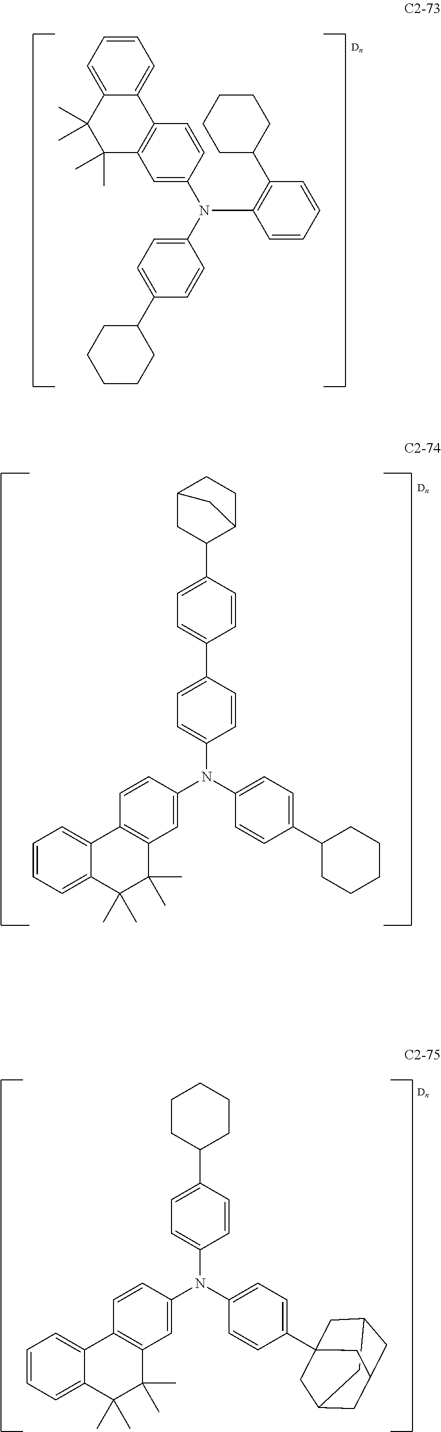

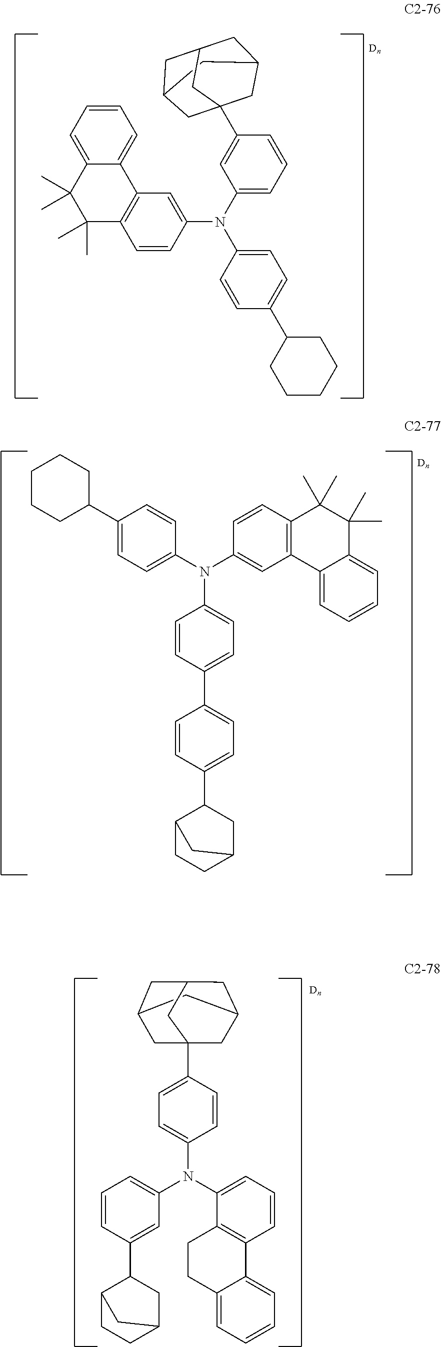

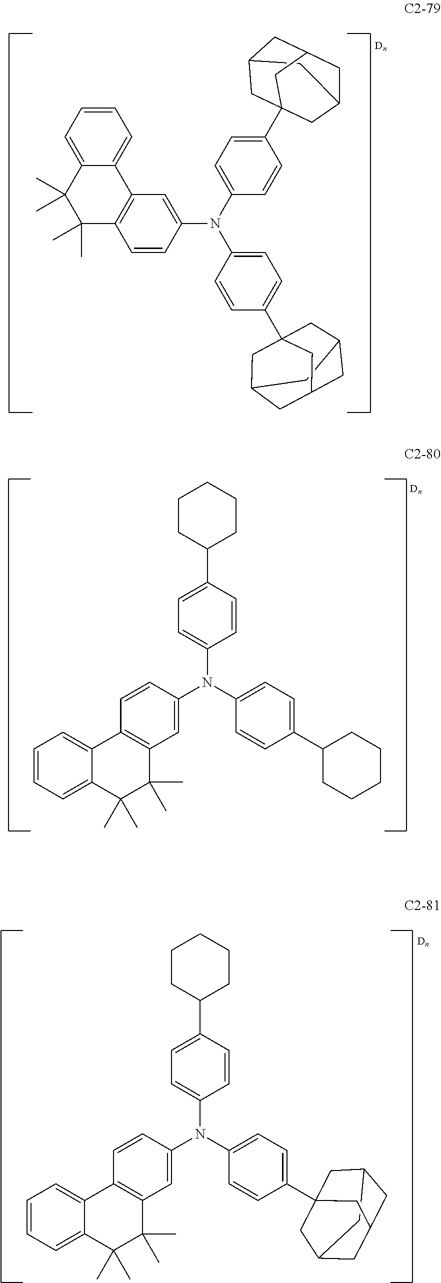

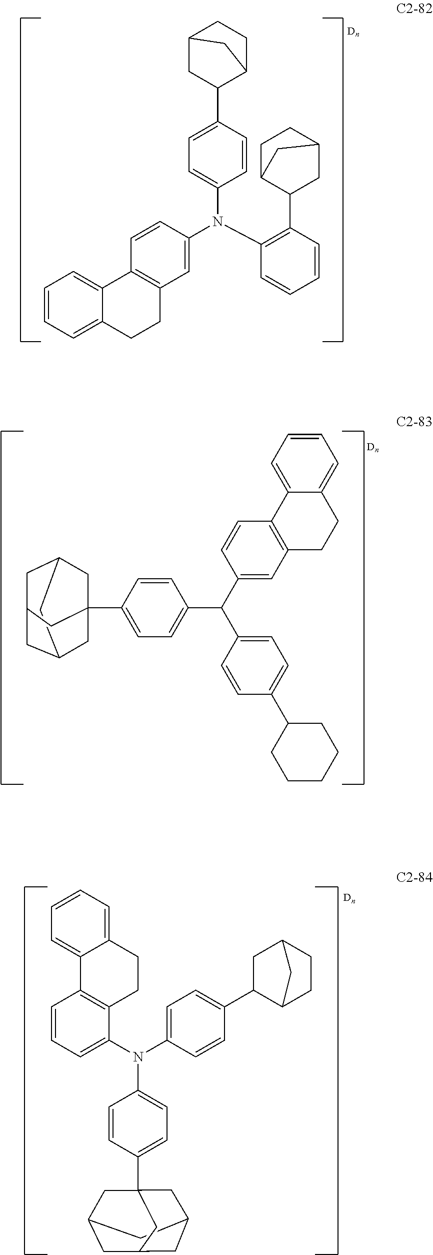

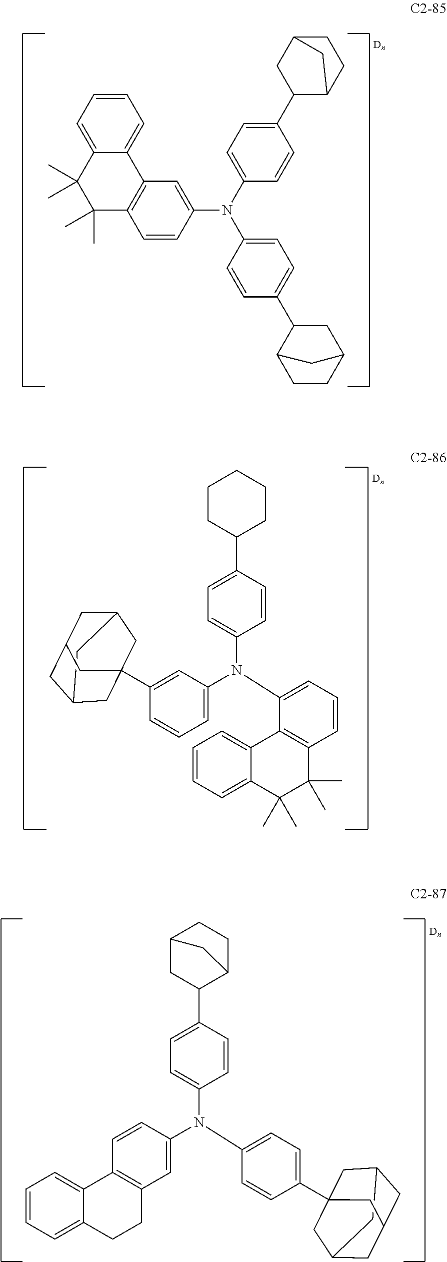

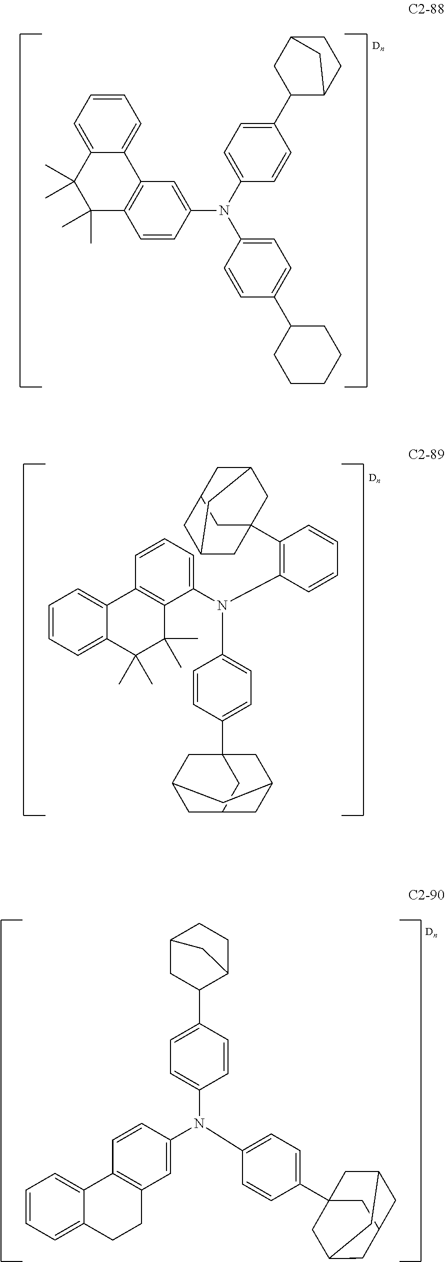

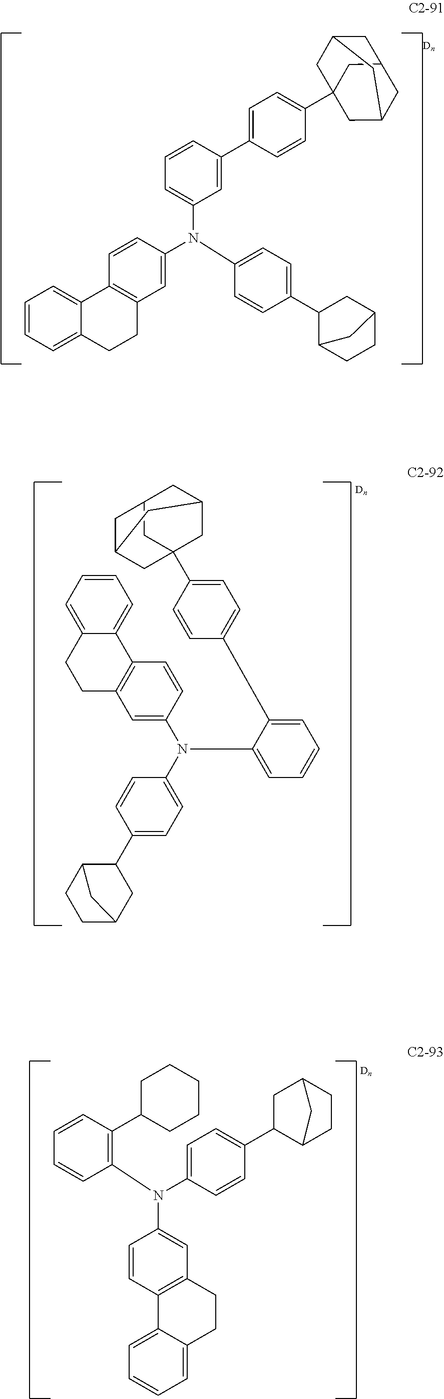

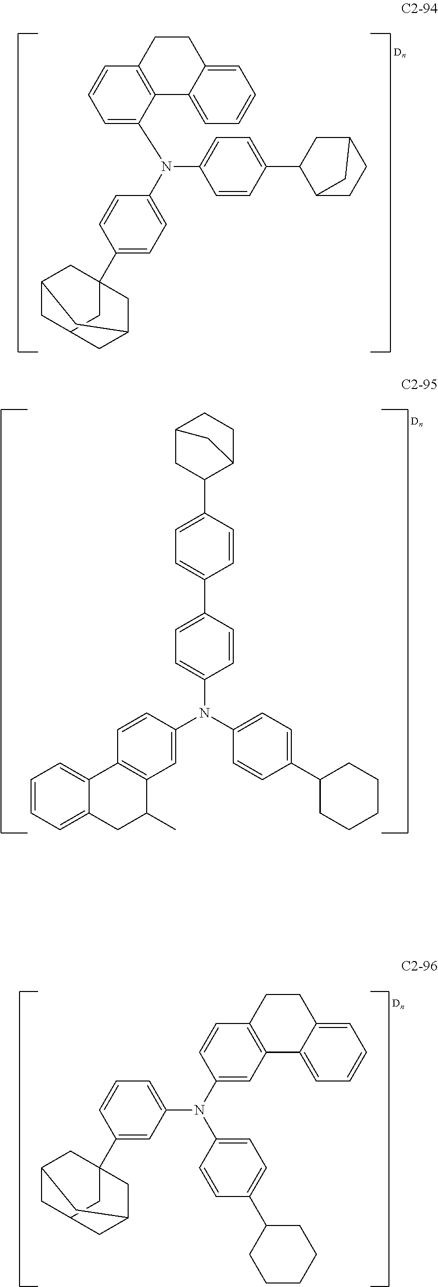

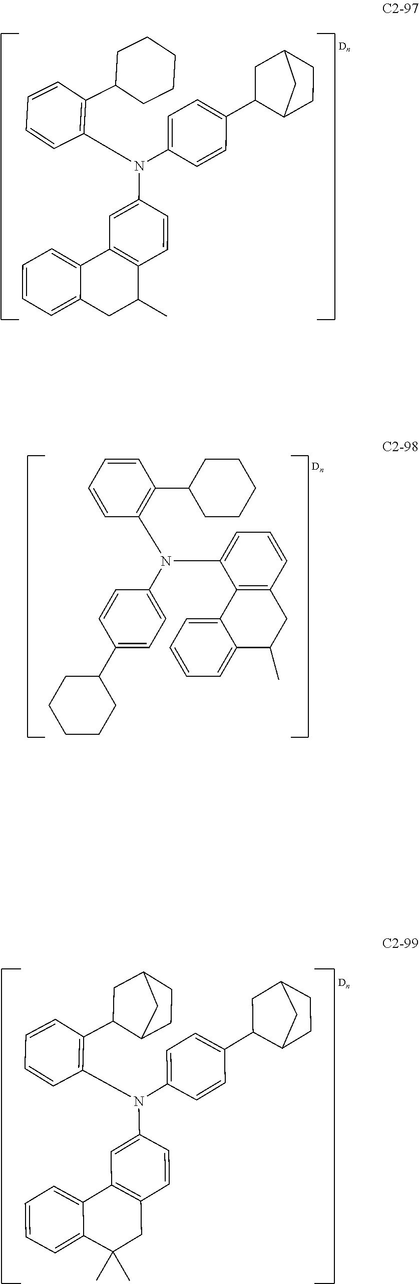

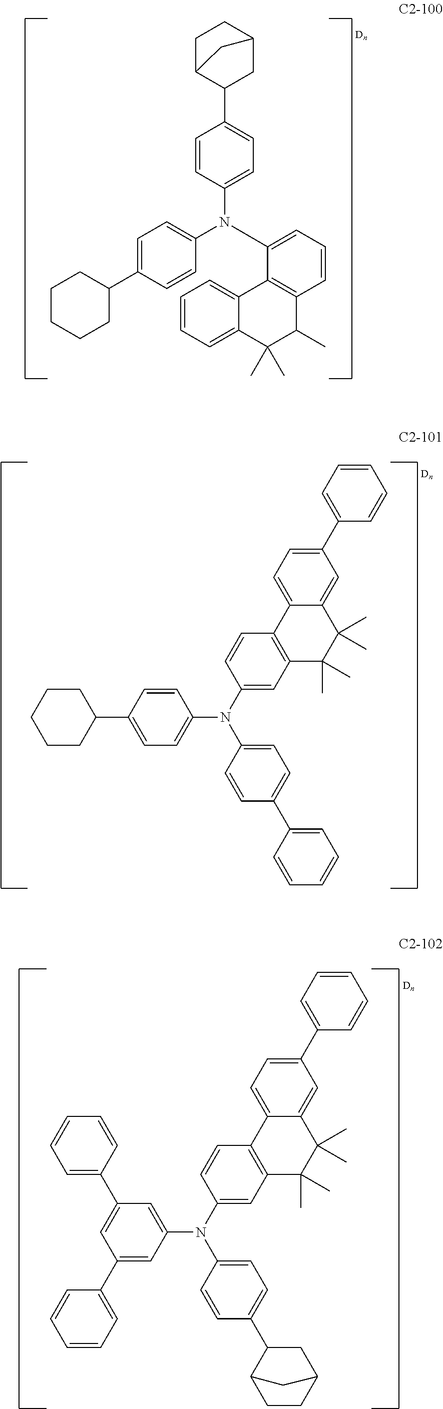

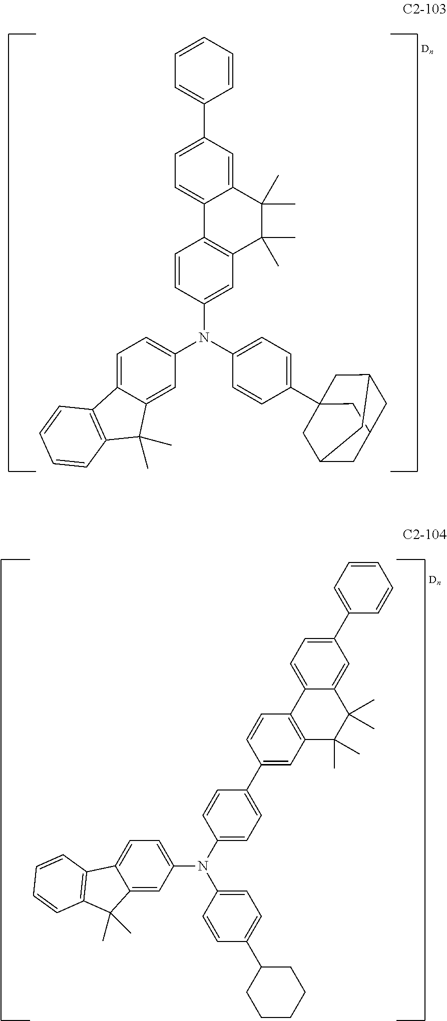

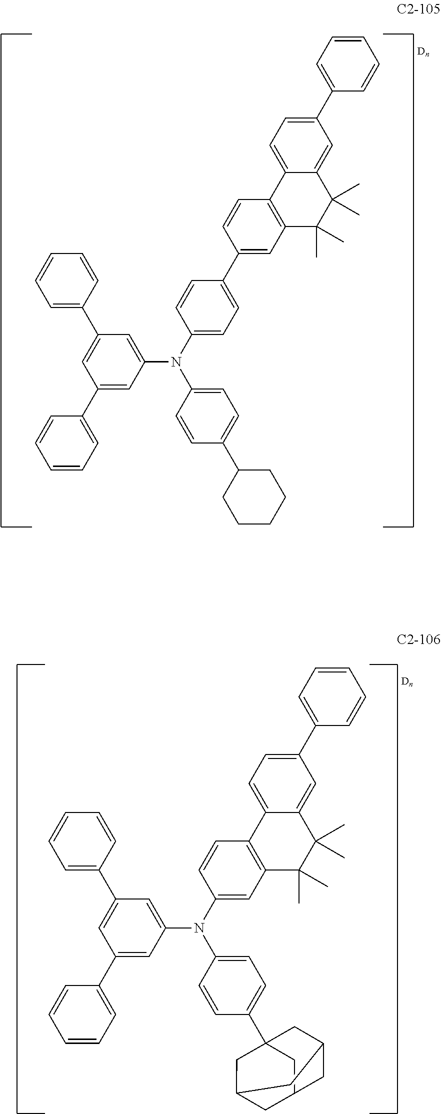

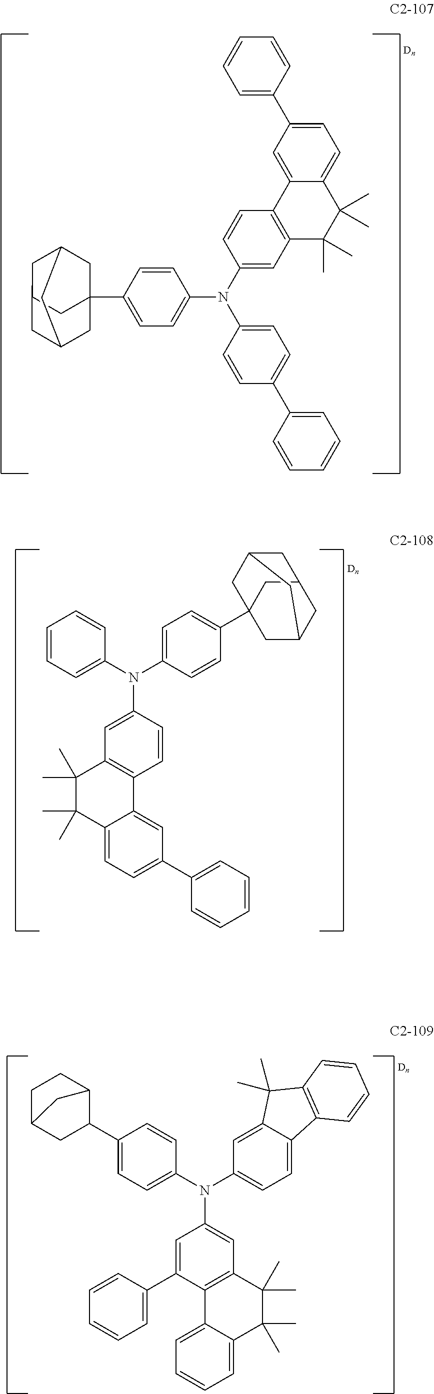

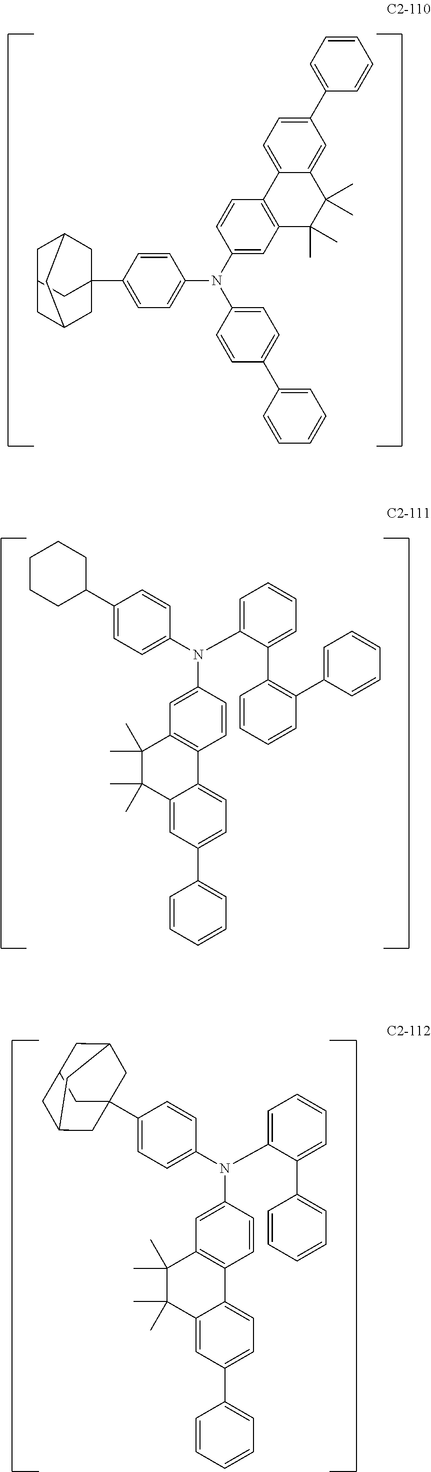

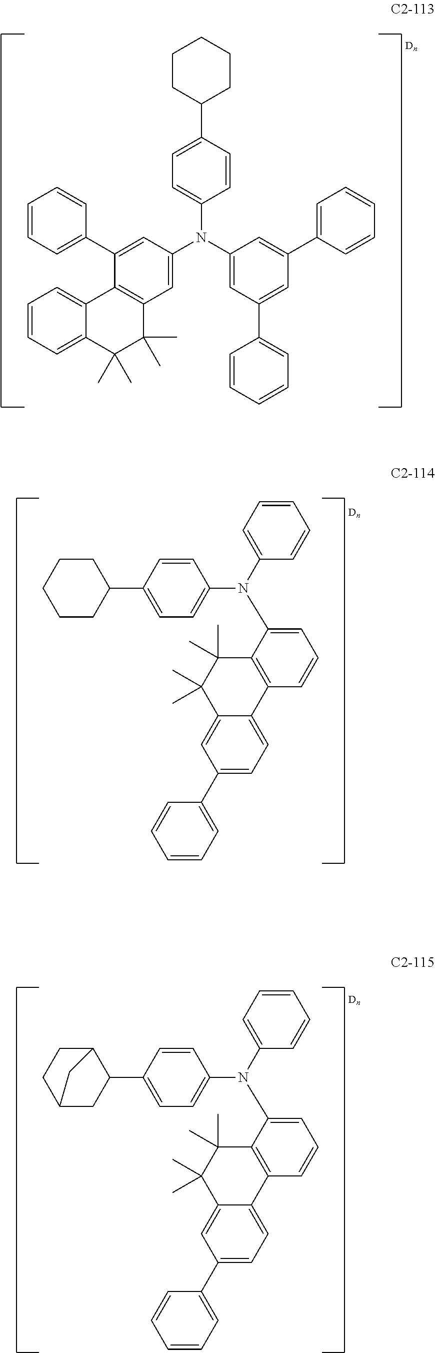

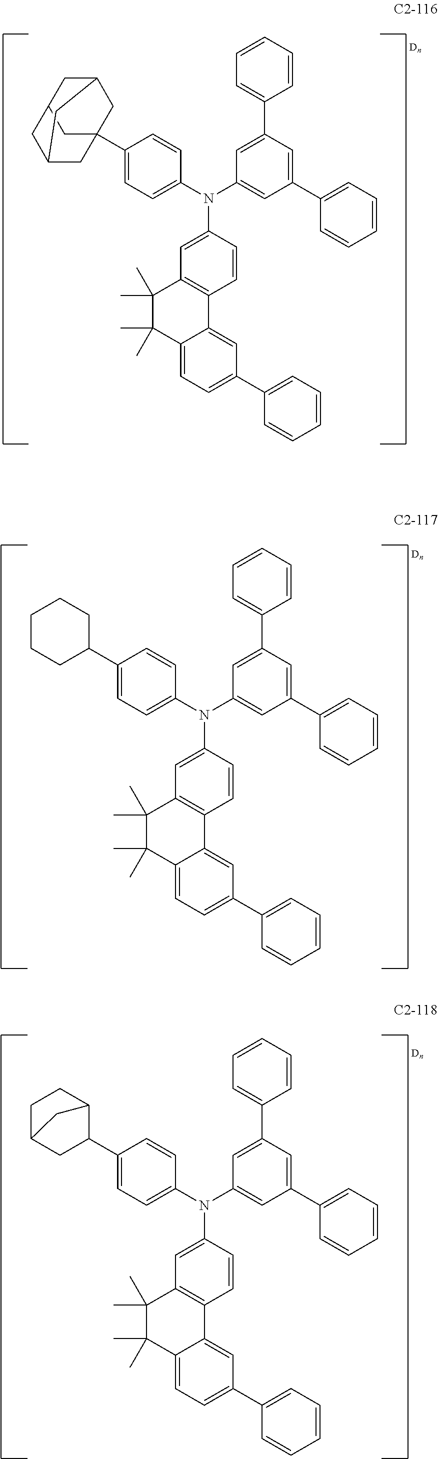

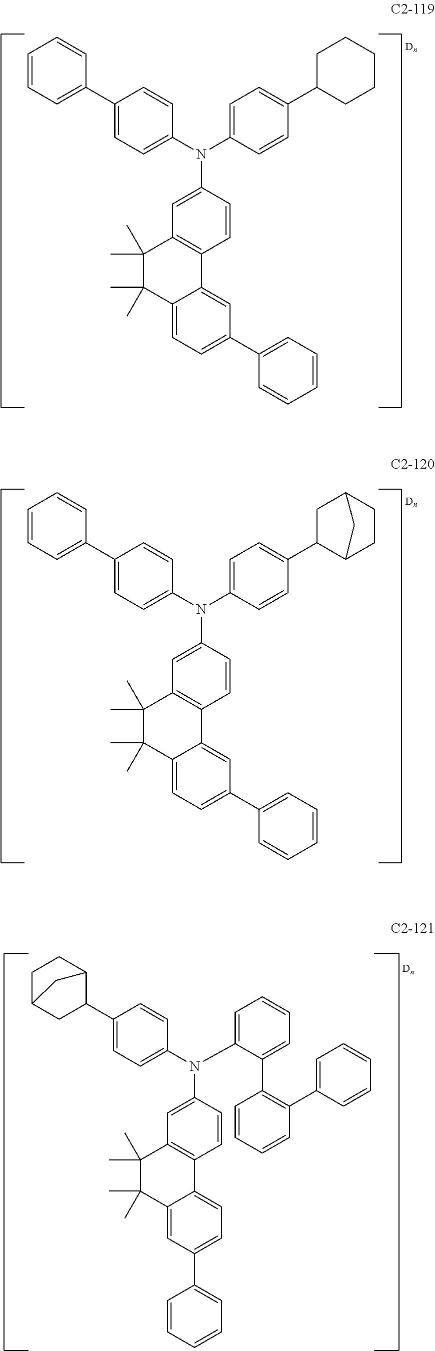

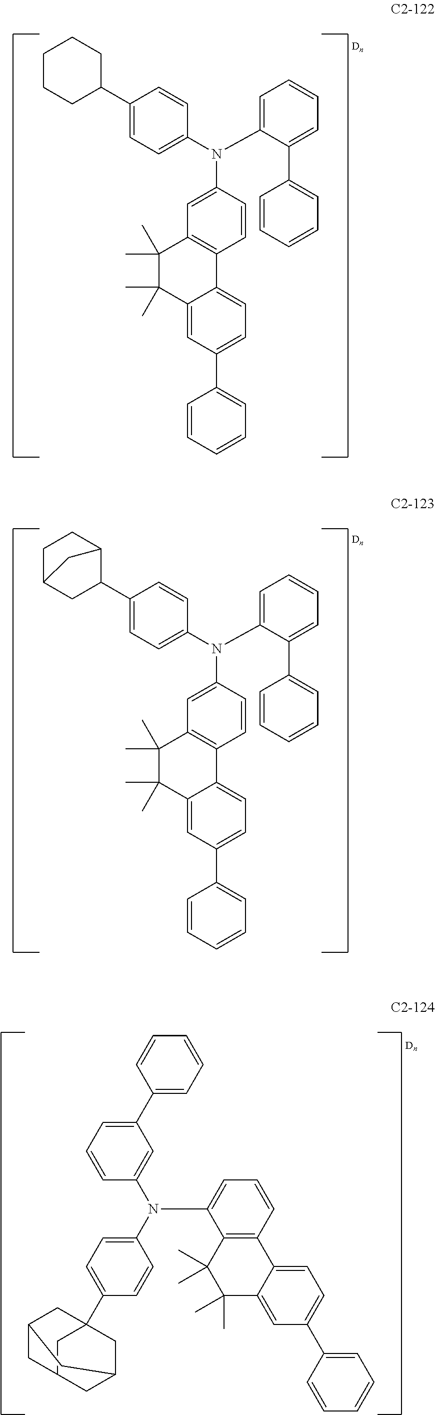

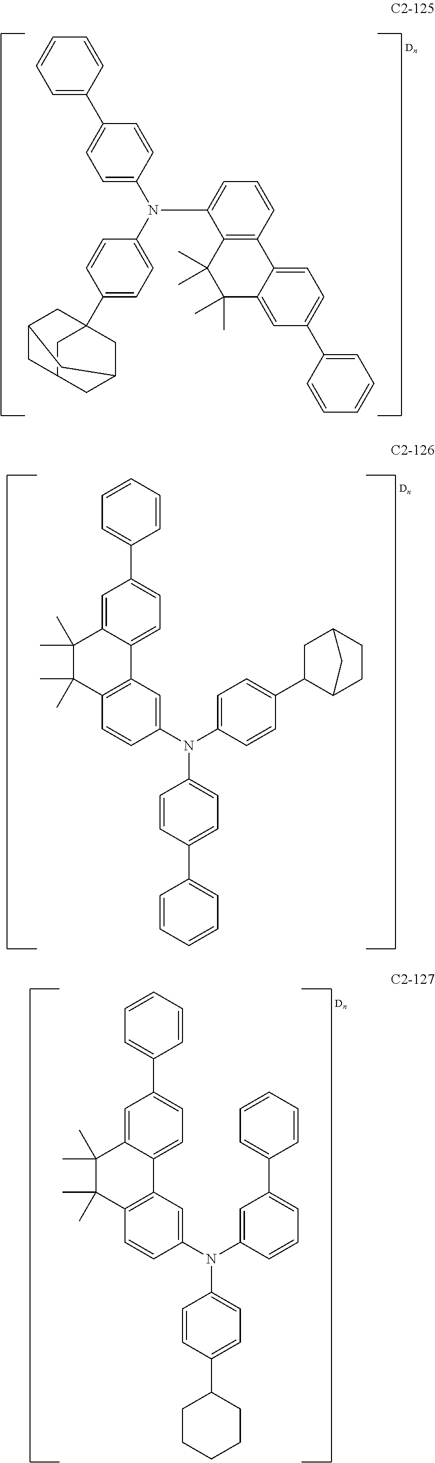

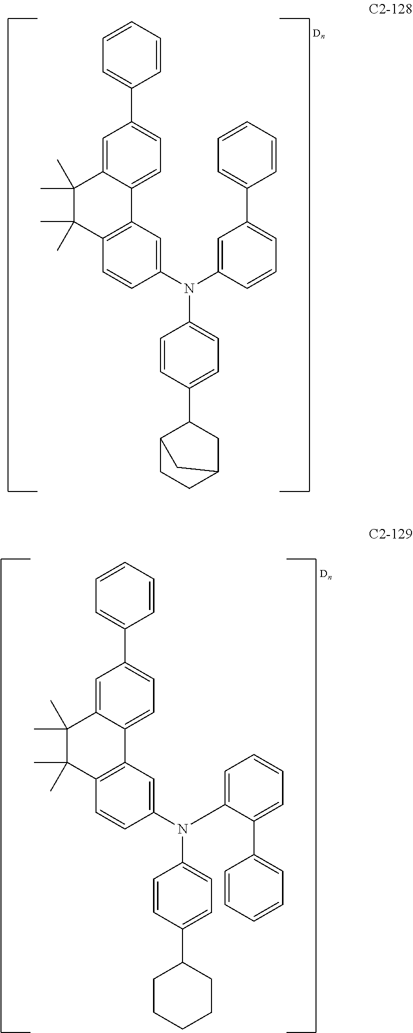

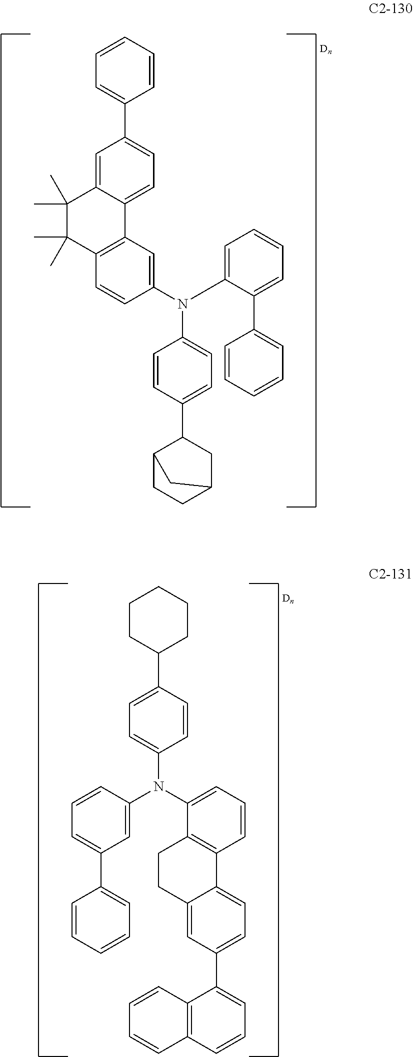

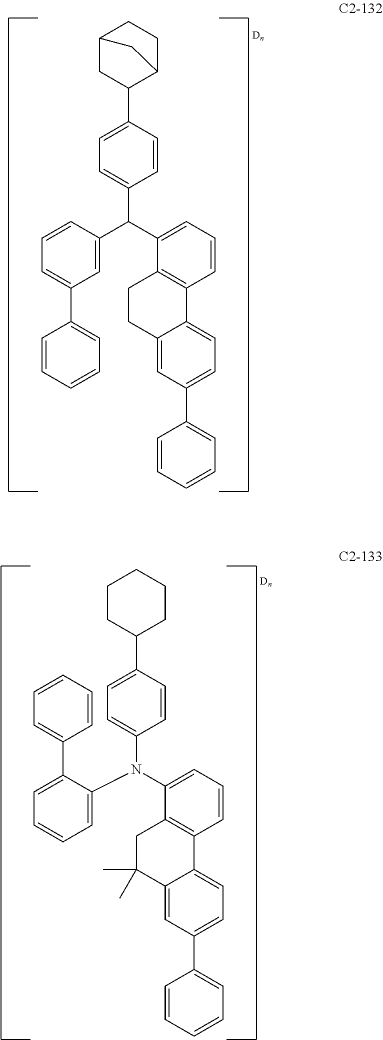

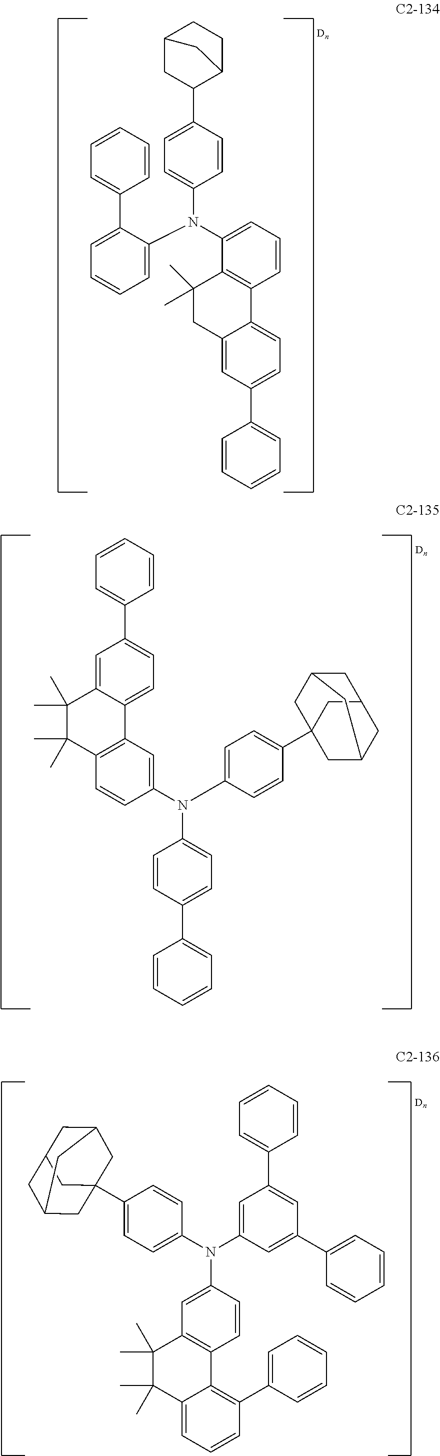

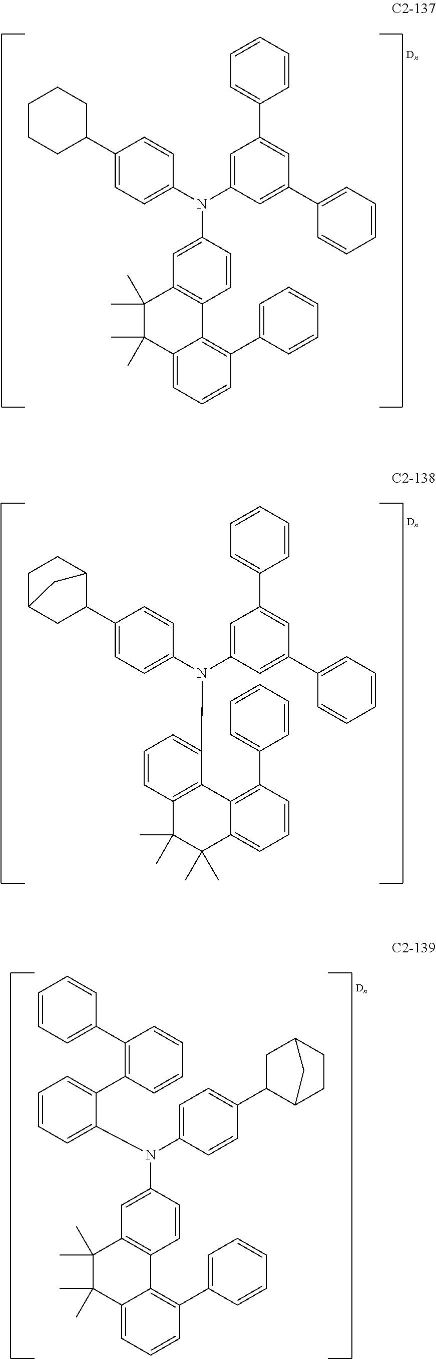

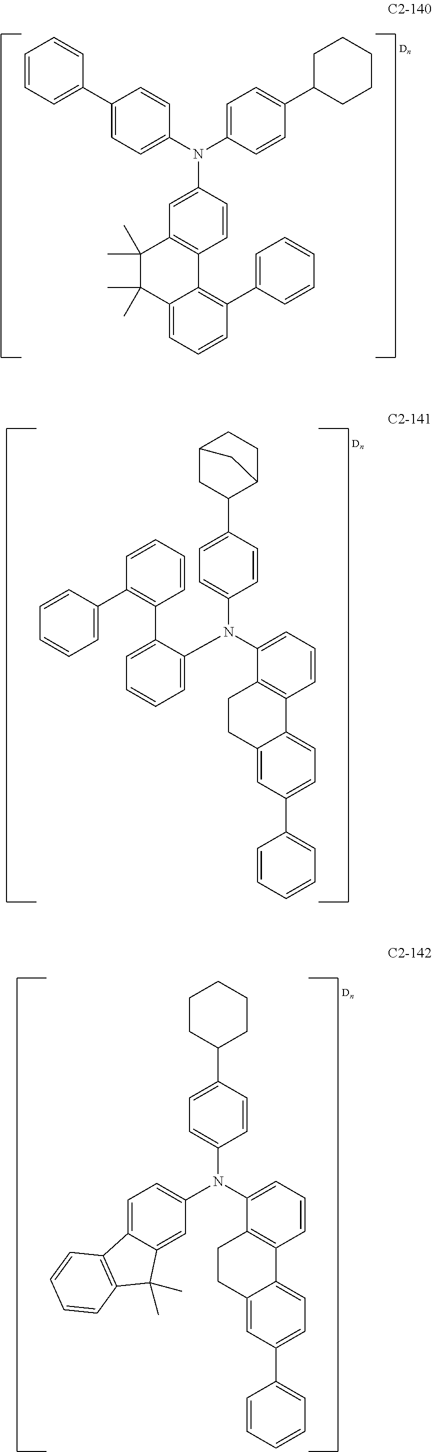

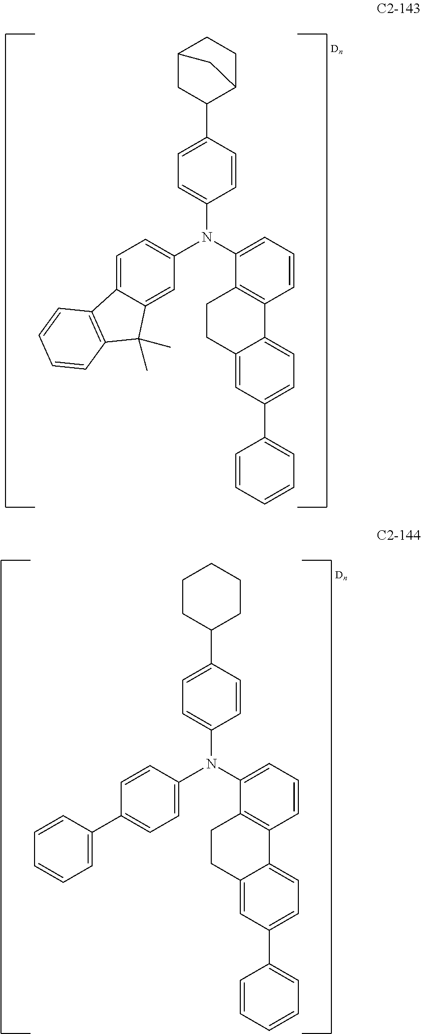

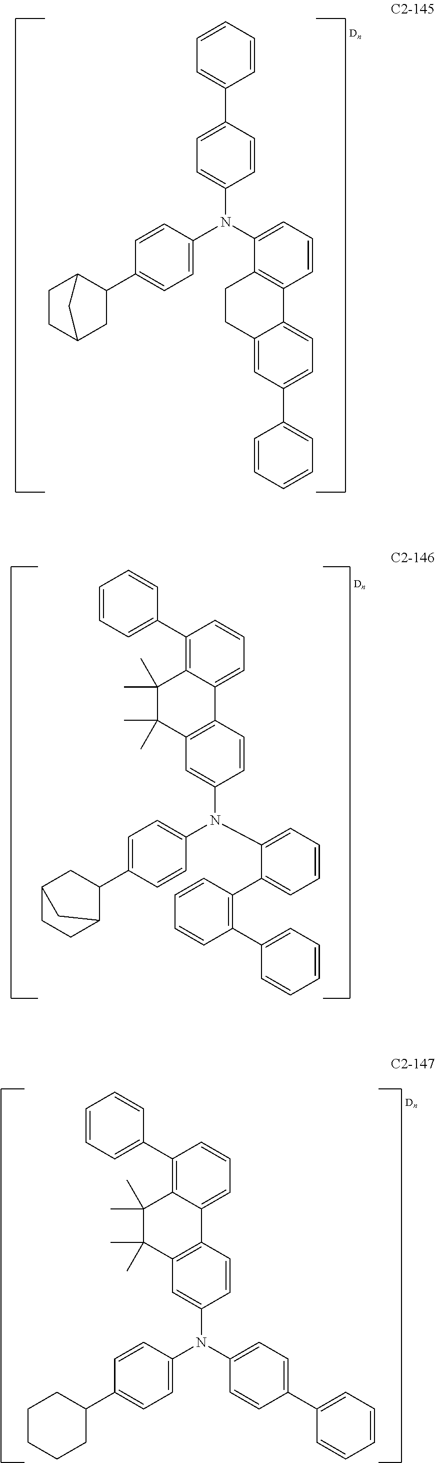

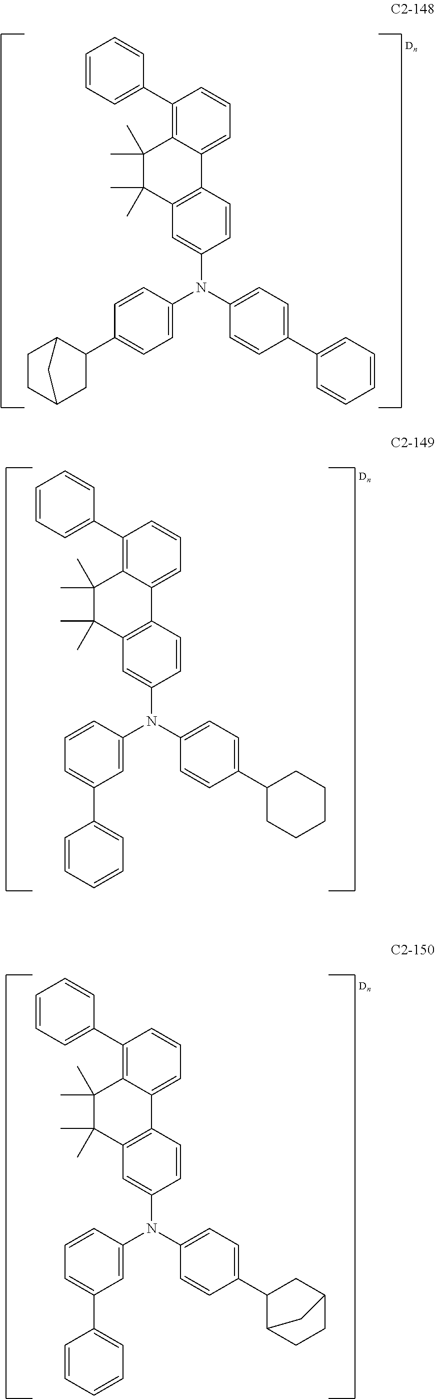

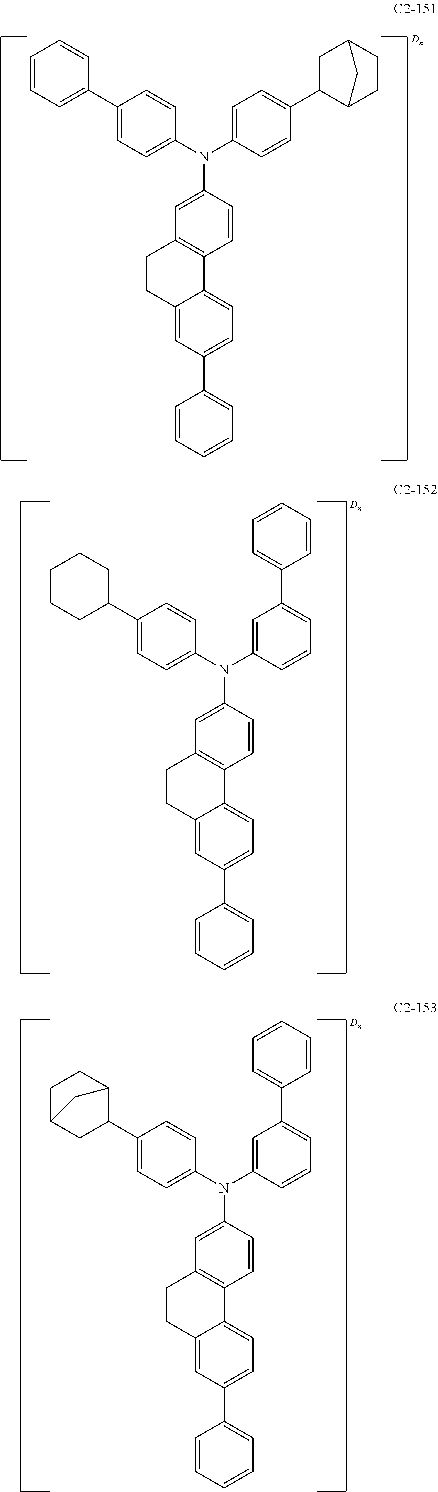

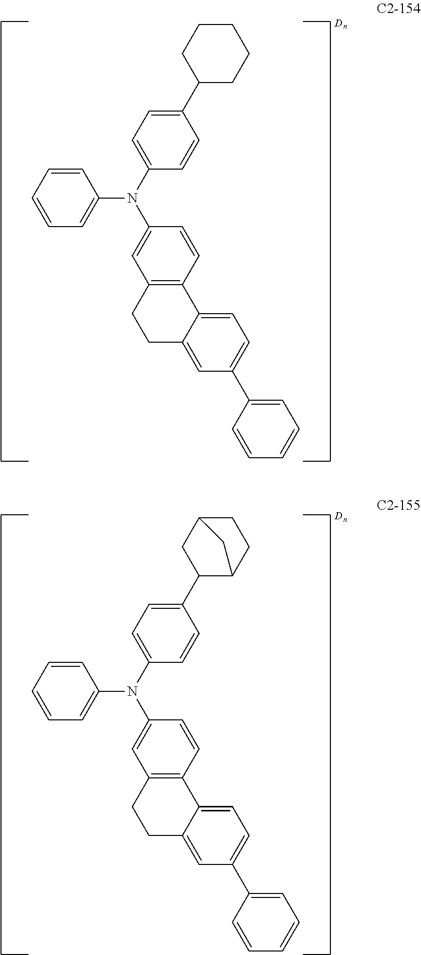

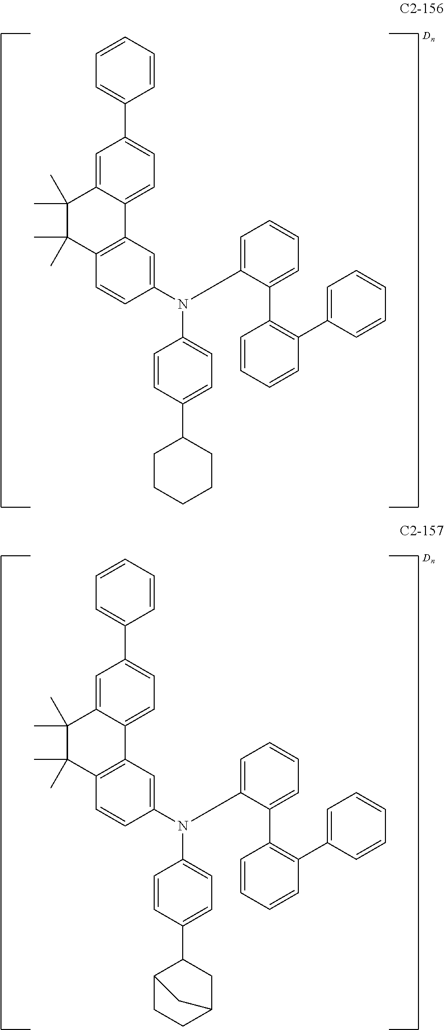

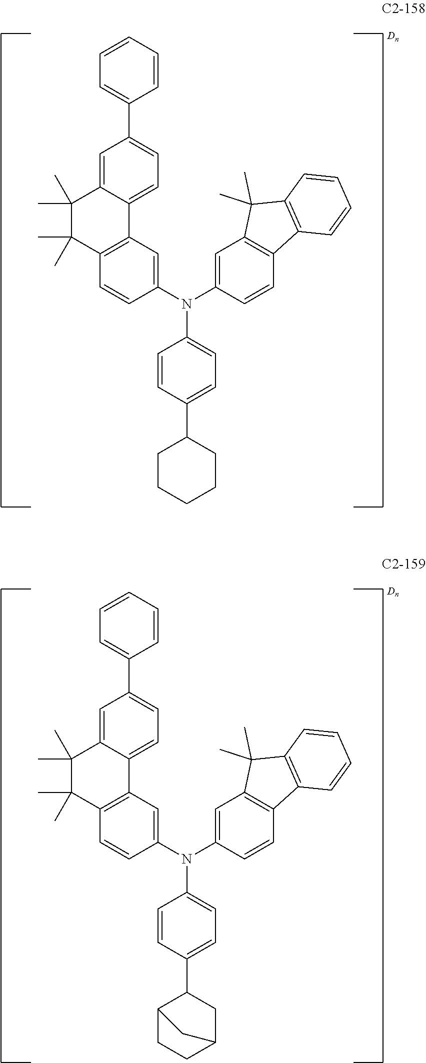

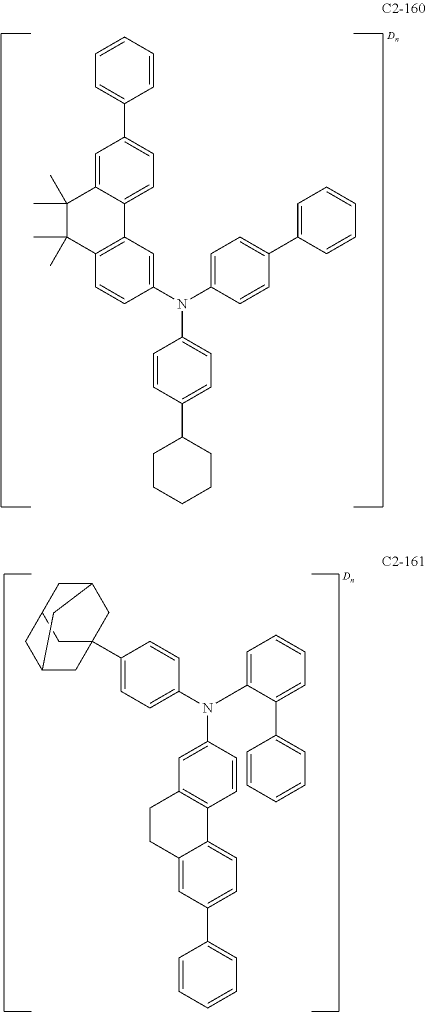

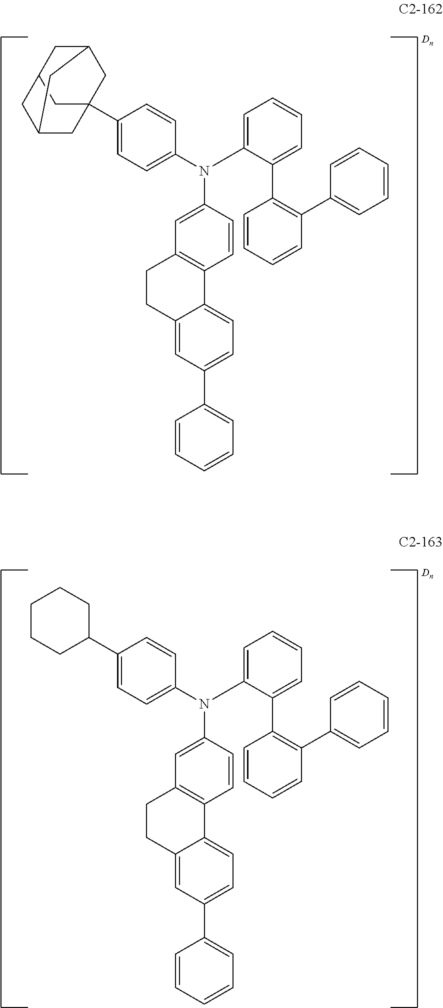

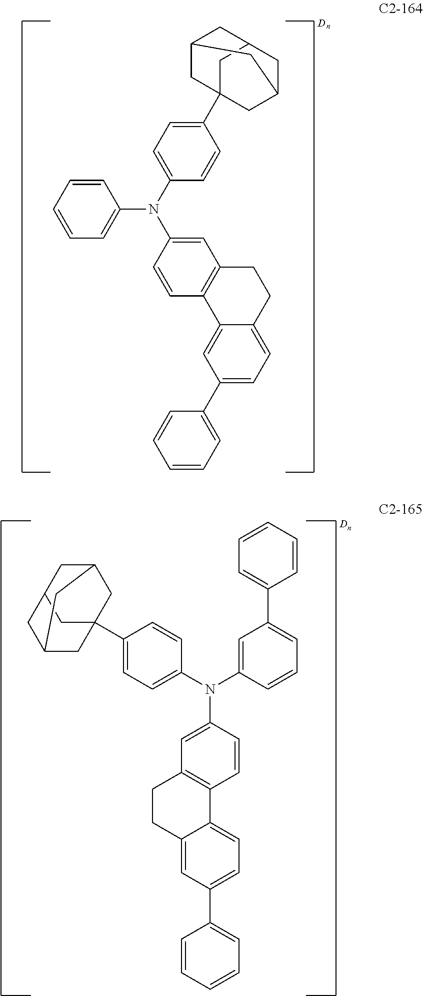

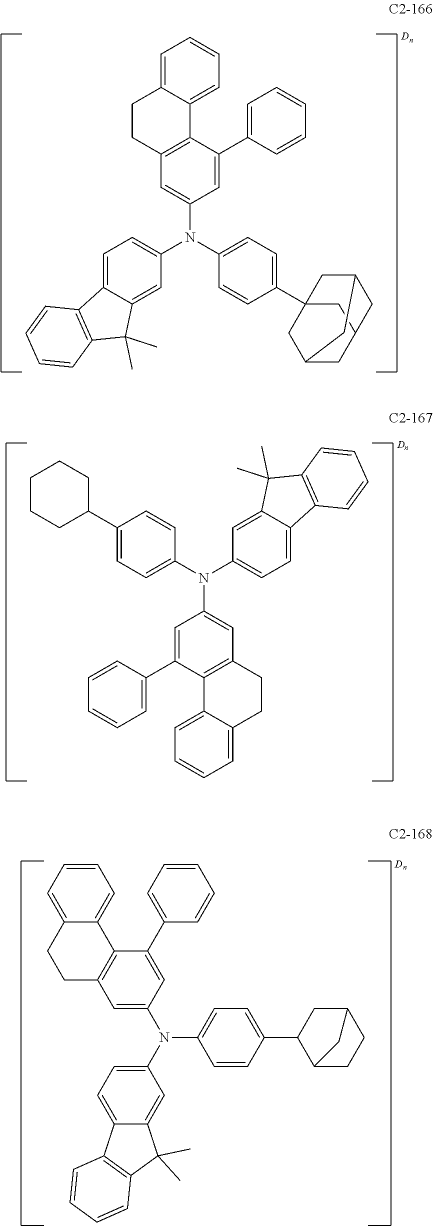

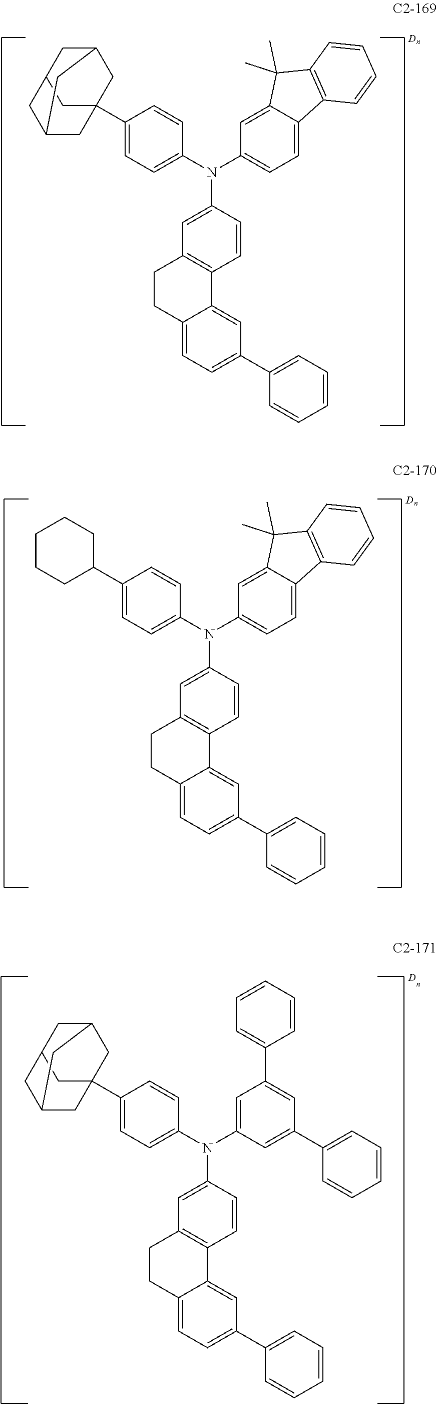

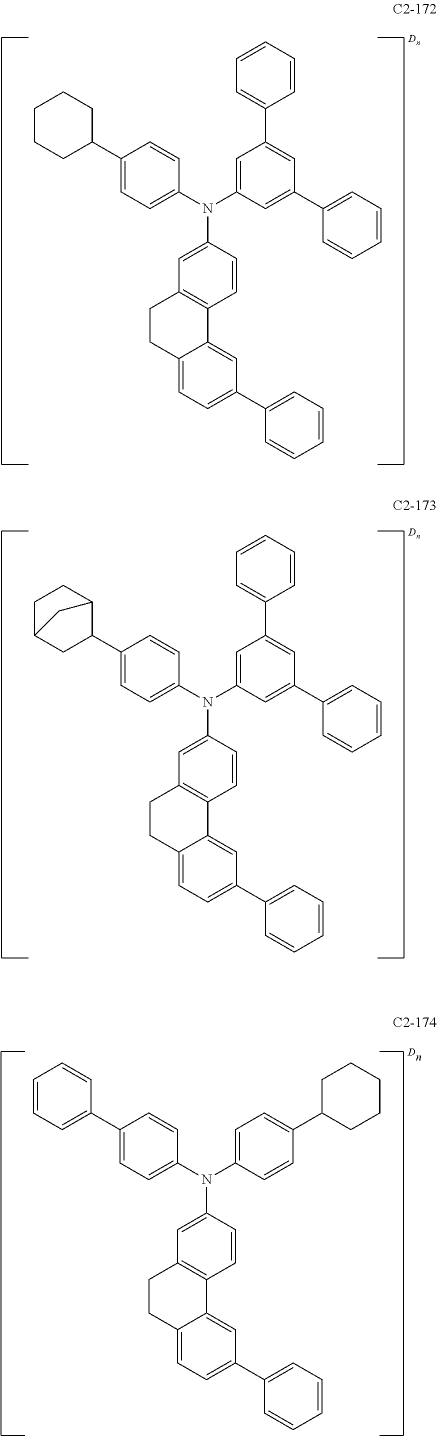

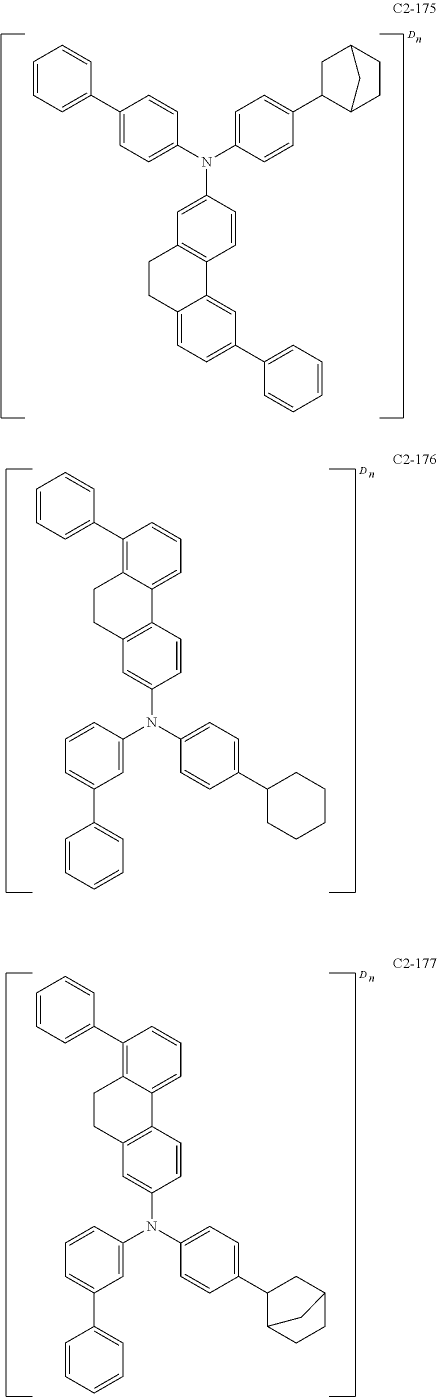

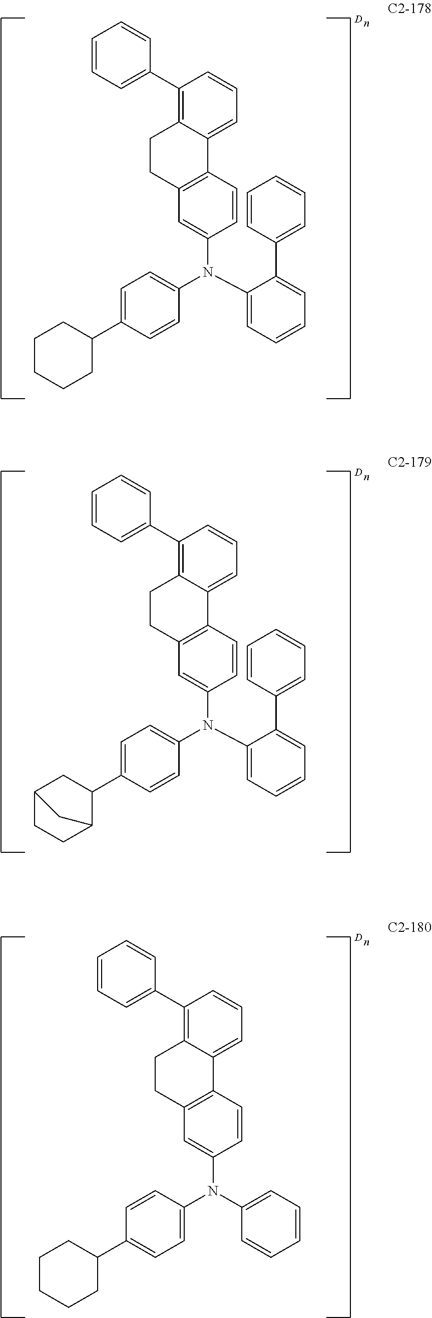

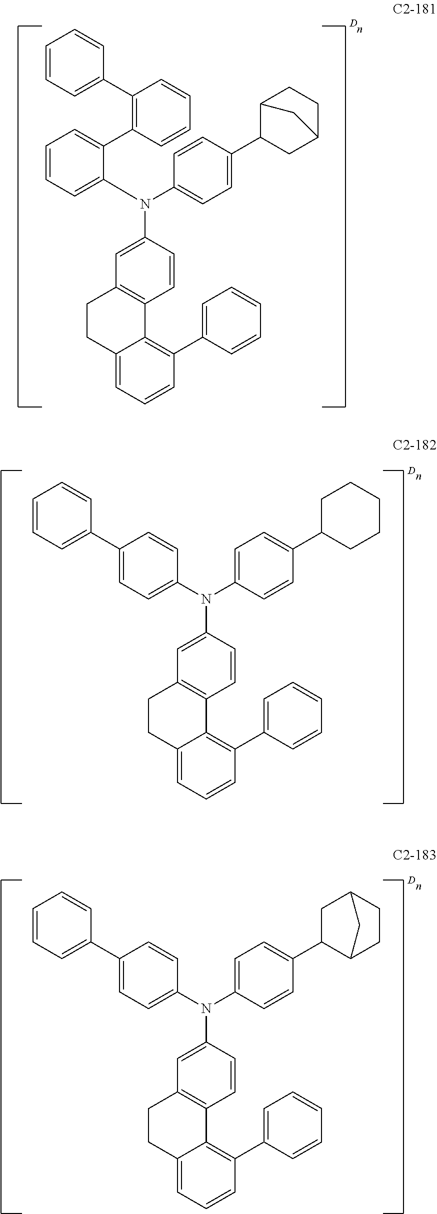

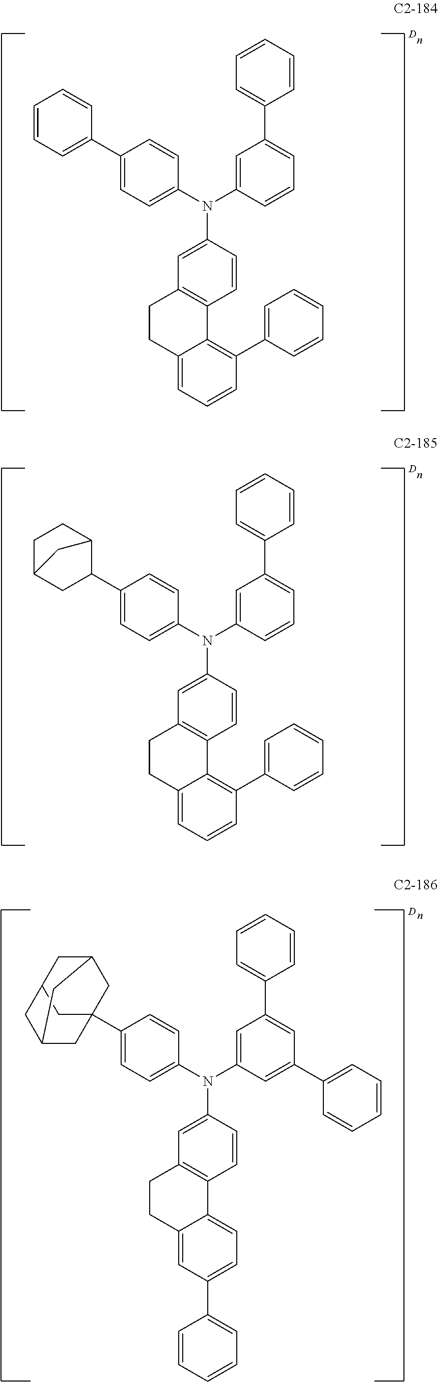

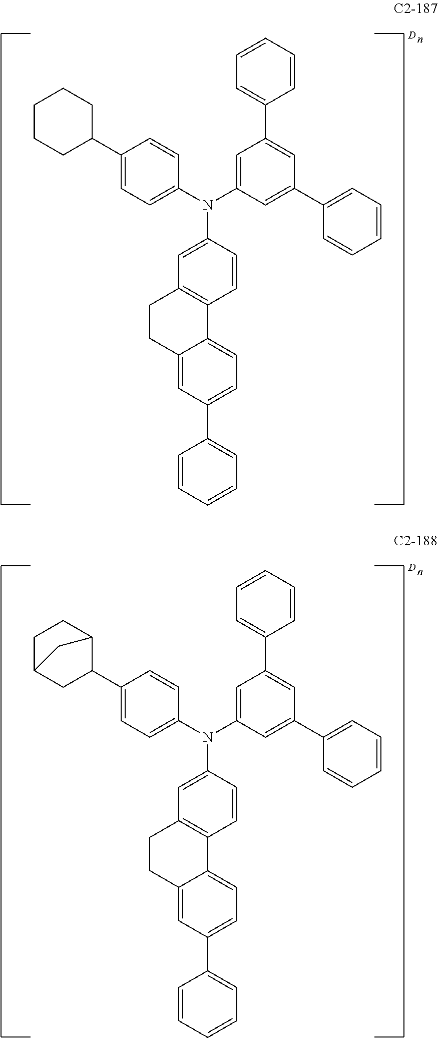

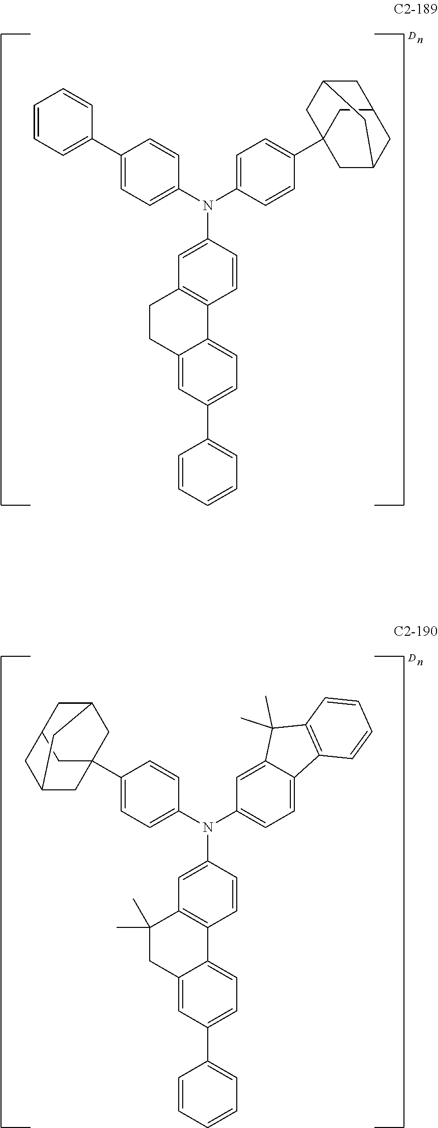

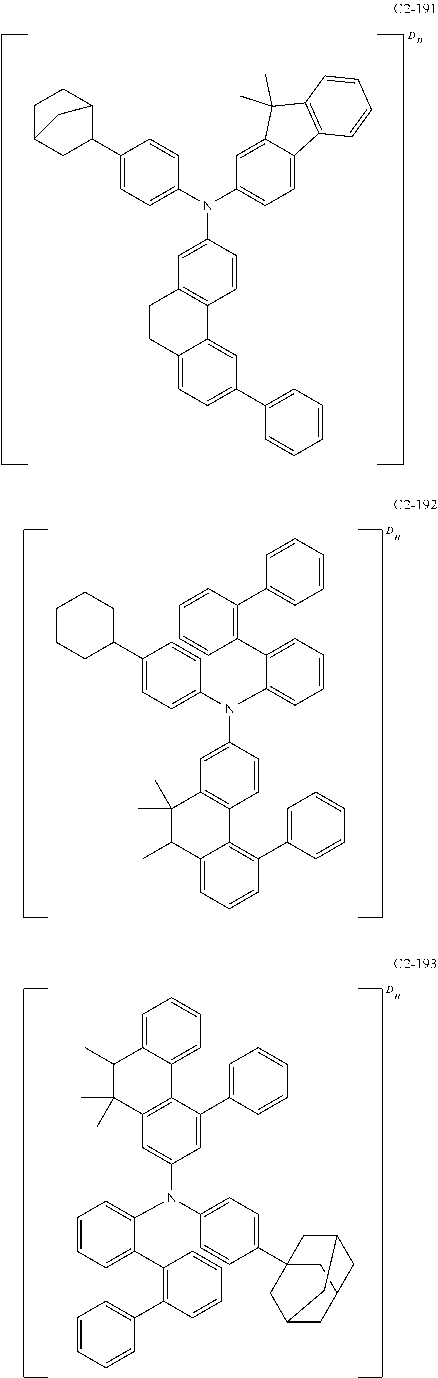

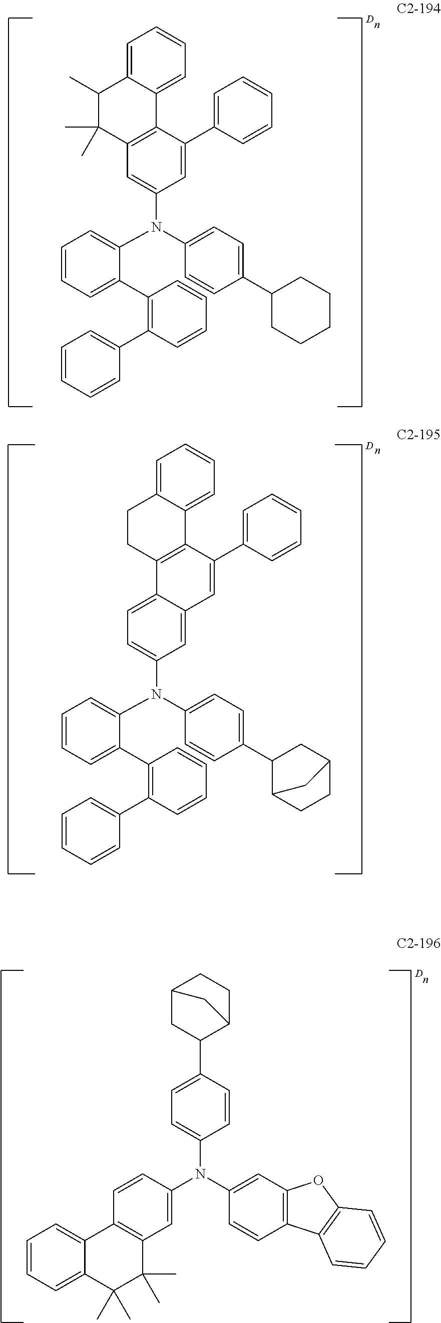

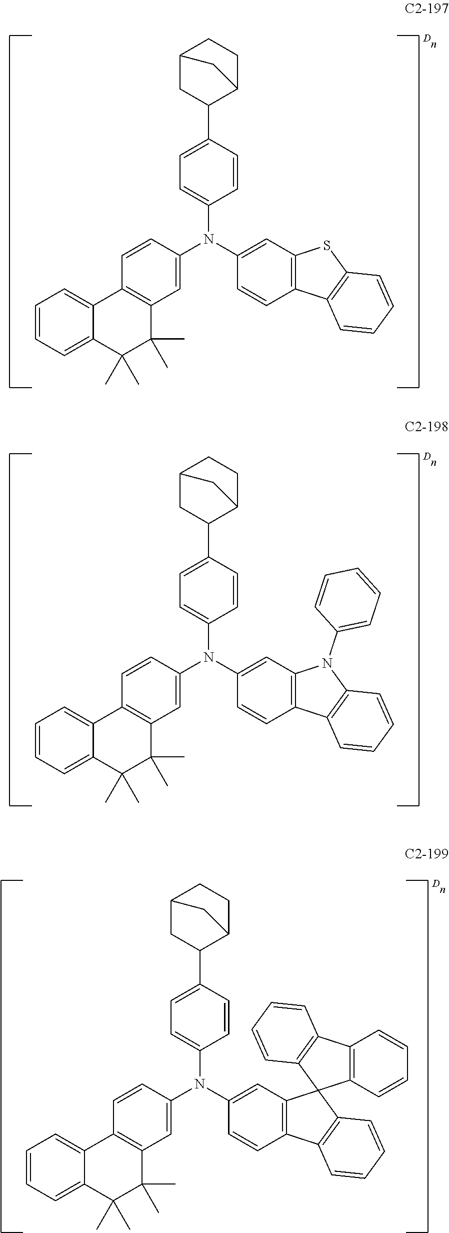

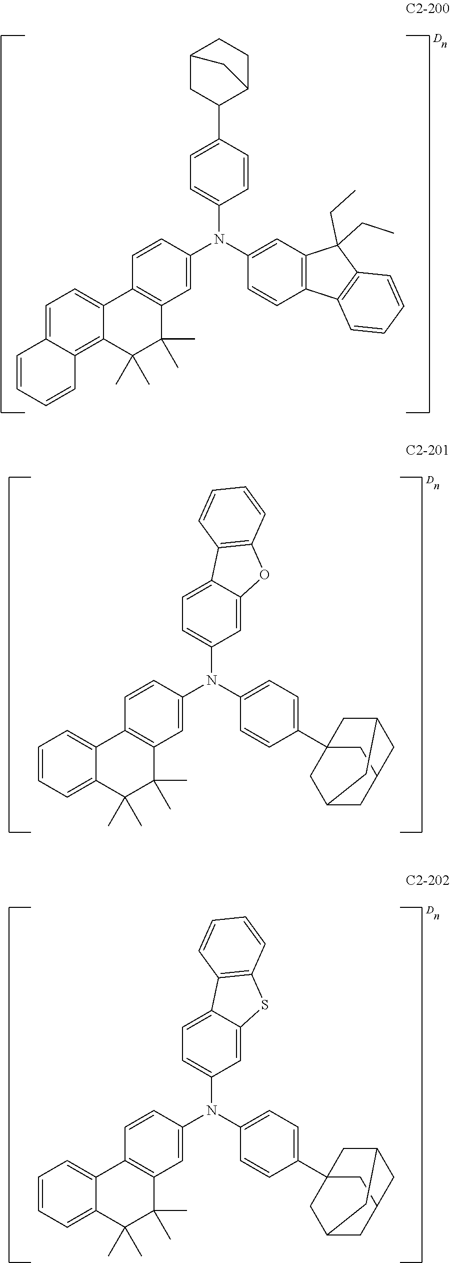

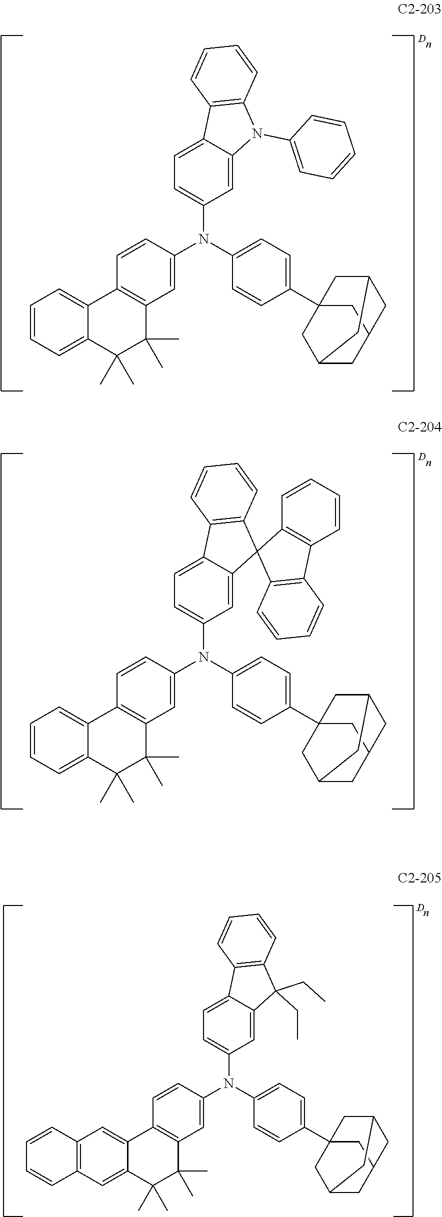

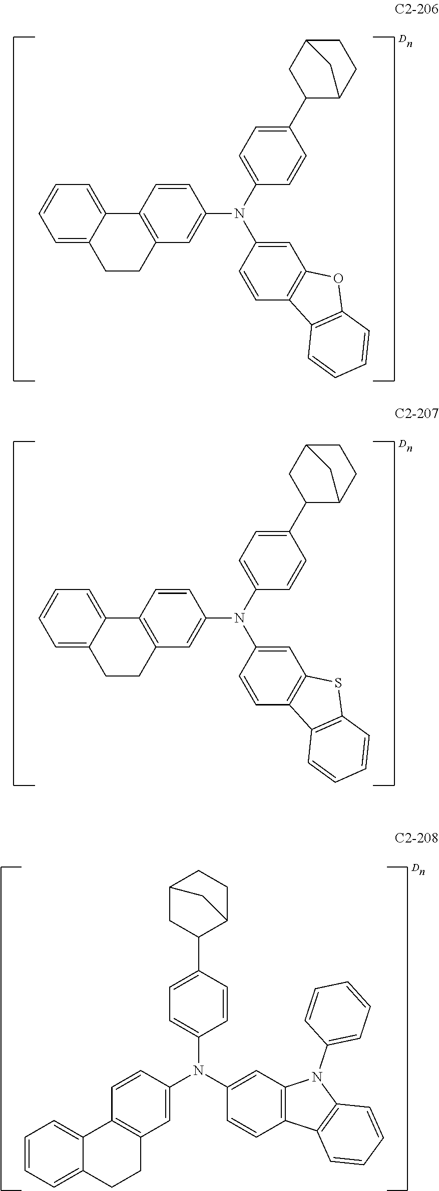

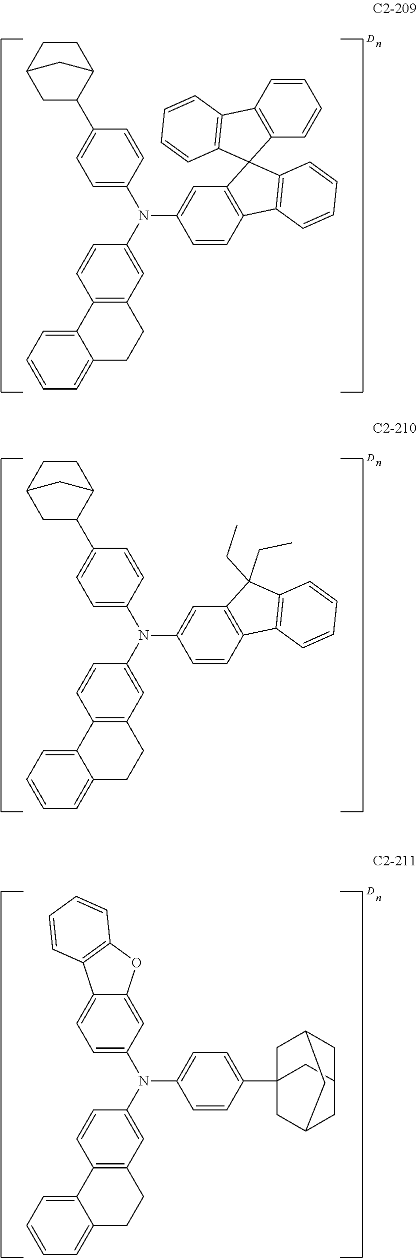

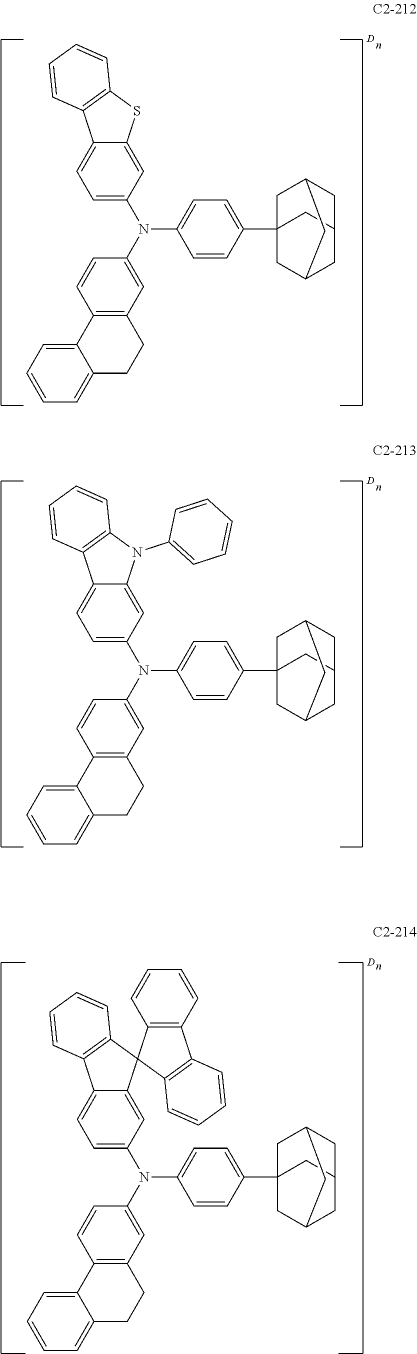

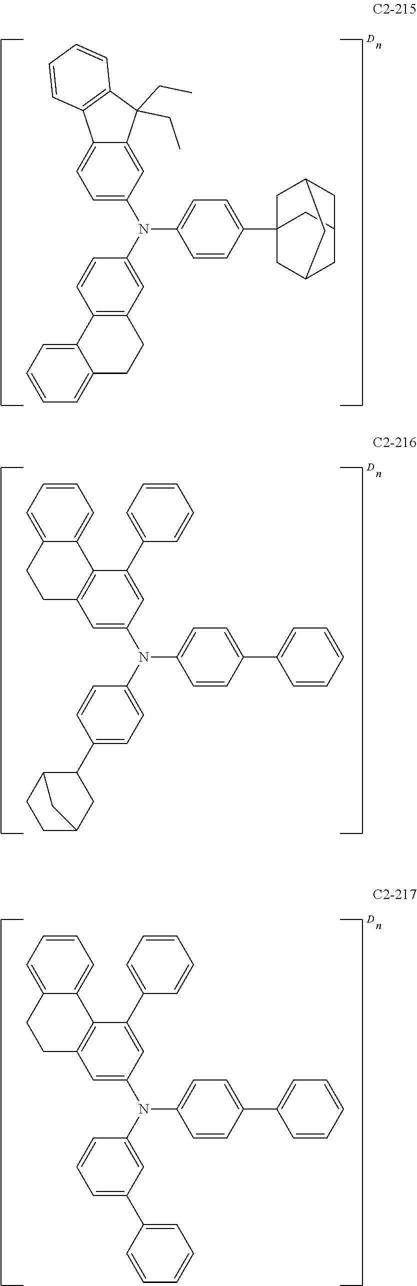

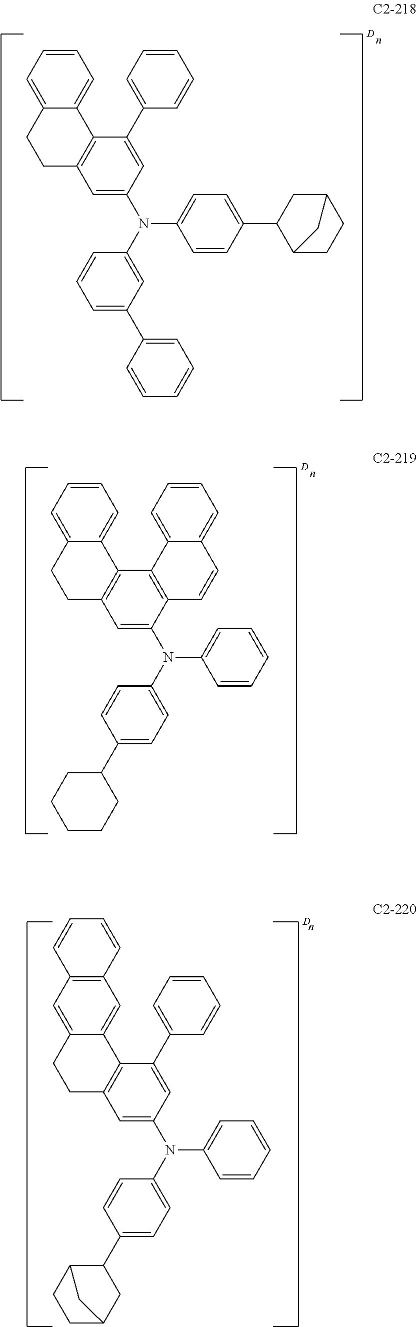

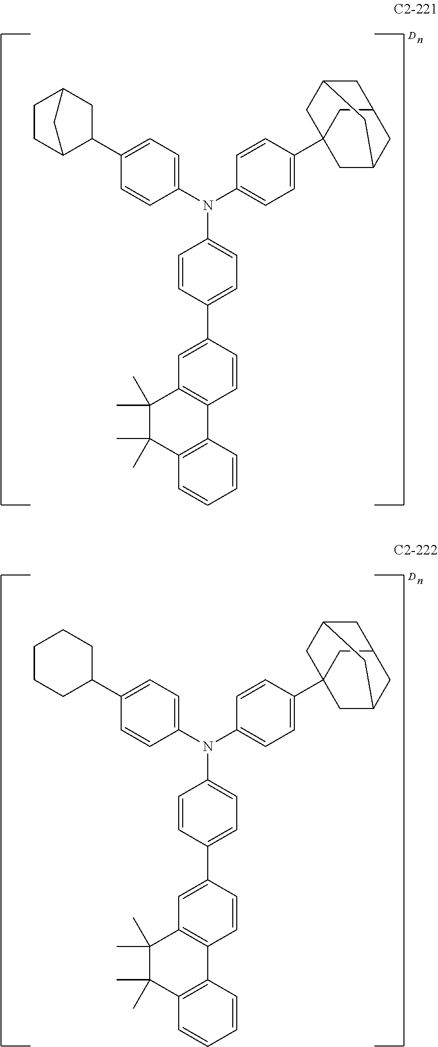

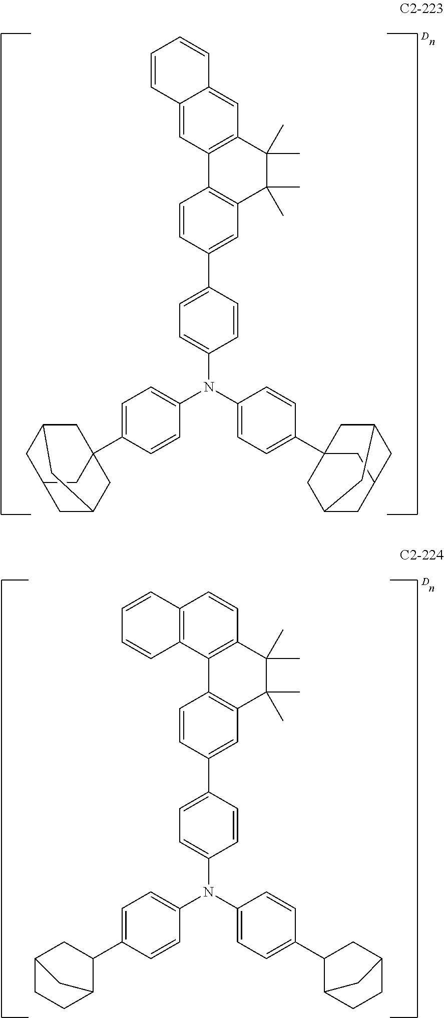

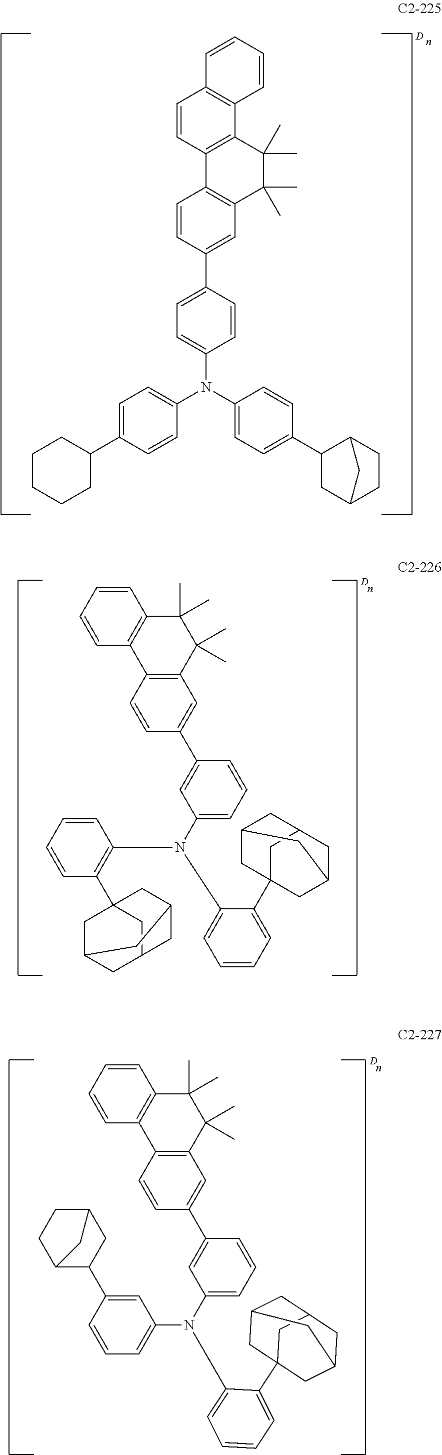

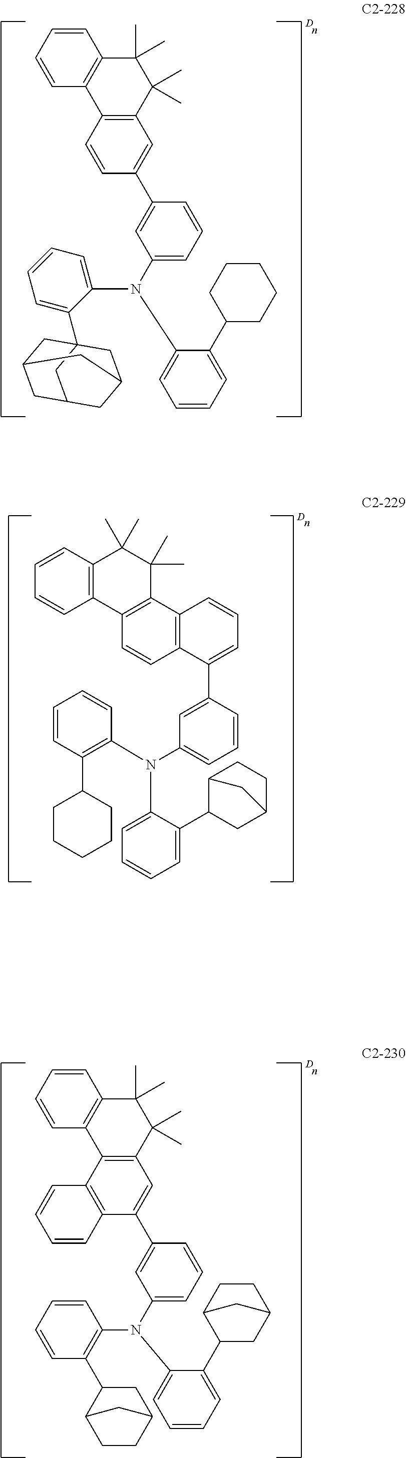

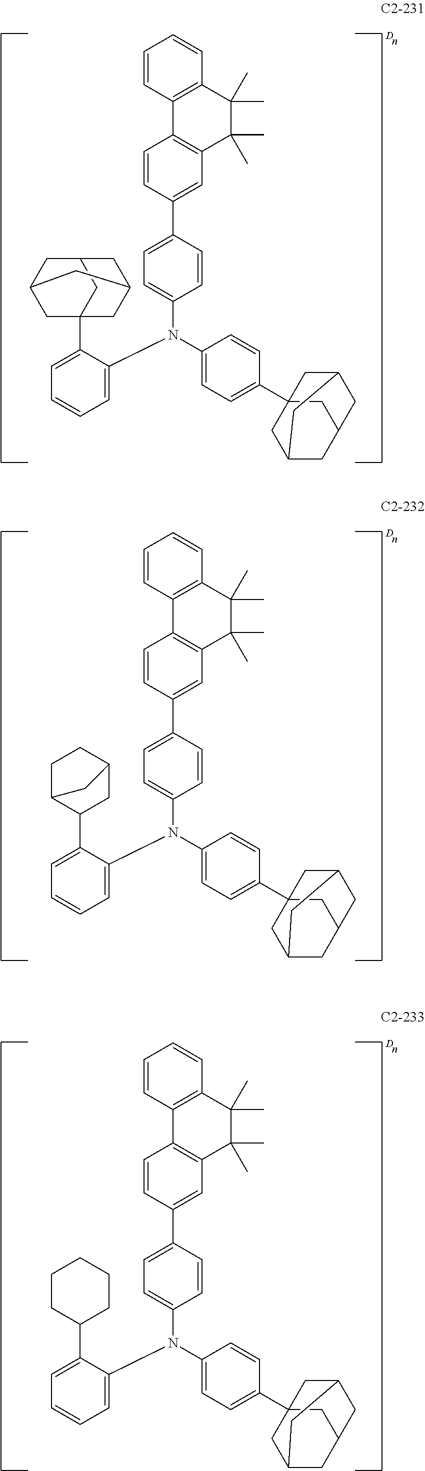

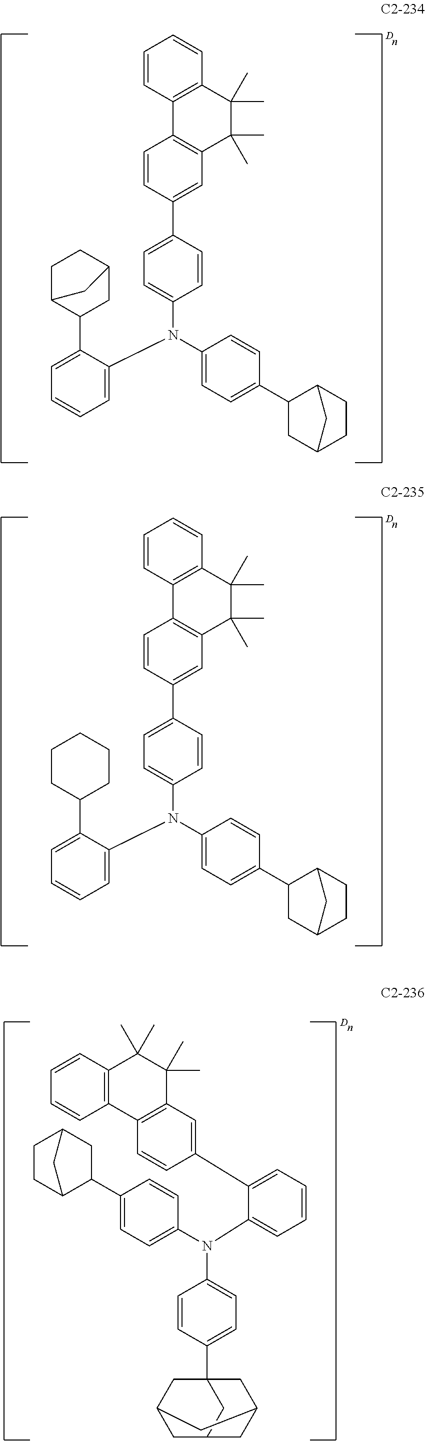

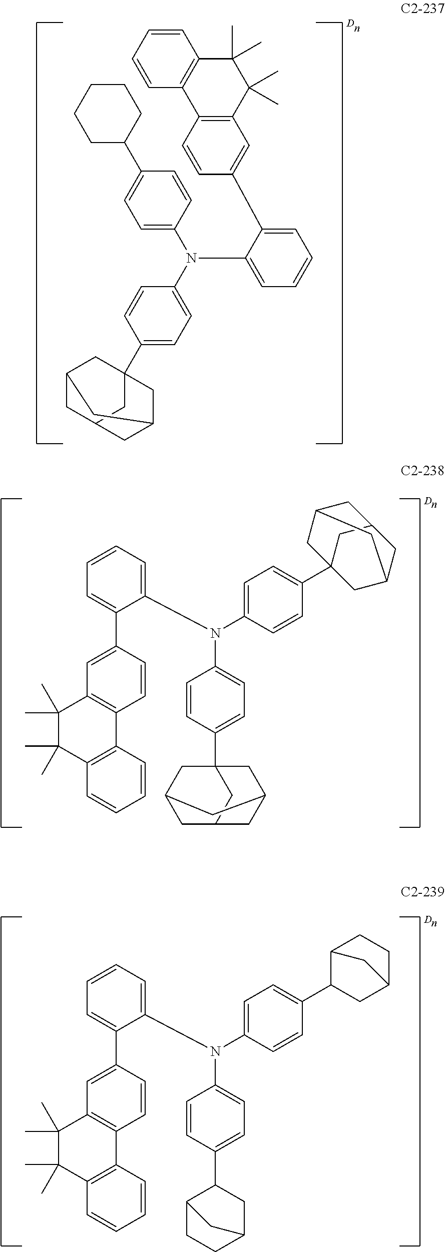

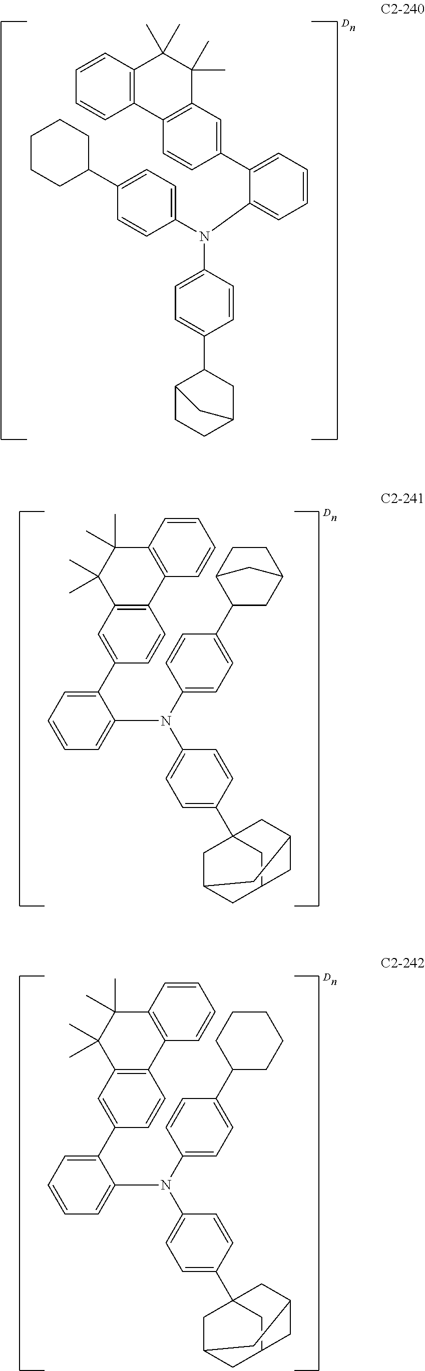

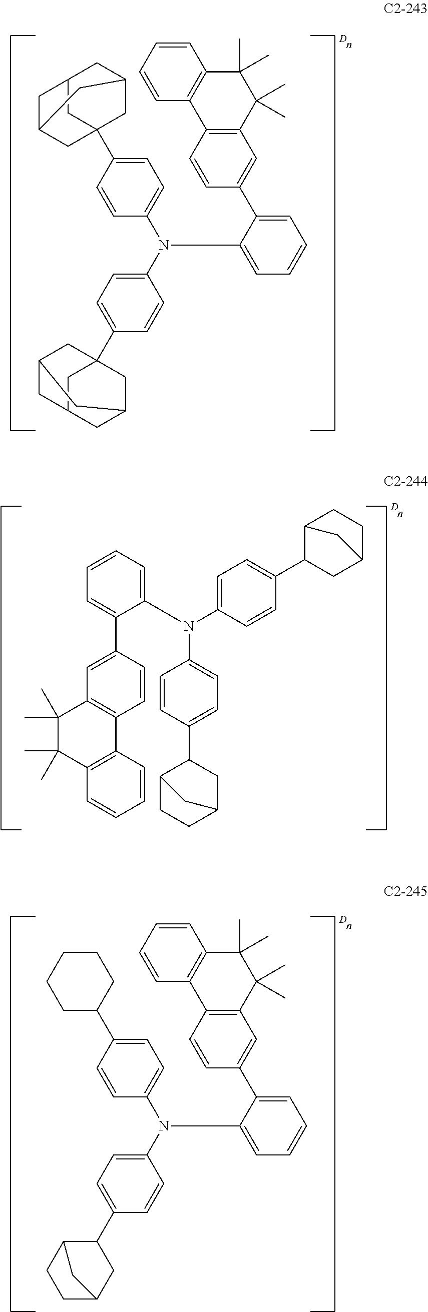

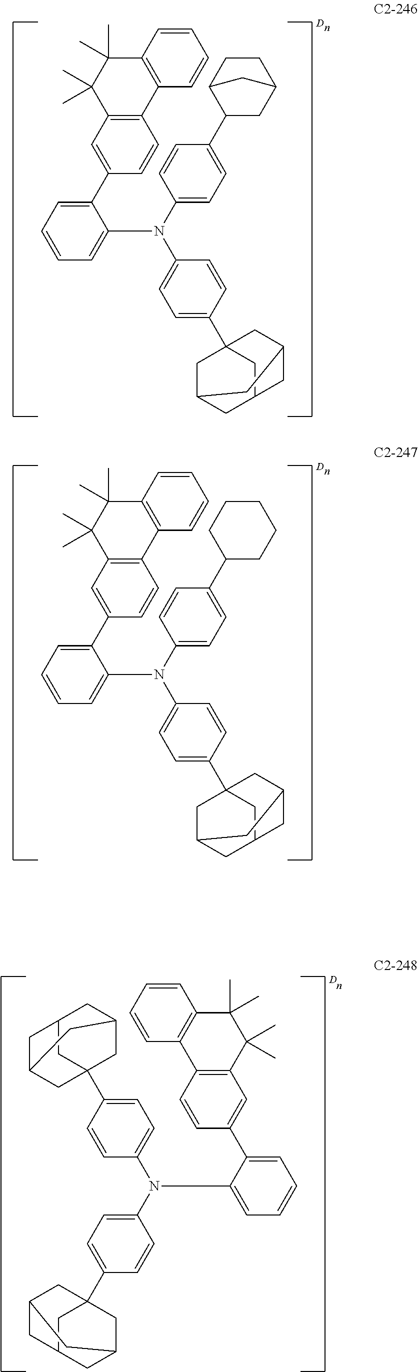

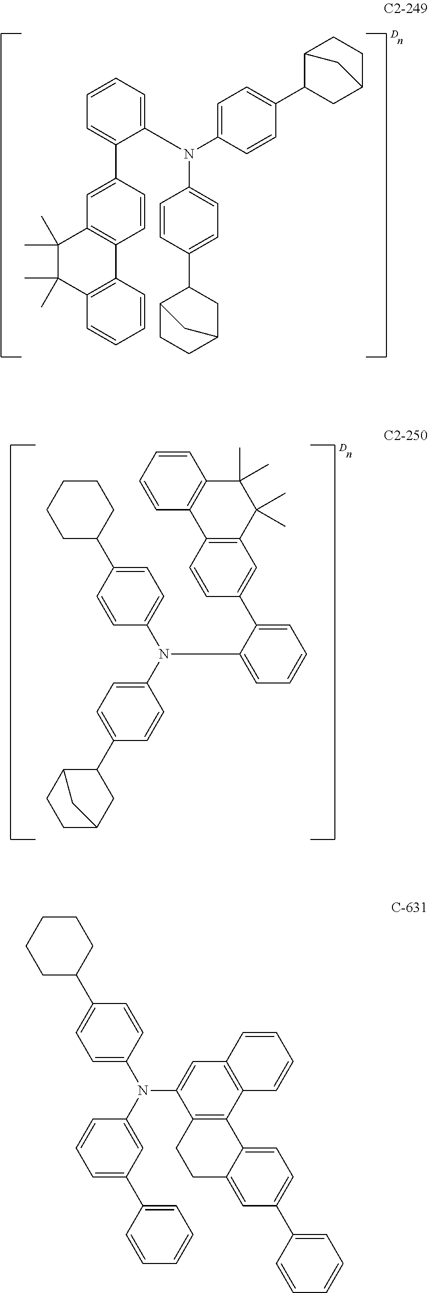

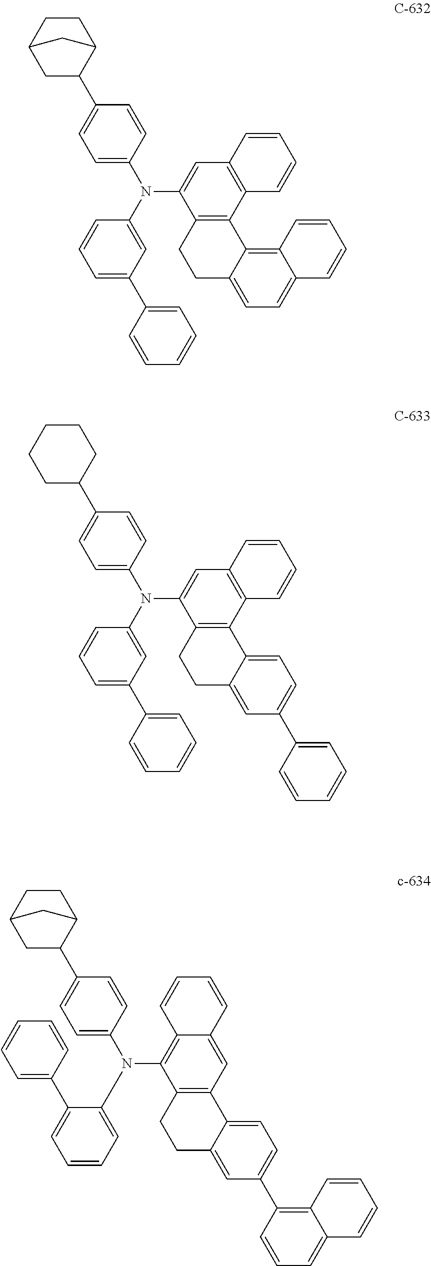 [SMILES: c1ccc(-c2cccc(N(c3ccc(C4CC5CCC4C5)cc3)c3cc4ccccc4c4c3CCc3ccc5ccccc5c3-4)c2)cc1 ; c1ccc(-c2cccc(N(c3ccc(C4CCCCC4)cc3)c3cc4ccccc4c4c3CCc3cc(-c5ccccc5)ccc3-4)c2)cc1 ; c1ccc(-c2ccccc2N(c2ccc(C3CC4CCC3C4)cc2)c2c3c(cc4ccccc24)-c2ccc(-c4cccc5ccccc45)cc2CC3)cc1]

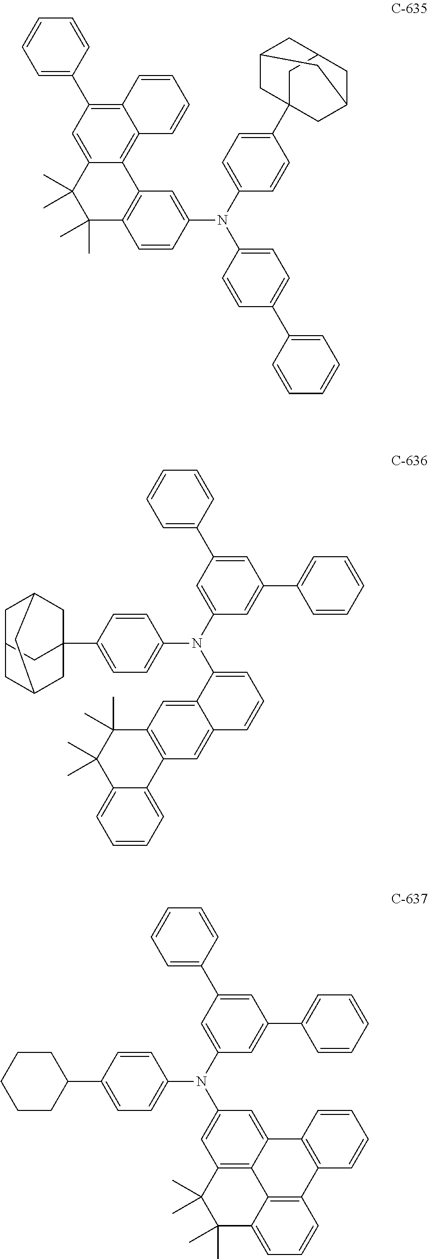 [SMILES: CC1(C)c2ccc(N(c3ccc(-c4ccccc4)cc3)c3ccc(C45CC6CC(CC(C6)C4)C5)cc3)cc2-c2c(cc(-c3ccccc3)c3ccccc23)C1(C)C ; CC1(C)c2ccccc2-c2cc3cccc(N(c4ccc(C56CC7CC(CC(C7)C5)C6)cc4)c4cc(-c5ccccc5)cc(-c5ccccc5)c4)c3cc2C1(C)C ; CC1(C)c2cccc3c4ccccc4c4cc(N(c5ccc(C6CCCCC6)cc5)c5cc(-c6ccccc6)cc(-c6ccccc6)c5)cc(c4c23)C1(C)C]

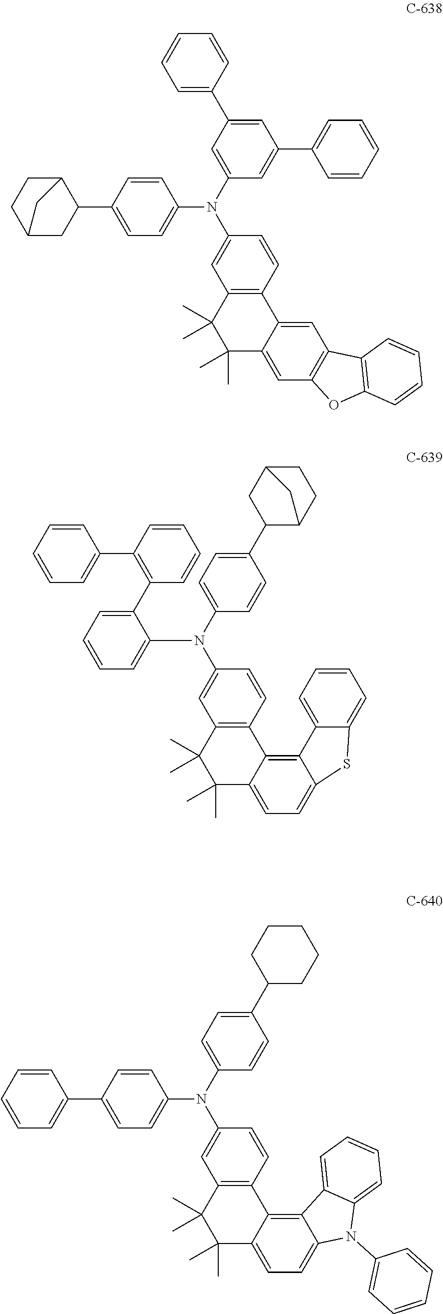 [SMILES: CC1(C)c2cc(N(c3ccc(C4CC5CCC4C5)cc3)c3cc(-c4ccccc4)cc(-c4ccccc4)c3)ccc2-c2cc3c(cc2C1(C)C)oc1ccccc13 ; CC1(C)c2cc(N(c3ccc(C4CC5CCC4C5)cc3)c3ccccc3-c3ccccc3-c3ccccc3)ccc2-c2c(ccc3sc4ccccc4c23)C1(C)C ; CC1(C)c2cc(N(c3ccc(-c4ccccc4)cc3)c3ccc(C4CCCCC4)cc3)ccc2-c2c(ccc3c2c2ccccc2n3-c2ccccc2)C1(C)C]

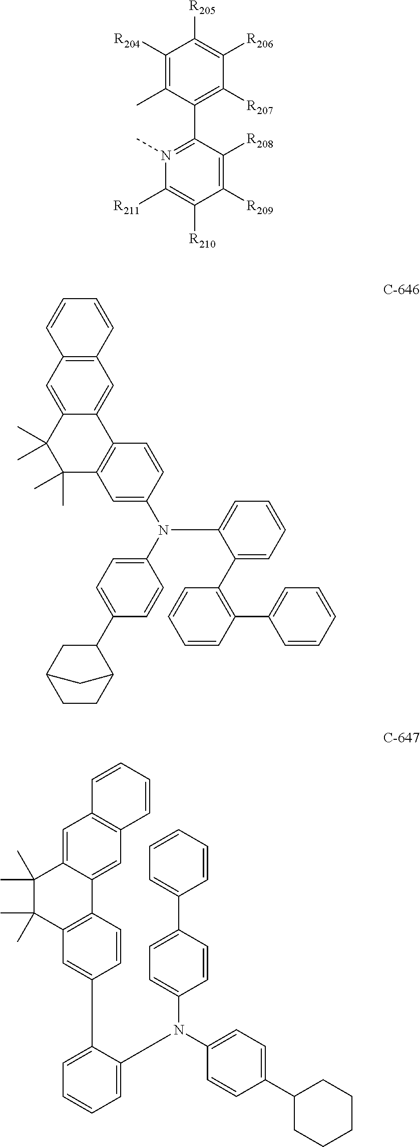

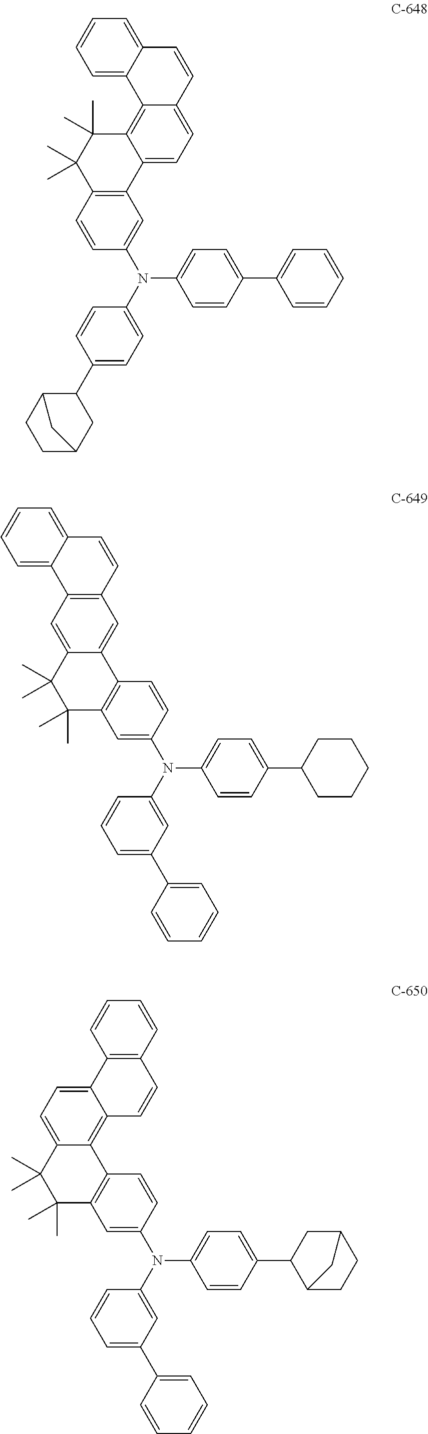 [SMILES: CC1(C)c2ccc(N(c3ccc(-c4ccccc4)cc3)c3ccc(C4CC5CCC4C5)cc3)cc2-c2ccc3ccc4ccccc4c3c2C1(C)C ; CC1(C)c2cc(N(c3ccc(C4CCCCC4)cc3)c3cccc(-c4ccccc4)c3)ccc2-c2cc3ccc4ccccc4c3cc2C1(C)C ; CC1(C)c2cc(N(c3ccc(C4CC5CCC4C5)cc3)c3cccc(-c4ccccc4)c3)ccc2-c2c(ccc3c2ccc2ccccc23)C1(C)C]

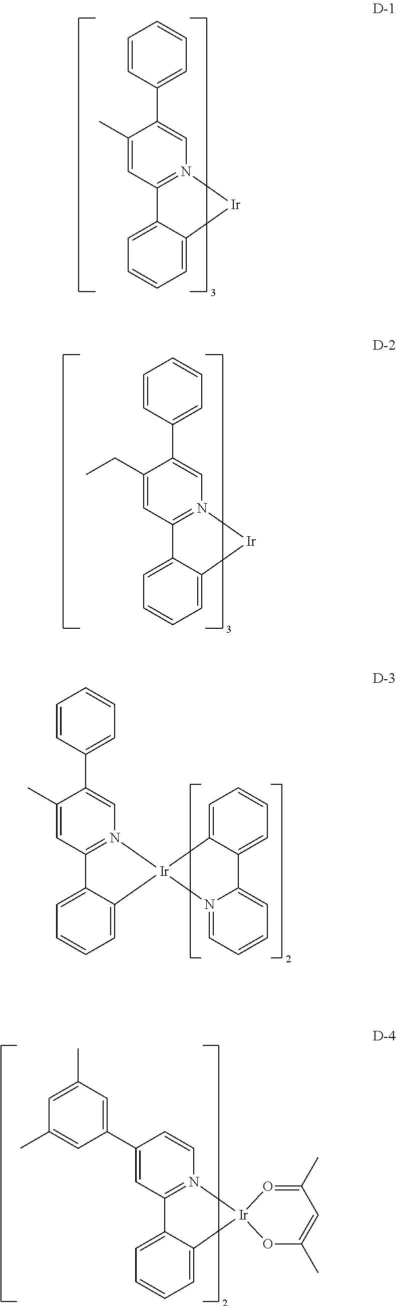

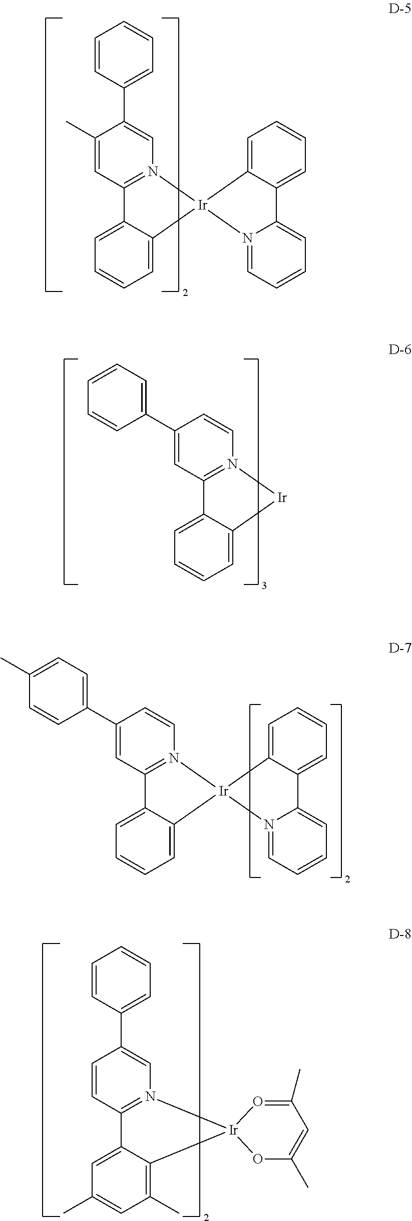

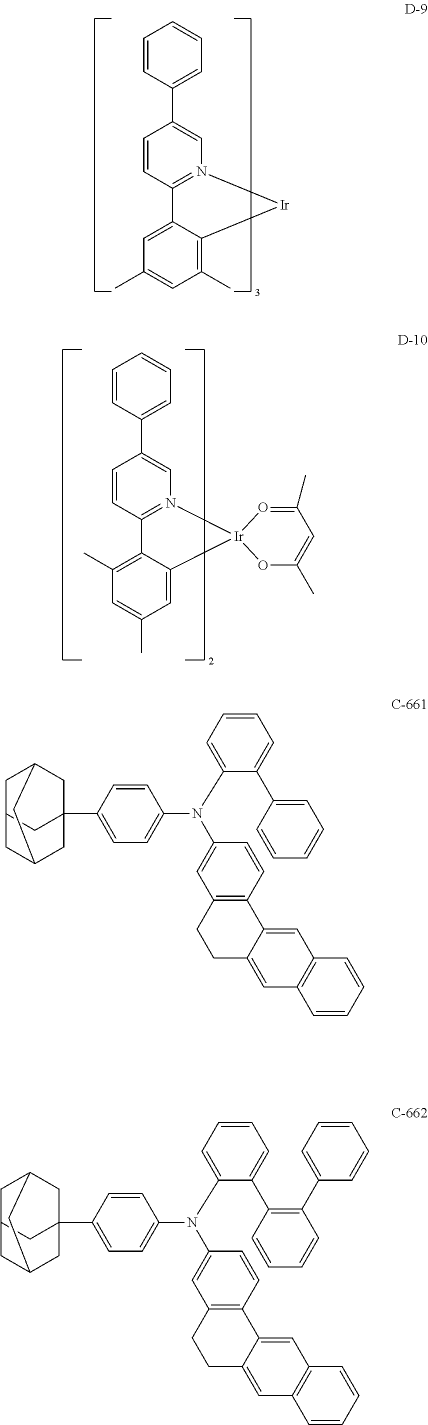

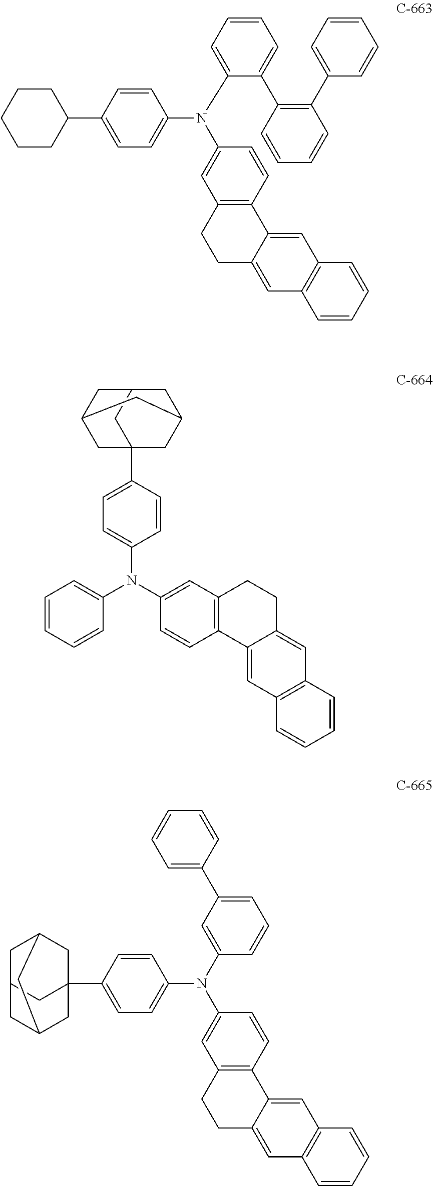 [SMILES: c1ccc(-c2ccccc2-c2ccccc2N(c2ccc(C3CCCCC3)cc2)c2ccc3c(c2)CCc2cc4ccccc4cc2-3)cc1 ; c1ccc(N(c2ccc(C34CC5CC(CC(C5)C3)C4)cc2)c2ccc3c(c2)CCc2cc4ccccc4cc2-3)cc1 ; c1ccc(-c2cccc(N(c3ccc(C45CC6CC(CC(C6)C4)C5)cc3)c3ccc4c(c3)CCc3cc5ccccc5cc3-4)c2)cc1]

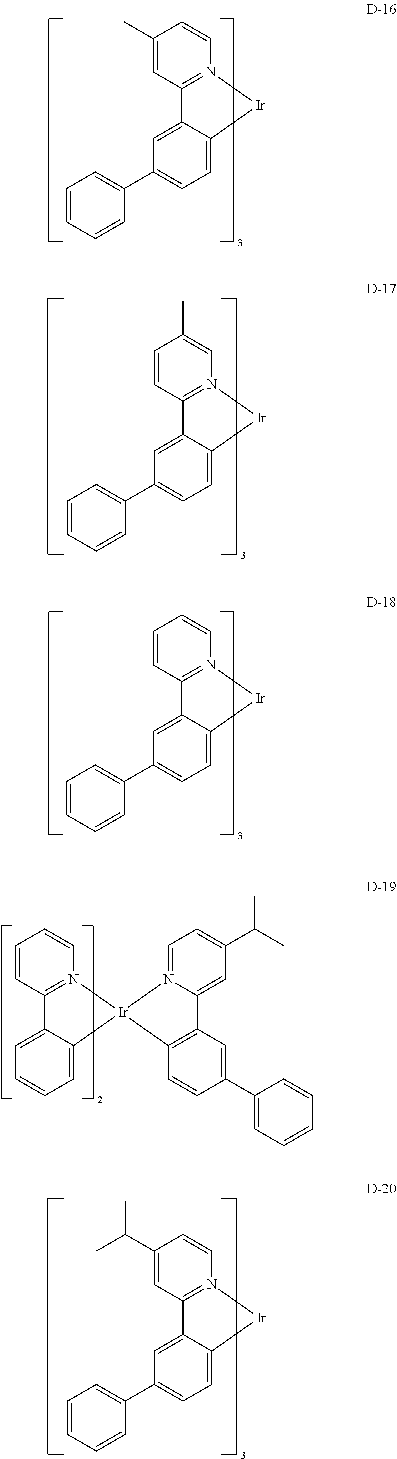

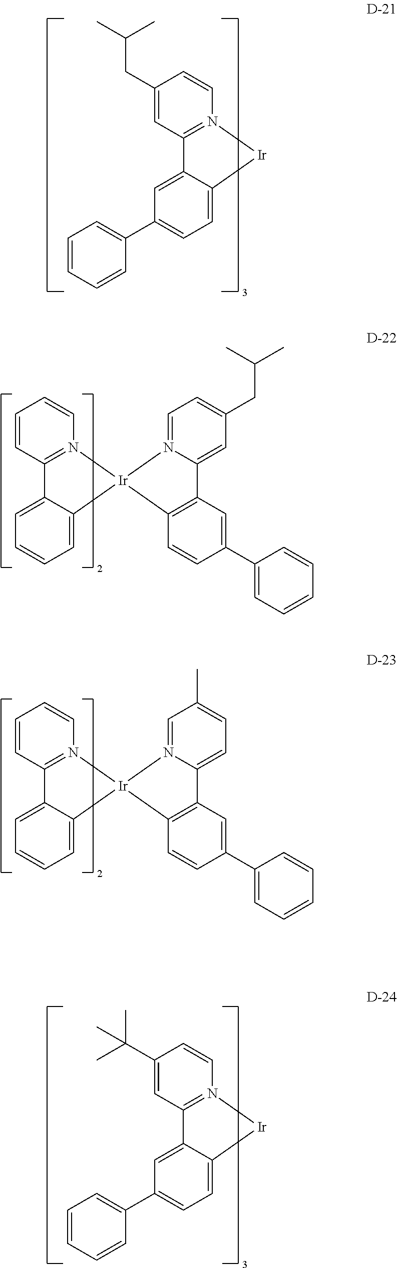

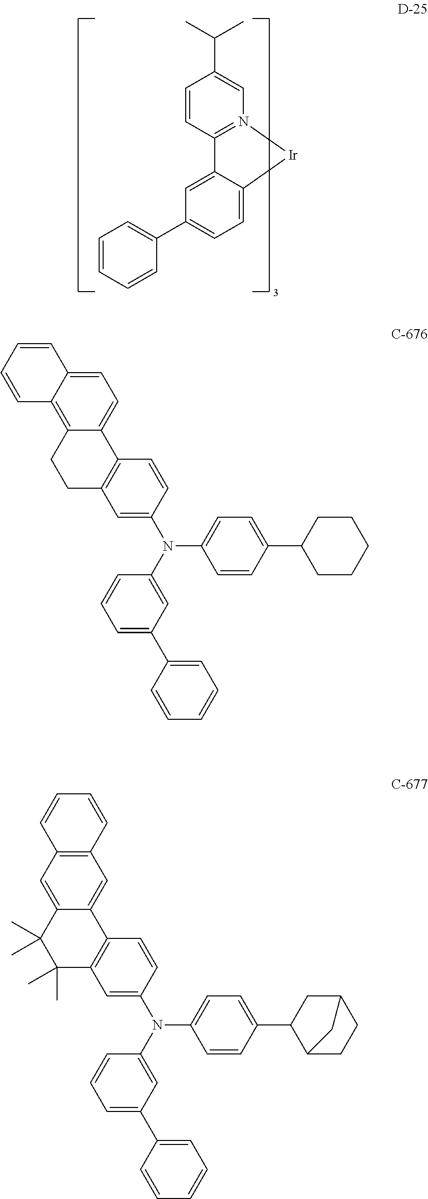

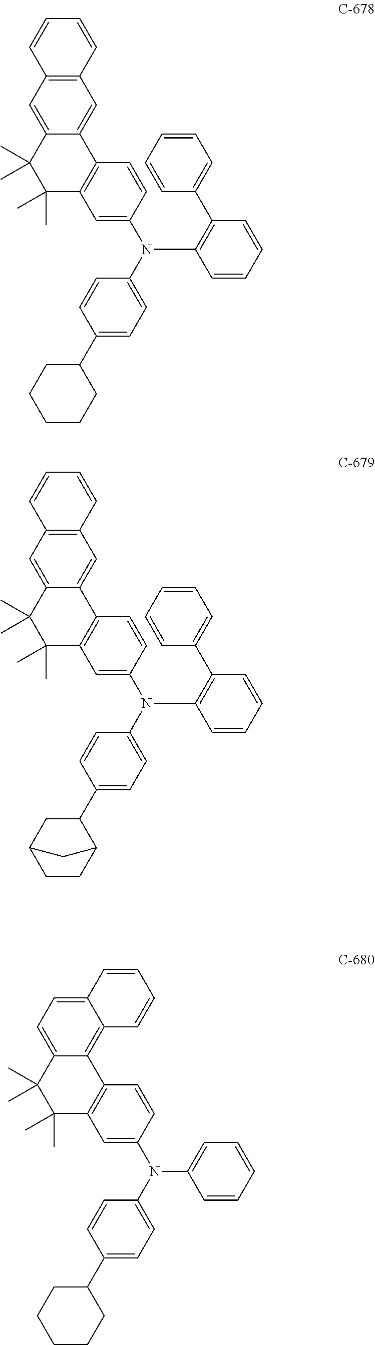 [SMILES: CC1(C)c2cc(N(c3ccc(C4CCCCC4)cc3)c3ccccc3-c3ccccc3)ccc2-c2cc3ccccc3cc2C1(C)C ; CC1(C)c2cc(N(c3ccc(C4CC5CCC4C5)cc3)c3ccccc3-c3ccccc3)ccc2-c2cc3ccccc3cc2C1(C)C ; CC1(C)c2cc(N(c3ccccc3)c3ccc(C4CCCCC4)cc3)ccc2-c2c(ccc3ccccc23)C1(C)C]

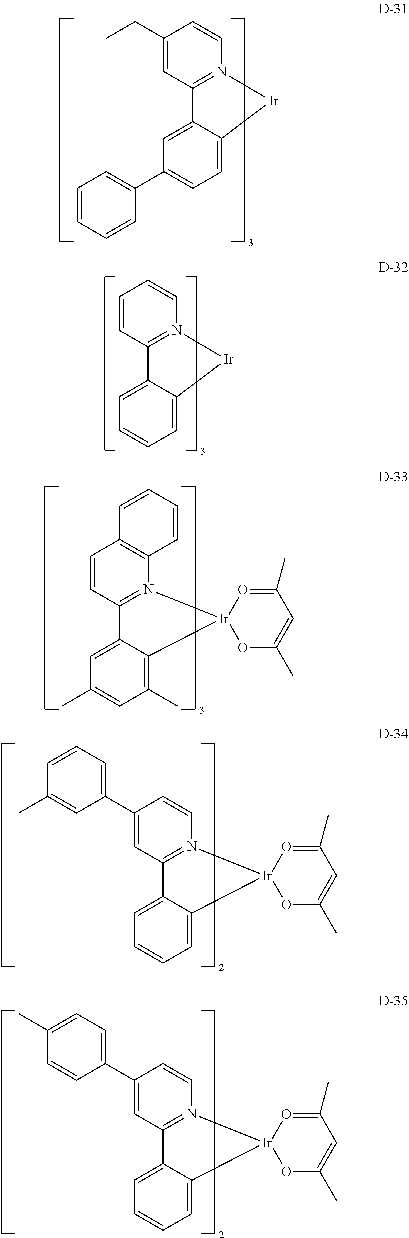

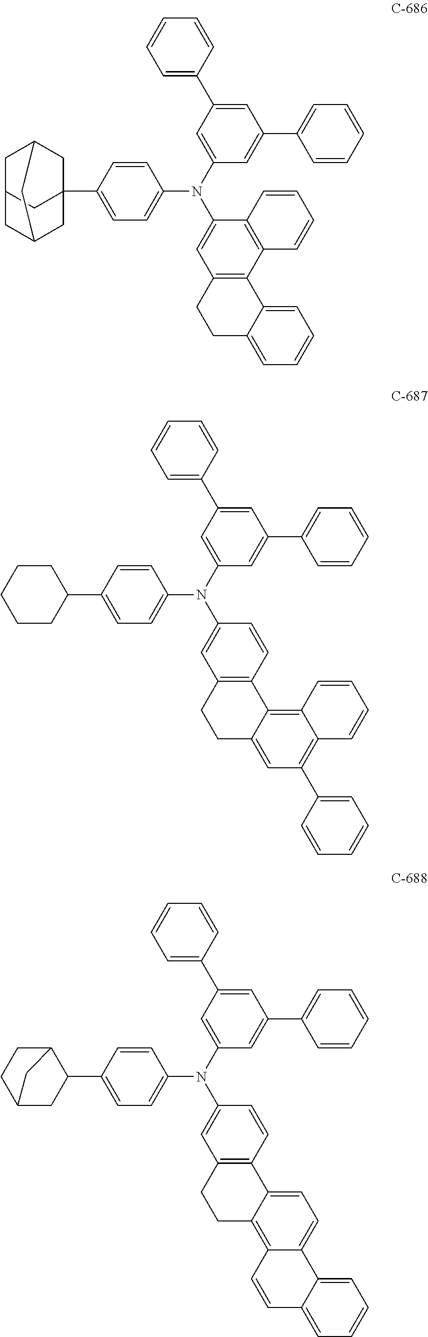 [SMILES: c1ccc(-c2cc(-c3ccccc3)cc(N(c3ccc(C45CC6CC(CC(C6)C4)C5)cc3)c3cc4c(c5ccccc35)-c3ccccc3CC4)c2)cc1 ; c1ccc(-c2cc(-c3ccccc3)cc(N(c3ccc(C4CCCCC4)cc3)c3ccc4c(c3)CCc3cc(-c5ccccc5)c5ccccc5c3-4)c2)cc1 ; c1ccc(-c2cc(-c3ccccc3)cc(N(c3ccc(C4CC5CCC4C5)cc3)c3ccc4c(c3)CCc3c-4ccc4c3ccc3ccccc34)c2)cc1]

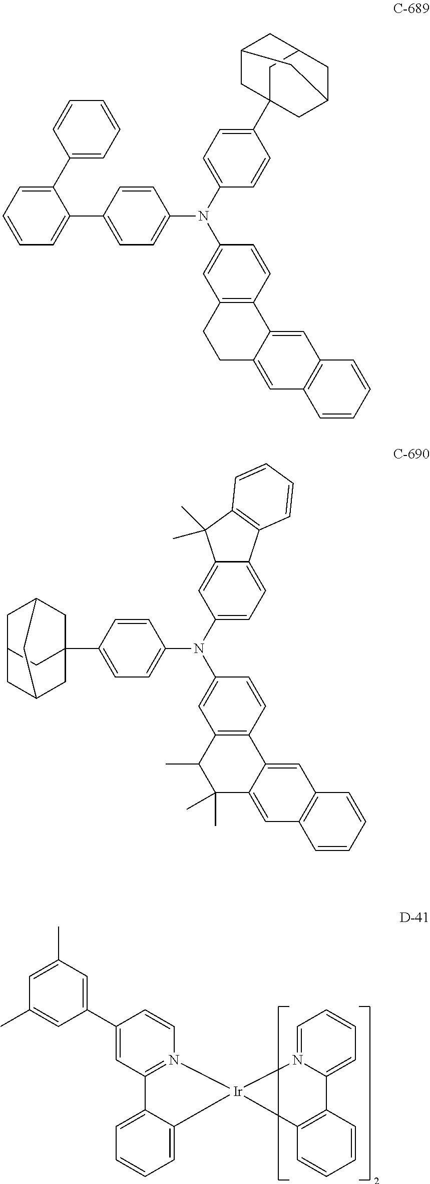

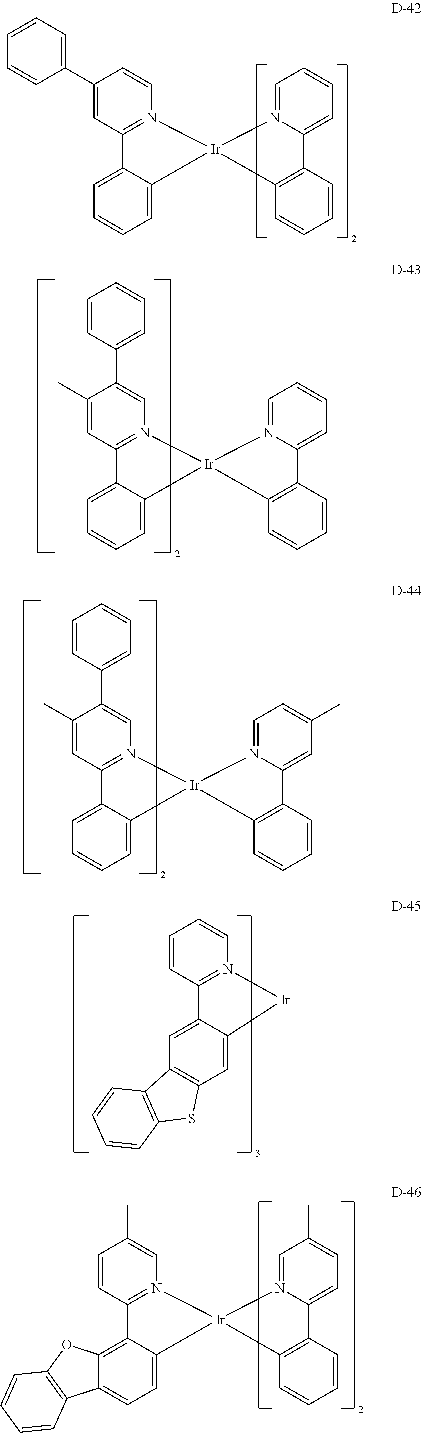

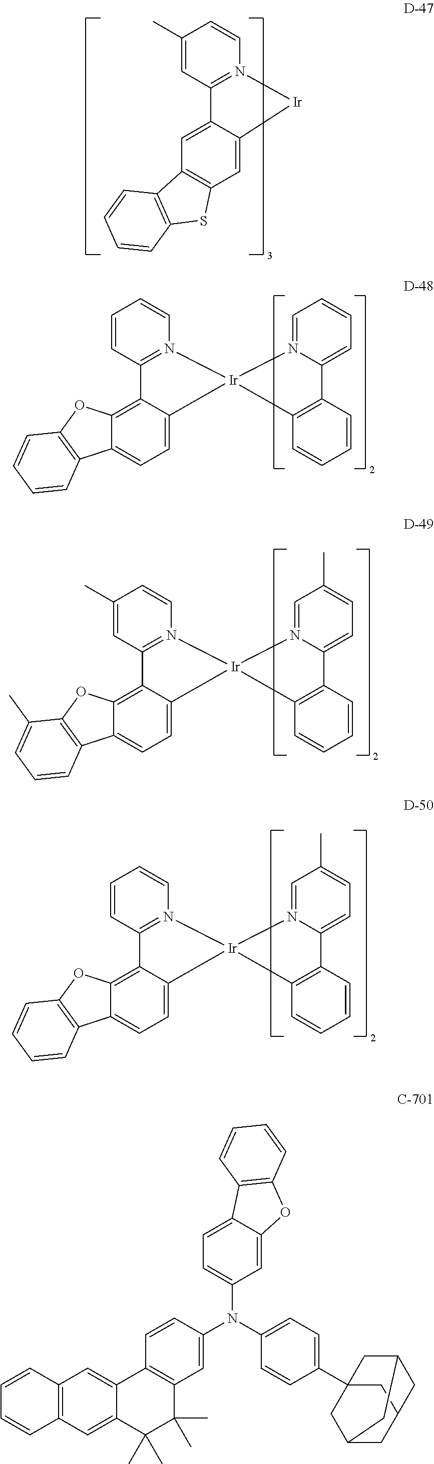

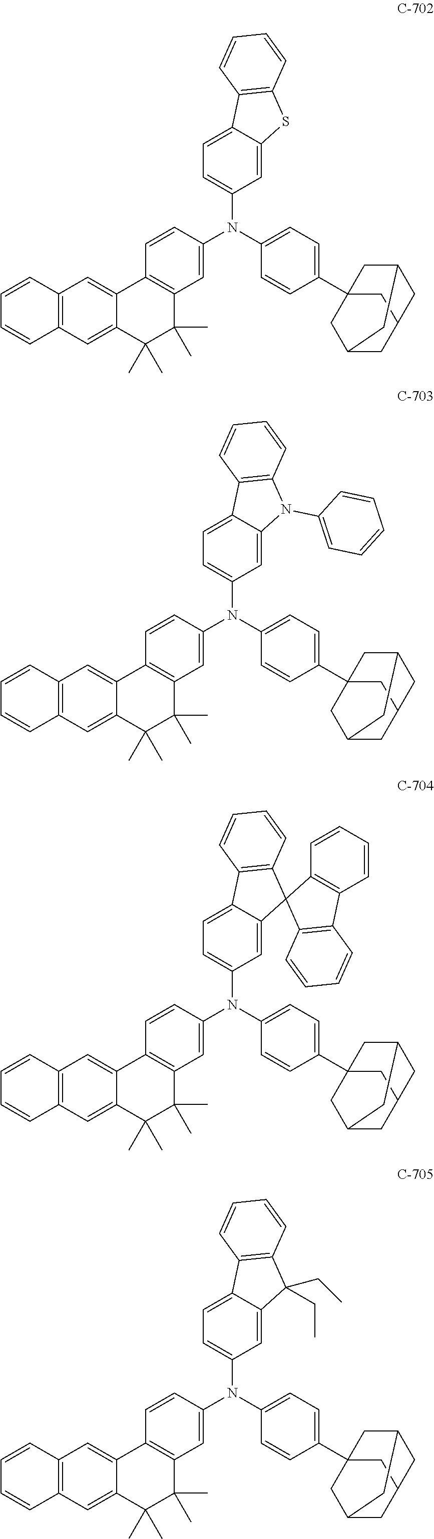 [SMILES: CC1(C)c2cc(N(c3ccc(C45CC6CC(CC(C6)C4)C5)cc3)c3ccc4c(c3)sc3ccccc34)ccc2-c2cc3ccccc3cc2C1(C)C ; CC1(C)c2cc(N(c3ccc(C45CC6CC(CC(C6)C4)C5)cc3)c3ccc4c5ccccc5n(-c5ccccc5)c4c3)ccc2-c2cc3ccccc3cc2C1(C)C ; CC1(C)c2cc(N(c3ccc(C45CC6CC(CC(C6)C4)C5)cc3)c3ccc4c(c3)C3(c5ccccc5-c5ccccc53)c3ccccc3-4)ccc2-c2cc3ccccc3cc2C1(C)C ; CCC1(CC)c2ccccc2-c2ccc(N(c3ccc(C45CC6CC(CC(C6)C4)C5)cc3)c3ccc4c(c3)C(C)(C)C(C)(C)c3cc5ccccc5cc3-4)cc21]

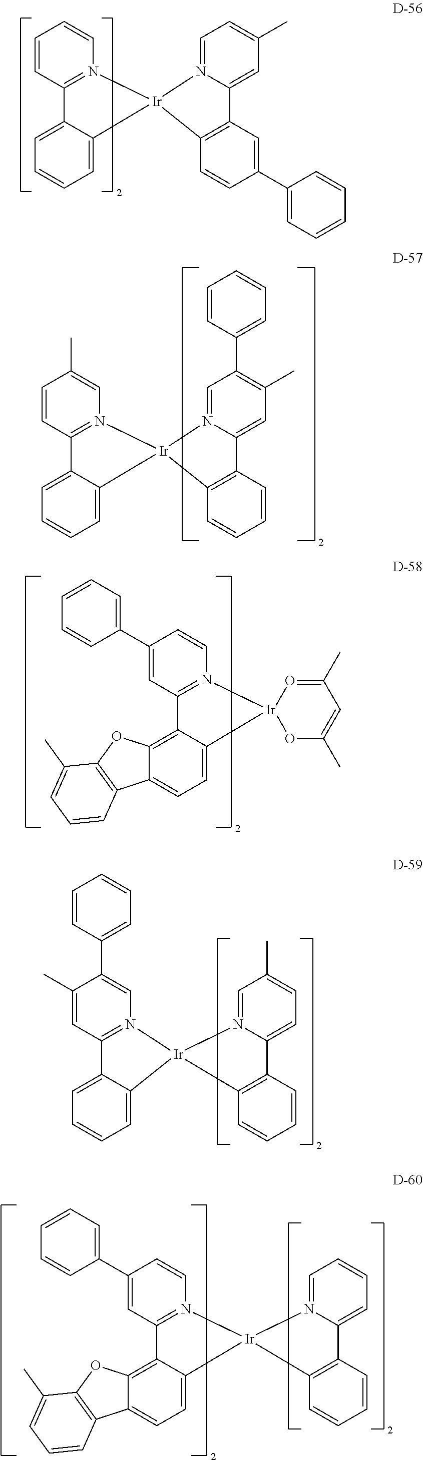

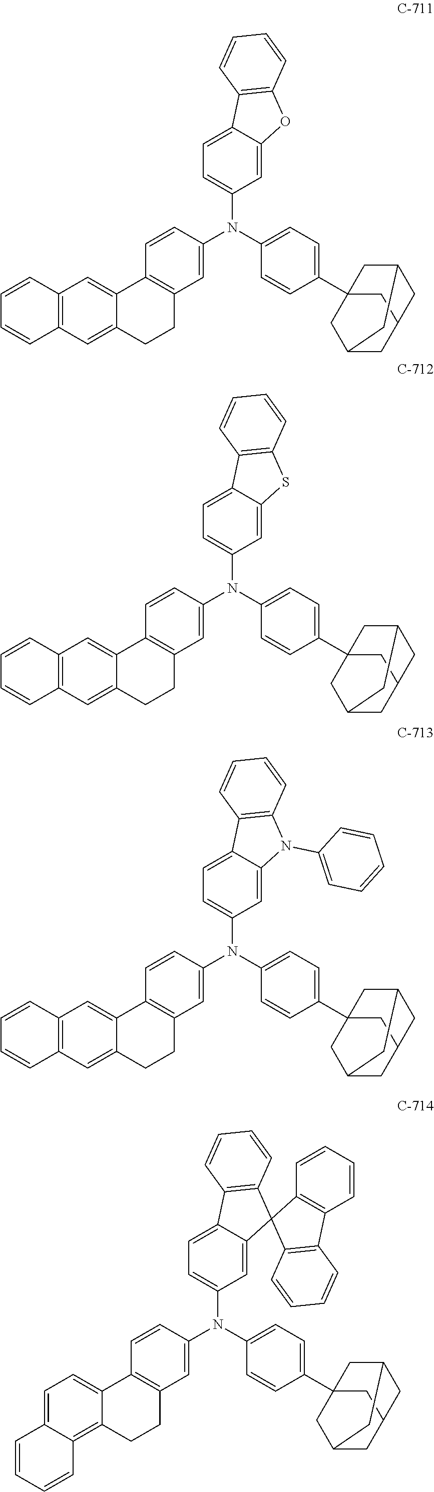 [SMILES: c1ccc2cc3c(cc2c1)CCc1cc(N(c2ccc(C45CC6CC(CC(C6)C4)C5)cc2)c2ccc4c(c2)oc2ccccc24)ccc1-3 ; c1ccc2cc3c(cc2c1)CCc1cc(N(c2ccc(C45CC6CC(CC(C6)C4)C5)cc2)c2ccc4c(c2)sc2ccccc24)ccc1-3 ; c1ccc(-n2c3ccccc3c3ccc(N(c4ccc(C56CC7CC(CC(C7)C5)C6)cc4)c4ccc5c(c4)CCc4cc6ccccc6cc4-5)cc32)cc1 ; c1ccc2c(c1)-c1ccccc1C21c2ccccc2-c2ccc(N(c3ccc(C45CC6CC(CC(C6)C4)C5)cc3)c3ccc4c(c3)CCc3c-4ccc4ccccc34)cc21]

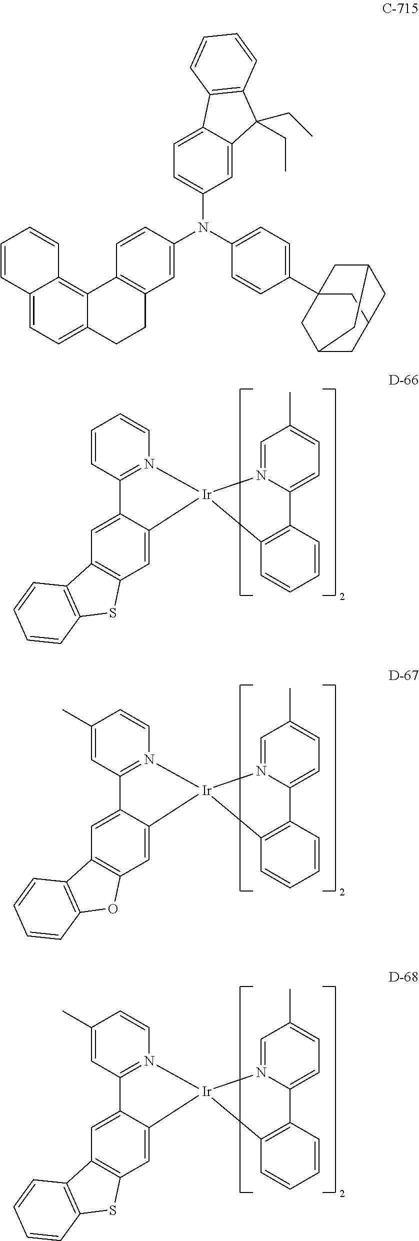

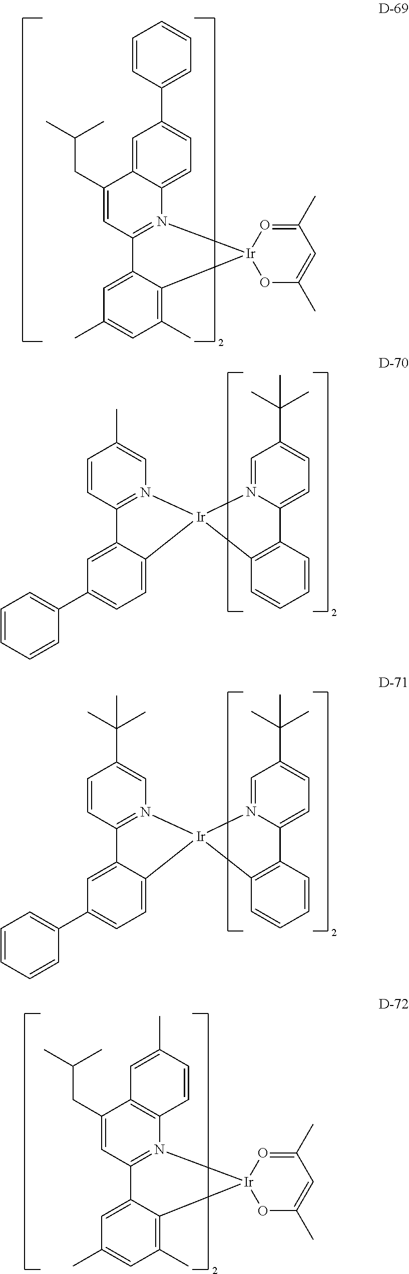

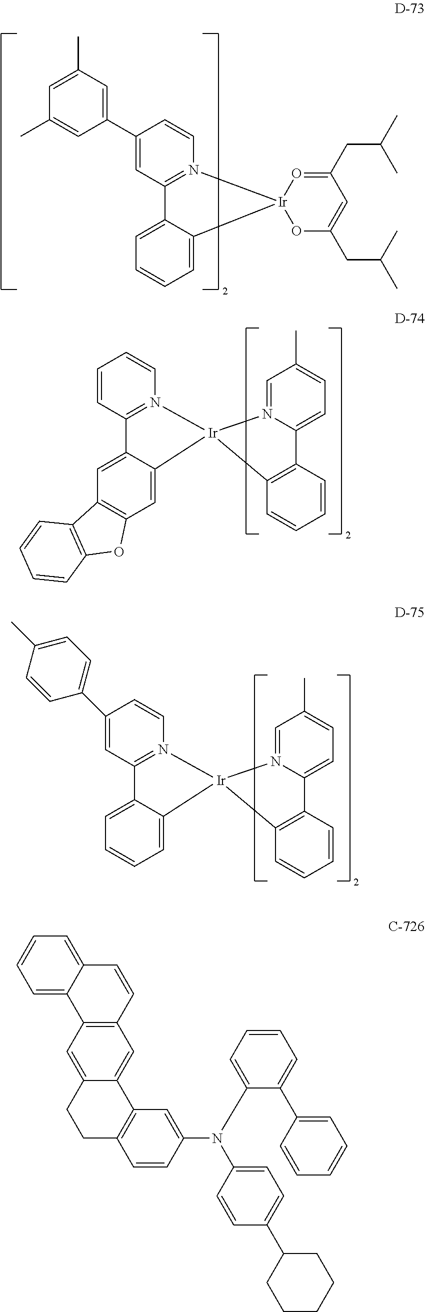

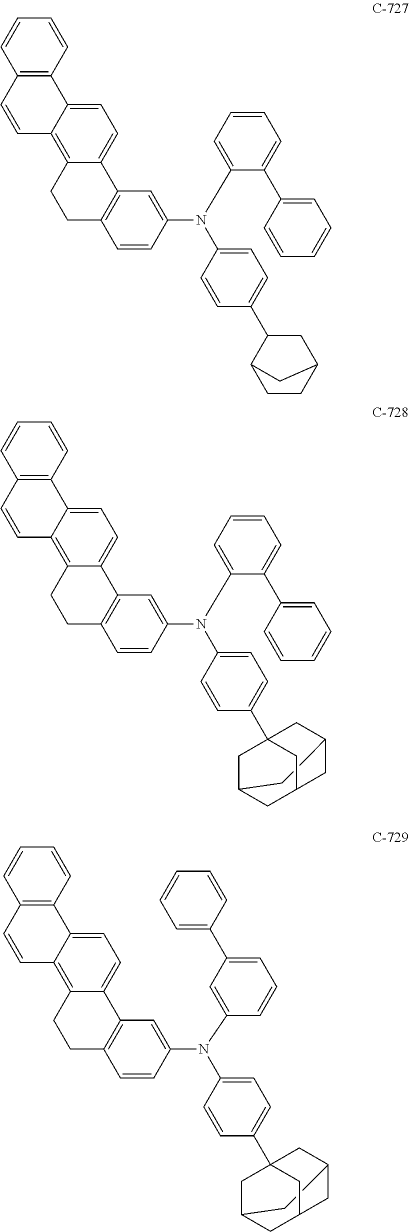 [SMILES: c1ccc(-c2ccccc2N(c2ccc(C3CC4CCC3C4)cc2)c2ccc3c(c2)-c2ccc4c(ccc5ccccc54)c2CC3)cc1 ; c1ccc(-c2ccccc2N(c2ccc(C34CC5CC(CC(C5)C3)C4)cc2)c2ccc3c(c2)-c2ccc4c(ccc5ccccc54)c2CC3)cc1 ; c1ccc(-c2cccc(N(c3ccc(C45CC6CC(CC(C6)C4)C5)cc3)c3ccc4c(c3)-c3ccc5c(ccc6ccccc65)c3CC4)c2)cc1]

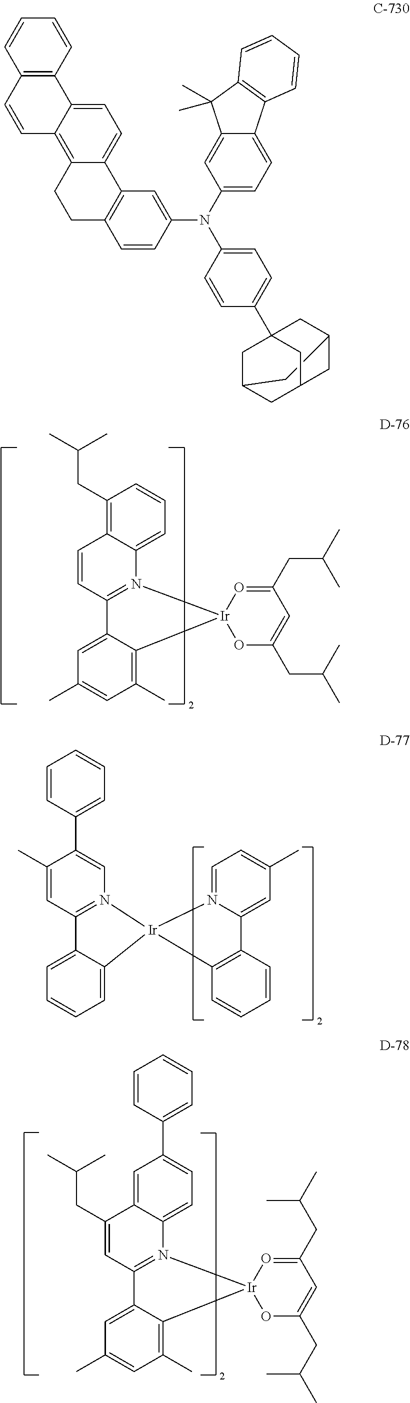

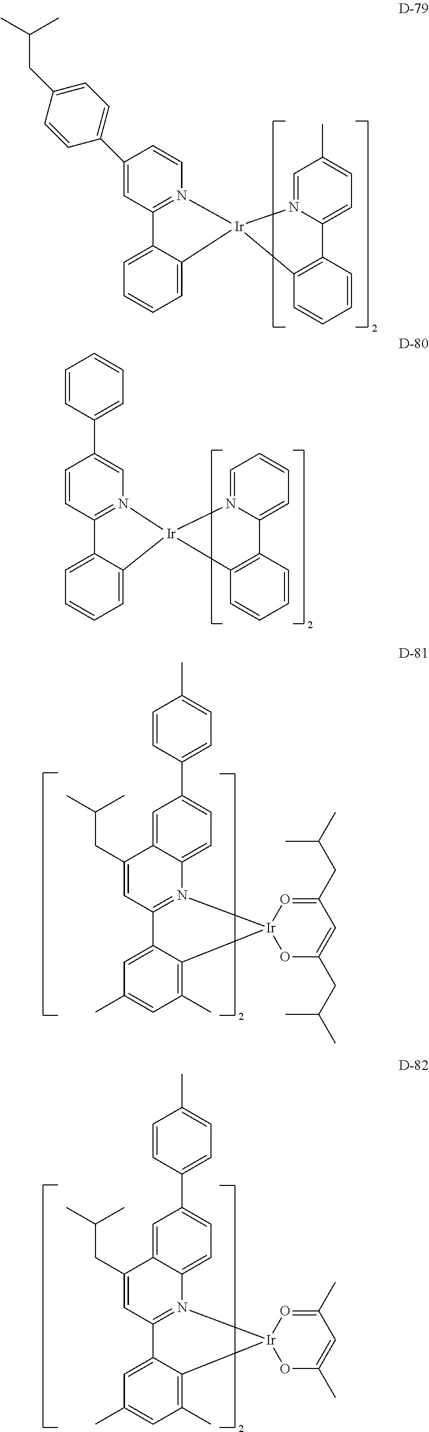

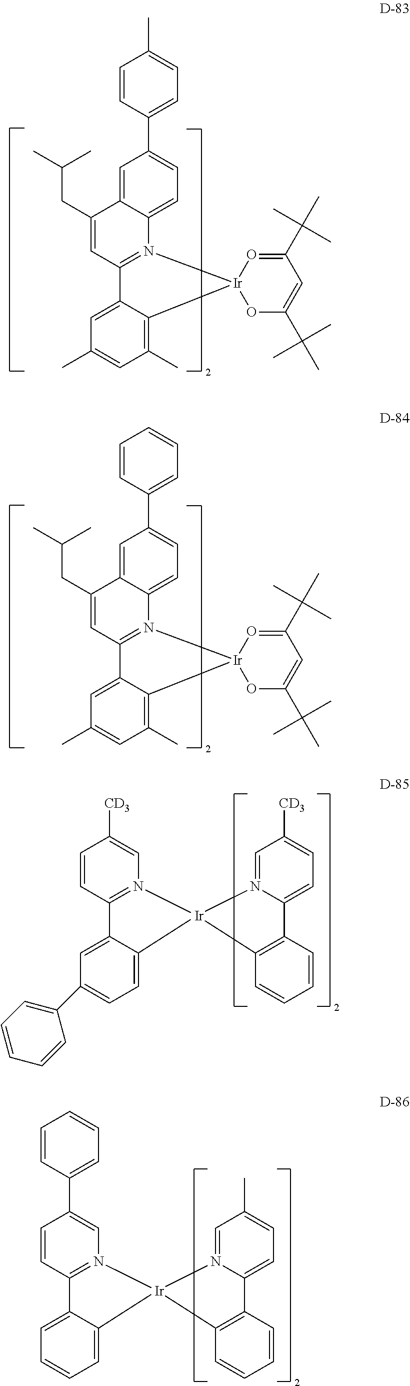

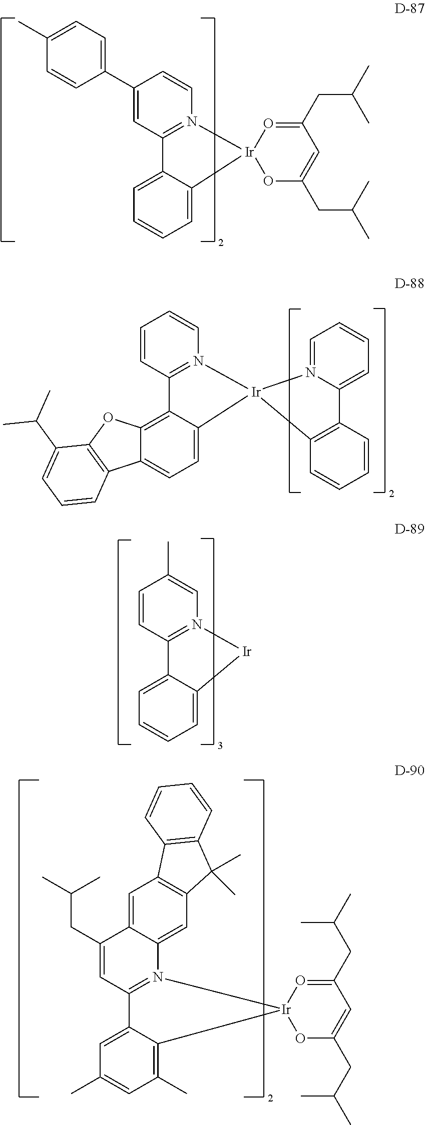

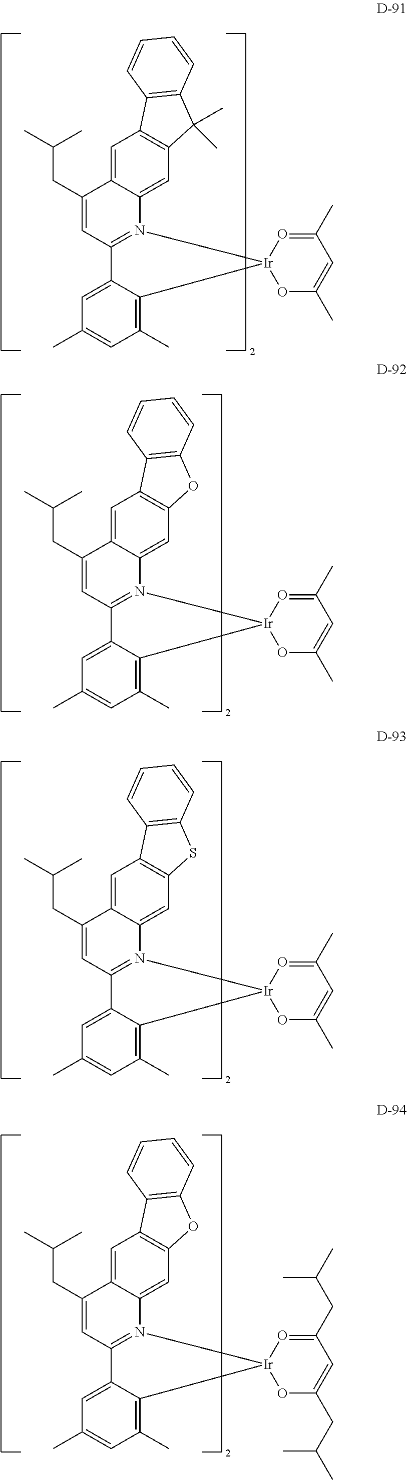

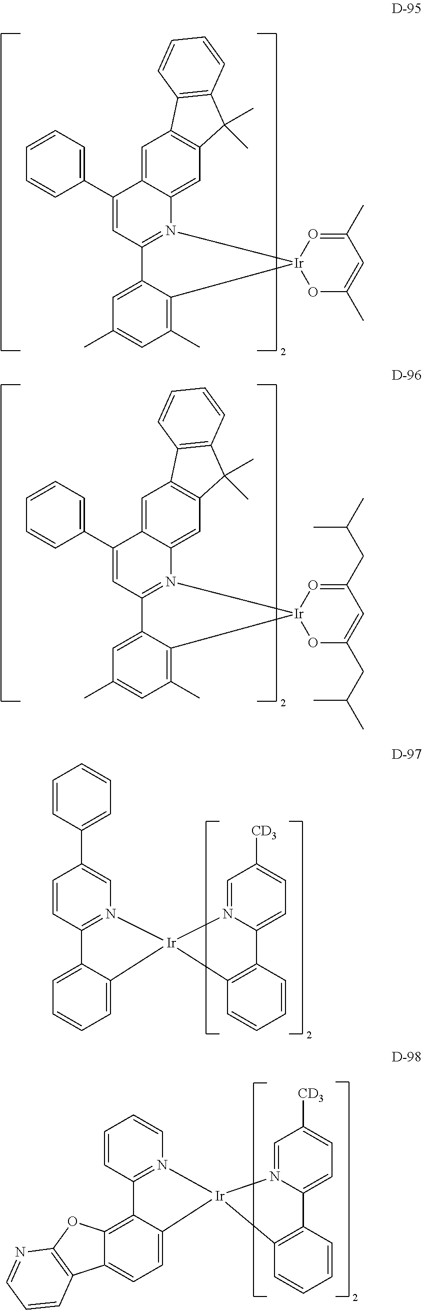

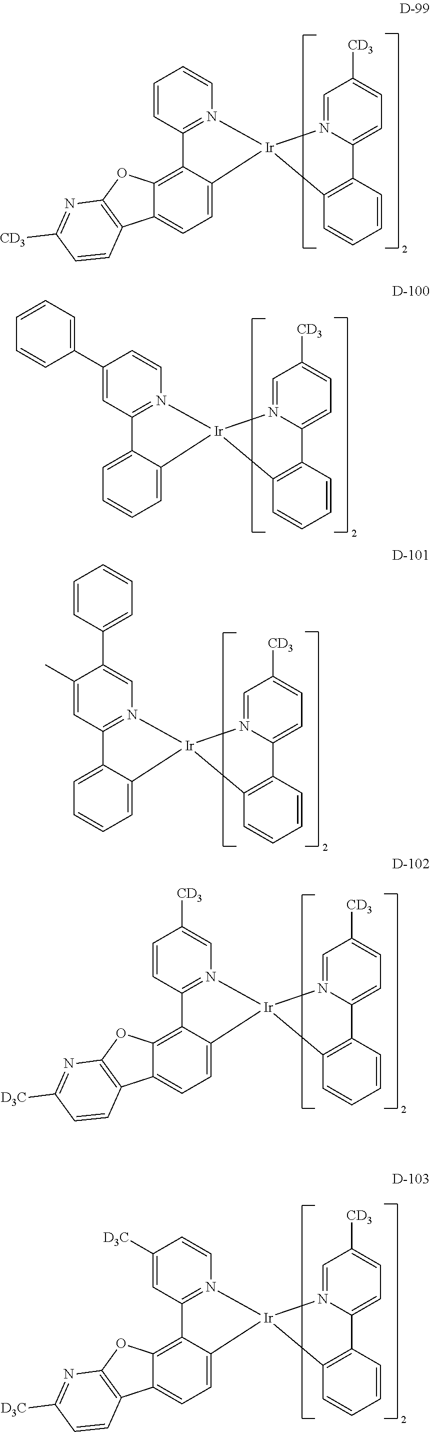

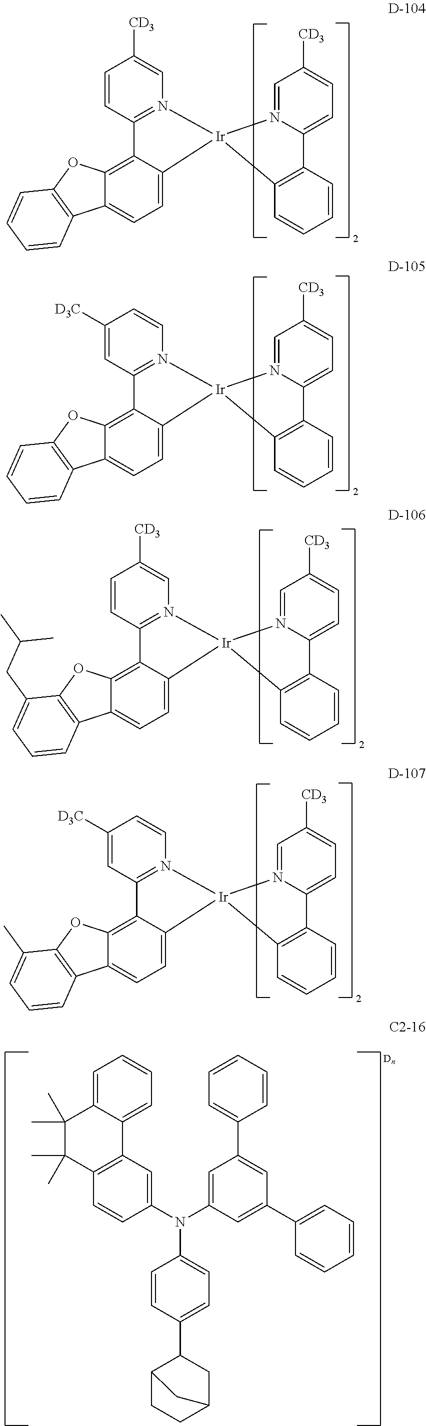

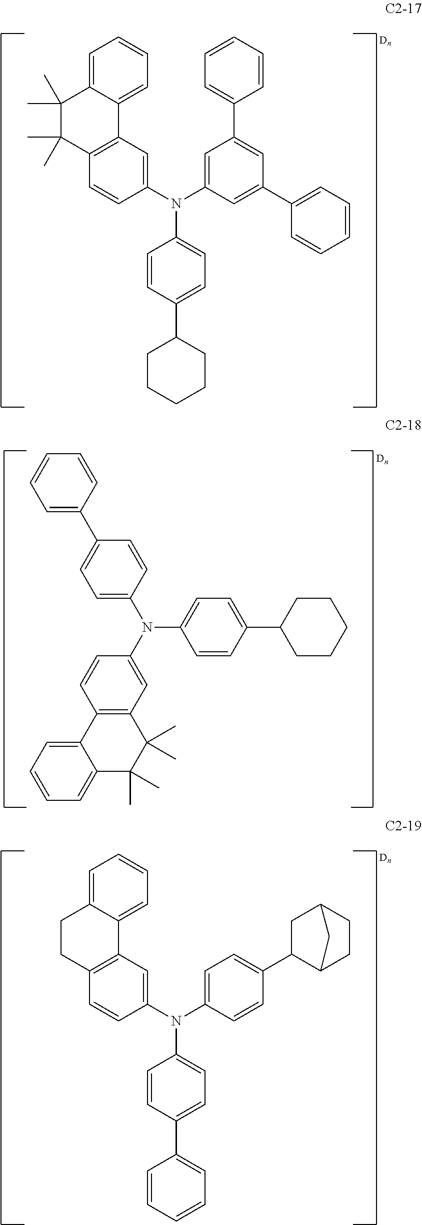

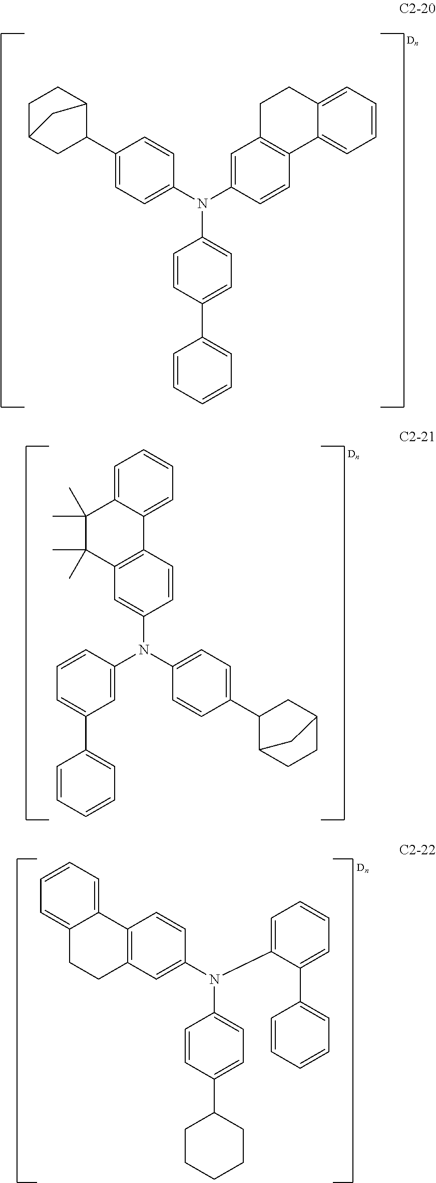

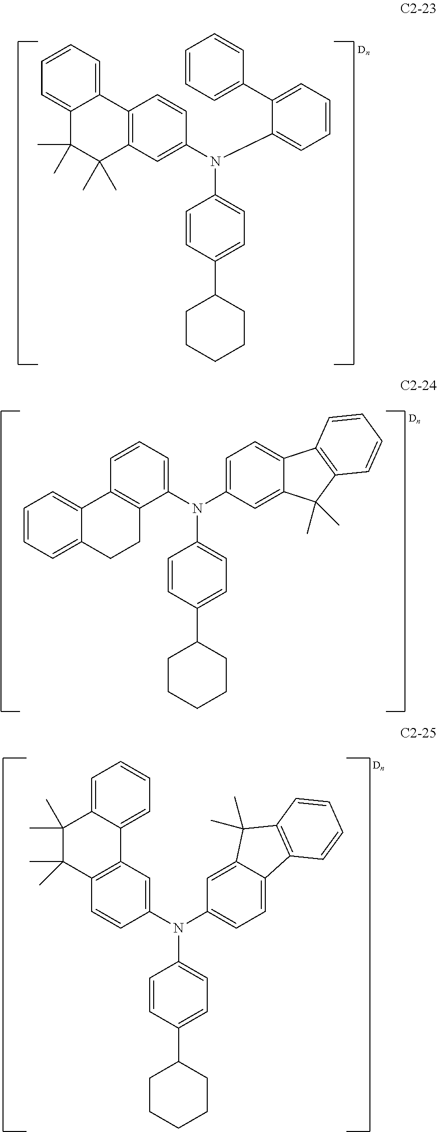

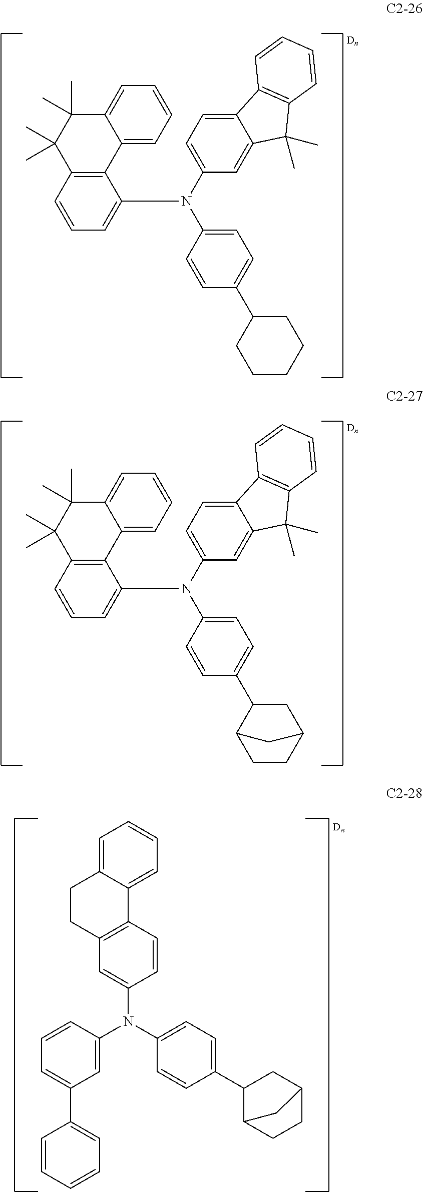

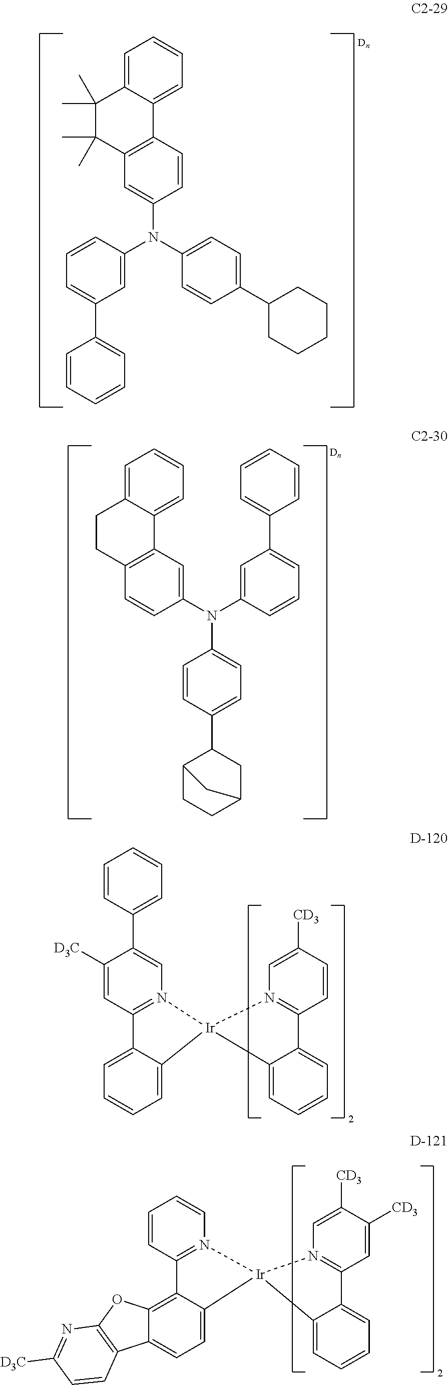

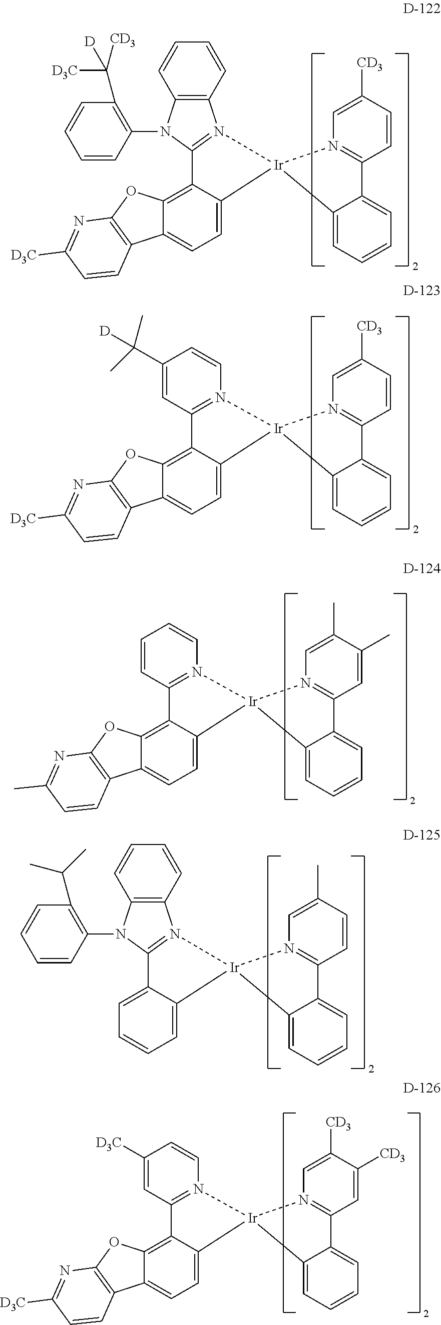

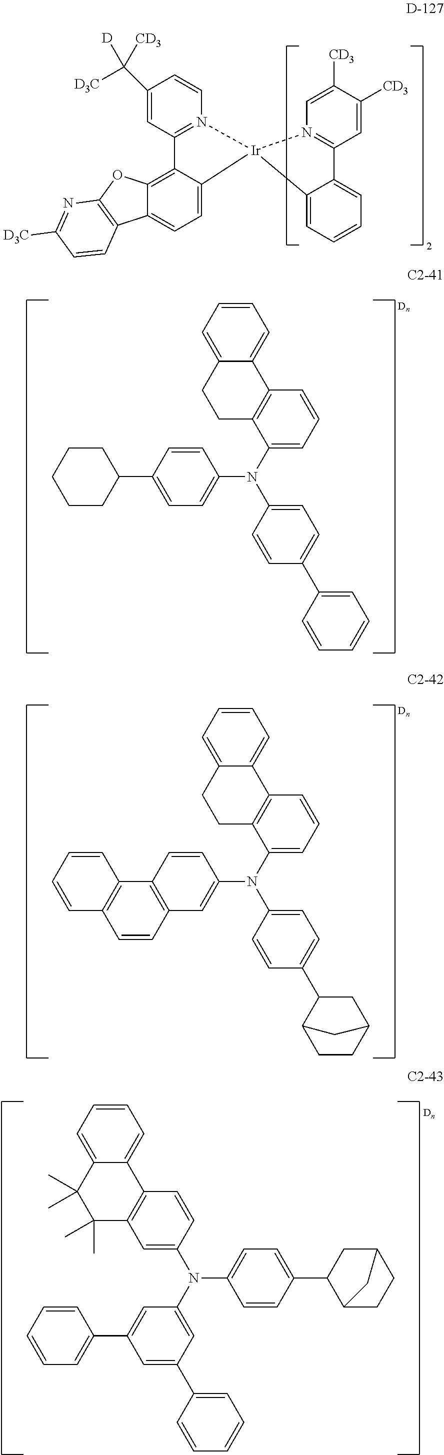

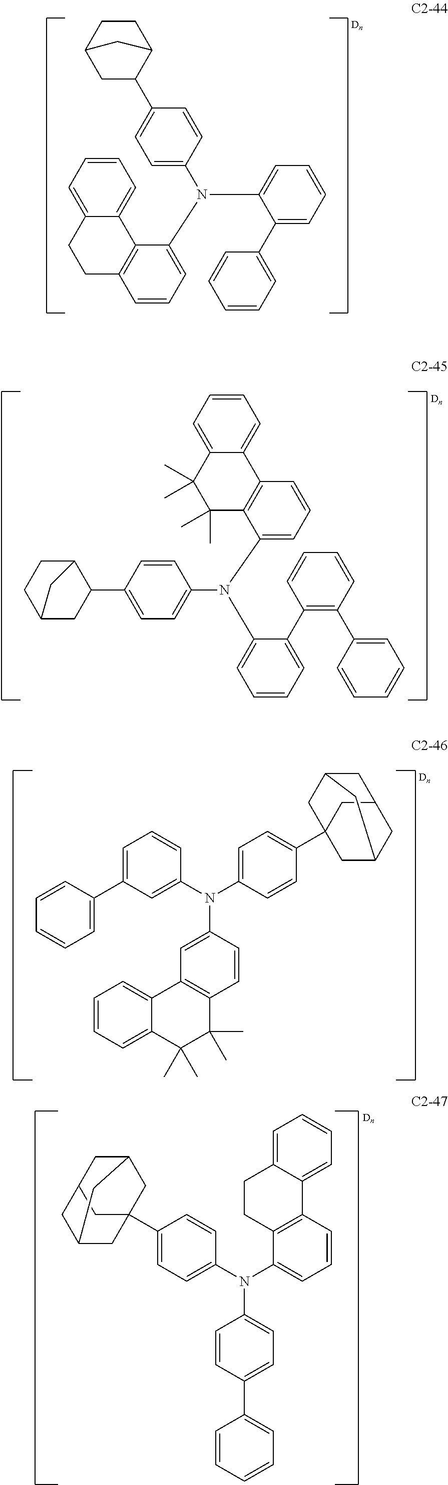

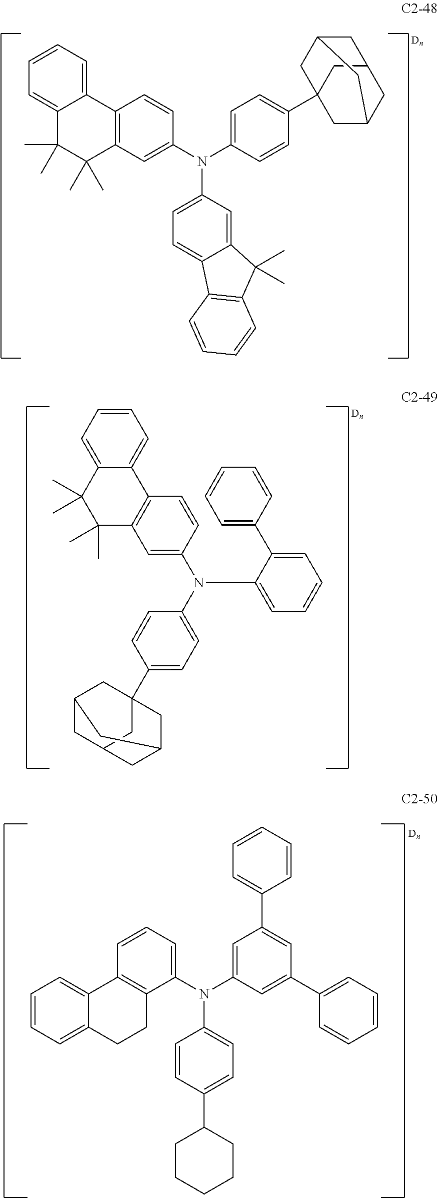

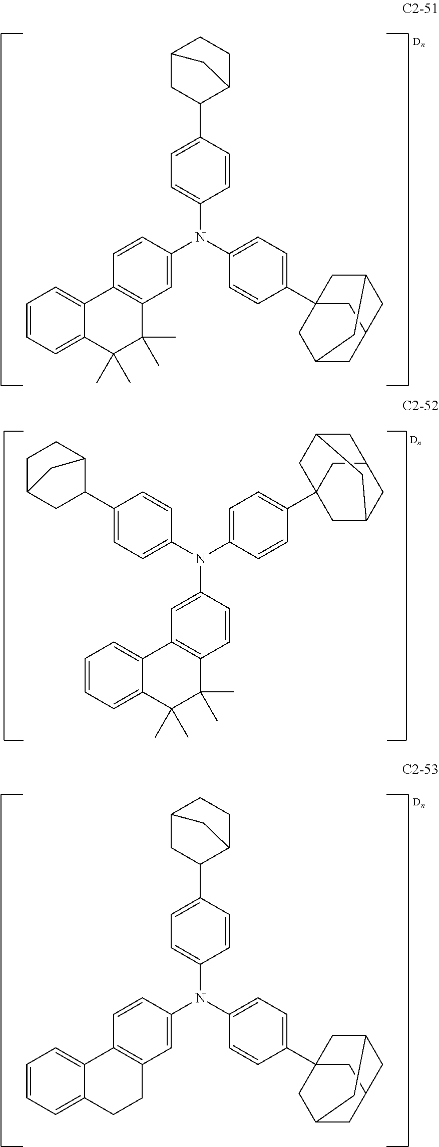

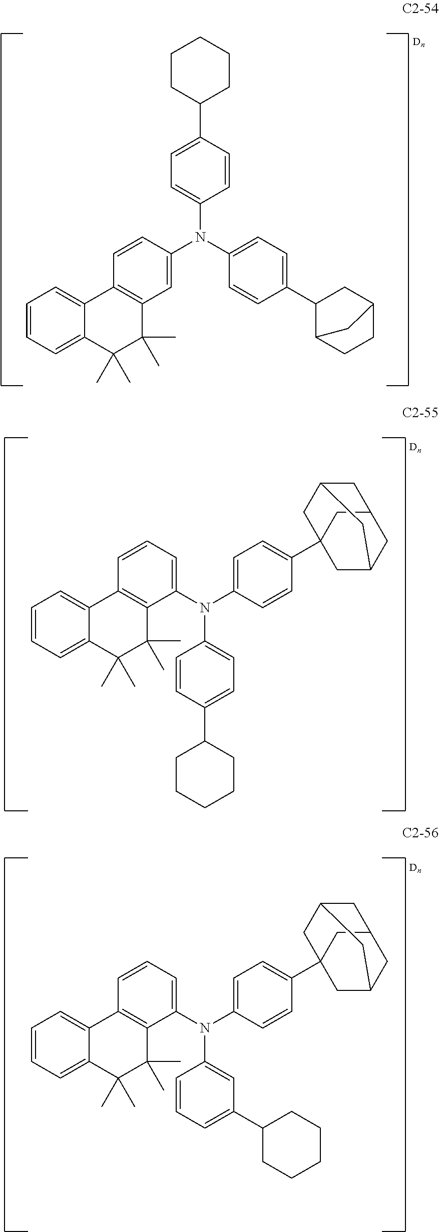

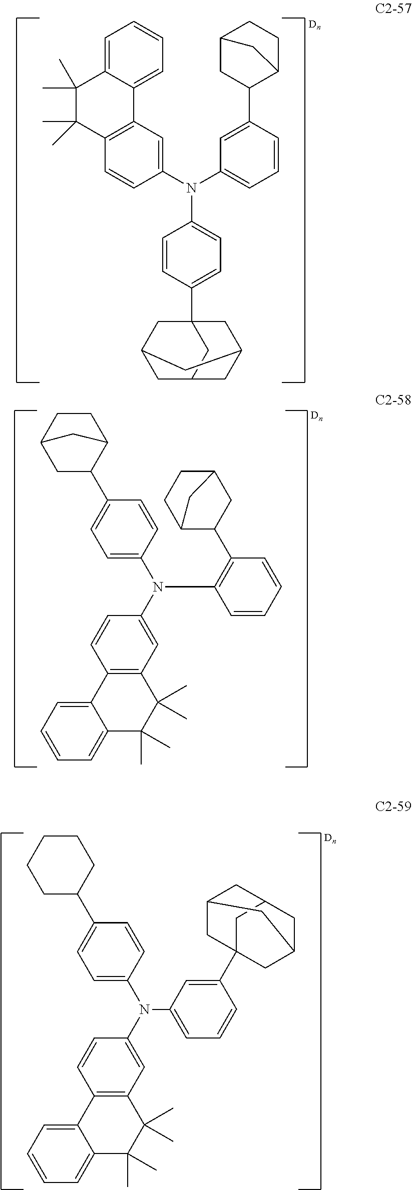

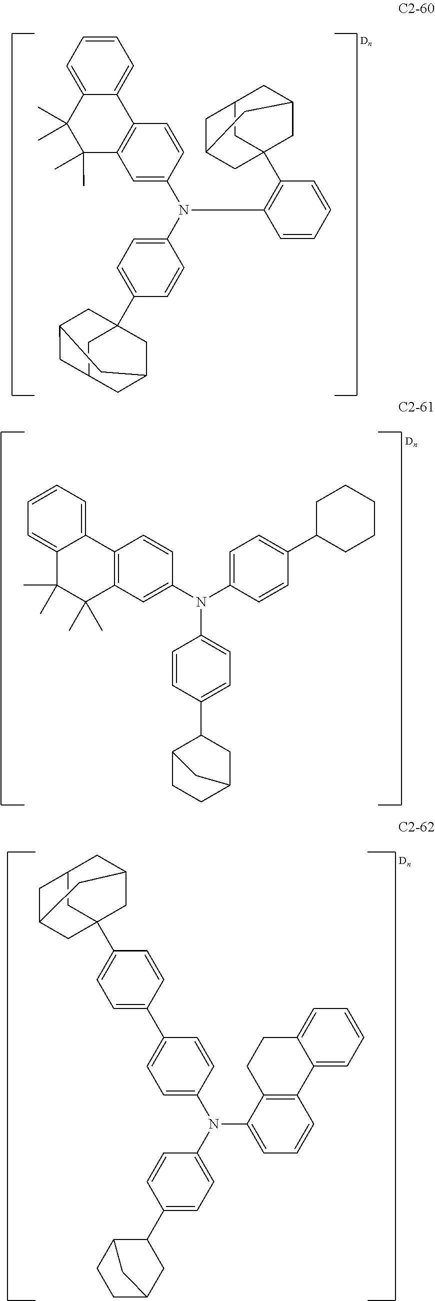

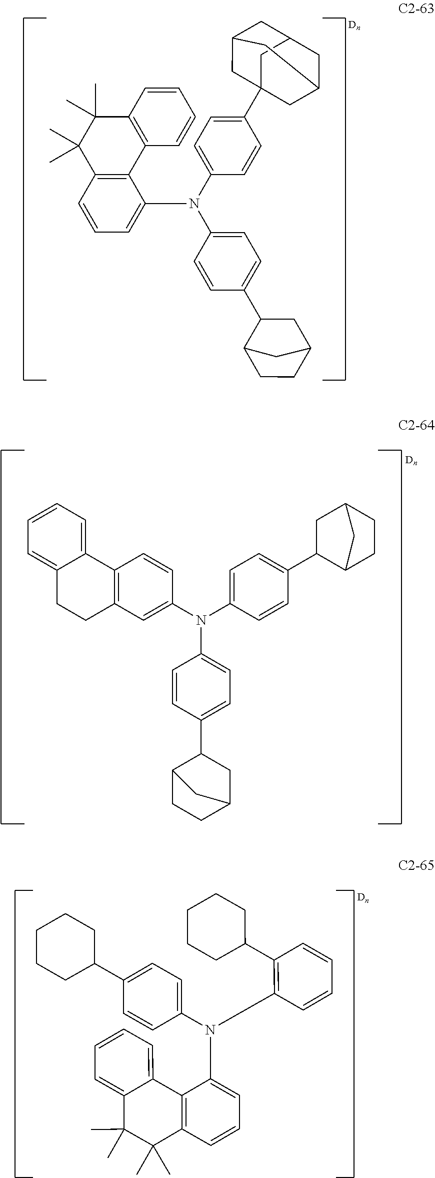

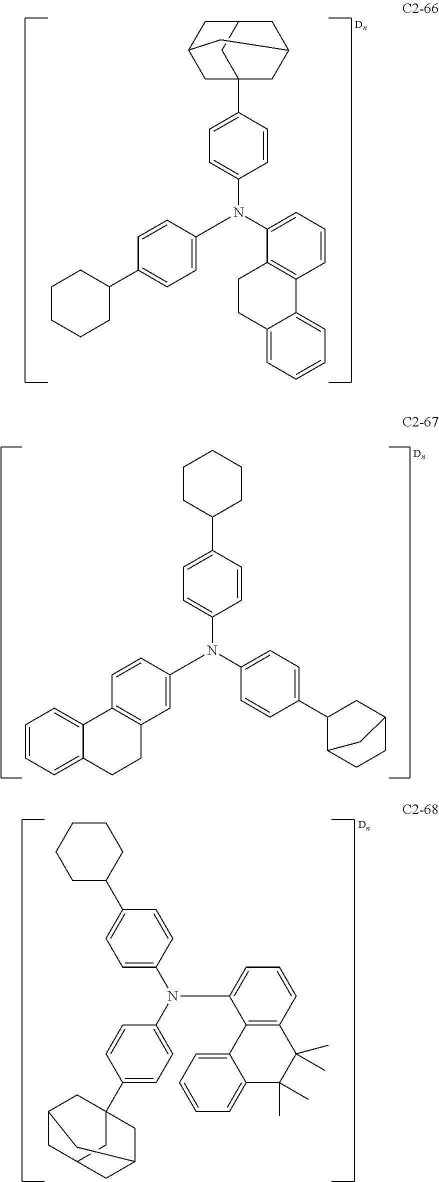

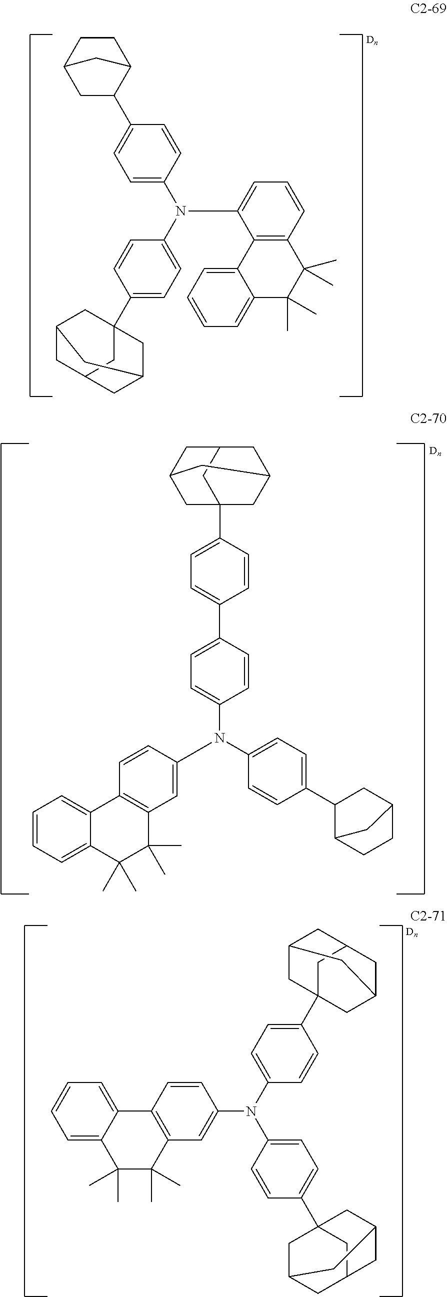

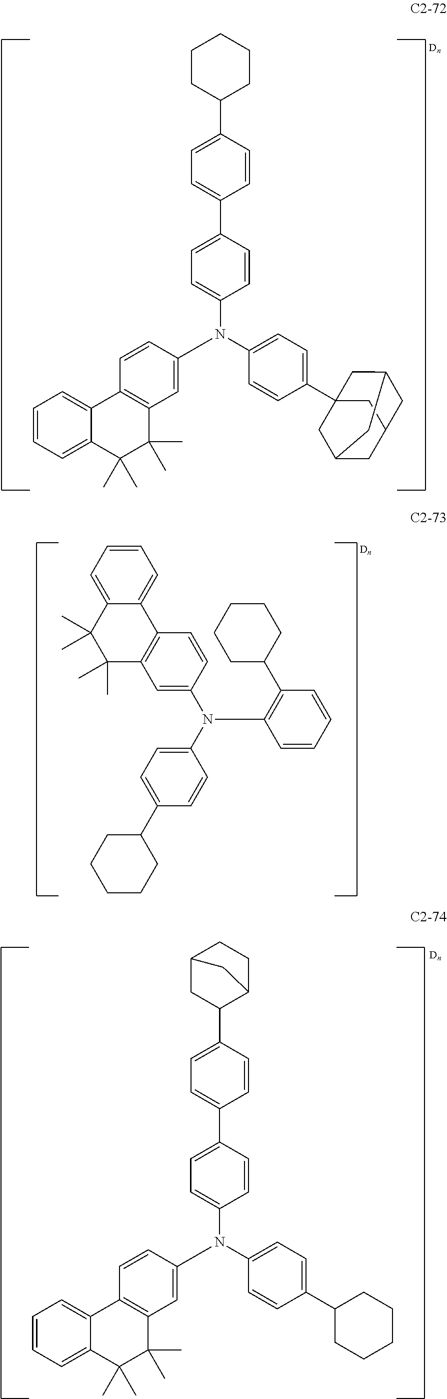

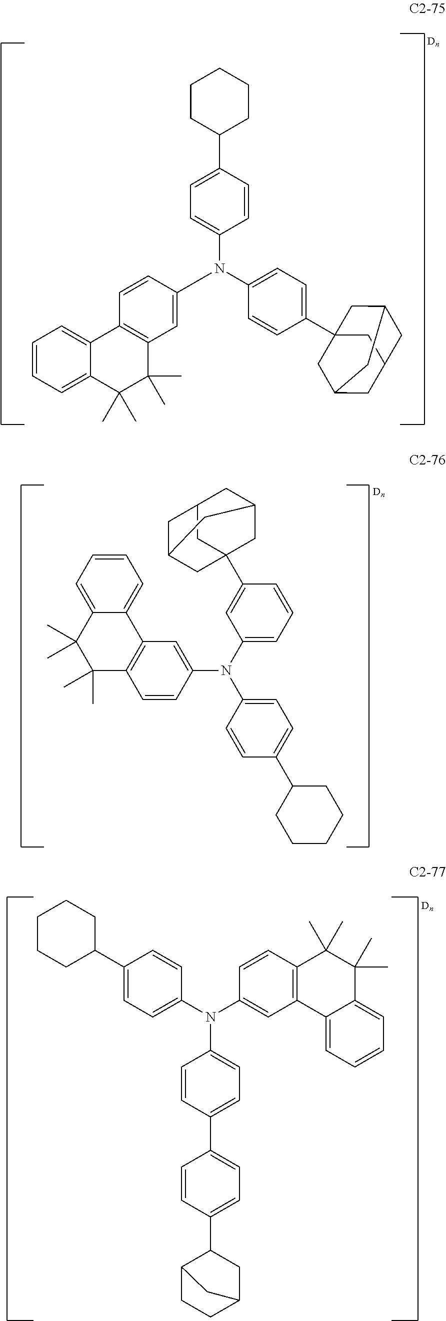

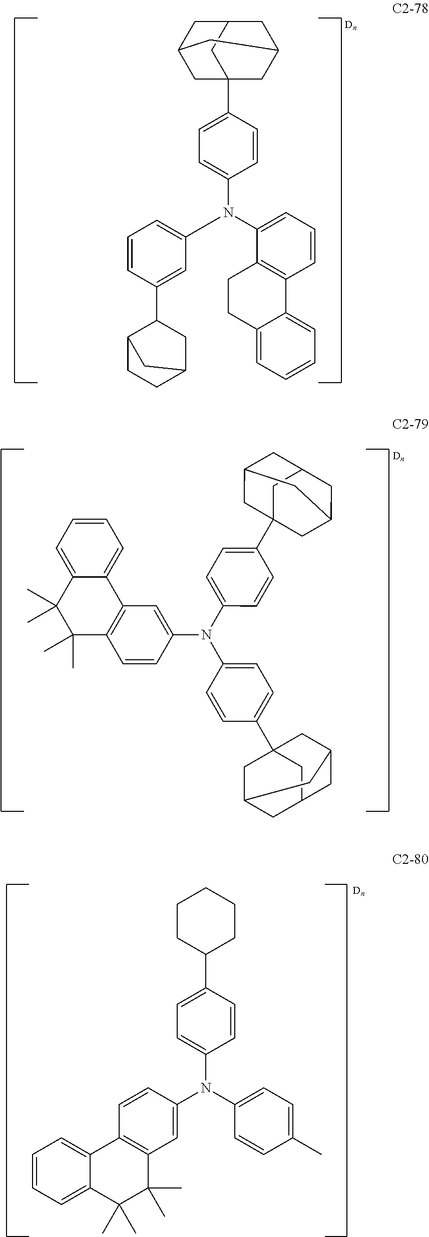

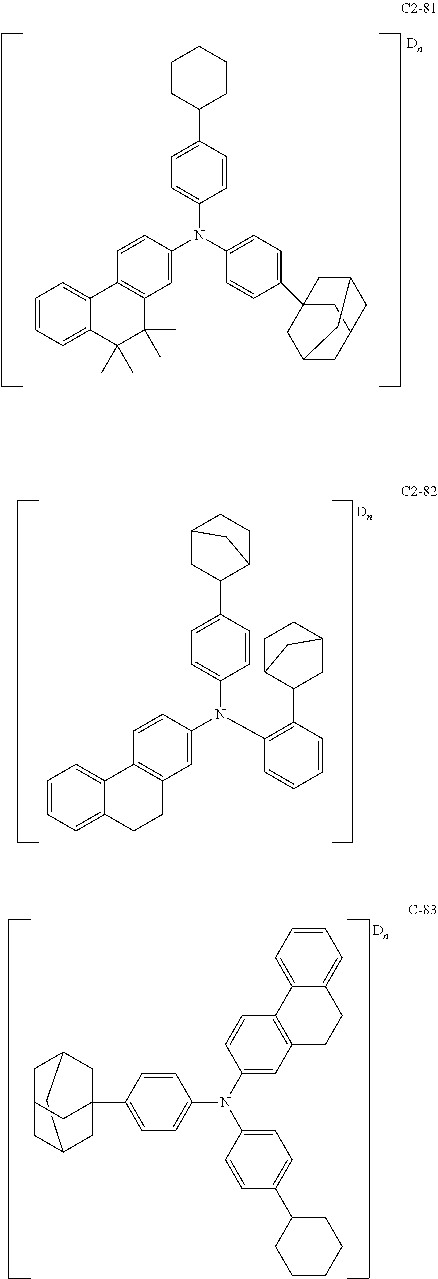

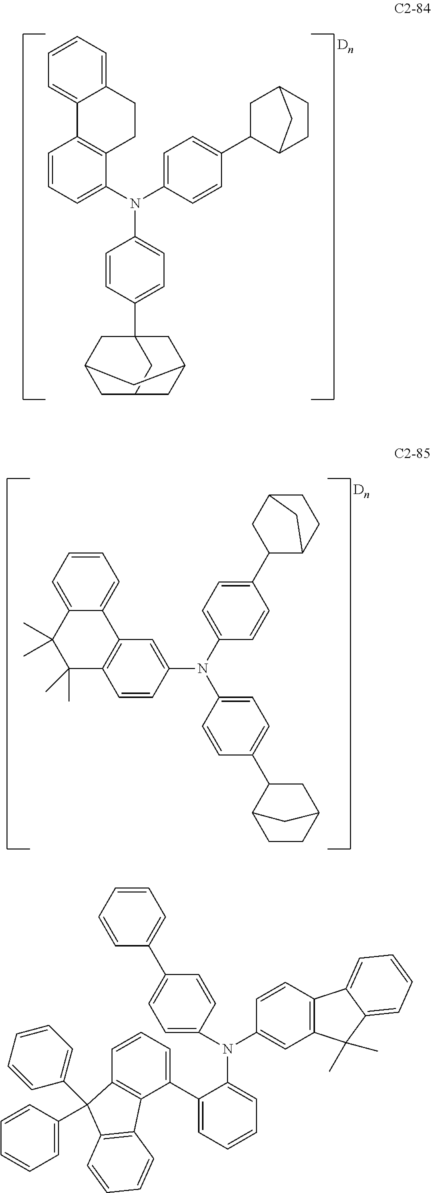

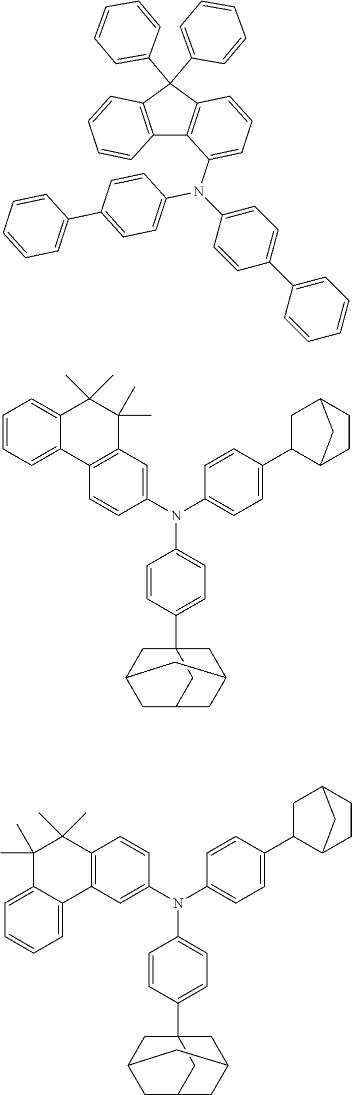 [SMILES: c1ccc(-c2ccc(N(c3ccc(-c4ccccc4)cc3)c3cccc4c3-c3ccccc3C4(c3ccccc3)c3ccccc3)cc2)cc1 ; CC1(C)c2ccccc2-c2ccc(N(c3ccc(C4CC5CCC4C5)cc3)c3ccc(C45CC6CC(CC(C6)C4)C5)cc3)cc2C1(C)C ; CC1(C)c2ccccc2-c2cc(N(c3ccc(C4CC5CCC4C5)cc3)c3ccc(C45CC6CC(CC(C6)C4)C5)cc3)ccc2C1(C)C]

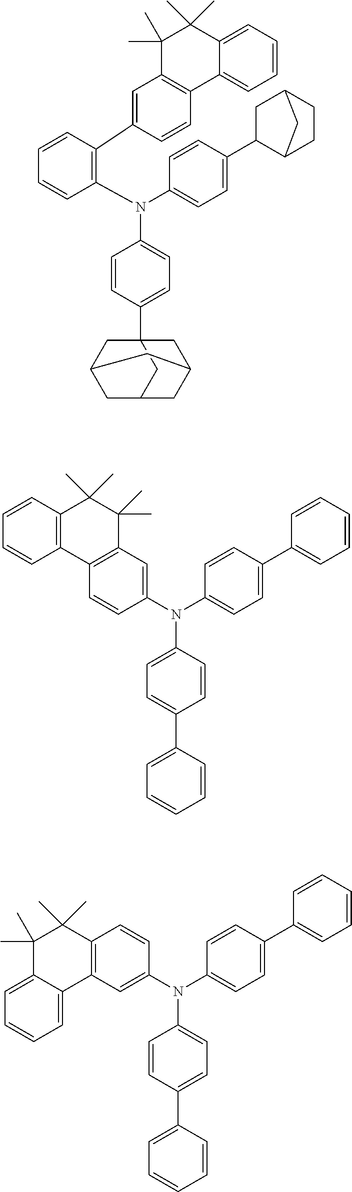 [SMILES: CC1(C)c2ccccc2-c2ccc(-c3ccccc3N(c3ccc(C4CC5CCC4C5)cc3)c3ccc(C45CC6CC(CC(C6)C4)C5)cc3)cc2C1(C)C ; CC1(C)c2ccccc2-c2ccc(N(c3ccc(-c4ccccc4)cc3)c3ccc(-c4ccccc4)cc3)cc2C1(C)C ; CC1(C)c2ccccc2-c2cc(N(c3ccc(-c4ccccc4)cc3)c3ccc(-c4ccccc4)cc3)ccc2C1(C)C]

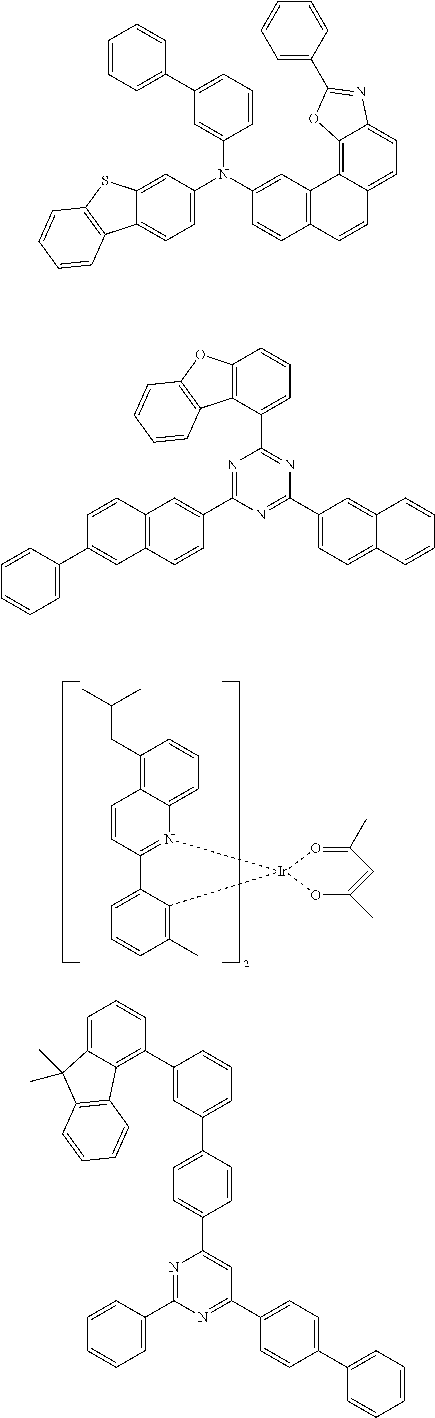

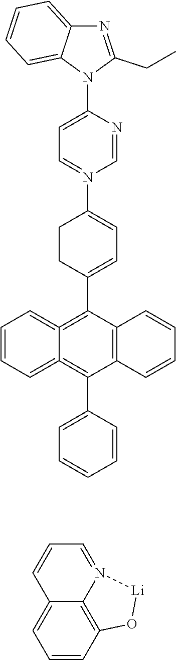

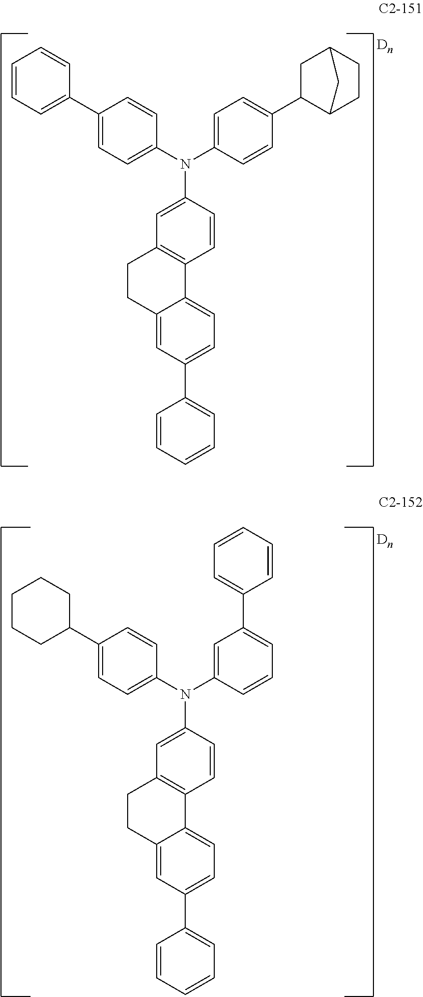

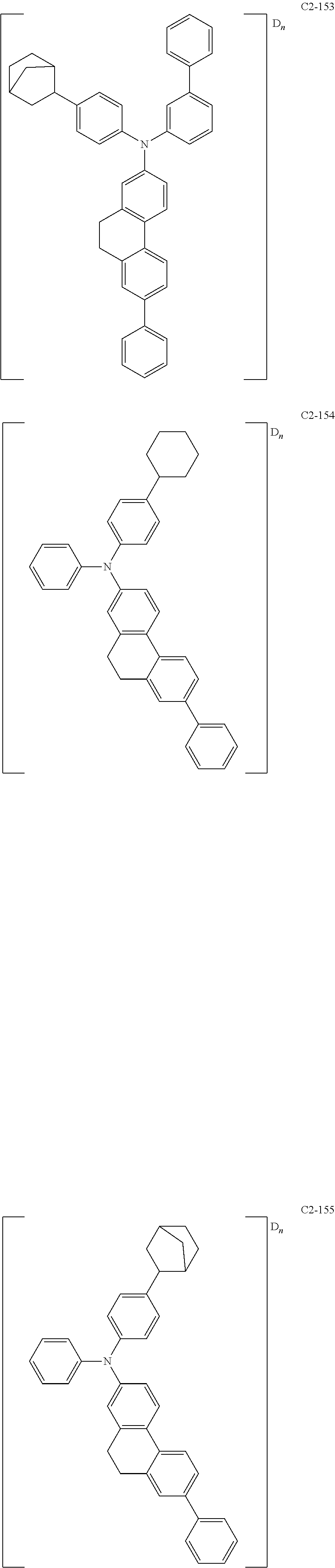

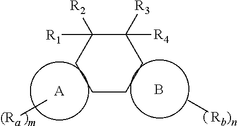

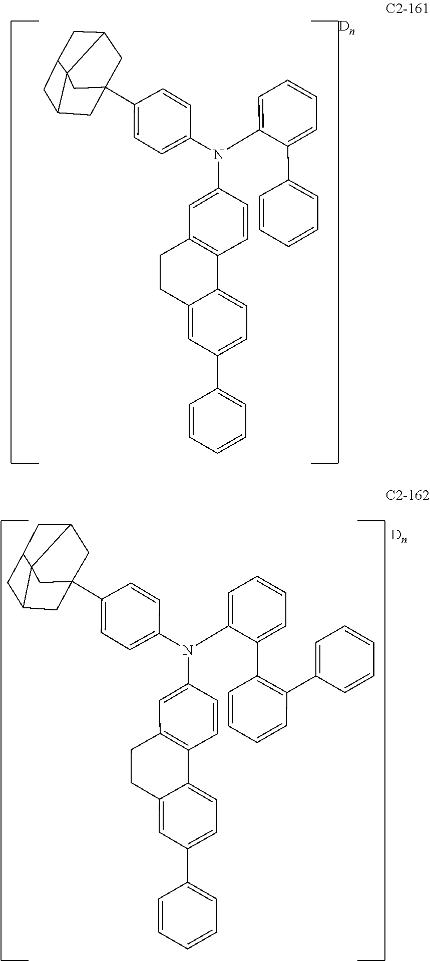

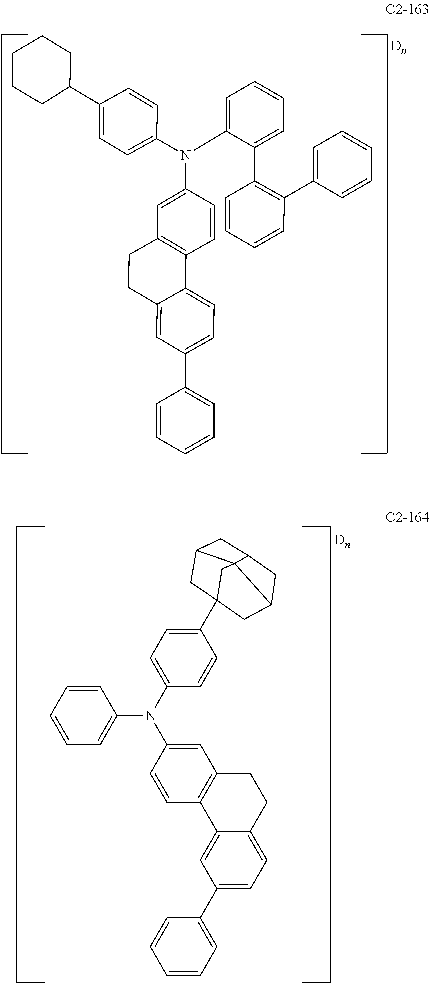

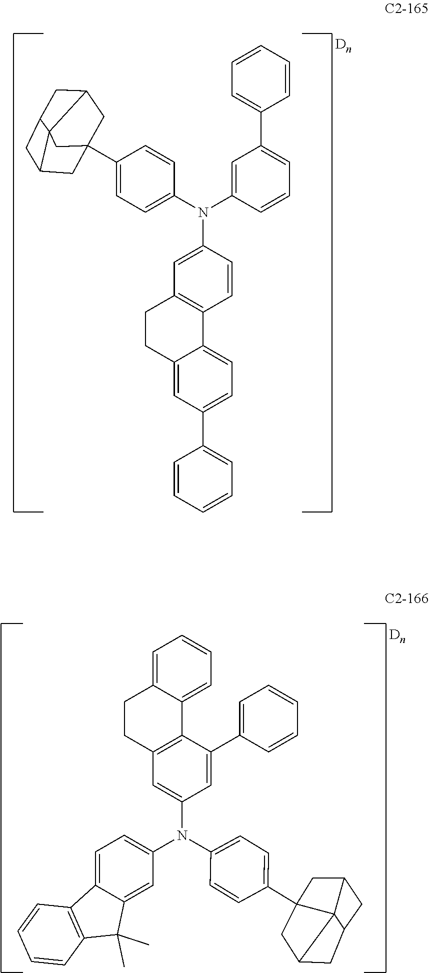

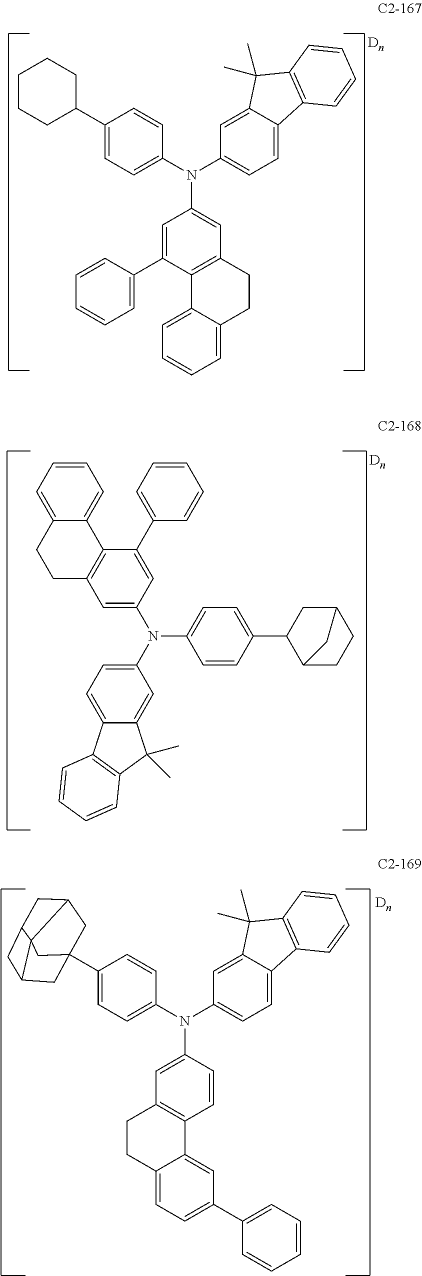

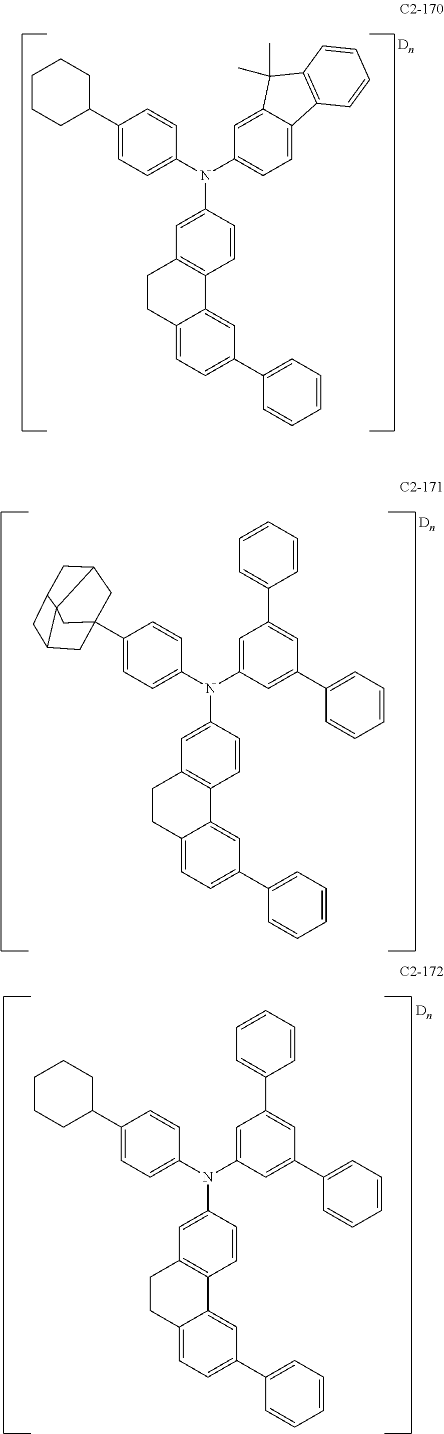

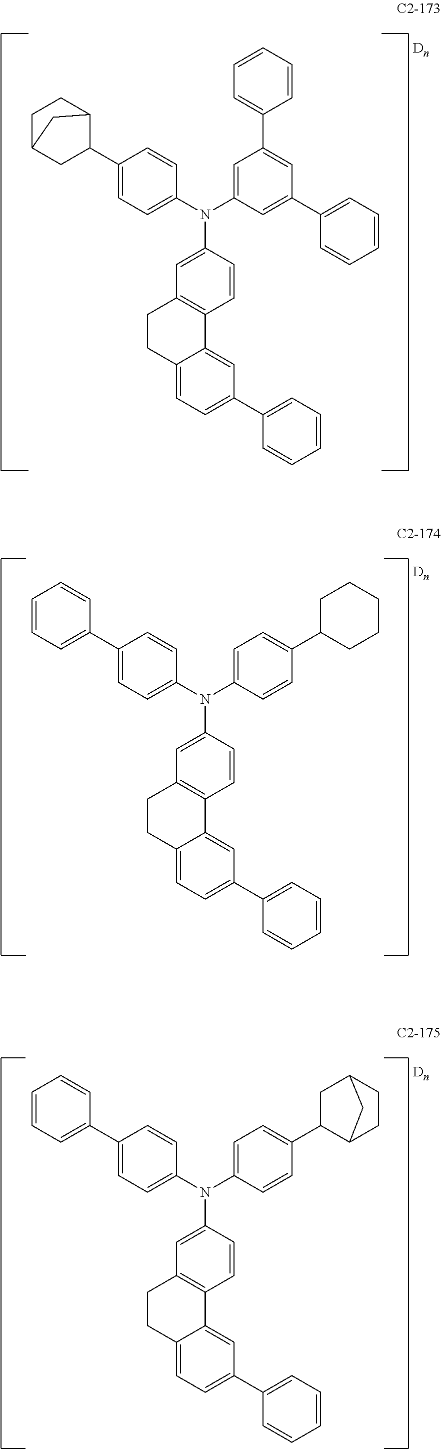

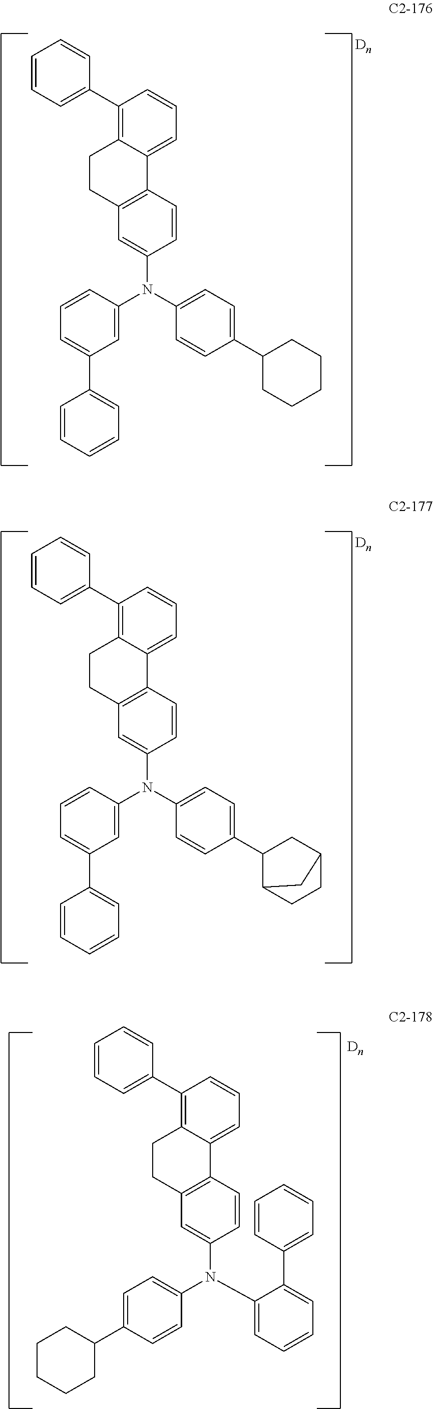

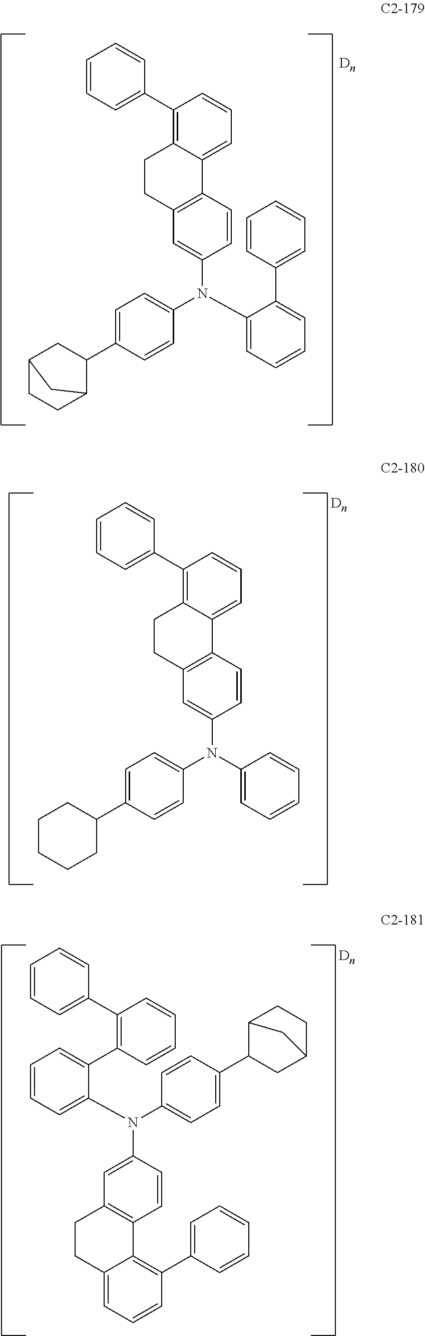

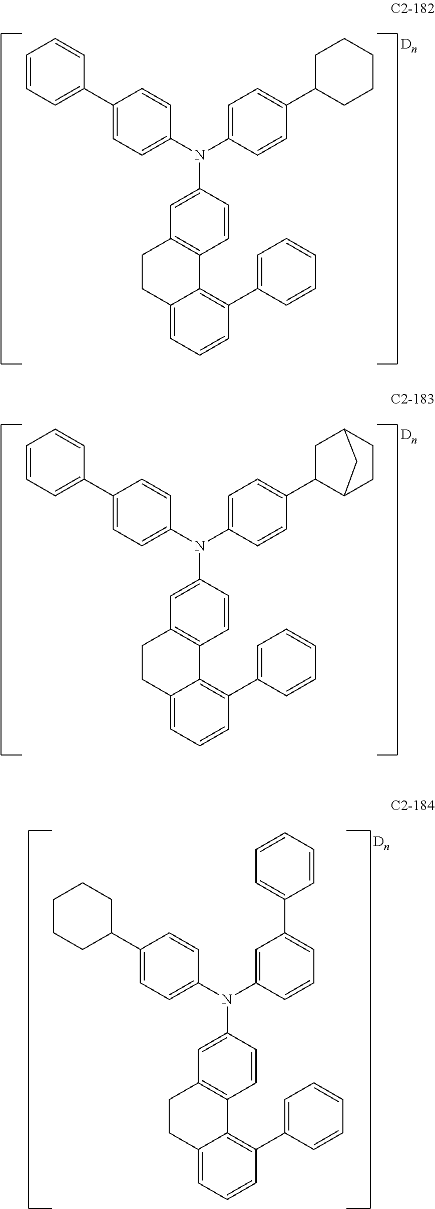

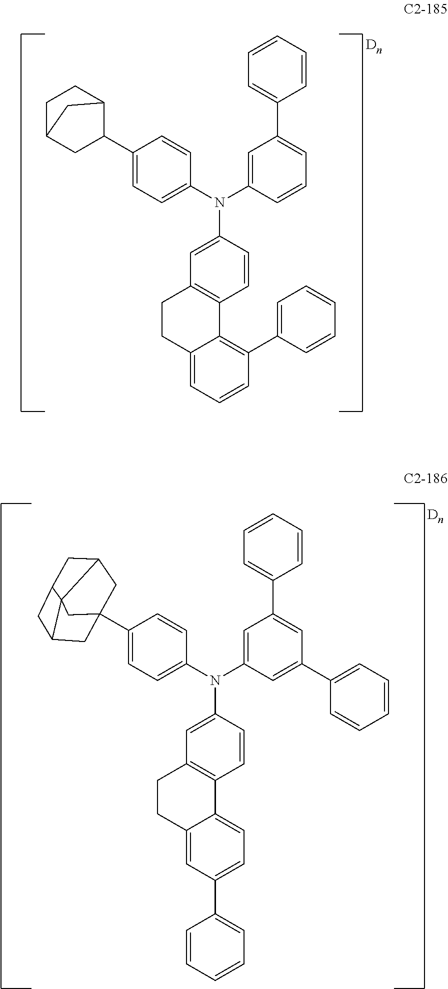

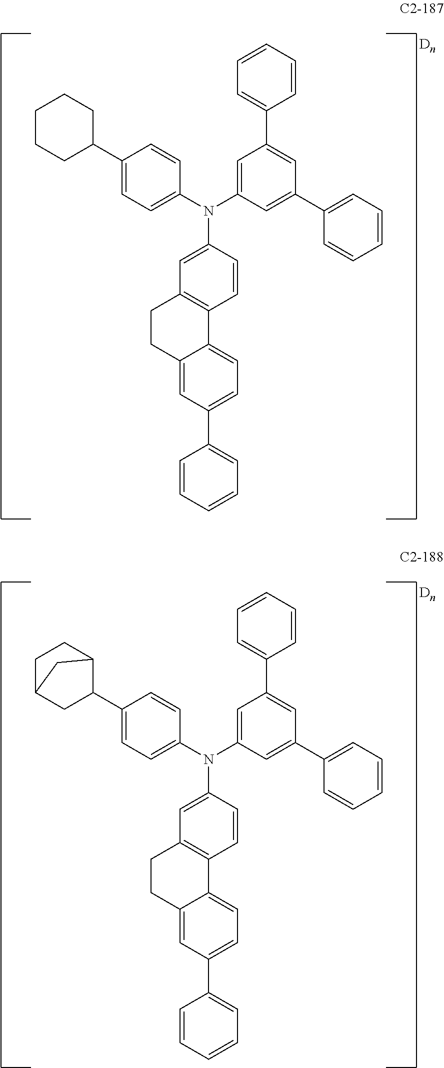

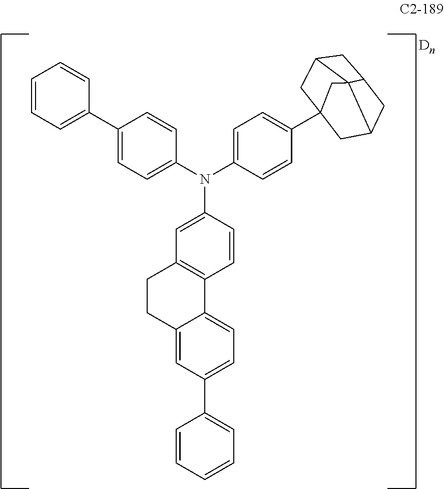

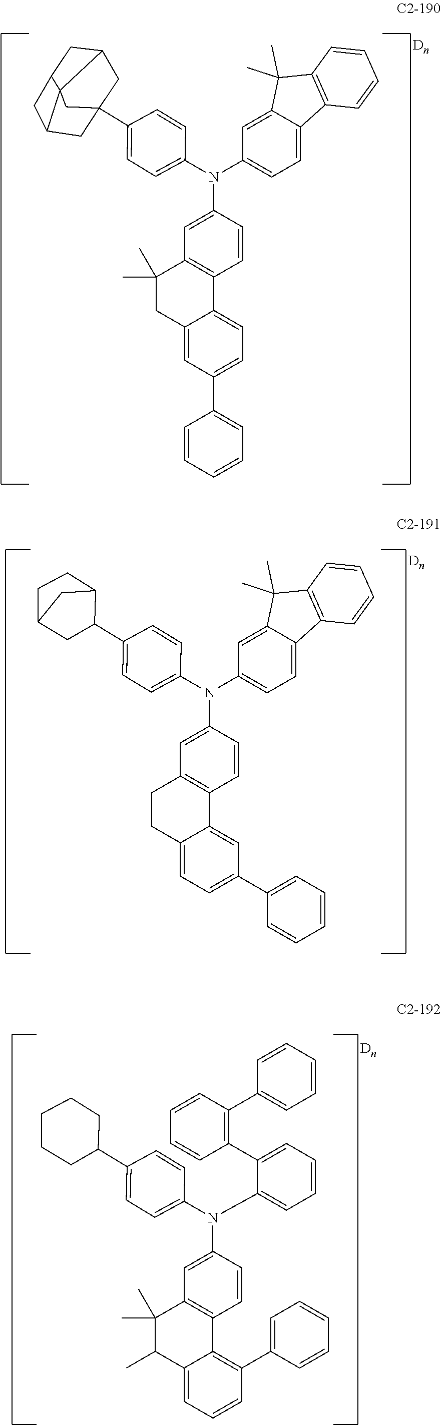

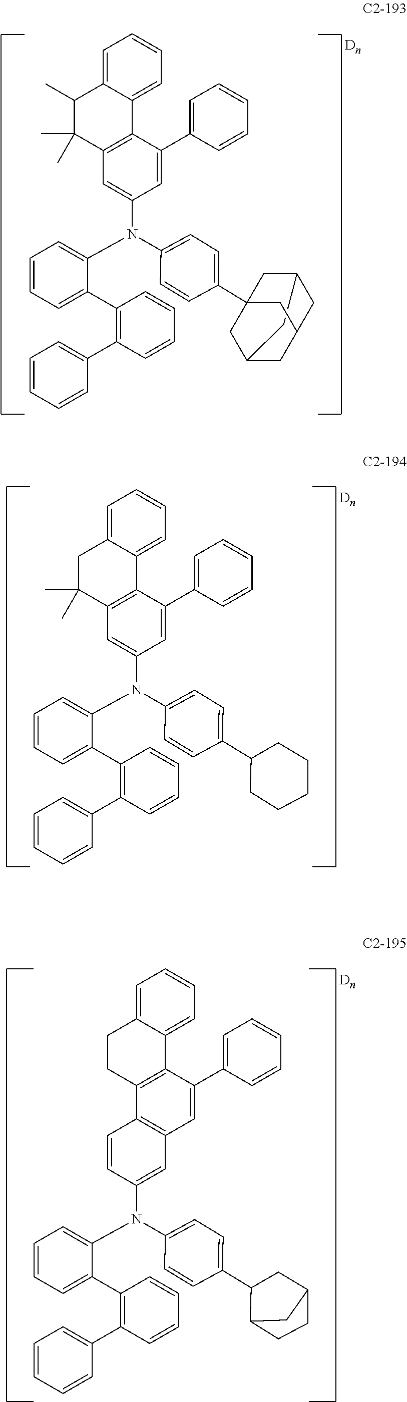

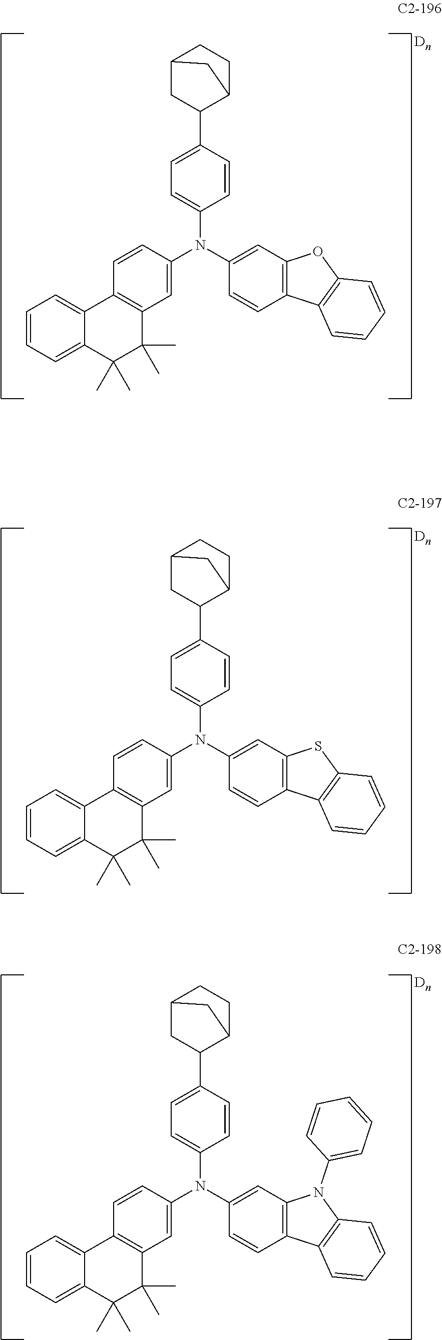

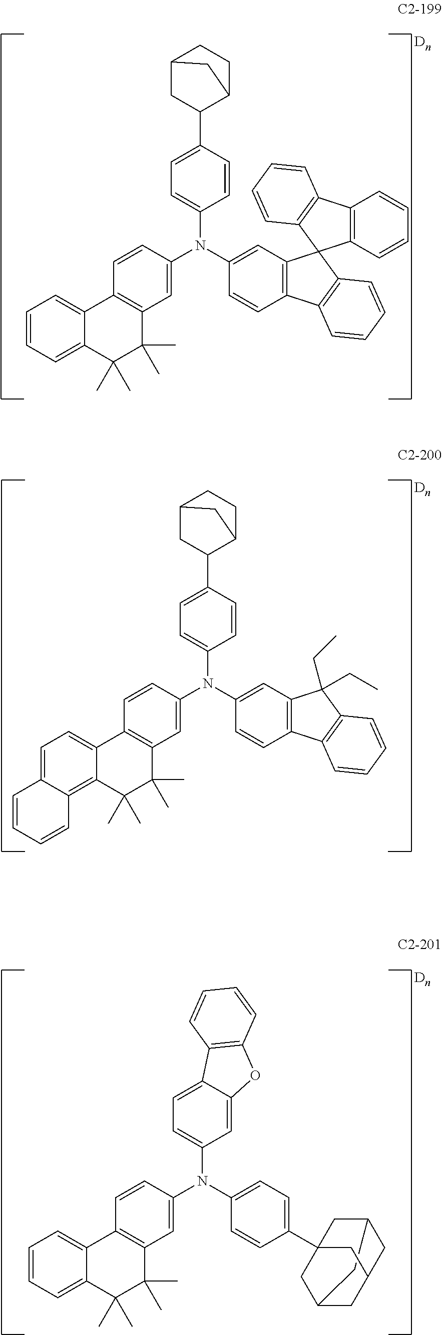

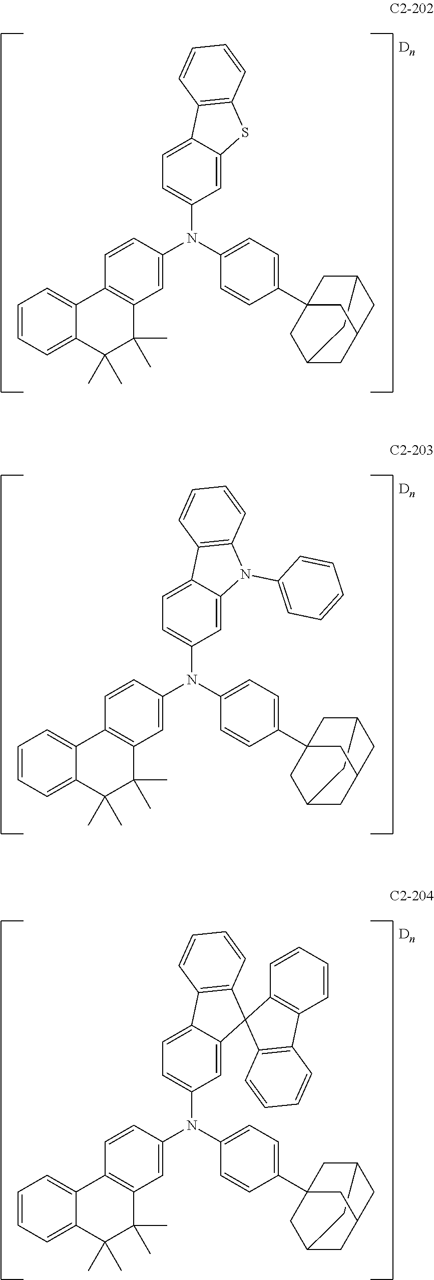

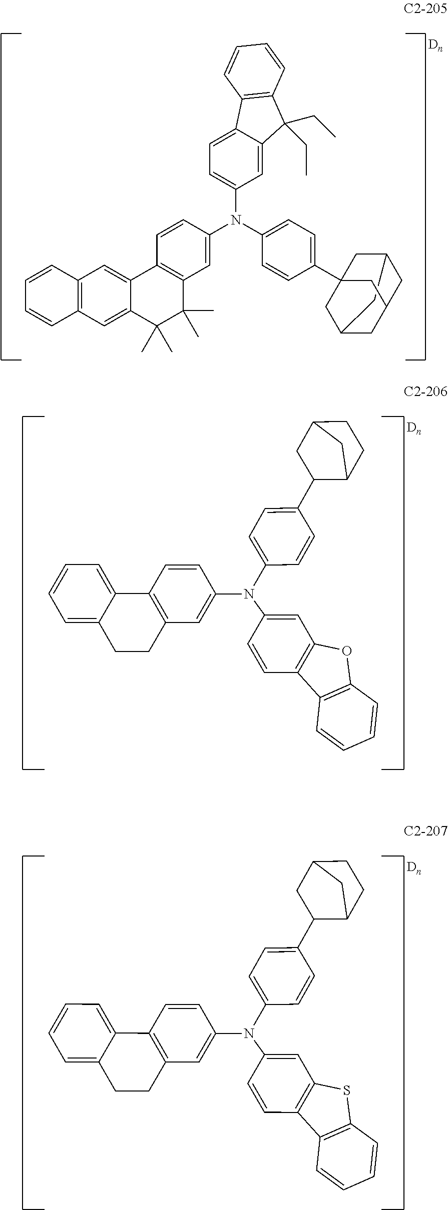

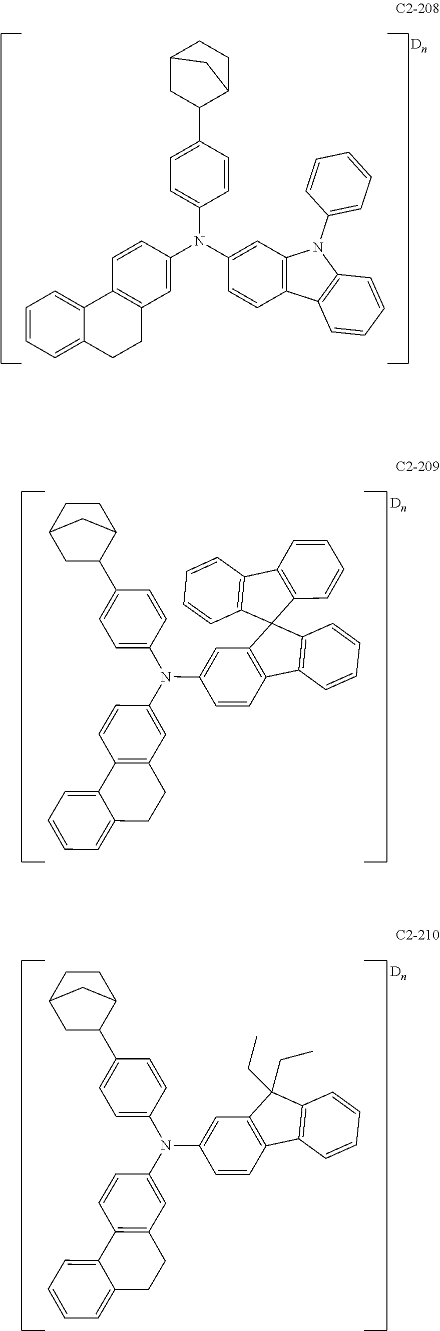

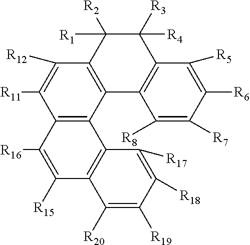

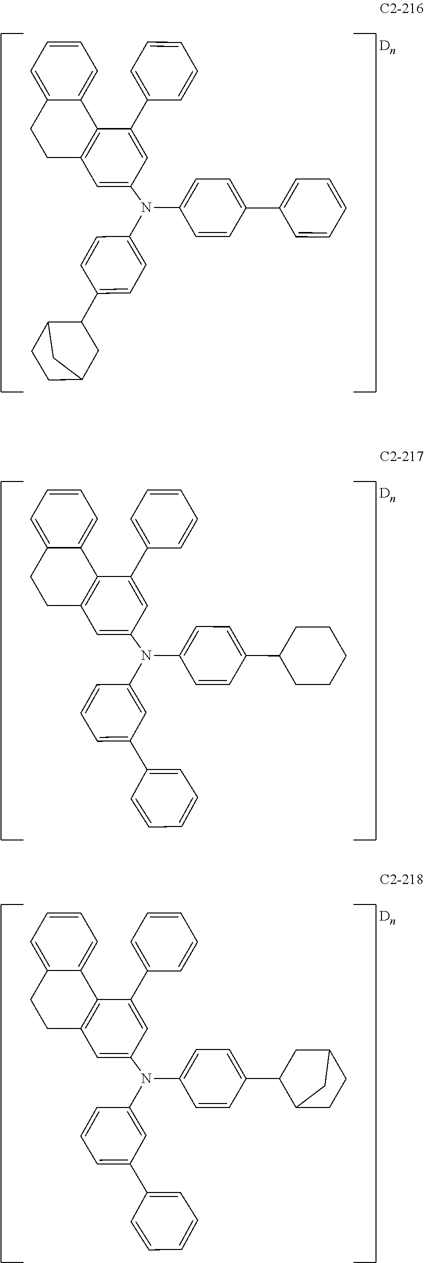

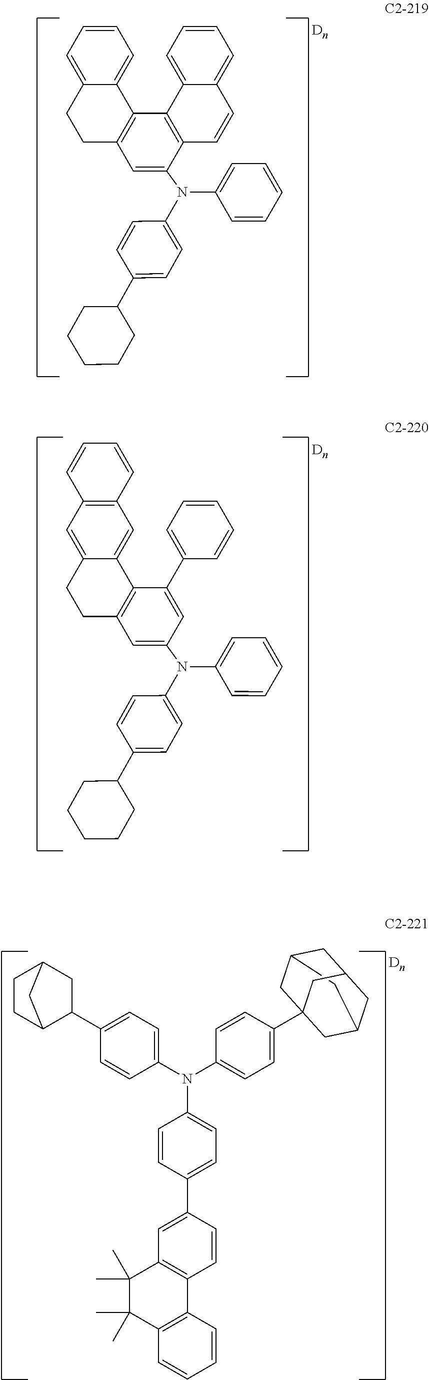

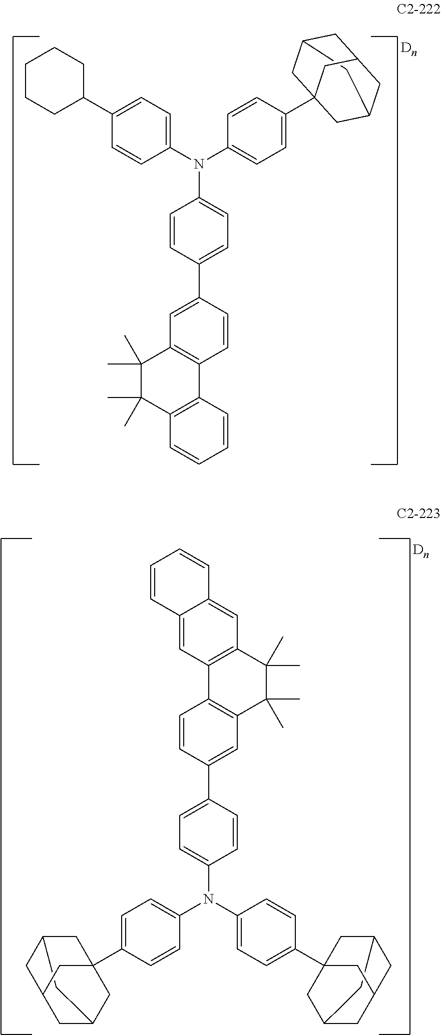

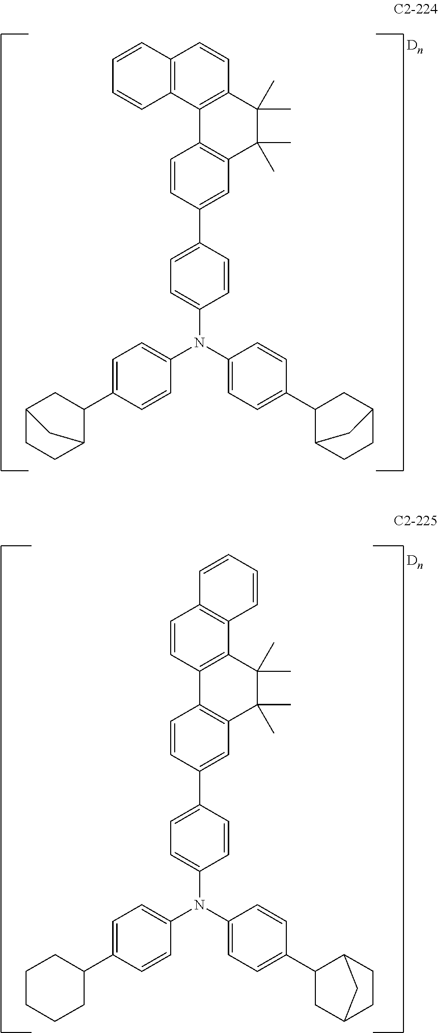

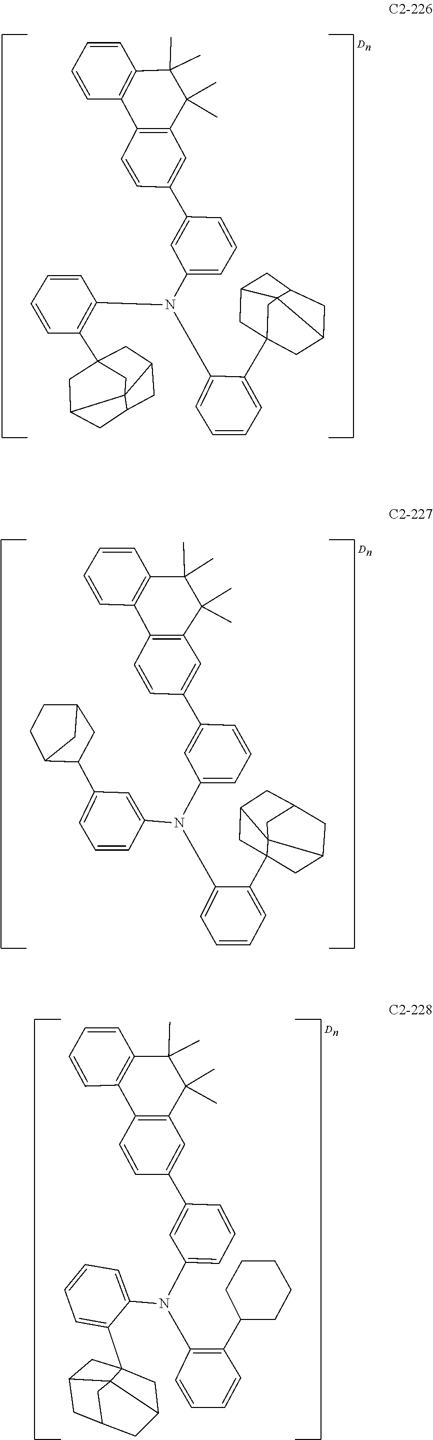

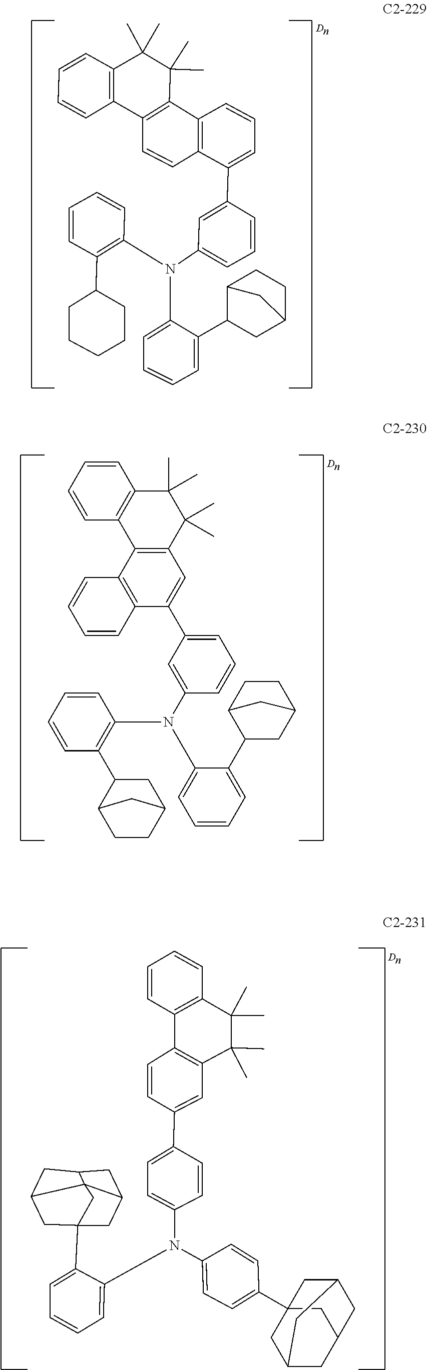

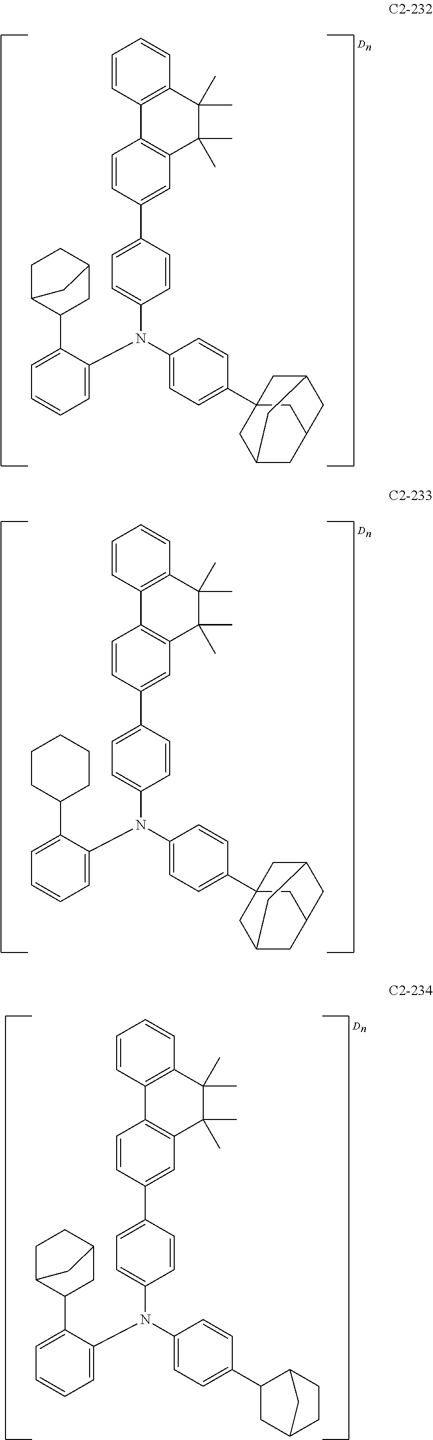

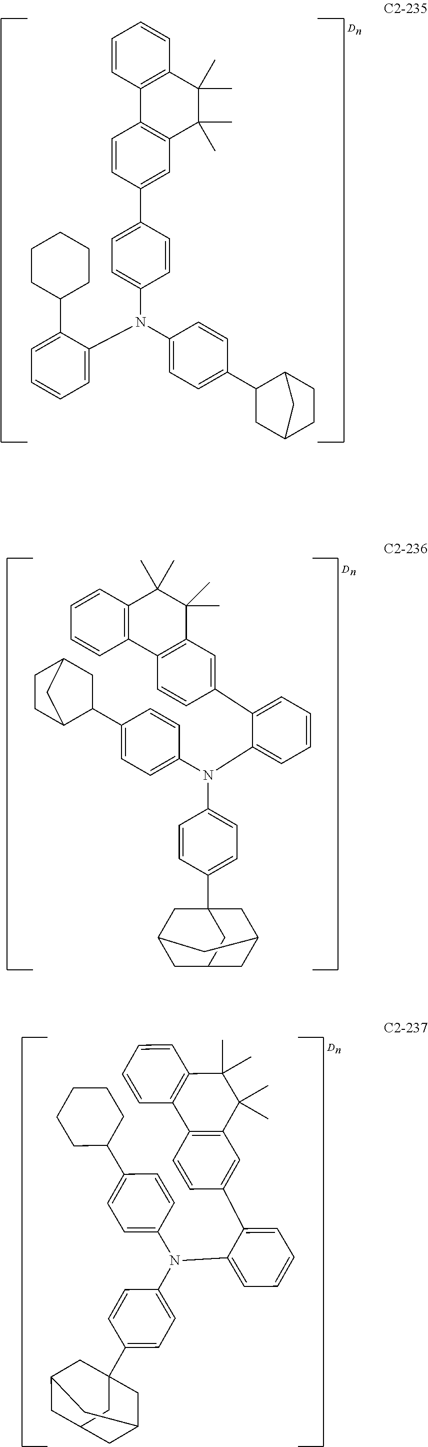

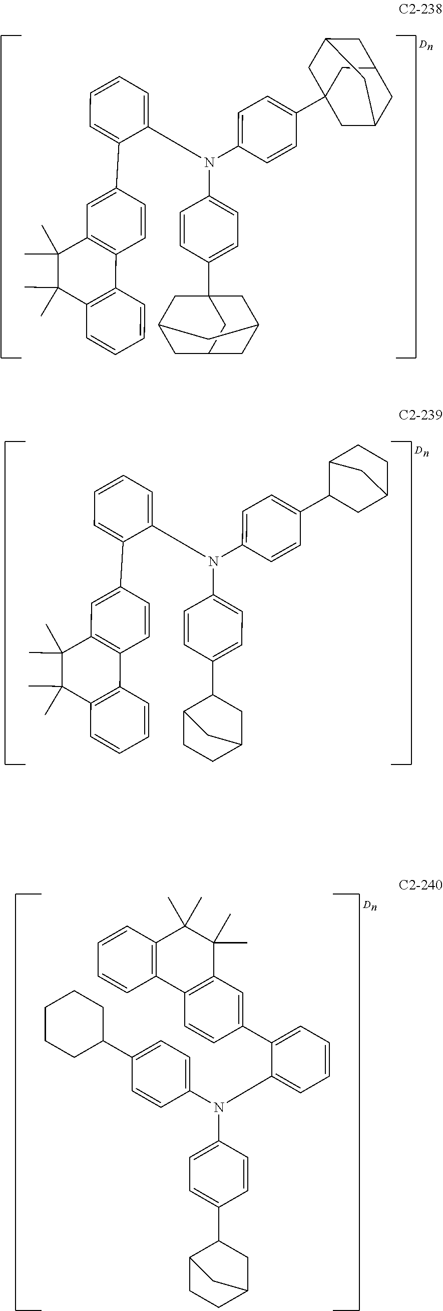

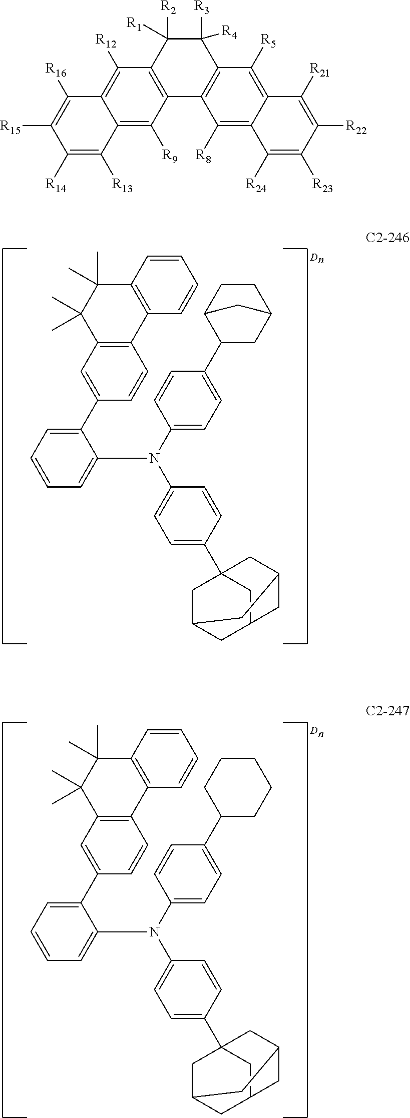

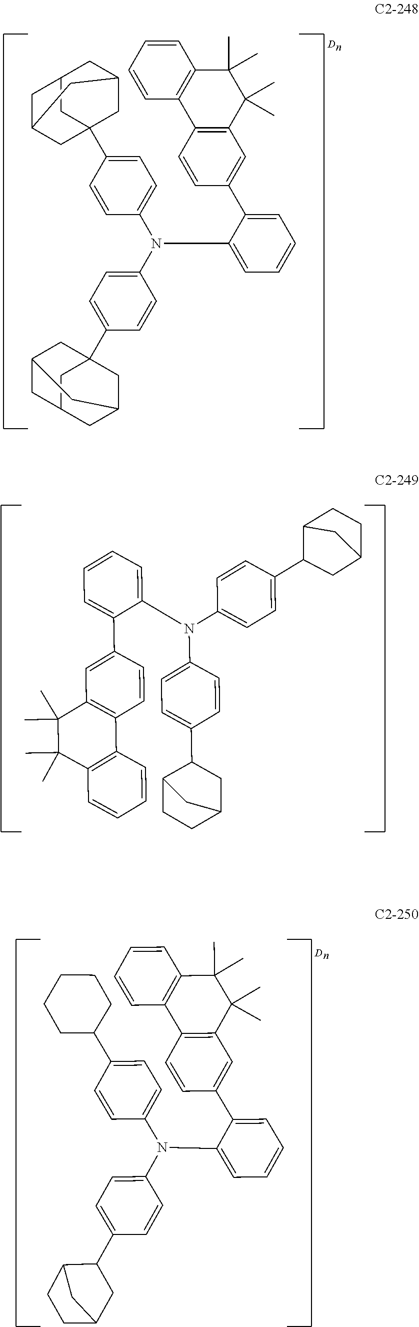

In the compounds above, Dₙ means that n hydrogen atoms are substituted with deuterium, wherein n is from 1 to the maximum number of hydrogen atoms in the compound.

The compound represented by Formula 1 according to the present disclosure may be produced by way of a synthetic method known to a person skilled in the art, for example, a synthetic method disclosed in Korean Patent Publication No. 10-2020-0092879 etc., but is not limited thereto.

Hereinafter, an organic electroluminescent device to which the aforementioned organic electroluminescent compound is applied will be described.

The organic electroluminescent device according to one embodiment includes a first electrode; a second electrode; and at least one organic layer(s) interposed between the first electrode and the second electrode. The organic layer includes a hole transport layer, a light-emitting layer, a hole auxiliary layer, an electron-blocking layer, and a light-emitting auxiliary layer. According to one embodiment, at least one of the hole transport layer, light-emitting layer, hole auxiliary layer, electron-blocking layer, and light-emitting auxiliary layer may include the organic electroluminescent compound represented by Formula 1. For example, the hole transport layer may include the organic electroluminescent compound of Formula 1. According to one embodiment, the organic electroluminescent materials of the present disclosure comprise at least one compound(s) of C-1 to C-750 and C2-1 to C2-250.

The organic electroluminescent materials may be included in the same organic layer, for example, the hole transport layer.

The light-emitting layer according to one embodiment may comprise a first host compound and a second host compound, wherein the weight ratio of the first host compound to the second host compound may be in the range of about 1:99 to about 99:1, preferably about 10:90 to about 90:10, more preferably about 30:70 to about 70:30, more preferably about 40:60 to about 60:40, and even more preferably about 50:50 in the light-emitting layer.

The organic layer may further comprise at least one layer selected from a hole injection layer, an electron transport layer, an electron injection layer, an interlayer, a hole-blocking layer, and an electron buffer layer, in addition to the hole transport layer, the light-emitting layer, the hole auxiliary layer, the electron-blocking layer, and the light-emitting auxiliary layer. The organic layer may further comprise an amine-based compound and/or an azine-based compound other than the light-emitting material according to the present disclosure. Specifically, the hole injection layer, the hole transport layer, the hole auxiliary layer, the light-emitting layer, the light-emitting auxiliary layer, or the electron-blocking layer may contain the amine-based compound, e.g., an arylamine-based compound and a styrylarylamine-based compound, etc., as a hole injection material, a hole transport material, a hole auxiliary material, a light-emitting material, a light-emitting auxiliary material, and an electron-blocking material. In addition, the electron transport layer, the electron injection layer, the electron buffer layer, and the hole-blocking layer may contain the azine-based compound as an electron transport material, an electron injection material, an electron buffer material, and a hole-blocking material. Also, the organic layer may further comprise at least one metal selected from the group consisting of metals of Group 1, metals of Group 2, transition metals of the 4^{th} period, transition metals of the 5^{th} period, lanthanides, and organic metals of the d-transition elements of the Periodic Table, or at least one complex compound comprising such a metal.

The organic electroluminescent compound according to one embodiment may be used as light-emitting materials for a white organic light-emitting device. The white organic light-emitting device allows various structures such as a parallel side-by-side arrangement method, a stacking arrangement method, or CCM (color conversion material) method, etc. according to the arrangement of R (red), G (green), YG (yellowish green), or B (blue) light-emitting units. In addition, the organic electroluminescent compound according to one embodiment may also be applied to the organic electroluminescent device comprising a QD (quantum dot).

One of the first electrode and the second electrode may be an anode, and the other may be a cathode. Herein, the first electrode and the second electrode may each be formed as a transmissive conductive material, a transflective conductive material, or a reflective conductive material. The organic electroluminescent device may be a top emission type, a bottom emission type, or a both-sides emission type according to the kinds of the material forming the first electrode and the second electrode.

A hole injection layer, a hole transport layer, an electron-blocking layer, or a combination thereof may be used between the anode and the light-emitting layer. The hole injection layer may be multi-layers in order to lower the hole injection barrier (or hole injection voltage) from the anode to the hole transport layer or the electron-blocking layer, wherein each of the multi-layers may use two compounds simultaneously. Also, the hole injection layer may be doped as a p-dopant. Also, the electron-blocking layer may be placed between the hole transport layer (or hole injection layer) and the light-emitting layer, and may confine the excitons within the light-emitting layer by blocking the overflow of electrons from the light-emitting layer to prevent light-emitting leakage. The hole transport layer or the electron-blocking layer may be multi-layers, wherein each layer may use a plurality of compounds.

An electron buffer layer, a hole-blocking layer, an electron transport layer, an electron injection layer, or a combination thereof may be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole-blocking layer may be placed between the electron transport layer (or electron injection layer) and the light-emitting layer, and blocks the arrival of holes to the cathode, thereby improving the probability of recombination of electrons and holes in the light-emitting layer. The hole-blocking layer or the electron transport layer may also be multi-layers, wherein each layer may use a plurality of compounds. Also, the electron injection layer may be doped with an n-dopant.

The light-emitting auxiliary layer may be placed between the anode and the light-emitting layer, or between the cathode and the light-emitting layer. When the light-emitting auxiliary layer is placed between the anode and the light-emitting layer, it may be used for promoting hole injection and/or hole transport, or for preventing overflow of electrons. When the light-emitting auxiliary layer is placed between the cathode and the light-emitting layer, it may be used for promoting electron injection and/or electron transport, or for preventing overflow of holes. In addition, the hole auxiliary layer may be placed between the hole transport layer (or hole injection layer) and the light-emitting layer, and may be effective to promote or block the hole transport rate (or the hole injection rate), thereby enabling the charge balance to be controlled. When an organic electroluminescent device includes two or more hole transport layers, the hole transport layer, which is further included, may be used as the hole auxiliary layer or the electron-blocking layer. The light-emitting auxiliary layer, the hole auxiliary layer, or the electron-blocking layer may have an effect of improving the efficiency and/or the lifespan of the organic electroluminescent device.

In the organic electroluminescent device of the present disclosure, preferably at least one layer (hereinafter, “a surface layer”) selected from a chalcogenide layer, a halogenated metal layer, and a metal oxide layer may be placed on an inner surface(s) of one or both of a pair of electrodes. Specifically, a chalcogenide (including oxides) layer of silicon and aluminum is preferably placed on an anode surface of an electroluminescent medium layer, and a halogenated metal layer or a metal oxide layer is preferably placed on a cathode surface of an electroluminescent medium layer. The operation stability for the organic electroluminescent device may be obtained by the surface layer. Preferably, the chalcogenide includes SiOₓ(1≤X≤2), AlOₓ(1≤X≤1.5), SiON, SiAlON, etc.; the halogenated metal includes LiF, MgF₂, CaF₂, a rare earth metal fluoride, etc.; and the metal oxide includes Cs₂O, Li₂O, MgO, SrO, BaO, CaO, etc.

In addition, in the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescent medium. Furthermore, the hole transport compound is oxidized to a cation, and thus it becomes easier to inject and transport holes from the mixed region to the electroluminescent medium. Preferably, the oxidative dopant may include various Lewis acids and acceptor compounds, and the reductive dopant may include alkali metals, alkali metal compounds, alkaline earth metals, rare earth metals, and mixtures thereof. Also, a reductive dopant layer may be employed as a charge generating layer to prepare an organic electroluminescent device having two or more light-emitting layers and emitting white light.

The organic electroluminescent device according to one embodiment of the present disclosure may be an organic electroluminescent device having a tandem structure. In the case of a tandem organic electroluminescent device according to one embodiment, a single light-emitting unit (light-emitting unit) may be formed in a structure in which two or more units are connected by a charge generation layer. The organic electroluminescent device may include a plurality of two or more light-emitting units, for example, a plurality of three or more light-emitting units, having first and second electrodes opposed to each other on a substrate and a light-emitting layer that is stacked between the first and second electrodes and emits light in a specific wavelength range. It may include a plurality of light-emitting units, and each of the light-emitting units may include a hole transport band, a light-emitting layer, and an electron transport band, and the hole transport band may include a hole injection layer and a hole transport layer, and the electron transport band may include an electron transport layer and an electron injection layer. According to one embodiment, three or more light-emitting layers may be included in the light-emitting unit. A plurality of light-emitting units may emit the same color or different colors. Additionally, one light-emitting unit may include one or more light-emitting layers, and the plurality of light-emitting layers may be light-emitting layers of the same or different colors. It may include one or more charge generation layers located between each light-emitting unit. The charge generation layer refers to the layer in which holes and electrons are generated when voltage is applied. When there are three or more light-emitting units, a charge generation layer may be located between each light-emitting unit. At this time, the plurality of charge generation layers may be the same as or different from one another. By disposing the charge generation layer between light-emitting units, current efficiency is increased in each light-emitting unit, and charges may be smoothly distributed. Specifically, the charge generation layer is provided between two adjacent stacks and can serve to drive a tandem organic electroluminescent device using only a pair of anodes and cathodes without a separate internal electrode located between the stacks.

The charge generation layer may be composed of an N-type charge generation layer and a P-type charge generation layer, and the N-type charge generation layer may be doped with an alkali metal, an alkaline earth metal, or a compound of an alkali metal and an alkaline earth metal. The alkali metal may include one selected from the group consisting of Li, Na, K, Rb, Cs, Fr, Yb, and combinations thereof, and the alkaline earth metal may include one selected from the group consisting of Be, Mg, Ca, Sr, Ba, Ra, and combinations thereof.

The P-type charge generation layer may be made of a metal or an organic material doped with a P-type dopant. For example, the metal may be made of one or two or more alloys selected from the group consisting of Al, Cu, Fe, Pb, Zn, Au, Pt, W, In, Mo, Ni, and Ti. Additionally, commonly used materials may be used as the P-type dopant and host materials used in the P-type doped organic material.

An organic electroluminescent device according to one embodiment may further comprise at least one dopant in the light-emitting layer.

The dopant comprised in the organic electroluminescent device of the present disclosure may be at least one phosphorescent or fluorescent dopant, preferably a phosphorescent dopant. The phosphorescent dopant material applied to the organic electroluminescent device of the present disclosure is not particularly limited, but may be preferably a complex compound(s) of a metal atom(s) selected from iridium (Ir), osmium (Os), copper (Cu), and platinum (Pt), more preferably an ortho-metallated complex compound(s) of a metal atom(s) selected from iridium (Ir), osmium (Os), copper (Cu), and platinum (Pt), and even more preferably ortho-metallated iridium complex compound(s).

The dopant comprised in the organic electroluminescent device of the present disclosure may use the compound represented by the following Formula 101, but is not limited thereto.

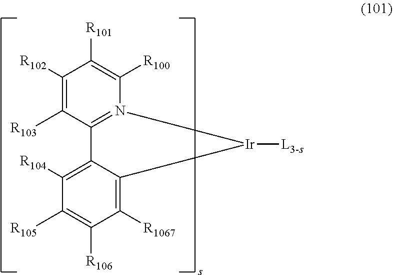

In Formula 101,
L is any one selected from the following Structures 1 to 3:

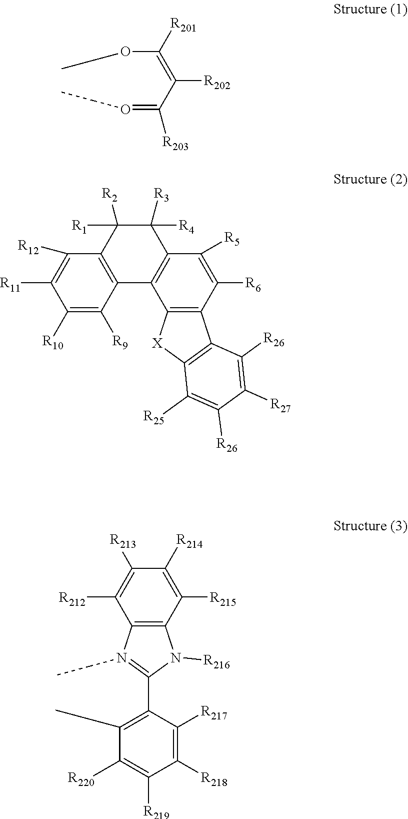

R₁₀₀ to R₁₀₃ each independently represent hydrogen, deuterium, halogen, (C₁-C₃₀)alkyl substituted or unsubstituted with deuterium and/or halogen, a substituted or unsubstituted (C₃-C₃₀)cycloalkyl, a substituted or unsubstituted (C₆-C₃₀)aryl, cyano, a substituted or unsubstituted (3- to 30-membered)heteroaryl, or a substituted or unsubstituted (C₁-C₃₀)alkoxy; or may be linked to the adjacent substituents to form a ring(s), for example, to form a ring(s) with a pyridine, e.g., a substituted or unsubstituted quinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline; R₁₀₄ to R₁₀₇ each independently represent hydrogen, deuterium, halogen, (C₁-C₃₀)alkyl substituted or unsubstituted with deuterium and/or halogen, a substituted or unsubstituted (C₃-C₃₀)cycloalkyl, a substituted or unsubstituted (C₆-C₃₀)aryl, a substituted or unsubstituted (3- to 30-membered)heteroaryl, cyano, or a substituted or unsubstituted (C₁-C₃₀)alkoxy; or may be linked to an adjacent substituent(s) to form a substituted or unsubstituted ring(s), for example, to form a ring(s) with a benzene, e.g., a substituted or unsubstituted naphthalene, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine;

R₂₀₁ to R₂₂₀ each independently represent hydrogen, deuterium, halogen, (C₁-C₃₀)alkyl substituted or unsubstituted with deuterium and/or halogen, a substituted or unsubstituted (C₃-C₃₀)cycloalkyl, or a substituted or unsubstituted (C₆-C₃₀)aryl; or may be linked to an adjacent substituent(s) to form a substituted or unsubstituted ring(s); and
represents an integer of 1 to 3.

Specifically, the specific examples of the dopant compound include the following, but are not limited thereto.

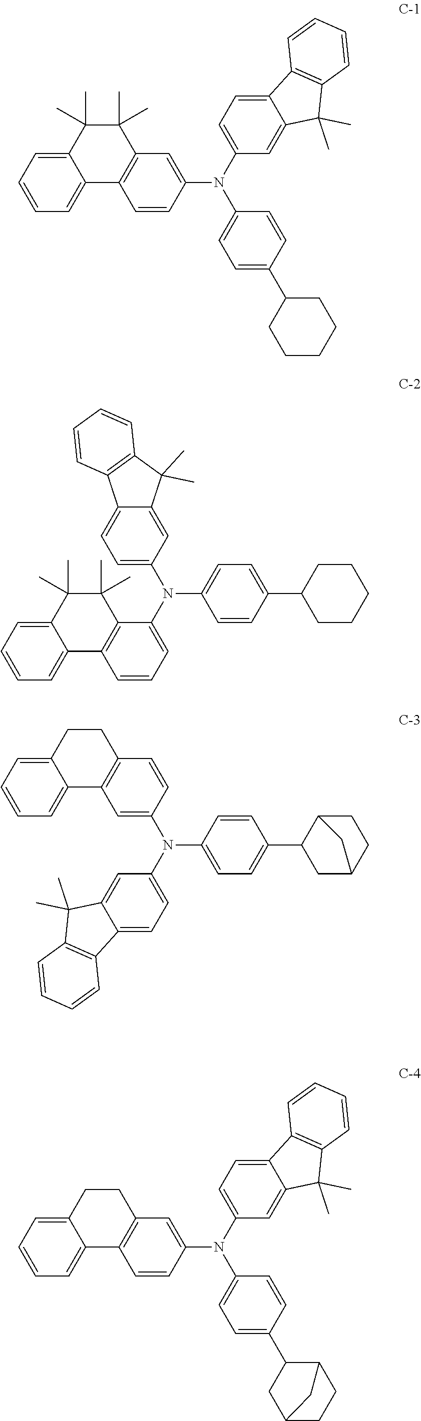 [SMILES: CC1(C)c2ccccc2-c2ccc(N(c3ccc(C4CCCCC4)cc3)c3ccc4c(c3)C(C)(C)C(C)(C)c3ccccc3-4)cc21 ; CC1(C)c2ccccc2-c2ccc(N(c3ccc(C4CCCCC4)cc3)c3cccc4c3C(C)(C)C(C)(C)c3ccccc3-4)cc21 ; CC1(C)c2ccccc2-c2ccc(N(c3ccc(C4CC5CCC4C5)cc3)c3ccc4c(c3)-c3ccccc3CC4)cc21 ; CC1(C)c2ccccc2-c2ccc(N(c3ccc(C4CC5CCC4C5)cc3)c3ccc4c(c3)CCc3ccccc3-4)cc21]

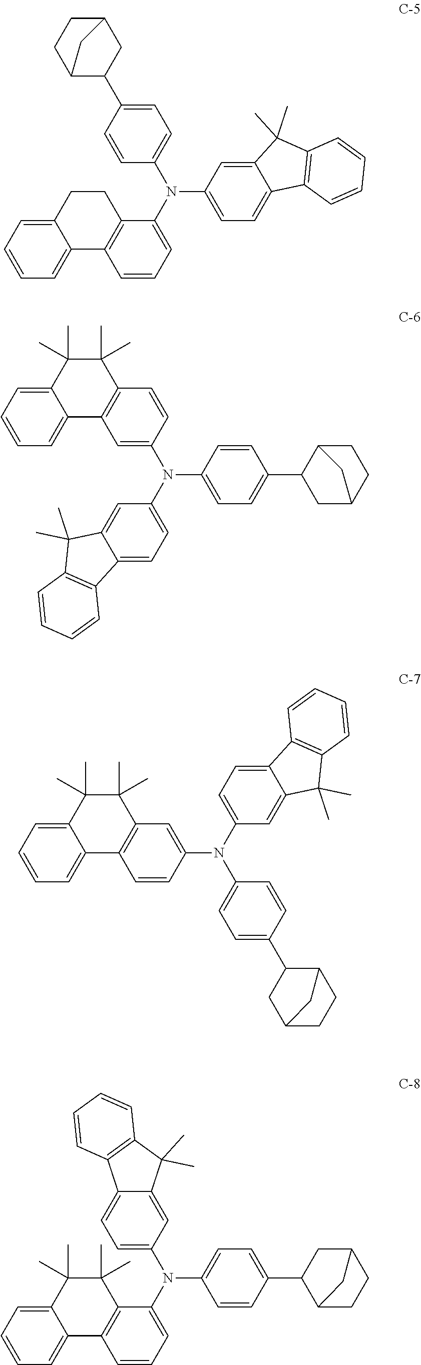 [SMILES: CC1(C)c2ccccc2-c2ccc(N(c3ccc(C4CC5CCC4C5)cc3)c3cccc4c3CCc3ccccc3-4)cc21 ; CC1(C)c2ccccc2-c2ccc(N(c3ccc(C4CC5CCC4C5)cc3)c3ccc4c(c3)-c3ccccc3C(C)(C)C4(C)C)cc21 ; CC1(C)c2ccccc2-c2ccc(N(c3ccc(C4CC5CCC4C5)cc3)c3ccc4c(c3)C(C)(C)C(C)(C)c3ccccc3-4)cc21 ; CC1(C)c2ccccc2-c2ccc(N(c3ccc(C4CC5CCC4C5)cc3)c3cccc4c3C(C)(C)C(C)(C)c3ccccc3-4)cc21]

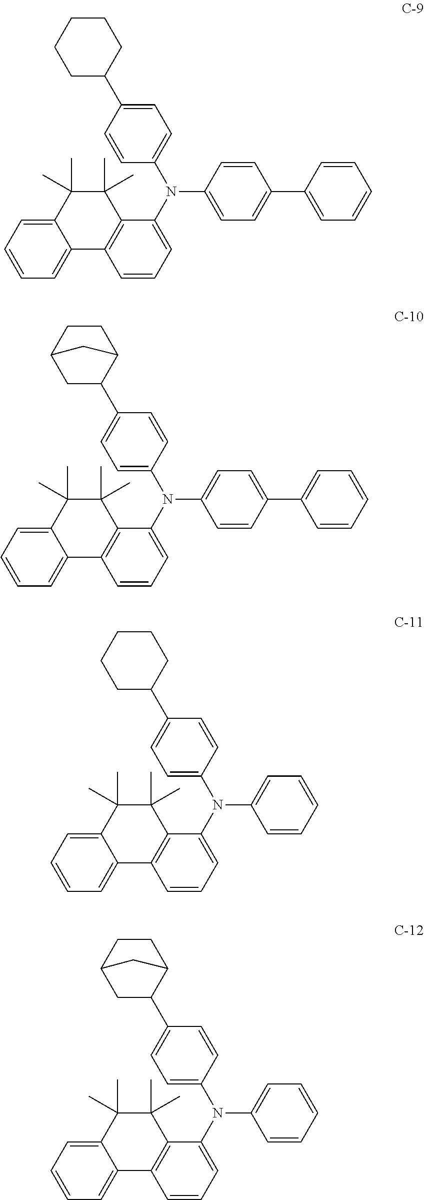

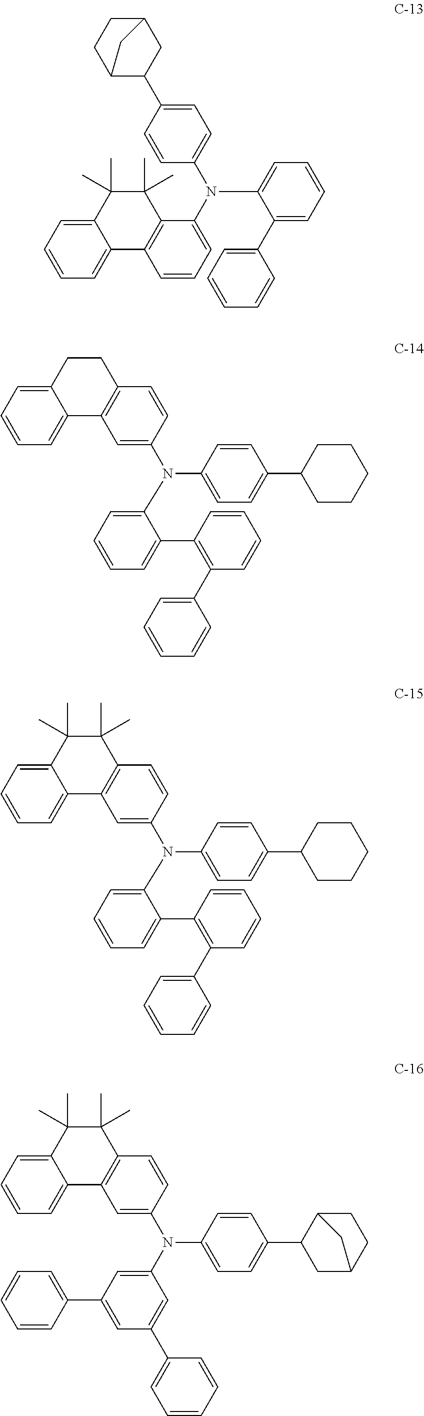 [SMILES: CC1(C)c2ccccc2-c2cccc(N(c3ccc(C4CC5CCC4C5)cc3)c3ccccc3-c3ccccc3)c2C1(C)C ; c1ccc(-c2ccccc2-c2ccccc2N(c2ccc(C3CCCCC3)cc2)c2ccc3c(c2)-c2ccccc2CC3)cc1 ; CC1(C)c2ccccc2-c2cc(N(c3ccc(C4CCCCC4)cc3)c3ccccc3-c3ccccc3-c3ccccc3)ccc2C1(C)C ; CC1(C)c2ccccc2-c2cc(N(c3ccc(C4CC5CCC4C5)cc3)c3cc(-c4ccccc4)cc(-c4ccccc4)c3)ccc2C1(C)C]

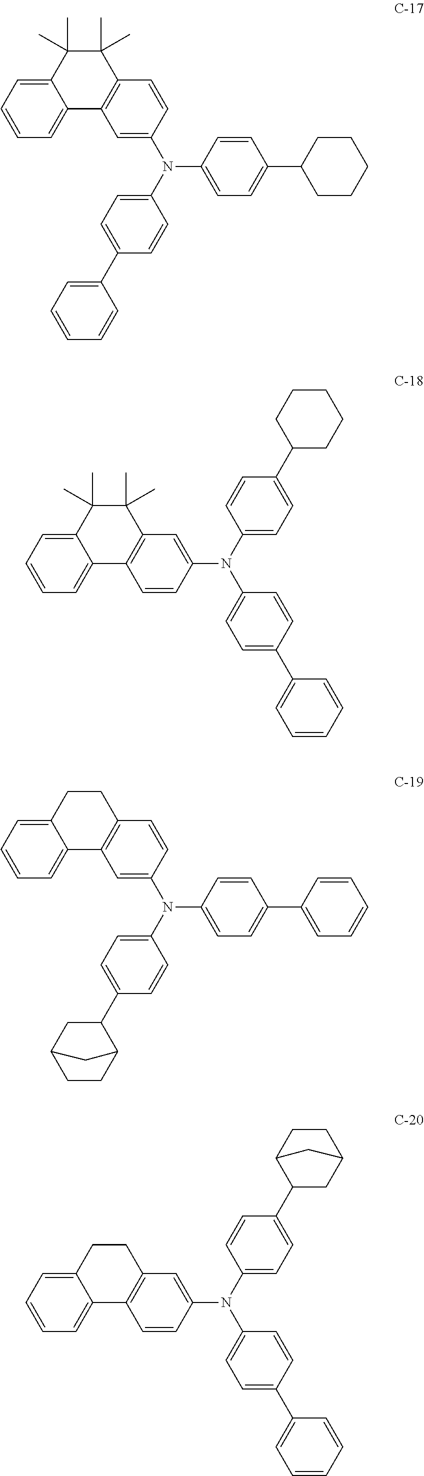 [SMILES: CC1(C)c2ccccc2-c2cc(N(c3ccc(-c4ccccc4)cc3)c3ccc(C4CCCCC4)cc3)ccc2C1(C)C ; CC1(C)c2ccccc2-c2ccc(N(c3ccc(-c4ccccc4)cc3)c3ccc(C4CCCCC4)cc3)cc2C1(C)C ; c1ccc(-c2ccc(N(c3ccc(C4CC5CCC4C5)cc3)c3ccc4c(c3)-c3ccccc3CC4)cc2)cc1 ; c1ccc(-c2ccc(N(c3ccc(C4CC5CCC4C5)cc3)c3ccc4c(c3)CCc3ccccc3-4)cc2)cc1]

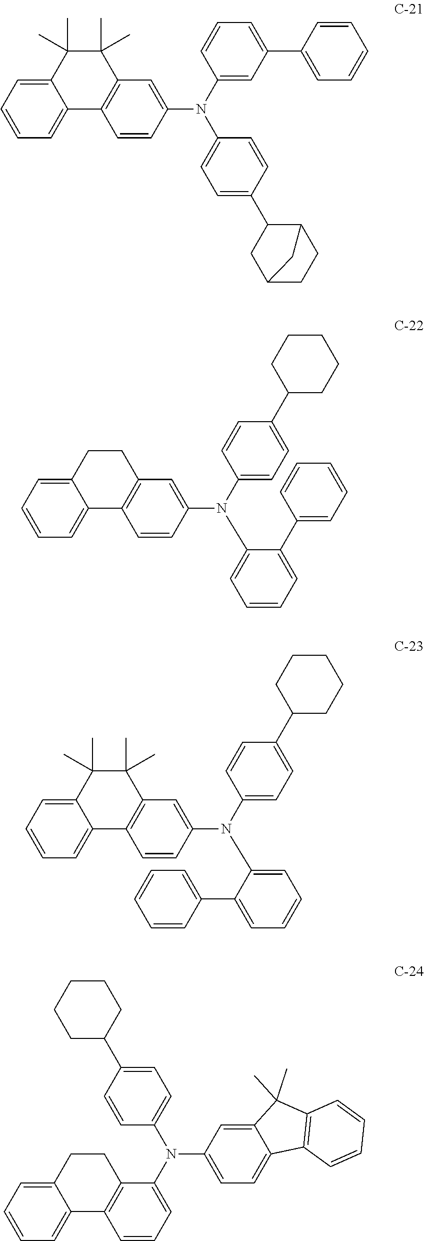 [SMILES: CC1(C)c2ccccc2-c2ccc(N(c3ccc(C4CC5CCC4C5)cc3)c3cccc(-c4ccccc4)c3)cc2C1(C)C ; c1ccc(-c2ccccc2N(c2ccc(C3CCCCC3)cc2)c2ccc3c(c2)CCc2ccccc2-3)cc1 ; CC1(C)c2ccccc2-c2ccc(N(c3ccc(C4CCCCC4)cc3)c3ccccc3-c3ccccc3)cc2C1(C)C ; CC1(C)c2ccccc2-c2ccc(N(c3ccc(C4CCCCC4)cc3)c3cccc4c3CCc3ccccc3-4)cc21]

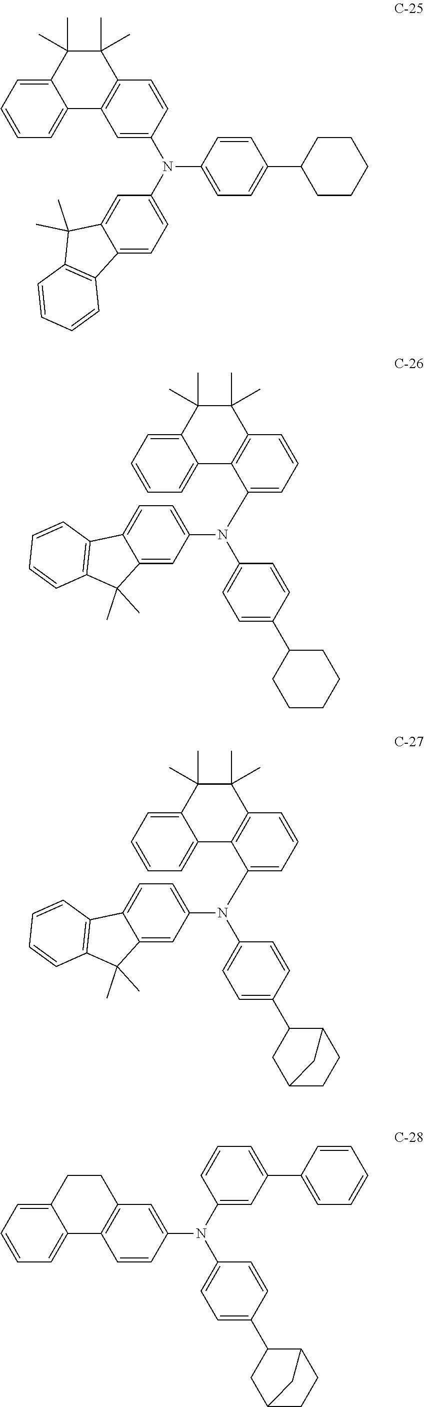 [SMILES: CC1(C)c2ccccc2-c2ccc(N(c3ccc(C4CCCCC4)cc3)c3ccc4c(c3)-c3ccccc3C(C)(C)C4(C)C)cc21 ; CC1(C)c2ccccc2-c2ccc(N(c3ccc(C4CCCCC4)cc3)c3cccc4c3-c3ccccc3C(C)(C)C4(C)C)cc21 ; CC1(C)c2ccccc2-c2ccc(N(c3ccc(C4CC5CCC4C5)cc3)c3cccc4c3-c3ccccc3C(C)(C)C4(C)C)cc21 ; c1ccc(-c2cccc(N(c3ccc(C4CC5CCC4C5)cc3)c3ccc4c(c3)CCc3ccccc3-4)c2)cc1]

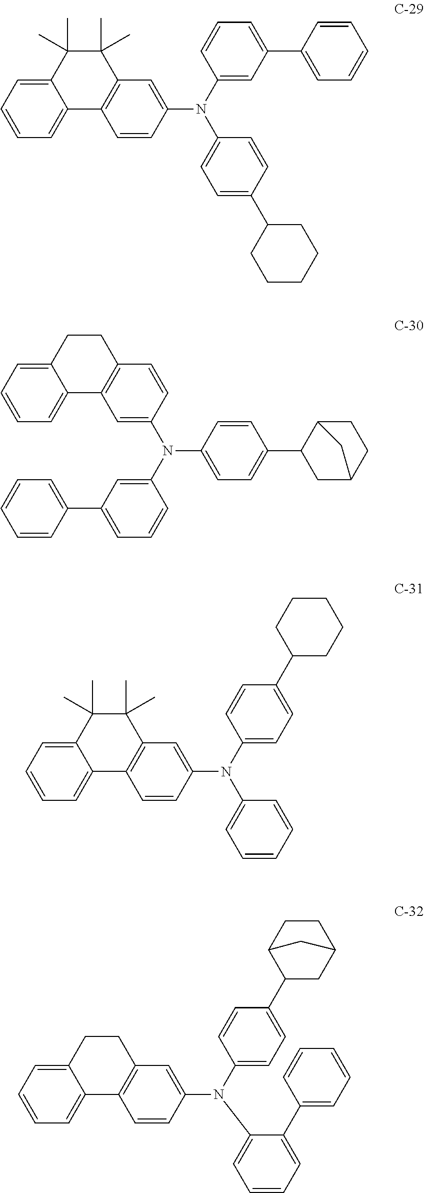 [SMILES: CC1(C)c2ccccc2-c2ccc(N(c3ccc(C4CCCCC4)cc3)c3cccc(-c4ccccc4)c3)cc2C1(C)C ; c1ccc(-c2cccc(N(c3ccc(C4CC5CCC4C5)cc3)c3ccc4c(c3)-c3ccccc3CC4)c2)cc1 ; CC1(C)c2ccccc2-c2ccc(N(c3ccccc3)c3ccc(C4CCCCC4)cc3)cc2C1(C)C ; c1ccc(-c2ccccc2N(c2ccc(C3CC4CCC3C4)cc2)c2ccc3c(c2)CCc2ccccc2-3)cc1]

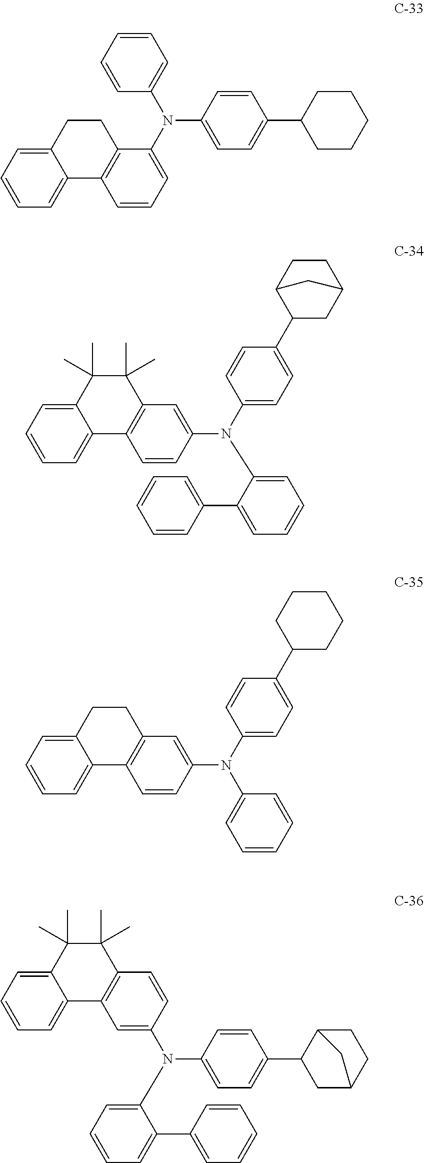 [SMILES: c1ccc(N(c2ccc(C3CCCCC3)cc2)c2cccc3c2CCc2ccccc2-3)cc1 ; CC1(C)c2ccccc2-c2ccc(N(c3ccc(C4CC5CCC4C5)cc3)c3ccccc3-c3ccccc3)cc2C1(C)C ; c1ccc(N(c2ccc(C3CCCCC3)cc2)c2ccc3c(c2)CCc2ccccc2-3)cc1 ; CC1(C)c2ccccc2-c2cc(N(c3ccc(C4CC5CCC4C5)cc3)c3ccccc3-c3ccccc3)ccc2C1(C)C]

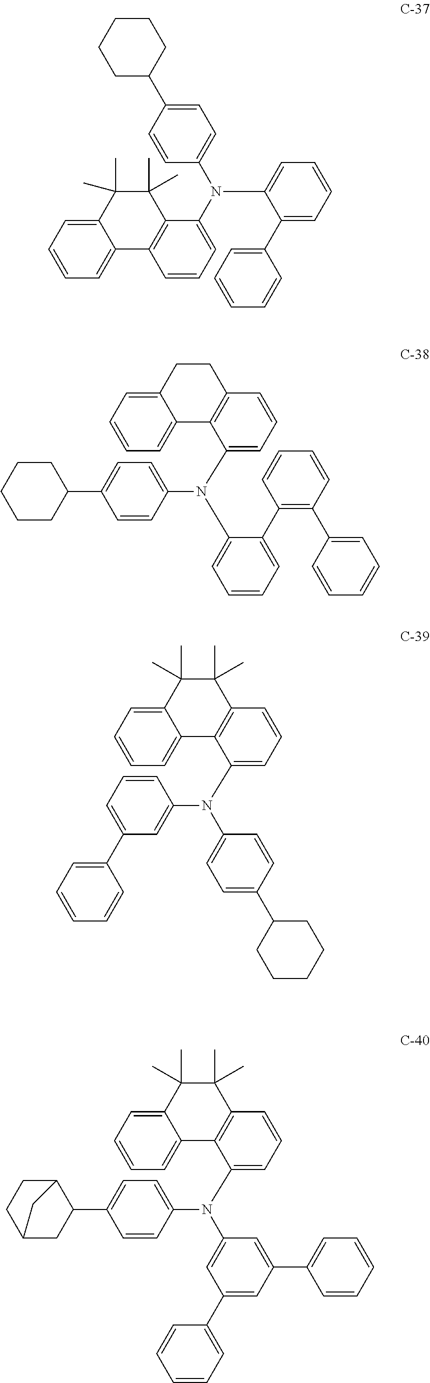 [SMILES: CC1(C)c2ccccc2-c2cccc(N(c3ccc(C4CCCCC4)cc3)c3ccccc3-c3ccccc3)c2C1(C)C ; c1ccc(-c2ccccc2-c2ccccc2N(c2ccc(C3CCCCC3)cc2)c2cccc3c2-c2ccccc2CC3)cc1 ; CC1(C)c2ccccc2-c2c(N(c3ccc(C4CCCCC4)cc3)c3cccc(-c4ccccc4)c3)cccc2C1(C)C ; CC1(C)c2ccccc2-c2c(N(c3ccc(C4CC5CCC4C5)cc3)c3cc(-c4ccccc4)cc(-c4ccccc4)c3)cccc2C1(C)C]

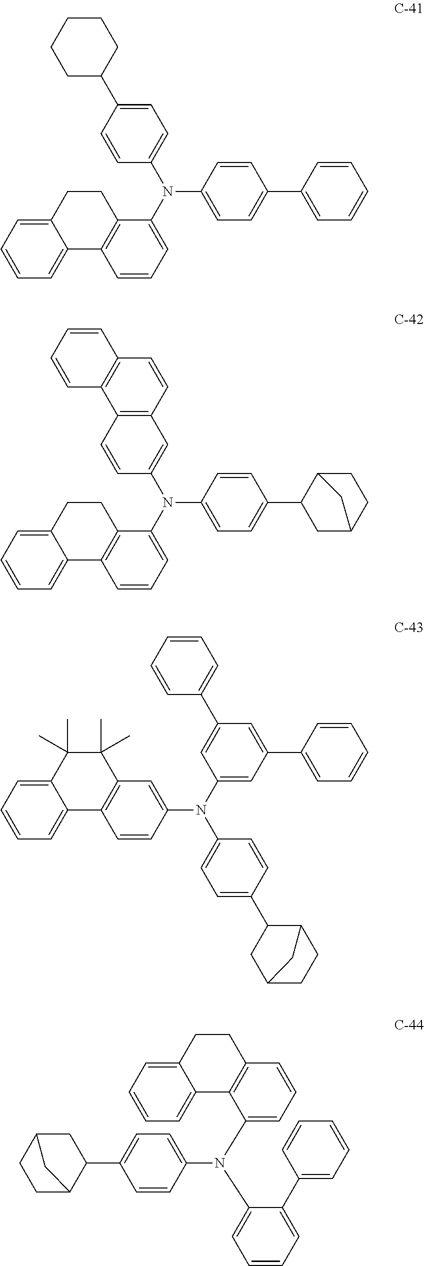 [SMILES: c1ccc(-c2ccc(N(c3ccc(C4CCCCC4)cc3)c3cccc4c3CCc3ccccc3-4)cc2)cc1 ; c1ccc2c(c1)CCc1c-2cccc1N(c1ccc(C2CC3CCC2C3)cc1)c1ccc2c(ccc3ccccc32)c1 ; CC1(C)c2ccccc2-c2ccc(N(c3ccc(C4CC5CCC4C5)cc3)c3cc(-c4ccccc4)cc(-c4ccccc4)c3)cc2C1(C)C ; c1ccc(-c2ccccc2N(c2ccc(C3CC4CCC3C4)cc2)c2cccc3c2-c2ccccc2CC3)cc1]

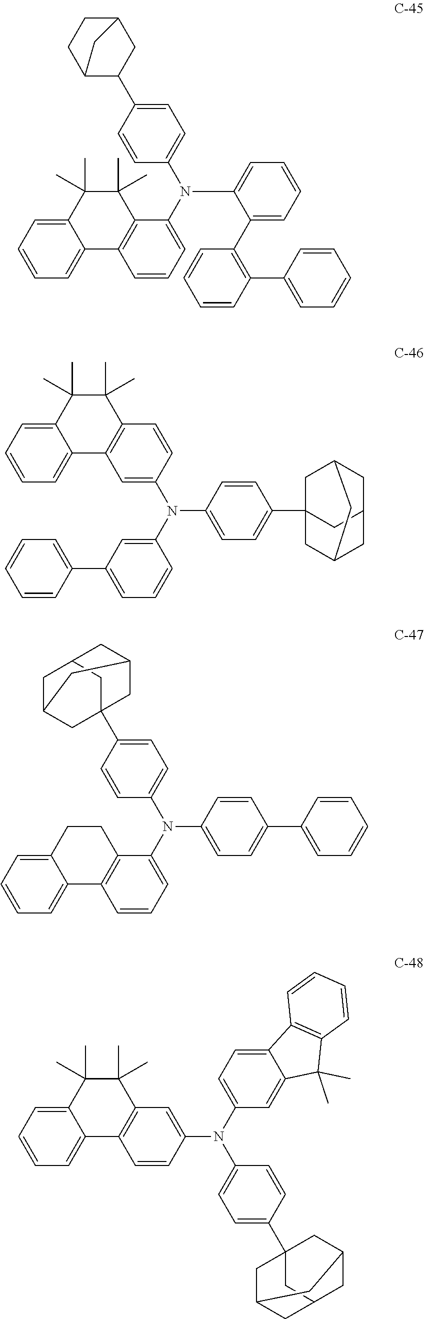 [SMILES: CC1(C)c2ccccc2-c2cccc(N(c3ccc(C4CC5CCC4C5)cc3)c3ccccc3-c3ccccc3-c3ccccc3)c2C1(C)C ; CC1(C)c2ccccc2-c2cc(N(c3ccc(C45CC6CC(CC(C6)C4)C5)cc3)c3cccc(-c4ccccc4)c3)ccc2C1(C)C ; c1ccc(-c2ccc(N(c3ccc(C45CC6CC(CC(C6)C4)C5)cc3)c3cccc4c3CCc3ccccc3-4)cc2)cc1 ; CC1(C)c2ccccc2-c2ccc(N(c3ccc(C45CC6CC(CC(C6)C4)C5)cc3)c3ccc4c(c3)C(C)(C)C(C)(C)c3ccccc3-4)cc21]

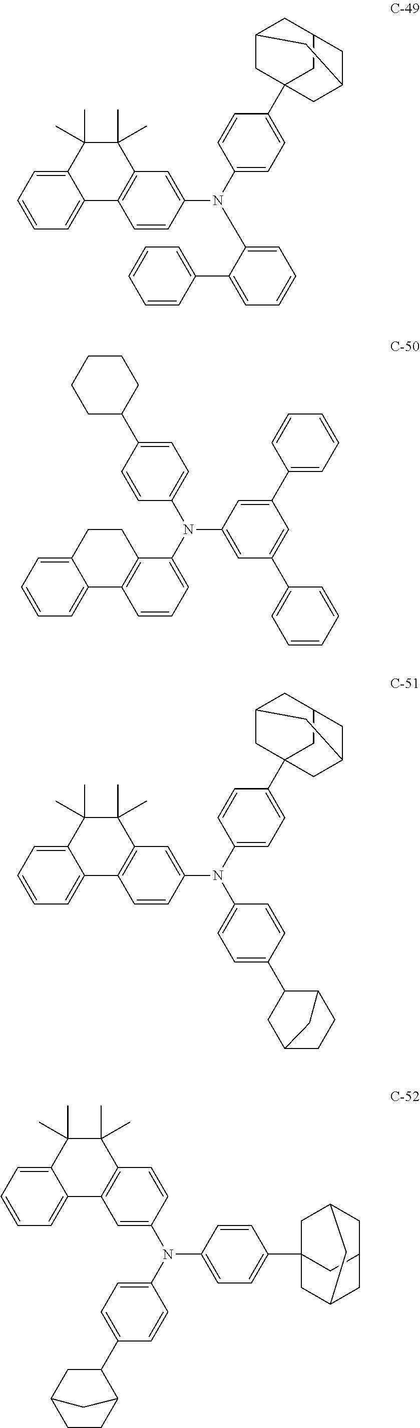 [SMILES: CC1(C)c2ccccc2-c2ccc(N(c3ccc(C45CC6CC(CC(C6)C4)C5)cc3)c3ccccc3-c3ccccc3)cc2C1(C)C ; c1ccc(-c2cc(-c3ccccc3)cc(N(c3ccc(C4CCCCC4)cc3)c3cccc4c3CCc3ccccc3-4)c2)cc1 ; CC1(C)c2ccccc2-c2ccc(N(c3ccc(C4CC5CCC4C5)cc3)c3ccc(C45CC6CC(CC(C6)C4)C5)cc3)cc2C1(C)C ; CC1(C)c2ccccc2-c2cc(N(c3ccc(C4CC5CCC4C5)cc3)c3ccc(C45CC6CC(CC(C6)C4)C5)cc3)ccc2C1(C)C]

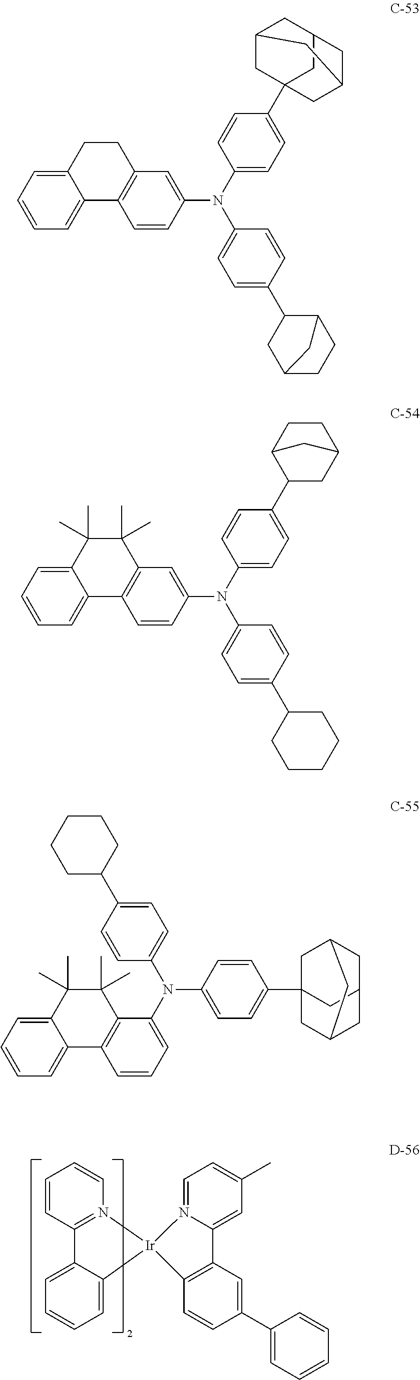

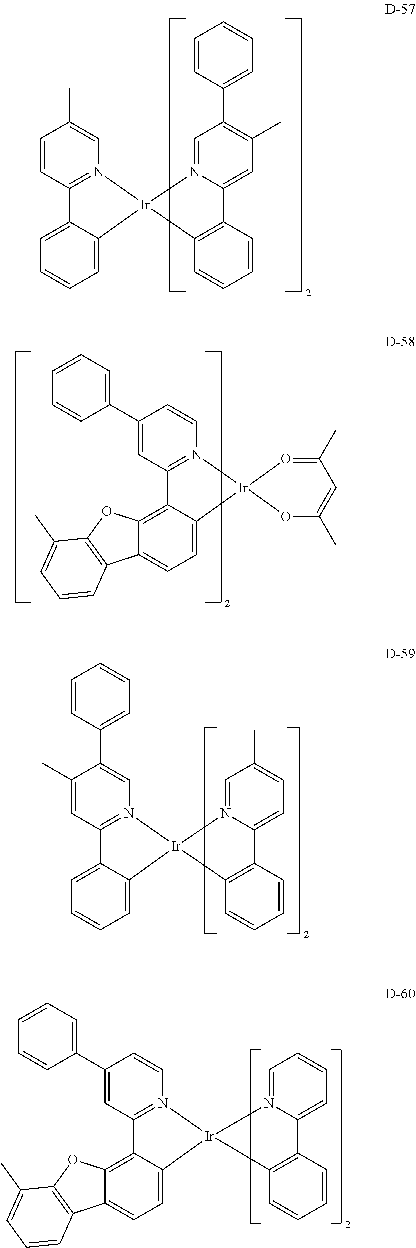

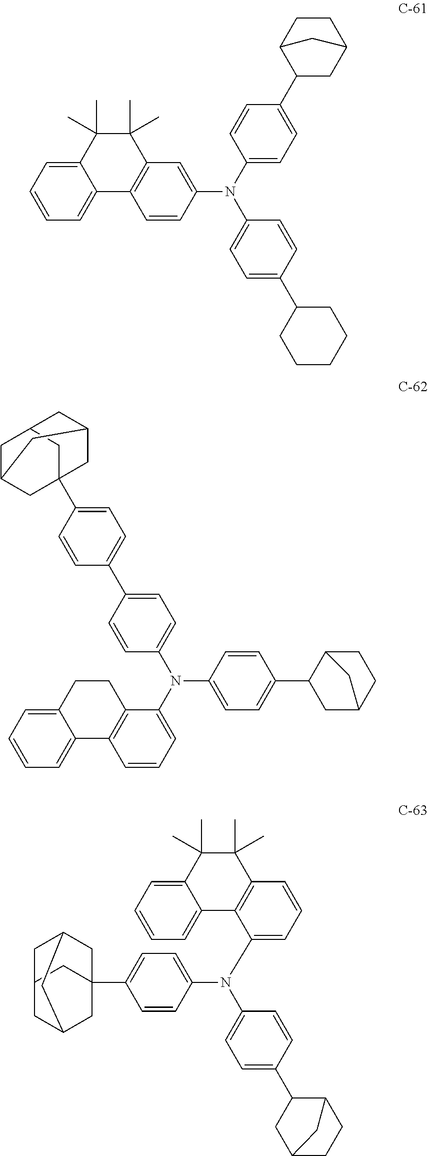 [SMILES: CC1(C)c2ccccc2-c2ccc(N(c3ccc(C4CCCCC4)cc3)c3ccc(C4CC5CCC4C5)cc3)cc2C1(C)C ; c1ccc2c(c1)CCc1c-2cccc1N(c1ccc(-c2ccc(C34CC5CC(CC(C5)C3)C4)cc2)cc1)c1ccc(C2CC3CCC2C3)cc1 ; CC1(C)c2ccccc2-c2c(N(c3ccc(C4CC5CCC4C5)cc3)c3ccc(C45CC6CC(CC(C6)C4)C5)cc3)cccc2C1(C)C]

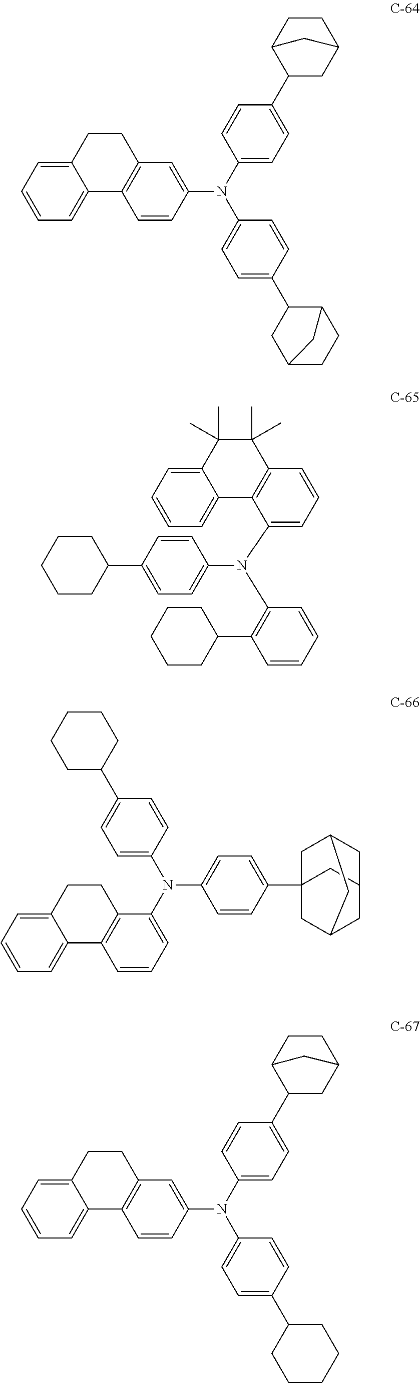 [SMILES: c1ccc2c(c1)CCc1cc(N(c3ccc(C4CC5CCC4C5)cc3)c3ccc(C4CC5CCC4C5)cc3)ccc1-2 ; CC1(C)c2ccccc2-c2c(N(c3ccc(C4CCCCC4)cc3)c3ccccc3C3CCCCC3)cccc2C1(C)C ; c1ccc2c(c1)CCc1c-2cccc1N(c1ccc(C2CCCCC2)cc1)c1ccc(C23CC4CC(CC(C4)C2)C3)cc1 ; c1ccc2c(c1)CCc1cc(N(c3ccc(C4CCCCC4)cc3)c3ccc(C4CC5CCC4C5)cc3)ccc1-2]

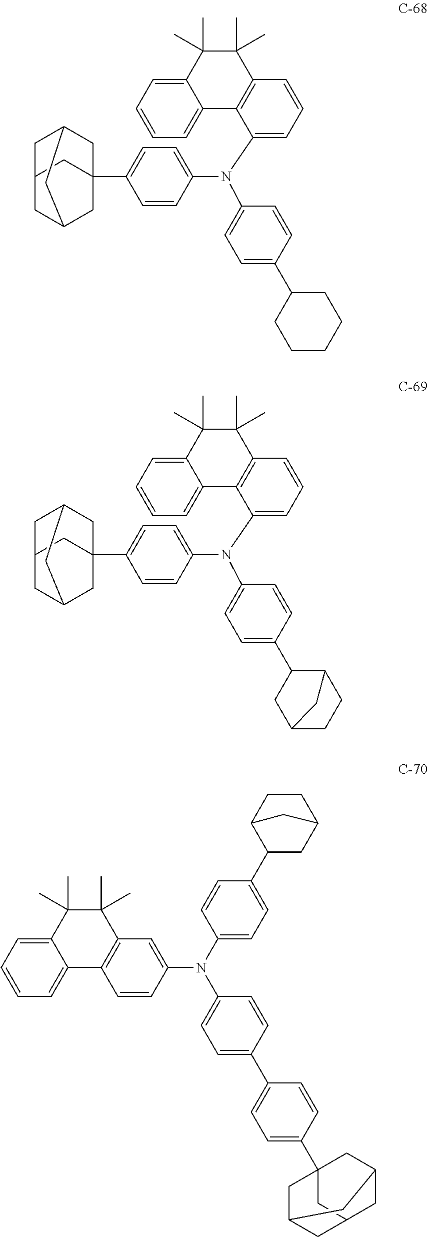 [SMILES: CC1(C)c2ccccc2-c2c(N(c3ccc(C4CCCCC4)cc3)c3ccc(C45CC6CC(CC(C6)C4)C5)cc3)cccc2C1(C)C ; CC1(C)c2ccccc2-c2c(N(c3ccc(C4CC5CCC4C5)cc3)c3ccc(C45CC6CC(CC(C6)C4)C5)cc3)cccc2C1(C)C ; CC1(C)c2ccccc2-c2ccc(N(c3ccc(-c4ccc(C56CC7CC(CC(C7)C5)C6)cc4)cc3)c3ccc(C4CC5CCC4C5)cc3)cc2C1(C)C]

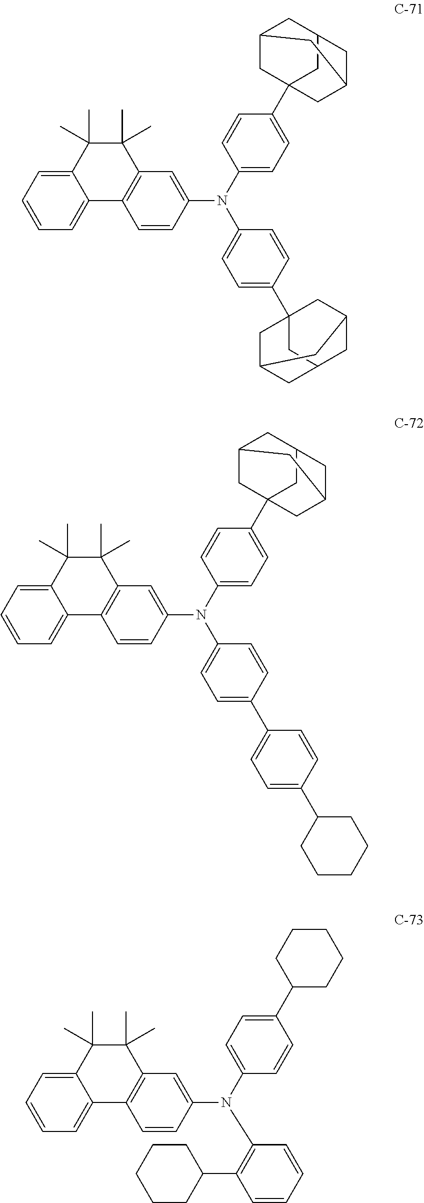 [SMILES: CC1(C)c2ccccc2-c2ccc(N(c3ccc(C45CC6CC(CC(C6)C4)C5)cc3)c3ccc(C45CC6CC(CC(C6)C4)C5)cc3)cc2C1(C)C ; CC1(C)c2ccccc2-c2ccc(N(c3ccc(-c4ccc(C5CCCCC5)cc4)cc3)c3ccc(C45CC6CC(CC(C6)C4)C5)cc3)cc2C1(C)C ; CC1(C)c2ccccc2-c2ccc(N(c3ccc(C4CCCCC4)cc3)c3ccccc3C3CCCCC3)cc2C1(C)C]

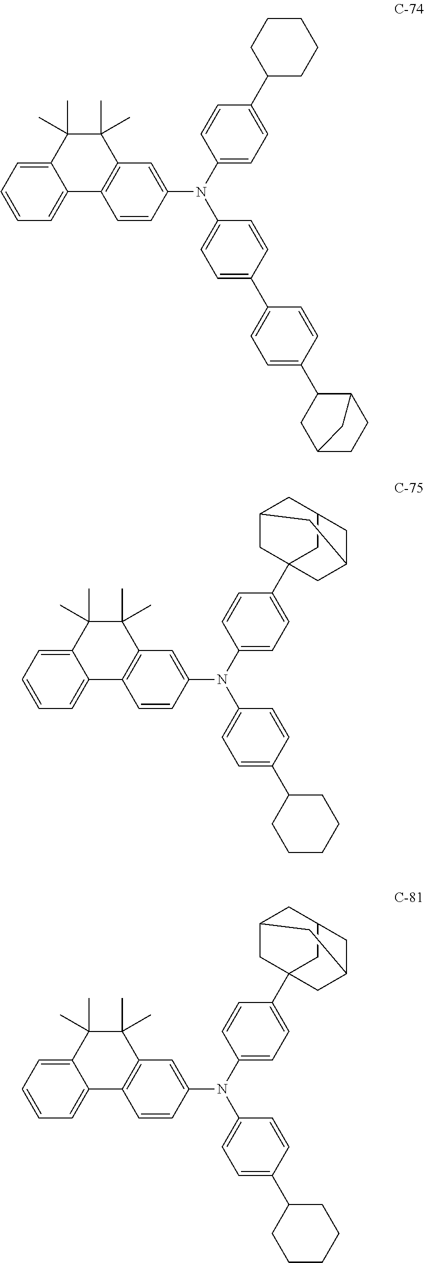 [SMILES: CC1(C)c2ccccc2-c2ccc(N(c3ccc(-c4ccc(C5CC6CCC5C6)cc4)cc3)c3ccc(C4CCCCC4)cc3)cc2C1(C)C ; CC1(C)c2ccccc2-c2ccc(N(c3ccc(C4CCCCC4)cc3)c3ccc(C45CC6CC(CC(C6)C4)C5)cc3)cc2C1(C)C ; CC1(C)c2ccccc2-c2ccc(N(c3ccc(C4CCCCC4)cc3)c3ccc(C45CC6CC(CC(C6)C4)C5)cc3)cc2C1(C)C]

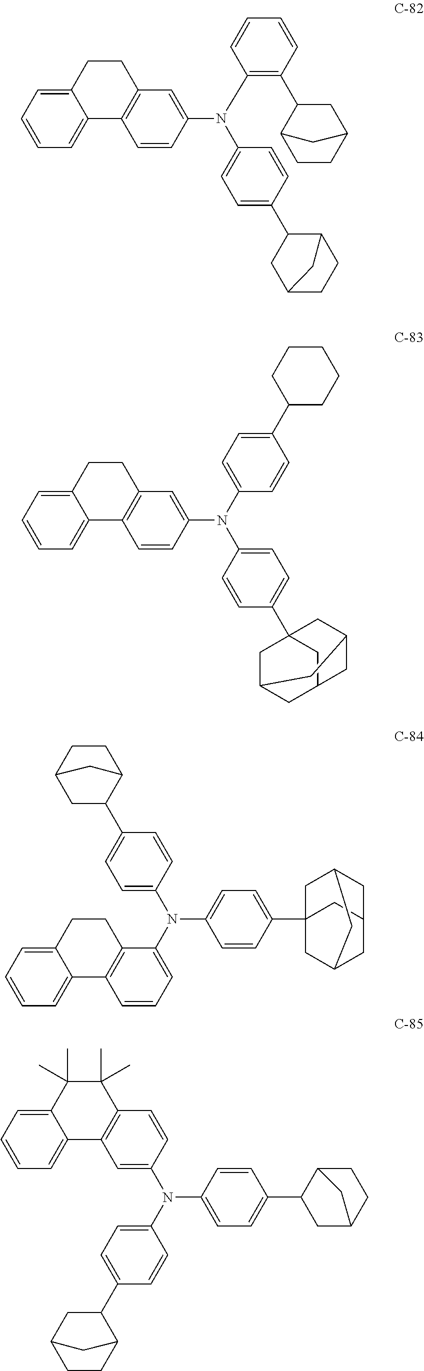

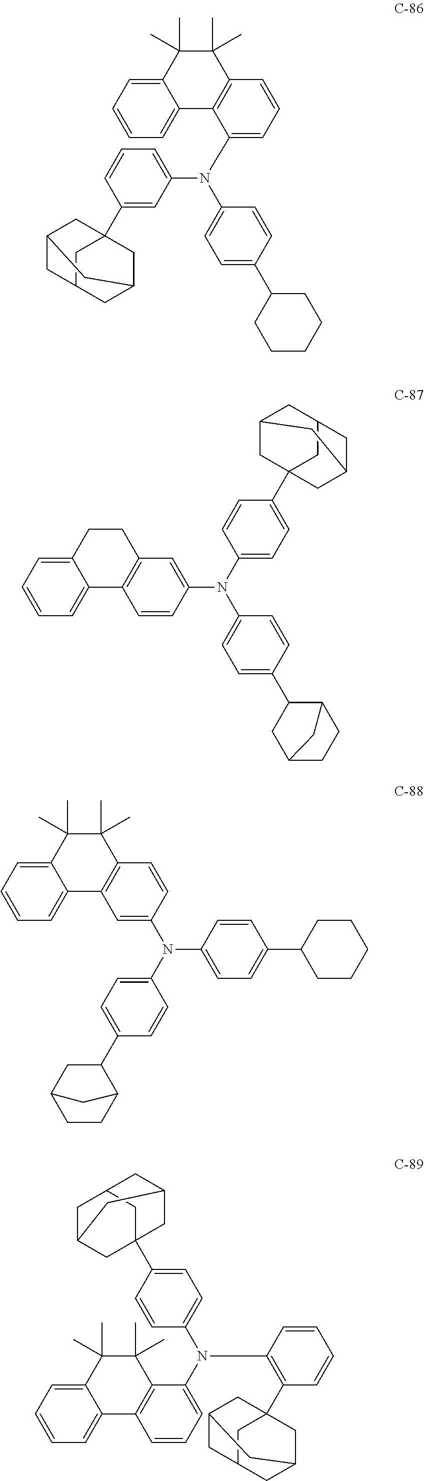 [SMILES: CC1(C)c2ccccc2-c2c(N(c3ccc(C4CCCCC4)cc3)c3cccc(C45CC6CC(CC(C6)C4)C5)c3)cccc2C1(C)C ; c1ccc2c(c1)CCc1cc(N(c3ccc(C4CC5CCC4C5)cc3)c3ccc(C45CC6CC(CC(C6)C4)C5)cc3)ccc1-2 ; CC1(C)c2ccccc2-c2cc(N(c3ccc(C4CCCCC4)cc3)c3ccc(C4CC5CCC4C5)cc3)ccc2C1(C)C ; CC1(C)c2ccccc2-c2cccc(N(c3ccc(C45CC6CC(CC(C6)C4)C5)cc3)c3ccccc3C34CC5CC(CC(C5)C3)C4)c2C1(C)C]

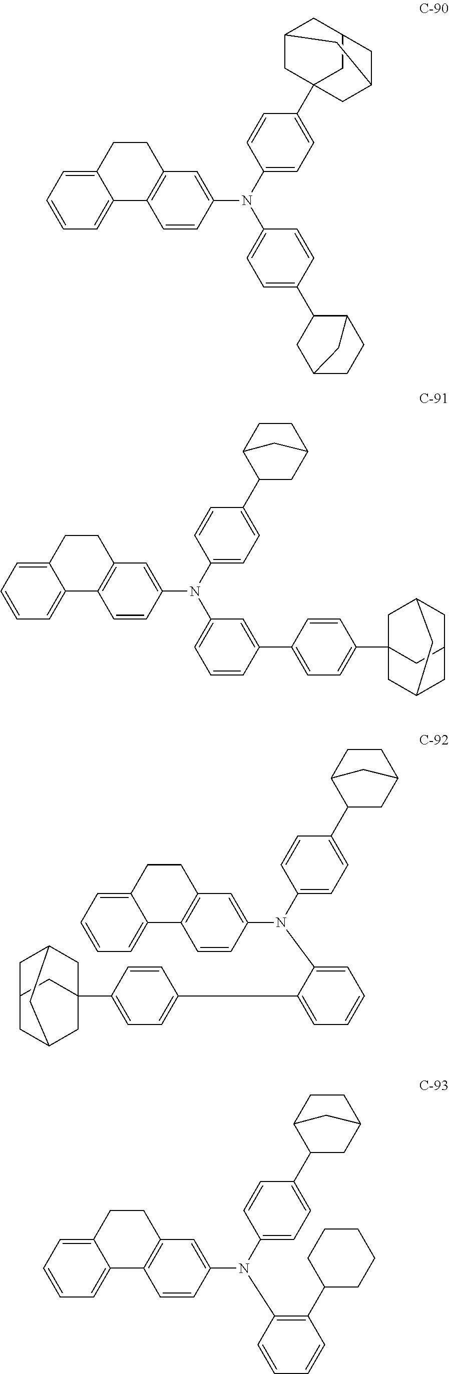 [SMILES: c1ccc2c(c1)CCc1cc(N(c3ccc(C4CC5CCC4C5)cc3)c3ccc(C45CC6CC(CC(C6)C4)C5)cc3)ccc1-2 ; c1cc(-c2ccc(C34CC5CC(CC(C5)C3)C4)cc2)cc(N(c2ccc(C3CC4CCC3C4)cc2)c2ccc3c(c2)CCc2ccccc2-3)c1 ; c1ccc2c(c1)CCc1cc(N(c3ccc(C4CC5CCC4C5)cc3)c3ccccc3-c3ccc(C45CC6CC(CC(C6)C4)C5)cc3)ccc1-2 ; c1ccc2c(c1)CCc1cc(N(c3ccc(C4CC5CCC4C5)cc3)c3ccccc3C3CCCCC3)ccc1-2]

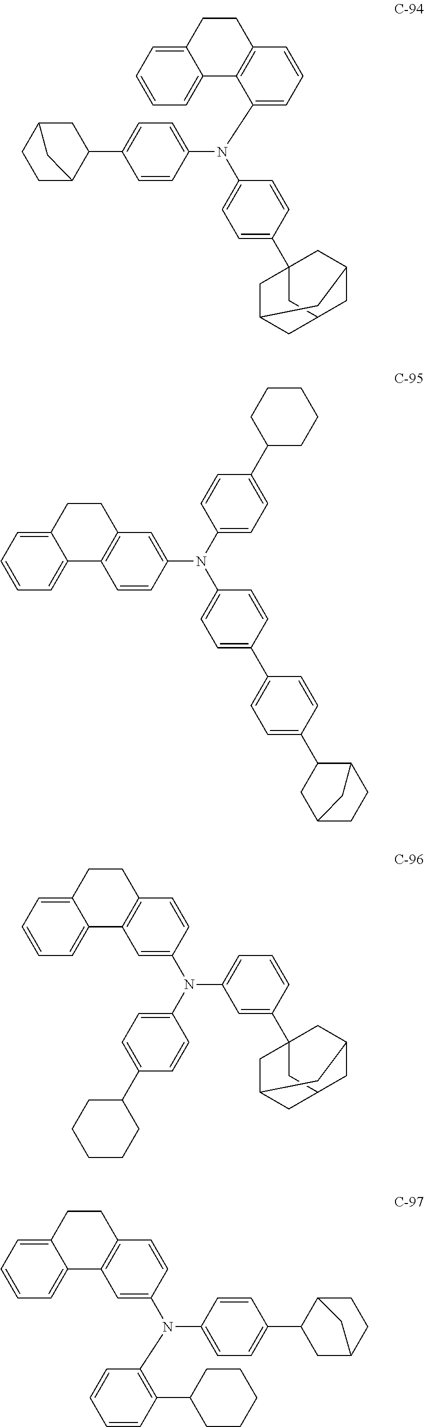 [SMILES: c1ccc2c(c1)CCc1cccc(N(c3ccc(C4CC5CCC4C5)cc3)c3ccc(C45CC6CC(CC(C6)C4)C5)cc3)c1-2 ; c1ccc2c(c1)CCc1cc(N(c3ccc(-c4ccc(C5CC6CCC5C6)cc4)cc3)c3ccc(C4CCCCC4)cc3)ccc1-2 ; c1cc(N(c2ccc(C3CCCCC3)cc2)c2ccc3c(c2)-c2ccccc2CC3)cc(C23CC4CC(CC(C4)C2)C3)c1 ; c1ccc2c(c1)CCc1ccc(N(c3ccc(C4CC5CCC4C5)cc3)c3ccccc3C3CCCCC3)cc1-2]

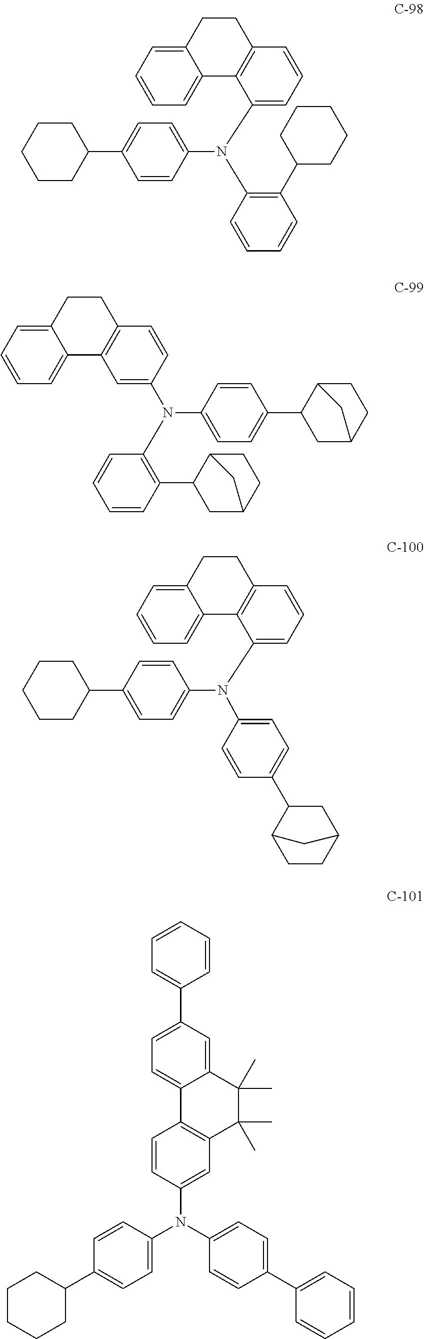

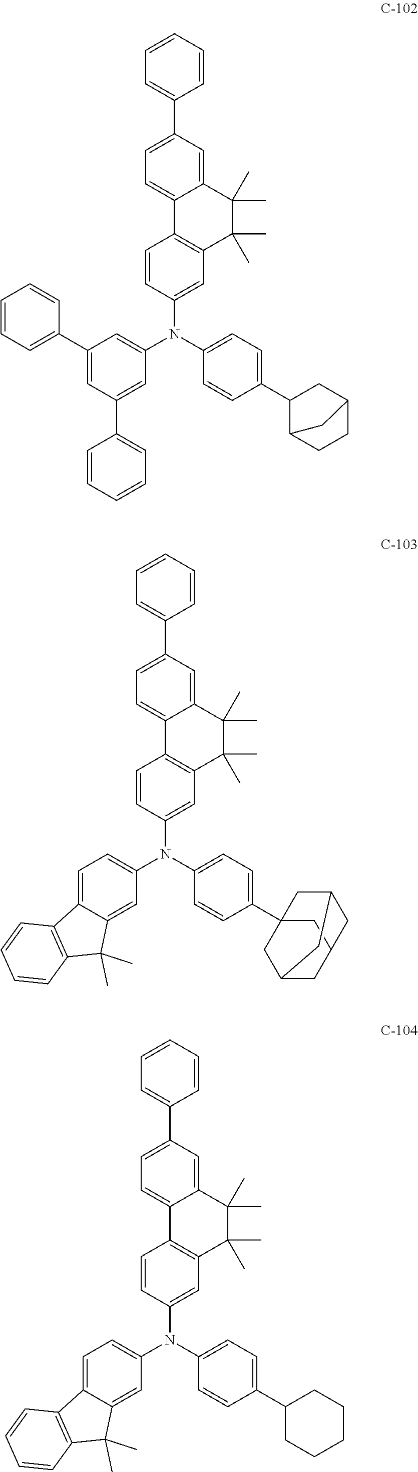 [SMILES: CC1(C)c2cc(-c3ccccc3)ccc2-c2ccc(N(c3ccc(C4CC5CCC4C5)cc3)c3cc(-c4ccccc4)cc(-c4ccccc4)c3)cc2C1(C)C ; CC1(C)c2ccccc2-c2ccc(N(c3ccc(C45CC6CC(CC(C6)C4)C5)cc3)c3ccc4c(c3)C(C)(C)C(C)(C)c3cc(-c5ccccc5)ccc3-4)cc21 ; CC1(C)c2ccccc2-c2ccc(N(c3ccc(C4CCCCC4)cc3)c3ccc4c(c3)C(C)(C)C(C)(C)c3cc(-c5ccccc5)ccc3-4)cc21]

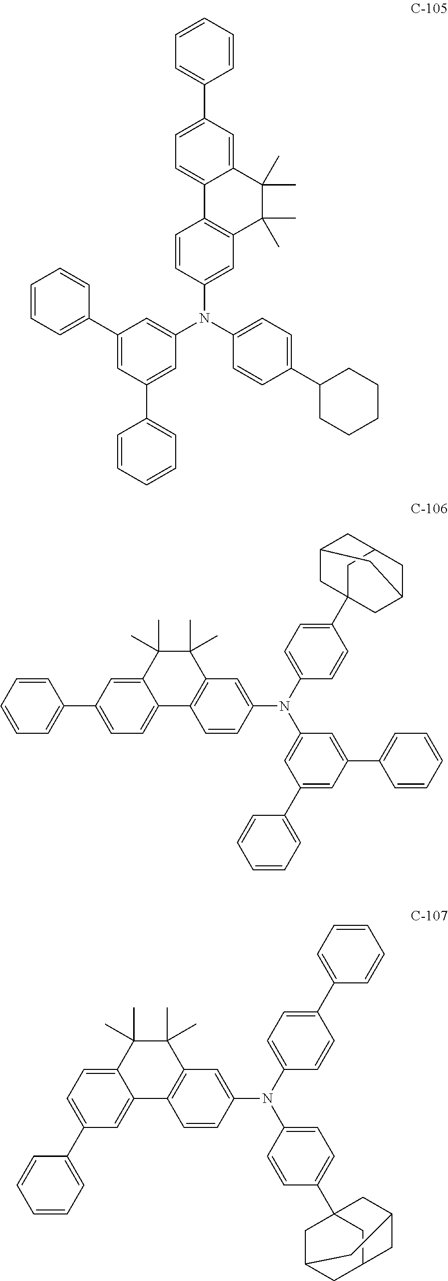 [SMILES: CC1(C)c2cc(-c3ccccc3)ccc2-c2ccc(N(c3ccc(C4CCCCC4)cc3)c3cc(-c4ccccc4)cc(-c4ccccc4)c3)cc2C1(C)C ; CC1(C)c2cc(-c3ccccc3)ccc2-c2ccc(N(c3ccc(C45CC6CC(CC(C6)C4)C5)cc3)c3cc(-c4ccccc4)cc(-c4ccccc4)c3)cc2C1(C)C ; CC1(C)c2ccc(-c3ccccc3)cc2-c2ccc(N(c3ccc(-c4ccccc4)cc3)c3ccc(C45CC6CC(CC(C6)C4)C5)cc3)cc2C1(C)C]

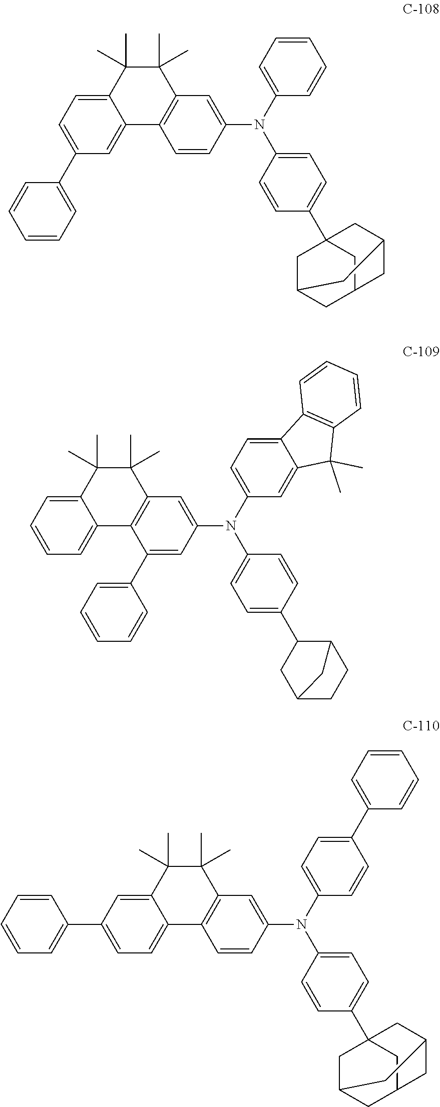 [SMILES: CC1(C)c2ccc(-c3ccccc3)cc2-c2ccc(N(c3ccccc3)c3ccc(C45CC6CC(CC(C6)C4)C5)cc3)cc2C1(C)C ; CC1(C)c2ccccc2-c2ccc(N(c3ccc(C4CC5CCC4C5)cc3)c3cc(-c4ccccc4)c4c(c3)C(C)(C)C(C)(C)c3ccccc3-4)cc21 ; CC1(C)c2cc(-c3ccccc3)ccc2-c2ccc(N(c3ccc(-c4ccccc4)cc3)c3ccc(C45CC6CC(CC(C6)C4)C5)cc3)cc2C1(C)C]

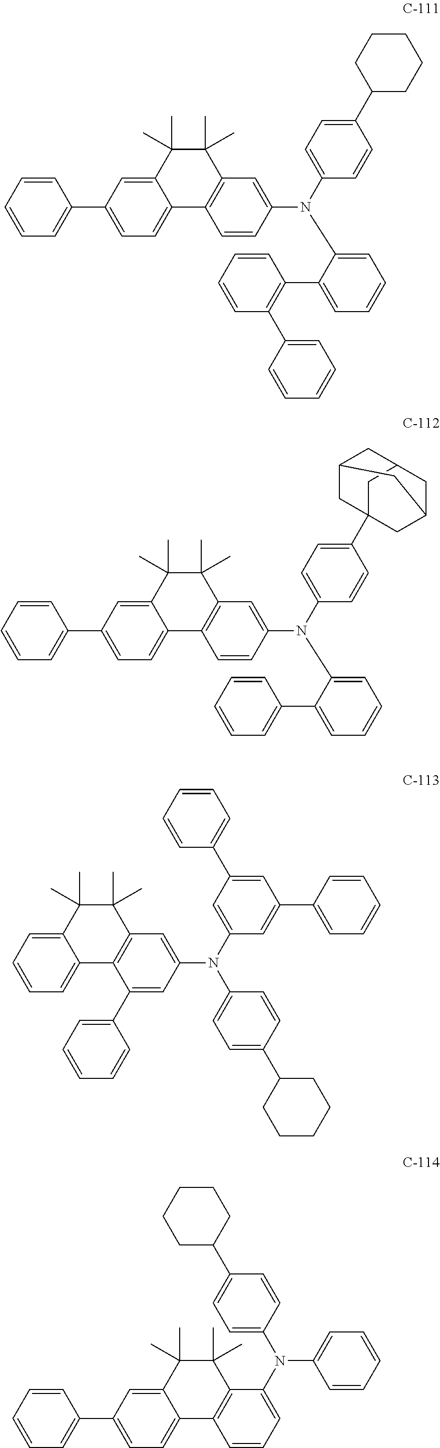 [SMILES: CC1(C)c2cc(-c3ccccc3)ccc2-c2ccc(N(c3ccc(C4CCCCC4)cc3)c3ccccc3-c3ccccc3-c3ccccc3)cc2C1(C)C ; CC1(C)c2cc(-c3ccccc3)ccc2-c2ccc(N(c3ccc(C45CC6CC(CC(C6)C4)C5)cc3)c3ccccc3-c3ccccc3)cc2C1(C)C ; CC1(C)c2ccccc2-c2c(-c3ccccc3)cc(N(c3ccc(C4CCCCC4)cc3)c3cc(-c4ccccc4)cc(-c4ccccc4)c3)cc2C1(C)C ; CC1(C)c2cc(-c3ccccc3)ccc2-c2cccc(N(c3ccccc3)c3ccc(C4CCCCC4)cc3)c2C1(C)C]

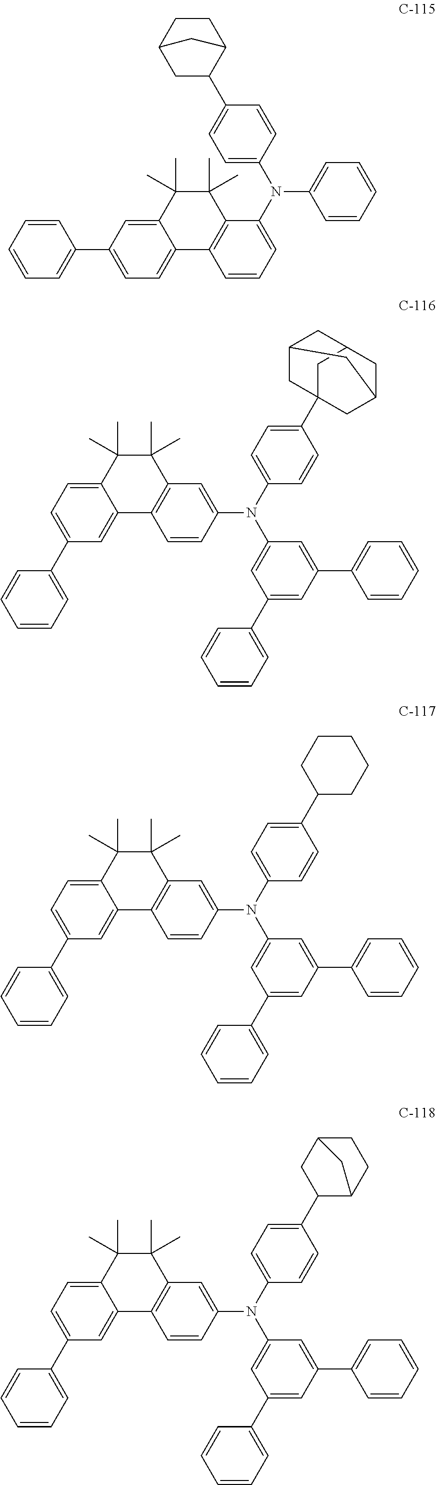 [SMILES: CC1(C)c2cc(-c3ccccc3)ccc2-c2cccc(N(c3ccccc3)c3ccc(C4CC5CCC4C5)cc3)c2C1(C)C ; CC1(C)c2ccc(-c3ccccc3)cc2-c2ccc(N(c3ccc(C45CC6CC(CC(C6)C4)C5)cc3)c3cc(-c4ccccc4)cc(-c4ccccc4)c3)cc2C1(C)C ; CC1(C)c2ccc(-c3ccccc3)cc2-c2ccc(N(c3ccc(C4CCCCC4)cc3)c3cc(-c4ccccc4)cc(-c4ccccc4)c3)cc2C1(C)C ; CC1(C)c2ccc(-c3ccccc3)cc2-c2ccc(N(c3ccc(C4CC5CCC4C5)cc3)c3cc(-c4ccccc4)cc(-c4ccccc4)c3)cc2C1(C)C]

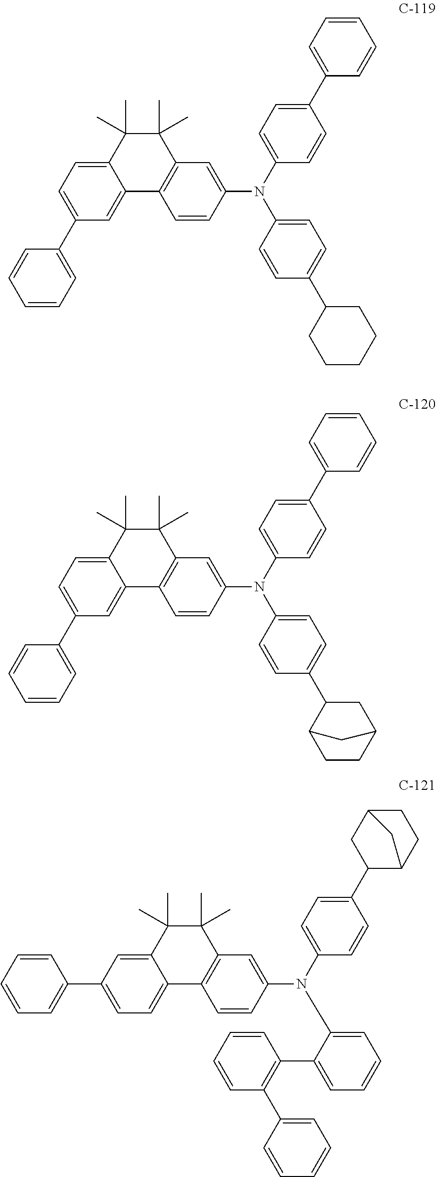 [SMILES: CC1(C)c2ccc(-c3ccccc3)cc2-c2ccc(N(c3ccc(-c4ccccc4)cc3)c3ccc(C4CCCCC4)cc3)cc2C1(C)C ; CC1(C)c2ccc(-c3ccccc3)cc2-c2ccc(N(c3ccc(-c4ccccc4)cc3)c3ccc(C4CC5CCC4C5)cc3)cc2C1(C)C ; CC1(C)c2cc(-c3ccccc3)ccc2-c2ccc(N(c3ccc(C4CC5CCC4C5)cc3)c3ccccc3-c3ccccc3-c3ccccc3)cc2C1(C)C]

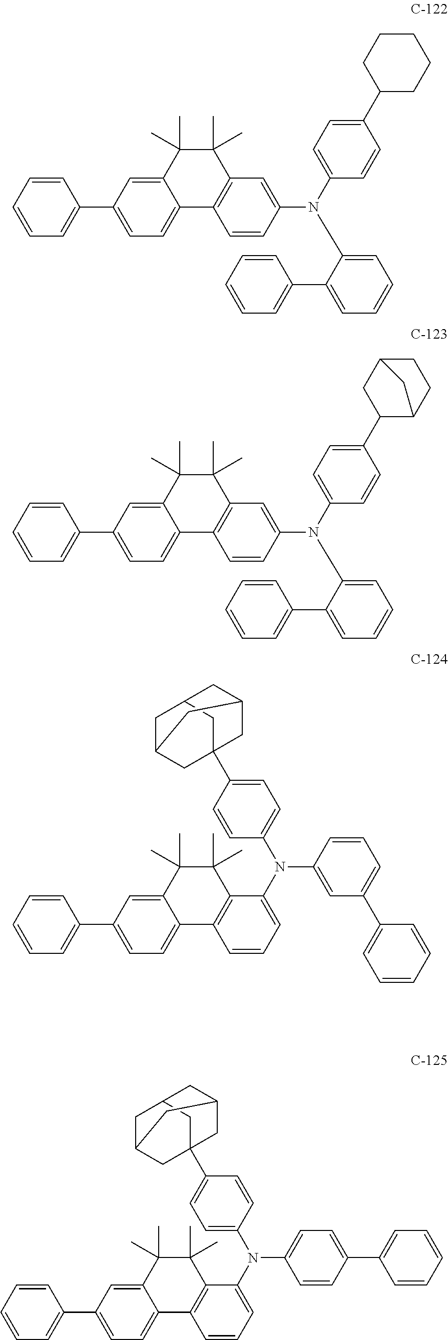

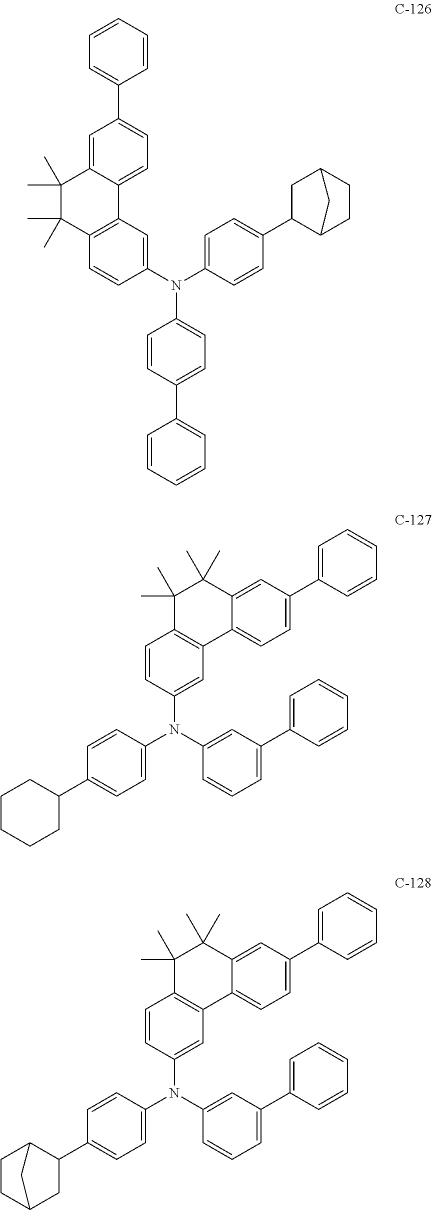 [SMILES: CC1(C)c2ccc(N(c3ccc(-c4ccccc4)cc3)c3ccc(C4CC5CCC4C5)cc3)cc2-c2ccc(-c3ccccc3)cc2C1(C)C ; CC1(C)c2ccc(N(c3ccc(C4CCCCC4)cc3)c3cccc(-c4ccccc4)c3)cc2-c2ccc(-c3ccccc3)cc2C1(C)C ; CC1(C)c2ccc(N(c3ccc(C4CC5CCC4C5)cc3)c3cccc(-c4ccccc4)c3)cc2-c2ccc(-c3ccccc3)cc2C1(C)C]

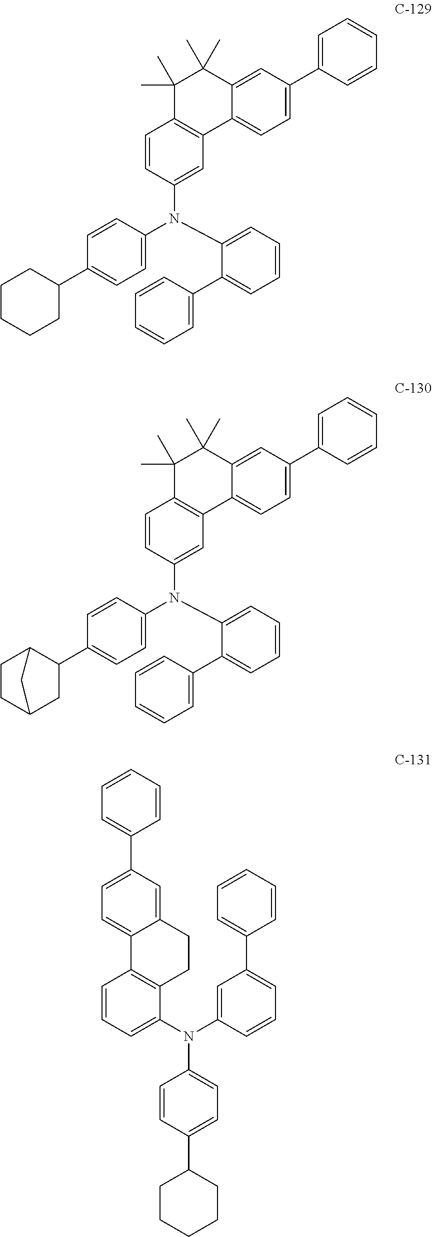 [SMILES: CC1(C)c2ccc(N(c3ccc(C4CCCCC4)cc3)c3ccccc3-c3ccccc3)cc2-c2ccc(-c3ccccc3)cc2C1(C)C ; CC1(C)c2ccc(N(c3ccc(C4CC5CCC4C5)cc3)c3ccccc3-c3ccccc3)cc2-c2ccc(-c3ccccc3)cc2C1(C)C ; c1ccc(-c2cccc(N(c3ccc(C4CCCCC4)cc3)c3cccc4c3CCc3cc(-c5ccccc5)ccc3-4)c2)cc1]

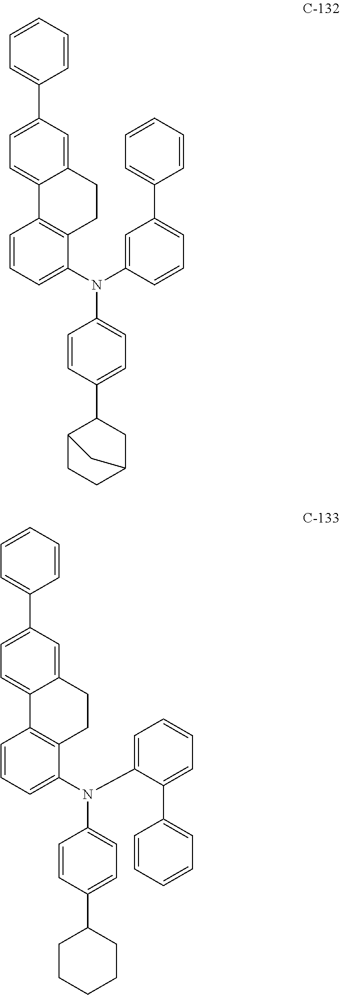 [SMILES: c1ccc(-c2cccc(N(c3ccc(C4CC5CCC4C5)cc3)c3cccc4c3CCc3cc(-c5ccccc5)ccc3-4)c2)cc1 ; c1ccc(-c2ccc3c(c2)CCc2c-3cccc2N(c2ccc(C3CCCCC3)cc2)c2ccccc2-c2ccccc2)cc1]

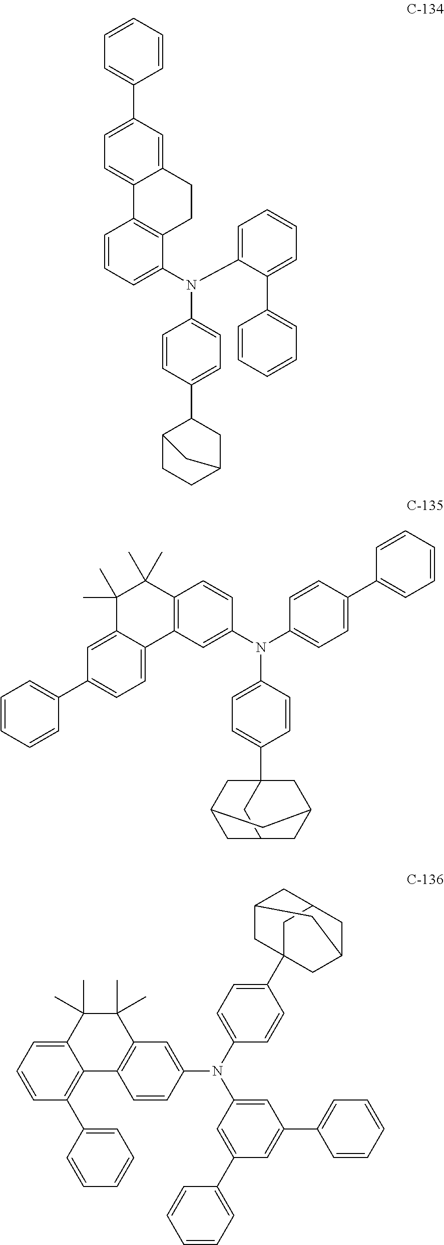 [SMILES: c1ccc(-c2ccc3c(c2)CCc2c-3cccc2N(c2ccc(C3CC4CCC3C4)cc2)c2ccccc2-c2ccccc2)cc1 ; CC1(C)c2ccc(N(c3ccc(-c4ccccc4)cc3)c3ccc(C45CC6CC(CC(C6)C4)C5)cc3)cc2-c2ccc(-c3ccccc3)cc2C1(C)C ; CC1(C)c2cc(N(c3ccc(C45CC6CC(CC(C6)C4)C5)cc3)c3cc(-c4ccccc4)cc(-c4ccccc4)c3)ccc2-c2c(-c3ccccc3)cccc2C1(C)C]

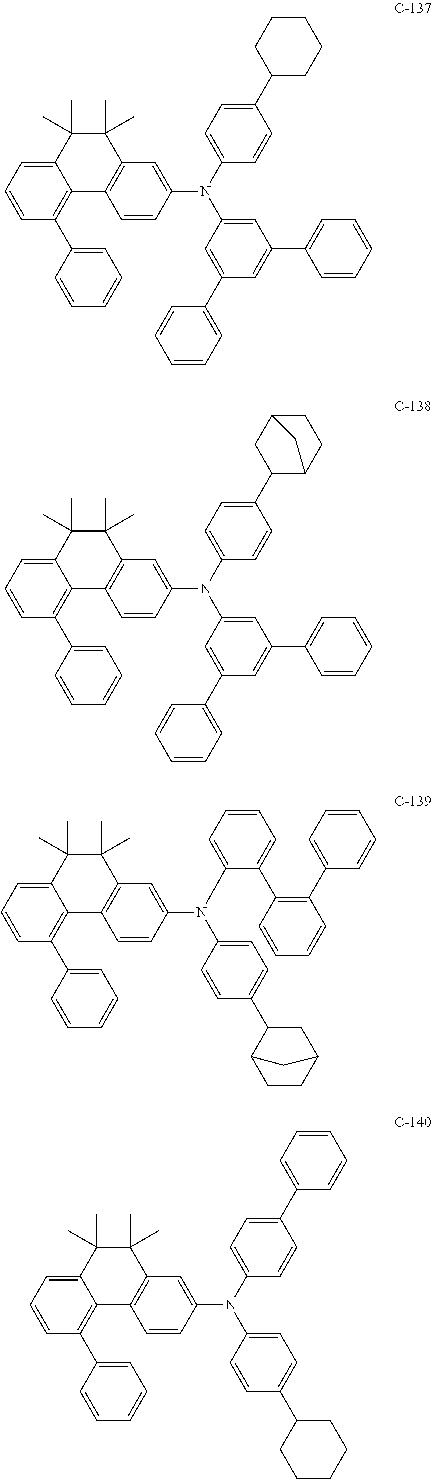

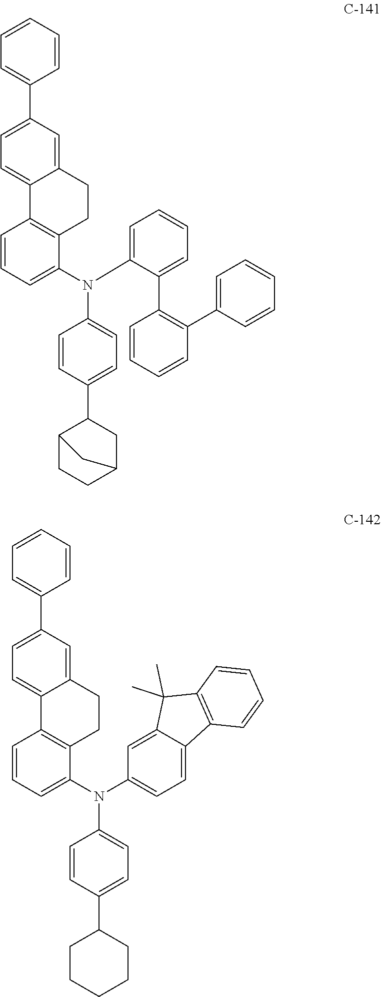 [SMILES: c1ccc(-c2ccc3c(c2)CCc2c-3cccc2N(c2ccc(C3CC4CCC3C4)cc2)c2ccccc2-c2ccccc2-c2ccccc2)cc1 ; CC1(C)c2ccccc2-c2ccc(N(c3ccc(C4CCCCC4)cc3)c3cccc4c3CCc3cc(-c5ccccc5)ccc3-4)cc21]

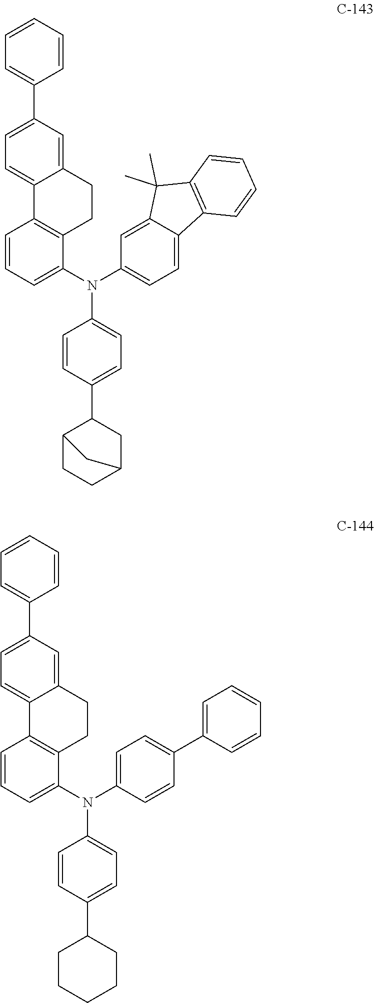 [SMILES: CC1(C)c2ccccc2-c2ccc(N(c3ccc(C4CC5CCC4C5)cc3)c3cccc4c3CCc3cc(-c5ccccc5)ccc3-4)cc21 ; c1ccc(-c2ccc(N(c3ccc(C4CCCCC4)cc3)c3cccc4c3CCc3cc(-c5ccccc5)ccc3-4)cc2)cc1]

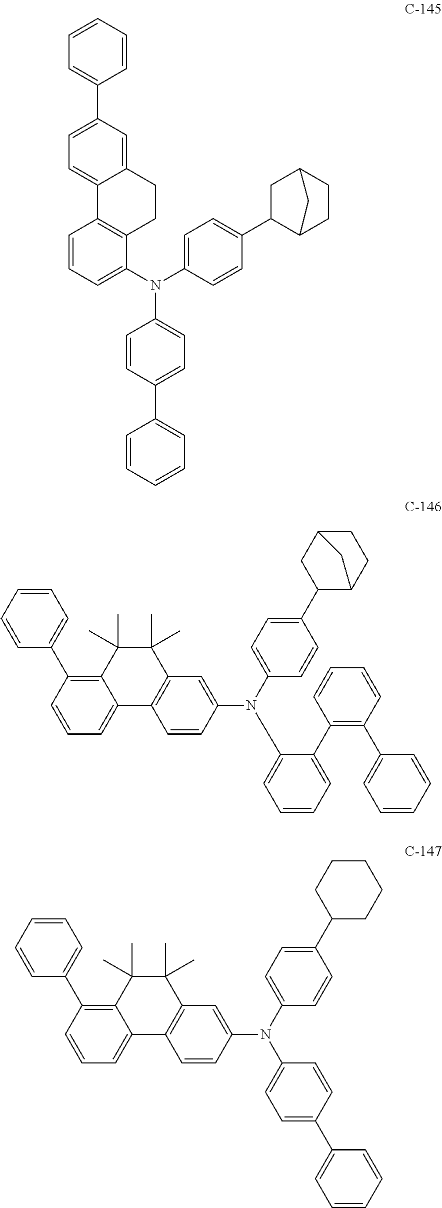 [SMILES: c1ccc(-c2ccc(N(c3ccc(C4CC5CCC4C5)cc3)c3cccc4c3CCc3cc(-c5ccccc5)ccc3-4)cc2)cc1 ; CC1(C)c2cc(N(c3ccc(C4CC5CCC4C5)cc3)c3ccccc3-c3ccccc3-c3ccccc3)ccc2-c2cccc(-c3ccccc3)c2C1(C)C ; CC1(C)c2cc(N(c3ccc(-c4ccccc4)cc3)c3ccc(C4CCCCC4)cc3)ccc2-c2cccc(-c3ccccc3)c2C1(C)C]

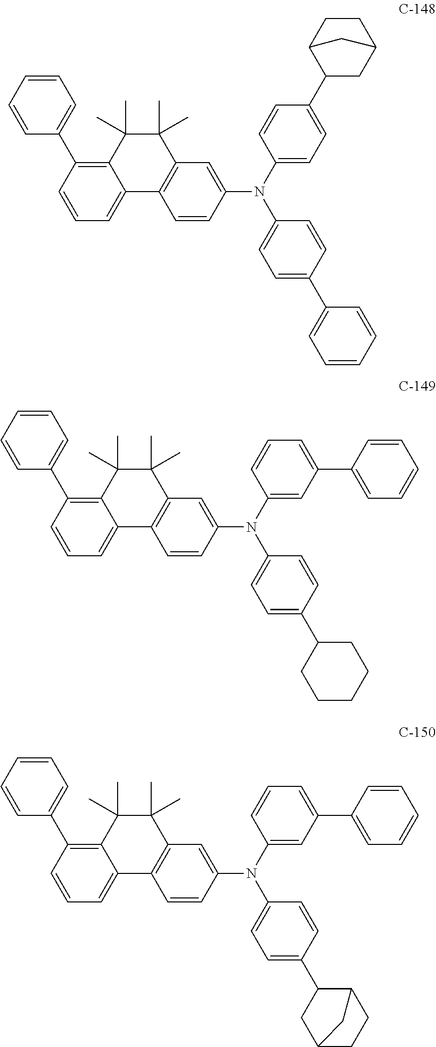 [SMILES: CC1(C)c2cc(N(c3ccc(-c4ccccc4)cc3)c3ccc(C4CC5CCC4C5)cc3)ccc2-c2cccc(-c3ccccc3)c2C1(C)C ; CC1(C)c2cc(N(c3ccc(C4CCCCC4)cc3)c3cccc(-c4ccccc4)c3)ccc2-c2cccc(-c3ccccc3)c2C1(C)C ; CC1(C)c2cc(N(c3ccc(C4CC5CCC4C5)cc3)c3cccc(-c4ccccc4)c3)ccc2-c2cccc(-c3ccccc3)c2C1(C)C]

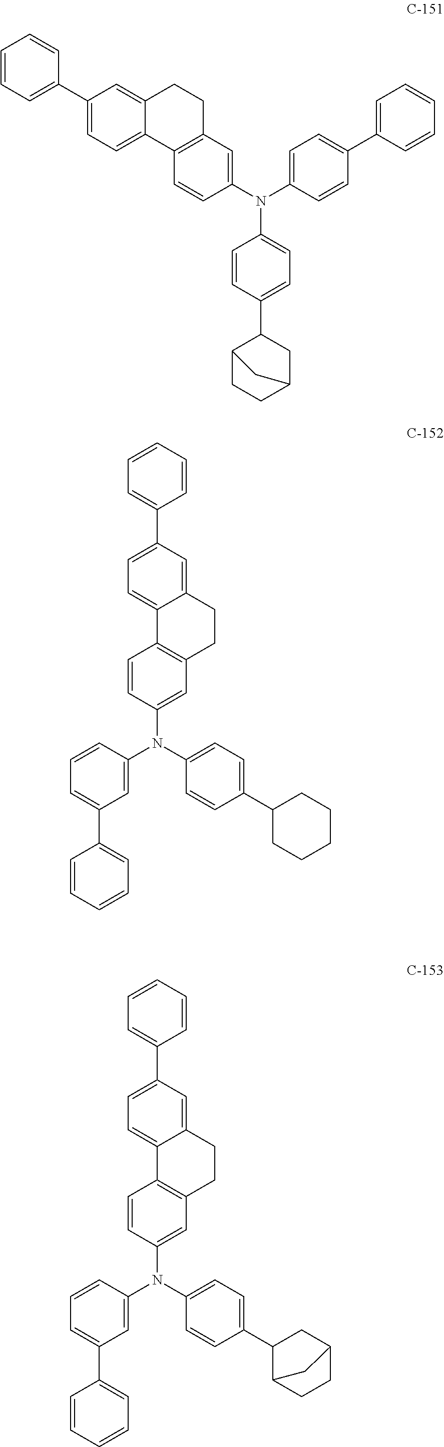 [SMILES: c1ccc(-c2ccc(N(c3ccc(C4CC5CCC4C5)cc3)c3ccc4c(c3)CCc3cc(-c5ccccc5)ccc3-4)cc2)cc1 ; c1ccc(-c2cccc(N(c3ccc(C4CCCCC4)cc3)c3ccc4c(c3)CCc3cc(-c5ccccc5)ccc3-4)c2)cc1 ; c1ccc(-c2cccc(N(c3ccc(C4CC5CCC4C5)cc3)c3ccc4c(c3)CCc3cc(-c5ccccc5)ccc3-4)c2)cc1]

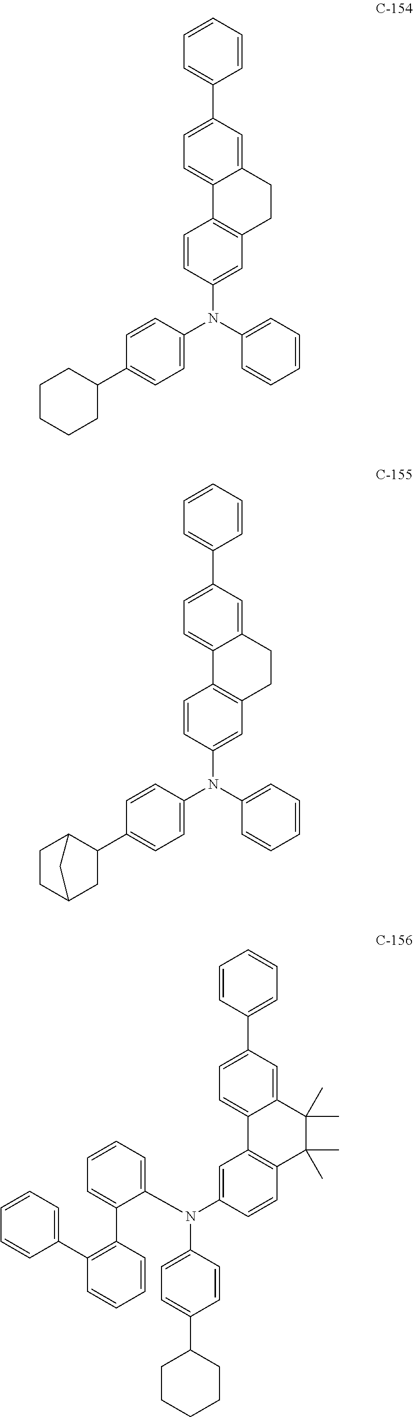 [SMILES: c1ccc(-c2ccc3c(c2)CCc2cc(N(c4ccccc4)c4ccc(C5CCCCC5)cc4)ccc2-3)cc1 ; c1ccc(-c2ccc3c(c2)CCc2cc(N(c4ccccc4)c4ccc(C5CC6CCC5C6)cc4)ccc2-3)cc1 ; CC1(C)c2ccc(N(c3ccc(C4CCCCC4)cc3)c3ccccc3-c3ccccc3-c3ccccc3)cc2-c2ccc(-c3ccccc3)cc2C1(C)C]

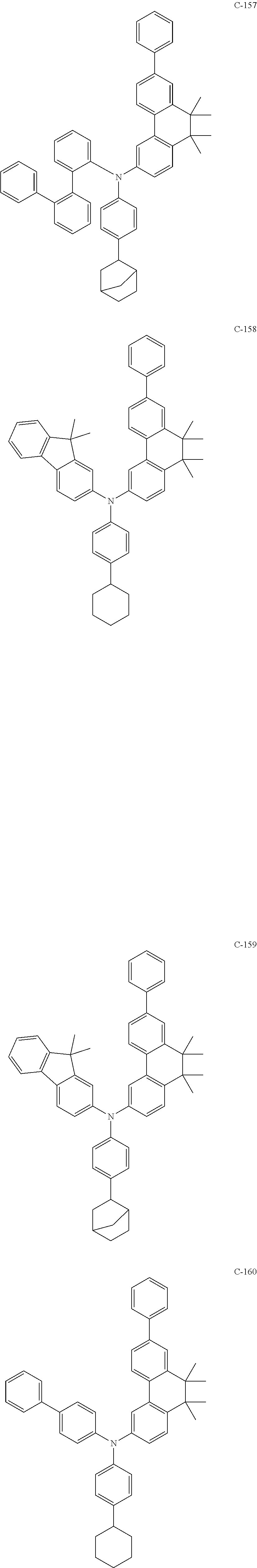 [SMILES: CC1(C)c2ccc(N(c3ccc(C4CC5CCC4C5)cc3)c3ccccc3-c3ccccc3-c3ccccc3)cc2-c2ccc(-c3ccccc3)cc2C1(C)C ; CC1(C)c2ccccc2-c2ccc(N(c3ccc(C4CCCCC4)cc3)c3ccc4c(c3)-c3ccc(-c5ccccc5)cc3C(C)(C)C4(C)C)cc21 ; CC1(C)c2ccccc2-c2ccc(N(c3ccc(C4CC5CCC4C5)cc3)c3ccc4c(c3)-c3ccc(-c5ccccc5)cc3C(C)(C)C4(C)C)cc21 ; CC1(C)c2ccc(N(c3ccc(-c4ccccc4)cc3)c3ccc(C4CCCCC4)cc3)cc2-c2ccc(-c3ccccc3)cc2C1(C)C]

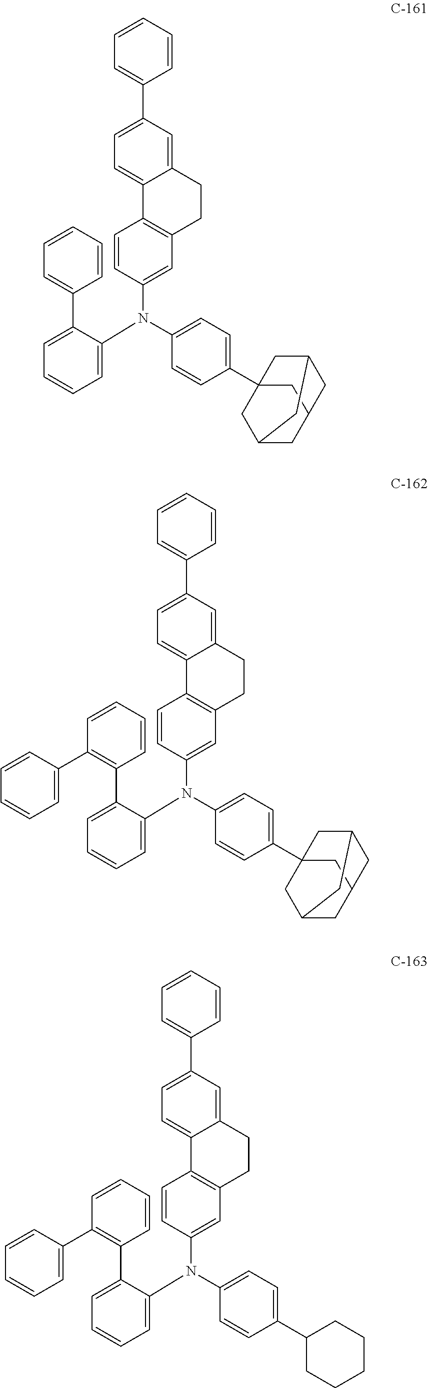 [SMILES: c1ccc(-c2ccc3c(c2)CCc2cc(N(c4ccc(C56CC7CC(CC(C7)C5)C6)cc4)c4ccccc4-c4ccccc4)ccc2-3)cc1 ; c1ccc(-c2ccc3c(c2)CCc2cc(N(c4ccc(C56CC7CC(CC(C7)C5)C6)cc4)c4ccccc4-c4ccccc4-c4ccccc4)ccc2-3)cc1 ; c1ccc(-c2ccc3c(c2)CCc2cc(N(c4ccc(C5CCCCC5)cc4)c4ccccc4-c4ccccc4-c4ccccc4)ccc2-3)cc1]

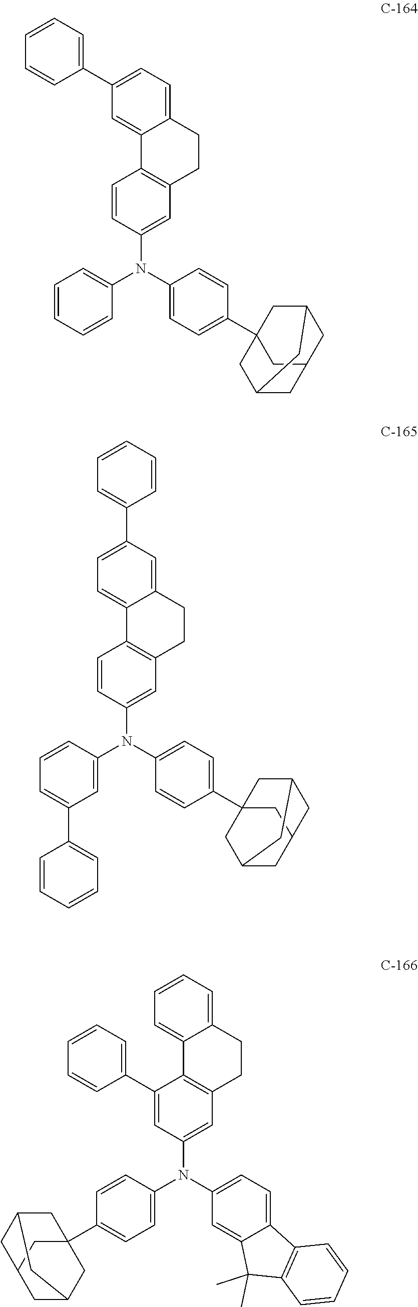 [SMILES: c1ccc(-c2ccc3c(c2)-c2ccc(N(c4ccccc4)c4ccc(C56CC7CC(CC(C7)C5)C6)cc4)cc2CC3)cc1 ; c1ccc(-c2cccc(N(c3ccc(C45CC6CC(CC(C6)C4)C5)cc3)c3ccc4c(c3)CCc3cc(-c5ccccc5)ccc3-4)c2)cc1 ; CC1(C)c2ccccc2-c2ccc(N(c3ccc(C45CC6CC(CC(C6)C4)C5)cc3)c3cc4c(c(-c5ccccc5)c3)-c3ccccc3CC4)cc21]

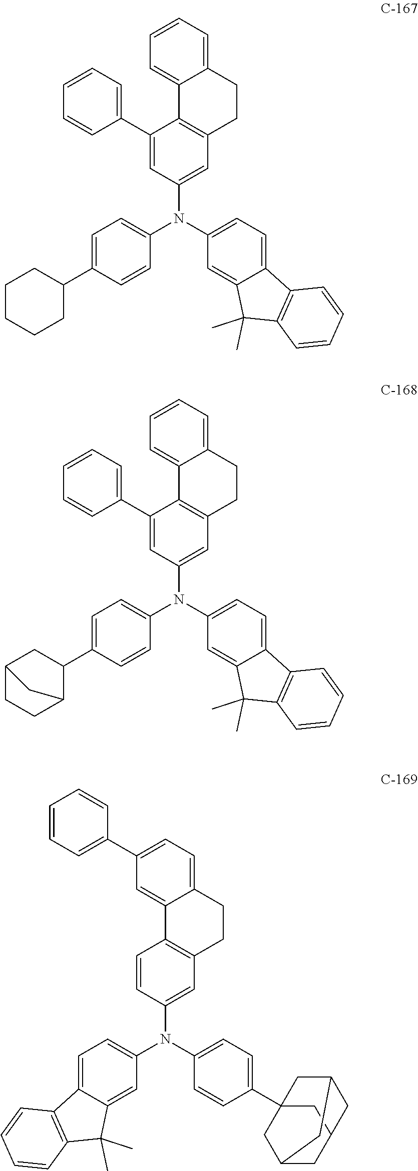 [SMILES: CC1(C)c2ccccc2-c2ccc(N(c3ccc(C4CCCCC4)cc3)c3cc4c(c(-c5ccccc5)c3)-c3ccccc3CC4)cc21 ; CC1(C)c2ccccc2-c2ccc(N(c3ccc(C4CC5CCC4C5)cc3)c3cc4c(c(-c5ccccc5)c3)-c3ccccc3CC4)cc21 ; CC1(C)c2ccccc2-c2ccc(N(c3ccc(C45CC6CC(CC(C6)C4)C5)cc3)c3ccc4c(c3)CCc3ccc(-c5ccccc5)cc3-4)cc21]

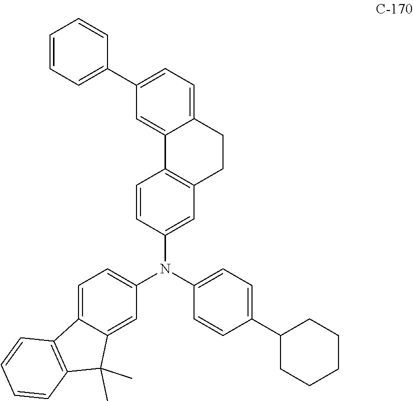 [SMILES: CC1(C)c2ccccc2-c2ccc(N(c3ccc(C4CCCCC4)cc3)c3ccc4c(c3)CCc3ccc(-c5ccccc5)cc3-4)cc21]

In order to form each layer of the organic electroluminescent device of the present disclosure, dry film-forming methods such as vacuum evaporation, sputtering, plasma, ion plating methods, etc. or wet film-forming methods such as spin coating, dip coating, flow coating methods, etc. may be used. When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

When forming a layer by the organic electroluminescent compound according to one embodiment, the layer may be formed by the methods listed above, and may often be formed by co-deposition or mixture-deposition. The co-deposition is a mixed deposition method in which two or more materials are put into respective individual crucible sources, and a current is applied to both cells simultaneously to evaporate the materials; and the mixed deposition is a method in which two or more materials are mixed in one crucible source before deposition, and then a current is applied to one cell to evaporate the materials.

According to one embodiment, the present disclosure can provide display devices comprising the organic electroluminescent compound represented by Formula 1. In addition, by using an organic electroluminescent device of the present disclosure, display devices such as smartphones, tablets, notebooks, PCs, TVs, or display devices for vehicles, or lighting devices such as outdoor or indoor lighting can be prepared.

Hereinafter, the preparation method of the compound according to the present disclosure will be explained with reference to the synthesis method of a representative compound or intermediate compound in order to understand the present disclosure in detail.

### [Example 1] Preparation of Compound C-51

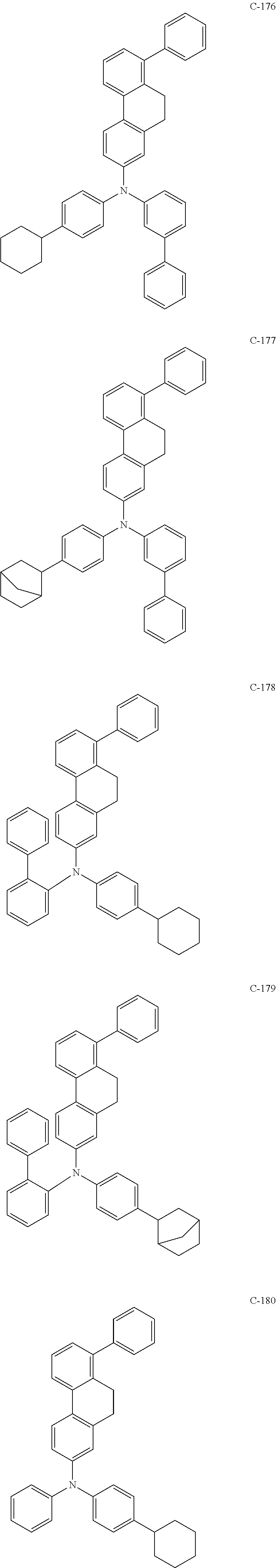 [SMILES: c1ccc(-c2cccc(N(c3ccc(C4CCCCC4)cc3)c3ccc4c(c3)CCc3c(-c5ccccc5)cccc3-4)c2)cc1 ; c1ccc(-c2cccc(N(c3ccc(C4CC5CCC4C5)cc3)c3ccc4c(c3)CCc3c(-c5ccccc5)cccc3-4)c2)cc1 ; c1ccc(-c2ccccc2N(c2ccc(C3CCCCC3)cc2)c2ccc3c(c2)CCc2c(-c4ccccc4)cccc2-3)cc1 ; c1ccc(-c2ccccc2N(c2ccc(C3CC4CCC3C4)cc2)c2ccc3c(c2)CCc2c(-c4ccccc4)cccc2-3)cc1 ; c1ccc(-c2cccc3c2CCc2cc(N(c4ccccc4)c4ccc(C5CCCCC5)cc4)ccc2-3)cc1]

### 1) Synthesis of Compound A

2-(4-Bromophenyl)bicyclo[2.2.1]heptane (18.3 g, 72.9 mmol), 4-(adamantan-1-yl)aniline (21.5 g, 94.7 mmol), Pd(OAc)₂ (0.818 g, 3.64 mmol), S-Phos (2.99 g, 7.29 mmol), NaOt-Bu (10.5 g, 109 mmol), and 300 mL of o-xylene were added to a flask and dissolved. The reaction mixture was then stirred under reflux at 180° C. for 4 hours. When the reaction was completed, the mixture was separated by column chromatography to obtain Compound A (18.0 g, yield: 62%).

### 2) Synthesis of Compound C-51

2-iodo-9,9,10,10-tetramethyl-phenanthrene (7.00 g, 19.3 mmol), Compound A (8.45 g, 21.3 mmol), Pd₂(dba)₃ (0.885 g, 0.966 mmol), P(t-Bu)₃ (0.821 g, 1.93 mmol), NaOt-Bu (3.71 g, 38.6 mmol), and 100 mL of toluene were added to a flask and dissolved. The reaction mixture was then stirred under reflux for 1 hour. When the reaction was completed, the solid produced by adding methanol to the reaction solution was filtered under reduced pressure and then separated by column chromatography to obtain Compound C-51 (3.7 g, yield: 30%).

| | MW | M.P. |
|---|---|---|
| C-51 | 631.93 | 178° C. |

### [Example 2] Synthesis of Compound C-52

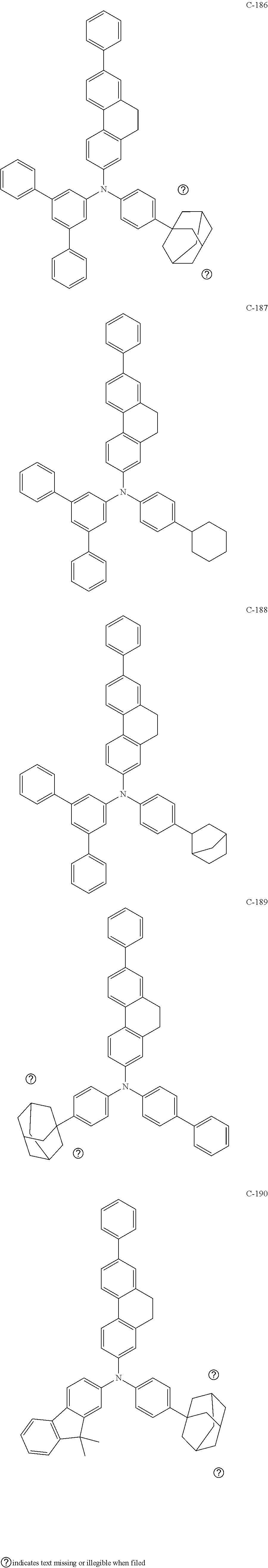 [SMILES: c1ccc(-c2cc(-c3ccccc3)cc(N(c3ccc(C45CC6CC(CC(C6)C4)C5)cc3)c3ccc4c(c3)CCc3cc(-c5ccccc5)ccc3-4)c2)cc1 ; c1ccc(-c2cc(-c3ccccc3)cc(N(c3ccc(C4CCCCC4)cc3)c3ccc4c(c3)CCc3cc(-c5ccccc5)ccc3-4)c2)cc1 ; c1ccc(-c2cc(-c3ccccc3)cc(N(c3ccc(C4CC5CCC4C5)cc3)c3ccc4c(c3)CCc3cc(-c5ccccc5)ccc3-4)c2)cc1 ; c1ccc(-c2ccc(N(c3ccc(C45CC6CC(CC(C6)C4)C5)cc3)c3ccc4c(c3)CCc3cc(-c5ccccc5)ccc3-4)cc2)cc1 ; CC1(C)c2ccccc2-c2ccc(N(c3ccc(C45CC6CC(CC(C6)C4)C5)cc3)c3ccc4c(c3)CCc3cc(-c5ccccc5)ccc3-4)cc21]

3-Bromo-9,9,10,10-tetramethyl-phenanthrene (15.0 g, 47.6 mmol), Compound A (19.9 g, 50.0 mmol), Pd₂(dba)₃ (2.18 g, 2.38 mmol), P(t-Bu)₃ (2.02 g, 4.76 mmol), NaOt-Bu (9.15 g, 95.2 mmol), and 250 mL of toluene were added to a flask and dissolved. The reaction mixture was then stirred under reflux for 1 hour. When the reaction was completed, the solid produced by adding methanol to the reaction solution was filtered under reduced pressure and then separated by column chromatography to obtain Compound C-52 (8.5 g, yield: 28.3%).

| | MW | M.P. |
|---|---|---|
| C-52 | 631.93 | 147.4° C. |

Hereinafter, the method for measuring the refractive index of the organic electroluminescent compound according to the present disclosure and the measurement results will be explained in order for the present disclosure to be understood in detail.

### [Examples 3 to 6] Measurement of Refractive Index of an Organic Electroluminescent Compound According to the Present Disclosure

To obtain the refractive index of the organic electroluminescent compounds according to the present disclosure, the material to be measured for refractive index was placed in a cell inside a vacuum vapor deposition apparatus, followed by evacuation until the degree of vacuum in the chamber reached 10⁻⁶ torr. Then the current was applied to the cell to evaporate the material, thereby preparing a sample with a thickness of 30 nm on a silicon wafer substrate.

The refractive index was measured using an ellipsometer. Specifically, a J.A. Woollam M-2000 from WIZOPTICS was used to measure the refractive index in the wavelength range of 350 nm to 800 nm under an incident angle of 60°, and the refractive index at 620 nm was used as the reference.

The results of measuring the refractive index of the representative compounds of the present disclosure using the measuring method above are shown in Table 1 below.

TABLE 1

| | Compound | Refractive Index |
|---|---|---|
| Example 3 | C-51 | 1.67 |
| Example 4 | C-52 | 1.64 |
| Example 5 | C-398 | 1.64 |
| Example 6 | C-284 | 1.71 |

From Table 1 above, it can be seen that the refractive index of the organic electroluminescent compound according to the present disclosure is 1.71 or less. Accordingly, it can be expected that when the organic electroluminescent compound of Formula 1 according to the present disclosure is used in an organic electroluminescent device, the light reflection loss will be reduced, thereby improving the current efficiency of the device.

Hereinafter, the preparation method of an organic electroluminescent device comprising the organic electroluminescent compound according to the present disclosure and the device properties thereof will be explained in order for the present disclosure to be understood in detail.

### [Device Examples 1 to 3] Preparation of OLEDs Comprising an Organic Electroluminescent Compound According to the Present Disclosure

OLEDs according to the present disclosure were prepared. First, a transparent electrode indium tin oxide (ITO) thin film (10 Ω/sq) on a glass substrate for an OLED (GEOMATEC CO., LTD.) was subjected to an ultrasonic washing with acetone and isopropyl alcohol, sequentially, and was thereafter stored in isopropyl alcohol and then used.

Thereafter, the ITO substrate was mounted on a substrate holder of a vacuum vapor deposition apparatus. Compound HI-1 was then introduced into a cell of the vacuum vapor deposition apparatus, and Compound HT-1 was introduced into another cell. The two materials were evaporated at different rates, and Compound HI-1 was deposited in a doping amount of 5 wt % based on the total amount of compounds HI-1 and HT-1 to form a hole injection layer having a thickness of 10 nm. Next, Compound HT-1 was deposited as a first hole transport layer having a thickness of 90 nm on the hole injection layer. The compound described in Table 2 below was then introduced as a second hole transport layer compound into another cell of the vacuum vapor deposition apparatus and was evaporated by applying current to the cell, thereby forming a second hole transport layer having a thickness of 60 nm on the first hole transport layer. Compound HT-2 was then introduced as a third hole transport layer compound into another cell of the vacuum vapor deposition apparatus and was evaporated by applying current to the cell, thereby forming a third hole transport layer having a thickness of 7.5 nm on the second hole transport layer. After forming the hole injection layer and the hole transport layers, a light-emitting layer was formed thereon as follows: Compound H-1 and Compound H-2 were introduced into two cells of the vacuum vapor deposition apparatus as hosts, and Compound D-39 was introduced into another cell as a dopant. The two host materials were evaporated at a rate of 1:1, and the dopant material was evaporated at a different rate, simultaneously, and was deposited in a doping amount of 2 wt % based on the total amount of the hosts and dopant to form a light-emitting layer having a thickness of 36 nm on the third hole transport layer. Compound B-2 was then deposited as an electron buffer material with a thickness of 5 nm on the light-emitting layer. Compounds ET-2 and EI-1 were then deposited as electron transport materials at a weight ratio of 2:1 to form an electron transport layer having a thickness of 25 nm. After depositing Compound EI-1 as an electron injection layer having a thickness of 2 nm on the electron transport layer, an Al cathode having a thickness of 80 nm was deposited on the electron injection layer by another vacuum vapor deposition apparatus. Thus, an OLED was produced. Each of the compounds used for all of the materials were purified by vacuum sublimation at 10⁻⁶ Torr.

### [Comparative Examples 1 and 2] Preparation of OLEDs Comprising Conventional Compounds as a Second Hole Transport Layer Material

OLEDs were manufactured in the same manner as in Device Example 1, except that the compounds shown in Table 2 below were used as the second hole transport layer materials.

The driving voltage, the current efficiency, and CIE color coordinates at a luminance of 1,000 nits of the organic electroluminescent devices of Examples 1 to 3 and Comparative Examples 1 and 2 produced as described above were measured, and the results thereof are shown in Table 2 below.

TABLE 2

| | | Driving | Current | CIE color |
|---|---|---|---|---|
| | Second hole | Voltage | Efficiency | coordinates |
| | transport layer | (V) | (cd/A) | (x, y) |
| Device Example 1 | C-51 | 2.9 | 31.2 | 0.660, 0.339 |
| Device Example 2 | C-52 | 2.8 | 30.9 | 0.661, 0.338 |
| Device Example 3 | C-398 | 2.9 | 32.2 | 0.662, 0.337 |
| Device | HT-3 | 2.9 | 28.8 | 0.660, 0.339 |
| Comparative | | | | |
| Example 1 | | | | |
| Device | HT-4 | 3.3 | 16.7 | 0.653, 0.345 |
| Comparative | | | | |
| Example 2 | | | | |

From Table 2 above, it can be seen that the organic electroluminescent device comprising the organic electroluminescent compounds according to the present disclosure as hole transport materials exhibits a lower driving voltage and/or higher efficiency characteristics compared to the organic electroluminescent device comprising conventional compounds as hole transport materials.

The compounds used in the Device Examples and Comparative Examples above are specifically shown in Table 3 below.

TABLE 3

| | |
|---|---|
| Hole Injection Layer/ Hole Transport Layer | 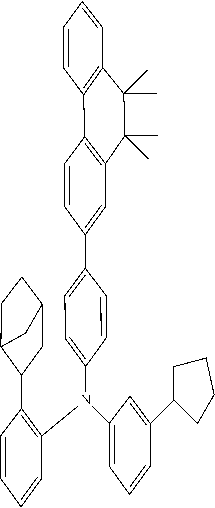 [SMILES: CC1(C)c2ccccc2-c2ccc(-c3ccc(N(c4cccc(C5CCCC5)c4)c4ccccc4C4CC5CCC4C5)cc3)cc2C1(C)C] |
| | C-266 |
| | 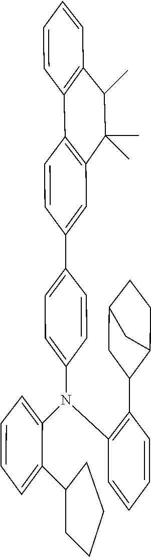 [SMILES: CC1c2ccccc2-c2ccc(-c3ccc(N(c4ccccc4C4CCCC4)c4ccccc4C4CC5CCC4C5)cc3)cc2C1(C)C] |
| | C-267 |
| | 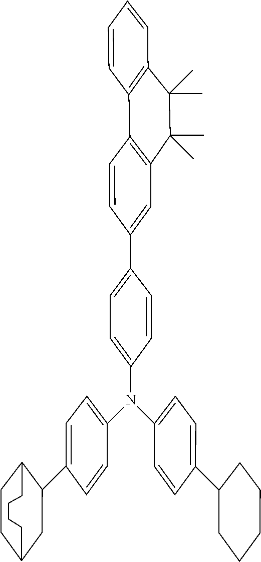 [SMILES: CC1(C)c2ccccc2-c2ccc(-c3ccc(N(c4ccc(C5CCCCC5)cc4)c4ccc(C5CC6CCCCC5CC6)cc4)cc3)cc2C1(C)C] |
| | C-268 |
| | 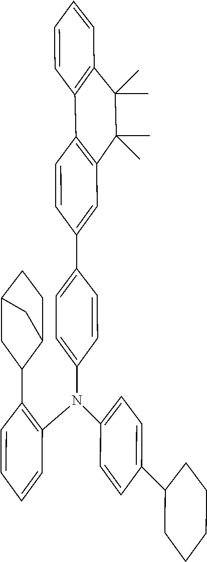 [SMILES: CC1(C)c2ccccc2-c2ccc(-c3ccc(N(c4ccc(C5CCCCC5)cc4)c4ccccc4C4CC5CCC4C5)cc3)cc2C1(C)C] |
| | C-269 |
| | 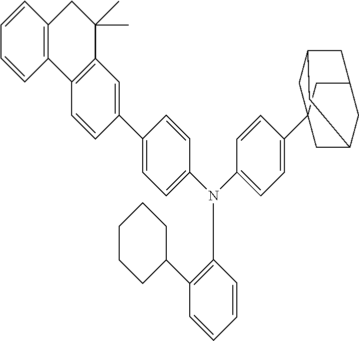 [SMILES: CC1(C)Cc2ccccc2-c2ccc(-c3ccc(N(c4ccc(C56CC7CC(CC(C7)C5)C6)cc4)c4ccccc4C4CCCCC4)cc3)cc21] |
| | C-270 |
| | 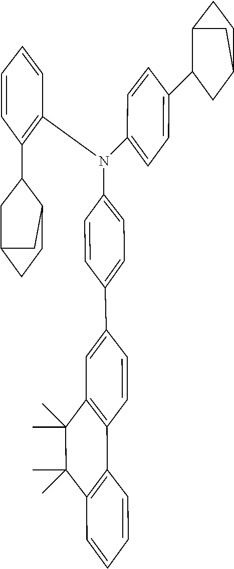 [SMILES: CC1(C)c2ccccc2-c2ccc(-c3ccc(N(c4ccc(C5CC6CCC5C6)cc4)c4ccccc4C4CC5CCC4C5)cc3)cc2C1(C)C] |
| | C-271 |
| | 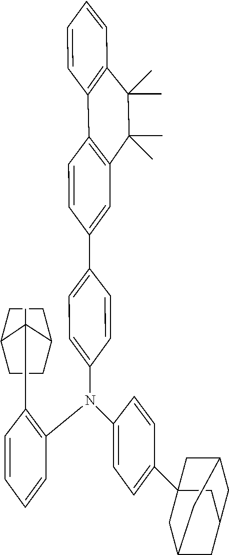 [SMILES: CC1(c2ccccc2N(c2ccc(-c3ccc4c(c3)C(C)(C)C(C)(C)c3ccccc3-4)cc2)c2ccc(C34CC5CC(CC(C5)C3)C4)cc2)C2CCC1CC2] |
| | C-272 |
| | 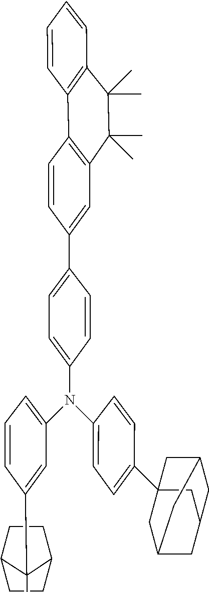 [SMILES: CC1(c2cccc(N(c3ccc(-c4ccc5c(c4)C(C)(C)C(C)(C)c4ccccc4-5)cc3)c3ccc(C45CC6CC(CC(C6)C4)C5)cc3)c2)C2CCC1CC2] |
| | C-273 |
| | 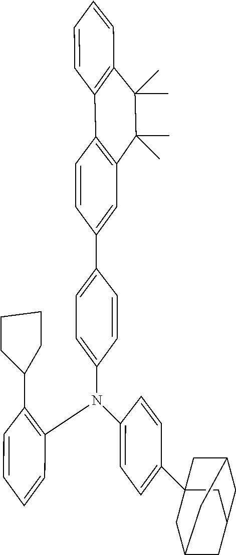 [SMILES: CC1(C)c2ccccc2-c2ccc(-c3ccc(N(c4ccc(C56CC7CC(CC(C7)C5)C6)cc4)c4ccccc4C4CCCC4)cc3)cc2C1(C)C] |
| | C-274 |
| | 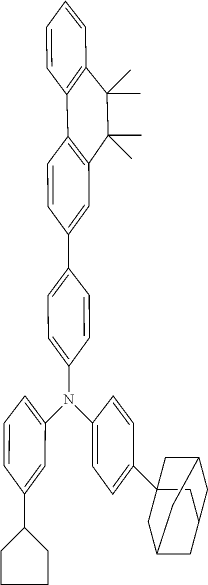 [SMILES: CC1(C)c2ccccc2-c2ccc(-c3ccc(N(c4ccc(C56CC7CC(CC(C7)C5)C6)cc4)c4cccc(C5CCCC5)c4)cc3)cc2C1(C)C] |
| | C-275 |
| | 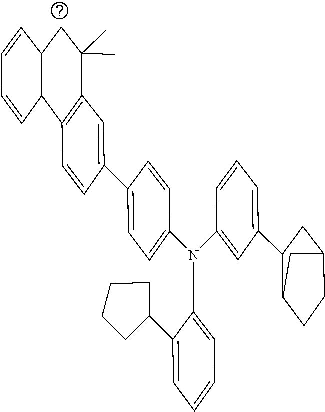 [SMILES: CC1(C)CC2C=CC=CC2c2ccc(-c3ccc(N(c4cccc(C5CC6CCC5C6)c4)c4ccccc4C4CCCC4)cc3)cc21] |
| | C-276 |
| | 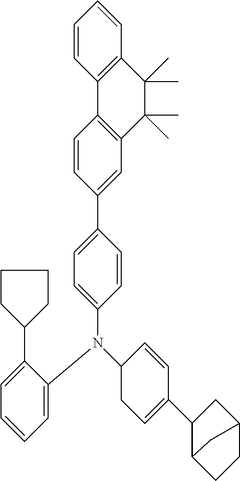 [SMILES: CC1(C)c2ccccc2-c2ccc(-c3ccc(N(c4ccccc4C4CCCC4)C4C=CC(C5CC6CCC5C6)=CC4)cc3)cc2C1(C)C] |
| | C-277 |
| | 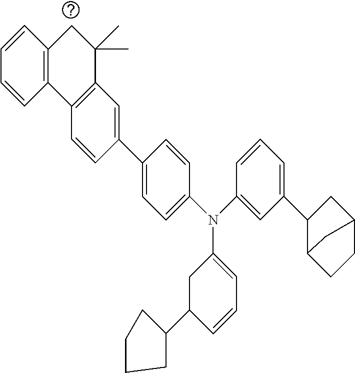 [SMILES: CC1(C)Cc2ccccc2-c2ccc(-c3ccc(N(C4=CC=CC(C5CCCC5)C4)c4cccc(C5CC6CCC5C6)c4)cc3)cc21] |
| | C-278 |
| | 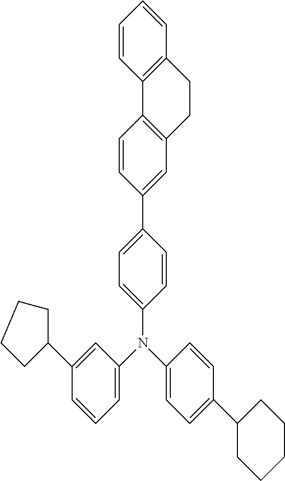 [SMILES: c1cc(C2CCCC2)cc(N(c2ccc(-c3ccc4c(c3)CCc3ccccc3-4)cc2)c2ccc(C3CCCCC3)cc2)c1] |
| | C-279 |
| | 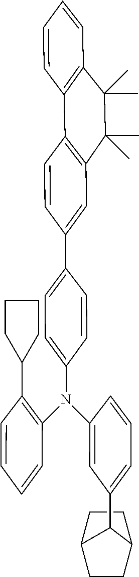 [SMILES: CC1(C)c2ccccc2-c2ccc(-c3ccc(N(c4cccc(C5C6CCC5CC6)c4)c4ccccc4C4CCCC4)cc3)cc2C1(C)C] |
| | C-280 |
| | HI-1 |
| | HT-1 |
| | HT-2 |
| | 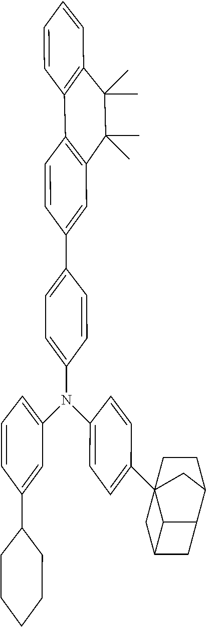 |
| | C-281 |
| | 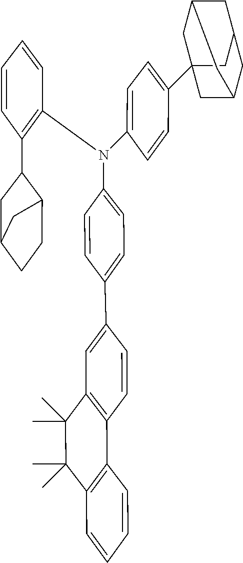 [SMILES: CC1(C)c2ccccc2-c2ccc(-c3ccc(N(c4ccc(C56CC7CC(CC(C7)C5)C6)cc4)c4ccccc4C4CC5CCC4C5)cc3)cc2C1(C)C] |
| | C-282 |
| | 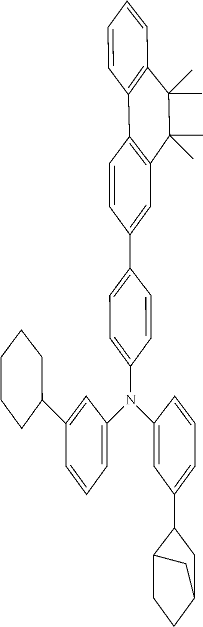 [SMILES: CC1(C)c2ccccc2-c2ccc(-c3ccc(N(c4cccc(C5CCCCC5)c4)c4cccc(C5CC6CCC5C6)c4)cc3)cc2C1(C)C] |
| | C-283 |
| | 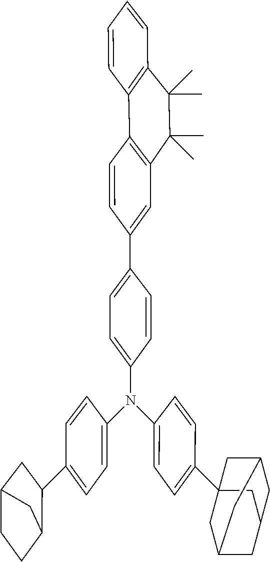 [SMILES: CC1(C)c2ccccc2-c2ccc(-c3ccc(N(c4ccc(C5CC6CCC5C6)cc4)c4ccc(C56CC7CC(CC(C7)C5)C6)cc4)cc3)cc2C1(C)C] |
| | C-284 |
| | 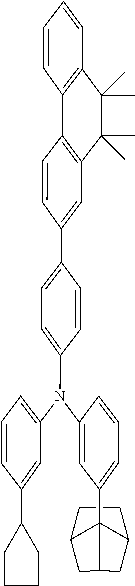 [SMILES: CC1(C)c2ccccc2-c2ccc(-c3ccc(N(c4cccc(C5CCCC5)c4)c4cccc(C56C7CCC5CC6C7)c4)cc3)cc2C1(C)C] |
| | C-285 |
| | 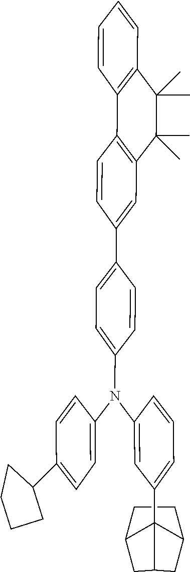 [SMILES: CC1(C)c2ccccc2-c2ccc(-c3ccc(N(c4ccc(C5CCCC5)cc4)c4cccc(C56C7CCC5CC6C7)c4)cc3)cc2C1(C)C] |
| | C-286 |
| | 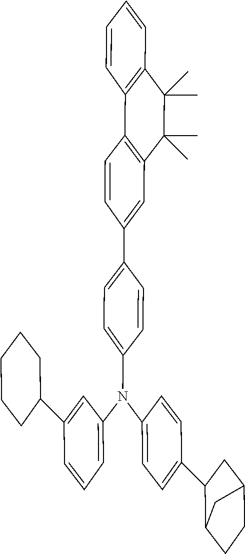 [SMILES: CC1(C)c2ccccc2-c2ccc(-c3ccc(N(c4ccc(C5CC6CCC5C6)cc4)c4cccc(C5CCCCC5)c4)cc3)cc2C1(C)C] |
| | C-287 |
| | 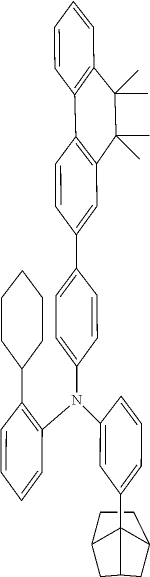 [SMILES: CC1(C)c2ccccc2-c2ccc(-c3ccc(N(c4cccc(C56CC7CC5CCC6C7)c4)c4ccccc4C4CCCCC4)cc3)cc2C1(C)C] |
| | C-288 |
| | 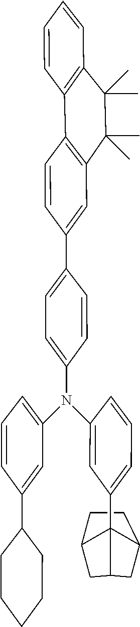 [SMILES: CC1(C)c2ccccc2-c2ccc(-c3ccc(N(c4cccc(C5CCCCC5)c4)c4cccc(C56CC7CC5CCC6C7)c4)cc3)cc2C1(C)C] |
| | C-289 |
| | 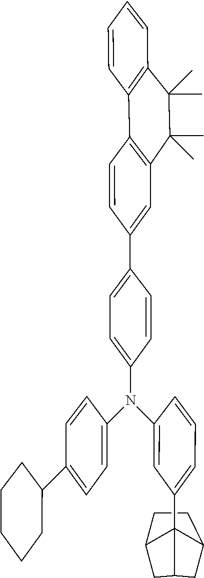 [SMILES: CC1(C)c2ccccc2-c2ccc(-c3ccc(N(c4ccc(C5CCCCC5)cc4)c4cccc(C56CC7CC5CCC6C7)c4)cc3)cc2C1(C)C] |
| | C-290 |
| | 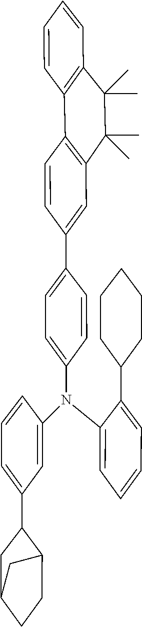 [SMILES: CC1(C)c2ccccc2-c2ccc(-c3ccc(N(c4cccc(C5CC6CCC5C6)c4)c4ccccc4C4CCCCC4)cc3)cc2C1(C)C] |
| | C-291 |
| | 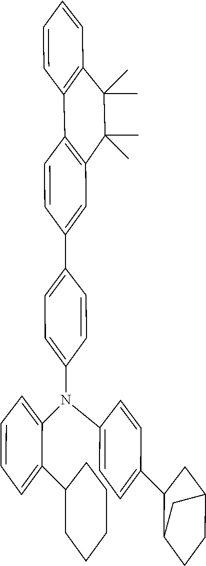 [SMILES: CC1(C)c2ccccc2-c2ccc(-c3ccc(N(c4ccc(C5CC6CCC5C6)cc4)c4ccccc4C4CCCCC4)cc3)cc2C1(C)C] |
| | C-292 |
| | 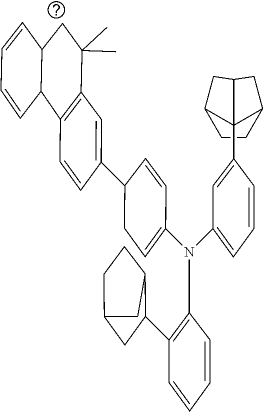 [SMILES: CC1(C)CC2C=CC=CC2c2ccc(C3C=CC(N(c4cccc(C56C7CCC5CC6C7)c4)c4ccccc4C4CC5CCC4C5)=CC3)cc21] |
| | C-293 |
| | 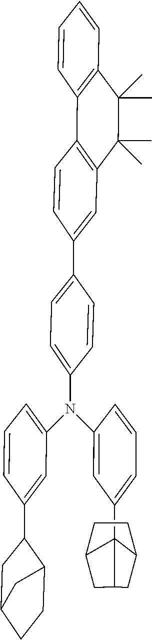 [SMILES: CC1(C)c2ccccc2-c2ccc(-c3ccc(N(c4cccc(C5CC6CCC5C6)c4)c4cccc(C56C7CCC5CC6C7)c4)cc3)cc2C1(C)C] |
| | C-294 |
| | 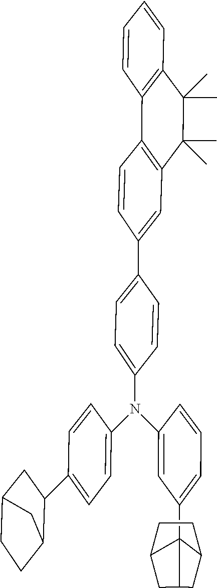 [SMILES: CC1(C)c2ccccc2-c2ccc(-c3ccc(N(c4ccc(C5CC6CCC5C6)cc4)c4cccc(C56C7CCC5CC6C7)c4)cc3)cc2C1(C)C] |
| | C-295 |
| | C-51 |
| | C-52 |
| | C-398 |
| | 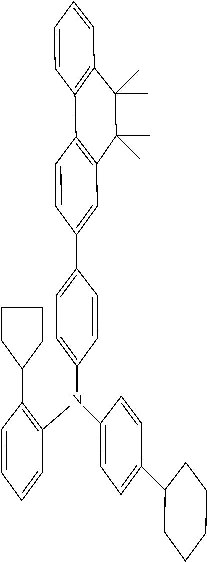 [SMILES: CC1(C)c2ccccc2-c2ccc(-c3ccc(N(c4ccc(C5CCCCC5)cc4)c4ccccc4C4CCCC4)cc3)cc2C1(C)C] |
| | C-296 |
| | 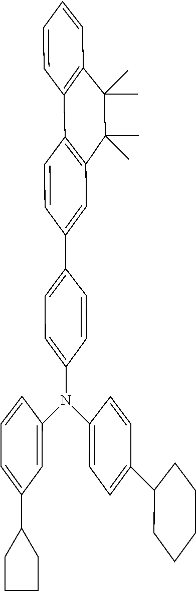 [SMILES: CC1(C)c2ccccc2-c2ccc(-c3ccc(N(c4ccc(C5CCCCC5)cc4)c4cccc(C5CCCC5)c4)cc3)cc2C1(C)C] |
| | C-297 |
| | 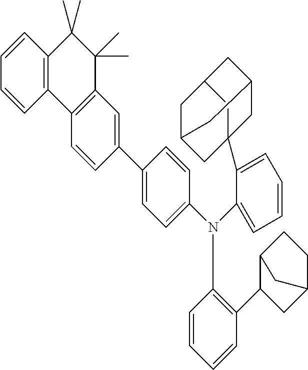 [SMILES: CC1(C)c2ccccc2-c2ccc(-c3ccc(N(c4ccccc4C4CC5CCC4C5)c4ccccc4C45CC6CC(CC(C6)C4)C5)cc3)cc2C1(C)C] |
| | C-298 |
| | 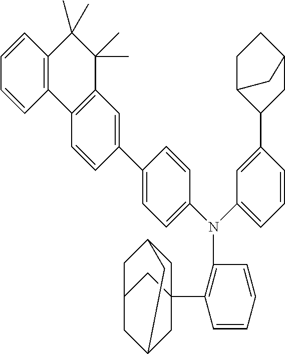 [SMILES: CC1(C)c2ccccc2-c2ccc(-c3ccc(N(c4cccc(C5CC6CCC5C6)c4)c4ccccc4C45CC6CC(CC(C6)C4)C5)cc3)cc2C1(C)C] |
| | C-299 |
| | 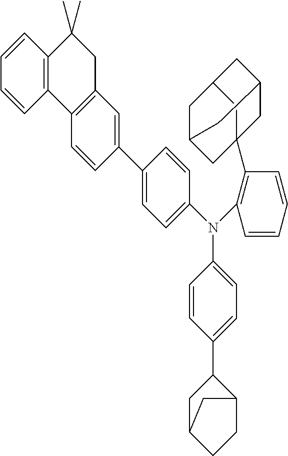 [SMILES: CC1(C)Cc2cc(-c3ccc(N(c4ccc(C5CC6CCC5C6)cc4)c4ccccc4C45CC6CC(CC(C6)C4)C5)cc3)ccc2-c2ccccc21] |
| | C-300 |
| | 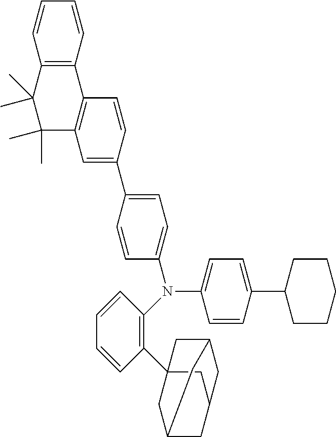 [SMILES: CC1(C)c2ccccc2-c2ccc(-c3ccc(N(c4ccc(C5CCCCC5)cc4)c4ccccc4C45CC6CC(CC(C6)C4)C5)cc3)cc2C1(C)C] |
| | C-301 |
| | 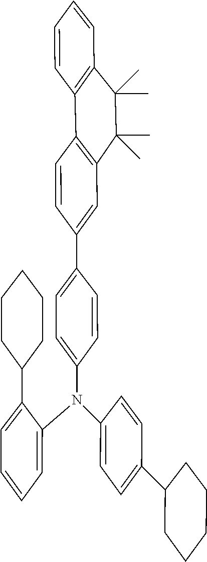 [SMILES: CC1(C)c2ccccc2-c2ccc(-c3ccc(N(c4ccc(C5CCCCC5)cc4)c4ccccc4C4CCCCC4)cc3)cc2C1(C)C] |
| | C-302 |
| | 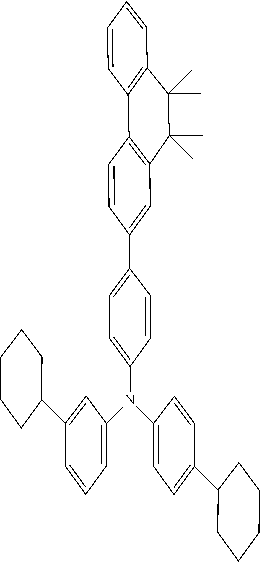 [SMILES: CC1(C)c2ccccc2-c2ccc(-c3ccc(N(c4ccc(C5CCCCC5)cc4)c4cccc(C5CCCCC5)c4)cc3)cc2C1(C)C] |
| | C-303 |
| | 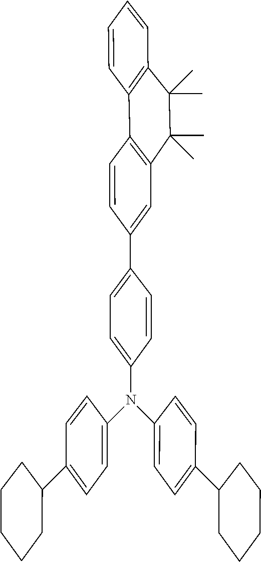 [SMILES: CC1(C)c2ccccc2-c2ccc(-c3ccc(N(c4ccc(C5CCCCC5)cc4)c4ccc(C5CCCCC5)cc4)cc3)cc2C1(C)C] |
| | C-304 |
| | 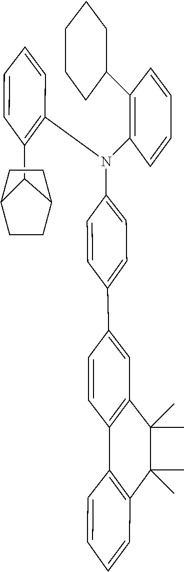 [SMILES: CC1(C)c2ccccc2-c2ccc(-c3ccc(N(c4ccccc4C4CCCCC4)c4ccccc4C4C5CCC4CC5)cc3)cc2C1(C)C] |
| | C-305 |
| | HT-3 |
| | HT-4 |
| Light- Emitting Layer | 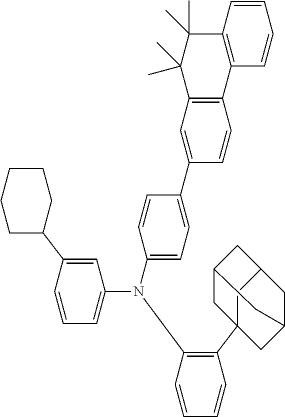 [SMILES: CC1(C)c2ccccc2-c2ccc(-c3ccc(N(c4cccc(C5CCCCC5)c4)c4ccccc4C45CC6CC(CC(C6)C4)C5)cc3)cc2C1(C)C] |
| | C-306 |
| | 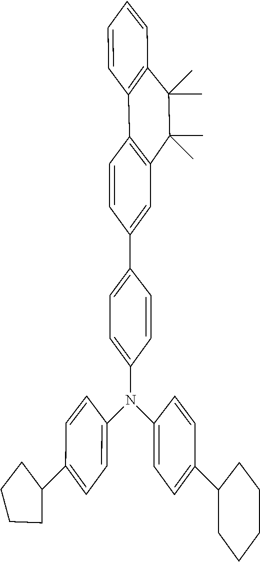 [SMILES: CC1(C)c2ccccc2-c2ccc(-c3ccc(N(c4ccc(C5CCCCC5)cc4)c4ccc(C5CCCC5)cc4)cc3)cc2C1(C)C] |
| | C-307 |
| | 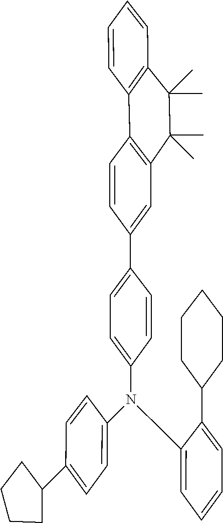 [SMILES: CC1(C)c2ccccc2-c2ccc(-c3ccc(N(c4ccc(C5CCCC5)cc4)c4ccccc4C4CCCCC4)cc3)cc2C1(C)C] |
| | C-308 |
| | 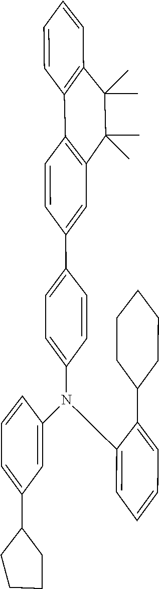 [SMILES: CC1(C)c2ccccc2-c2ccc(-c3ccc(N(c4cccc(C5CCCC5)c4)c4ccccc4C4CCCCC4)cc3)cc2C1(C)C] |
| | C-309 |
| | 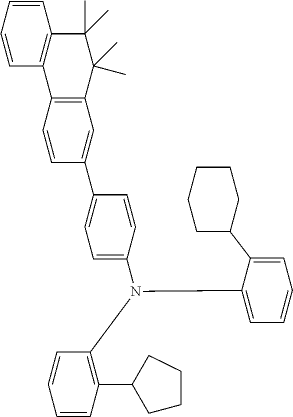 [SMILES: CC1(C)c2ccccc2-c2ccc(-c3ccc(N(c4ccccc4C4CCCCC4)c4ccccc4C4CCCC4)cc3)cc2C1(C)C] |
| | C-310 |
| | H-1 |
| | 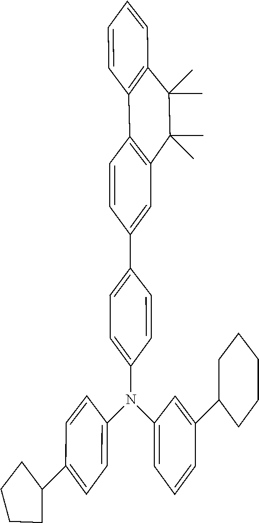 [SMILES: CC1(C)c2ccccc2-c2ccc(-c3ccc(N(c4ccc(C5CCCC5)cc4)c4cccc(C5CCCCC5)c4)cc3)cc2C1(C)C] |
| | C-311 |
| | 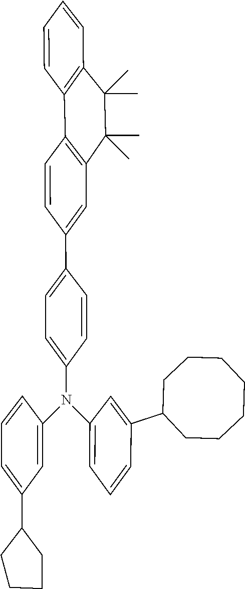 [SMILES: CC1(C)c2ccccc2-c2ccc(-c3ccc(N(c4cccc(C5CCCCCCC5)c4)c4cccc(C5CCCC5)c4)cc3)cc2C1(C)C] |
| | C-312 |
| | 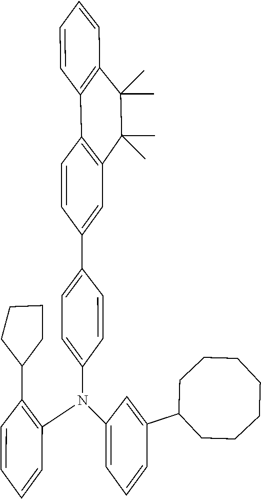 [SMILES: CC1(C)c2ccccc2-c2ccc(-c3ccc(N(c4cccc(C5CCCCCCC5)c4)c4ccccc4C4CCCC4)cc3)cc2C1(C)C] |
| | C-313 |
| | 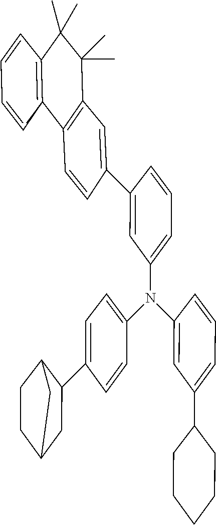 |
| | C-314 |
| | 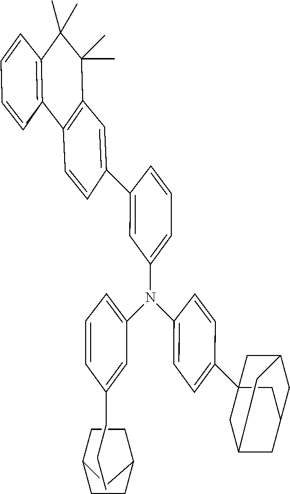 |
| | C-315 |
| | 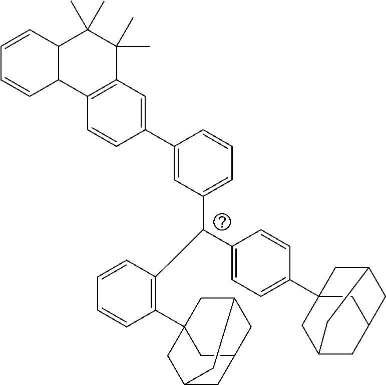 [SMILES: CC1(C)c2cc(-c3cccc(C(c4ccc(C56CC7CC(CC(C7)C5)C6)cc4)c4ccccc4C45CC6CC(CC(C6)C4)C5)c3)ccc2C2C=CC=CC2C1(C)C] |
| | C-316 |
| | 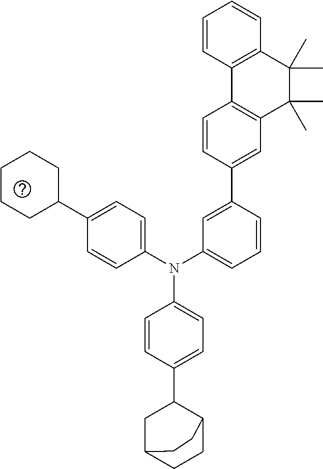 [SMILES: CC1(C)c2ccccc2-c2ccc(-c3cccc(N(c4ccc(C5CCCCC5)cc4)c4ccc(C5CC6CCC5CC6)cc4)c3)cc2C1(C)C] |
| | C-317 |
| | 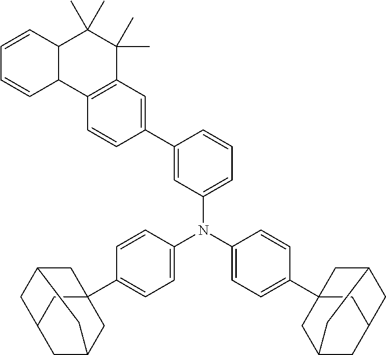 [SMILES: CC1(C)c2cc(-c3cccc(N(c4ccc(C56CC7CC(CC(C7)C5)C6)cc4)c4ccc(C56CC7CC(CC(C7)C5)C6)cc4)c3)ccc2C2C=CC=CC2C1(C)C] |
| | C-318 |
| | 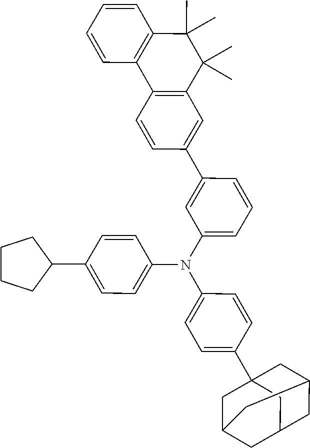 [SMILES: CC1(C)c2ccccc2-c2ccc(-c3cccc(N(c4ccc(C5CCCC5)cc4)c4ccc(C56CC7CC(CC(C7)C5)C6)cc4)c3)cc2C1(C)C] |
| | C-319 |
| | 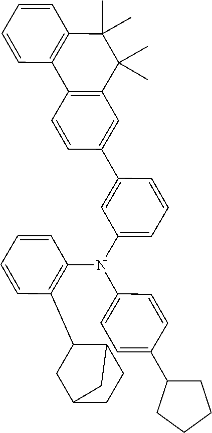 [SMILES: CC1(C)c2ccccc2-c2ccc(-c3cccc(N(c4ccc(C5CCCC5)cc4)c4ccccc4C4CC5CCC4C5)c3)cc2C1(C)C] |
| | C-320 |
| | H-2 |
| | D-39 |
| Electron Buffer Layer/ Electron Transport Layer/ Electron Injection Layer | 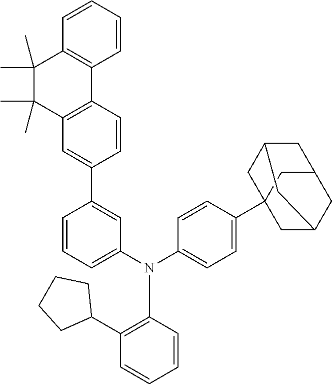 [SMILES: CC1(C)c2ccccc2-c2ccc(-c3cccc(N(c4ccc(C56CC7CC(CC(C7)C5)C6)cc4)c4ccccc4C4CCCC4)c3)cc2C1(C)C] |
| | C-321 |
| | 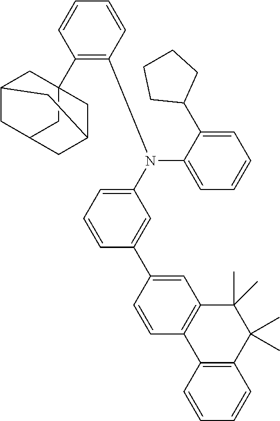 [SMILES: CC1(C)c2ccccc2-c2ccc(-c3cccc(N(c4ccccc4C4CCCC4)c4ccccc4C45CC6CC(CC(C6)C4)C5)c3)cc2C1(C)C] |
| | C-322 |
| | 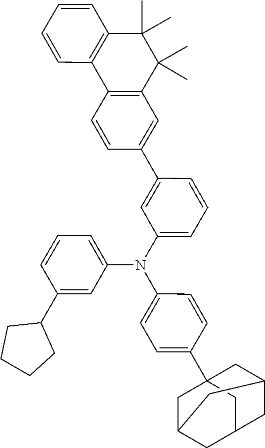 [SMILES: CC1(C)c2ccccc2-c2ccc(-c3cccc(N(c4ccc(C56CC7CC(CC(C7)C5)C6)cc4)c4cccc(C5CCCC5)c4)c3)cc2C1(C)C] |
| | C-323 |
| | 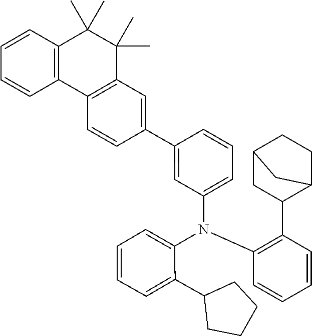 [SMILES: CC1(C)c2ccccc2-c2ccc(-c3cccc(N(c4ccccc4C4CCCC4)c4ccccc4C4CC5CCC4C5)c3)cc2C1(C)C] |
| | C-324 |
| | 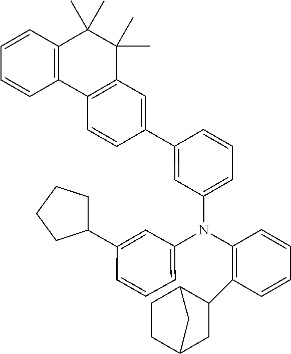 [SMILES: CC1(C)c2ccccc2-c2ccc(-c3cccc(N(c4cccc(C5CCCC5)c4)c4ccccc4C4CC5CCC4C5)c3)cc2C1(C)C] |
| | C-325 |
| | B-2 |
| | 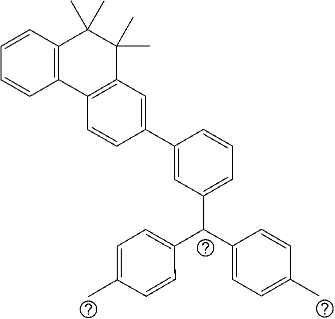 [SMILES: Cc1ccc(C(c2ccc(C)cc2)c2cccc(-c3ccc4c(c3)C(C)(C)C(C)(C)c3ccccc3-4)c2)cc1] |
| | C-326 |
| | 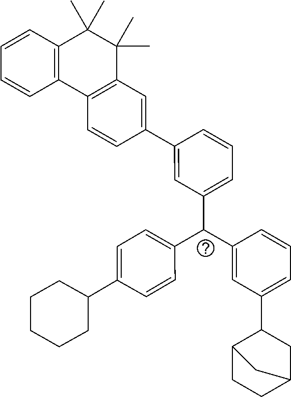 [SMILES: CC1(C)c2ccccc2-c2ccc(-c3cccc(C(c4ccc(C5CCCCC5)cc4)c4cccc(C5CC6CCC5C6)c4)c3)cc2C1(C)C] |
| | C-327 |
| | 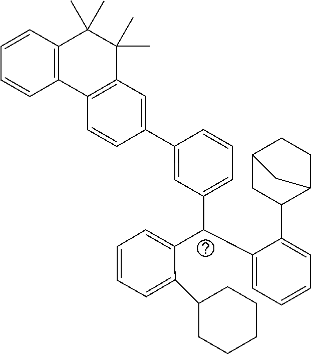 [SMILES: CC1(C)c2ccccc2-c2ccc(-c3cccc(C(c4ccccc4C4CCCCC4)c4ccccc4C4CC5CCC4C5)c3)cc2C1(C)C] |
| | C-328 |
| | 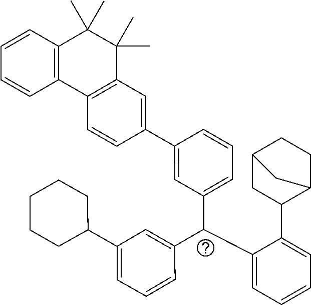 [SMILES: CC1(C)c2ccccc2-c2ccc(-c3cccc(C(c4cccc(C5CCCCC5)c4)c4ccccc4C4CC5CCC4C5)c3)cc2C1(C)C] |
| | C-329 |
| | 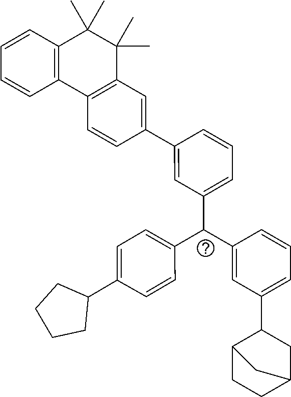 [SMILES: CC1(C)c2ccccc2-c2ccc(-c3cccc(C(c4ccc(C5CCCC5)cc4)c4cccc(C5CC6CCC5C6)c4)c3)cc2C1(C)C] |
| | C-330 |
| | 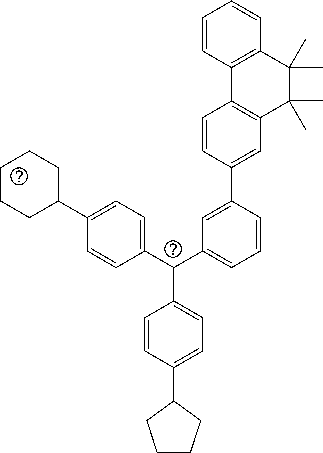 [SMILES: CC1(C)c2ccccc2-c2ccc(-c3cccc(C(c4ccc(C5CCCCC5)cc4)c4ccc(C5CCCC5)cc4)c3)cc2C1(C)C] |
| | C-331 |
| | 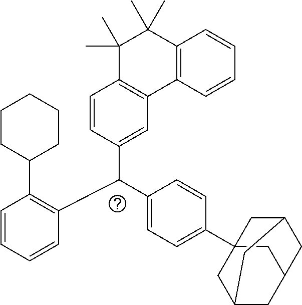 [SMILES: CC1(C)c2ccccc2-c2cc(C(c3ccc(C45CC6CC(CC(C6)C4)C5)cc3)c3ccccc3C3CCCCC3)ccc2C1(C)C] |
| | C-332 |
| | 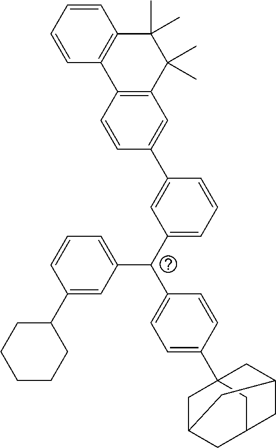 [SMILES: CC1(C)c2ccccc2-c2ccc(-c3cccc(C(c4ccc(C56CC7CC(CC(C7)C5)C6)cc4)c4cccc(C5CCCCC5)c4)c3)cc2C1(C)C] |
| | C-333 |
| | 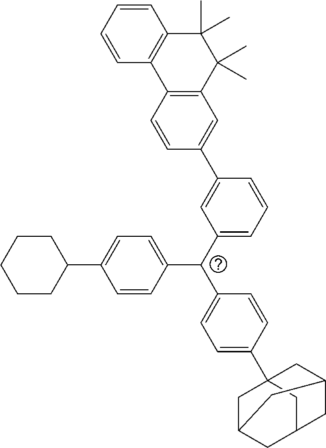 [SMILES: CC1(C)c2ccccc2-c2ccc(-c3cccc(C(c4ccc(C5CCCCC5)cc4)c4ccc(C56CC7CC(CC(C7)C5)C6)cc4)c3)cc2C1(C)C] |
| | C-334 |
| | 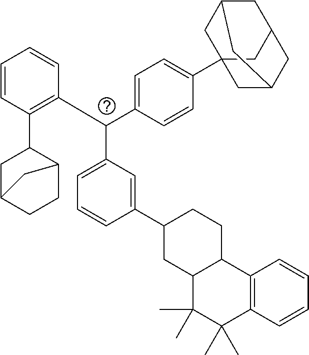 [SMILES: CC1(C)c2ccccc2C2CCC(c3cccc(C(c4ccc(C56CC7CC(CC(C7)C5)C6)cc4)c4ccccc4C4CC5CCC4C5)c3)CC2C1(C)C] |
| | C-335 |
| | ET-2 |
| | EI-1 |

1. An organic electroluminescent compound represented by the following Formula 1: 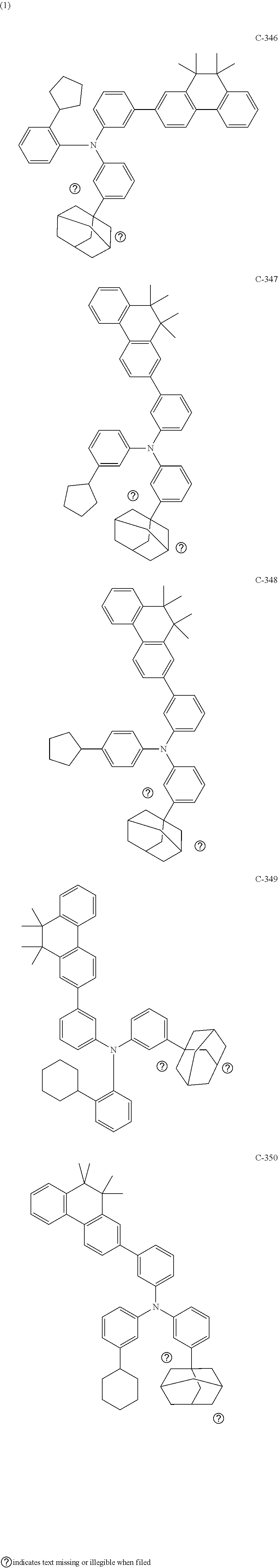
wherein,
ring A and ring B each independently represent a substituted or unsubstituted (C₆-C₃₀)arene, or a substituted or unsubstituted (3- to 30-membered)heteroarene;
R₁ to R₄, Rₐ, and R_{b} each independently represent hydrogen, deuterium, halogen, cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring of (C₃-C₃₀) aliphatic ring and (C₆-C₃₀) aromatic ring, a substituted or unsubstituted mono- or di(C₁-C₃₀)alkylamino, a substituted or unsubstituted mono- or di(C₆-C₃₀)arylamino, a substituted or unsubstituted (C₁-C₃₀)alkyl(C₆-C₃₀)arylamino, a substituted or unsubstituted mono- or di(3-to 30-membered)heteroarylamino, or a substituted or unsubstituted (C₆-C₃₀)aryl(3- to 30-membered)heteroarylamino; or may be linked to the adjacent substituents to form a ring(s);
provided that at least one of R₁ to R₄, Rₐ, and R_{b} is represented by the following Formula 1-1: 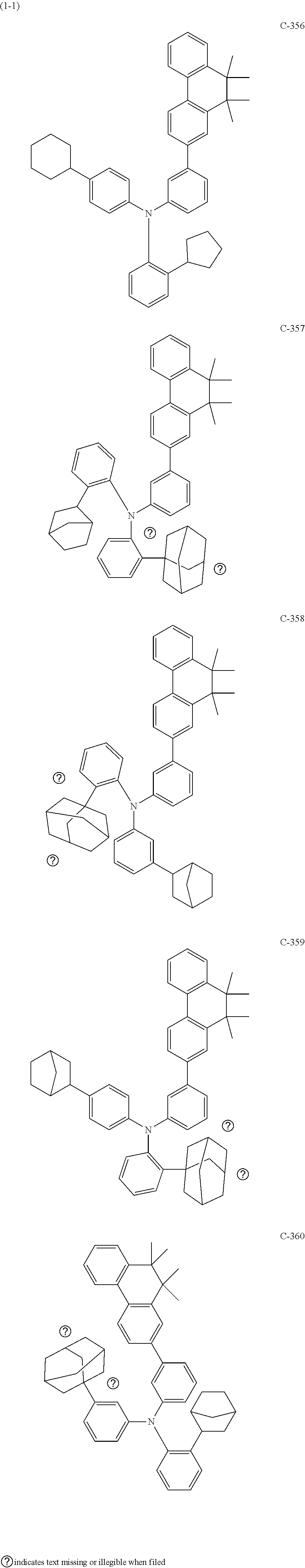
wherein,
L₁ to L₃ each independently represent a single bond, a substituted or unsubstituted (C₆-C₃₀)arylene, or a substituted or unsubstituted (3- to 30-membered)heteroarylene;
Ar₁ and Ar₂ each independently represent a substituted or unsubstituted (C₆-C₃₀)aryl, a substituted or unsubstituted (3- to 30-membered)heteroaryl, or a substituted or unsubstituted (C₃-C₃₀)cycloalkyl;
provided that at least one of Ar₁ and Ar₂ is a substituted or unsubstituted (C₃-C₃₀)cycloalkyl; and
n and m are integers of 1 to 8, and when n and m are integers greater than or equal to 2, each of Rₐ and each of R_{b} may be the same or different.

2. The organic electroluminescent compound according to claim 1, wherein the substituted arene, the substituted heteroarene, the substituted alkyl, the substituted aryl(ene), the substituted heteroaryl(ene), the substituted cycloalkyl, the substituted alkoxy, the substituted trialkylsilyl, the substituted dialkylarylsilyl, the substituted alkyldiarylsilyl, the substituted triarylsilyl, the substituted fused ring of aliphatic ring and aromatic ring, the substituted mono- or dialkylamino, the substituted mono- or diarylamino, the substituted alkylarylamino, the substituted mono- or diheteroarylamino, and the substituted arylheteroarylamino each independently is substituted with at least one selected from the group consisting of deuterium; halogen; cyano; carboxyl; nitro; hydroxy; (C₁-C₃₀)alkyl; halo(C₁-C₃₀)alkyl; (C₂-C₃₀)alkenyl; (C₂-C₃₀)alkynyl; (C₁-C₃₀)alkoxy; (C₁-C₃₀)alkylthio; (C₃-C₃₀)cycloalkyl; (C₃-C₃₀)cycloalkenyl; (3- to 7-membered)heterocycloalkyl; (C₆-C₃₀)aryloxy; (C₆-C₃₀)arylthio; (5- to 30-membered)heteroaryl substituted or unsubstituted with (C₆-C₃₀)aryl; (C₆-C₃₀)aryl substituted or unsubstituted with (5- to 30-membered)heteroaryl; tri(C₁-C₃₀)alkylsilyl; tri(C₆-C₃₀)arylsilyl; di(C₁-C₃₀)alkyl(C₆-C₃₀)arylsilyl; (C₁-C₃₀)alkyldi(C₆-C₃₀)arylsilyl; a fused ring of (C₃-C₃₀)aliphatic ring and (C₆-C₃₀)aromatic ring; amino; mono- or di(C₁-C₃₀)alkylamino; a substituted or unsubstituted mono- or di(C₆-C₃₀)arylamino; (C₁-C₃₀)alkyl(C₆-C₃₀)arylamino; mono- or di(3- to 30-membered)heteroarylamino; (C₁-C₃₀)alkyl(3- to 30-membered)heteroarylamino; (C₆-C₃₀)aryl(3- to 30-membered)heteroarylamino; (C₁-C₃₀)alkylcarbonyl; (C₁-C₃₀)alkoxycarbonyl; (C₆-C₃₀)arylcarbonyl; (C₆-C₃₀)arylphosphinyl, di(C₆-C₃₀)arylboronyl; di(C₁-C₃₀)alkylboronyl; (C₁-C₃₀)alkyl(C₆-C₃₀)arylboronyl; (C₆-C₃₀)ar(C₁-C₃₀)alkyl; (C₁-C₃₀)alkyl(C₆-C₃₀)aryl, and a combination thereof.

3. The organic electroluminescent compound according to claim 1, wherein Formula 1 is represented by any one of the following Formulas 2-1 to 2-11: 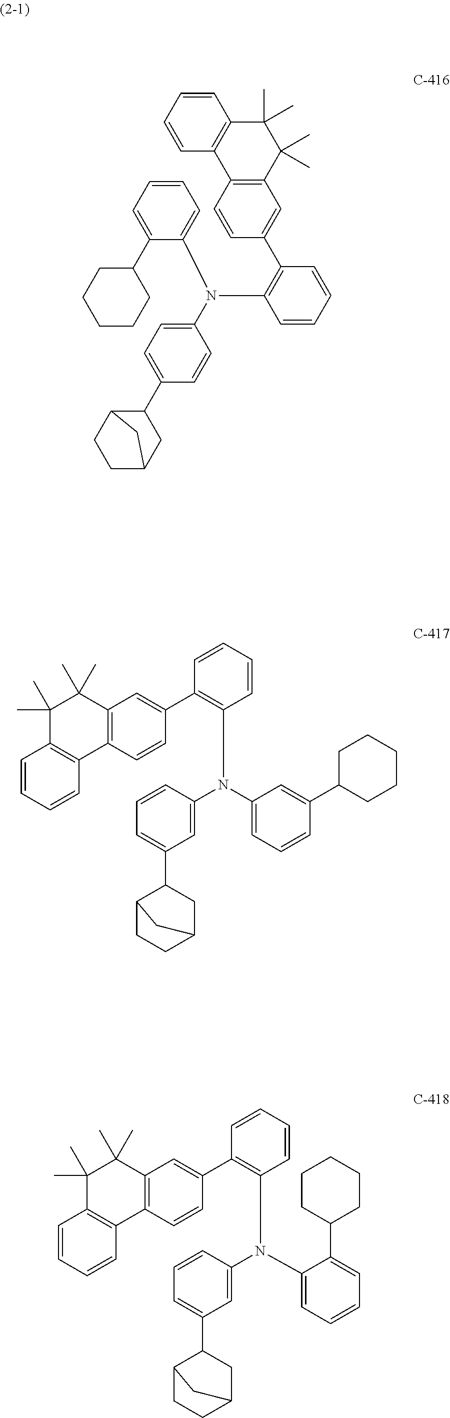 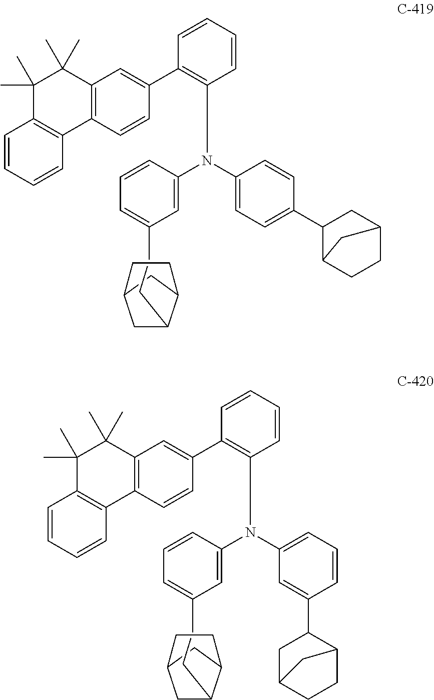 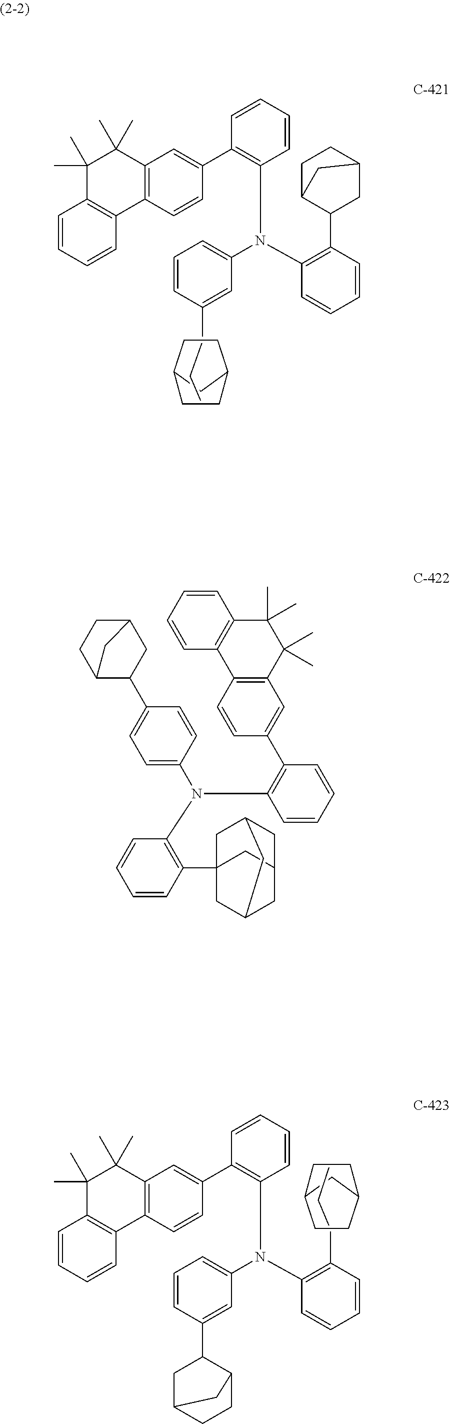 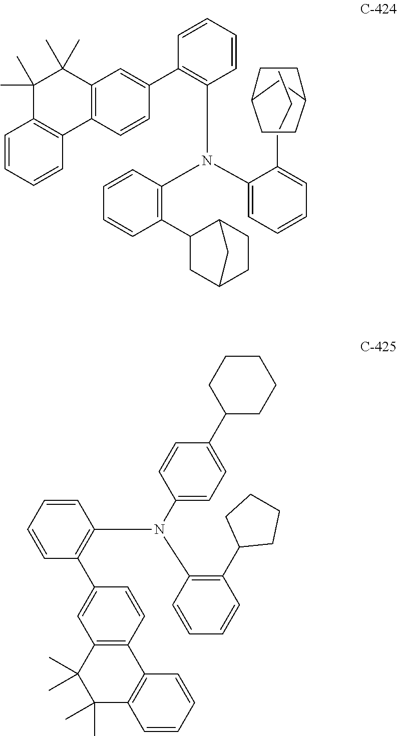 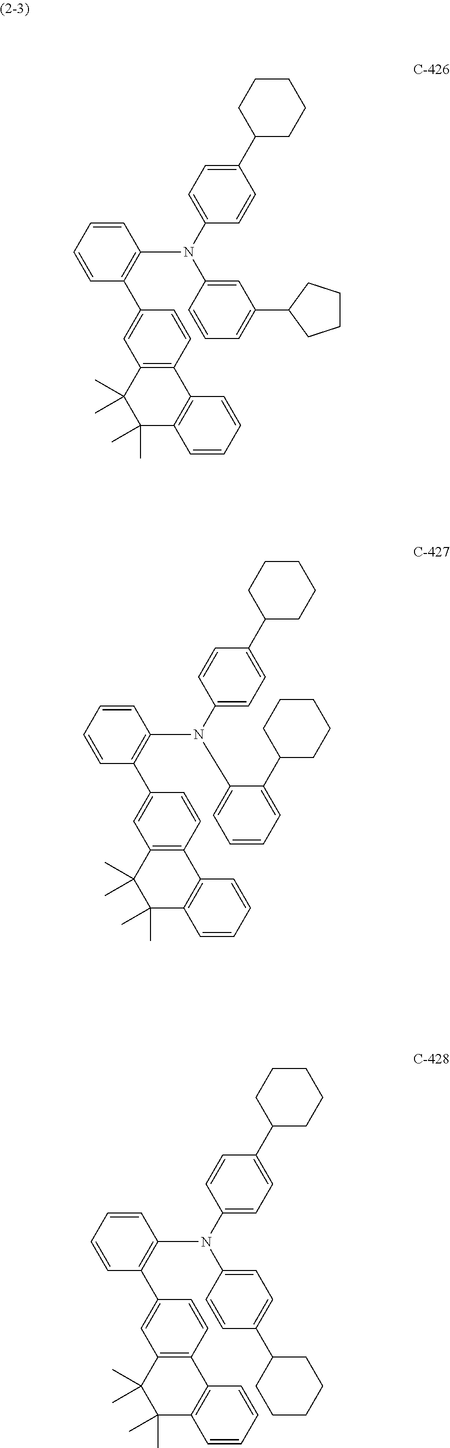 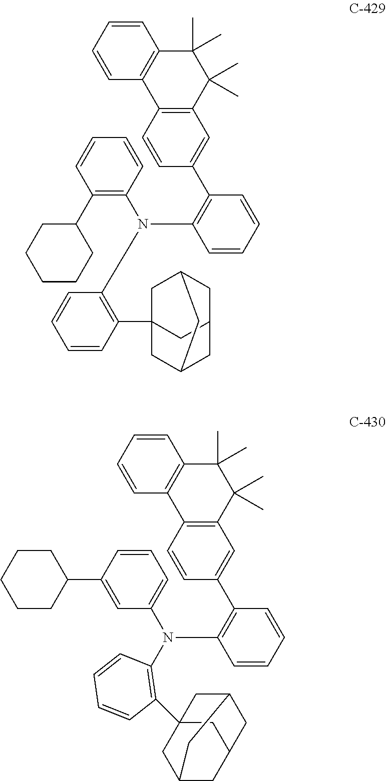 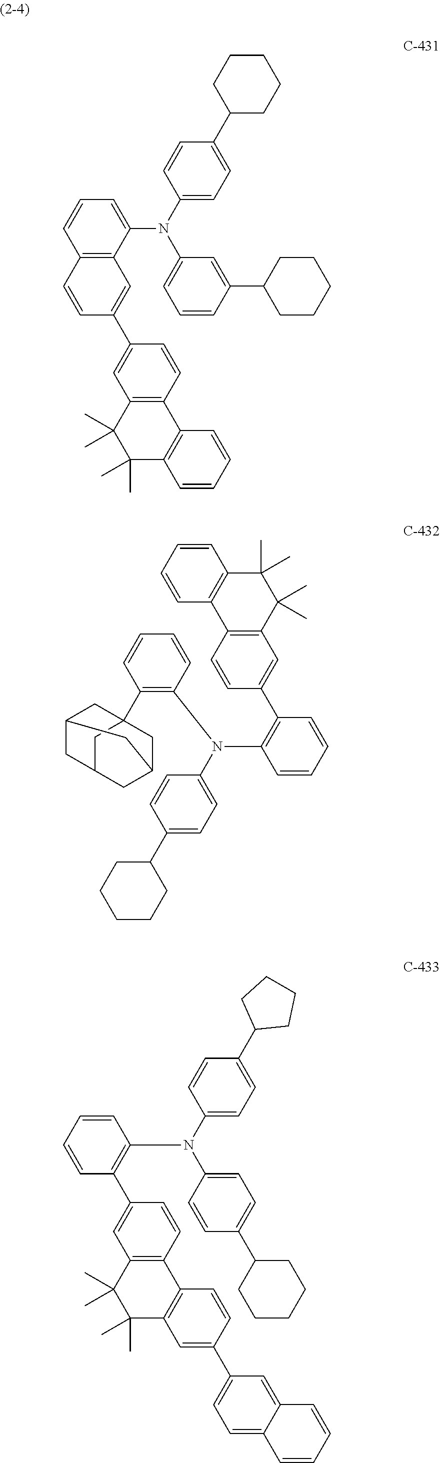 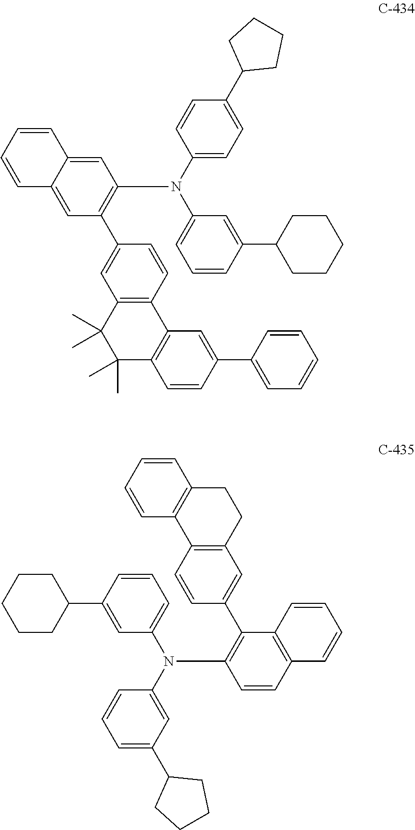 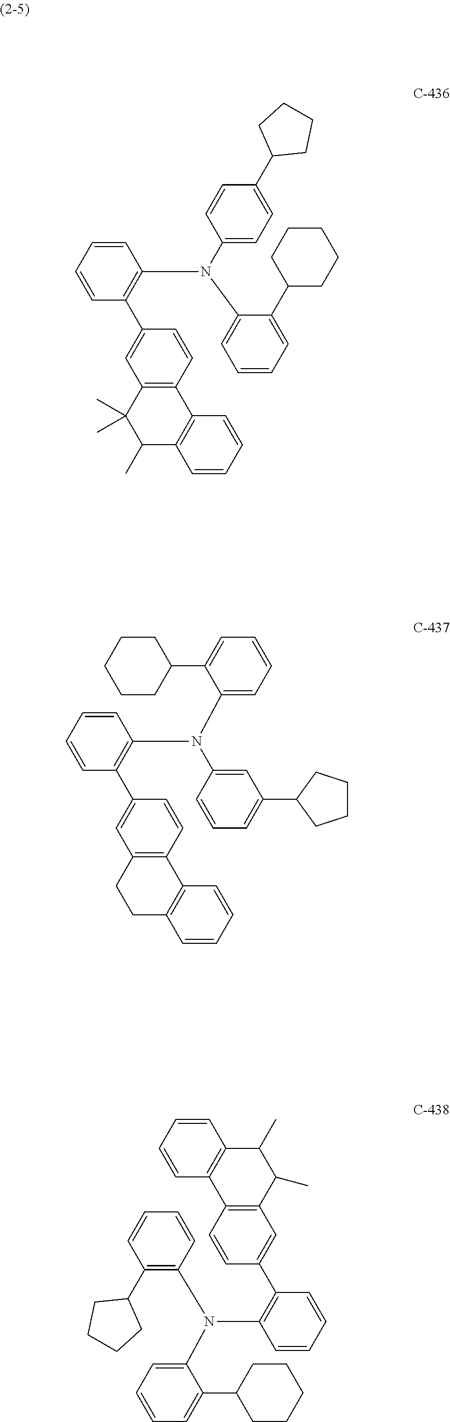 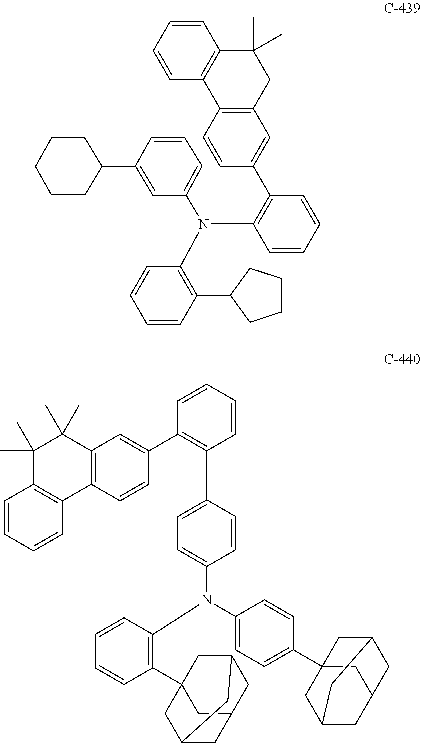 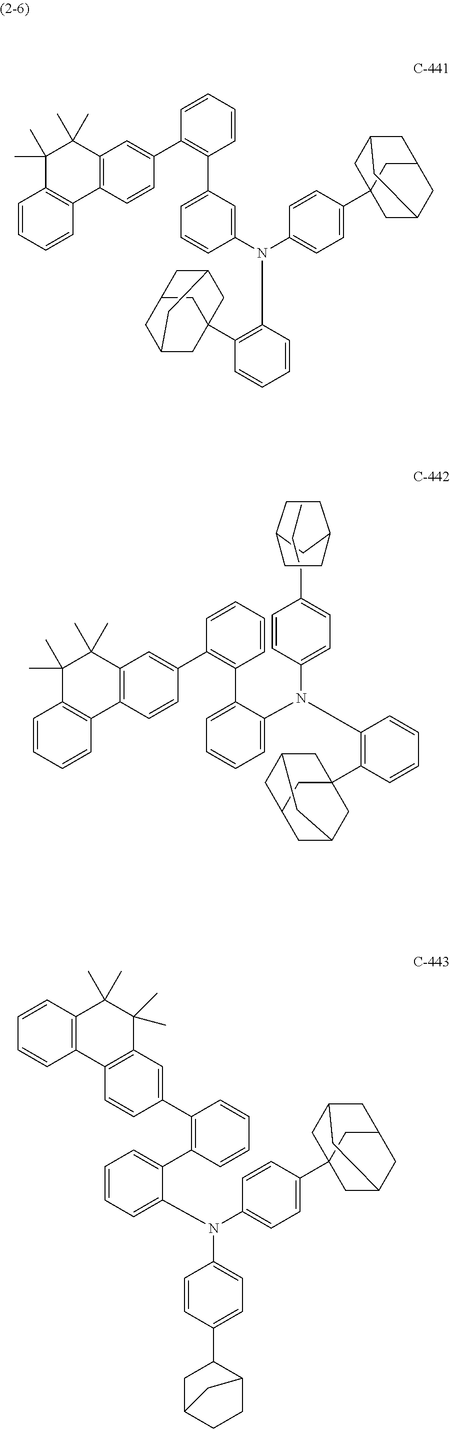 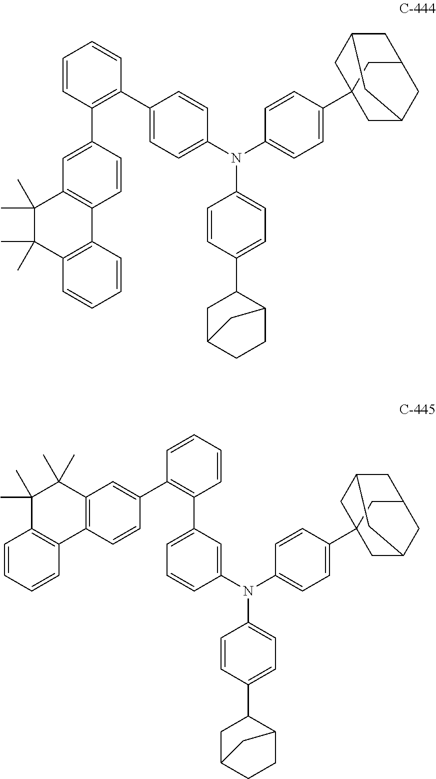 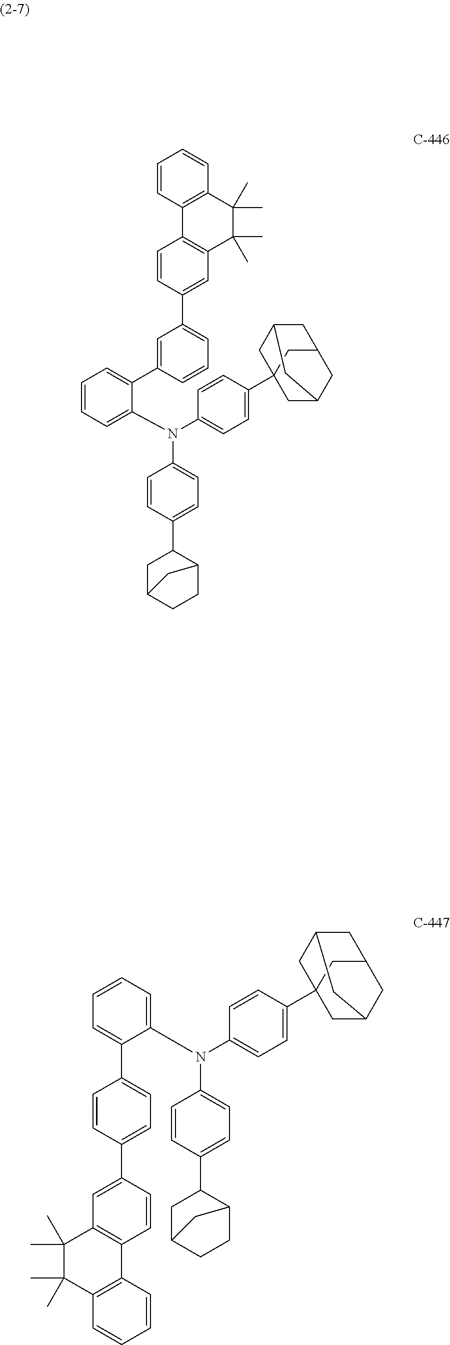 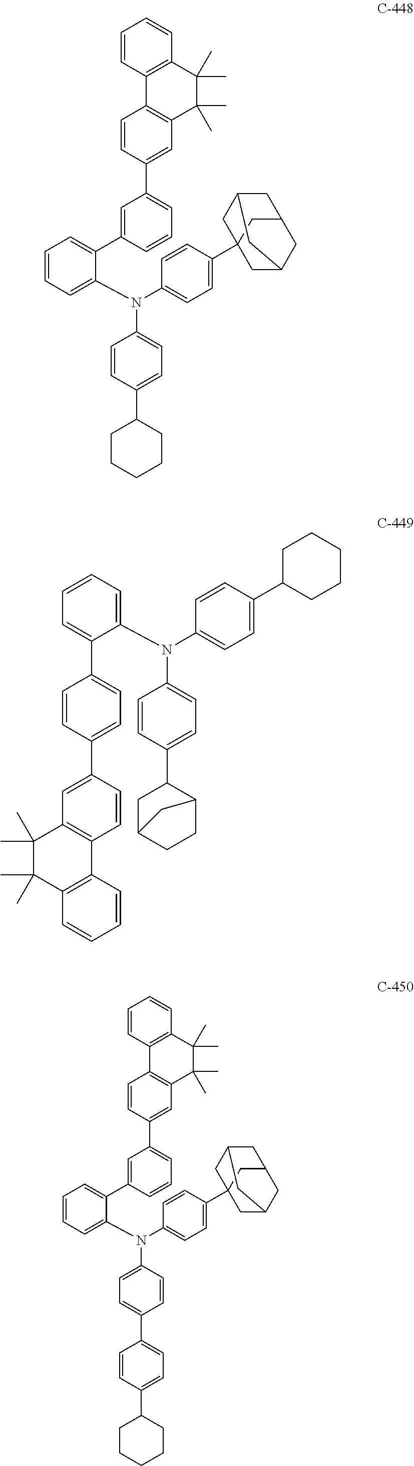 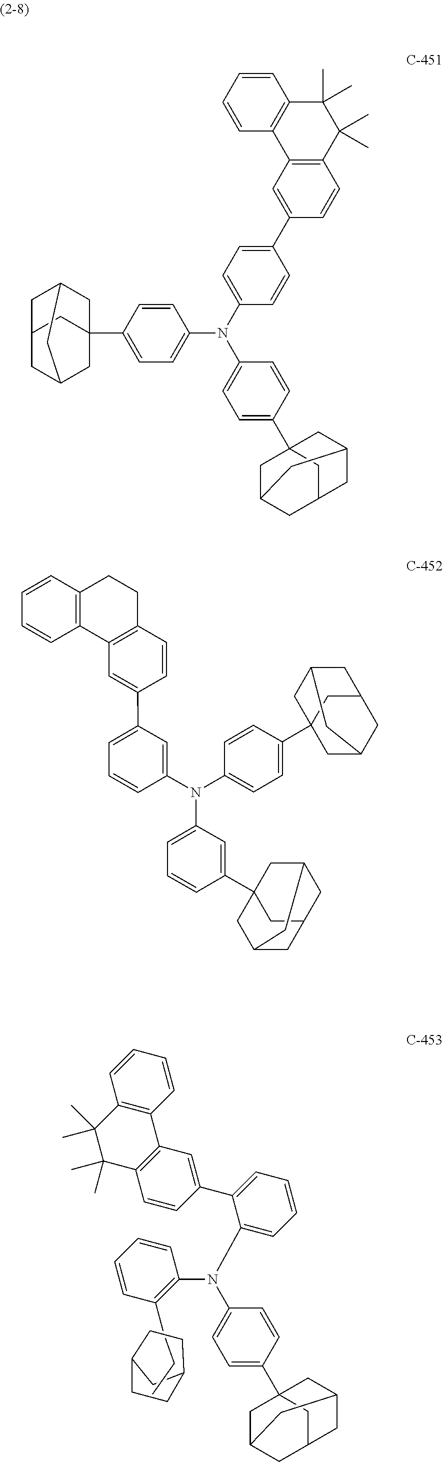 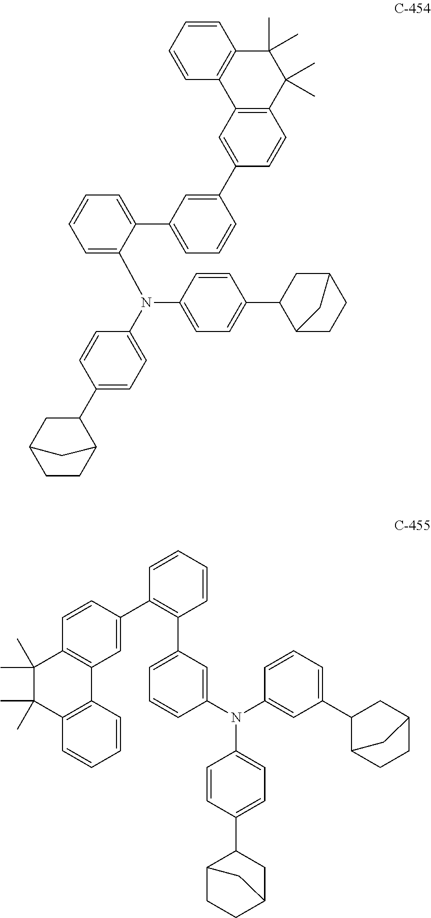 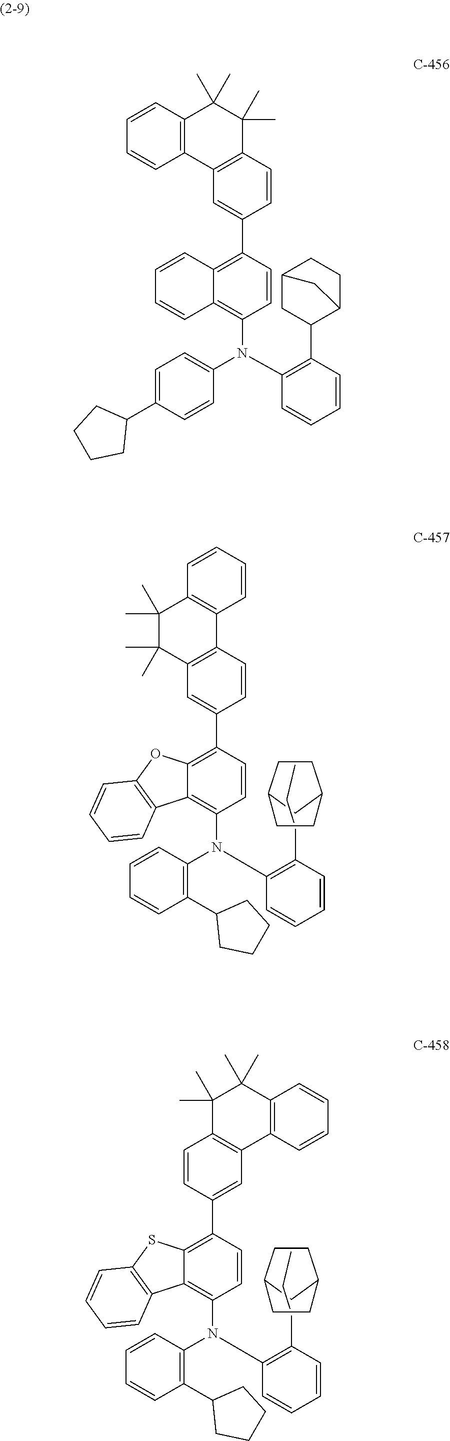 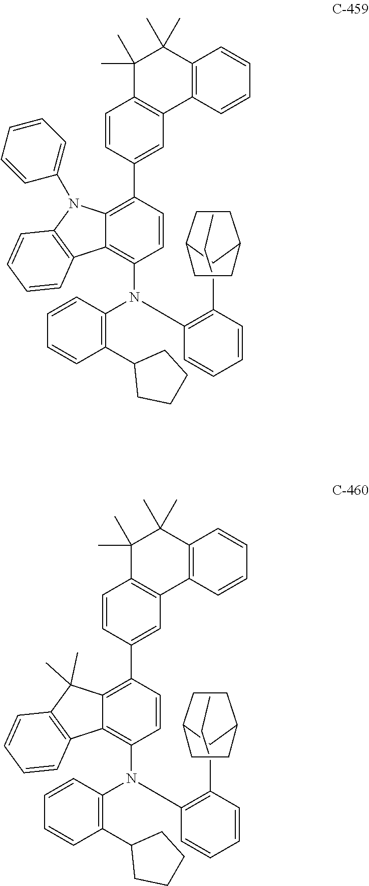 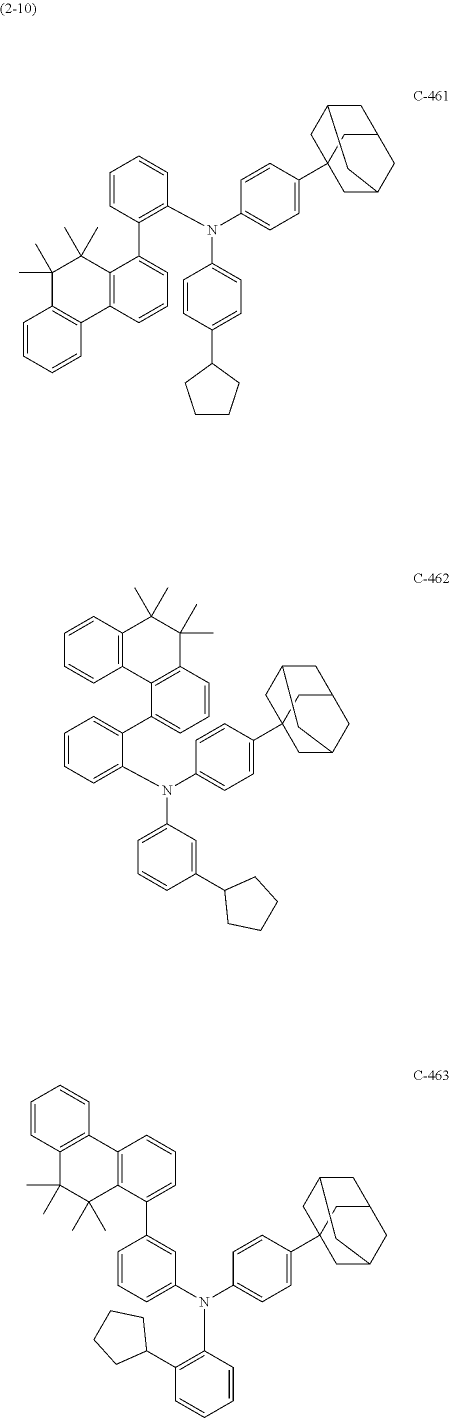 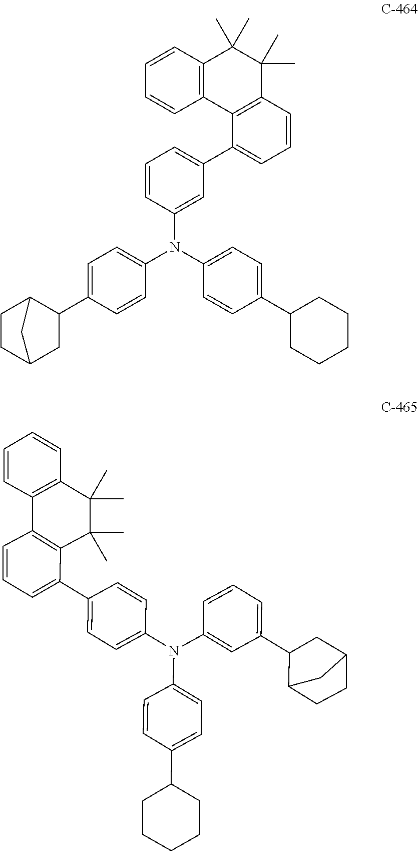 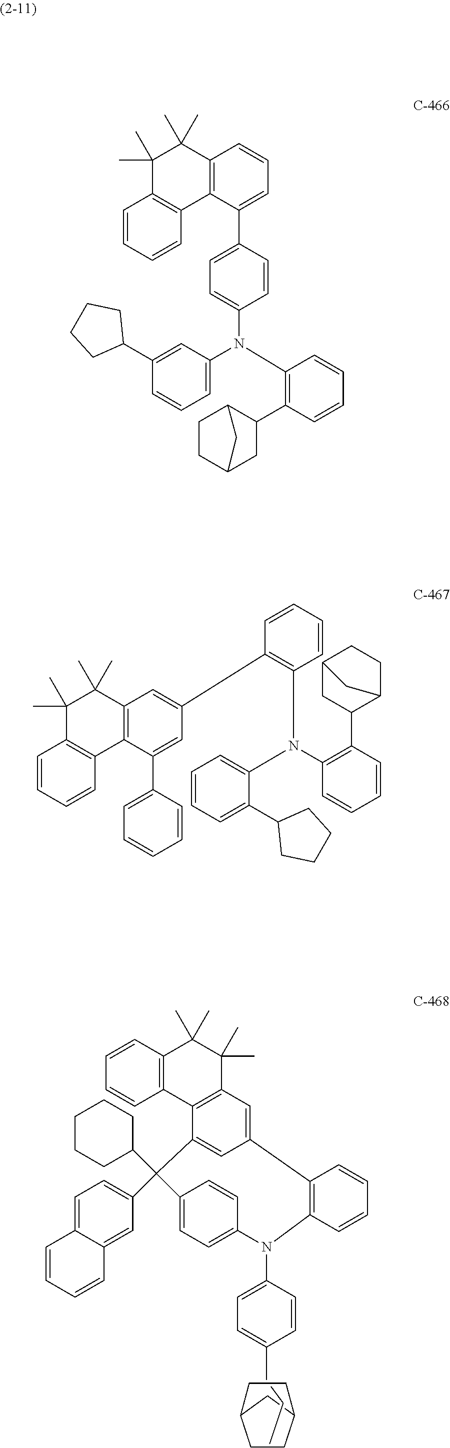 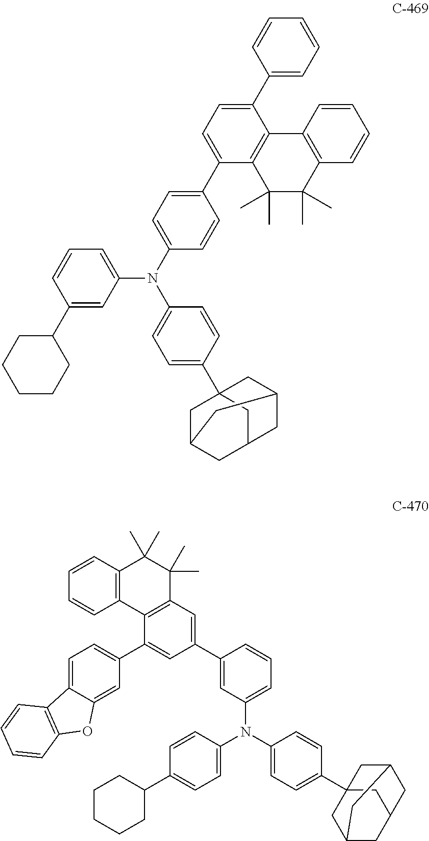
wherein,
R₁ to R₄ are as defined in claim 1; and
R₅ to R₂₀ are as defined for Rₐ in claim 1;
provided that at least one of R₁ to R₂₀ is represented by Formula 1-1 in claim 1.

4. The organic electroluminescent compound according to claim 1, wherein Formula 1 is represented by any one of the following Formulas 3-1 to 3-7: [chemical structure] [chemical structure] 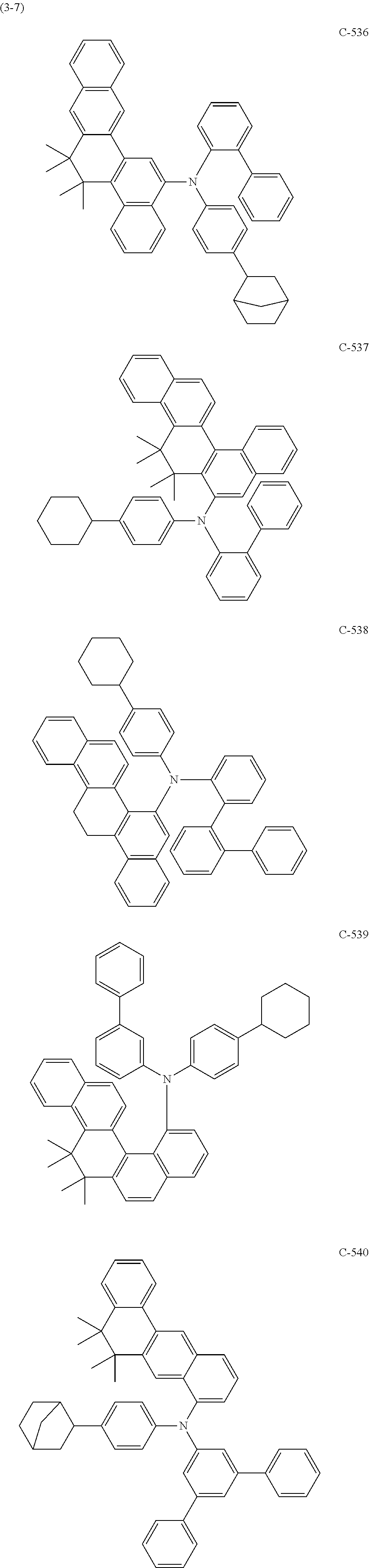
wherein,
R₁ to R₄ are as defined in claim 1; and
R₅ to R₁₆, R₂₁ to R₂₄, and R_{c} to R_{f} are as defined for Rₐ in claim 1;
provided that at least one of R₁ to R₁₆, R₂₁ to R₂₄ and R_{c} to R_{f} is represented by Formula 1-1 in claim 1.

5. The organic electroluminescent compound according to claim 1, wherein Formula 1 is represented by any one of the following Formulas 4-1 to 4-6: 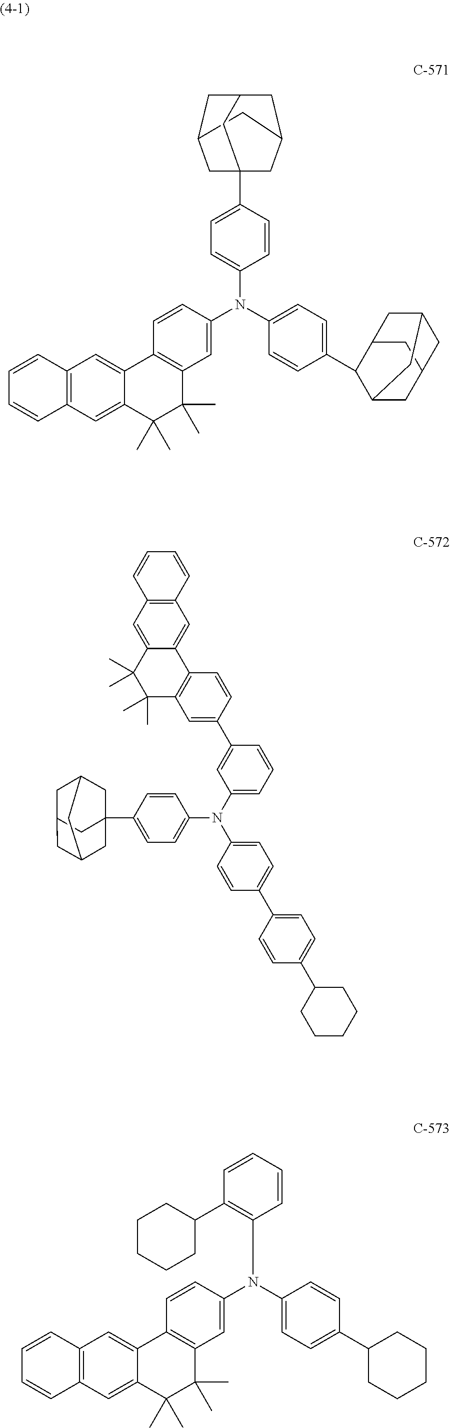 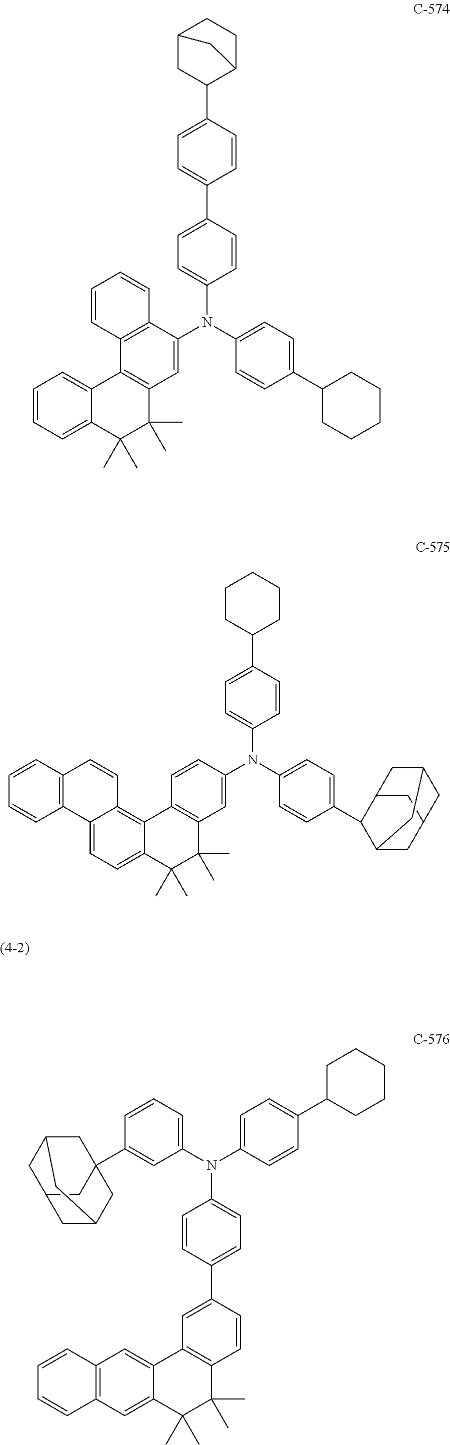 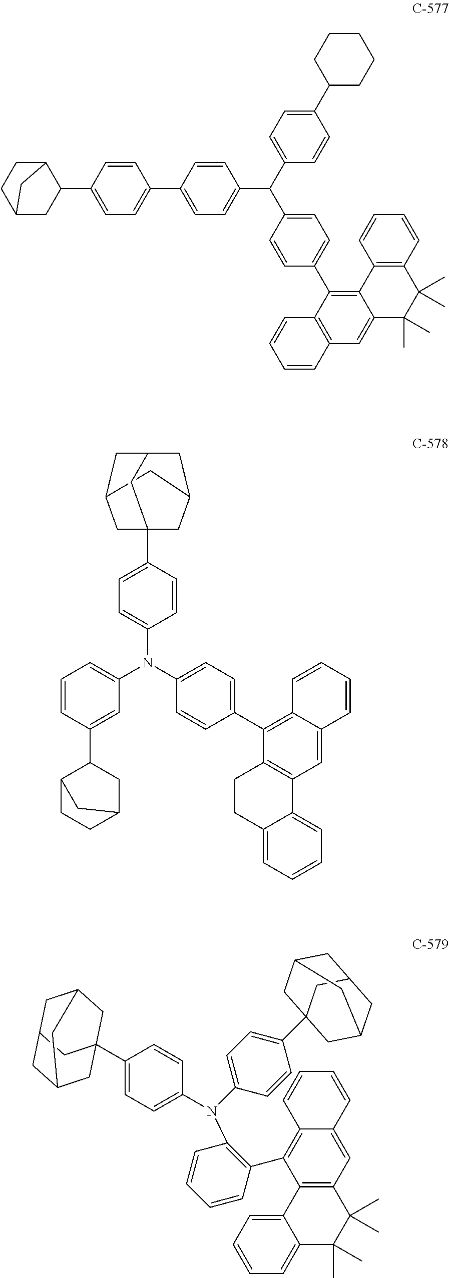 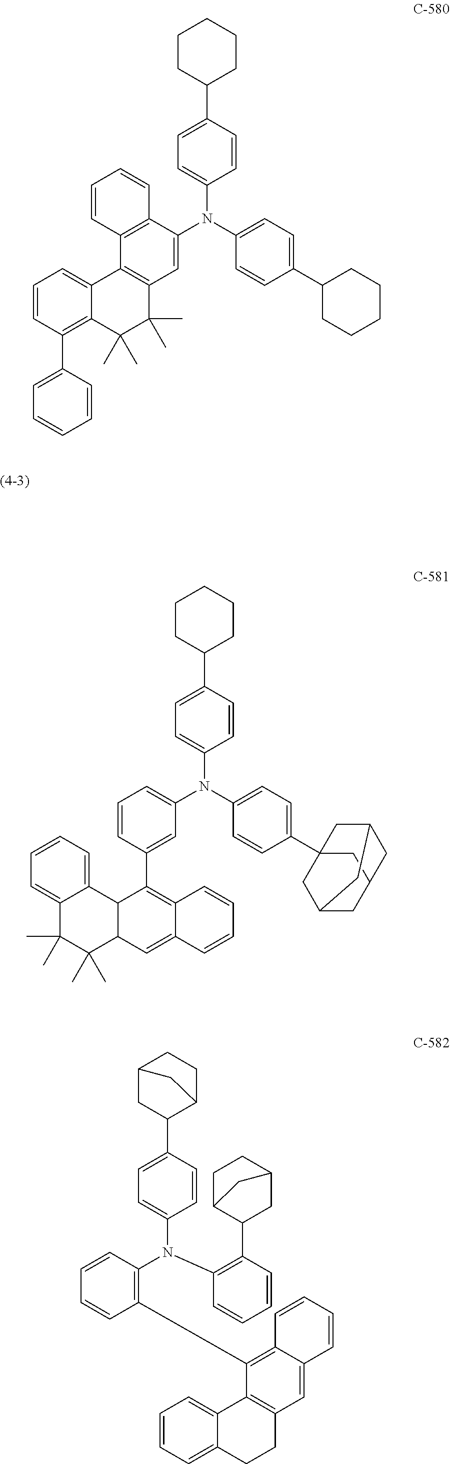 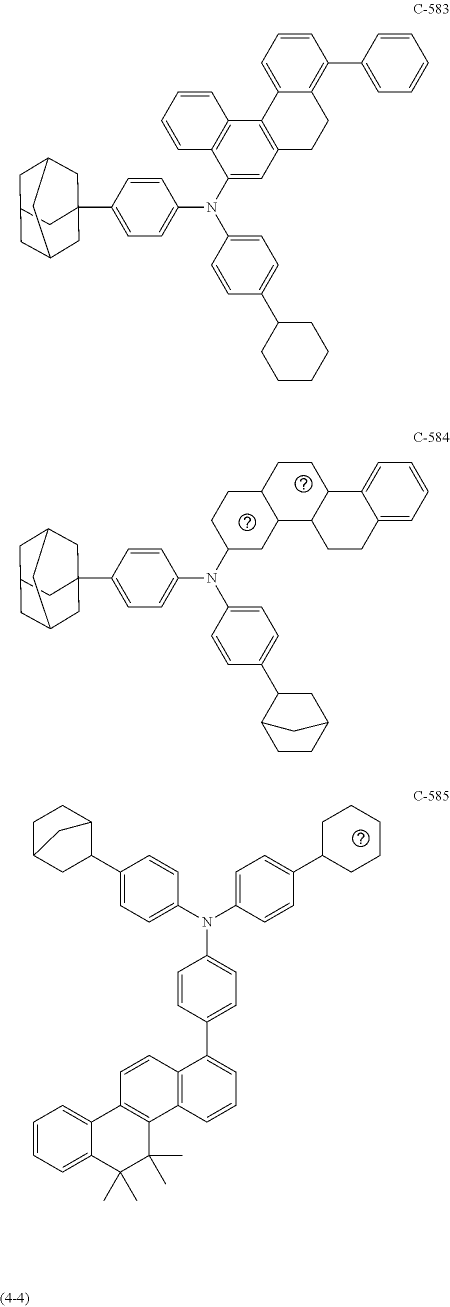 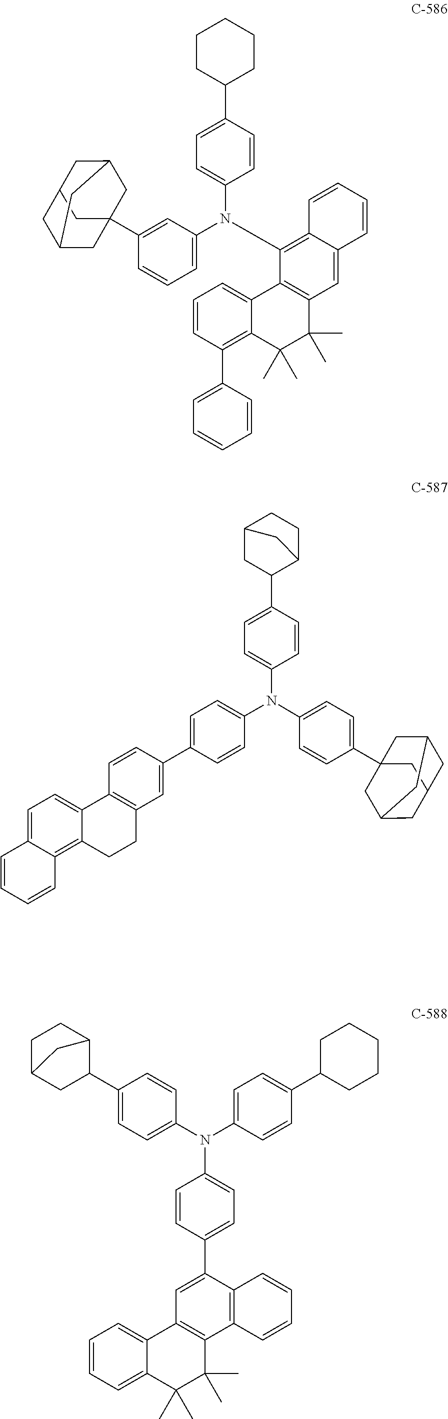 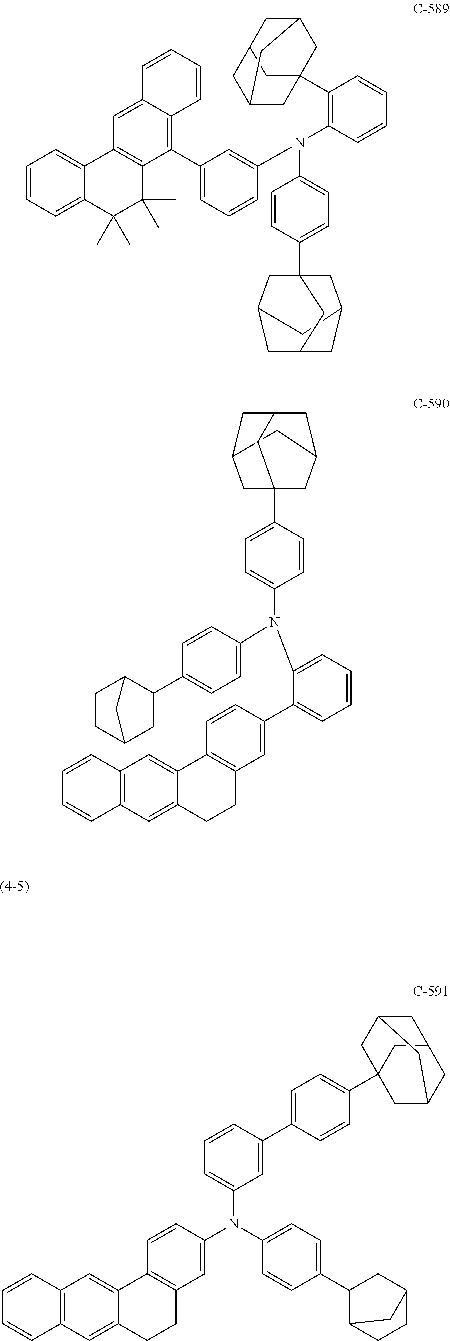 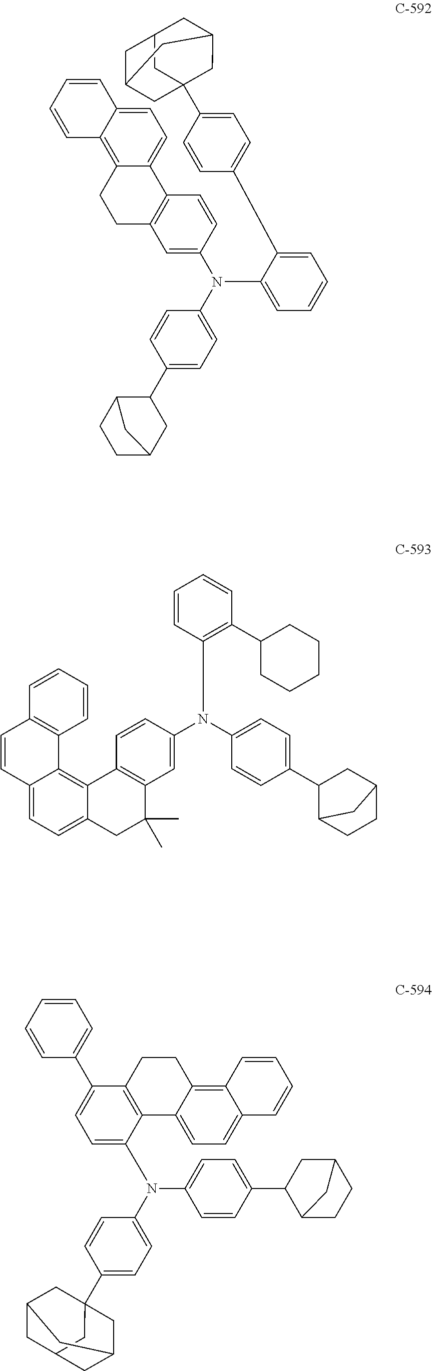 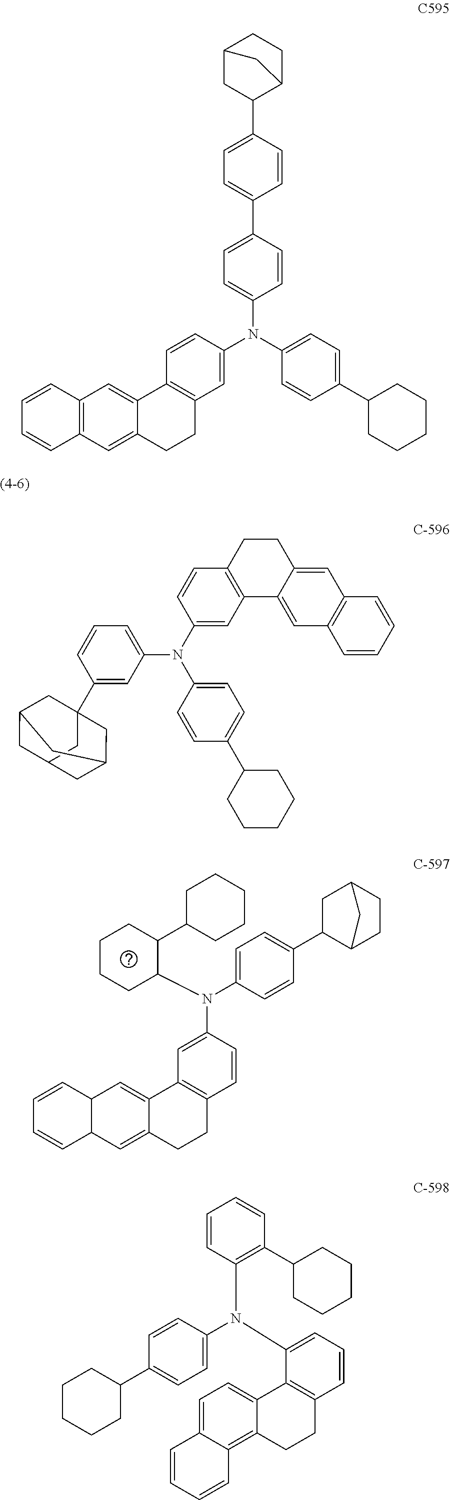 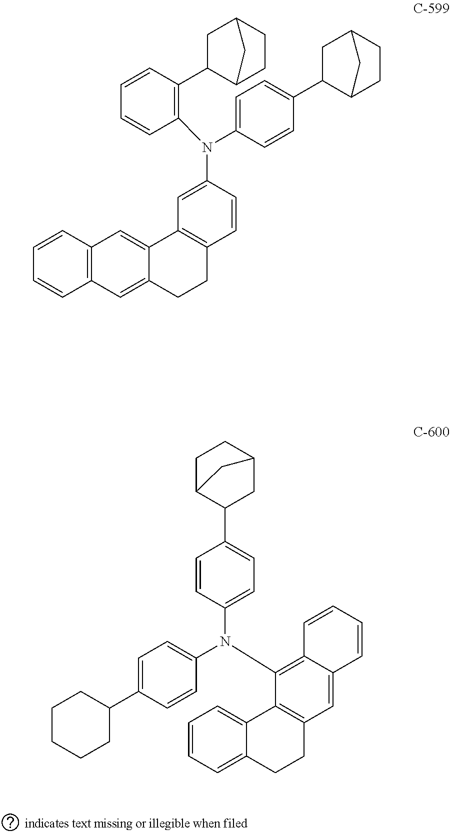
wherein,
X represents NR₂₉, O, S, or CR₃₀R₃₁;
R₁ to R₄ are as defined in claim 1; and
R₅ to R₁₂ and R₂₅ to R₃₁ are as defined for Rₐ in claim 1;
provided that at least one of R₁ to R₁₂ and R₂₅ to R₃₁ is represented by Formula 1-1 in claim 1.

6. The organic electroluminescent compound according to claim 1, wherein R₁ to R₄ each independently represent hydrogen, deuterium, a substituted or unsubstituted (C₁-C₃₀)alkyl, a substituted or unsubstituted (C₆-C₃₀)aryl, a substituted or unsubstituted (3- to 30-membered)heteroaryl, or a substituted or unsubstituted (C₃-C₃₀)cycloalkyl.

7. The organic electroluminescent compound according to claim 1, wherein at least one of Ar₁ and Ar₂ represents cyclopropyl, cyclobutyl, cyclopentyl, cyclohexyl, cycloheptyl, cyclooctyl, bicyclo[2.1.1]hexyl, bicyclo[2.2.1]heptyl, bicyclo[3.1.1]heptyl, bicyclo[2.2.2]octyl, bicyclo[3.2.1]octyl, adamantanyl, 2,3-dihydro-1H-indenyl, 1,2,3,4-tetrahydronaphthalenyl, or 6,7,8,9-tetrahydro-5H-benzo[7]annulenyl.

8. The organic electroluminescent compound according to claim 1, wherein the organic electroluminescent compound represented by Formula 1 has a refractive index of 1.71 or less.

9. The organic electroluminescent compound according to claim 1, wherein the organic electroluminescent compound represented by Formula 1 is selected from the following compounds: 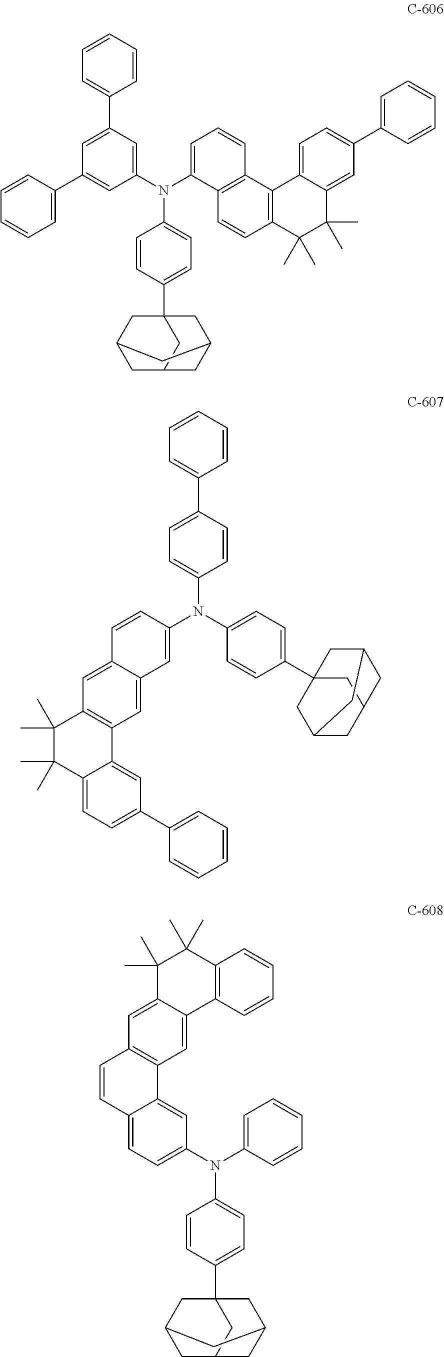 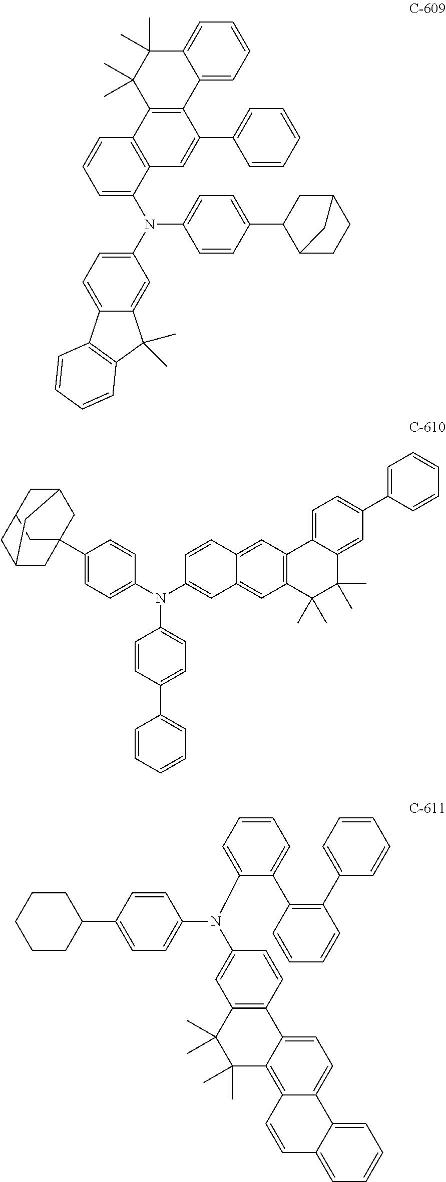 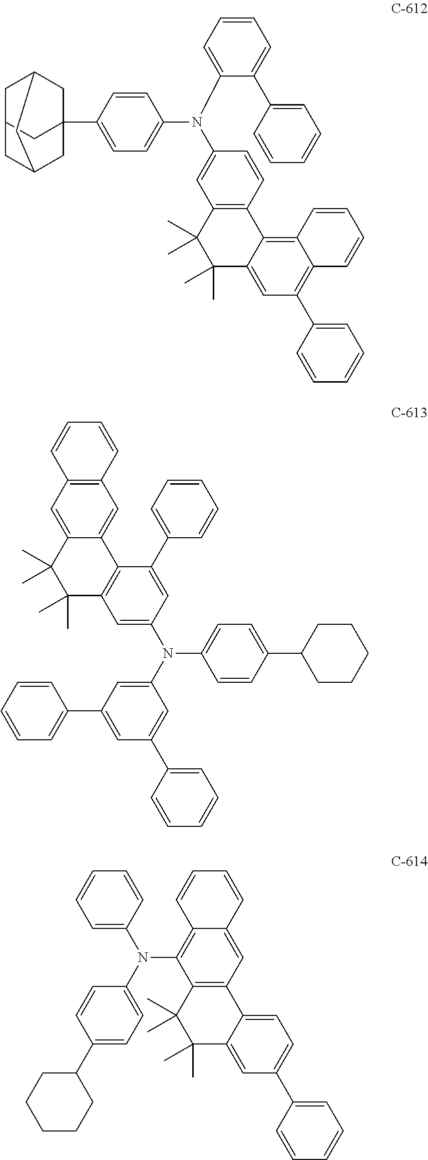 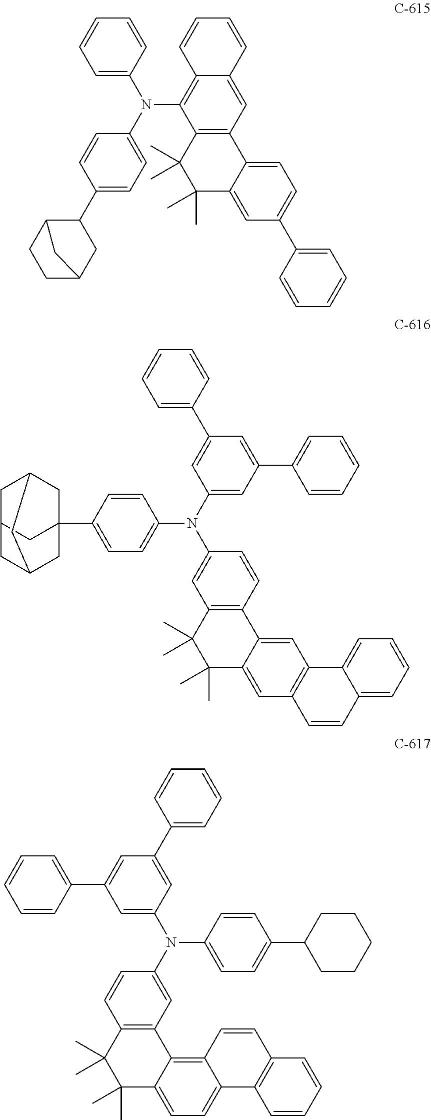 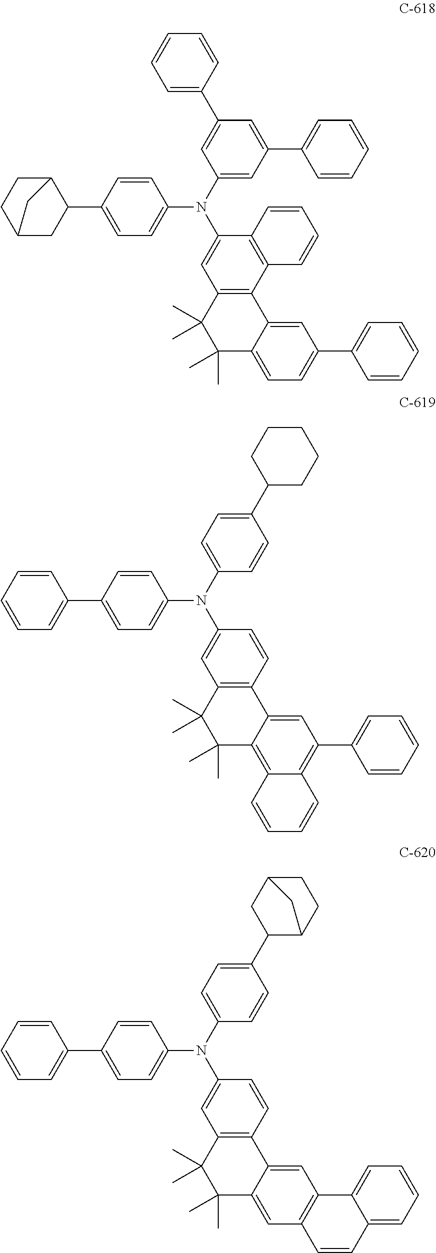 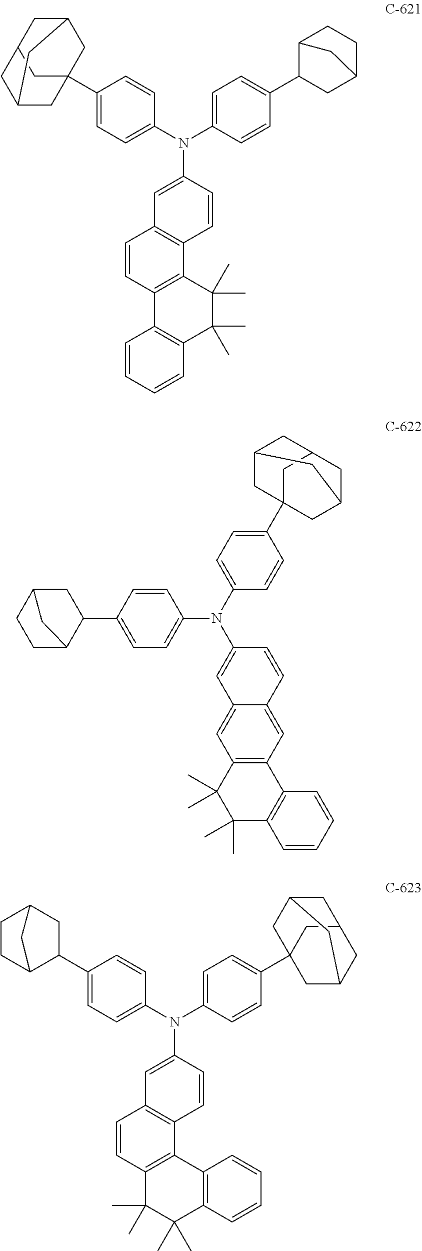 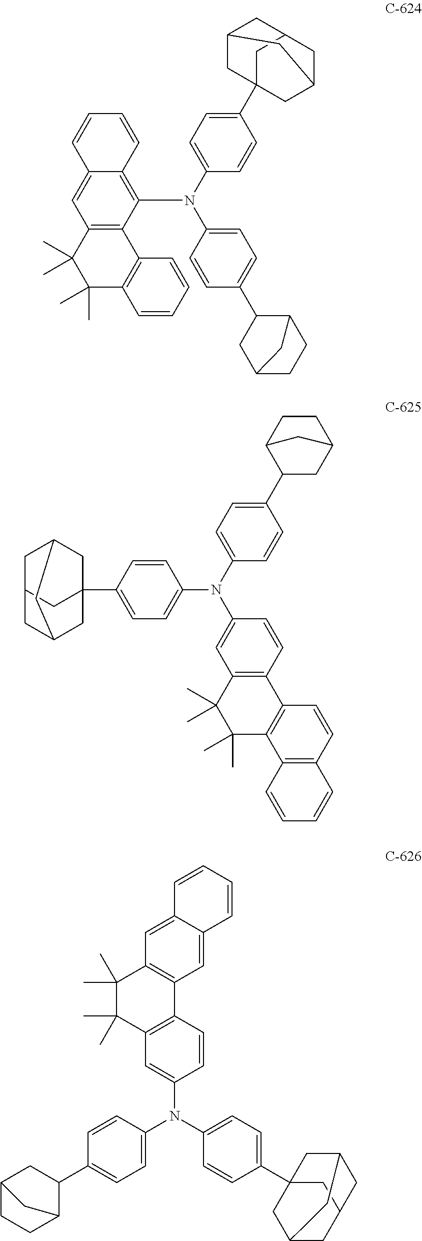 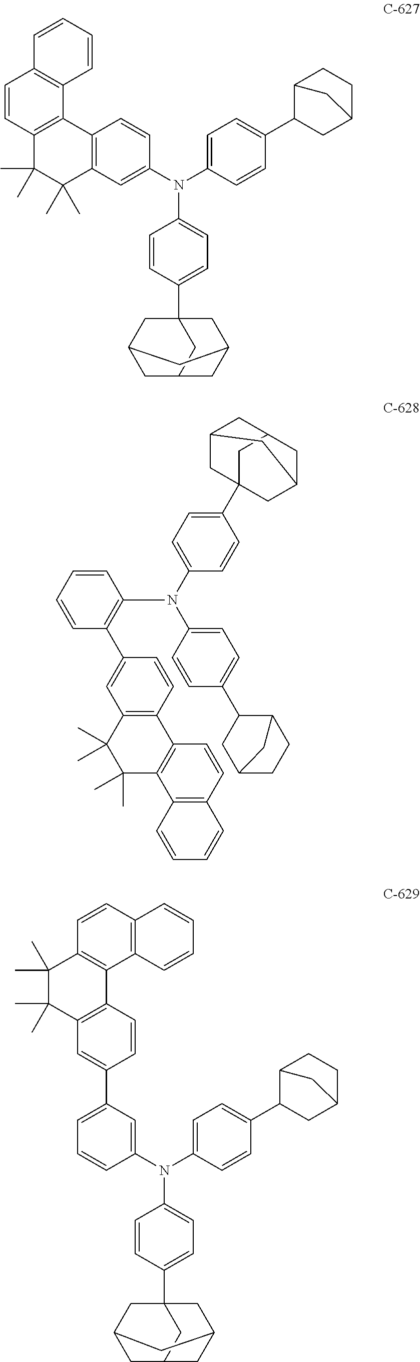 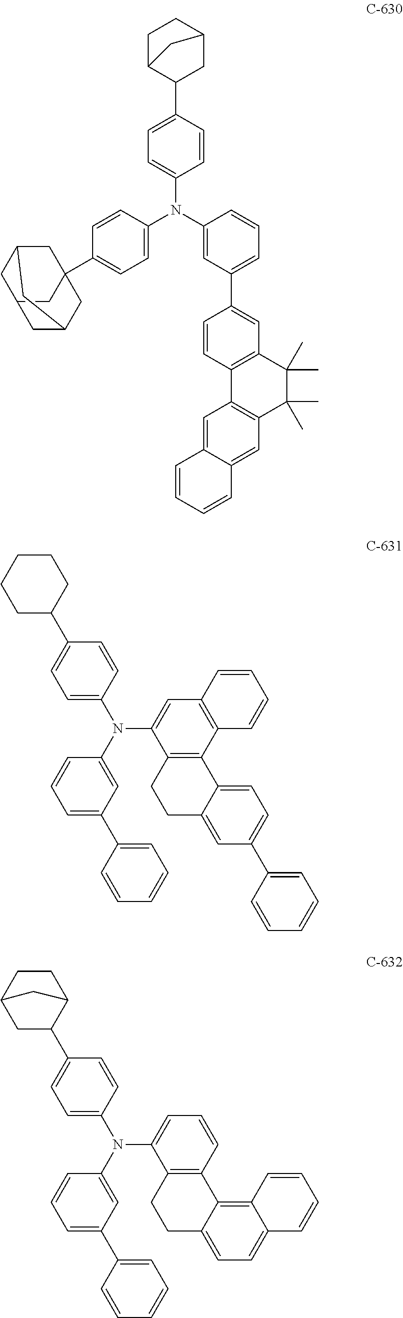 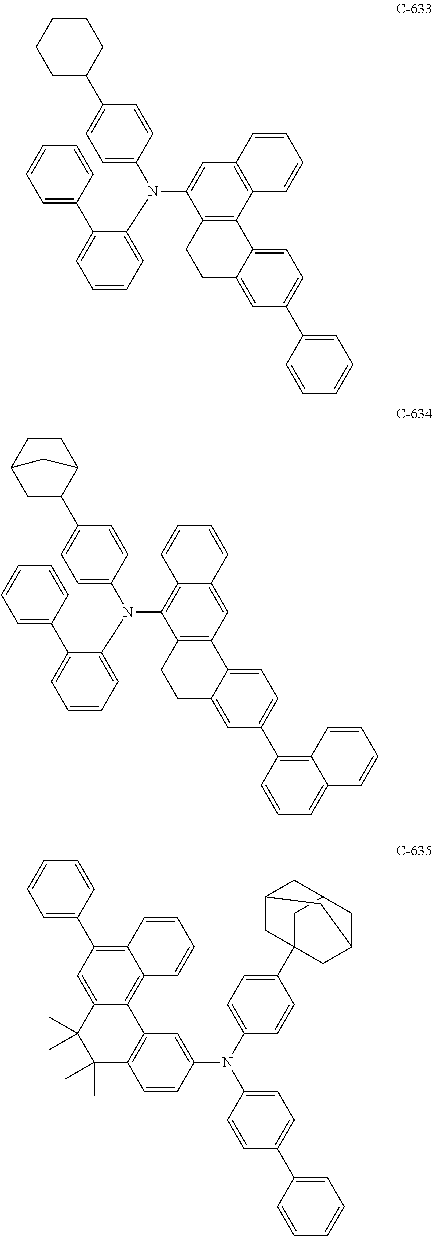 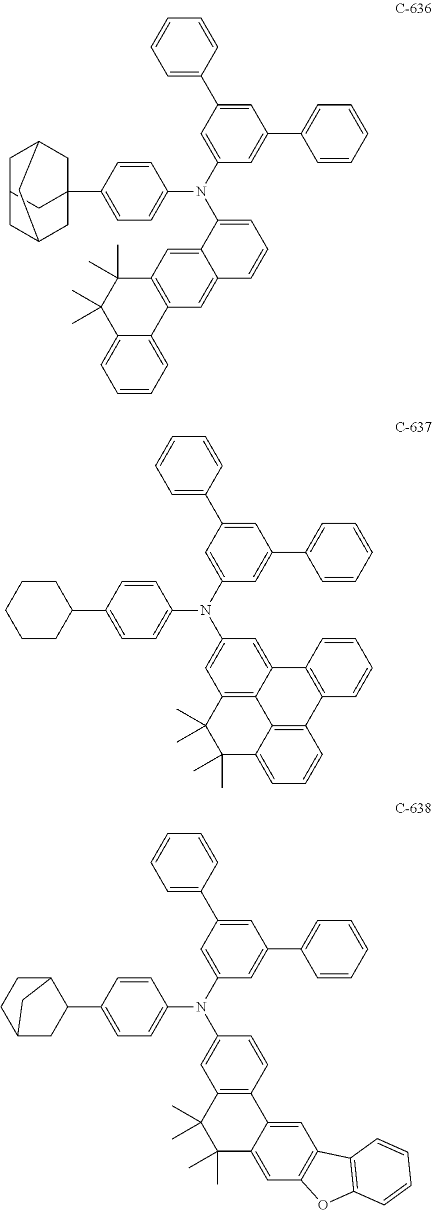 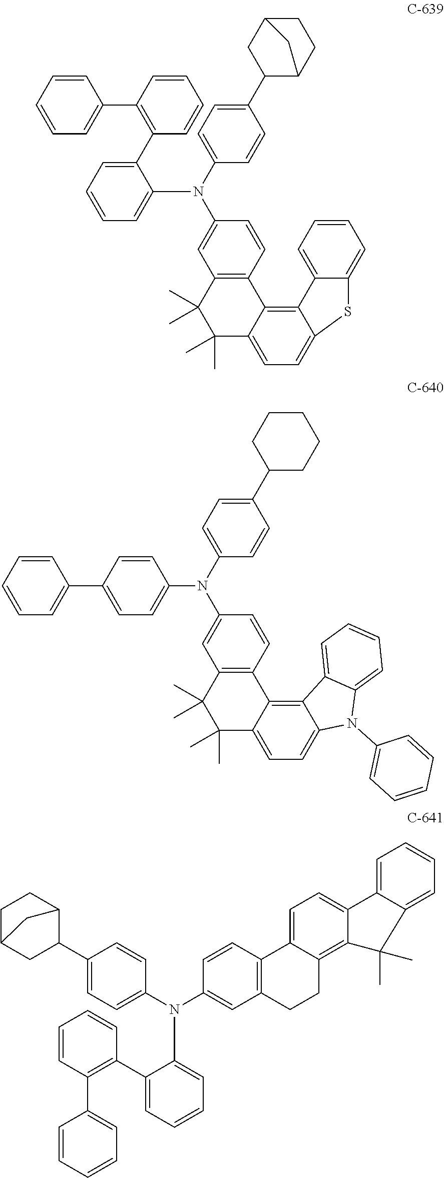 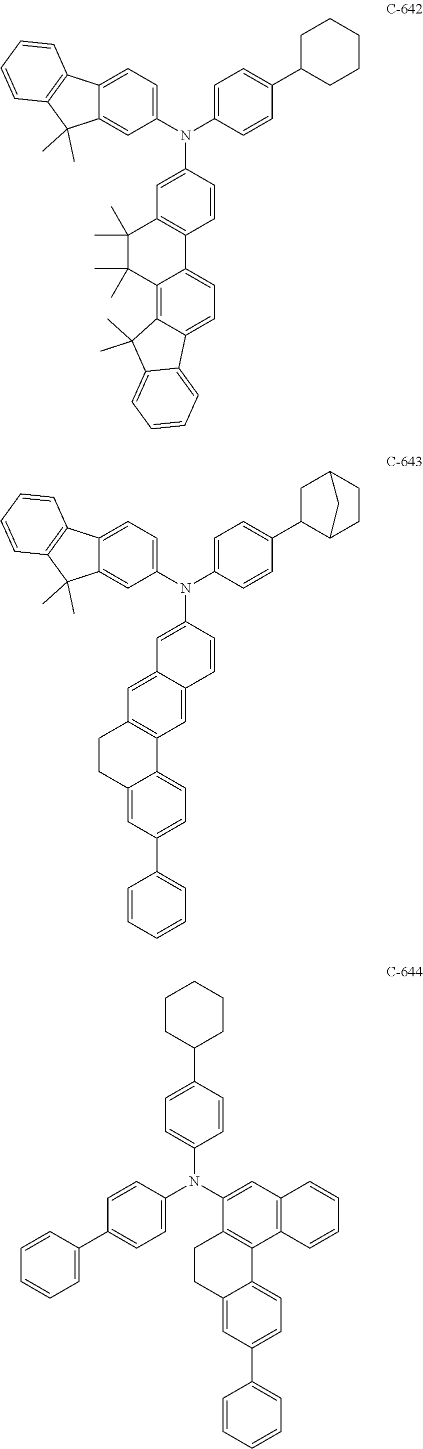 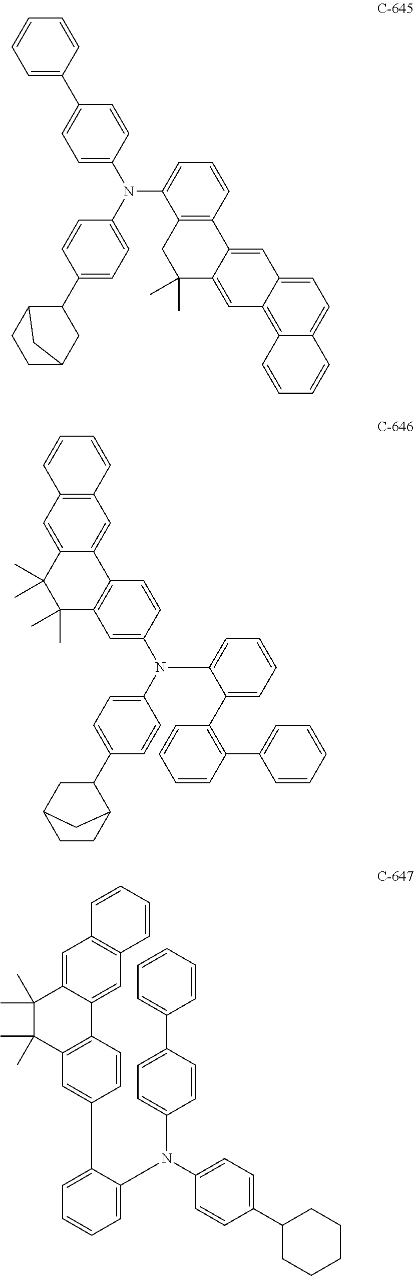 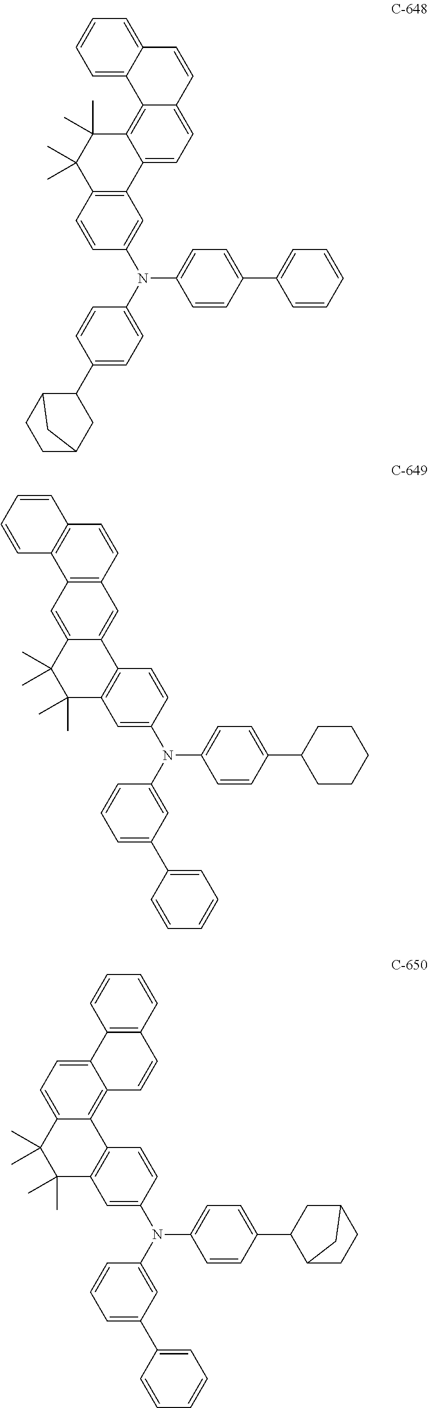 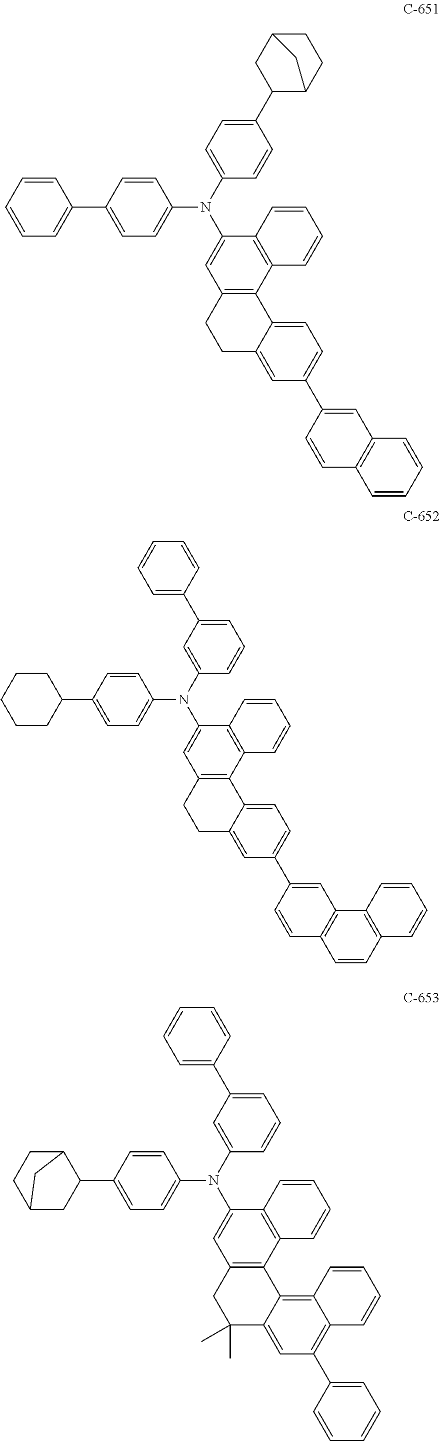 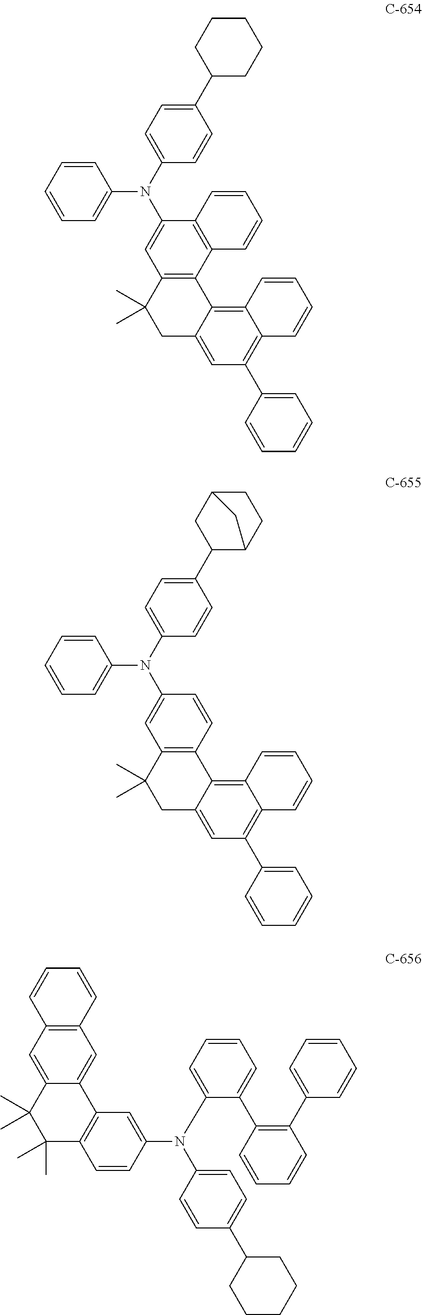 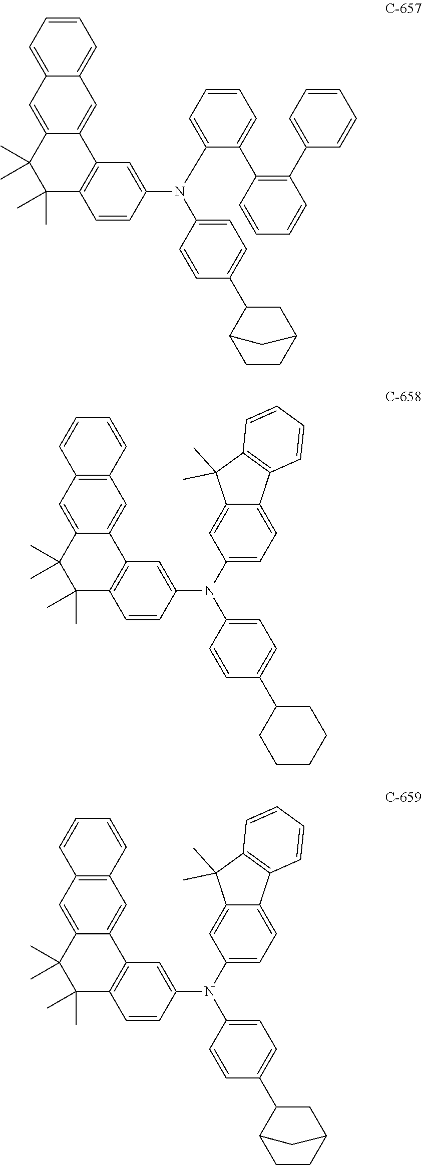 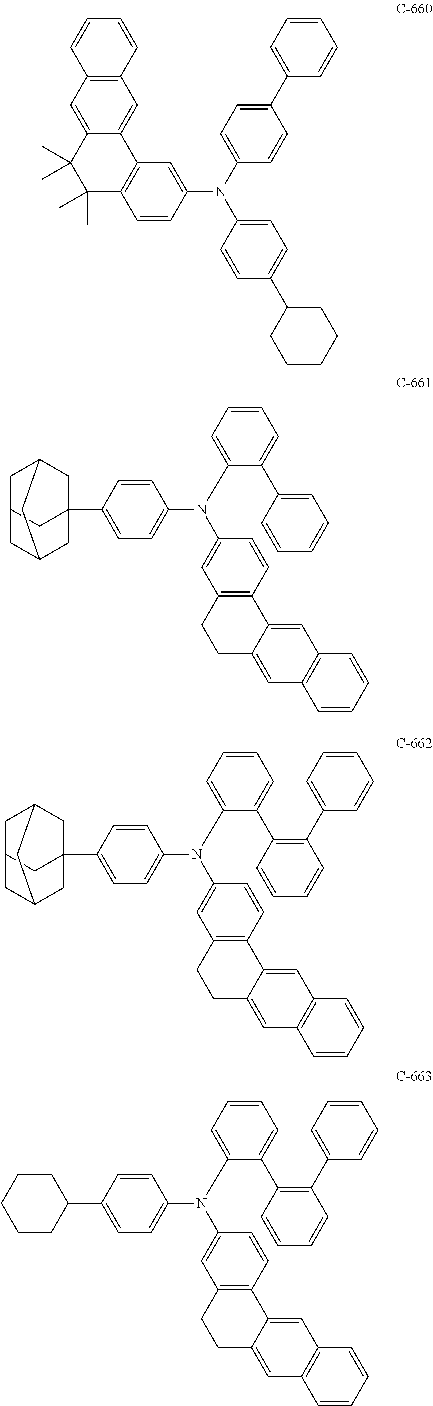 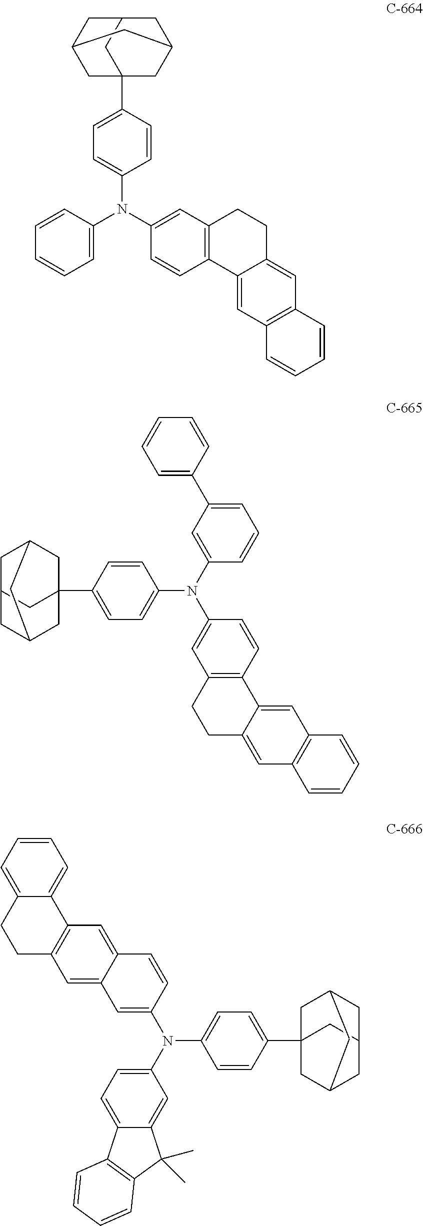 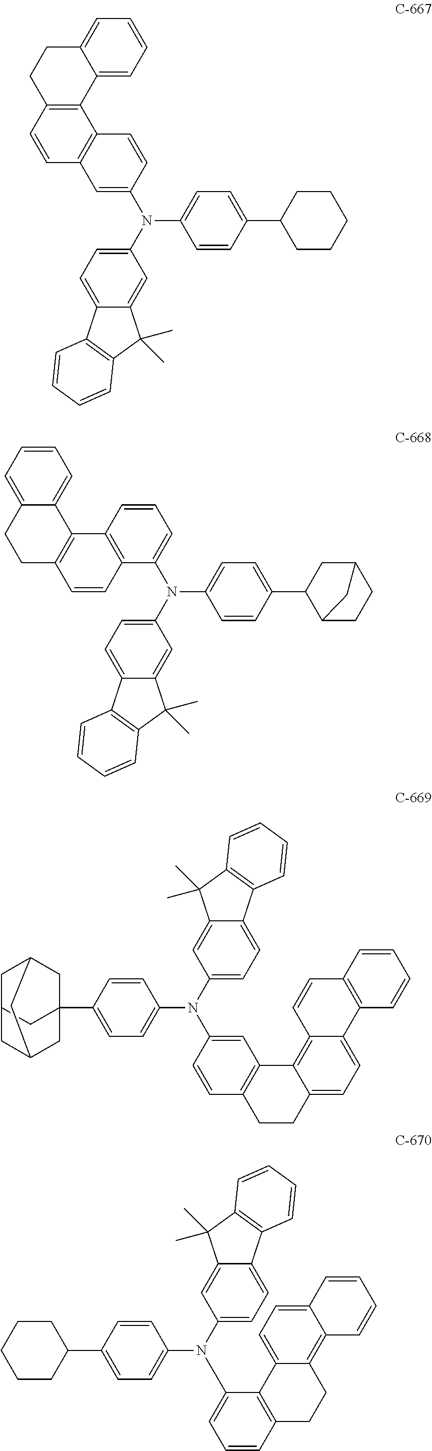 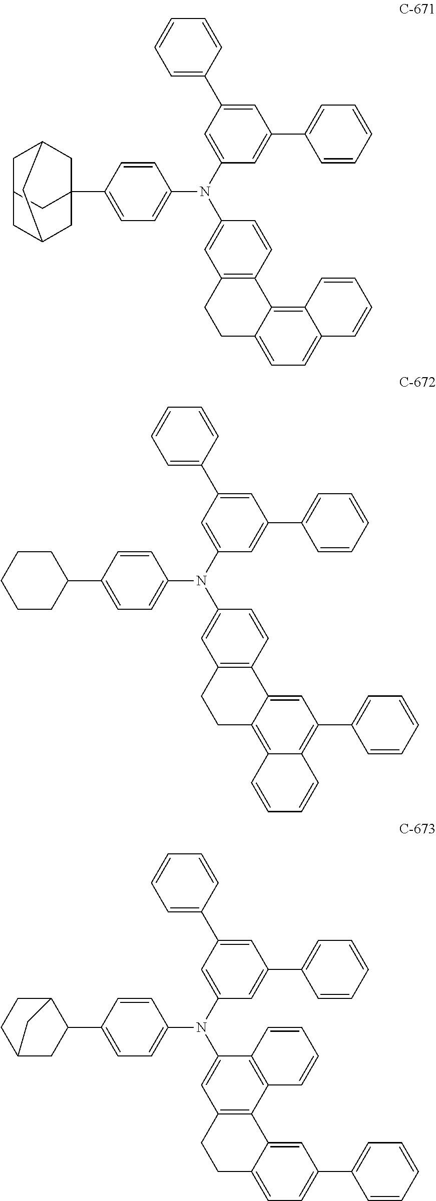 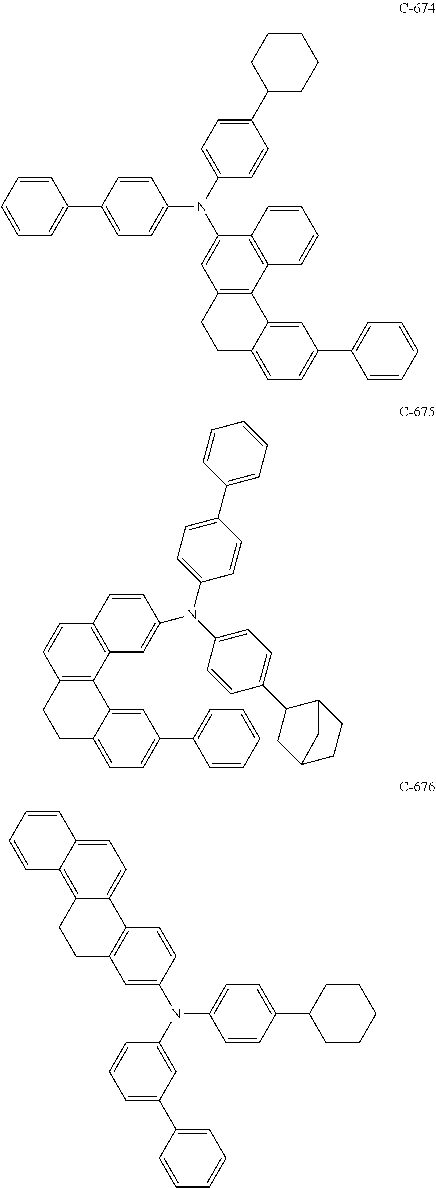 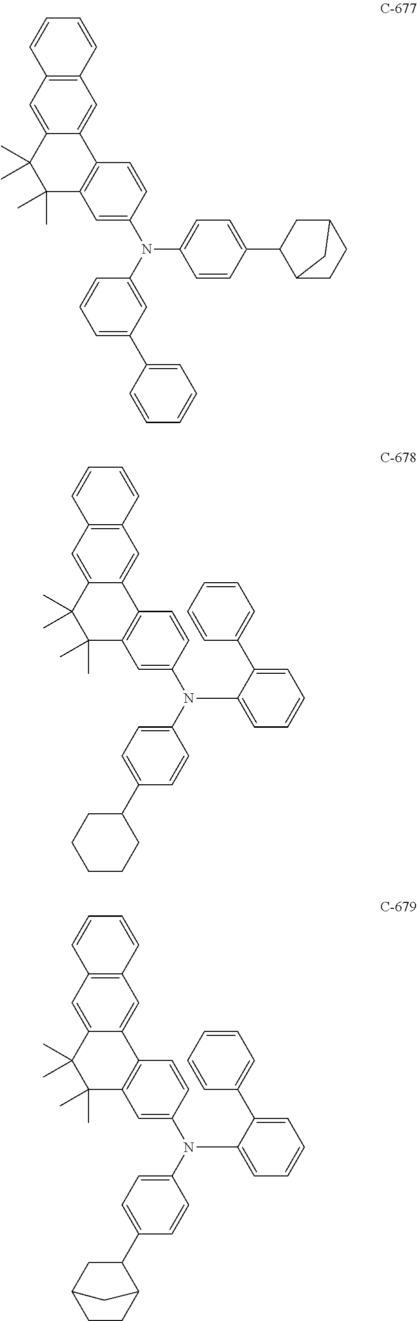 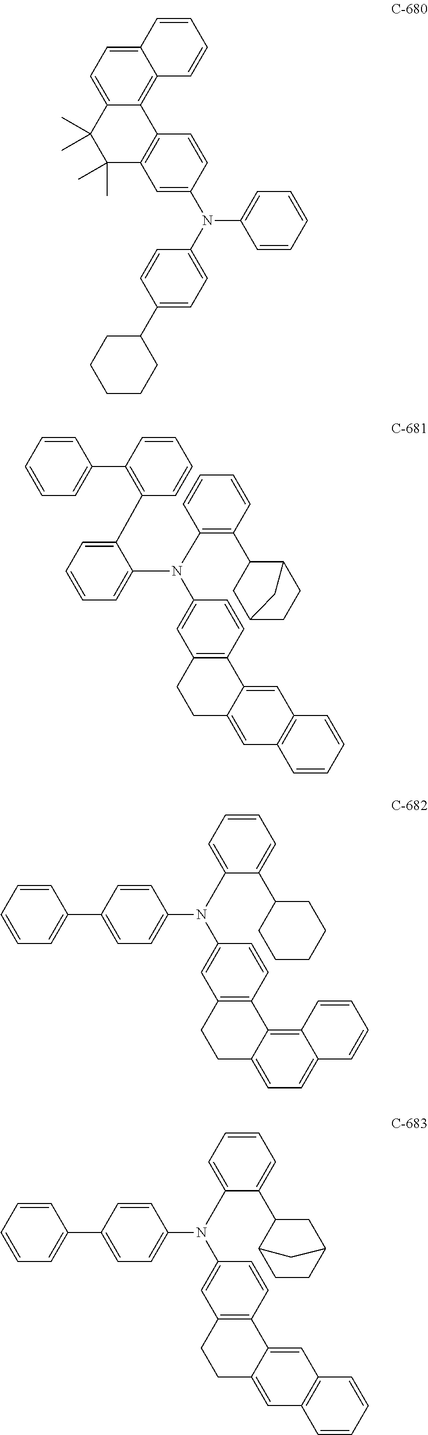 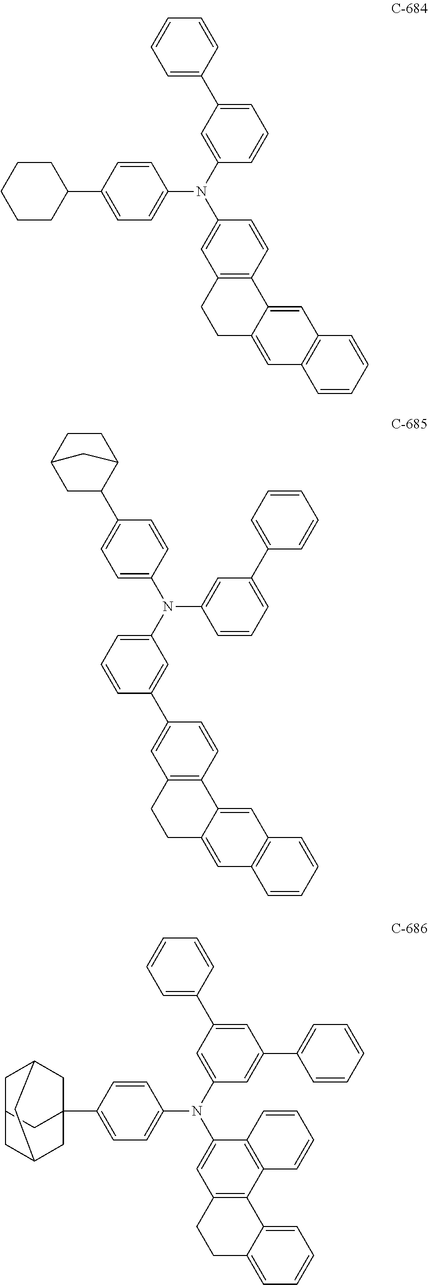 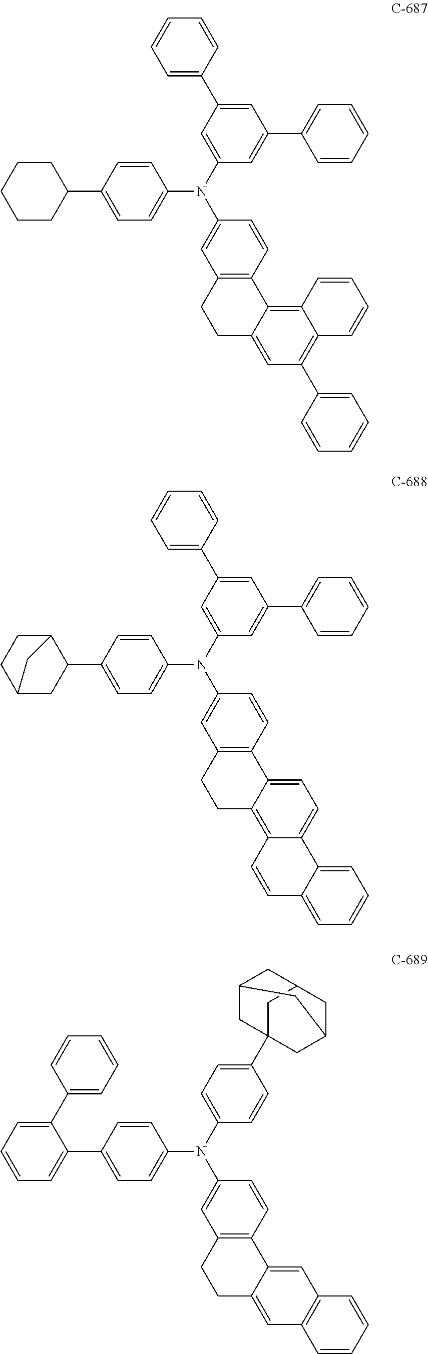 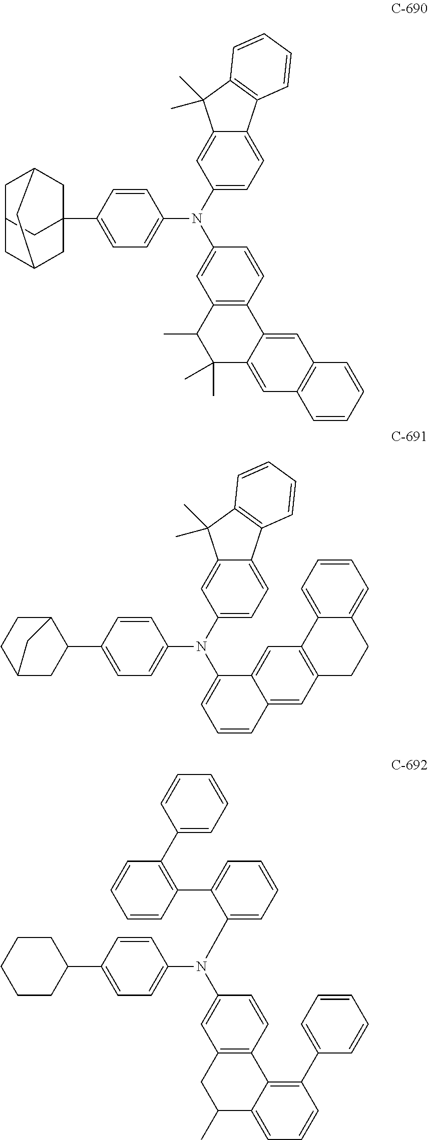 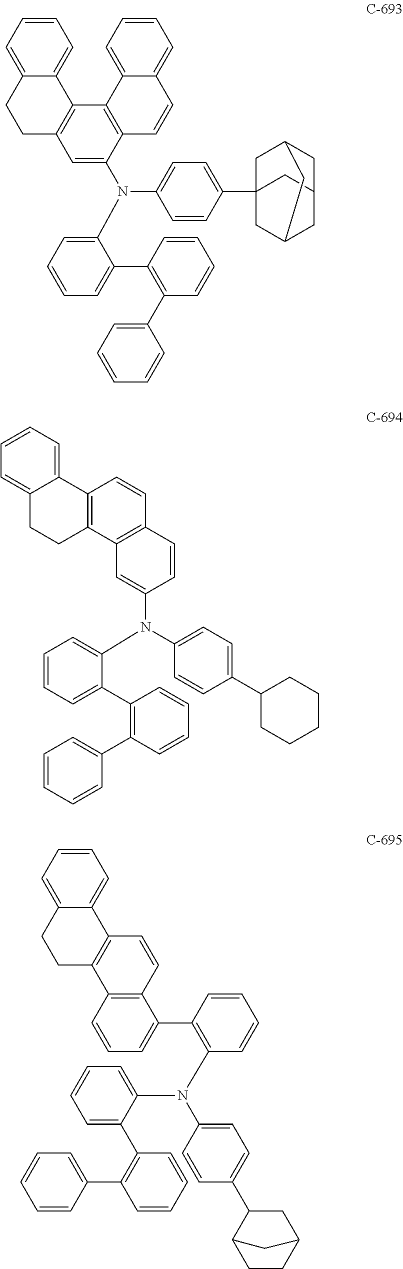 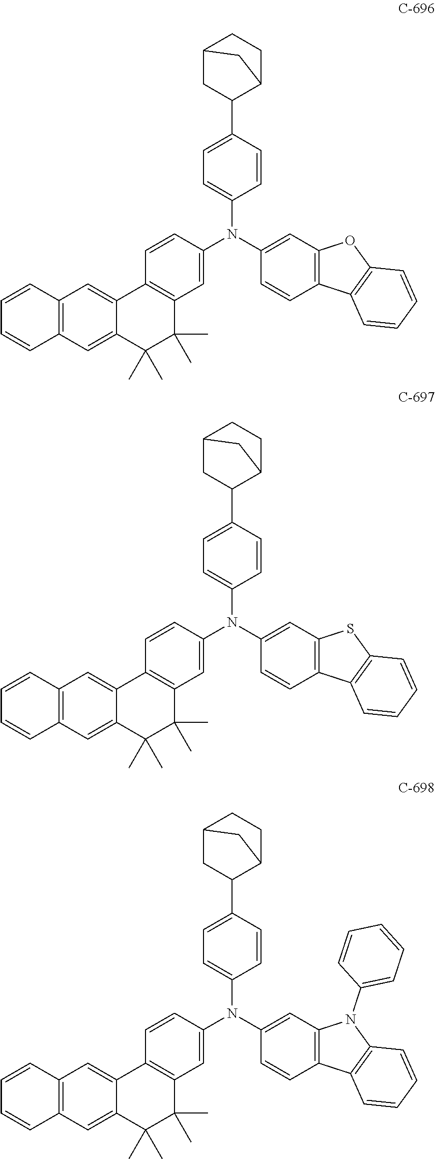 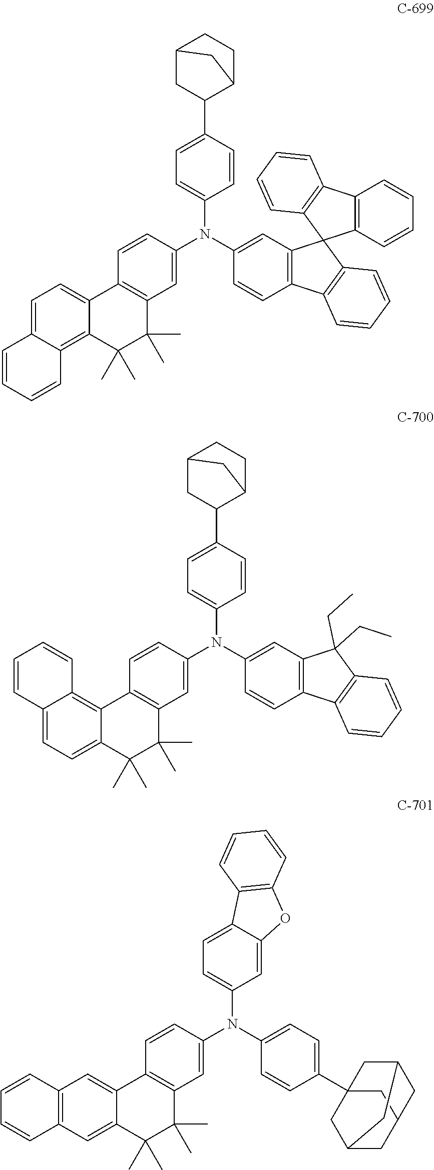 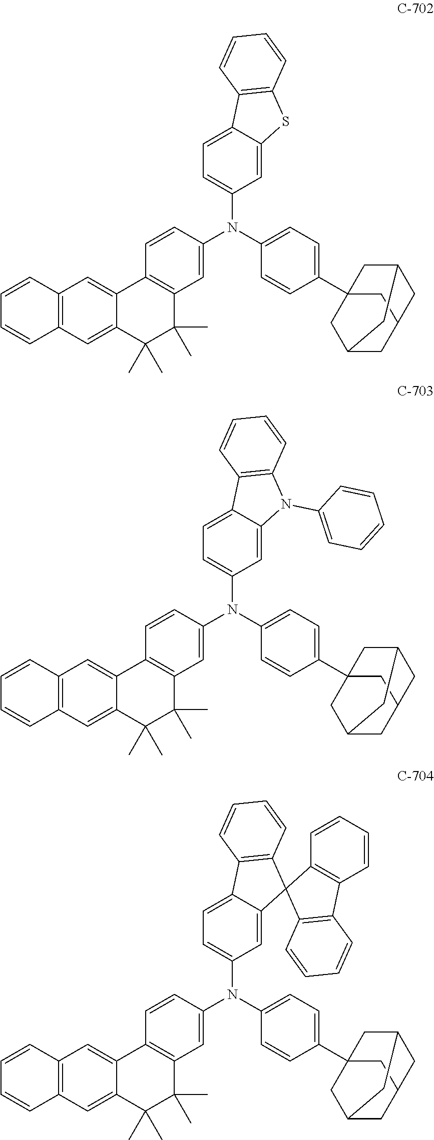 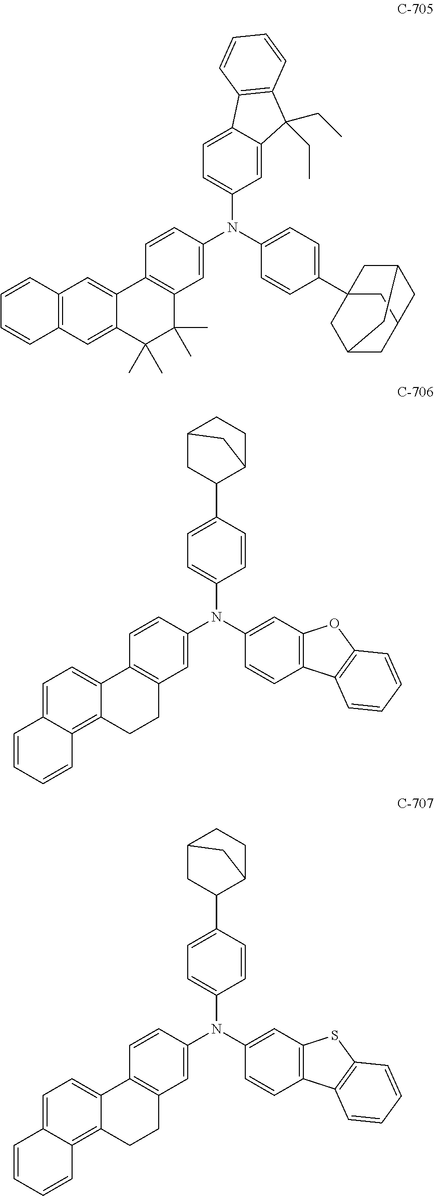 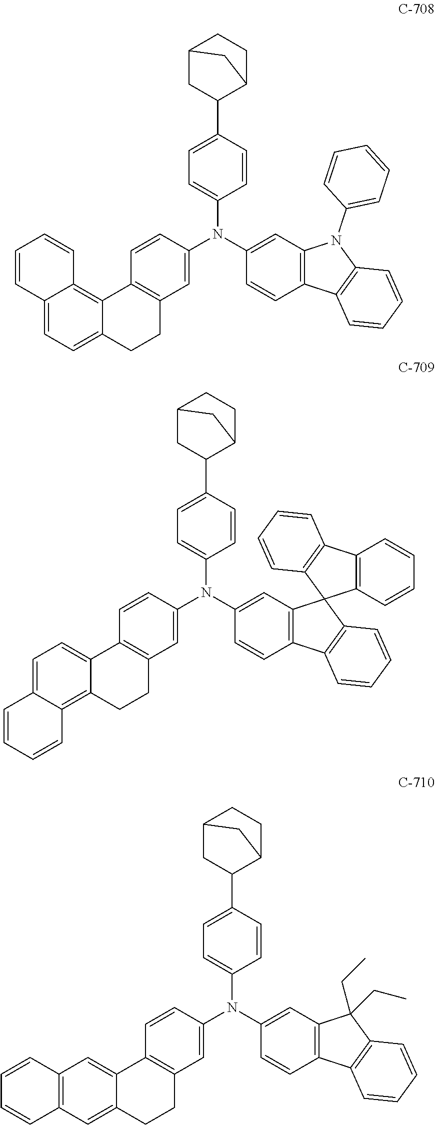 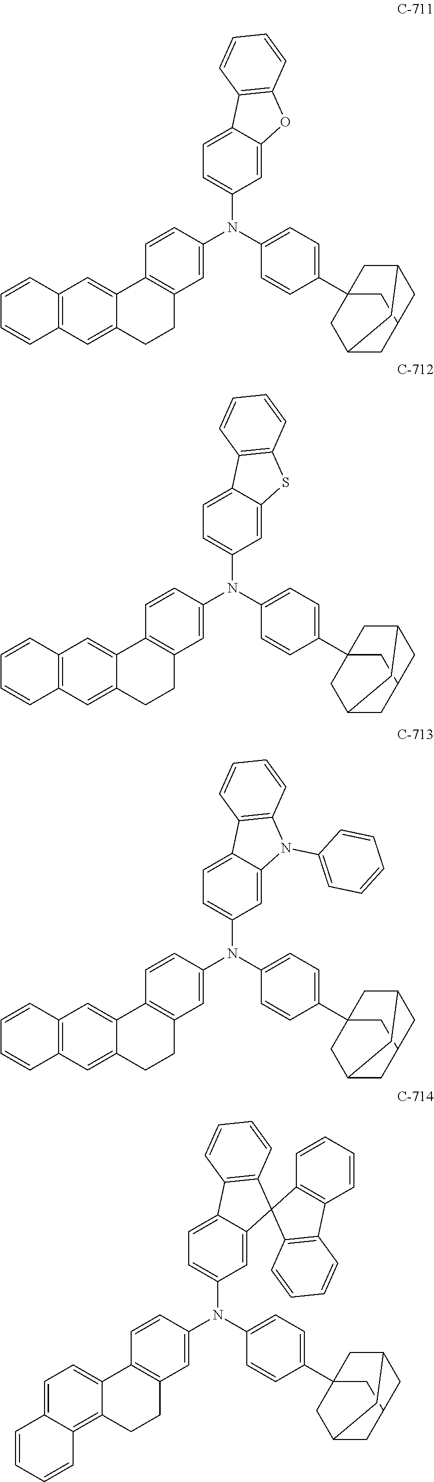 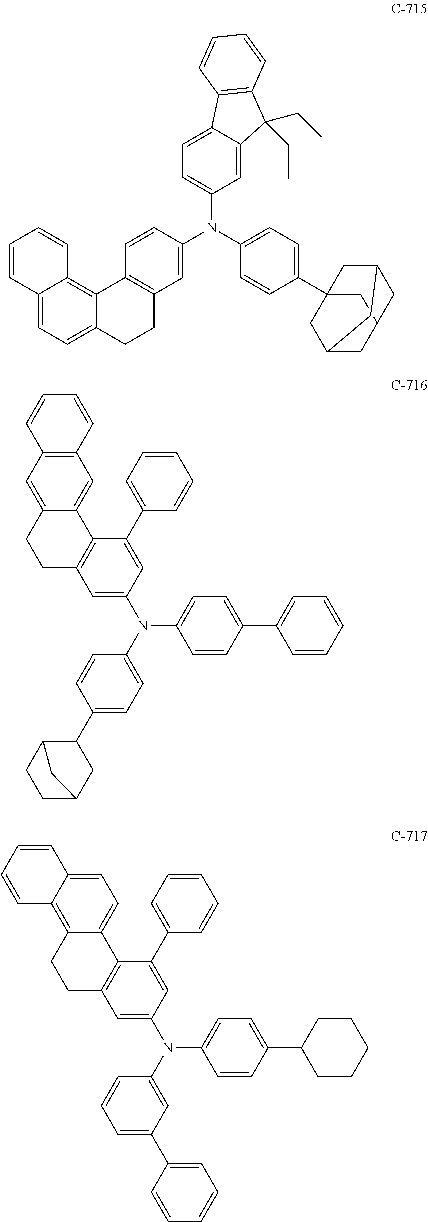 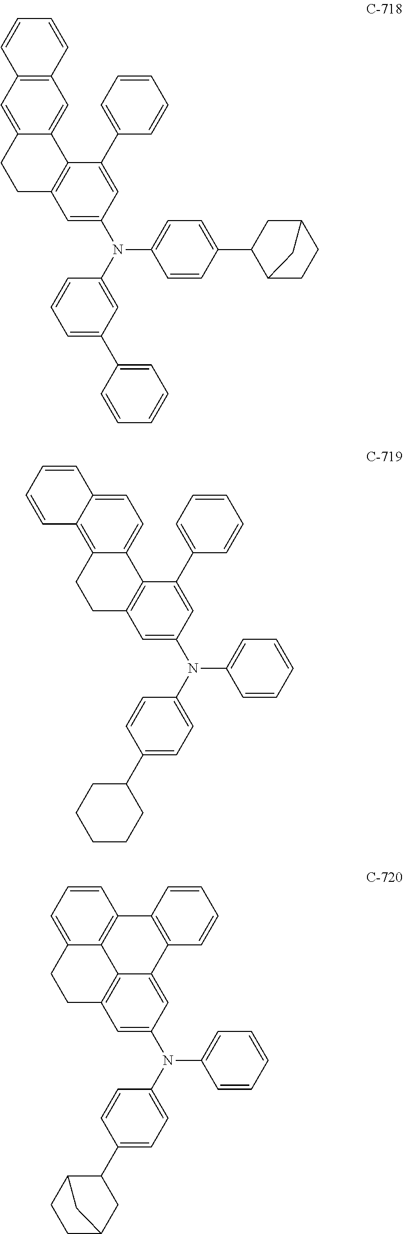 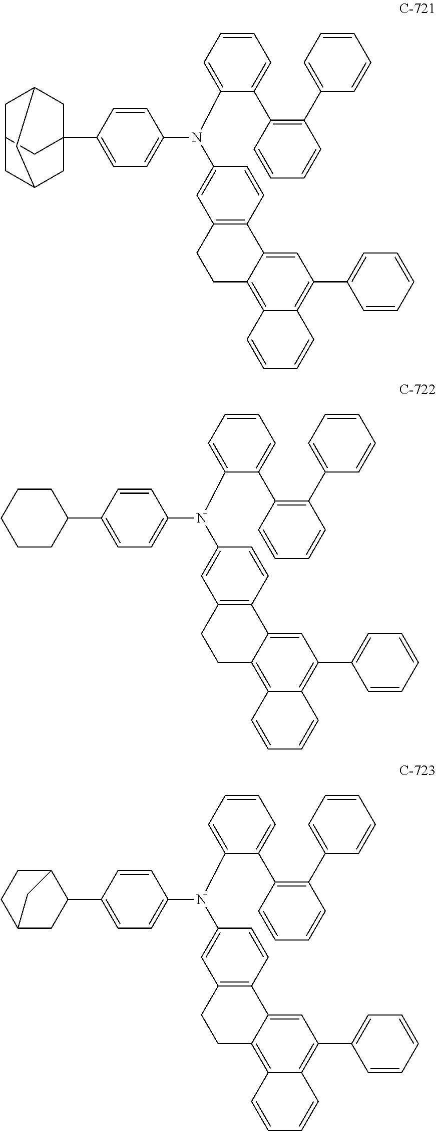 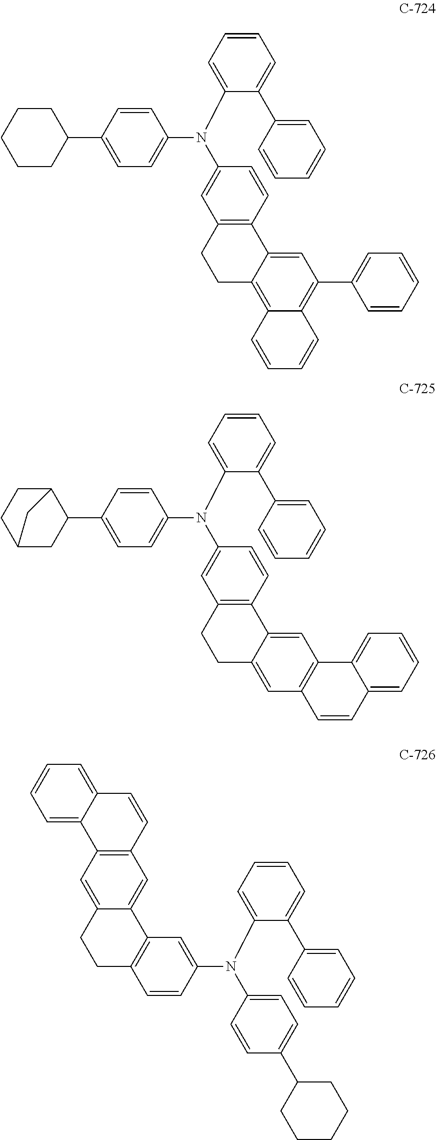 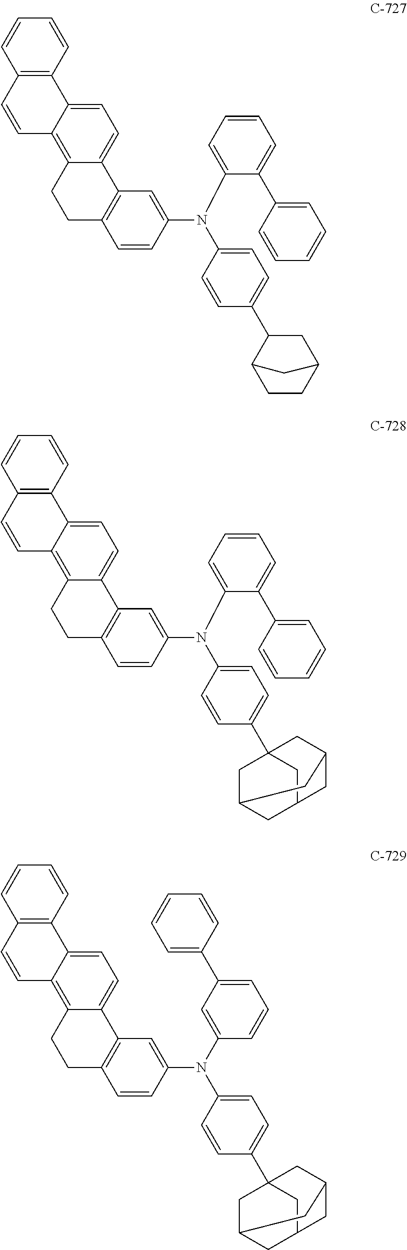 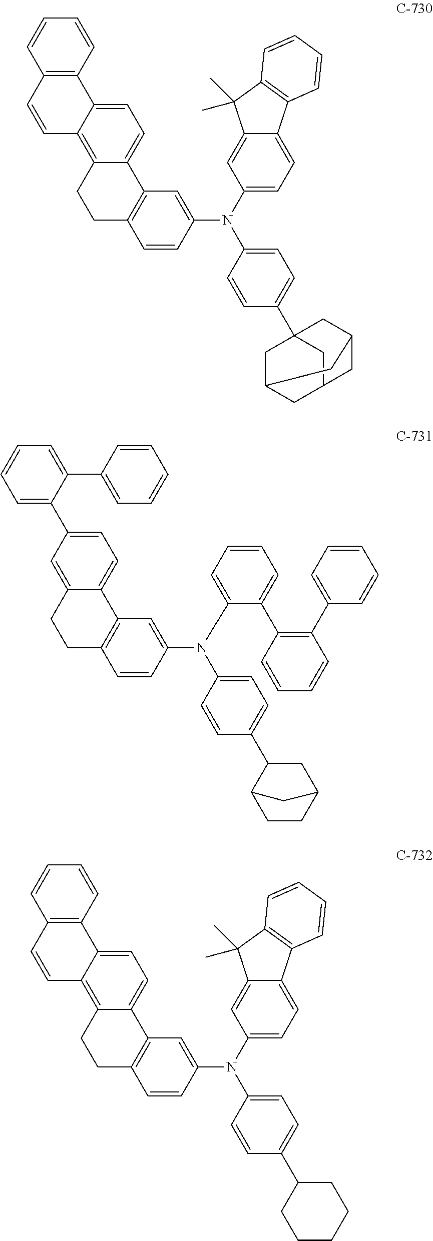 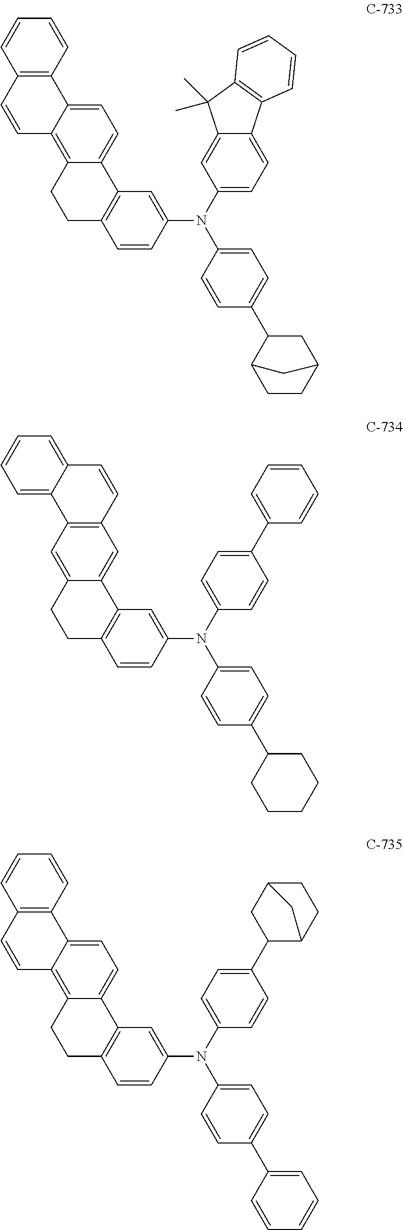 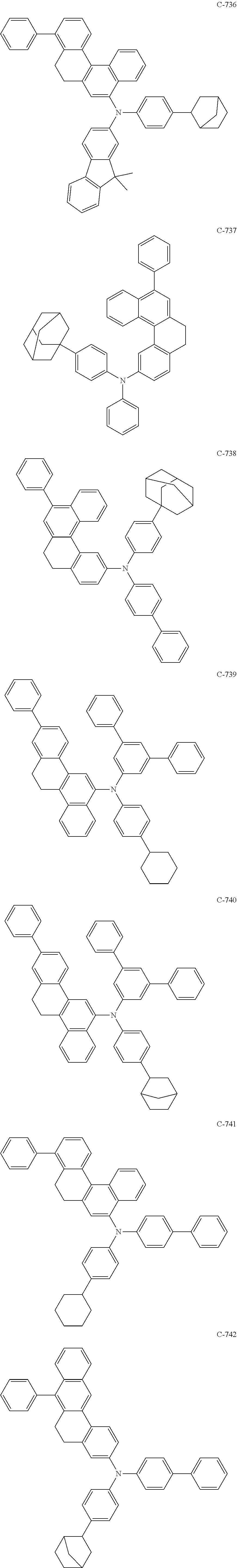 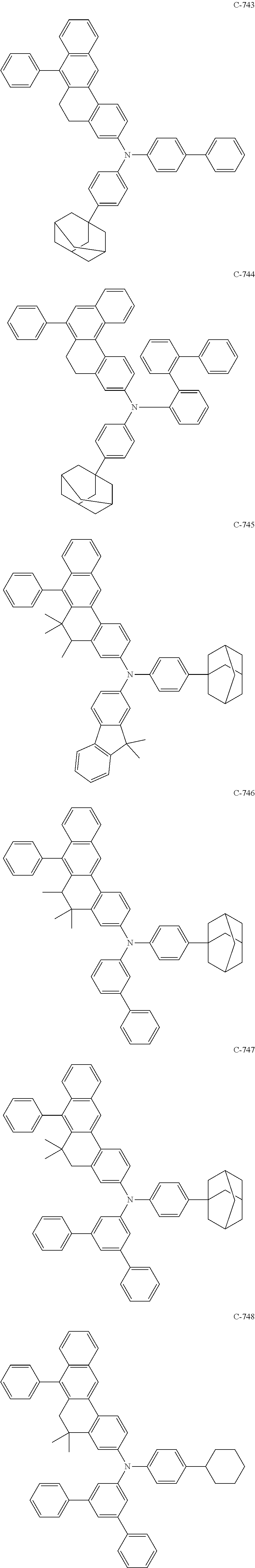 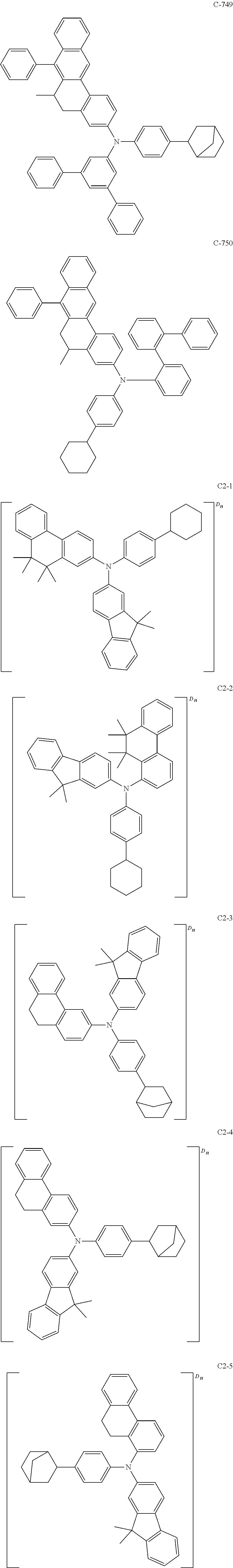 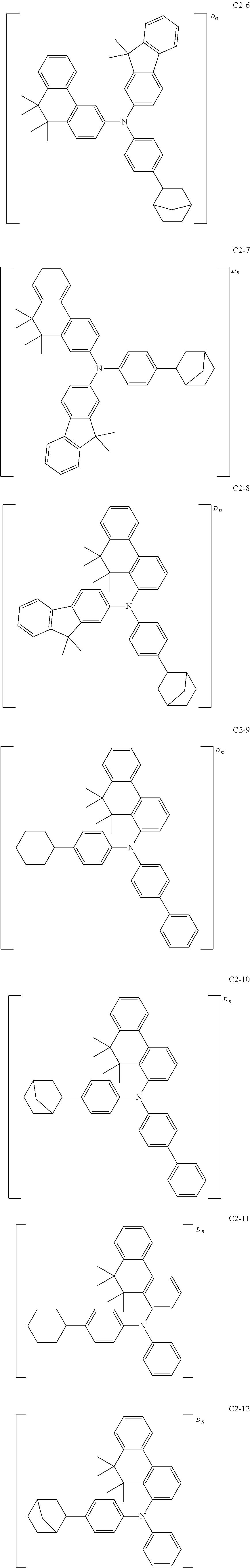 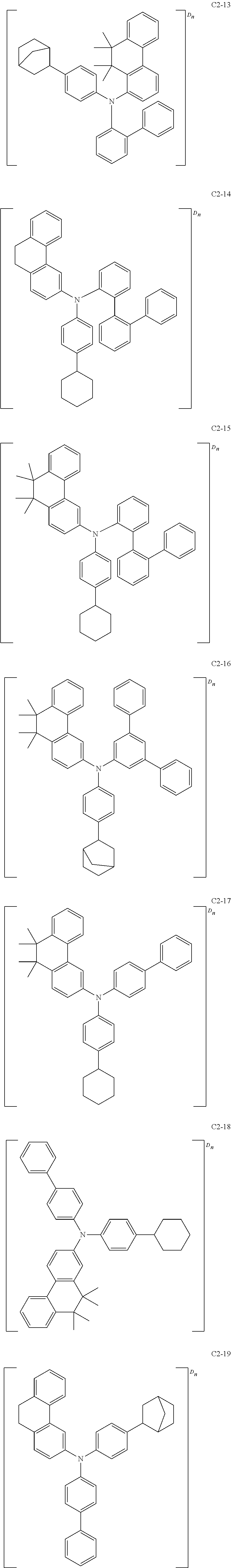 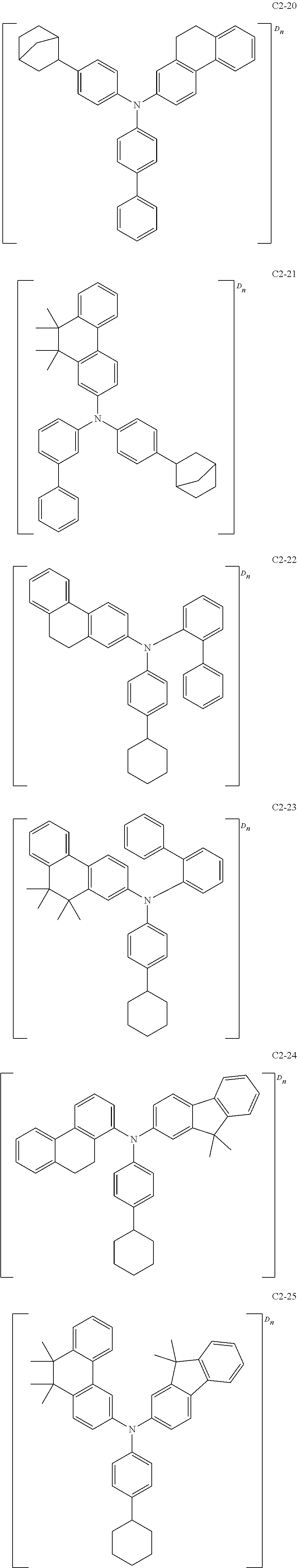 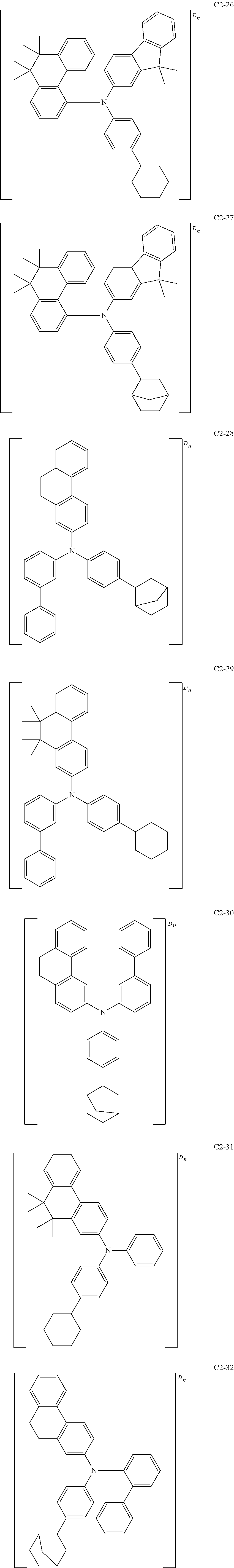 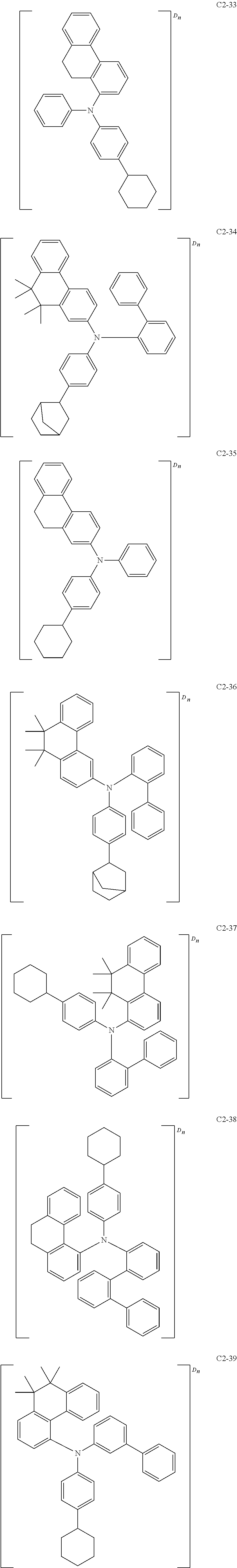 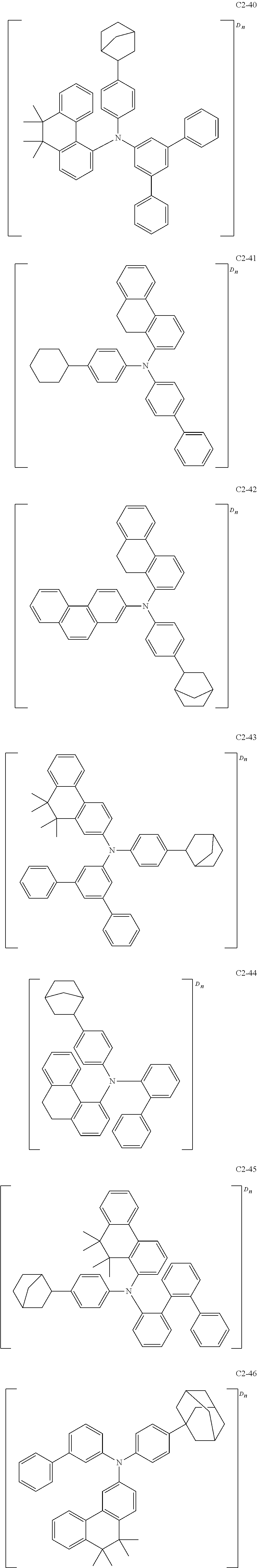 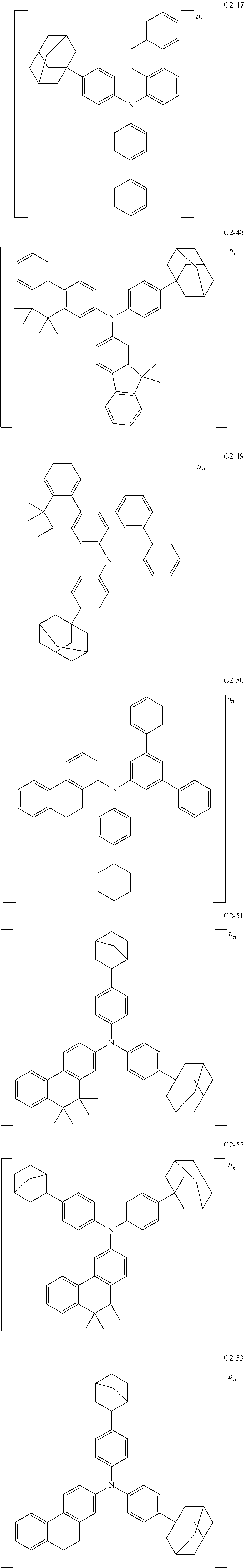 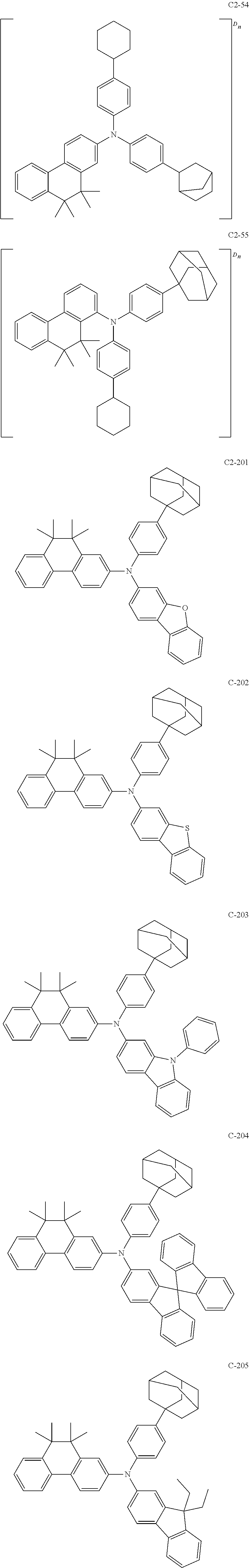 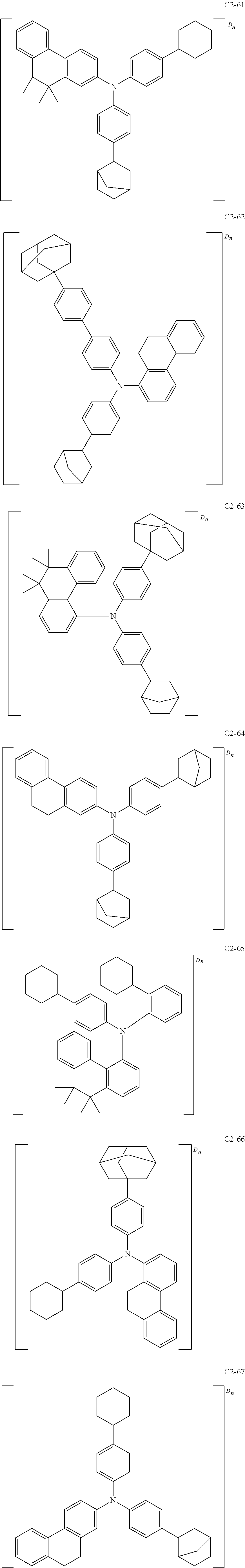 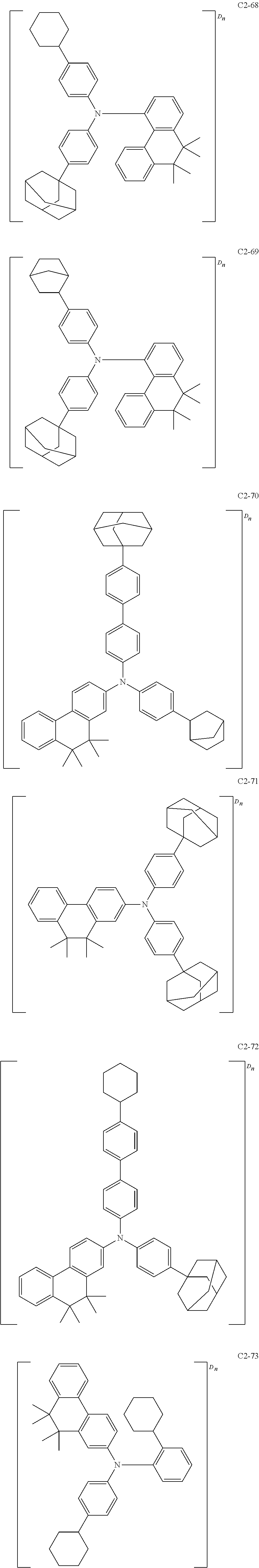 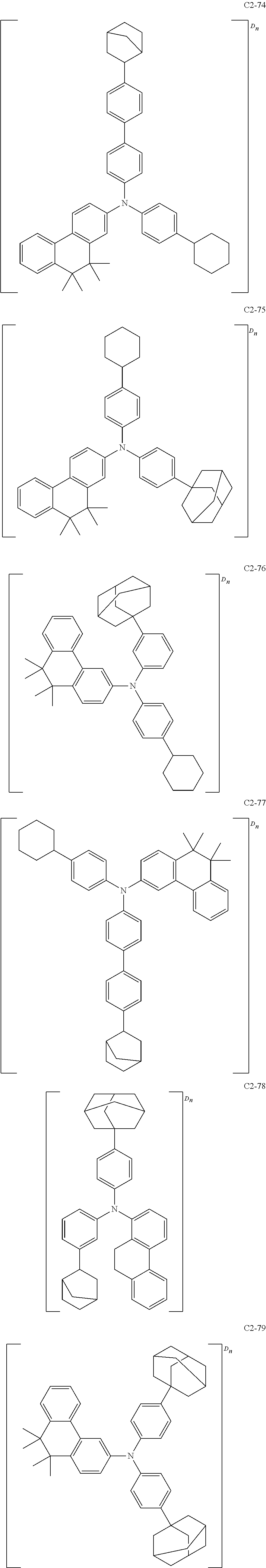 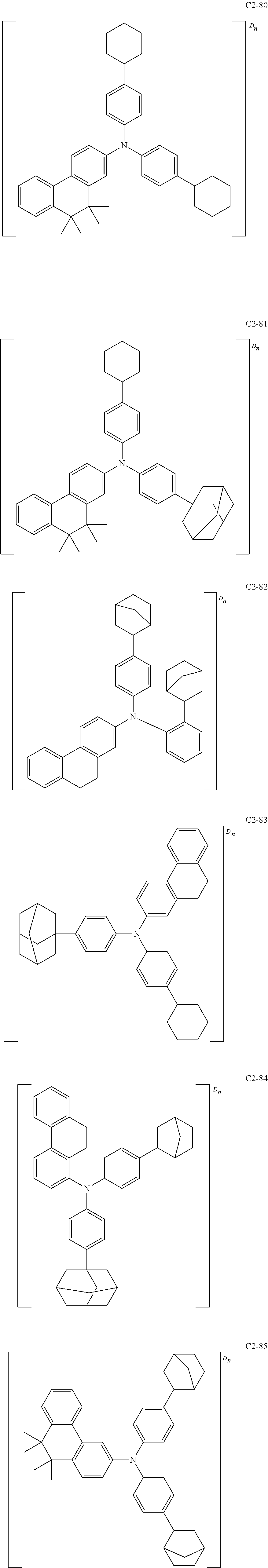 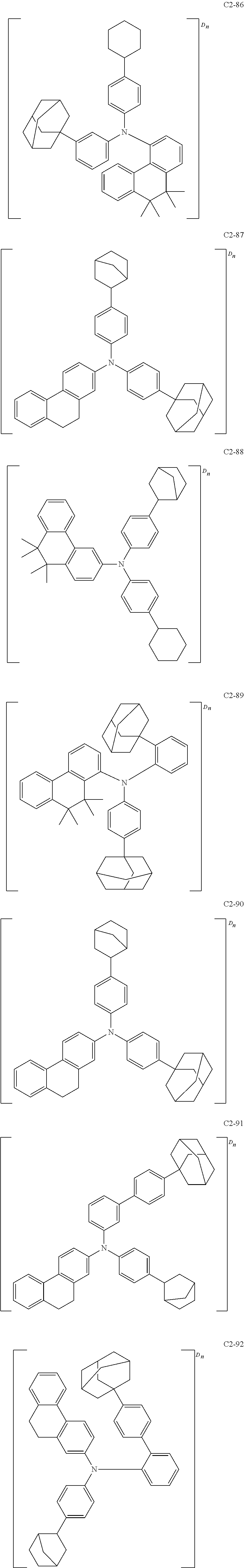 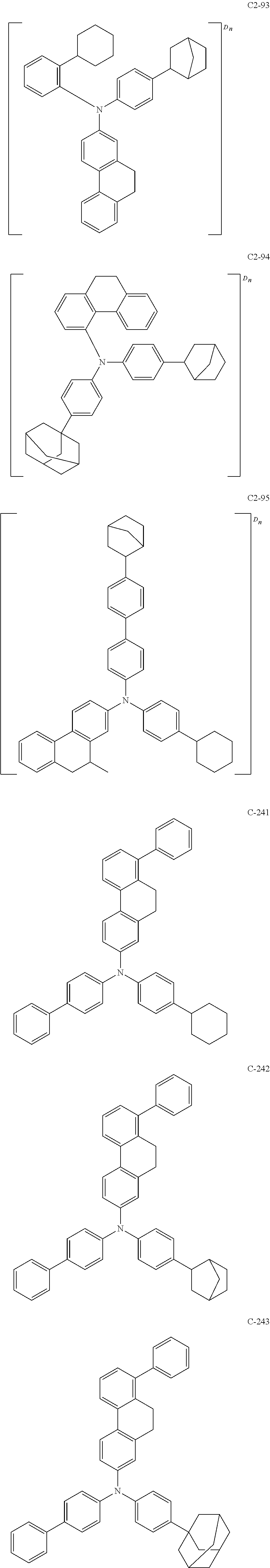 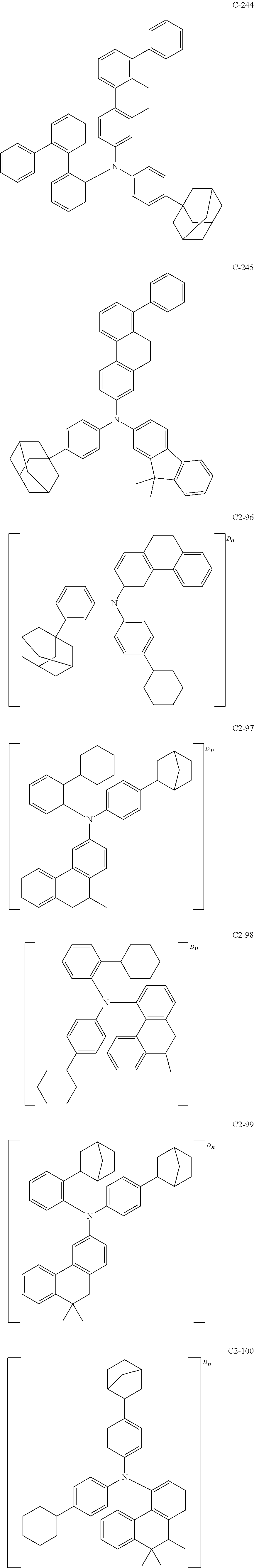 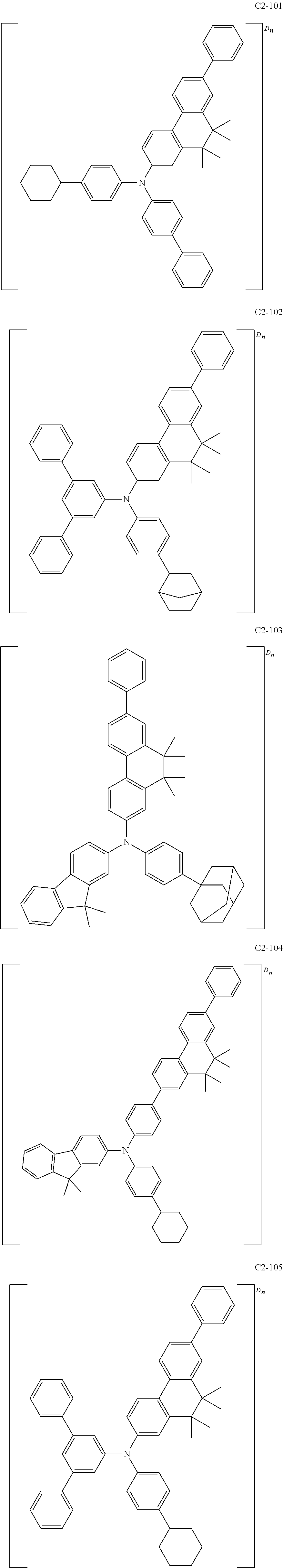 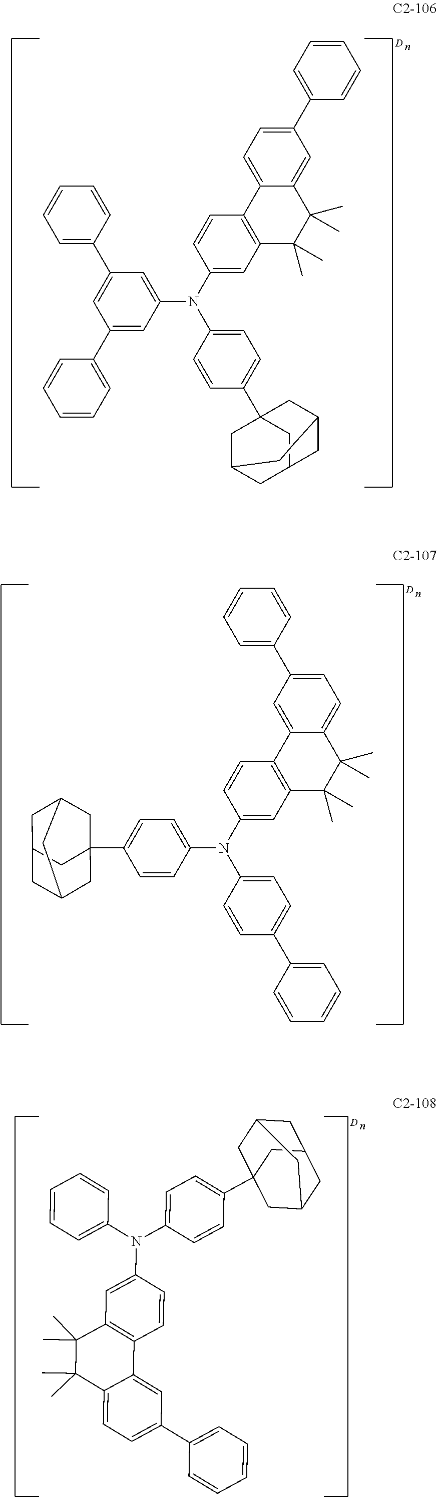 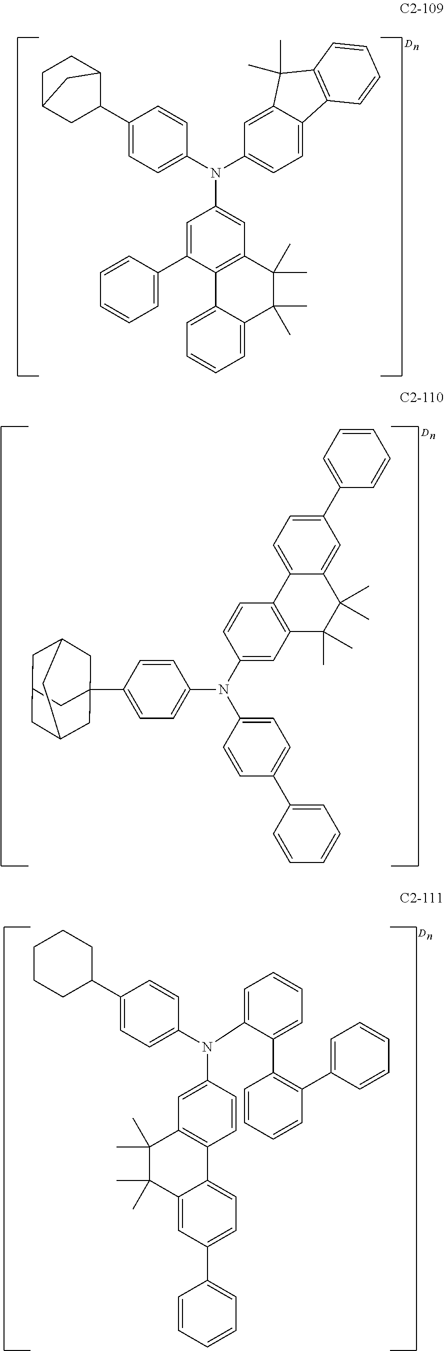 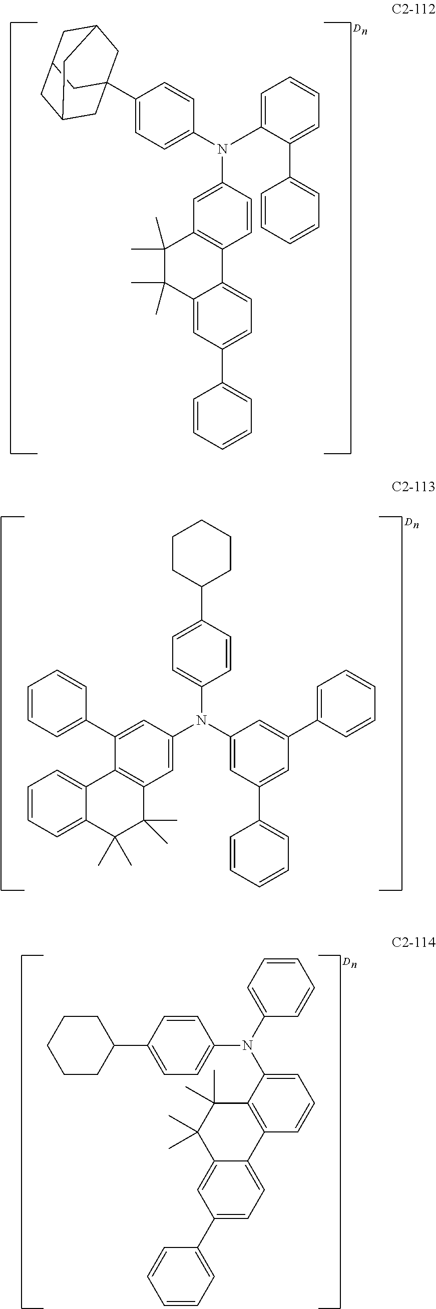 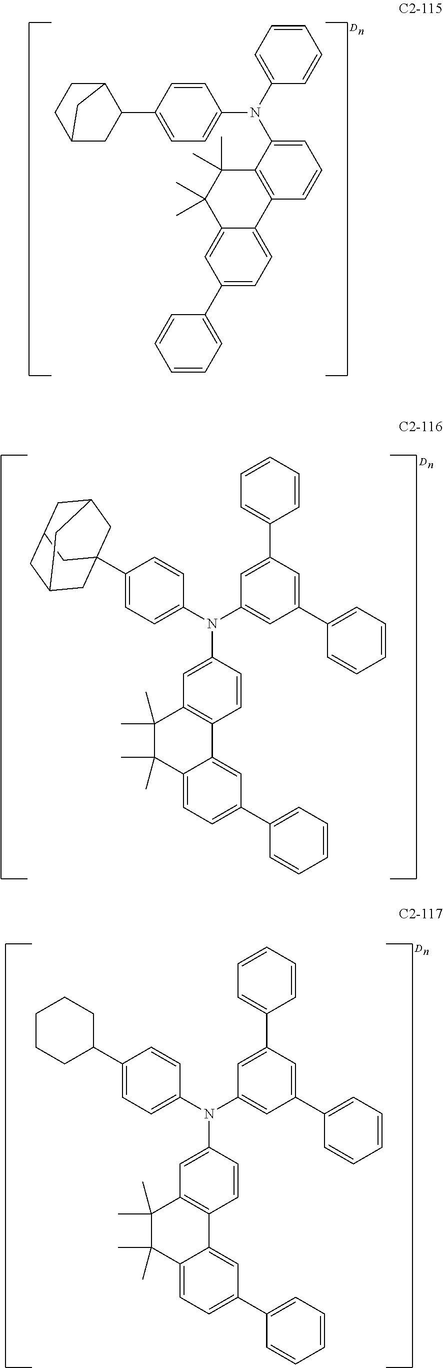 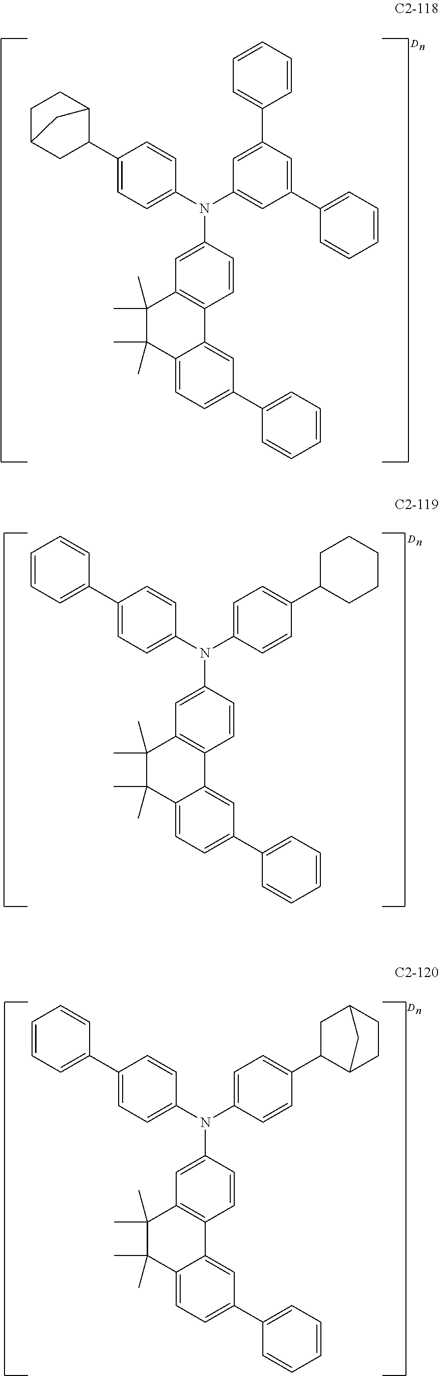 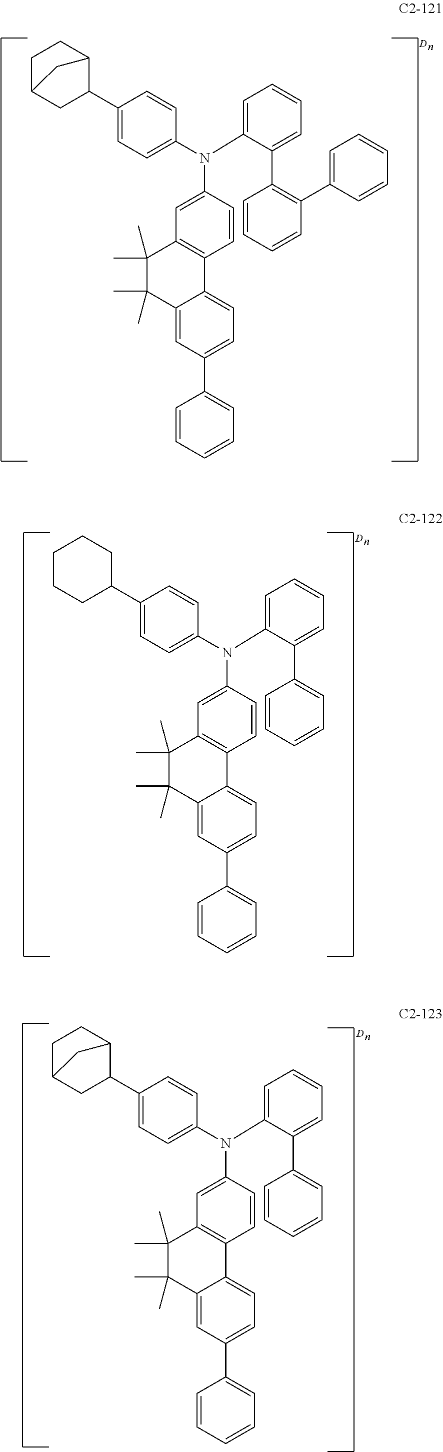 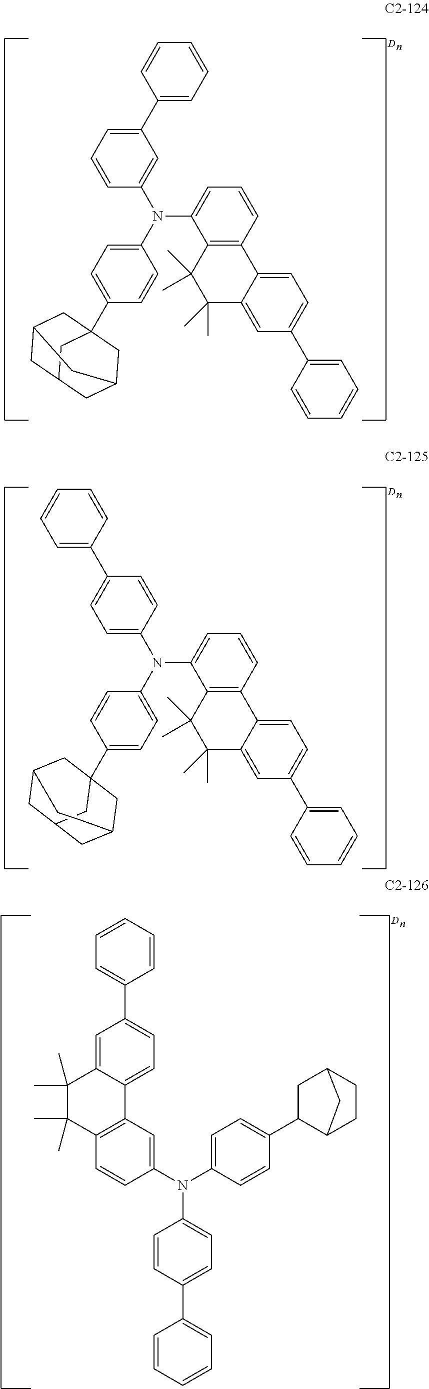 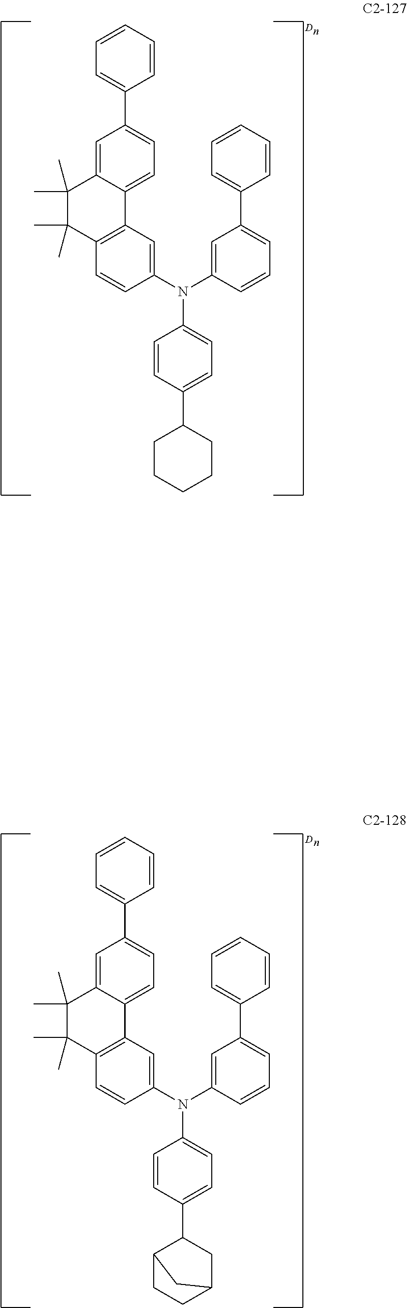 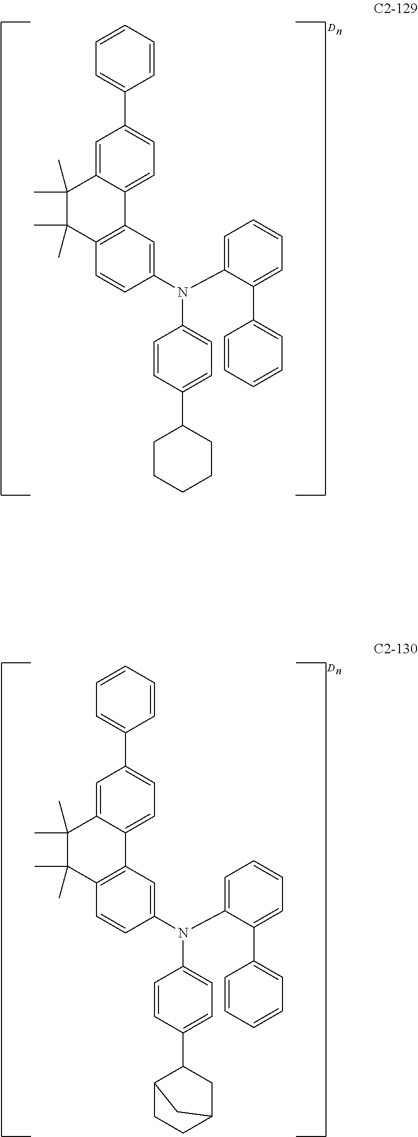 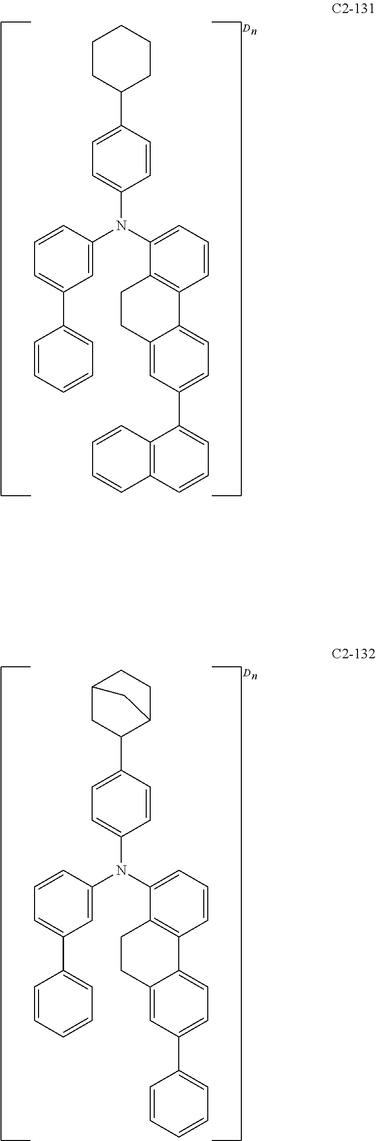 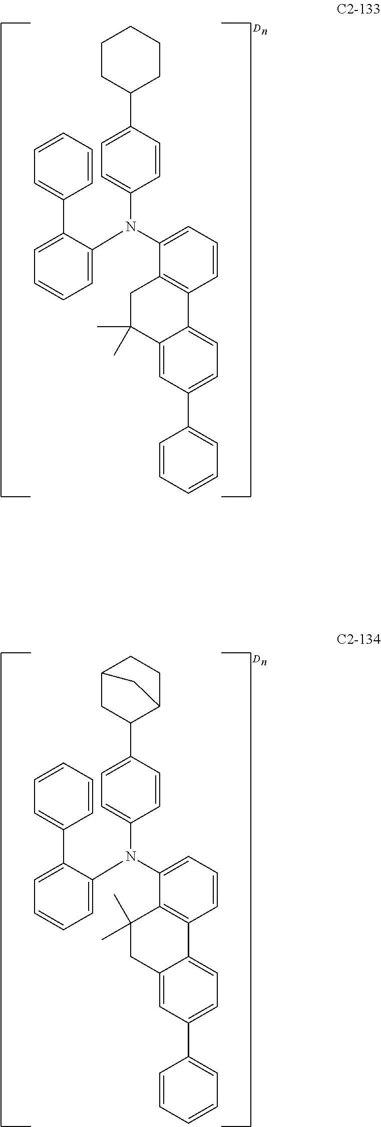 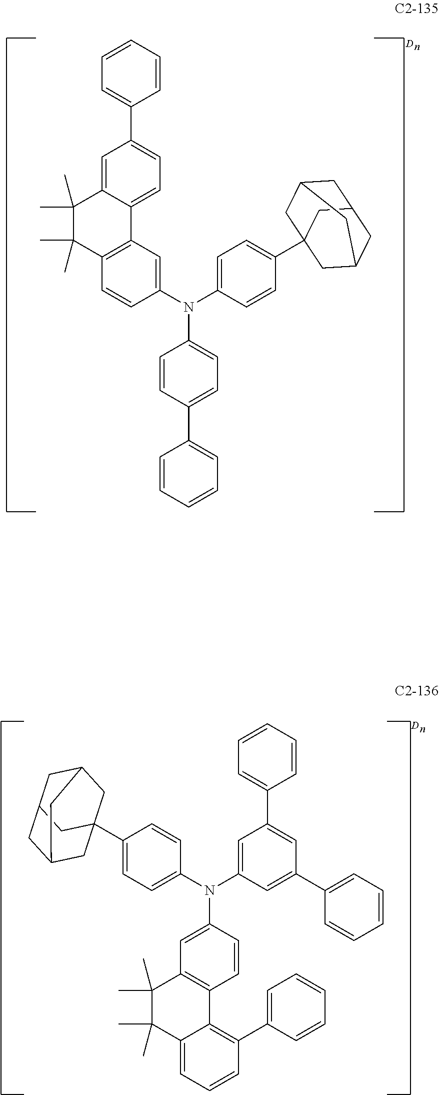 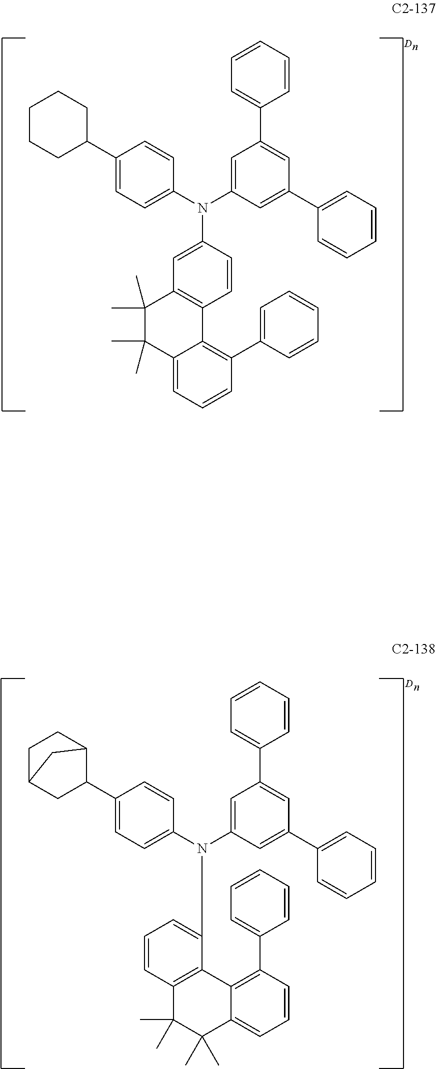 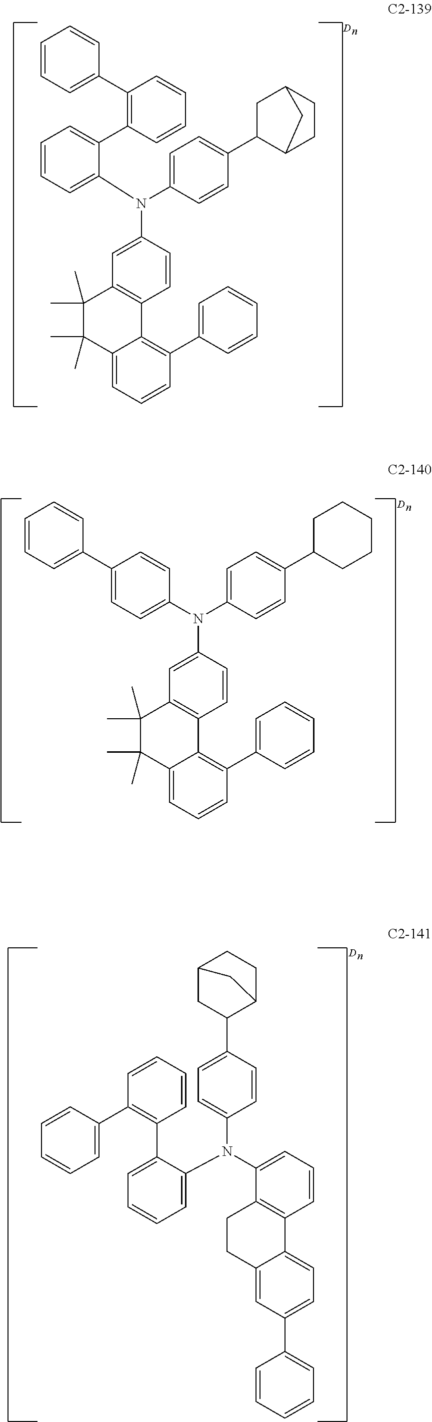 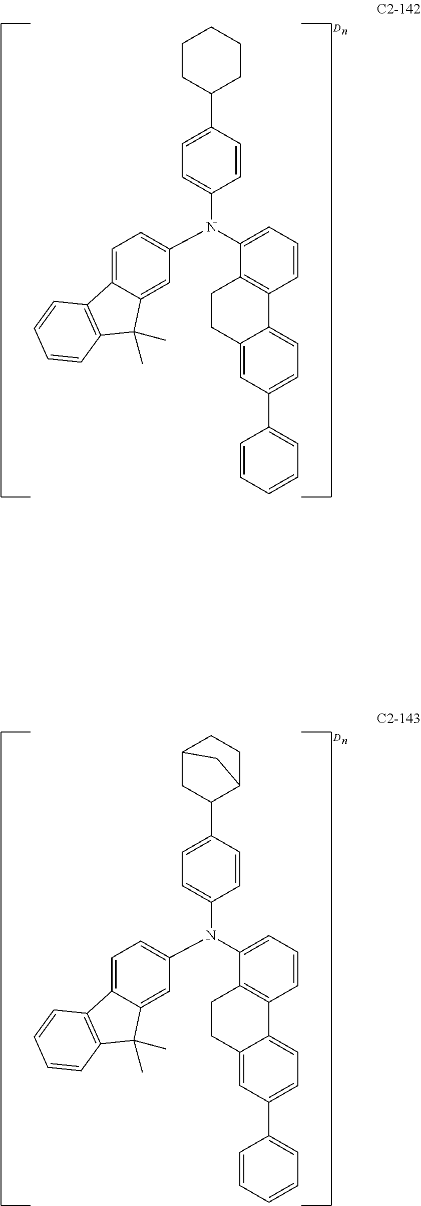 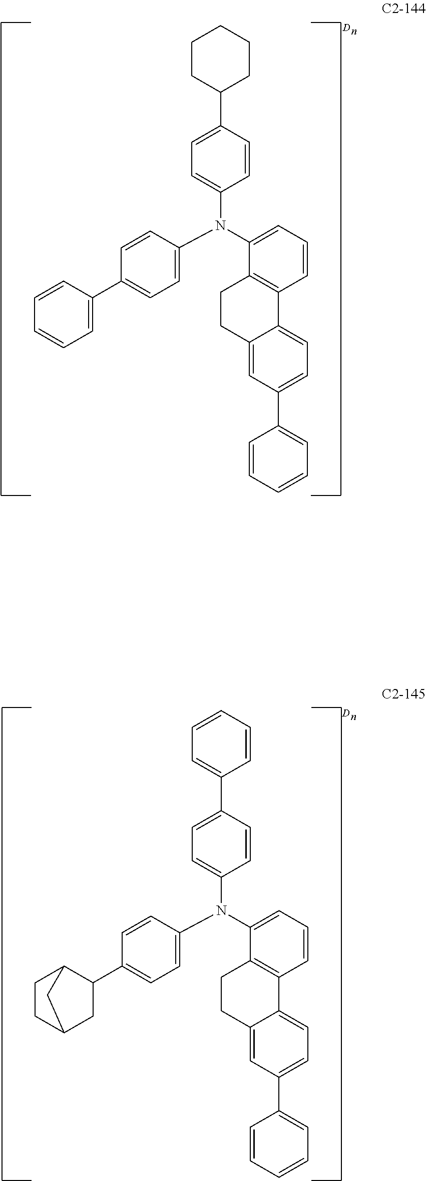 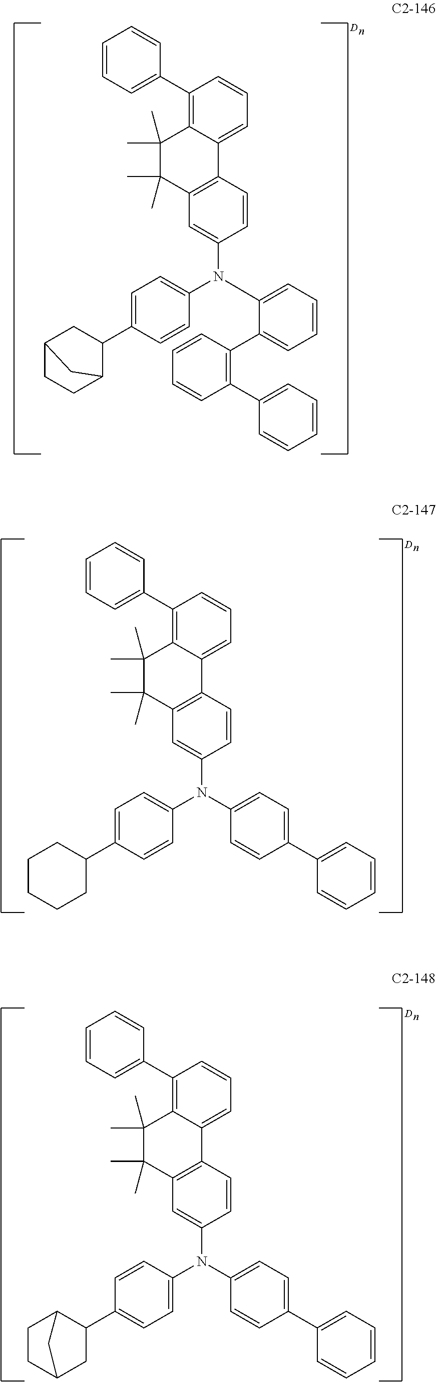 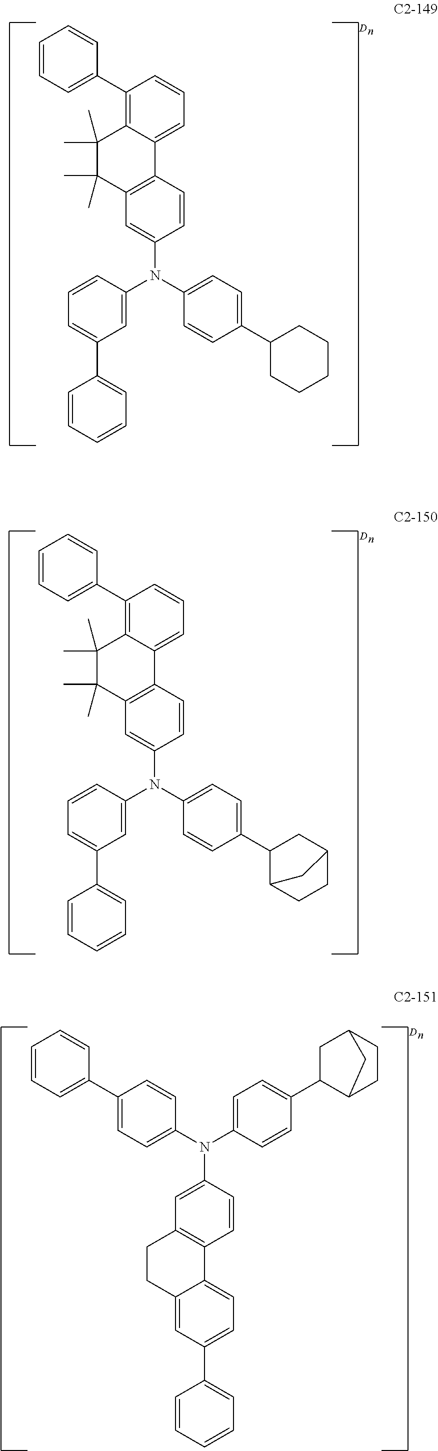 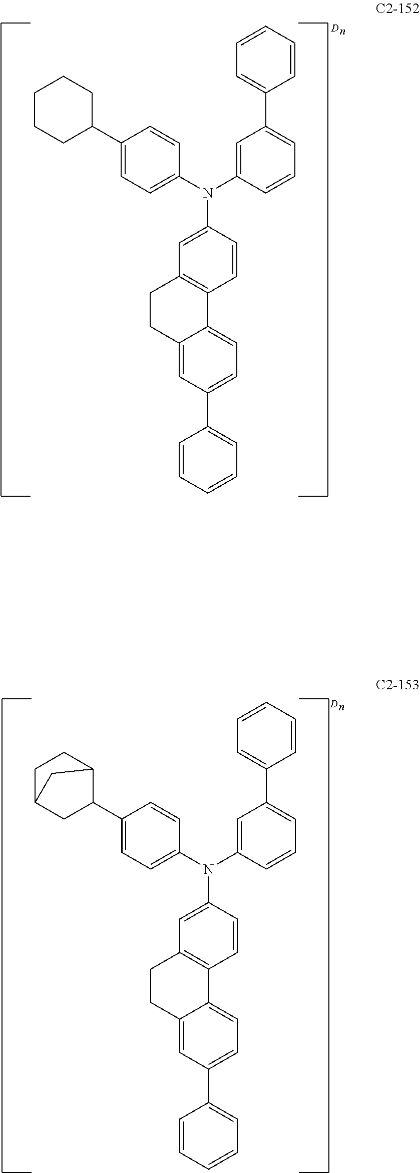 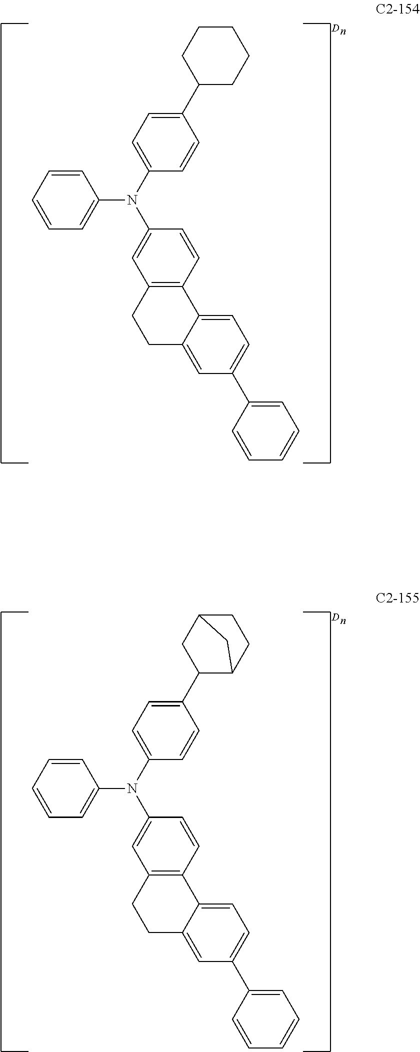 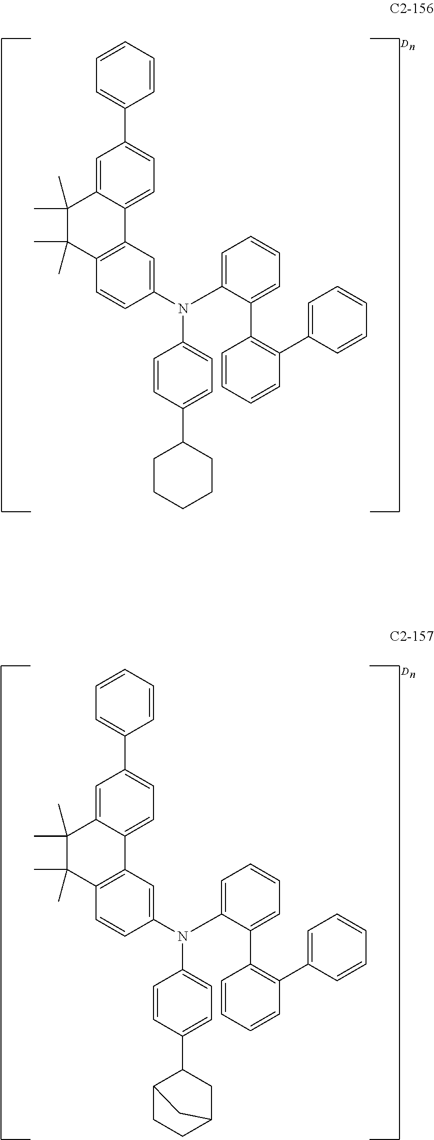 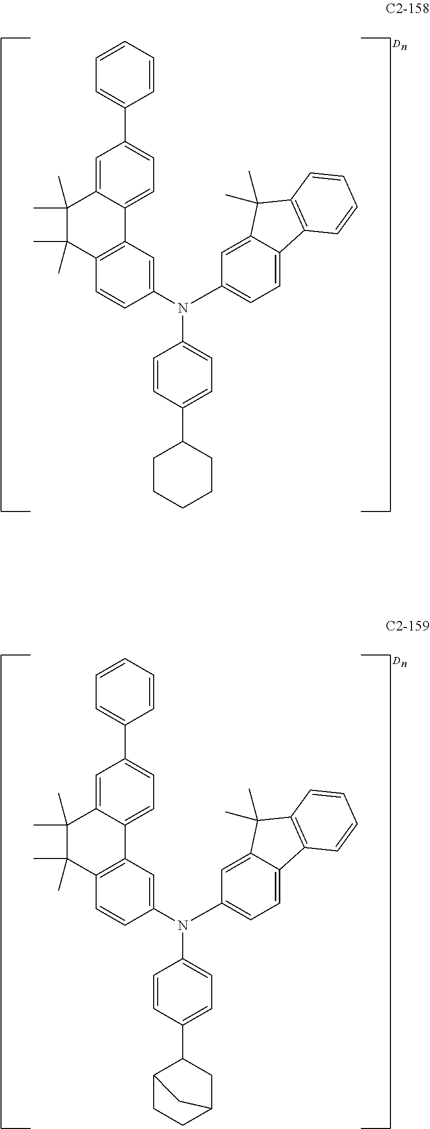 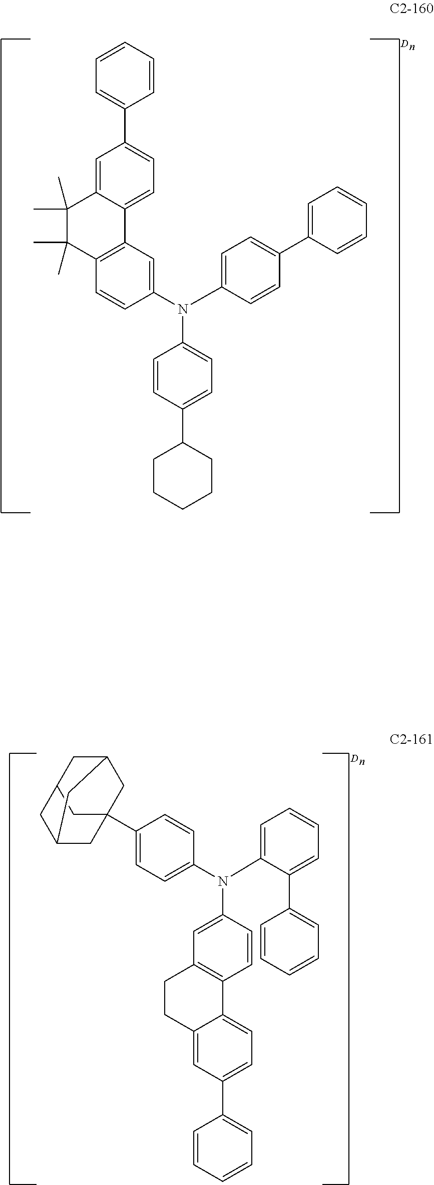 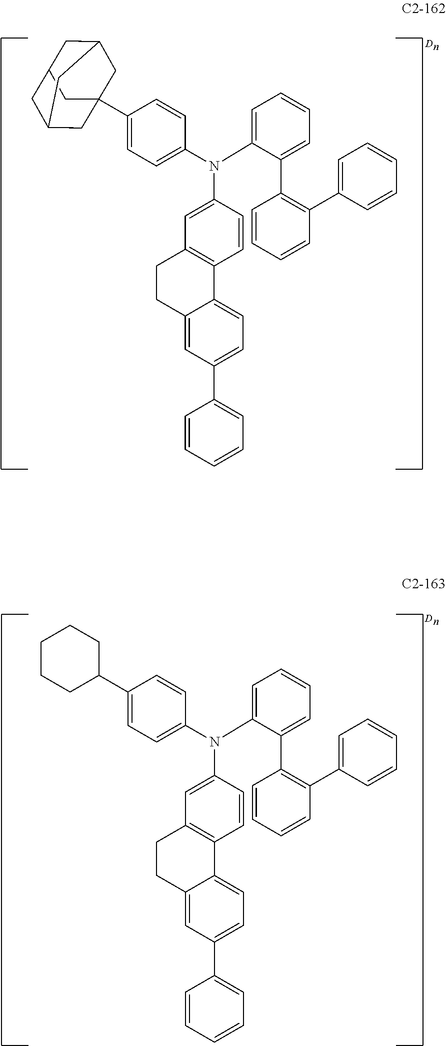 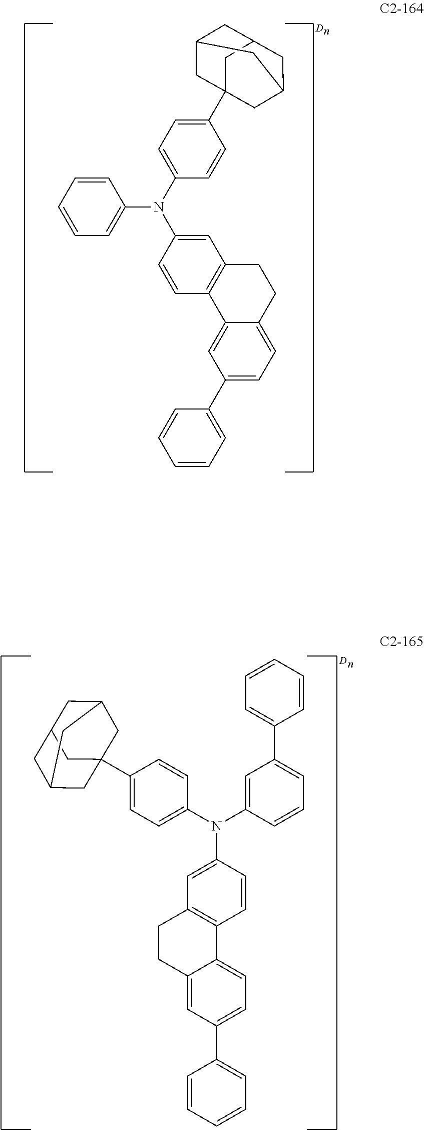 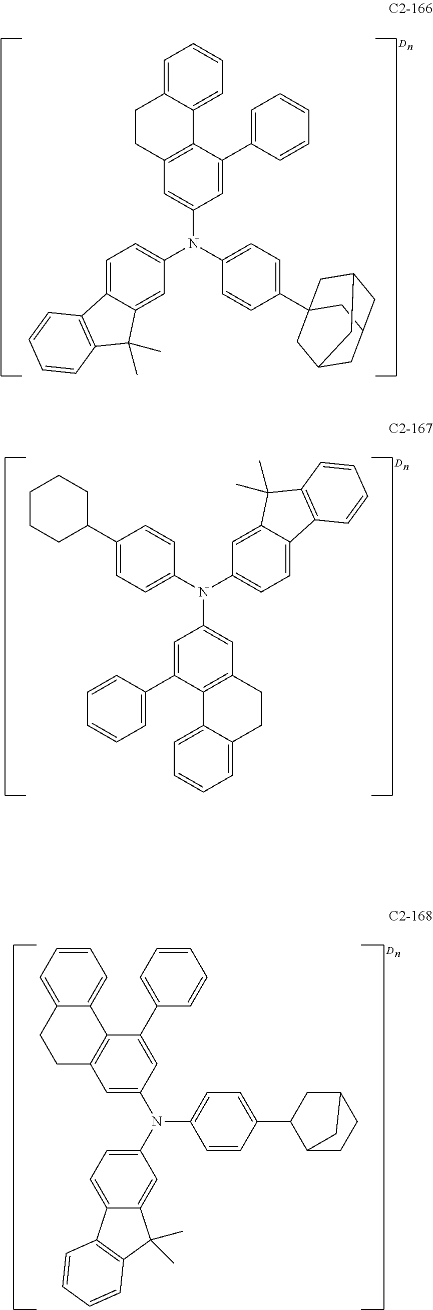 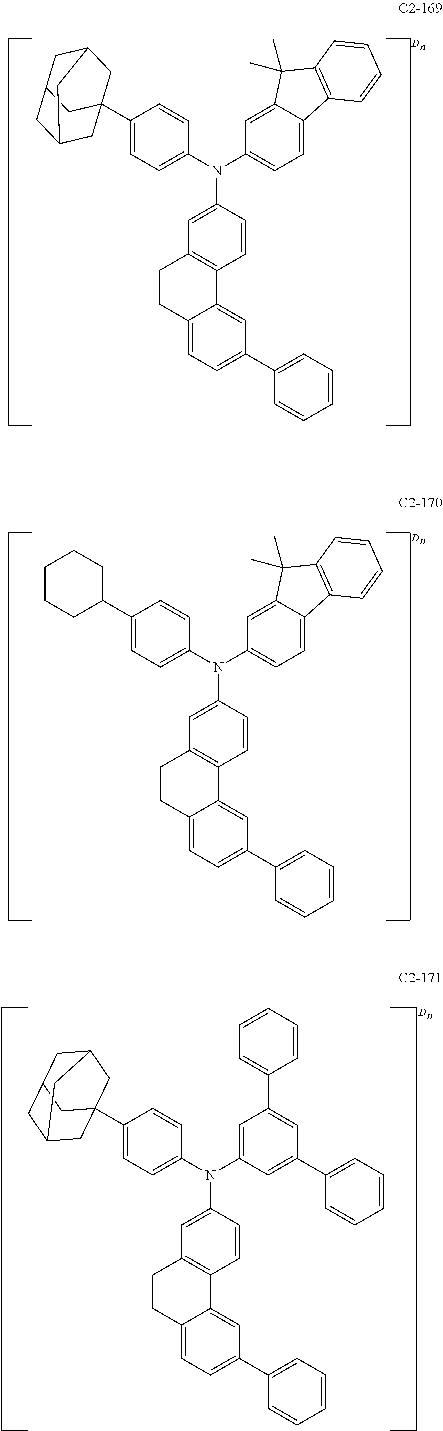 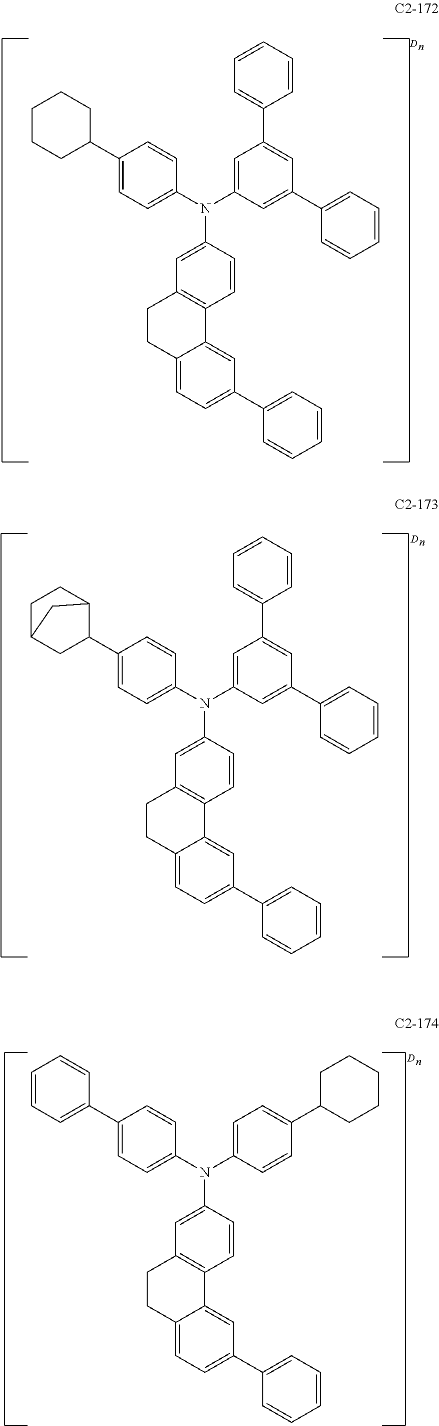 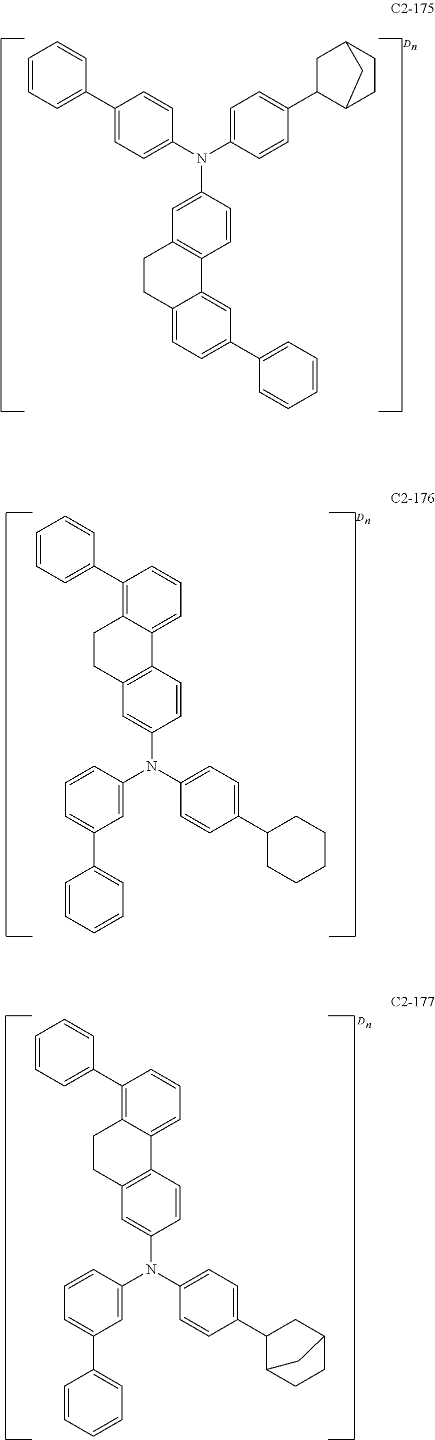 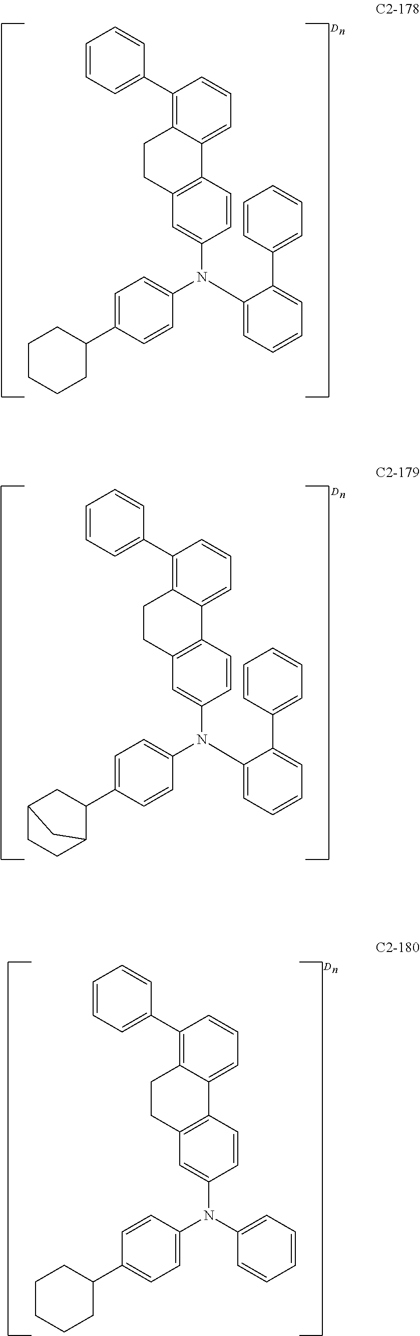 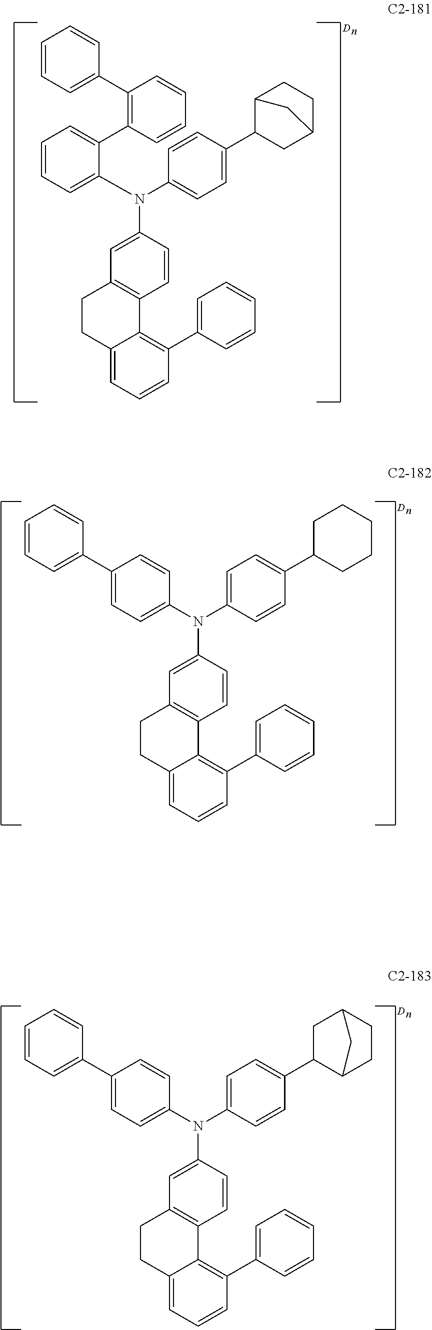 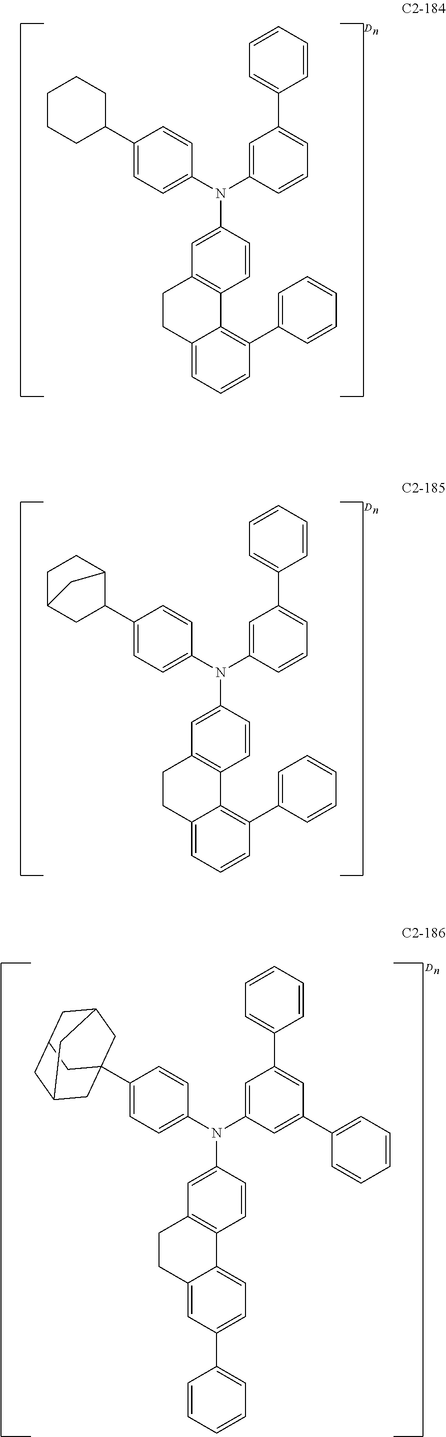 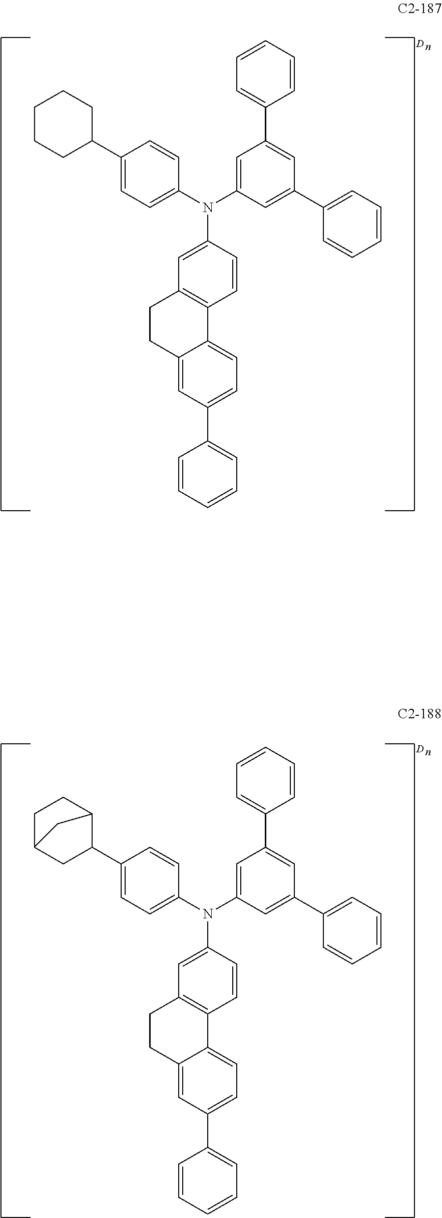 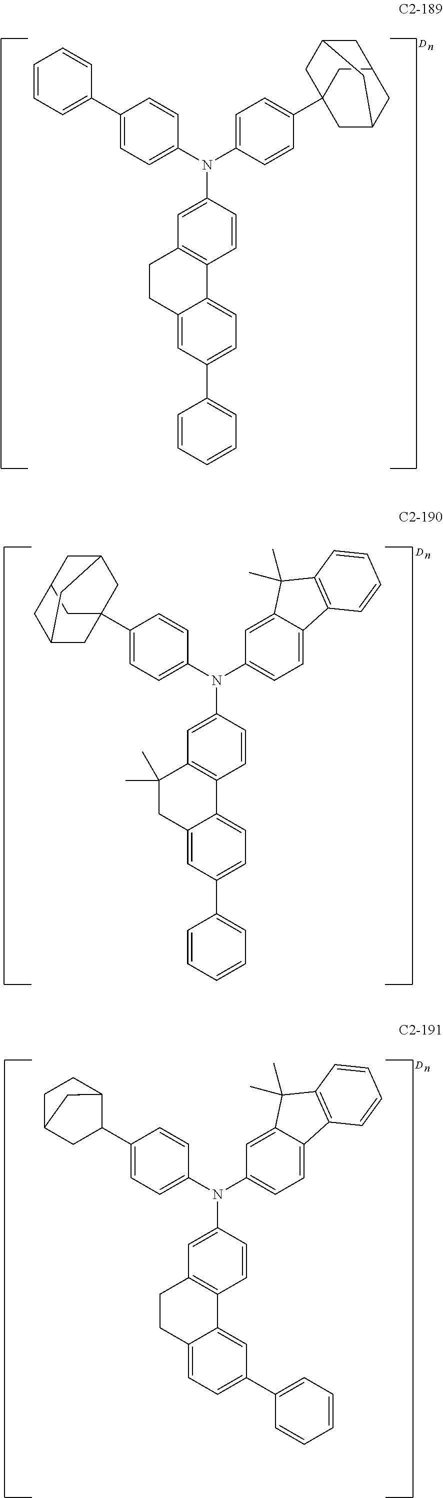 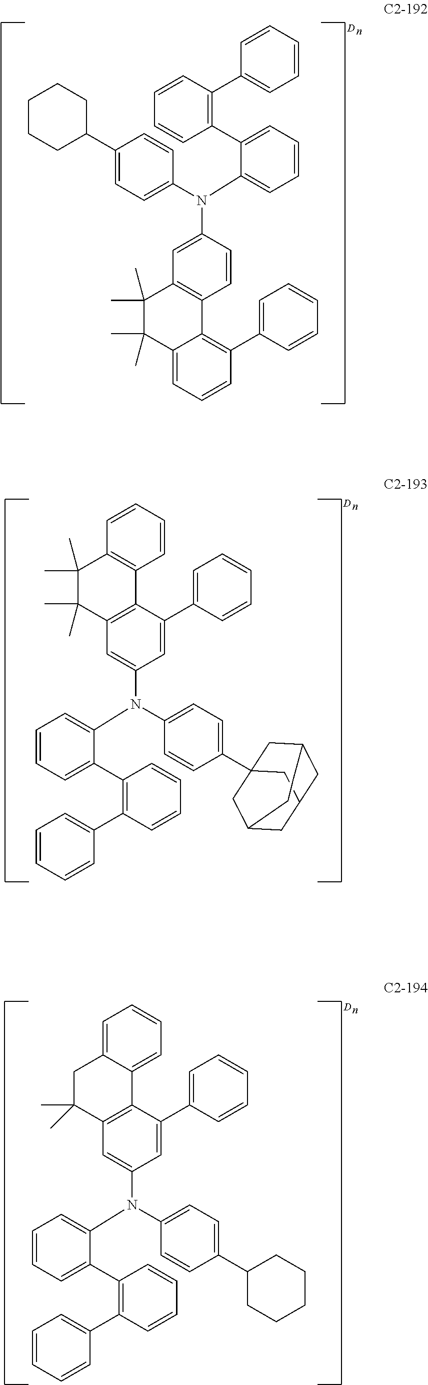 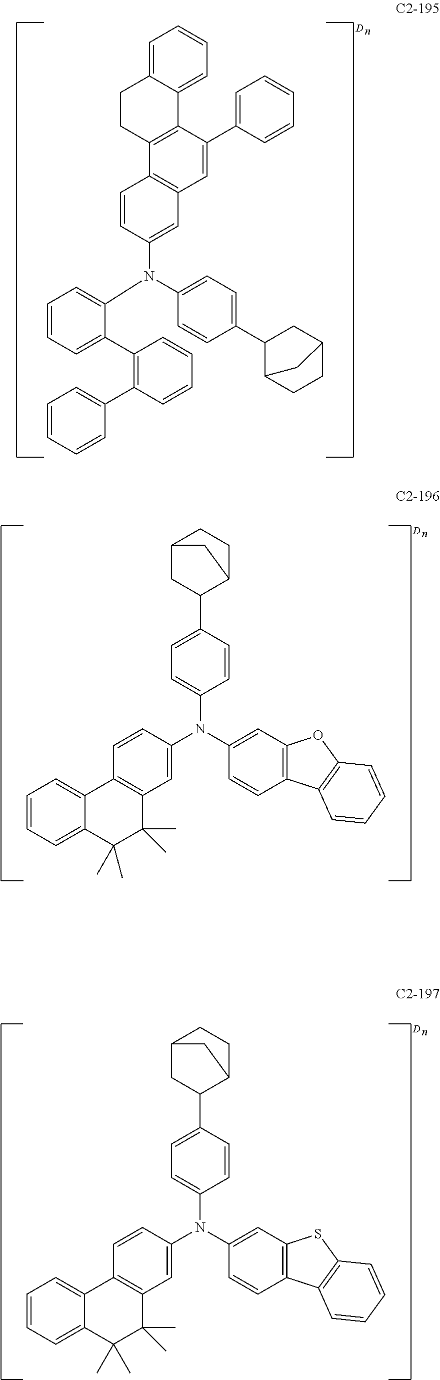 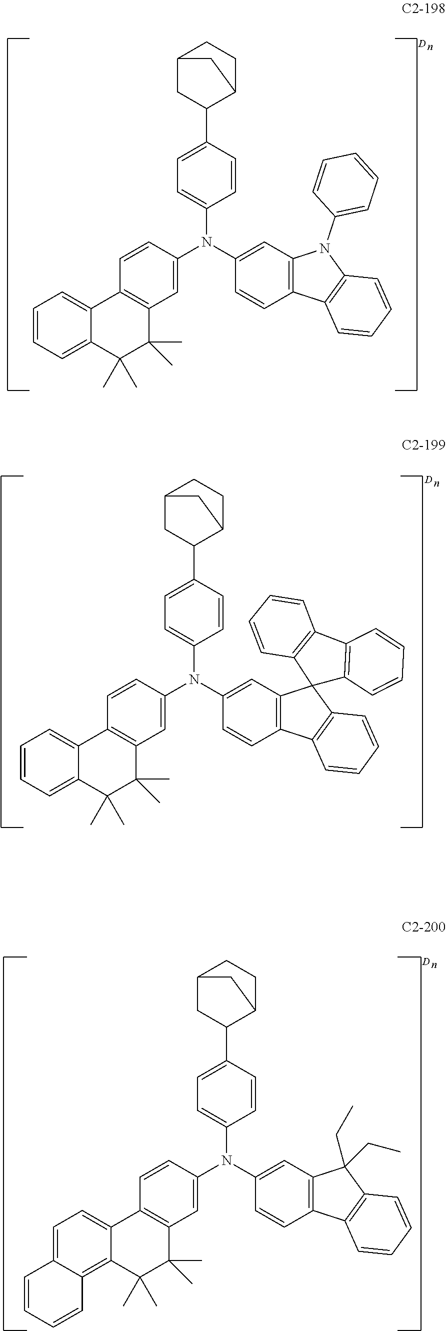 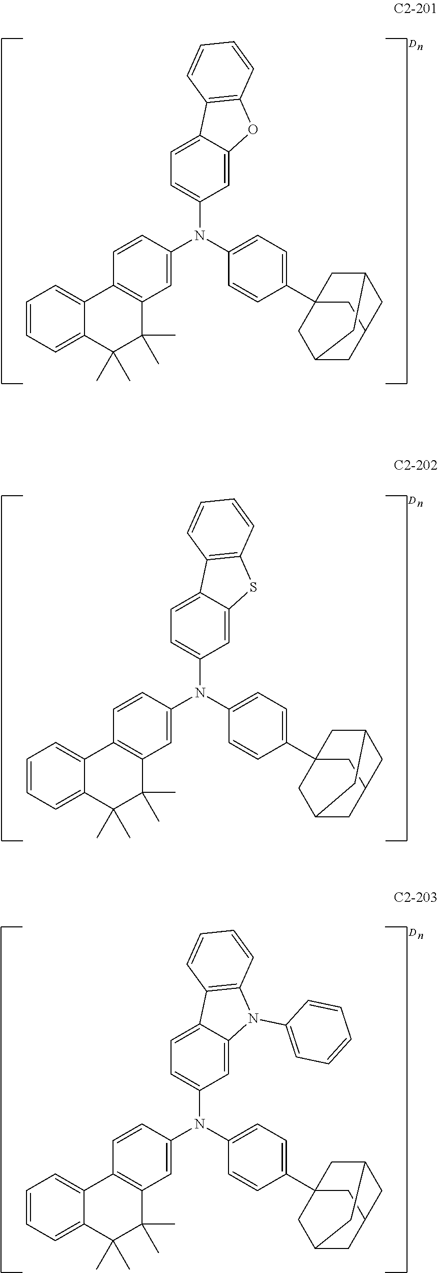 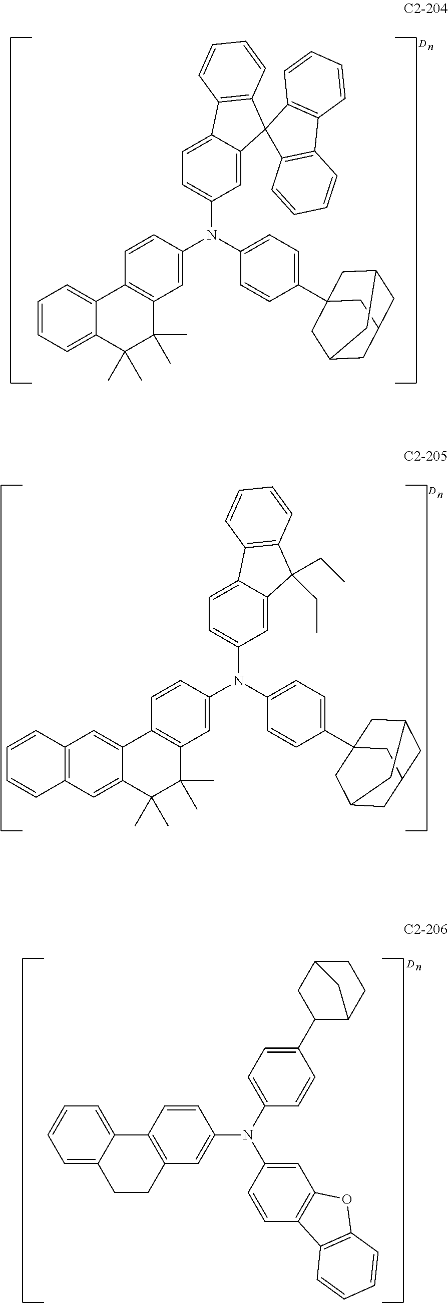 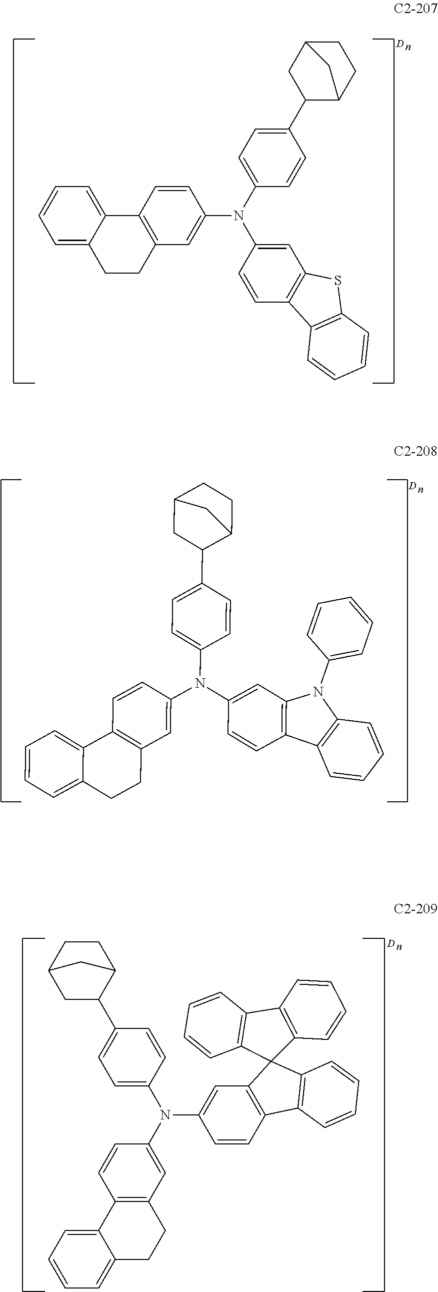 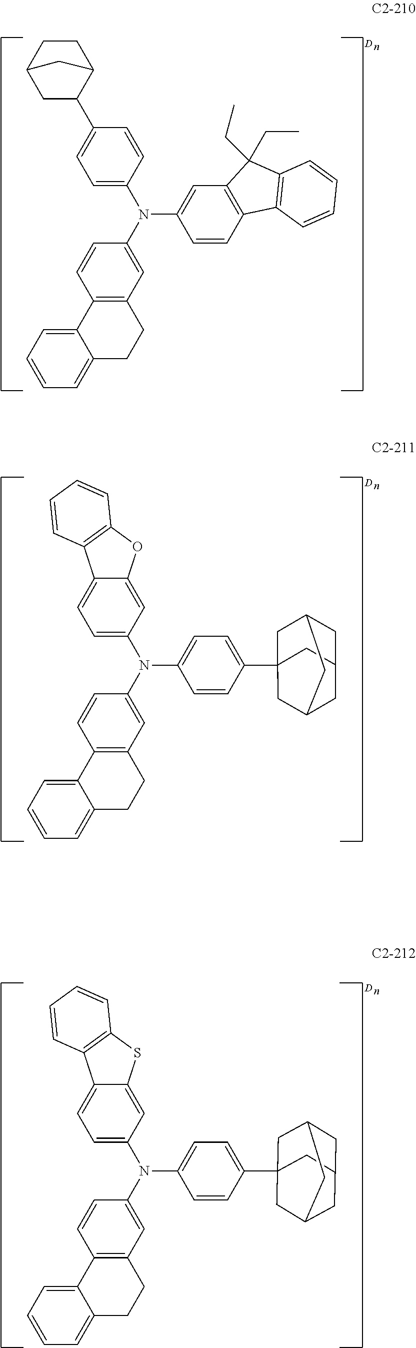 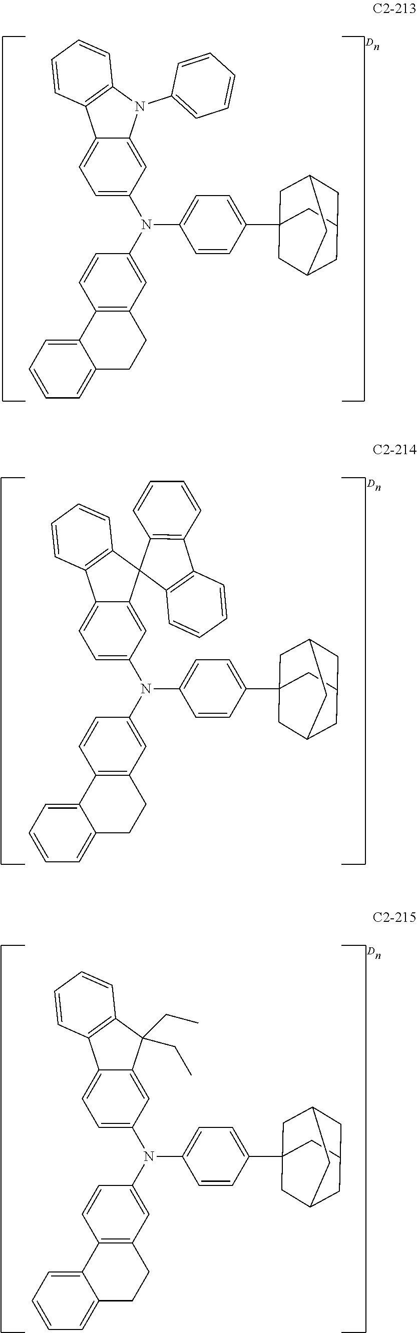 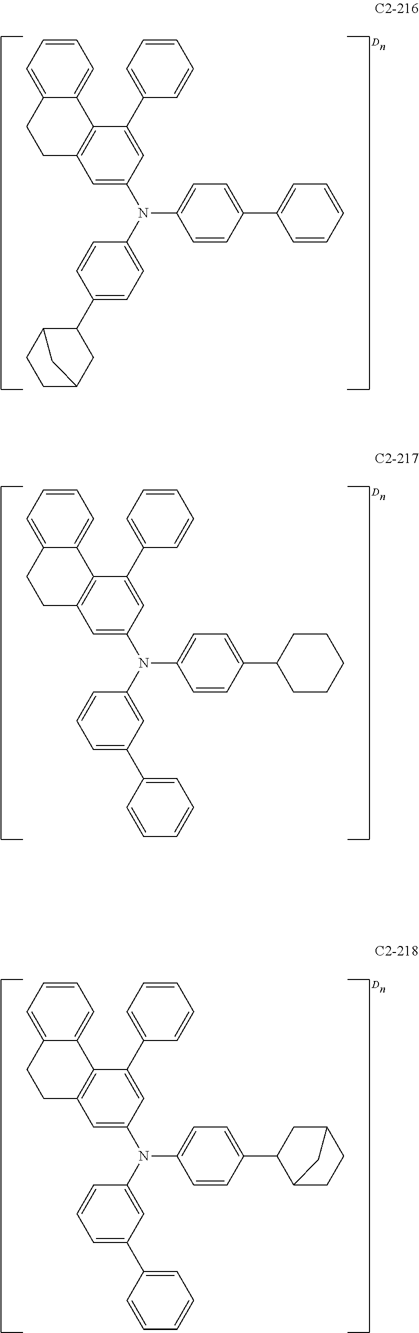 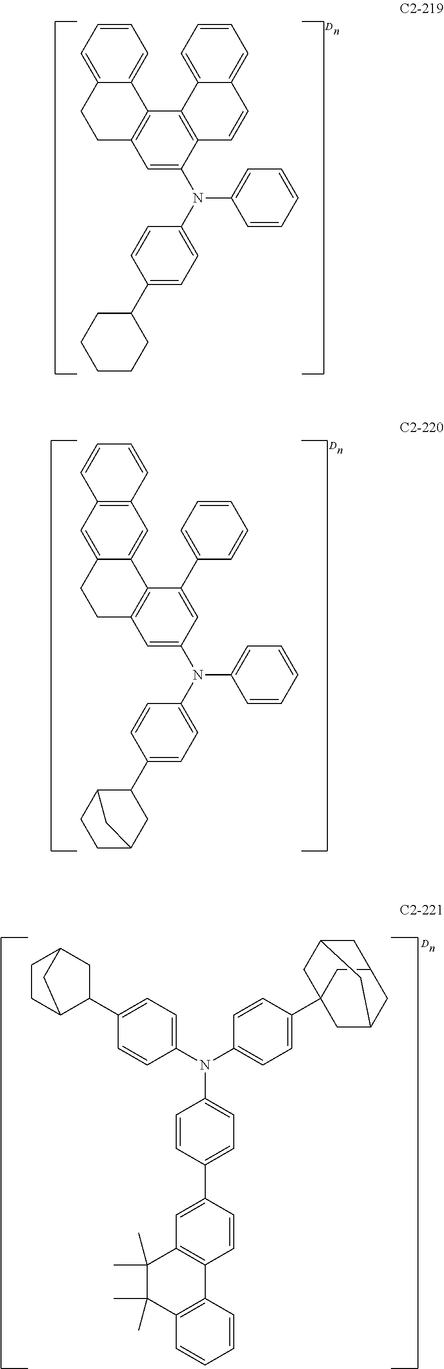 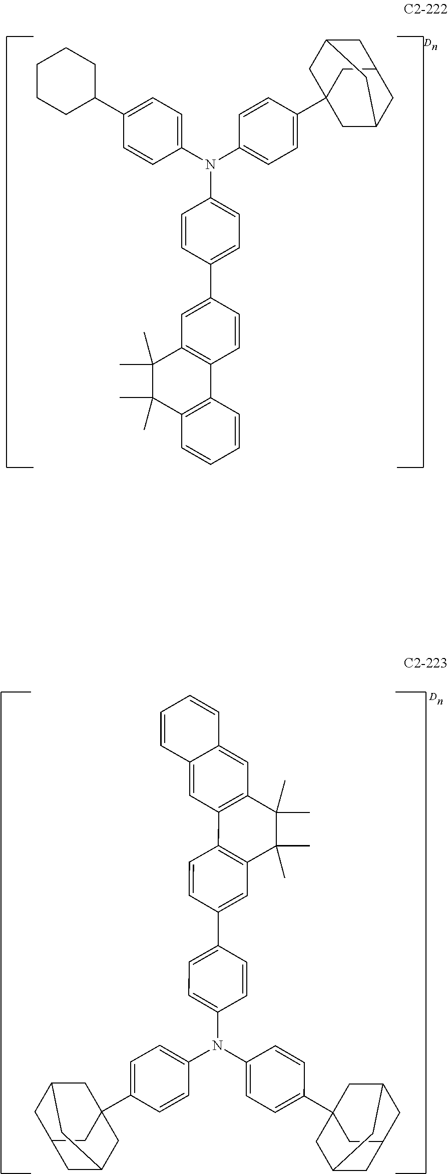 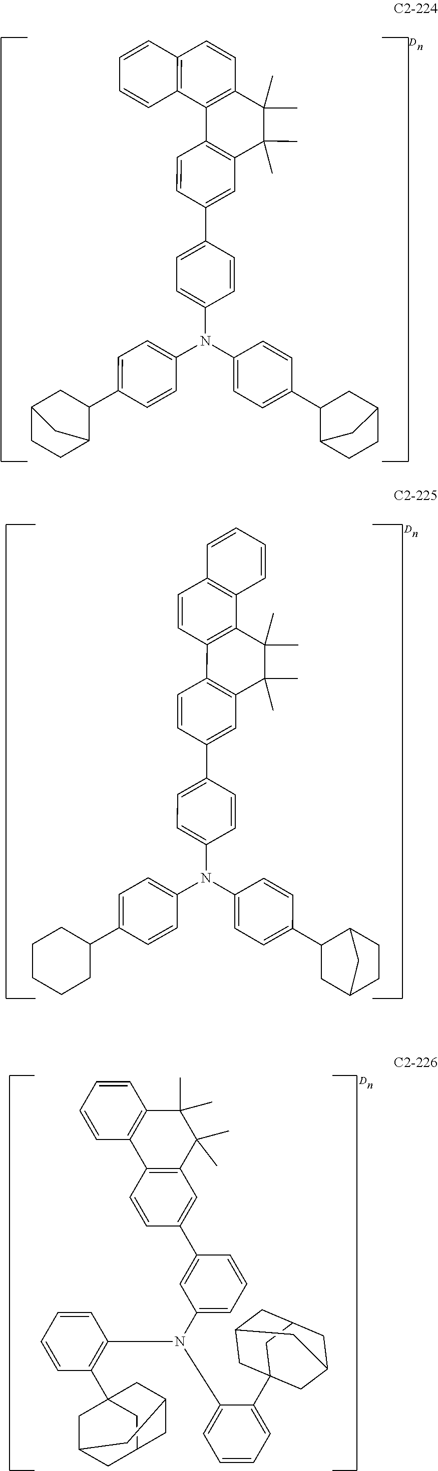 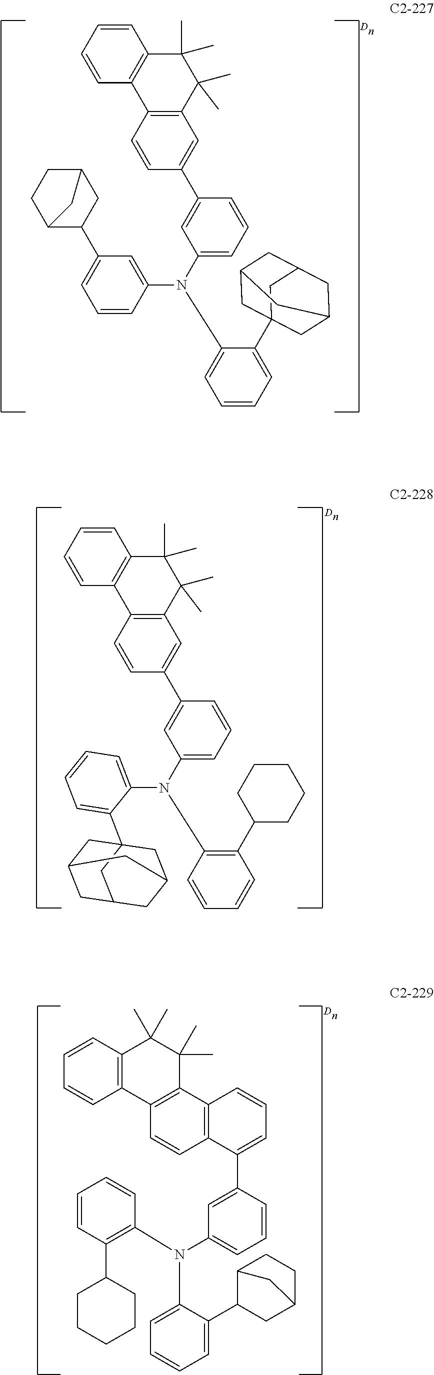 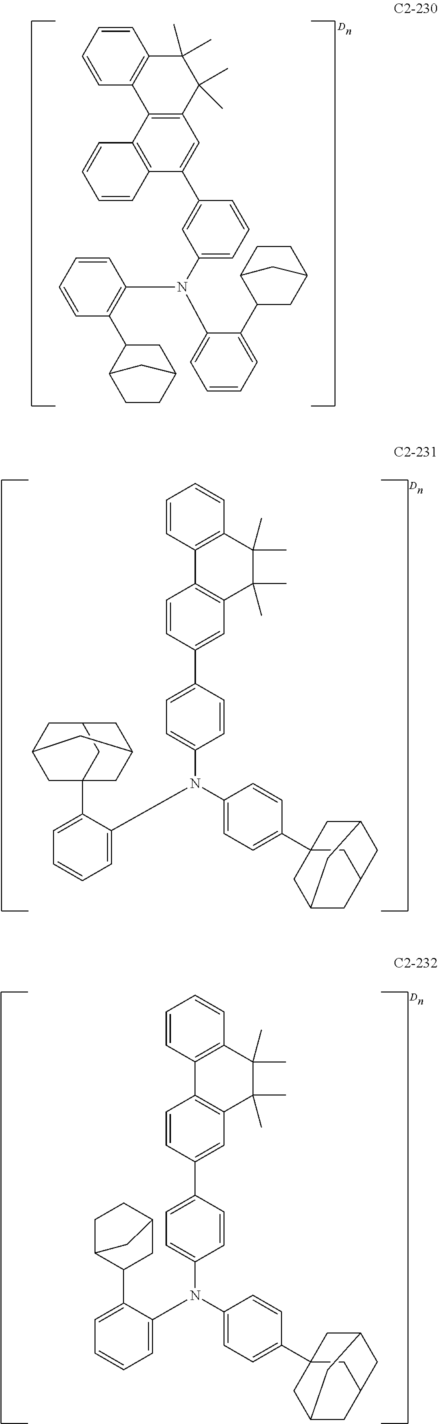 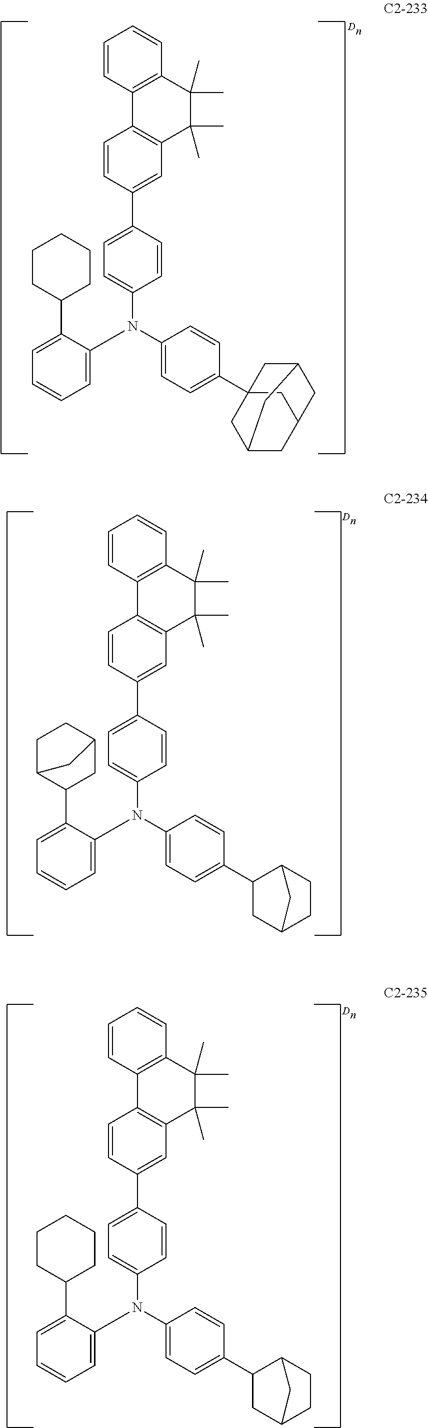 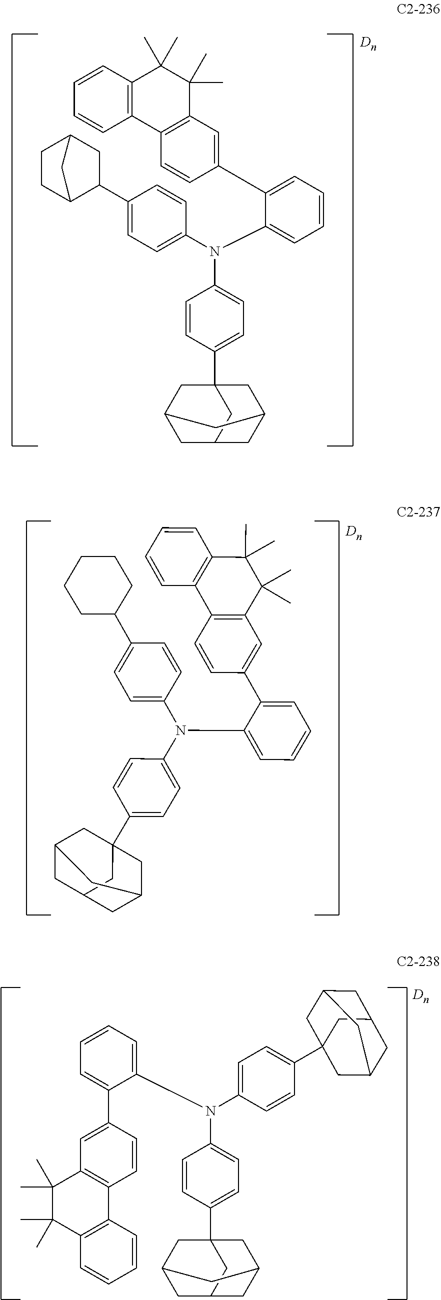 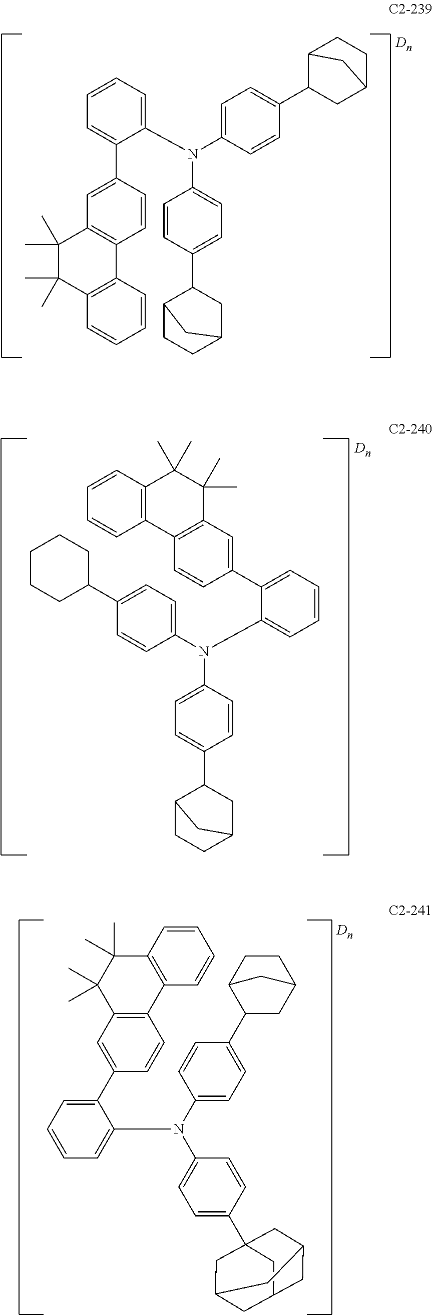 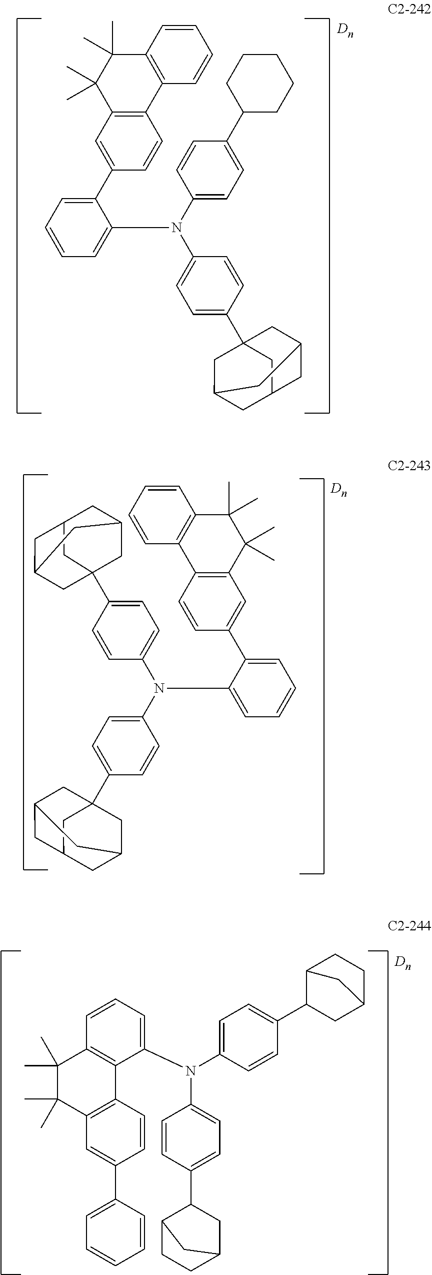 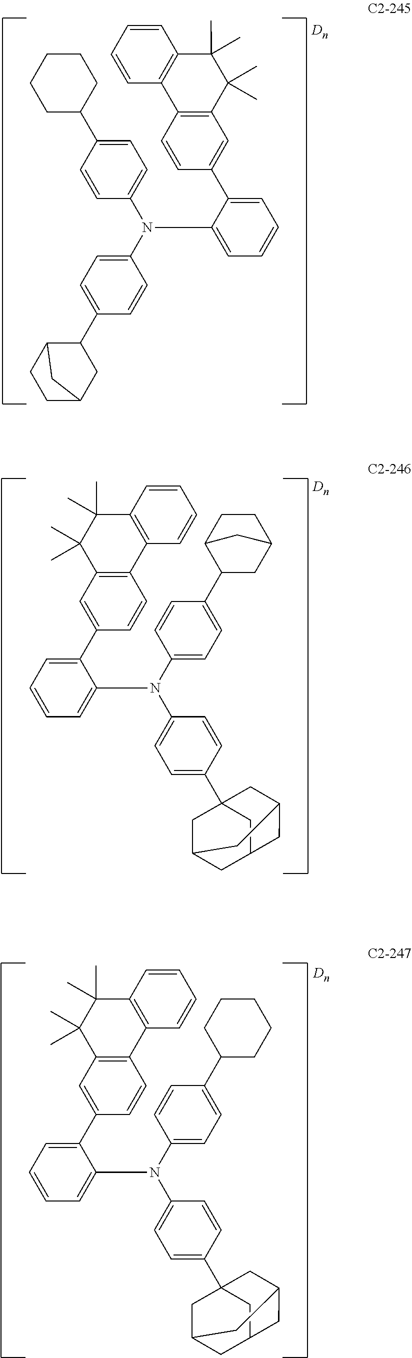 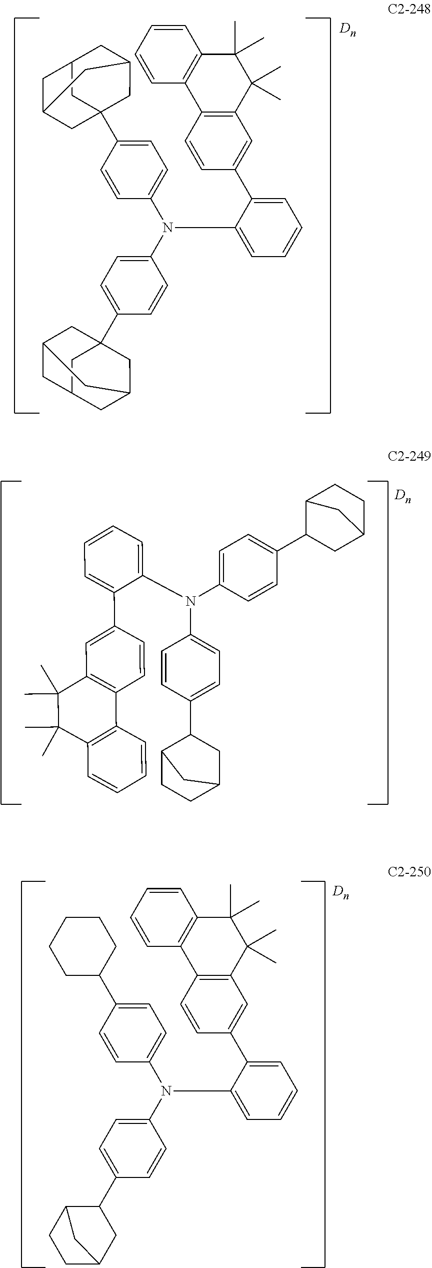 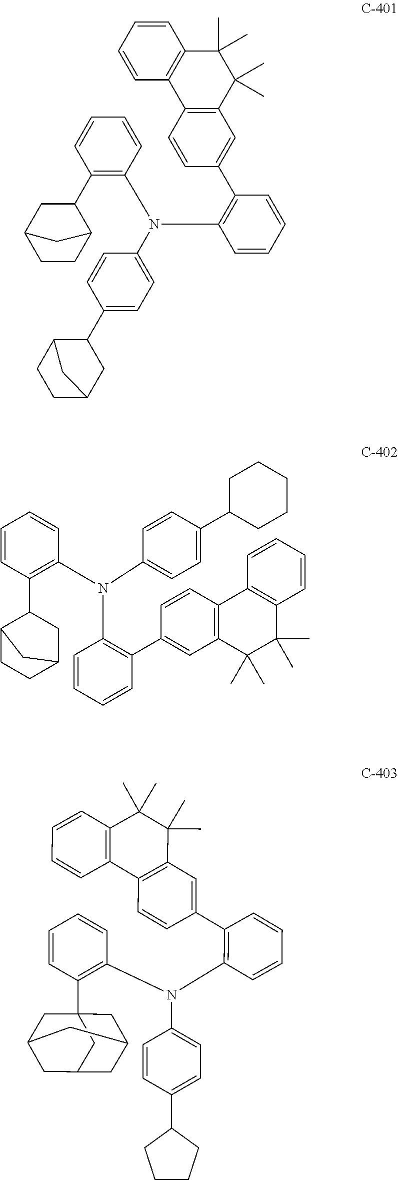 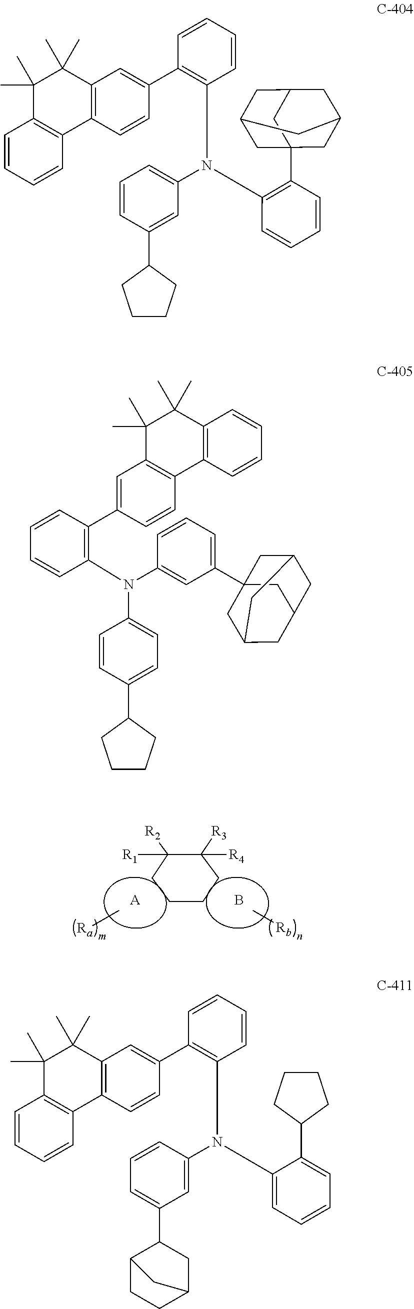 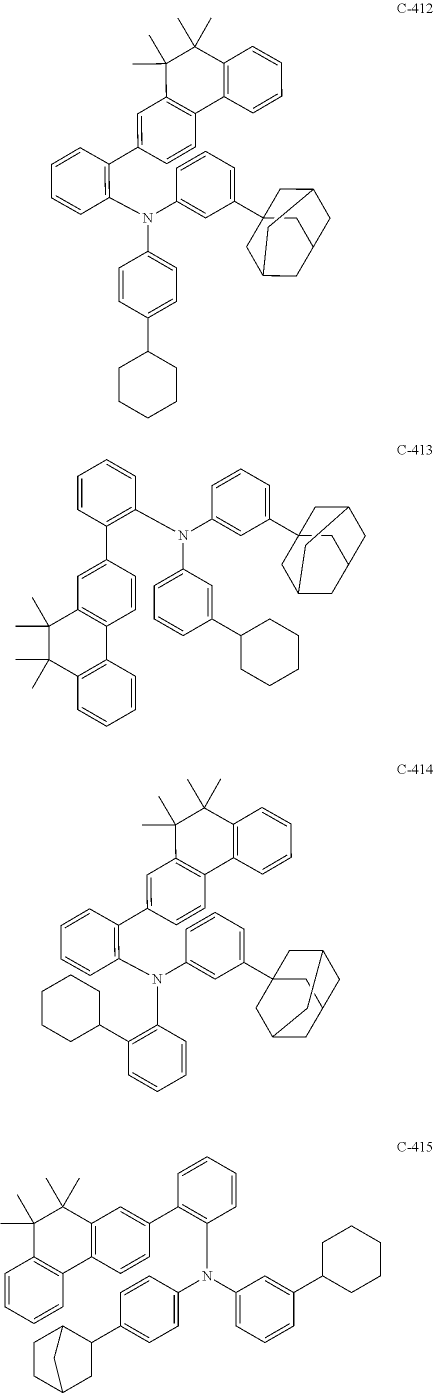 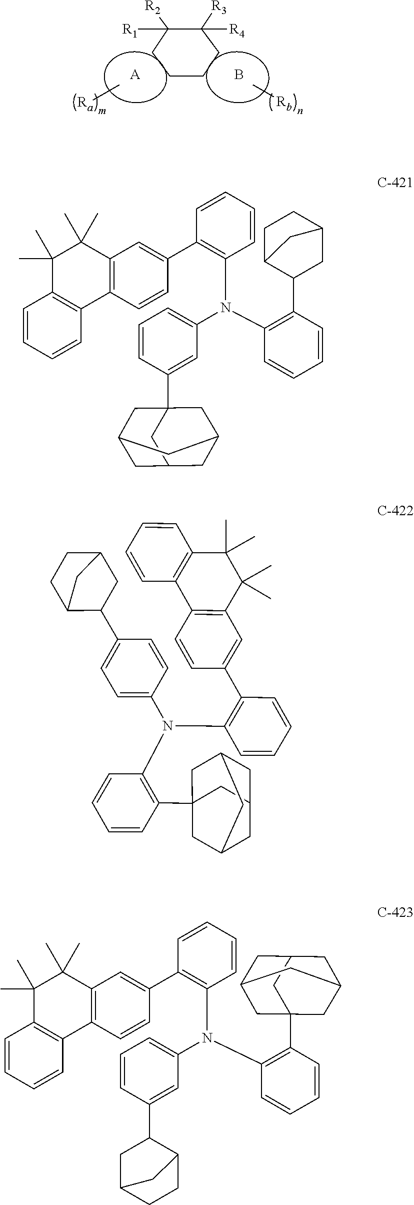 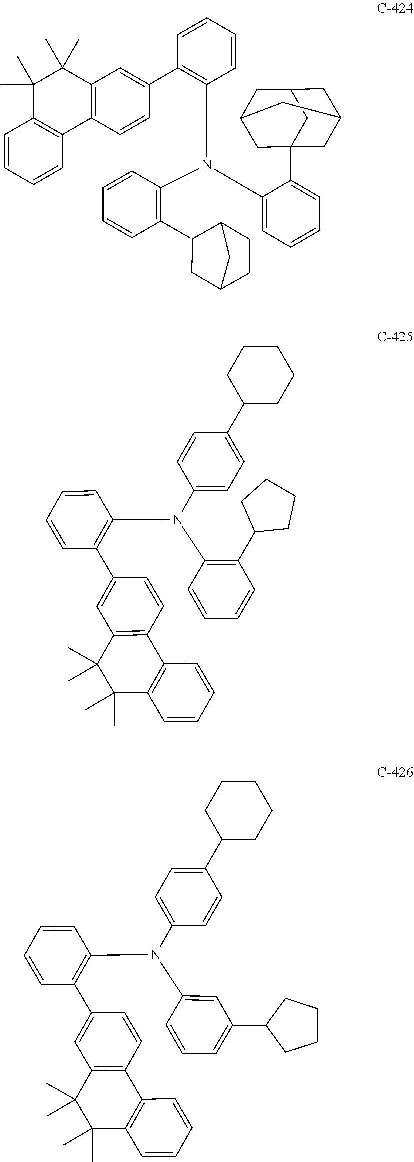 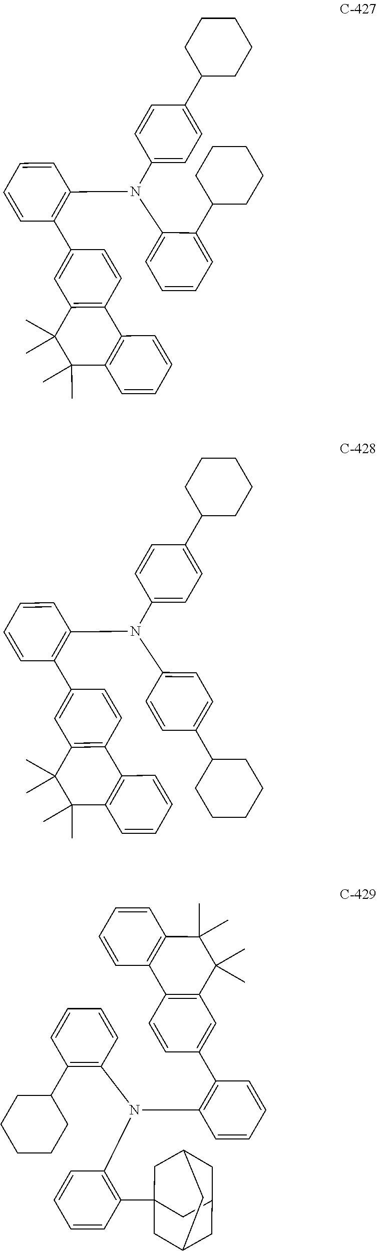 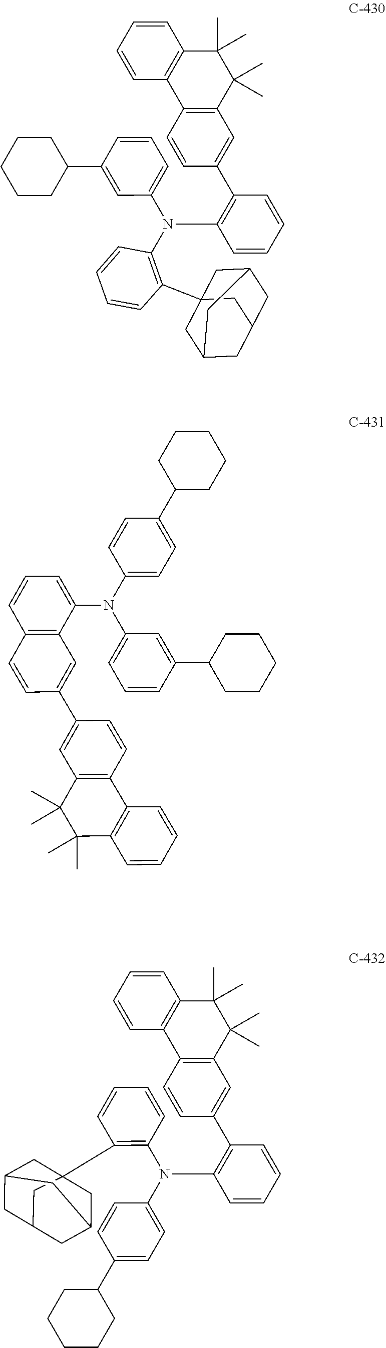 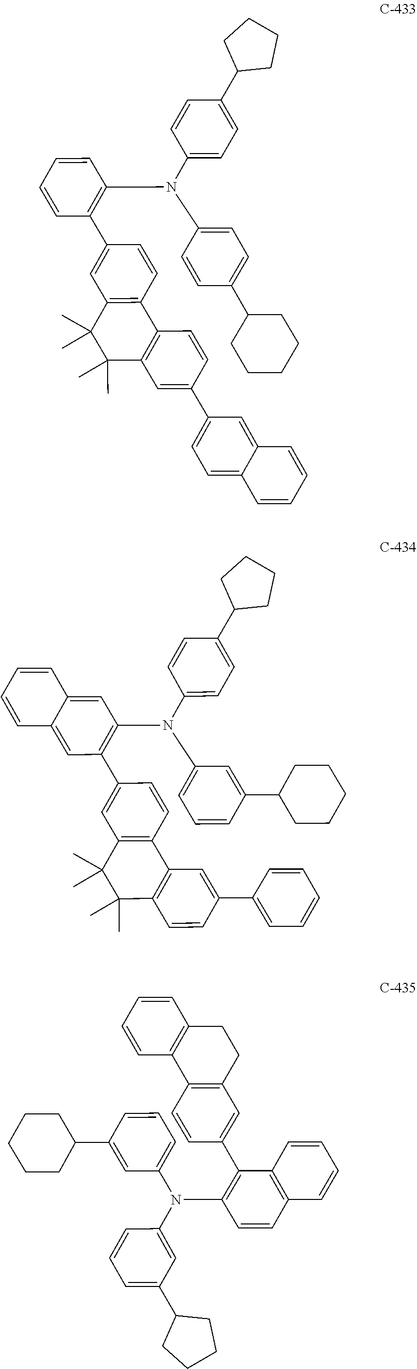 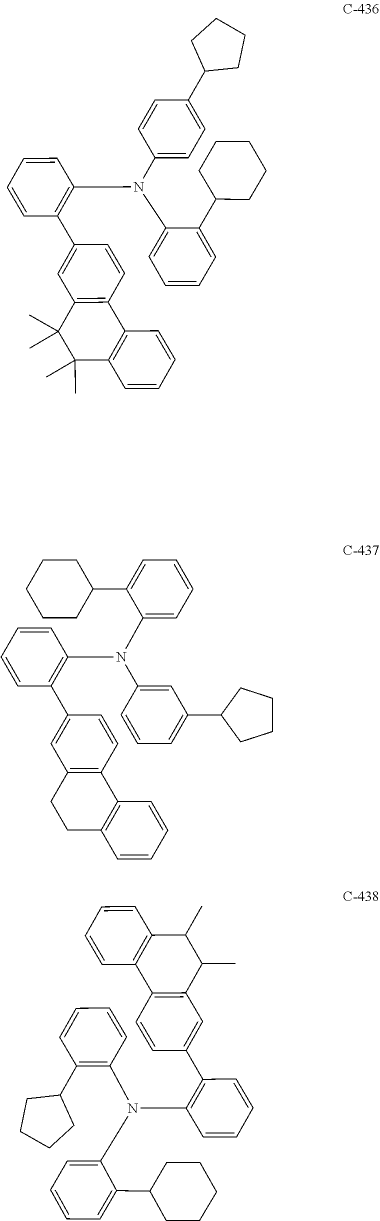 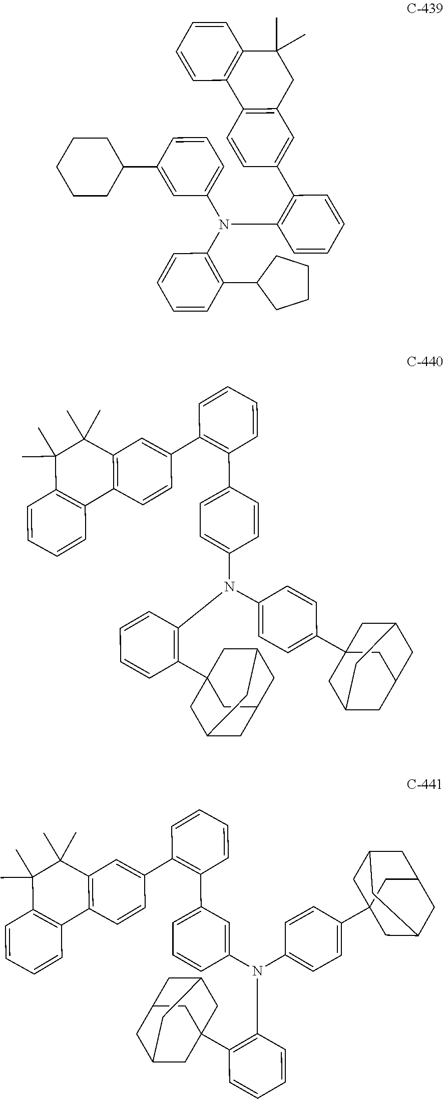 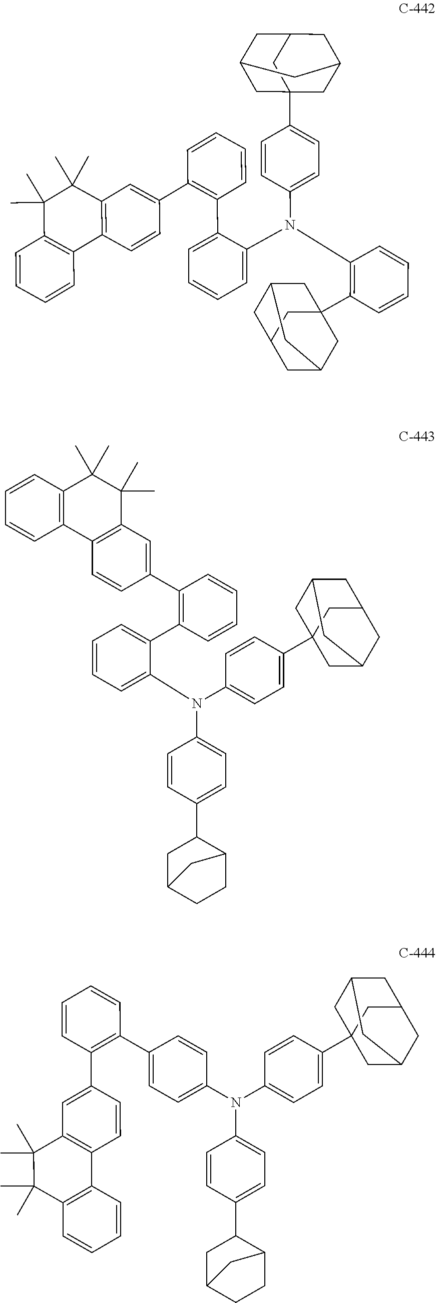 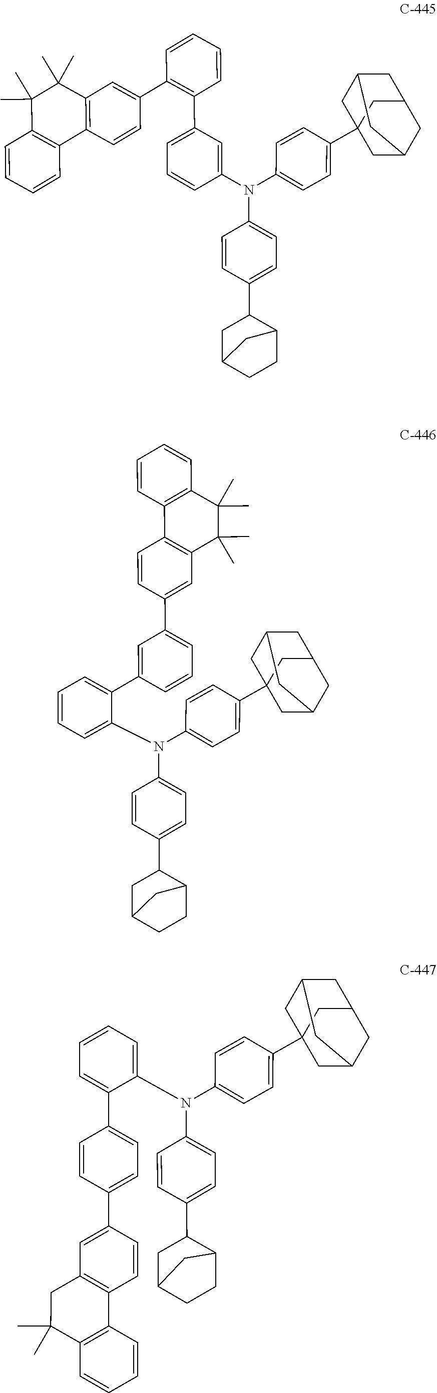 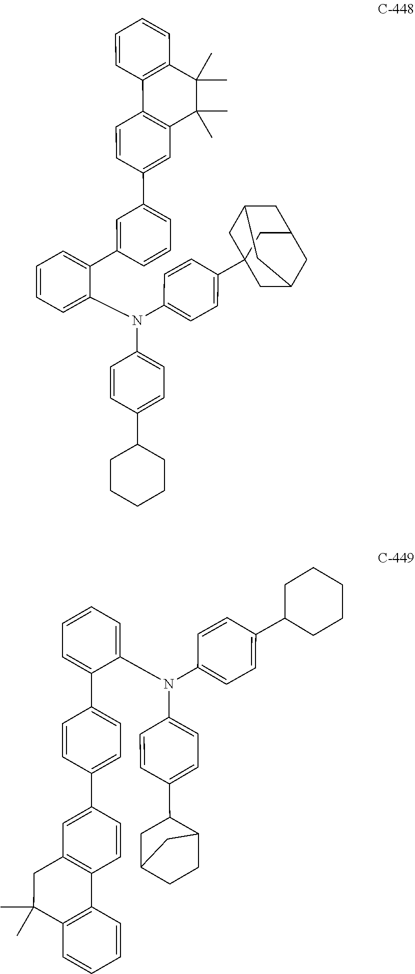 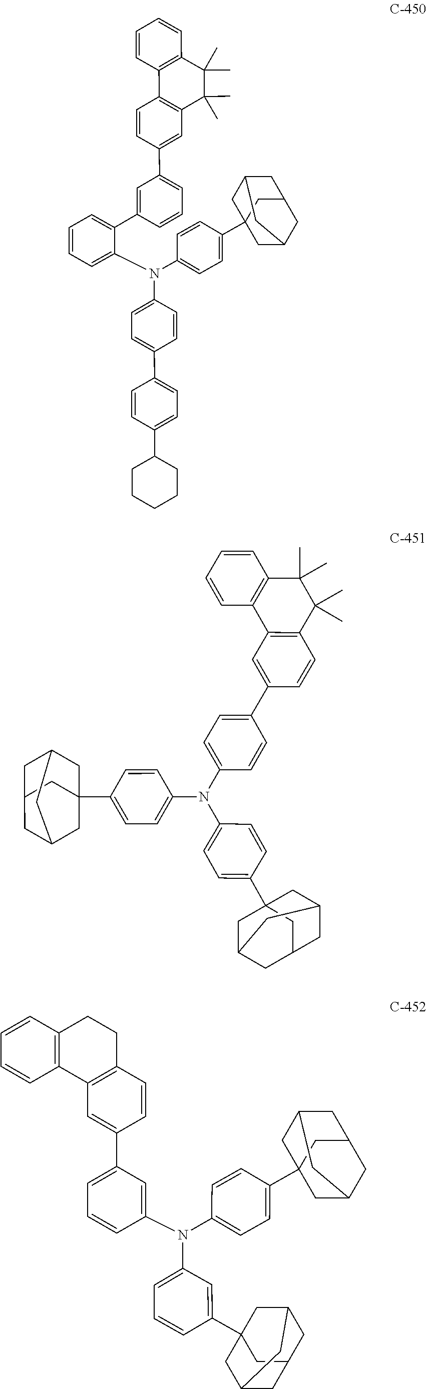 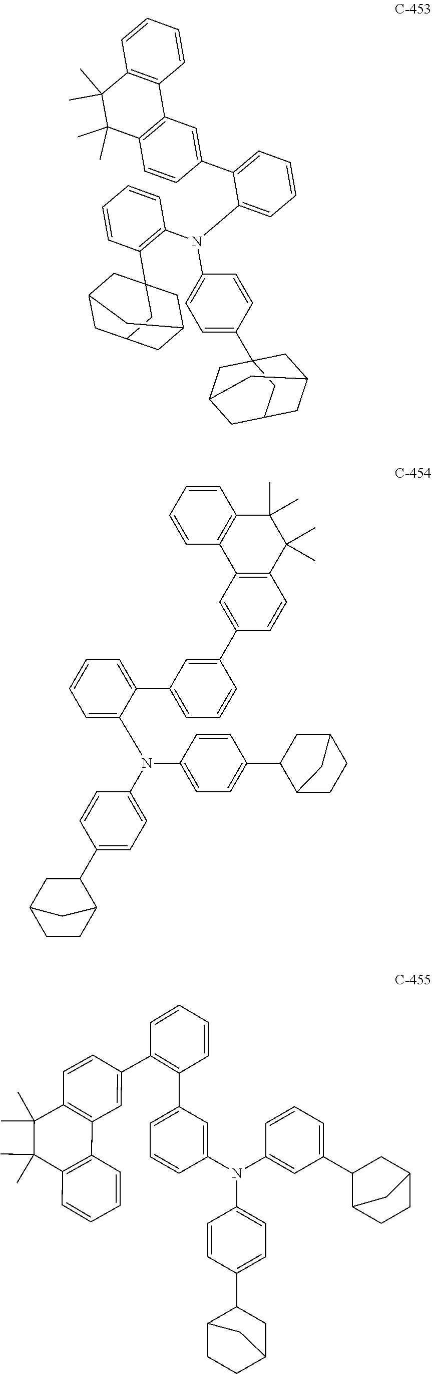 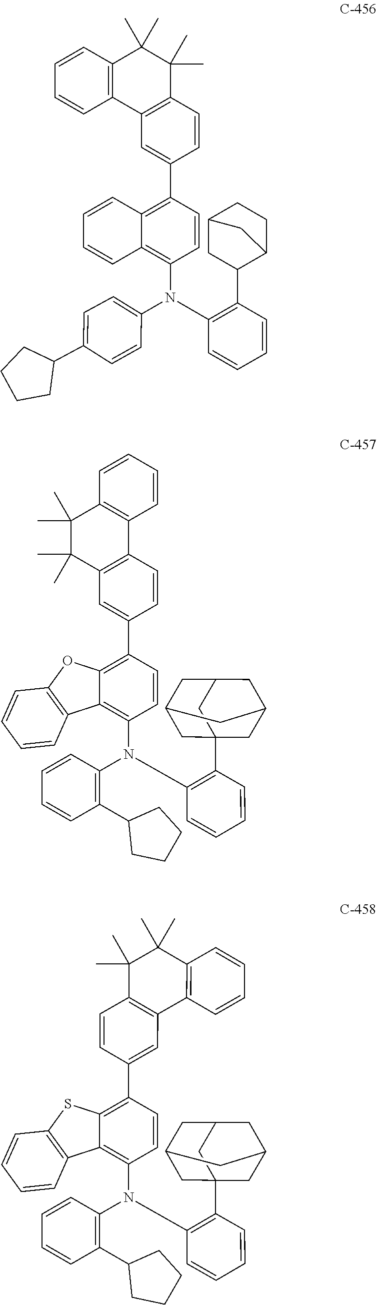 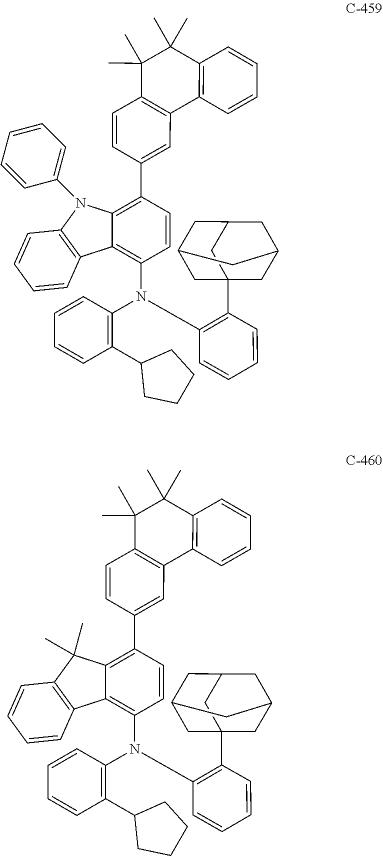 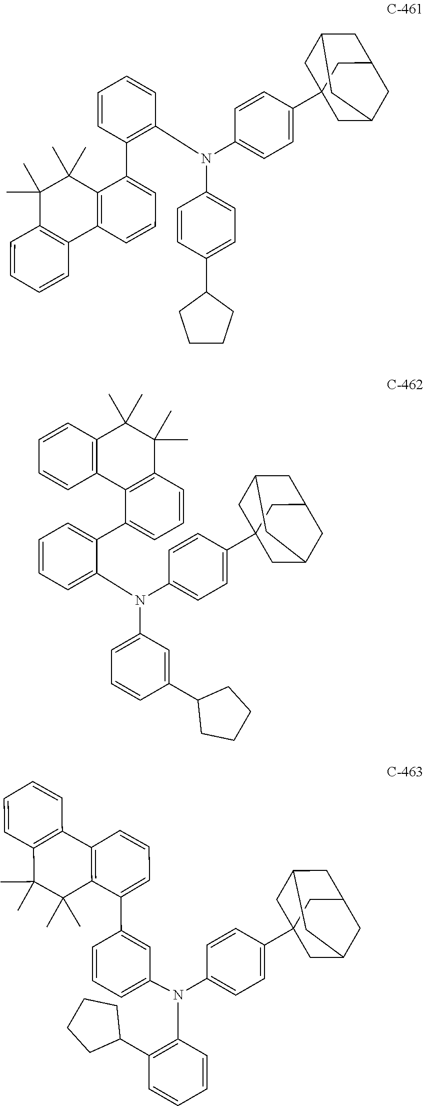 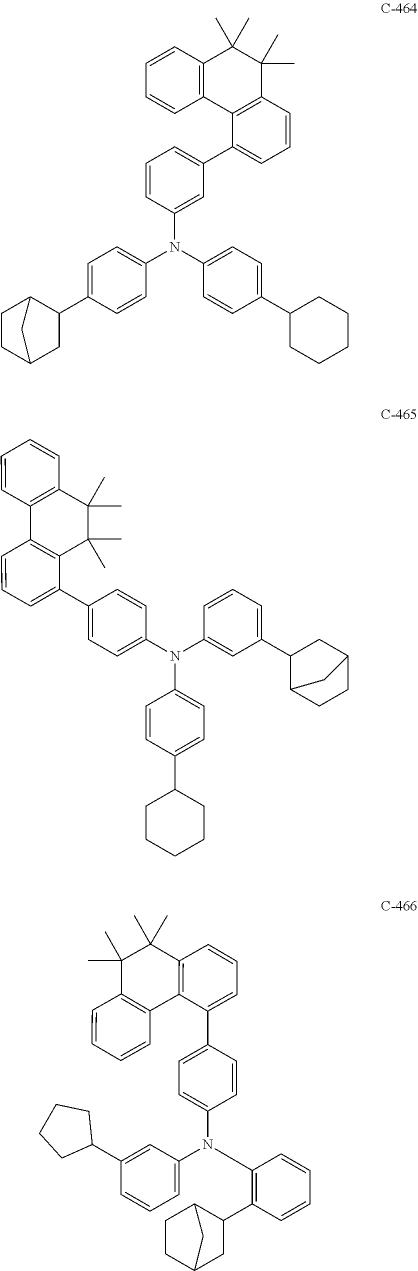 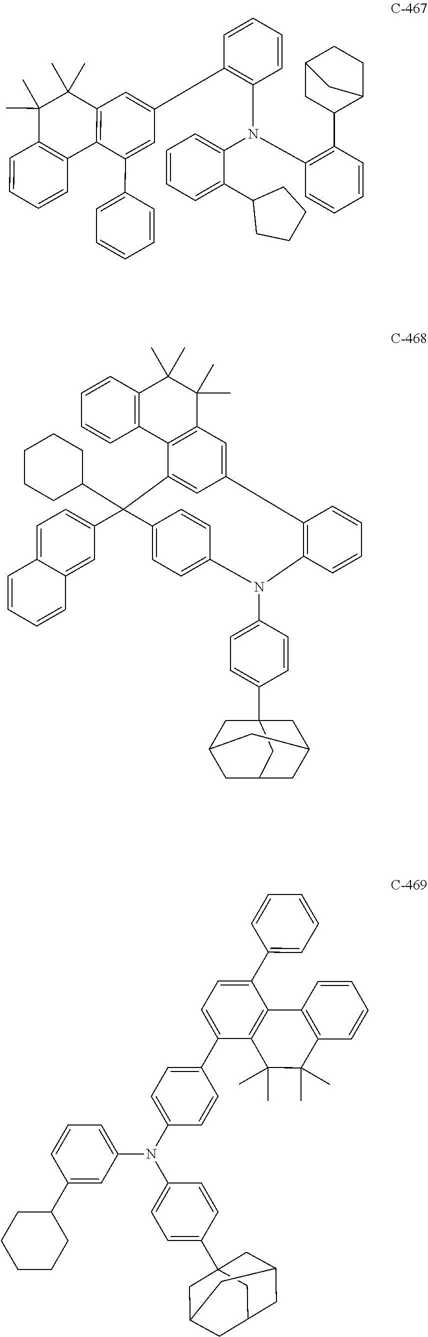 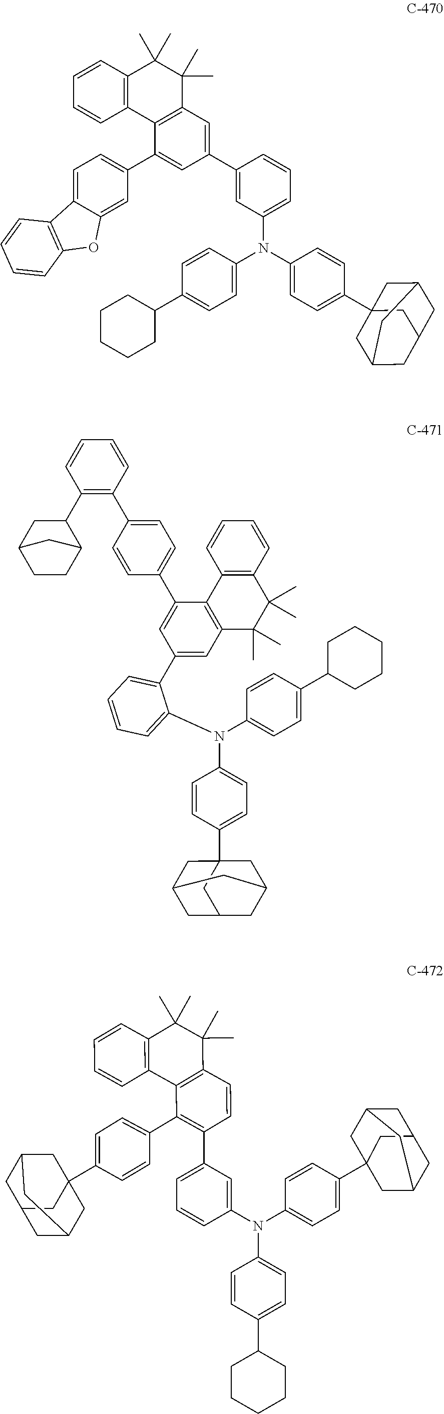 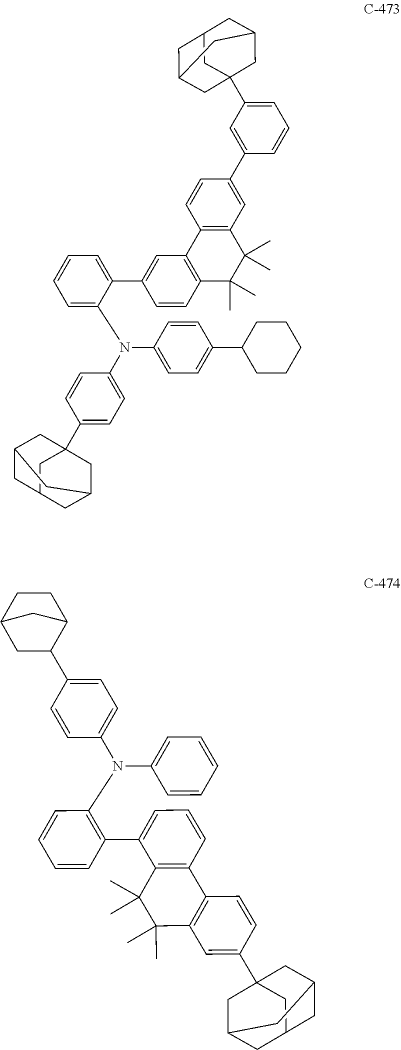 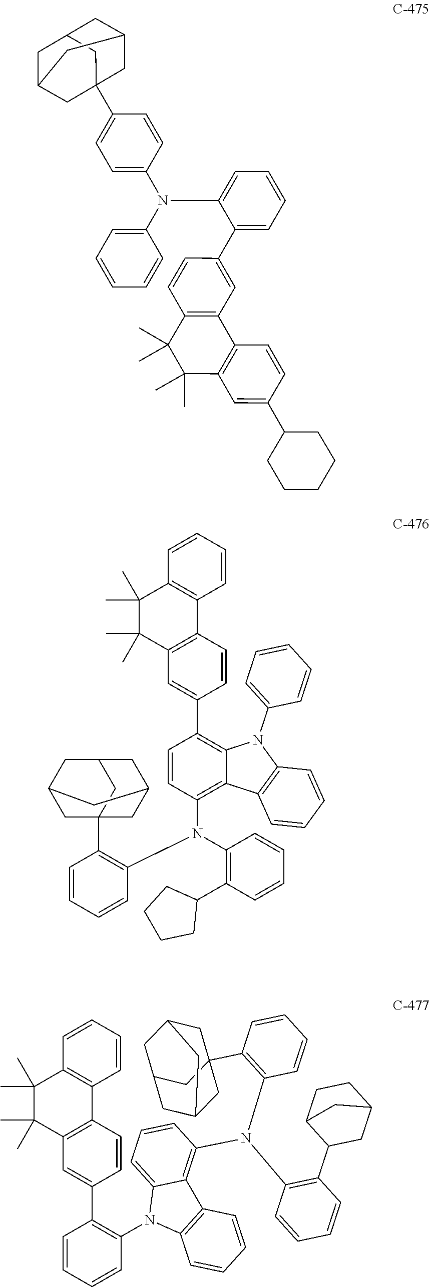 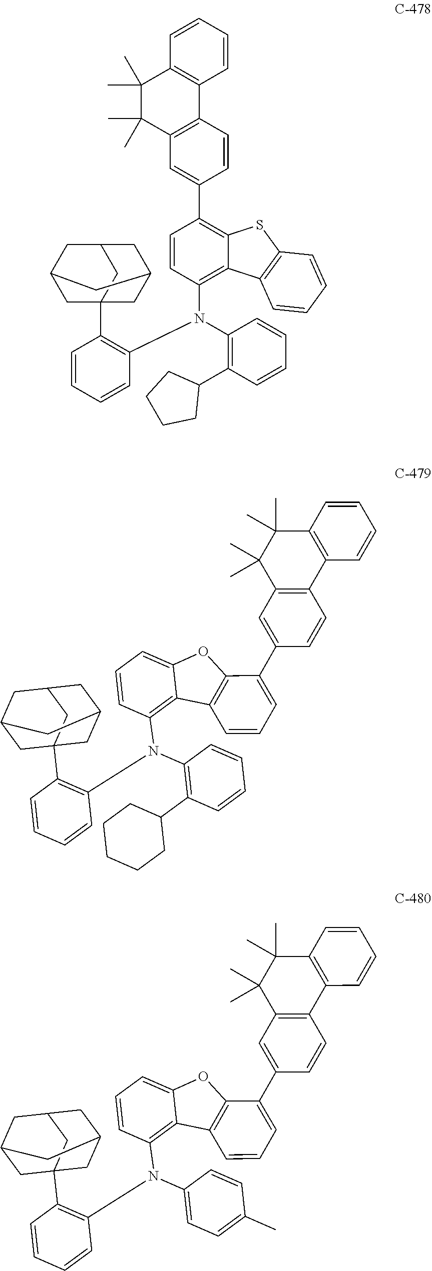 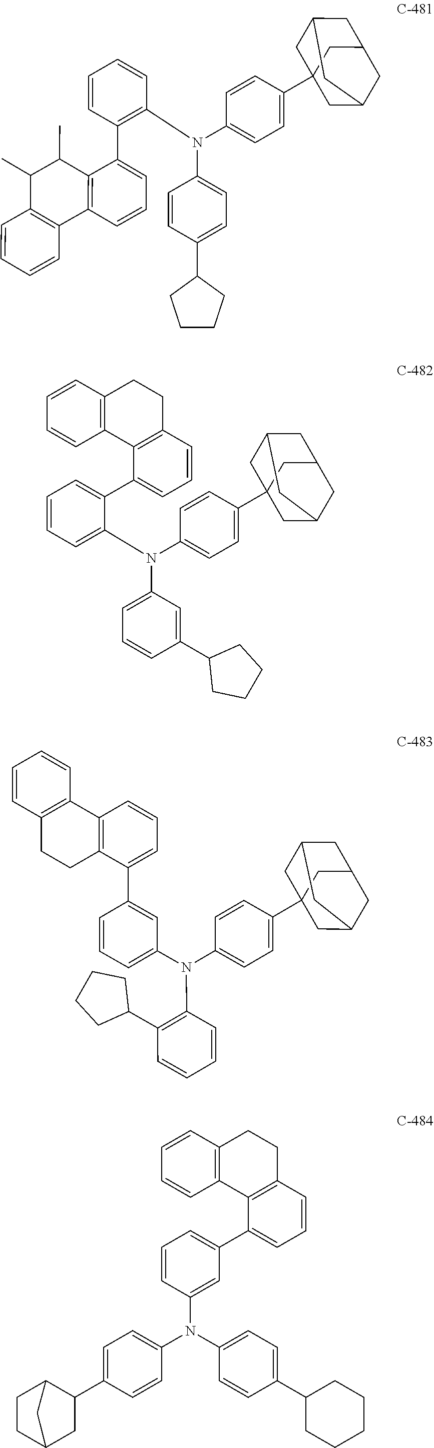 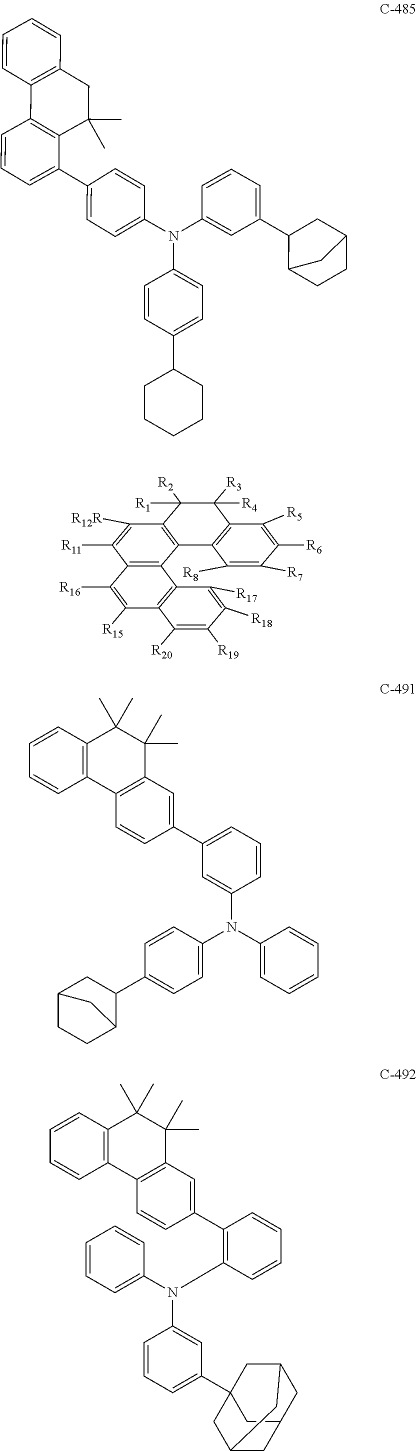 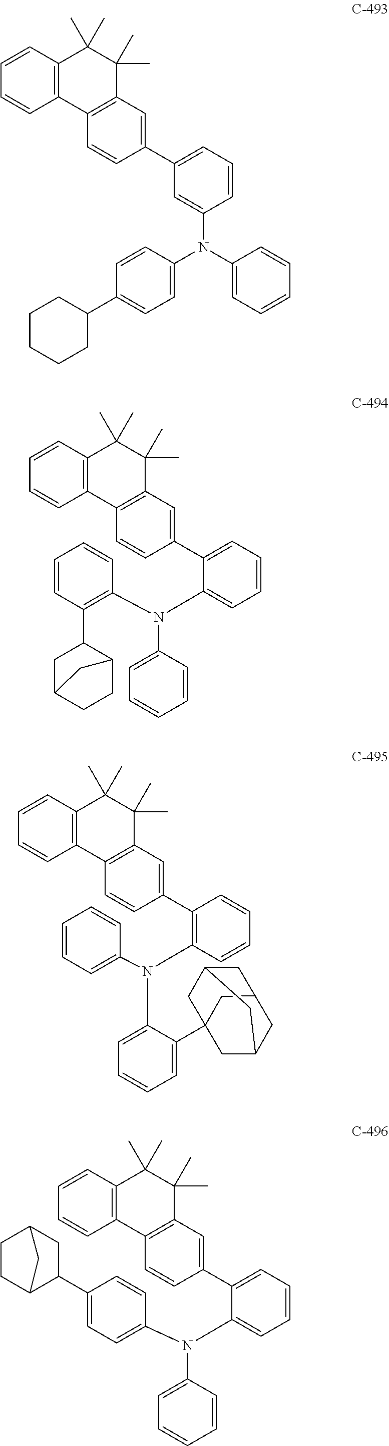 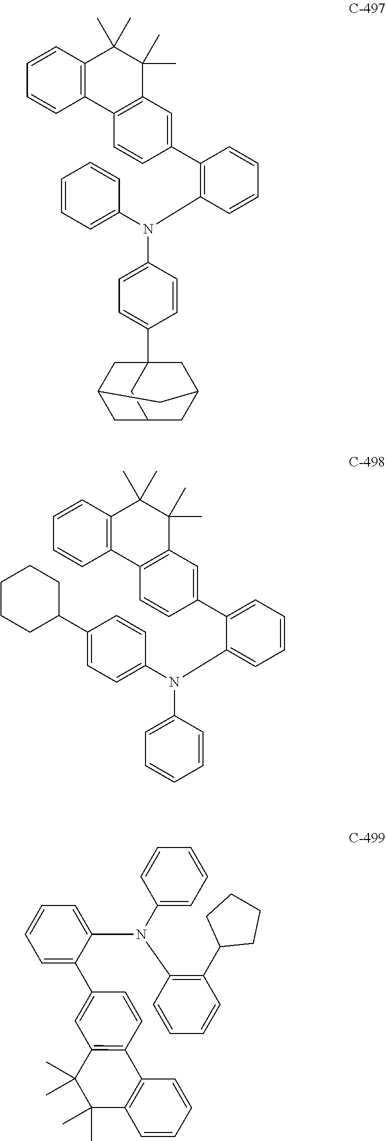 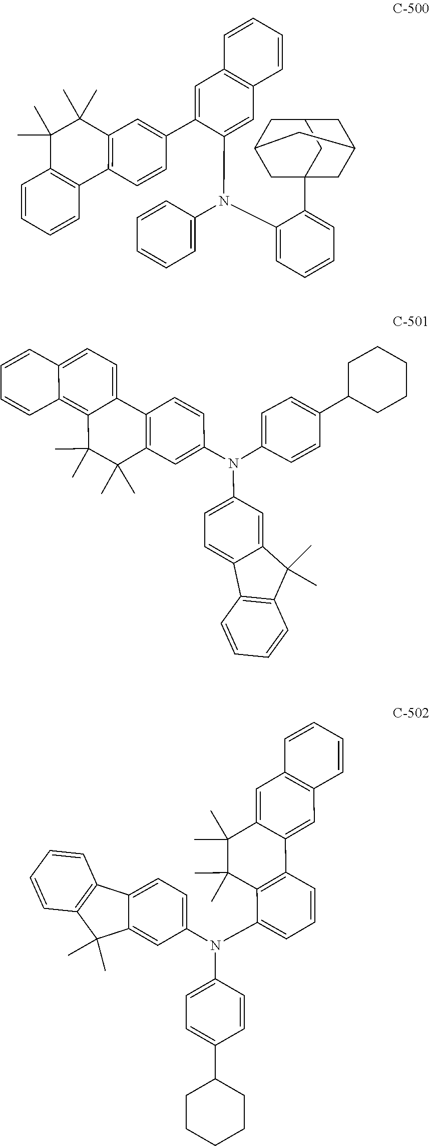 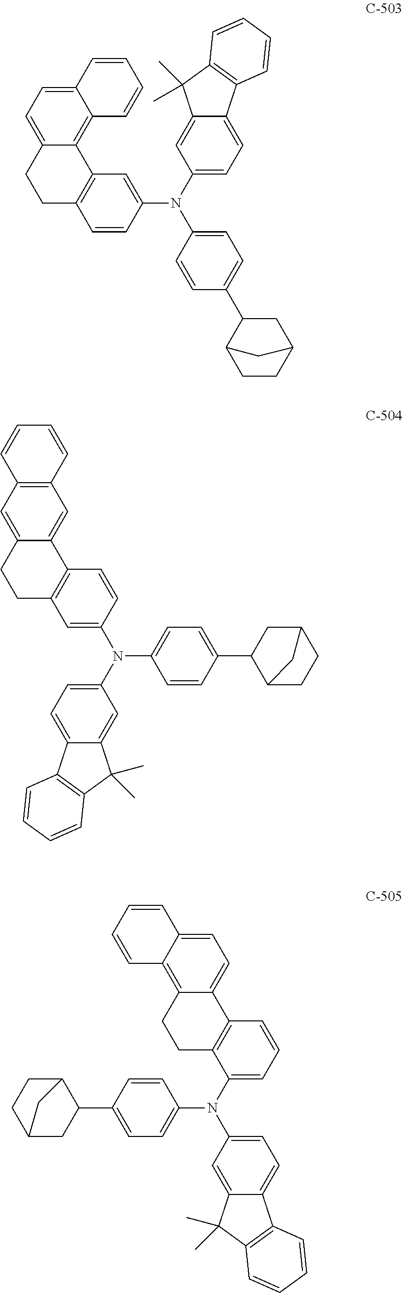 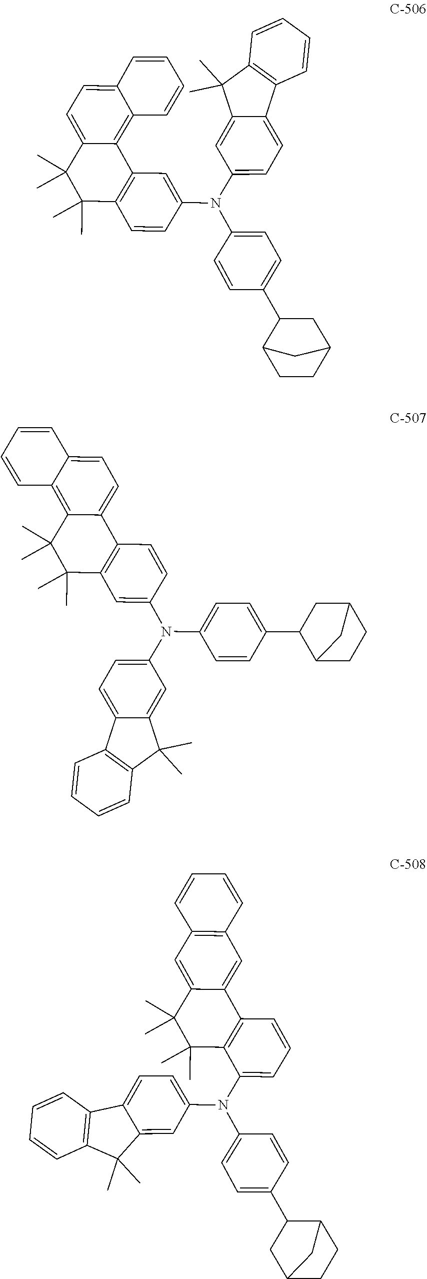 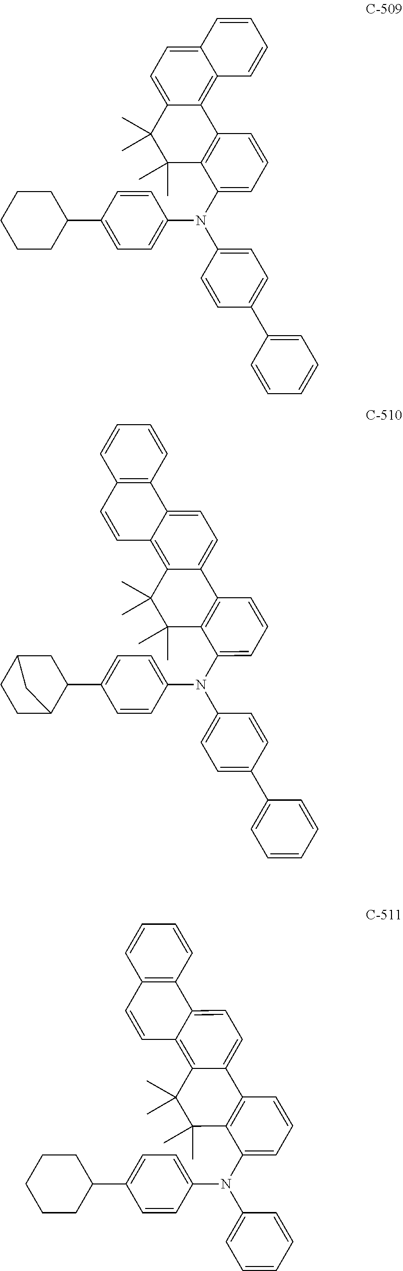 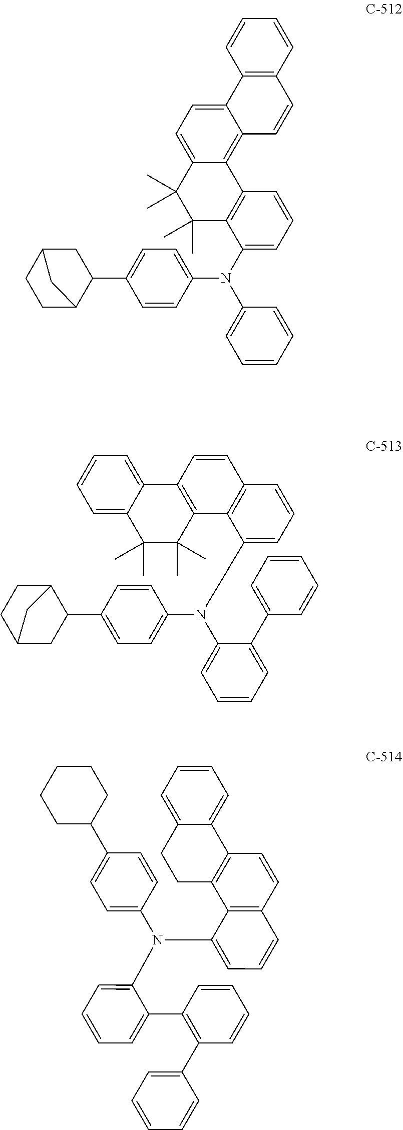 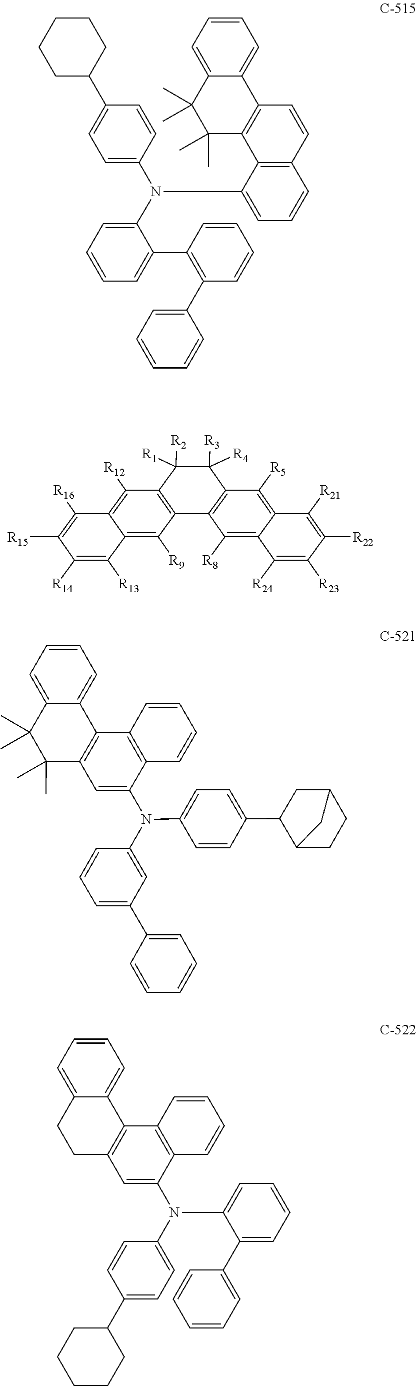 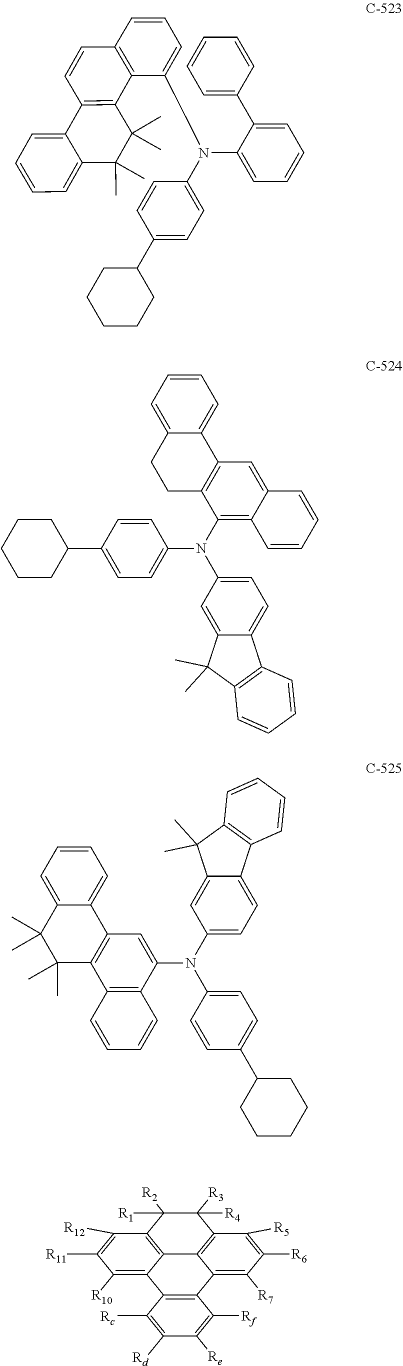 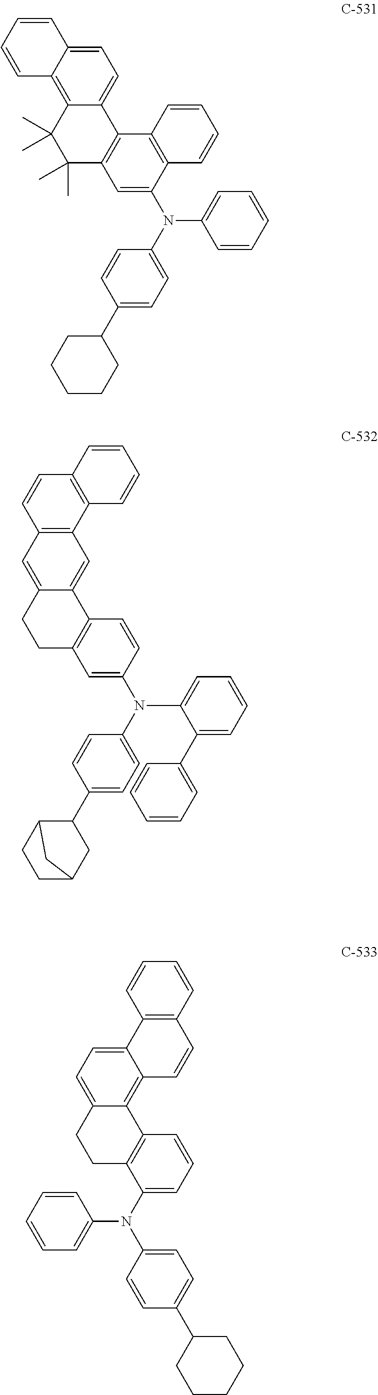 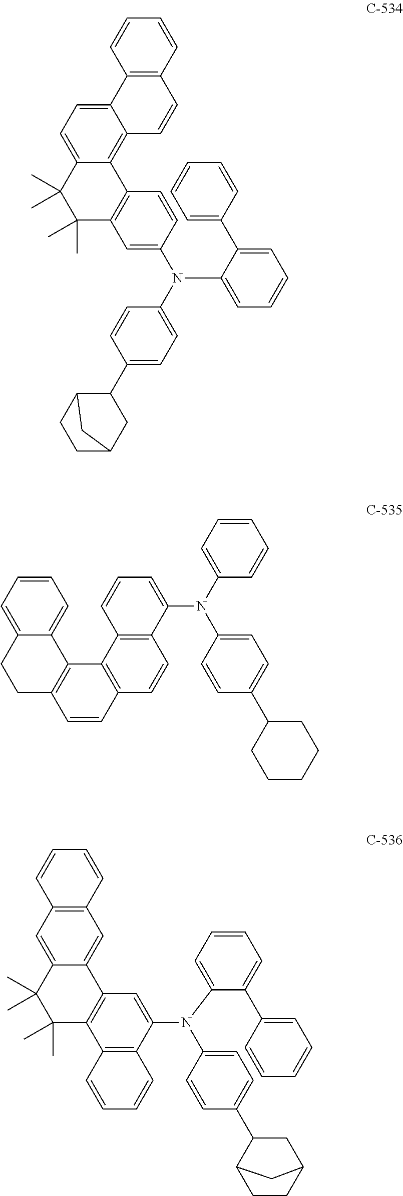 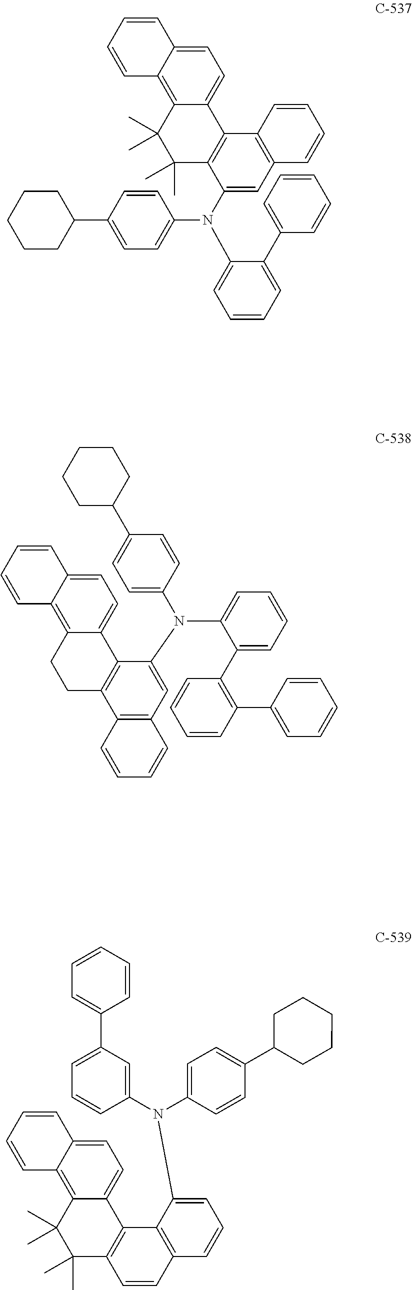 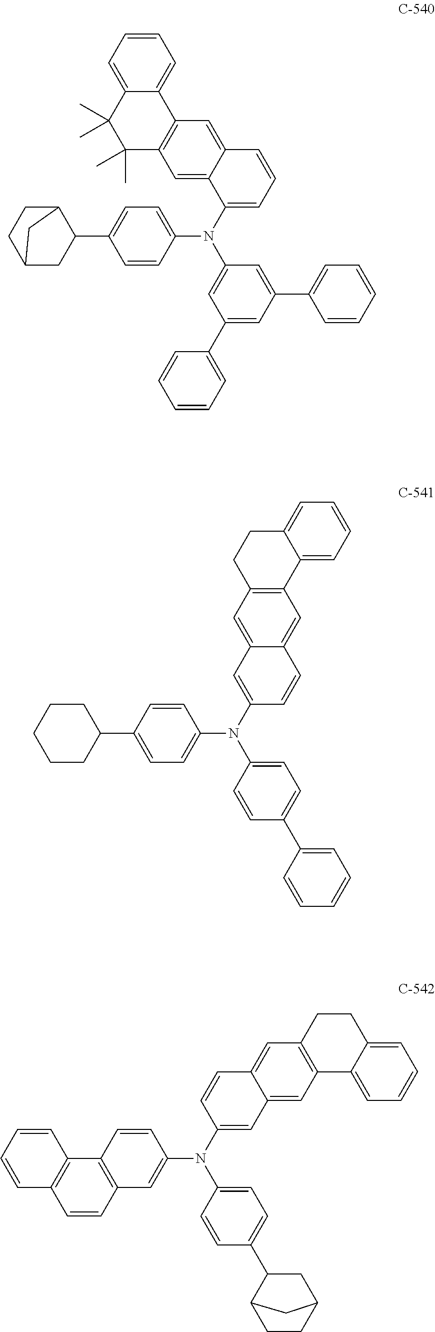 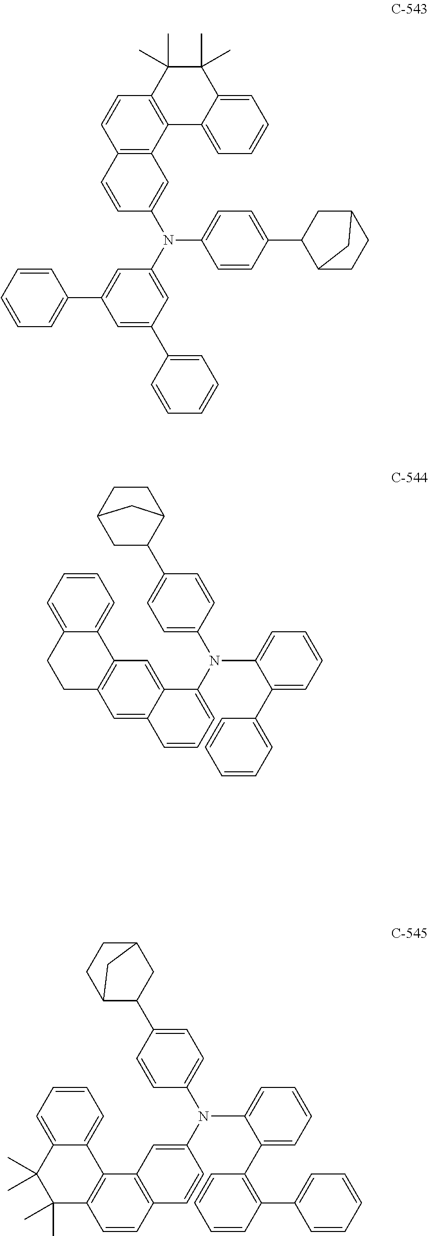 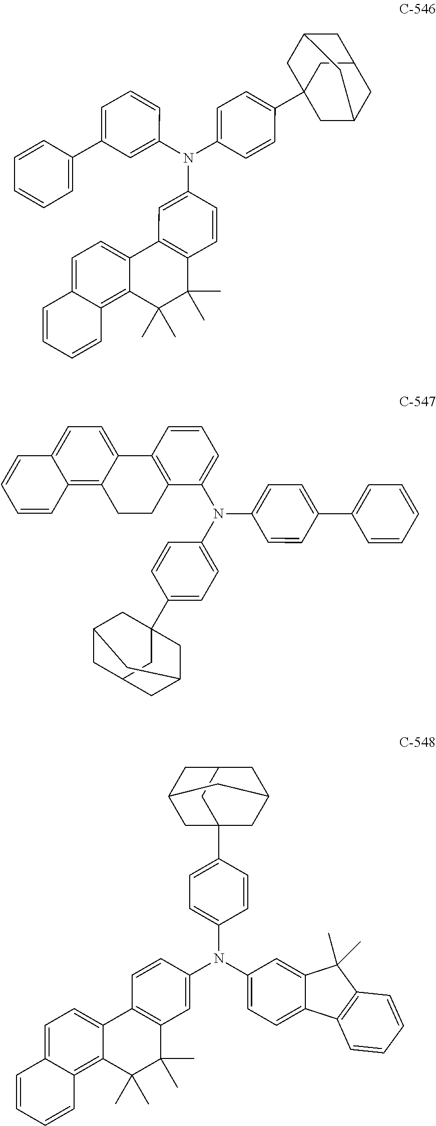 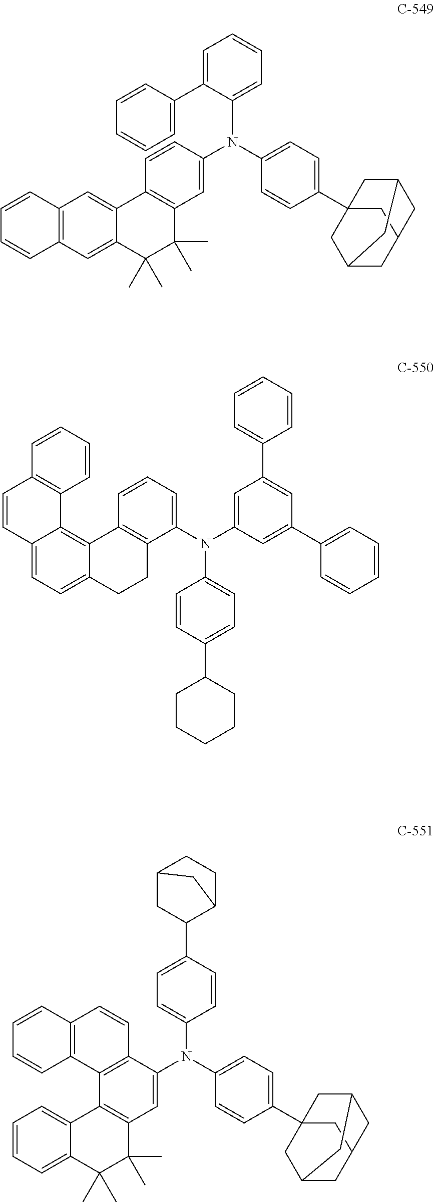 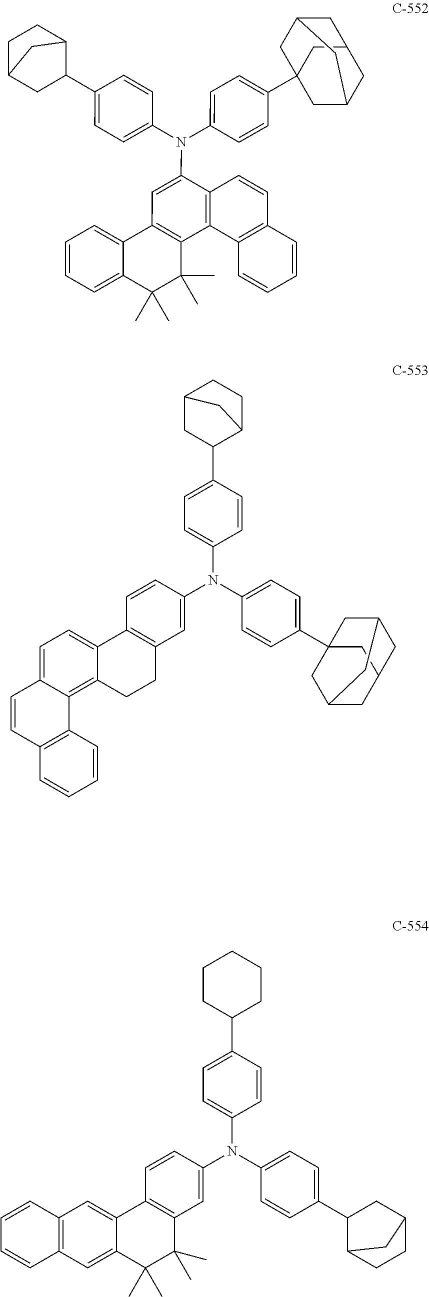 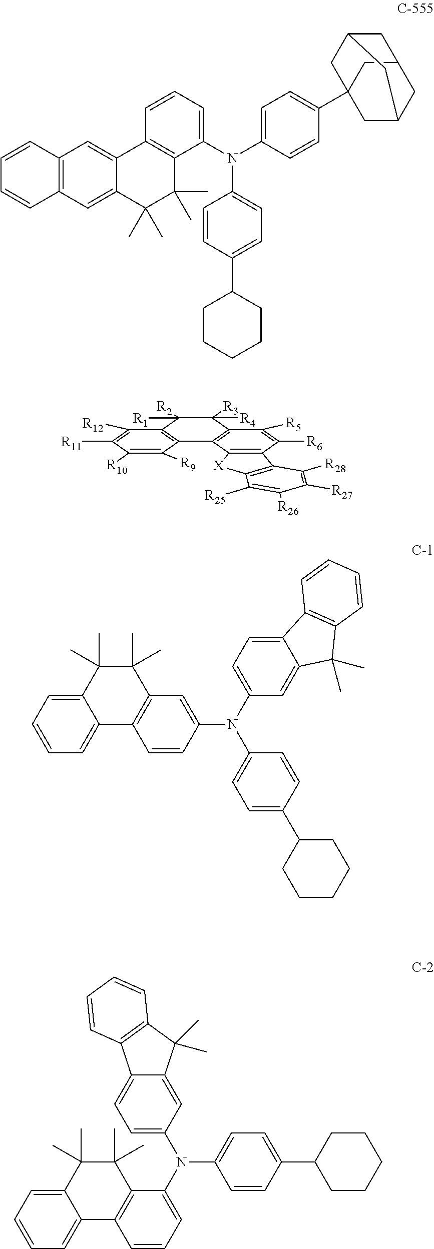 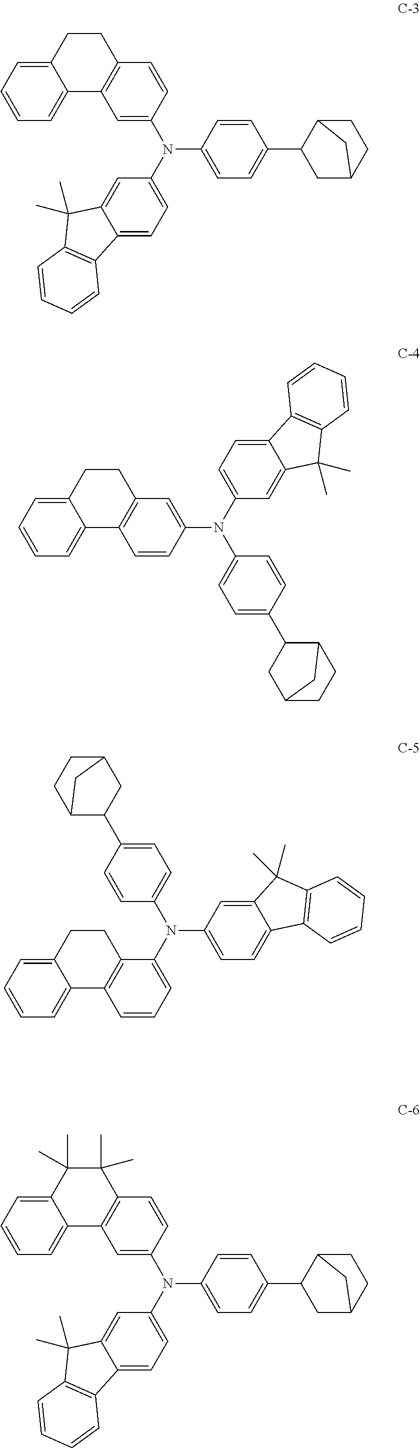 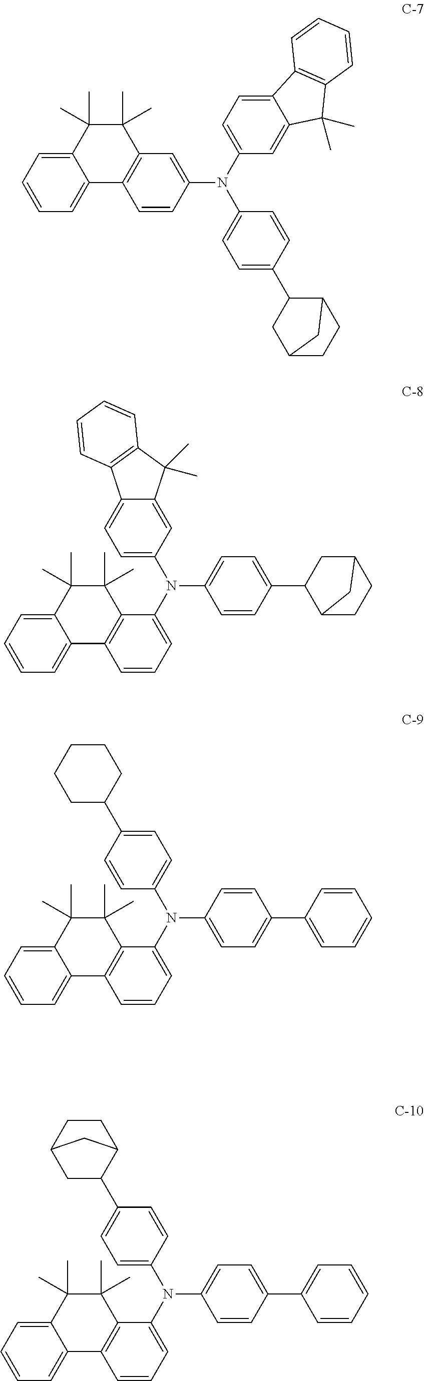 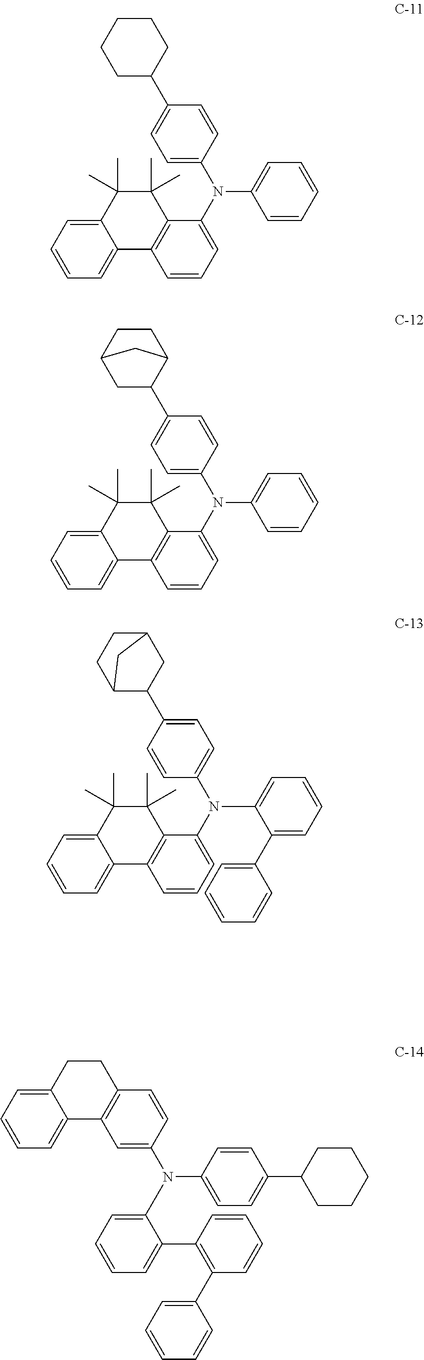 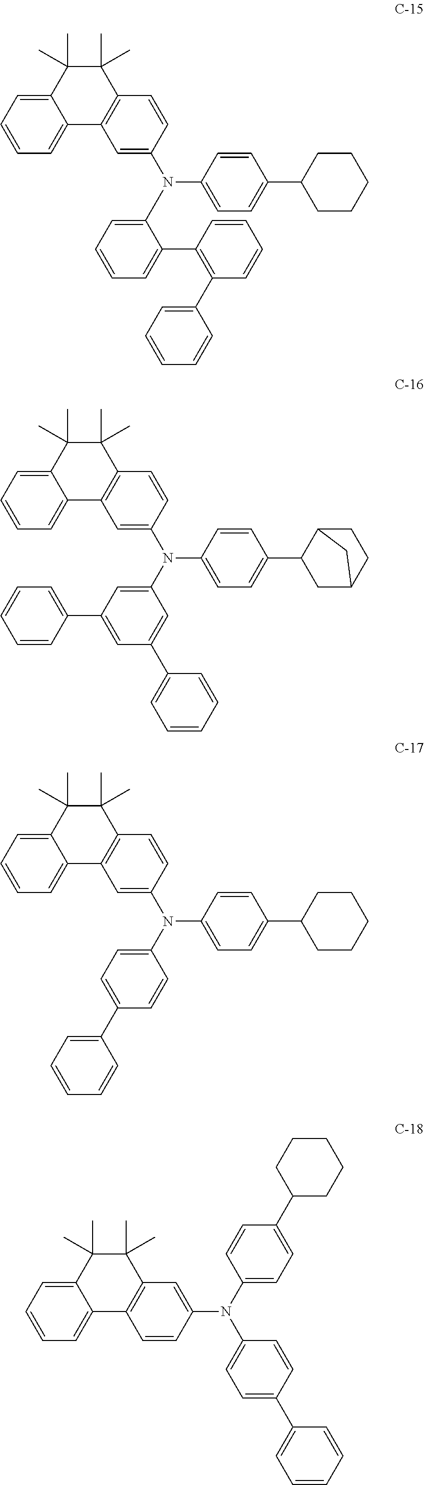 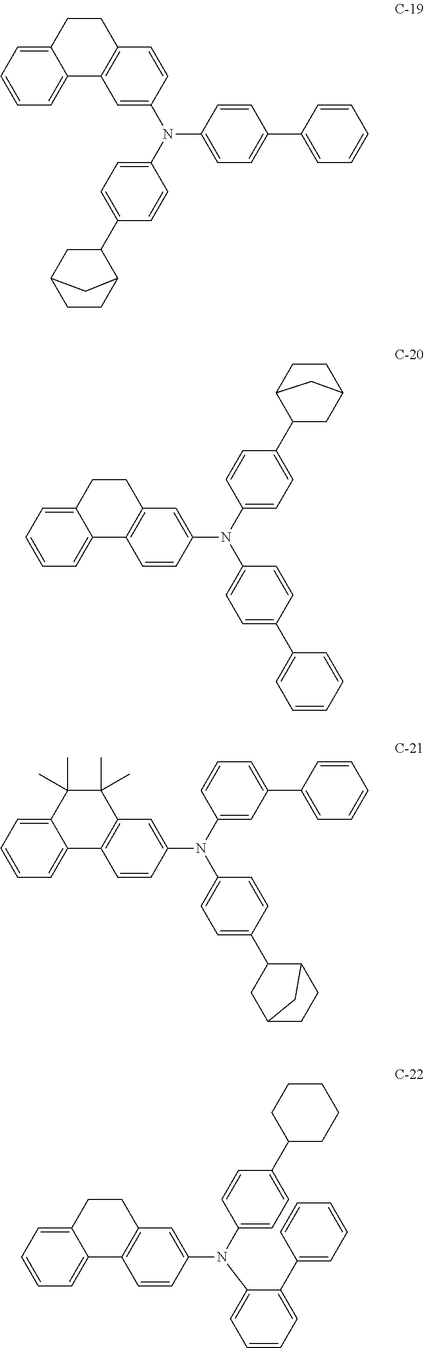 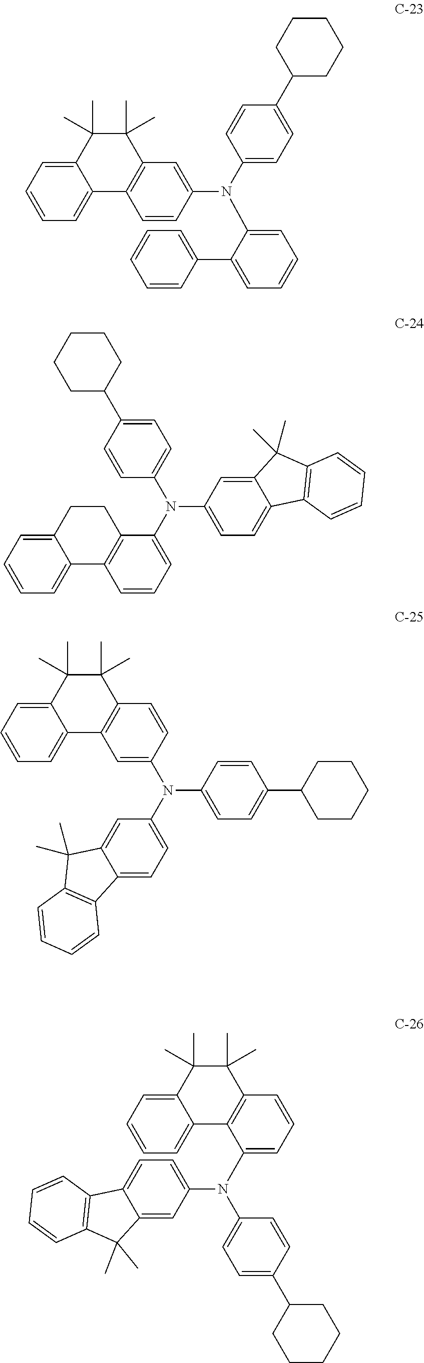 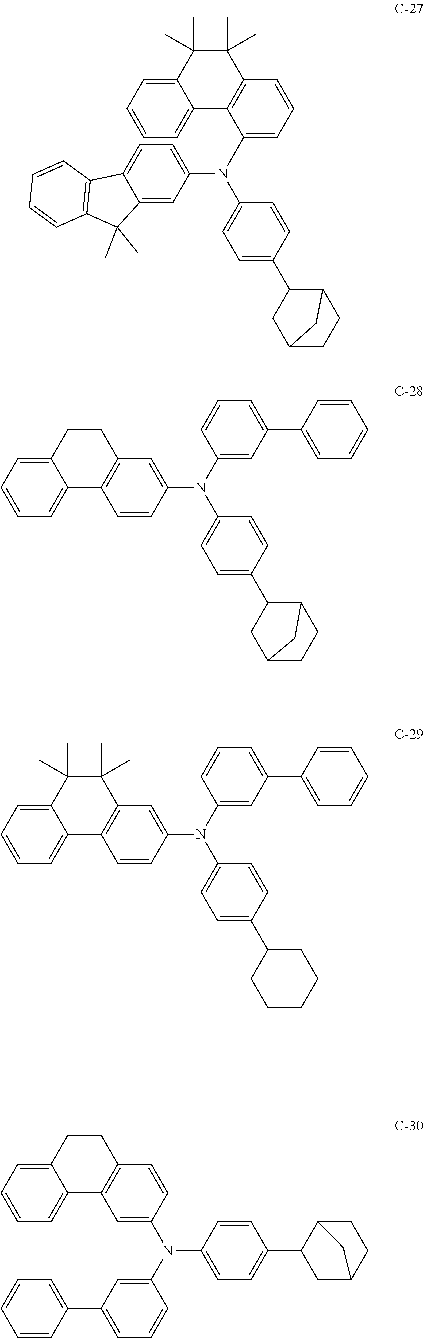 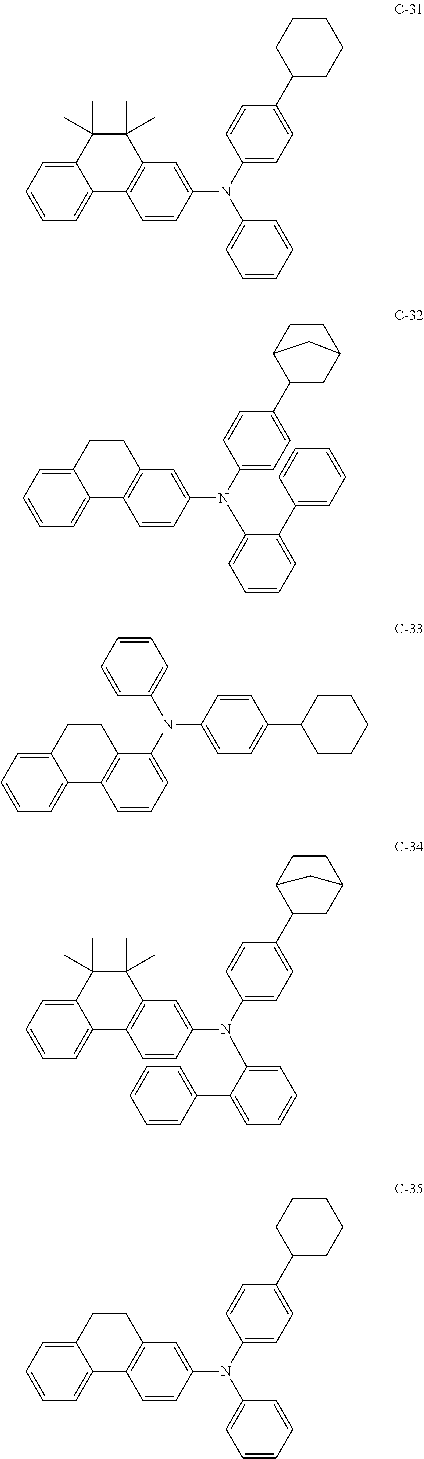 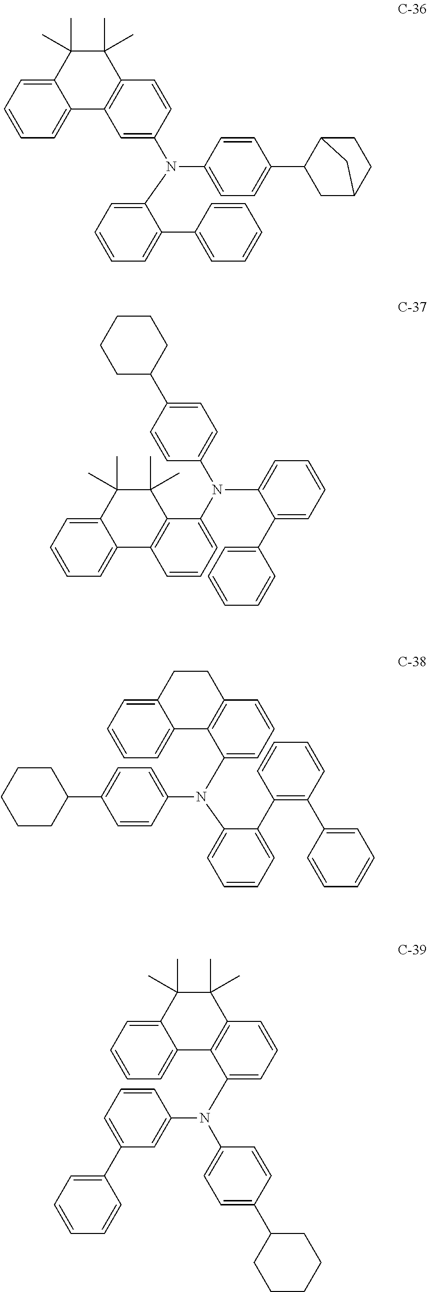 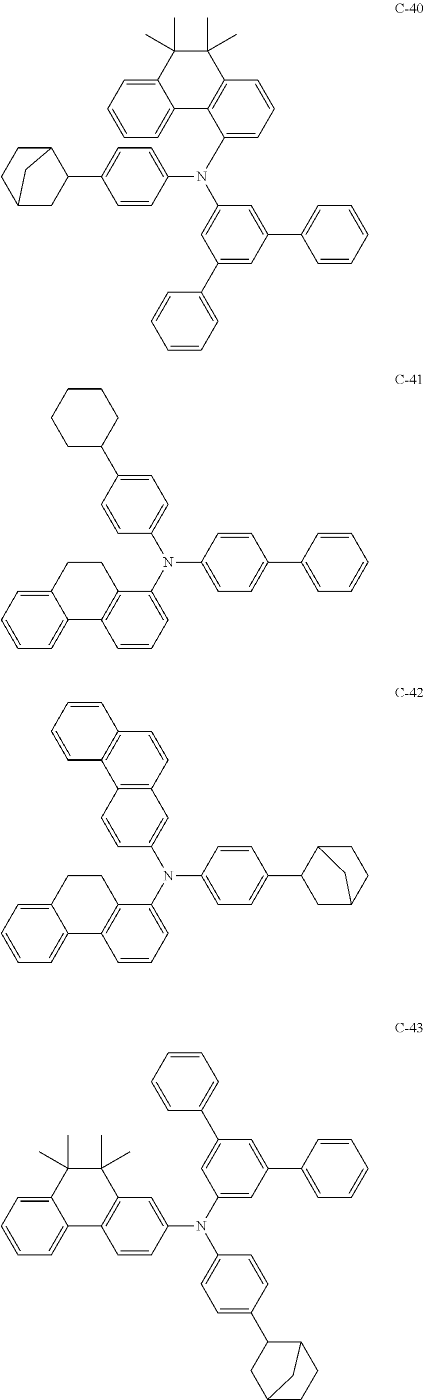 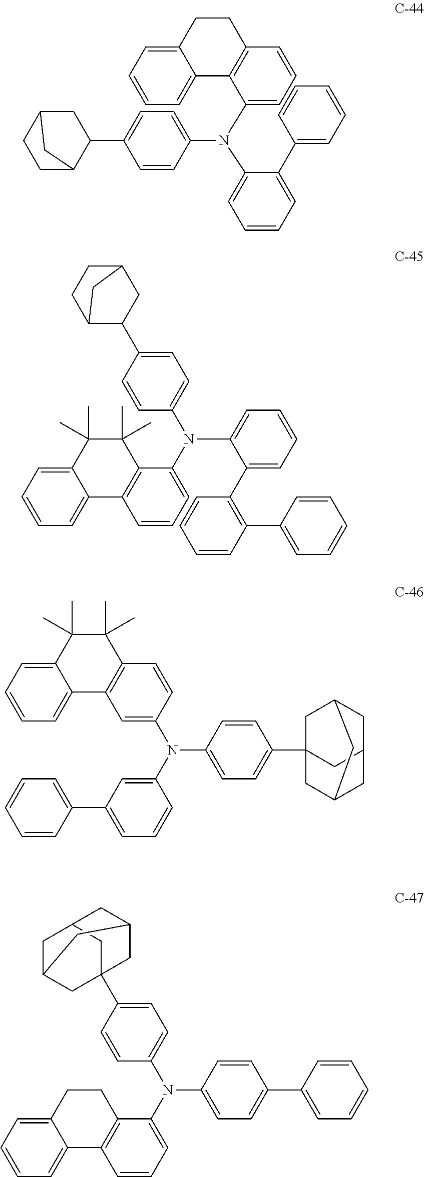 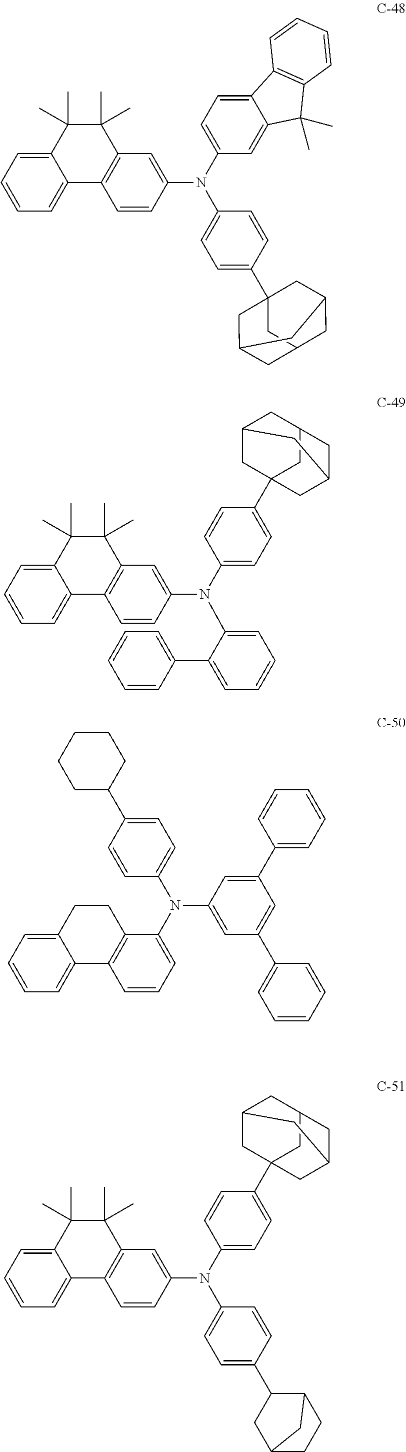 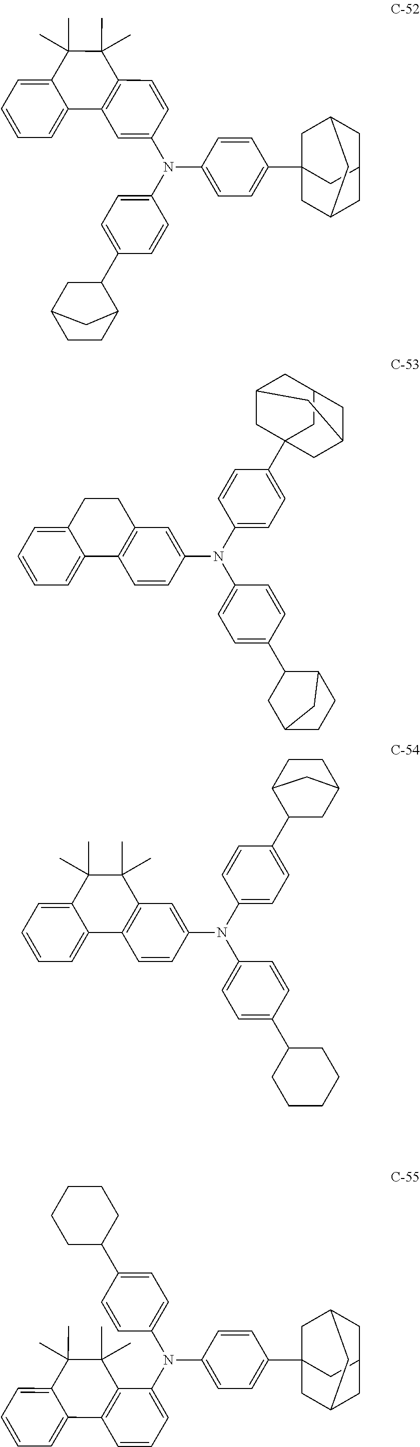 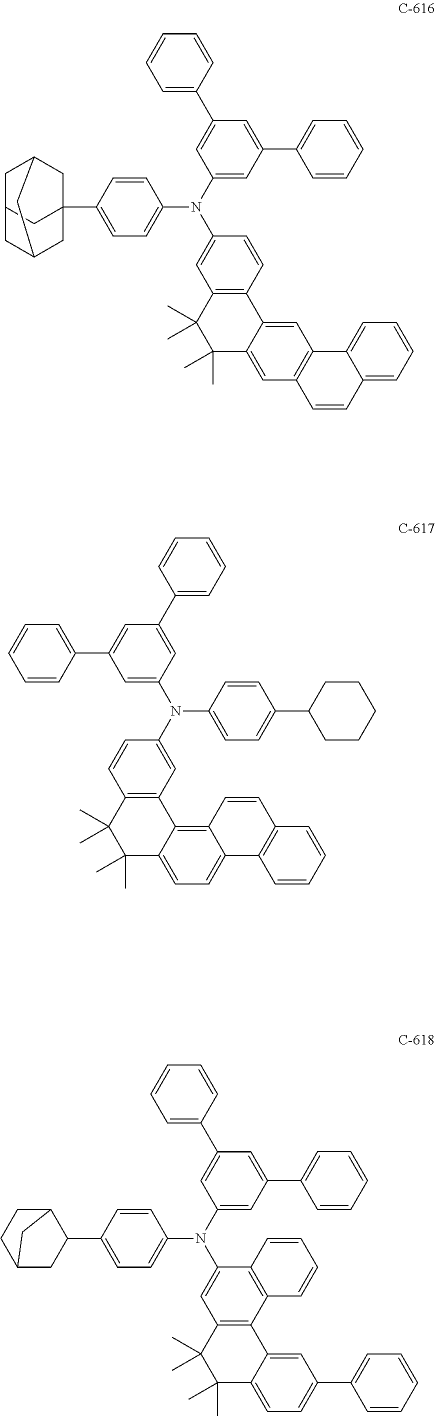 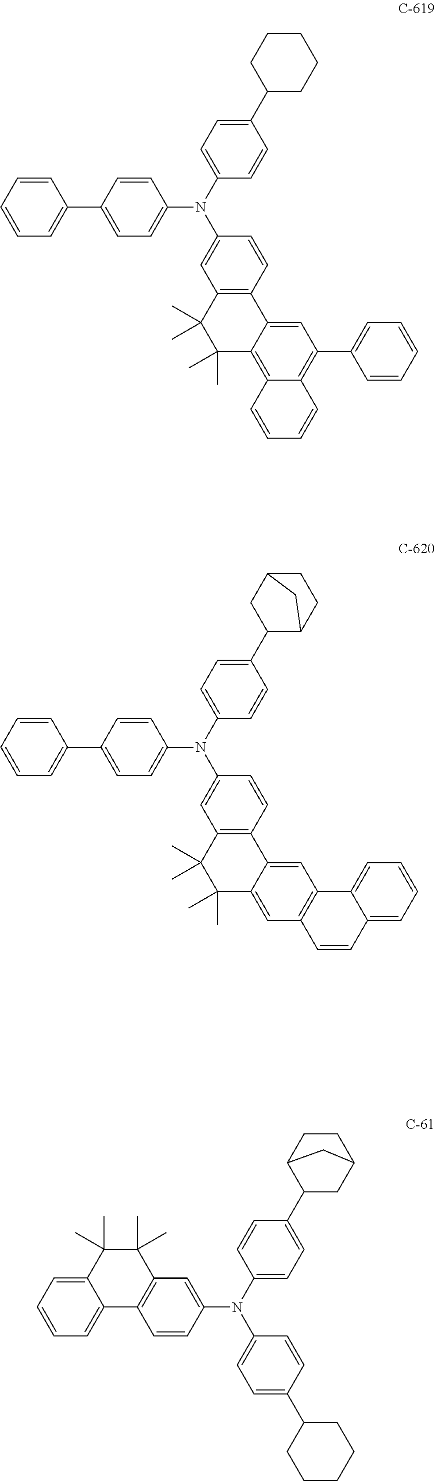 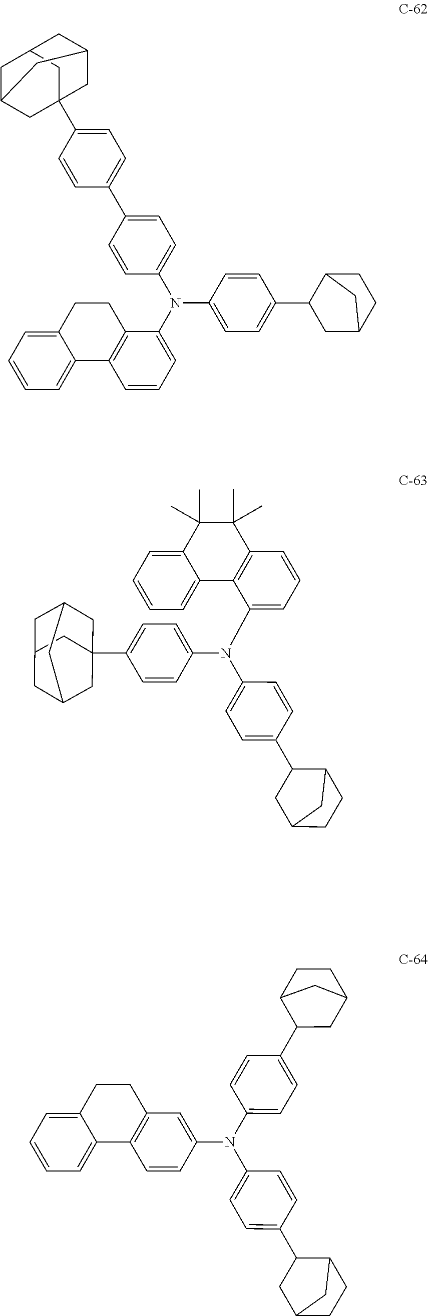 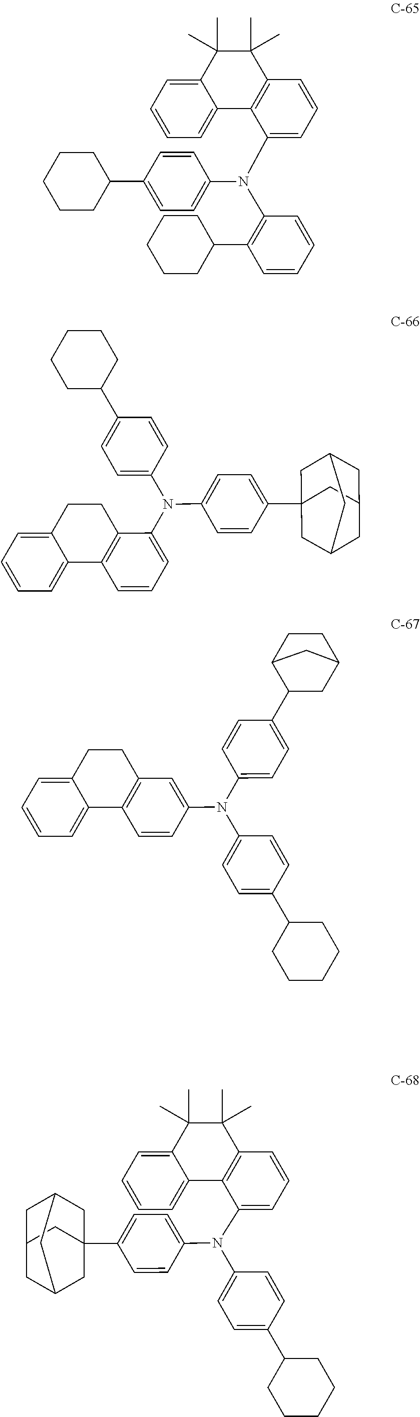 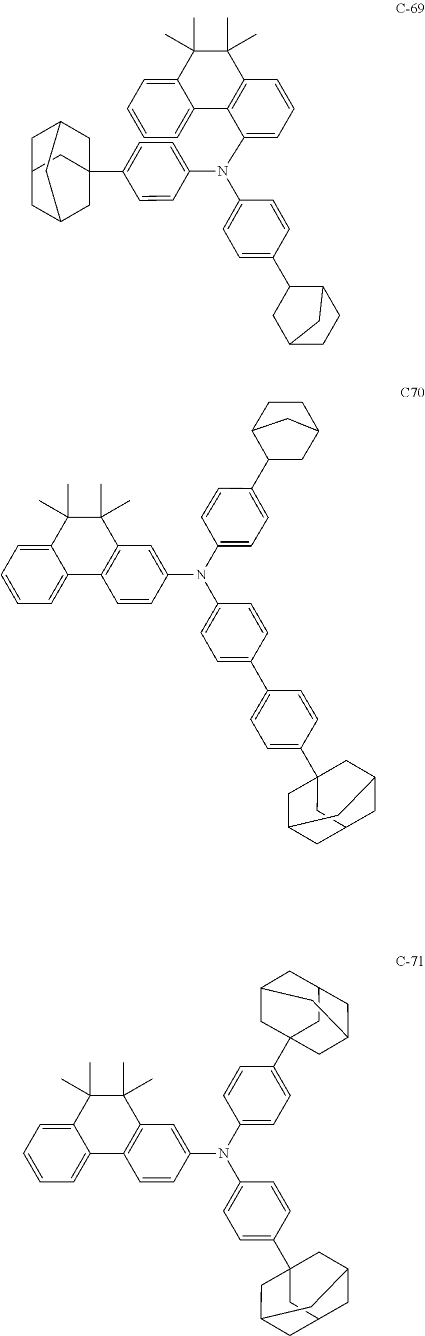 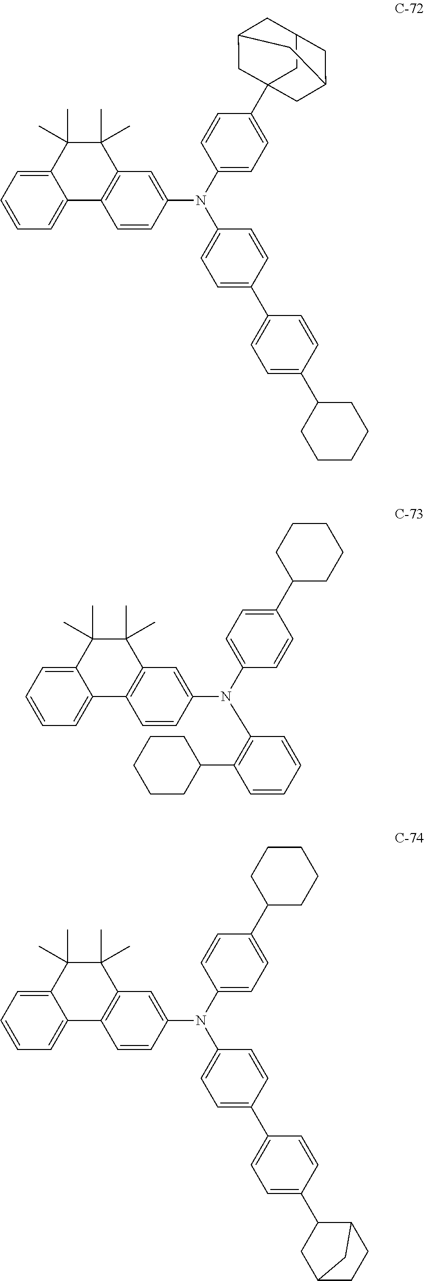 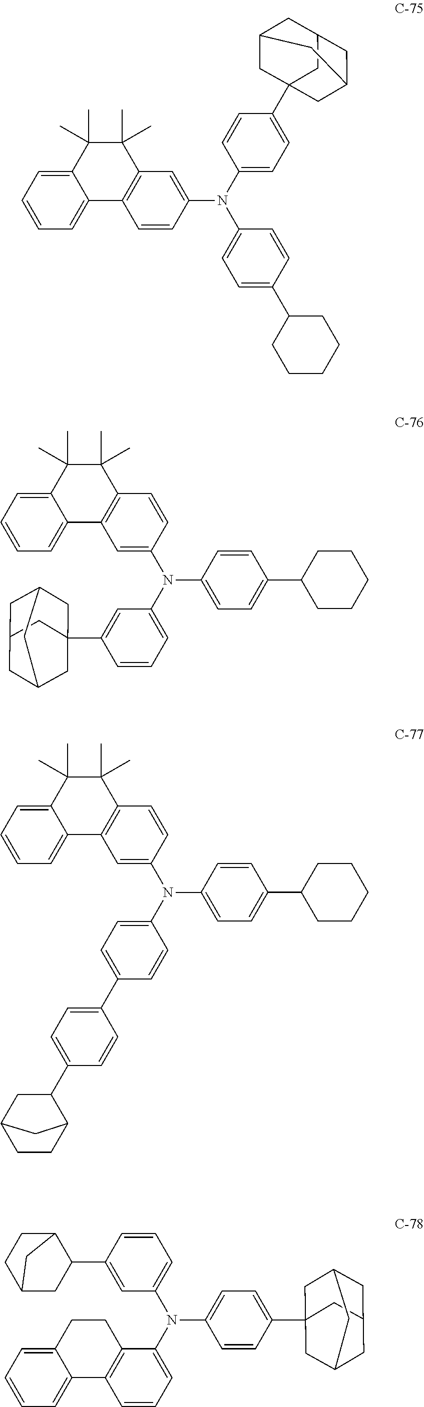 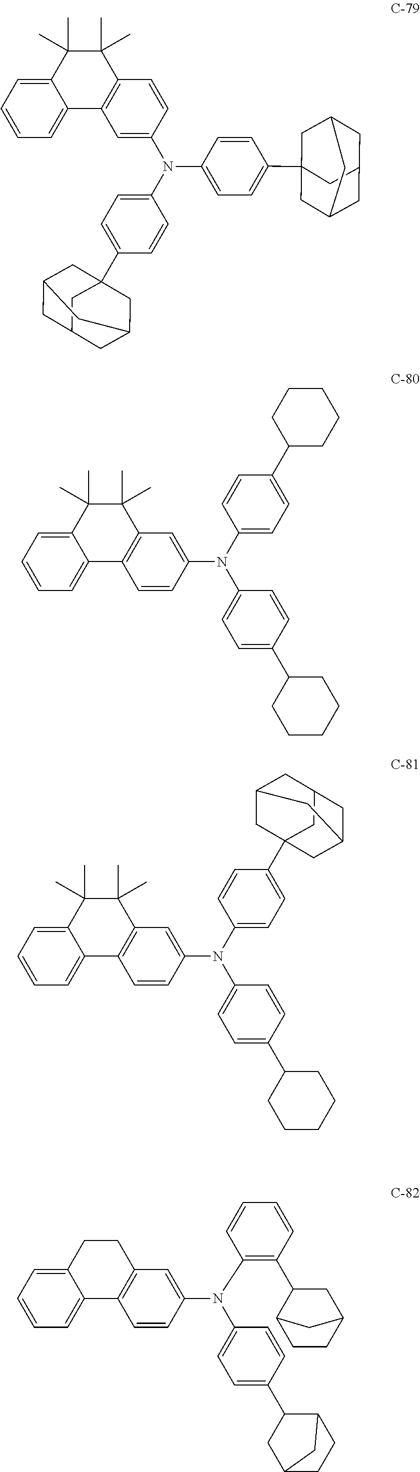 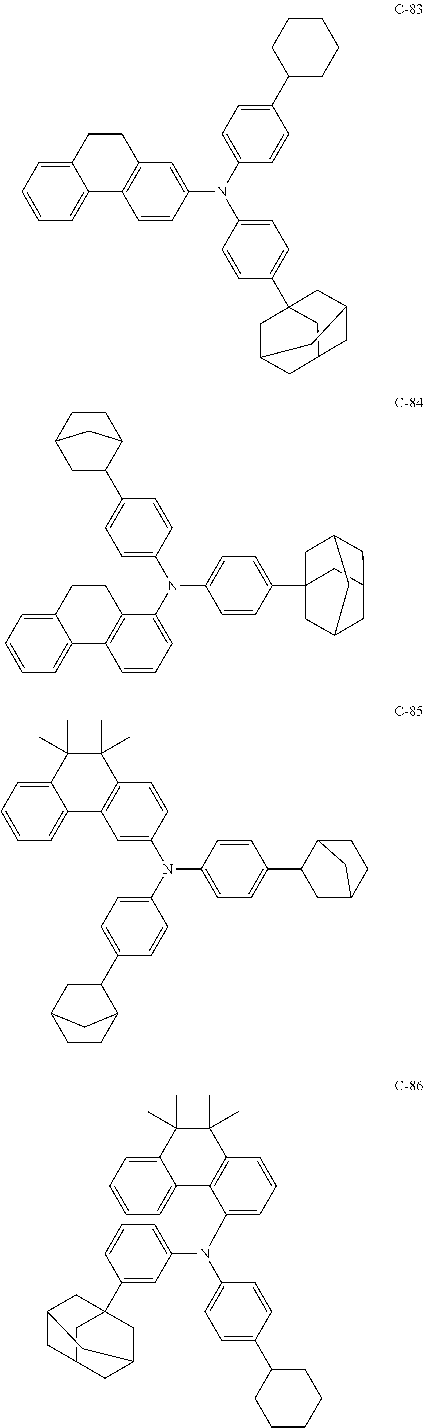 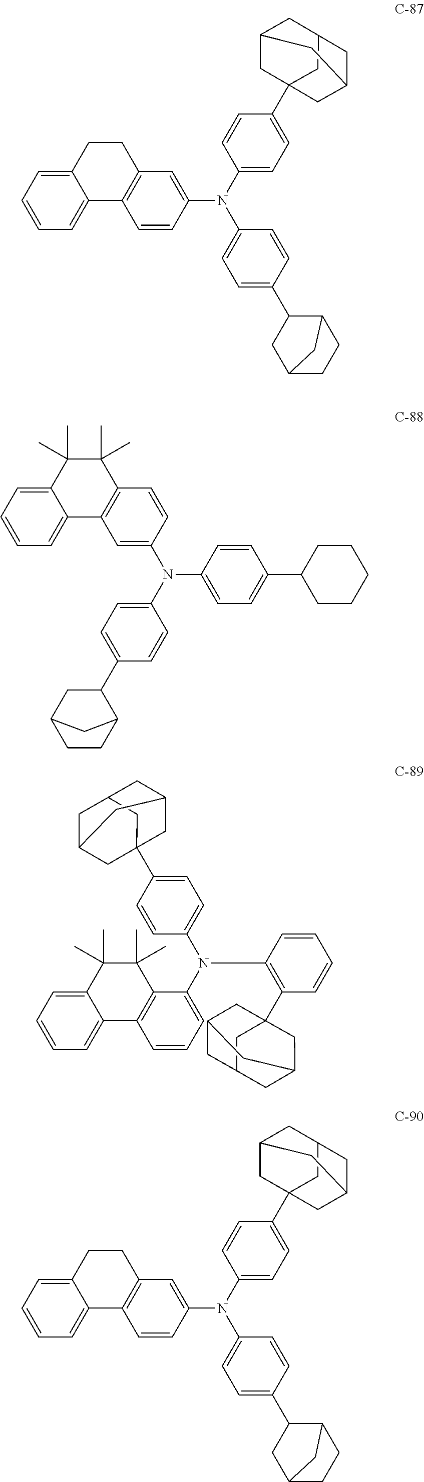 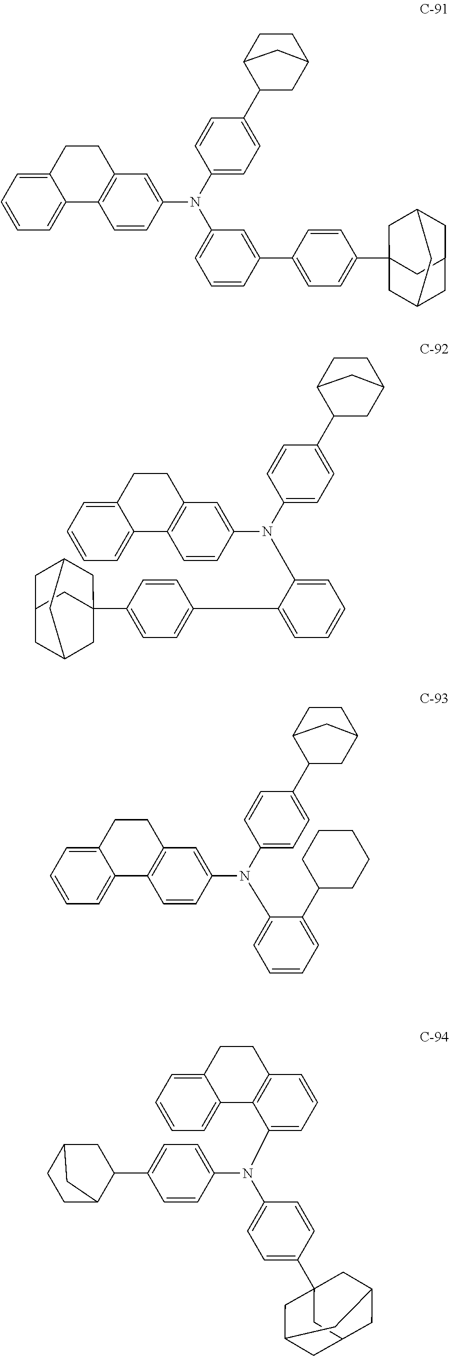 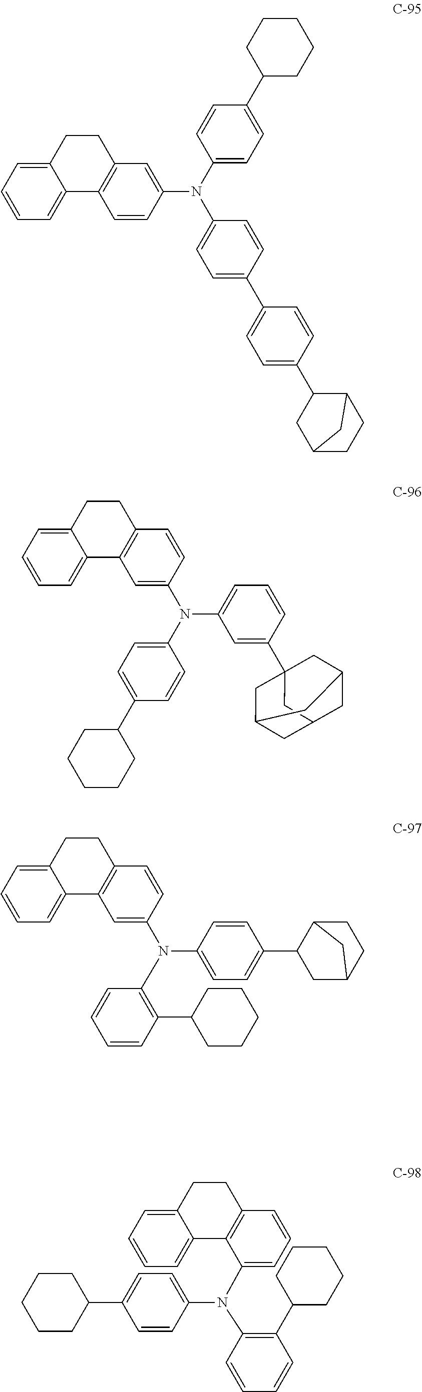 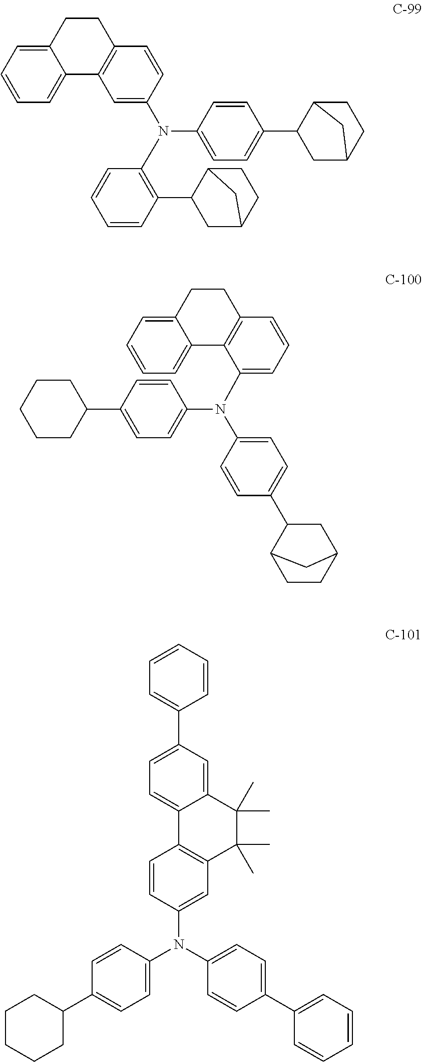 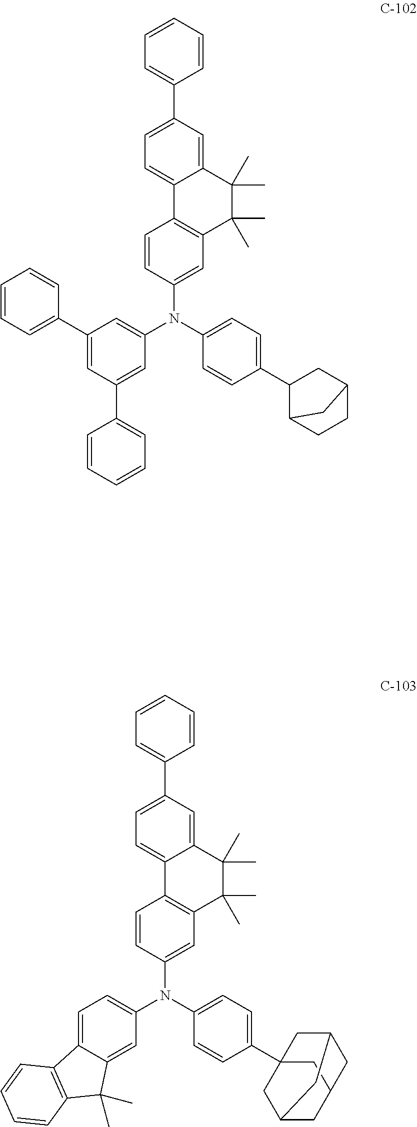 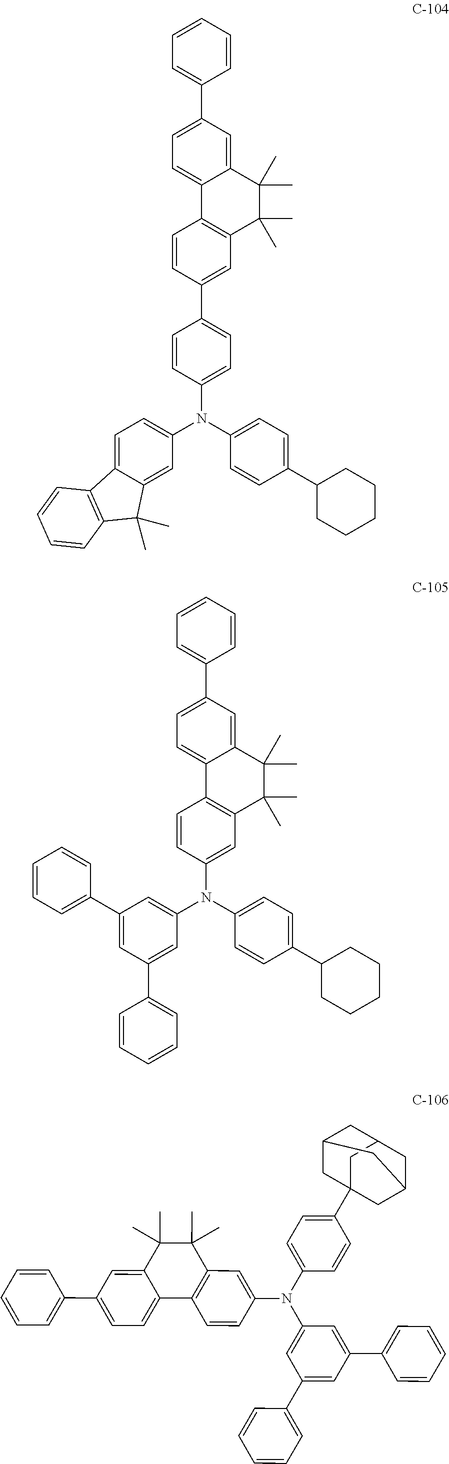 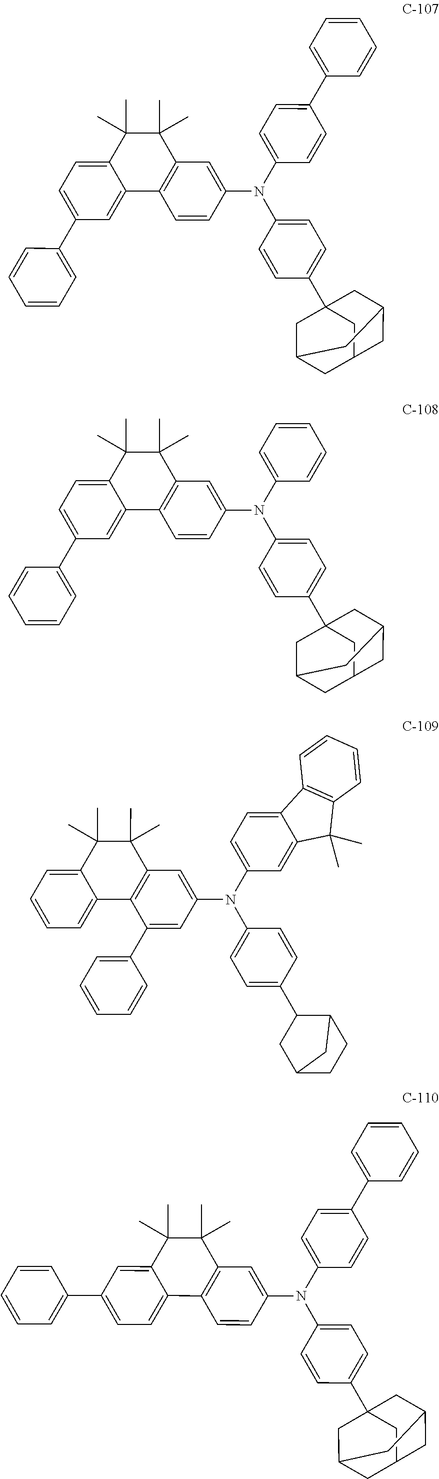 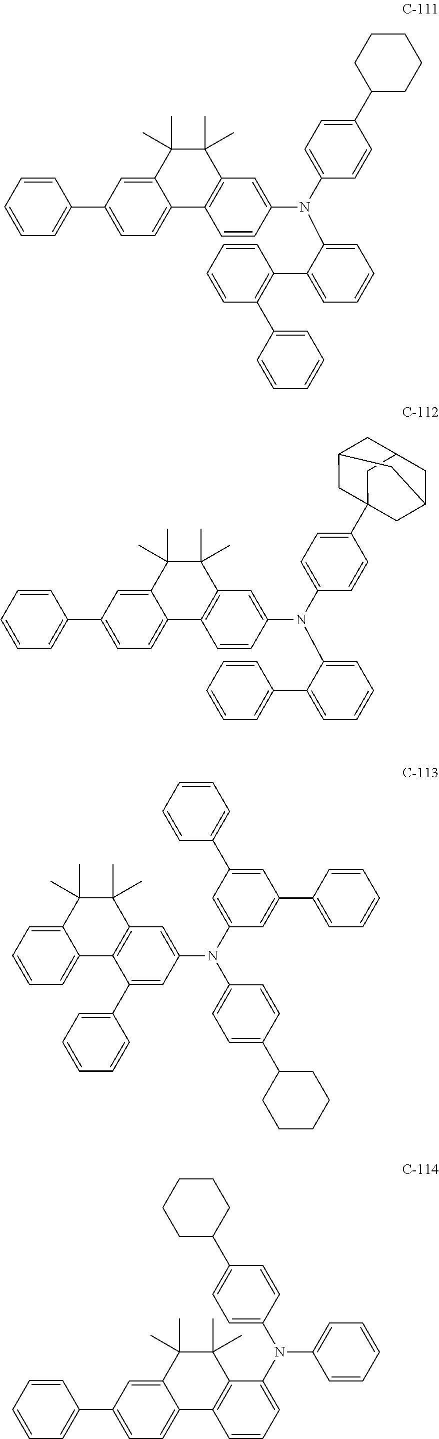 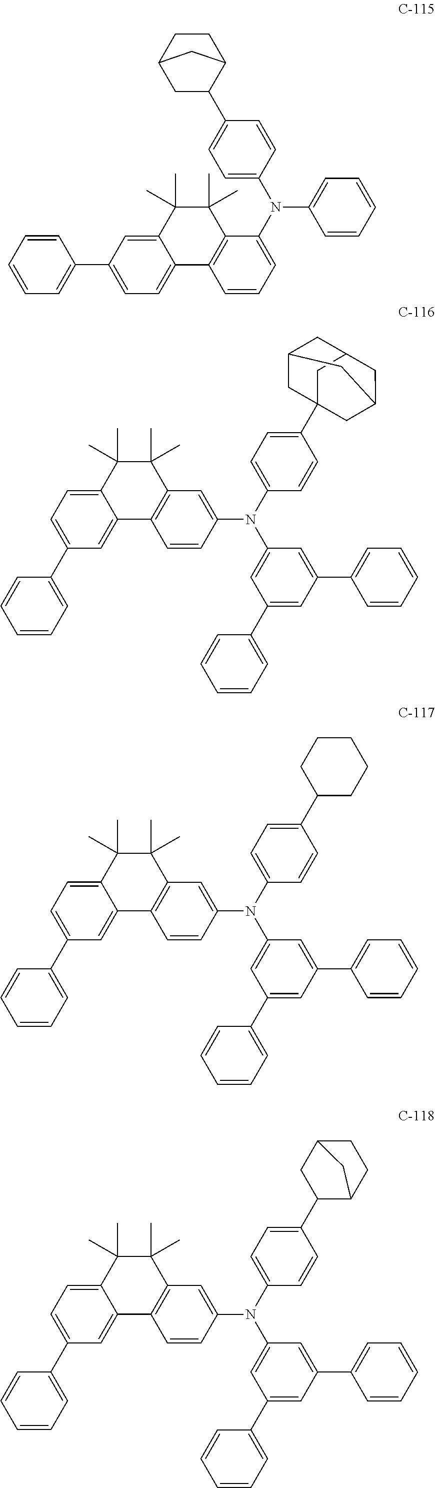 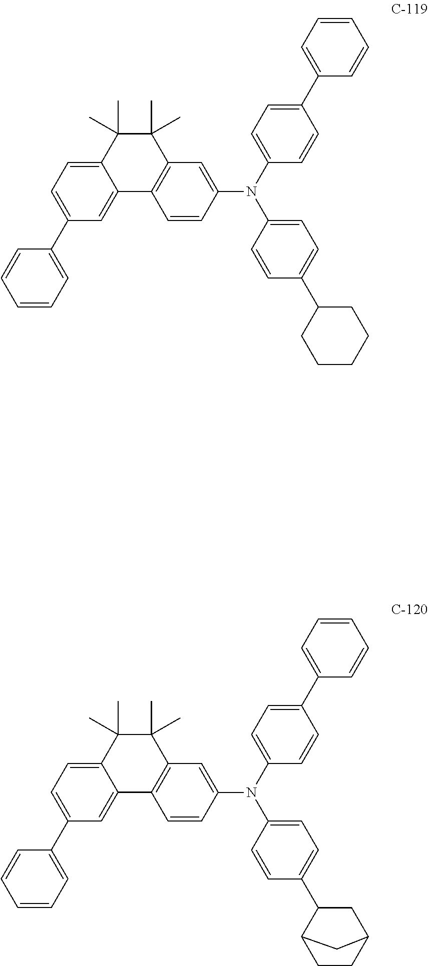 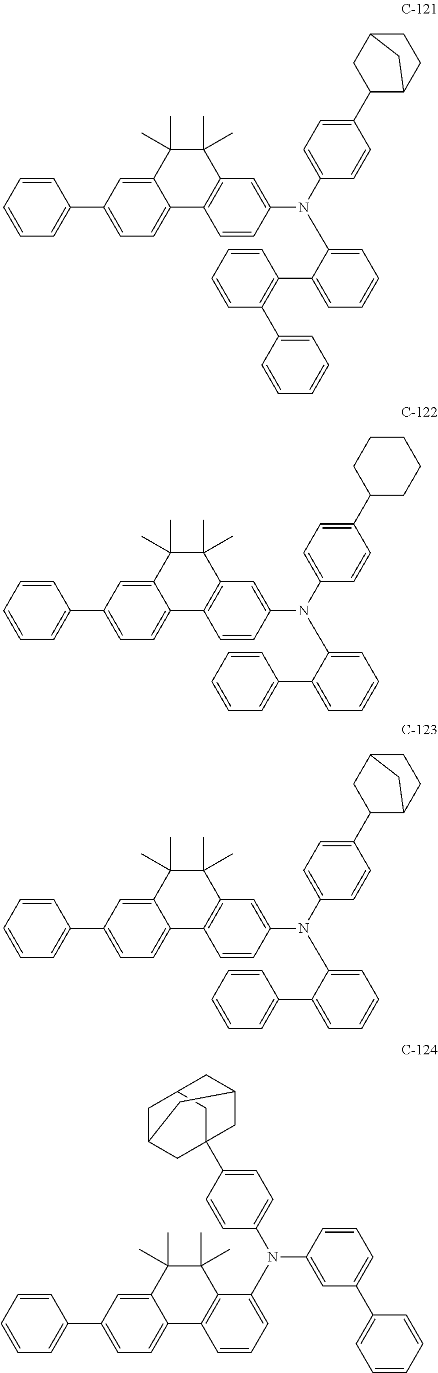 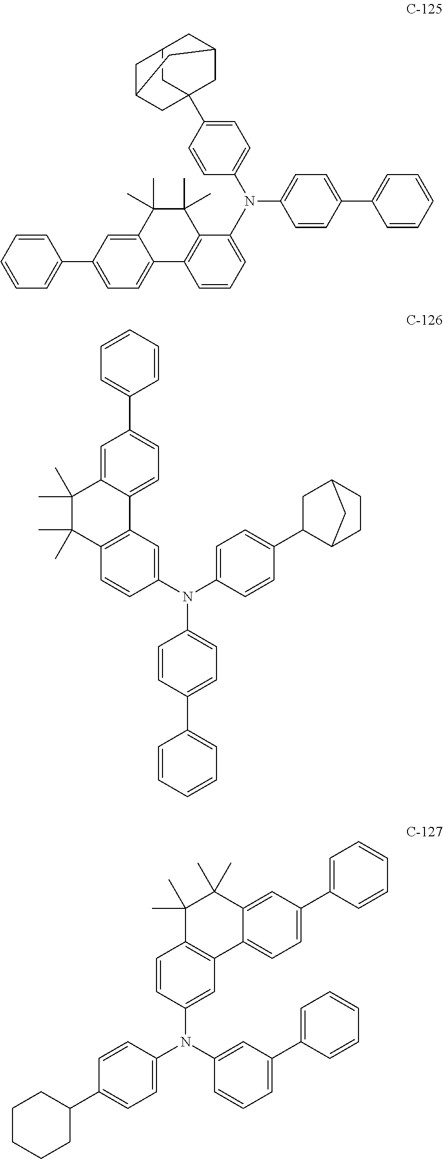 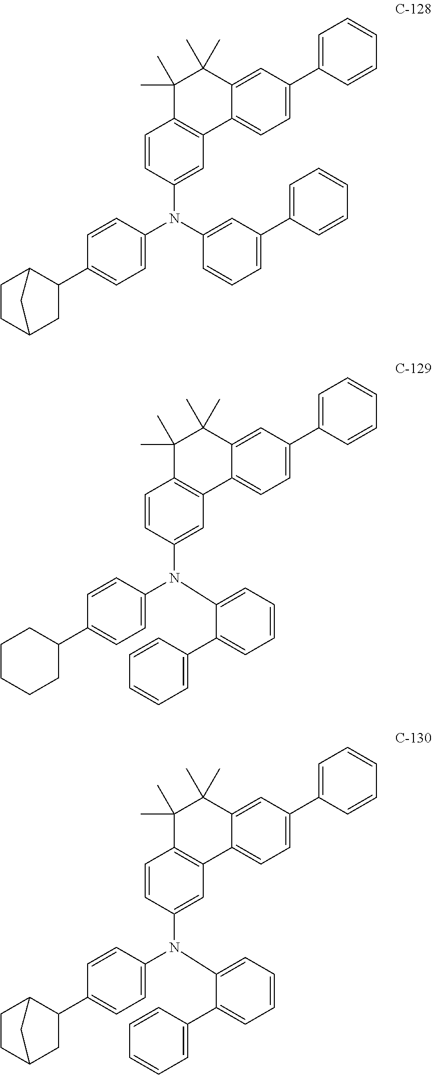 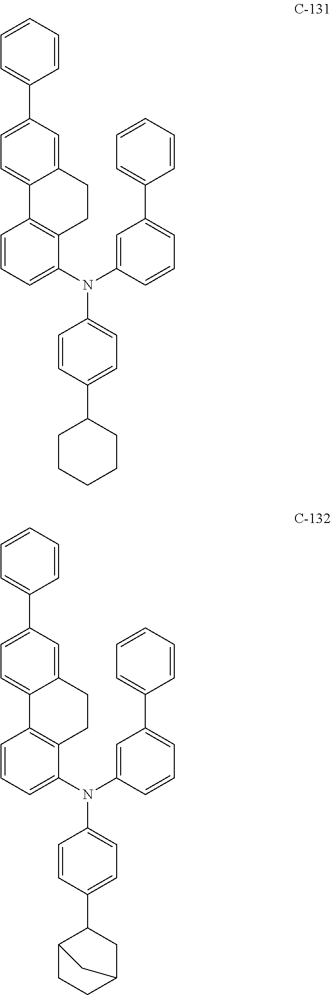 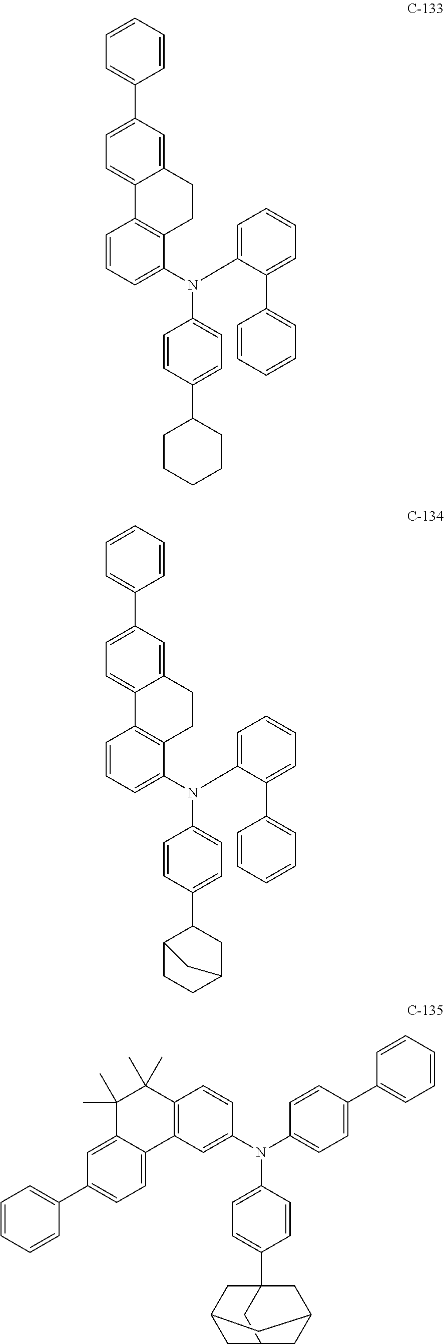 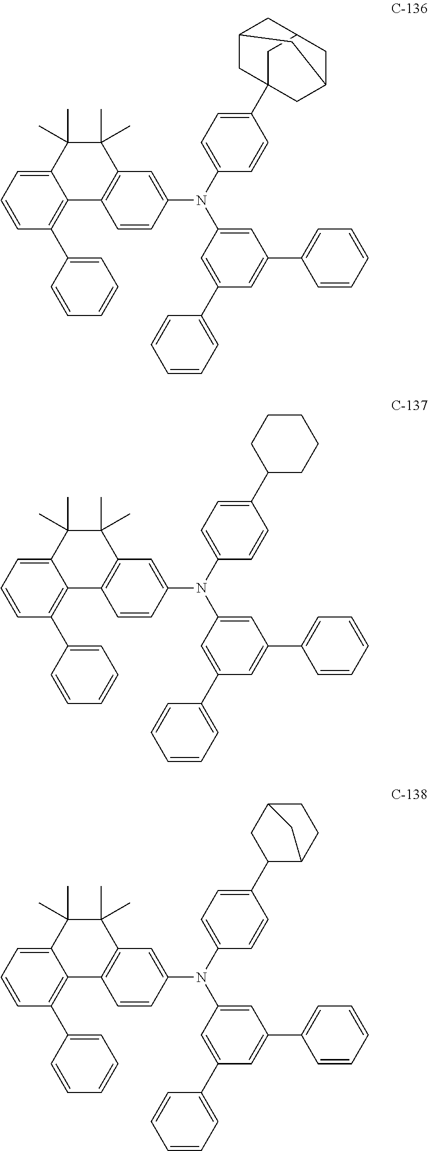 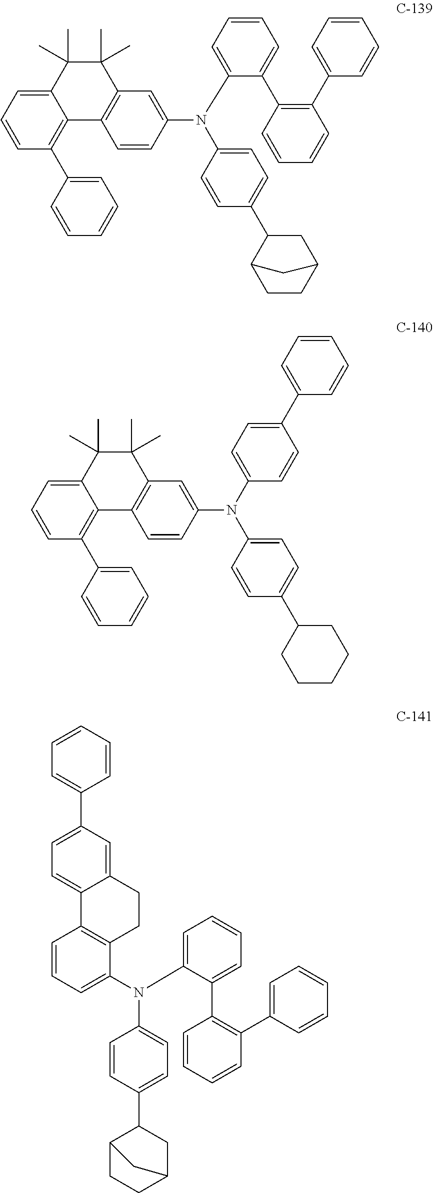 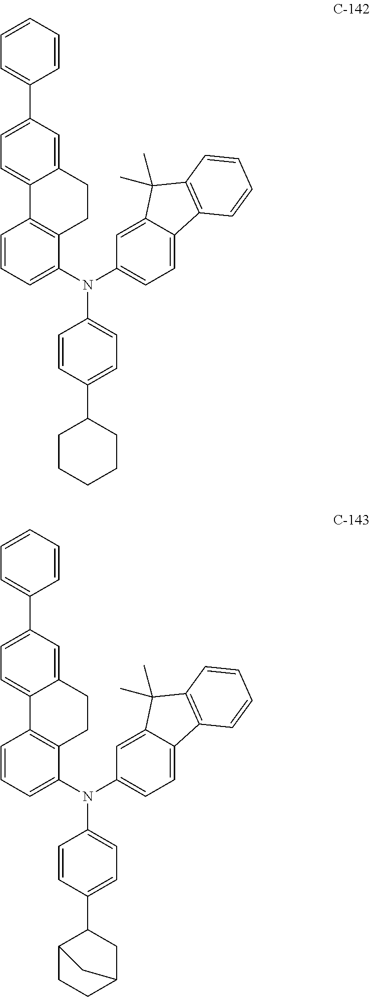 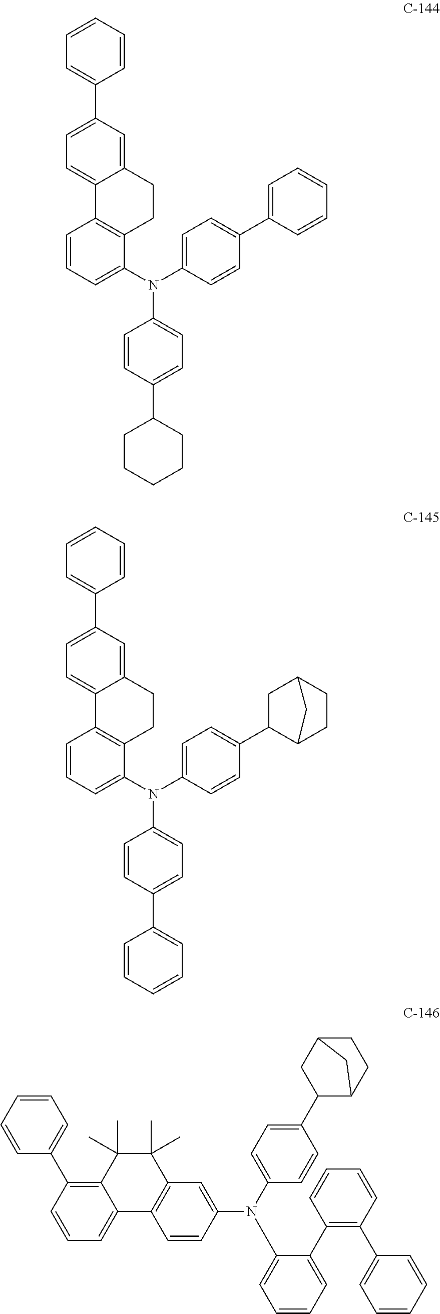 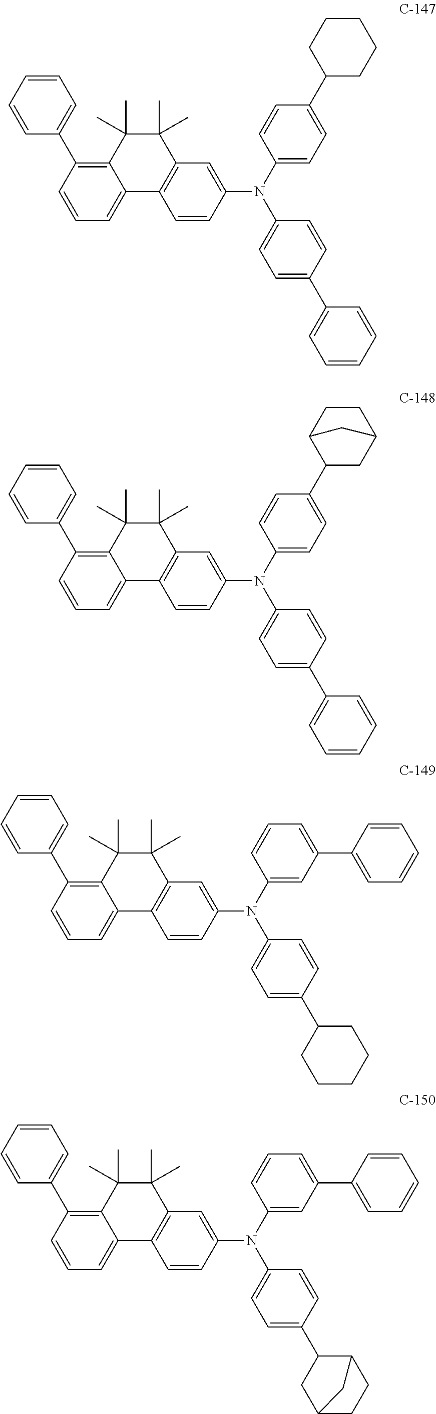 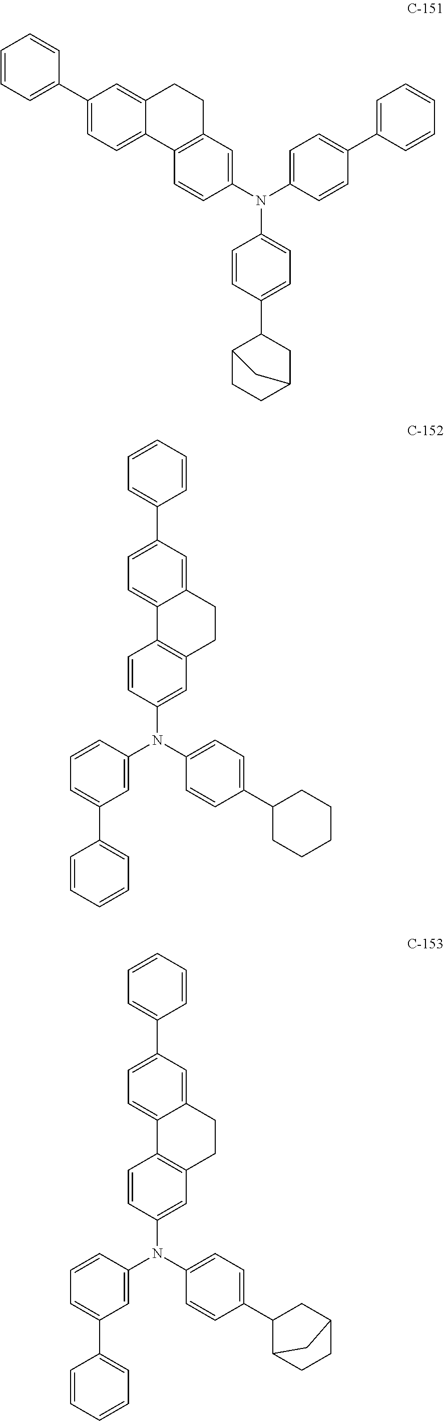 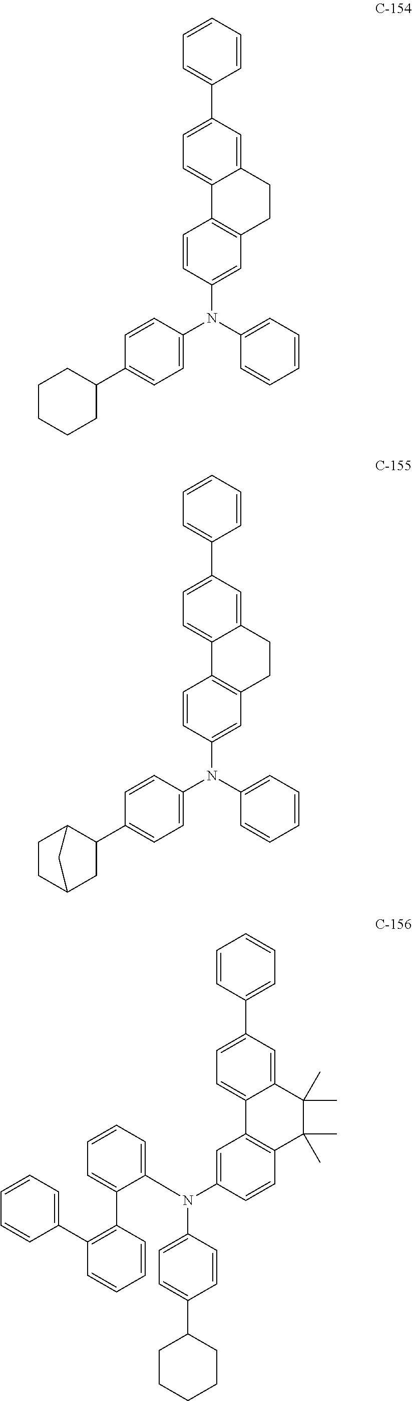 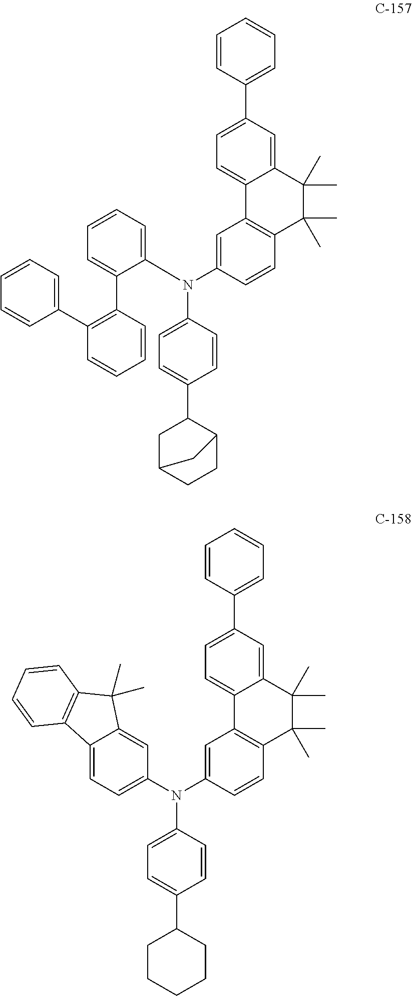 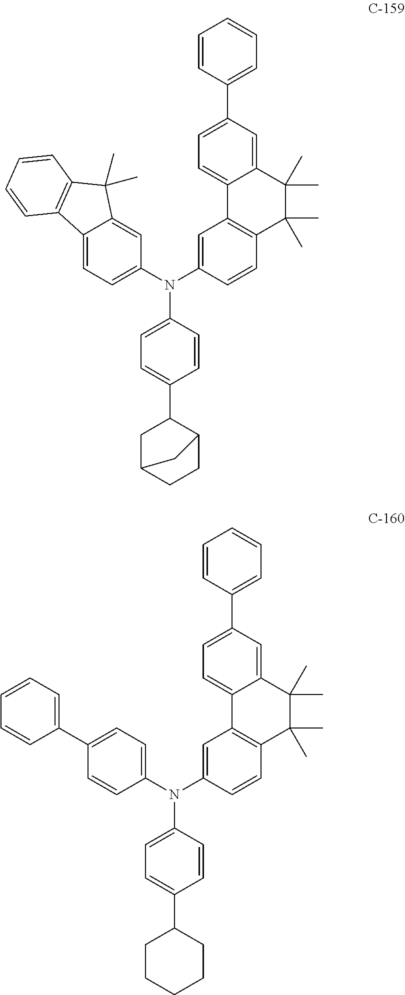 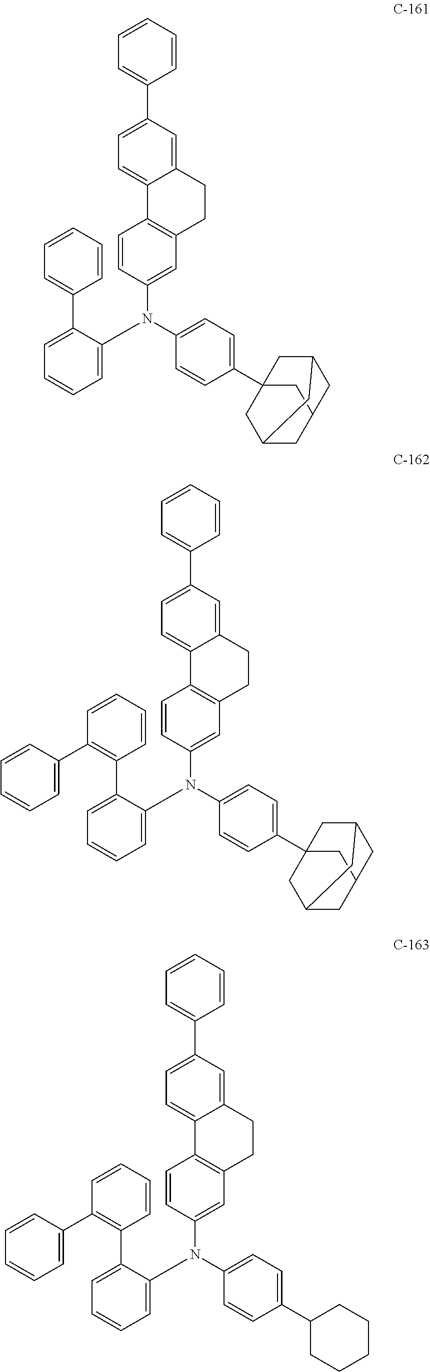 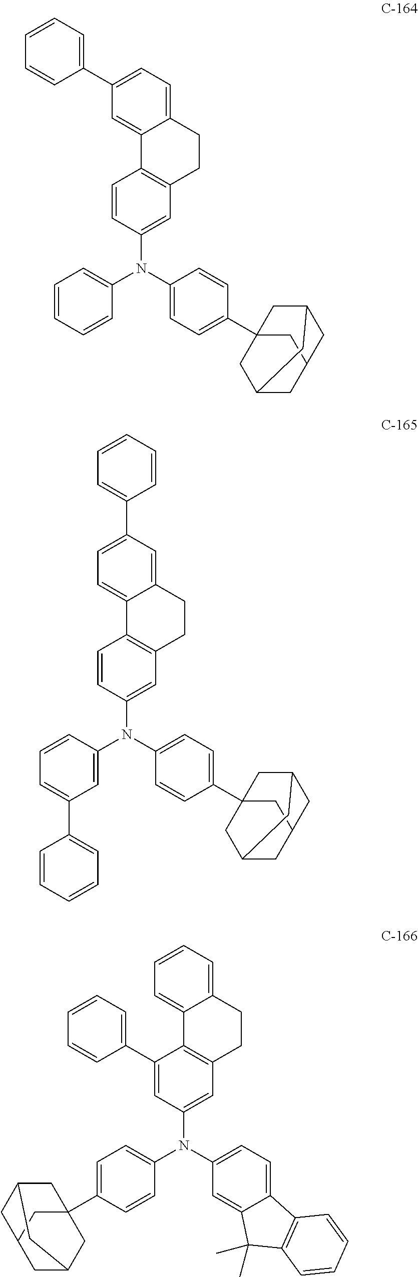 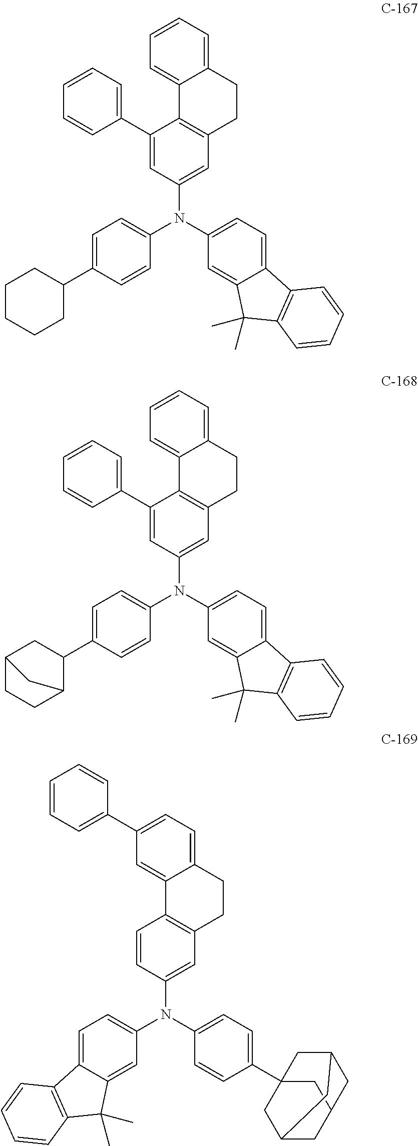 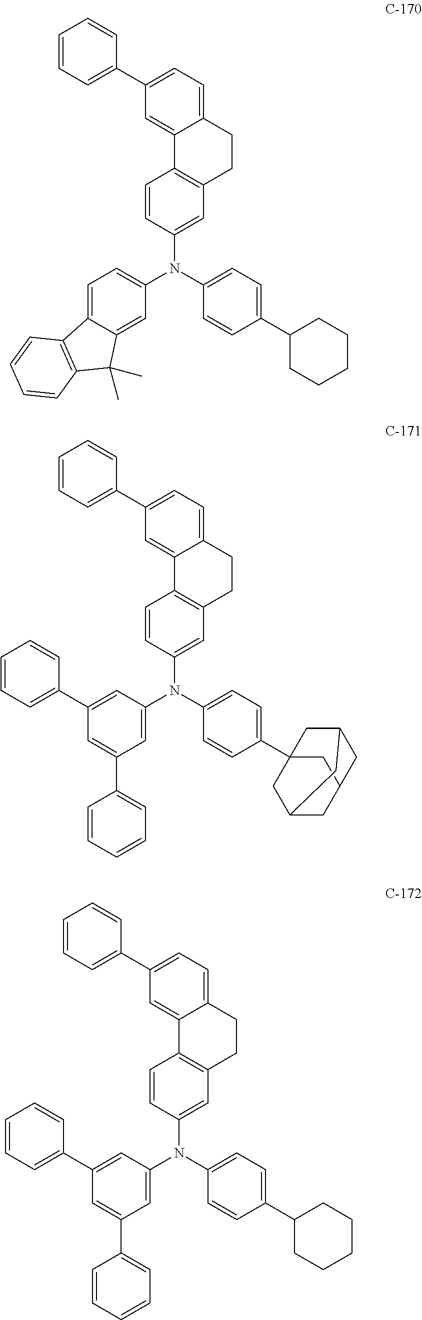 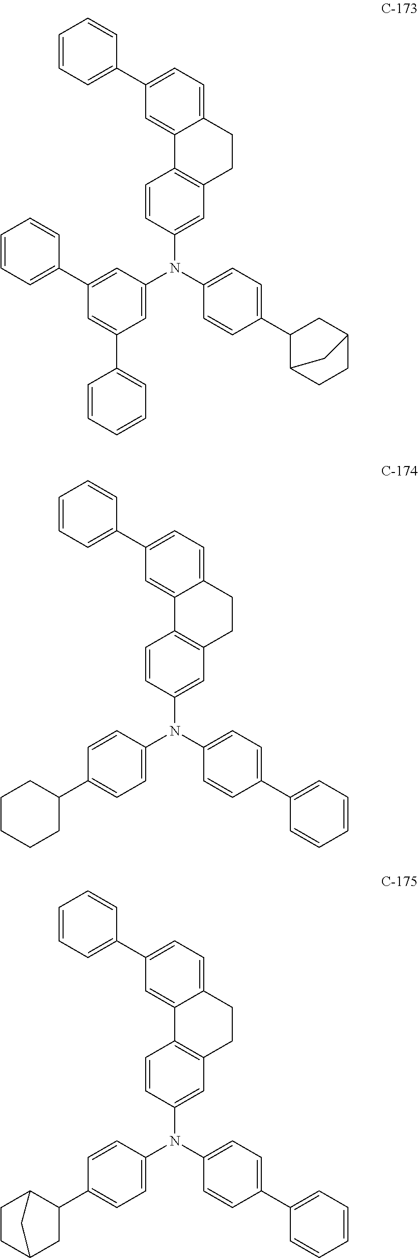 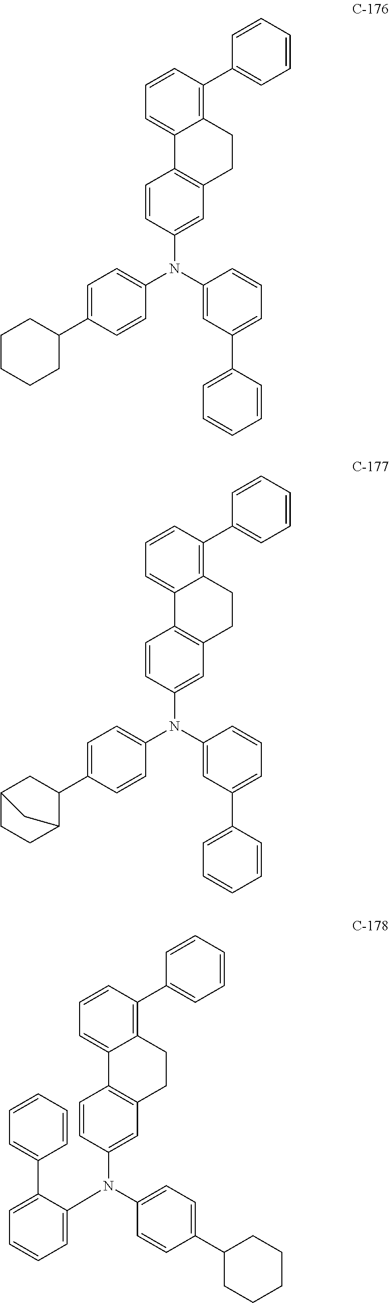 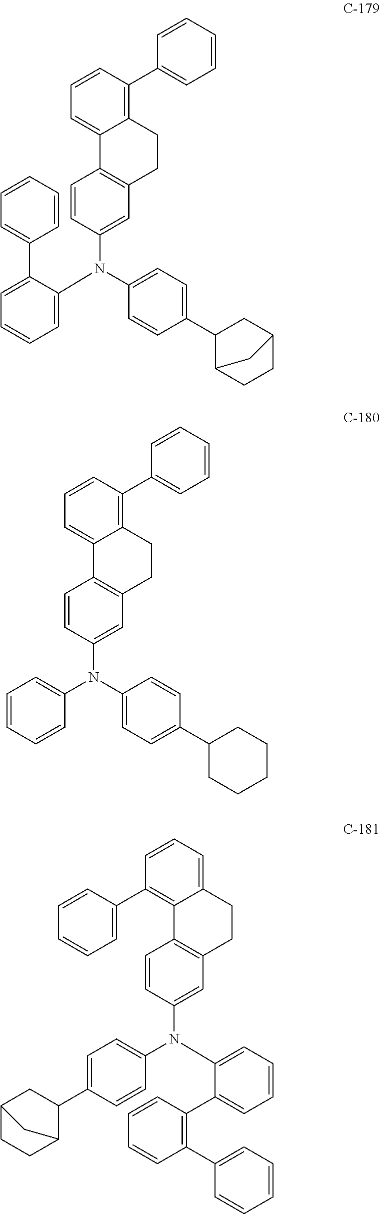 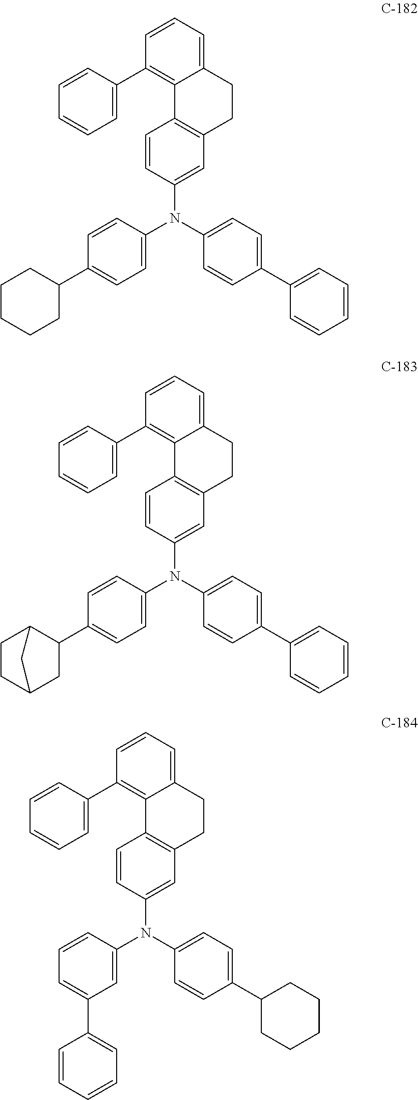 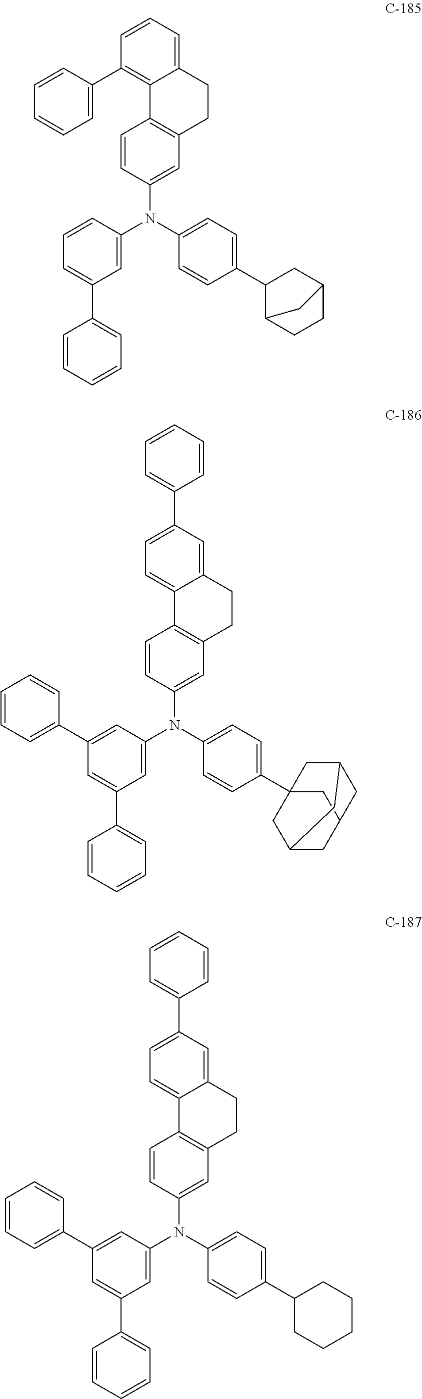 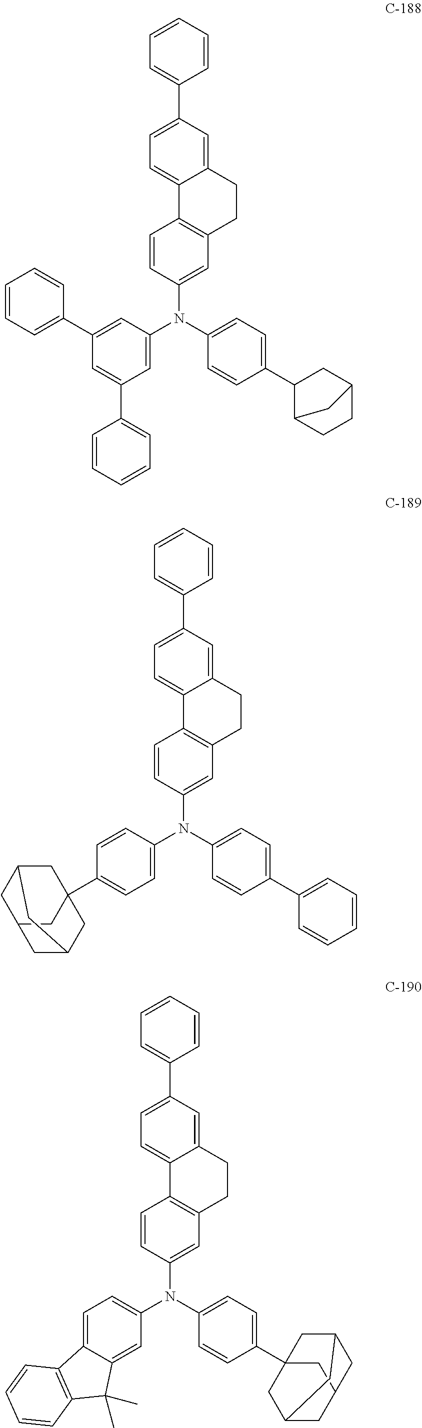 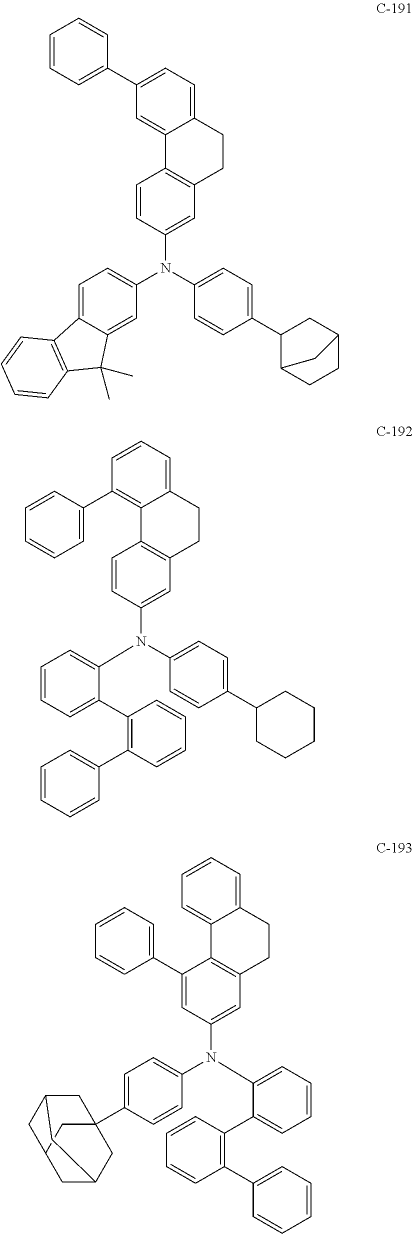 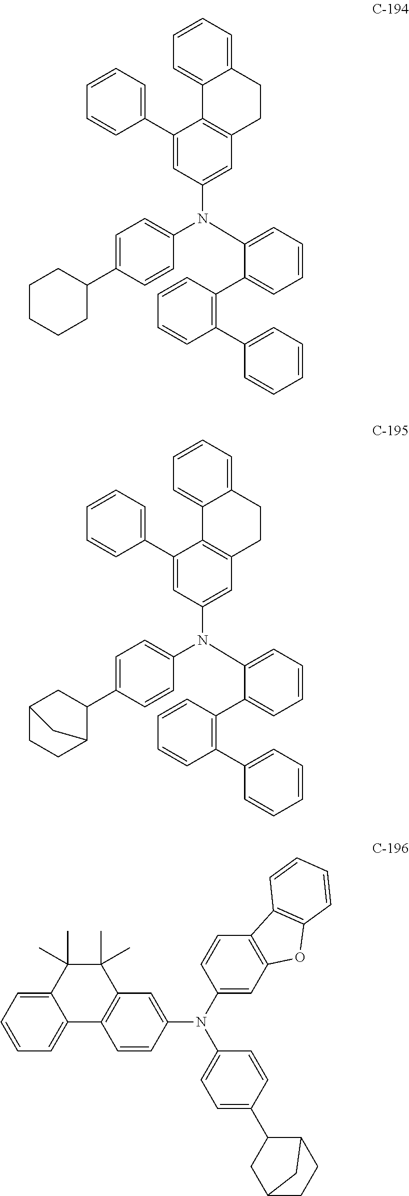 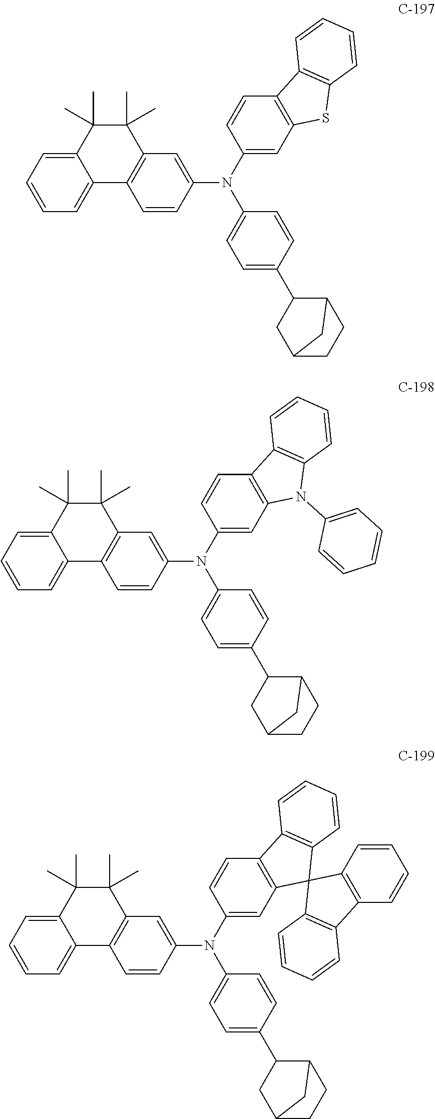 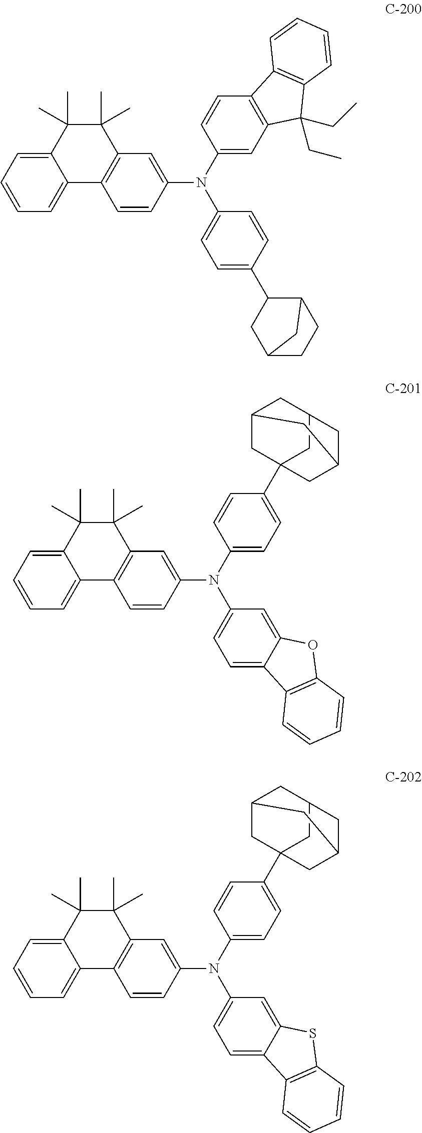 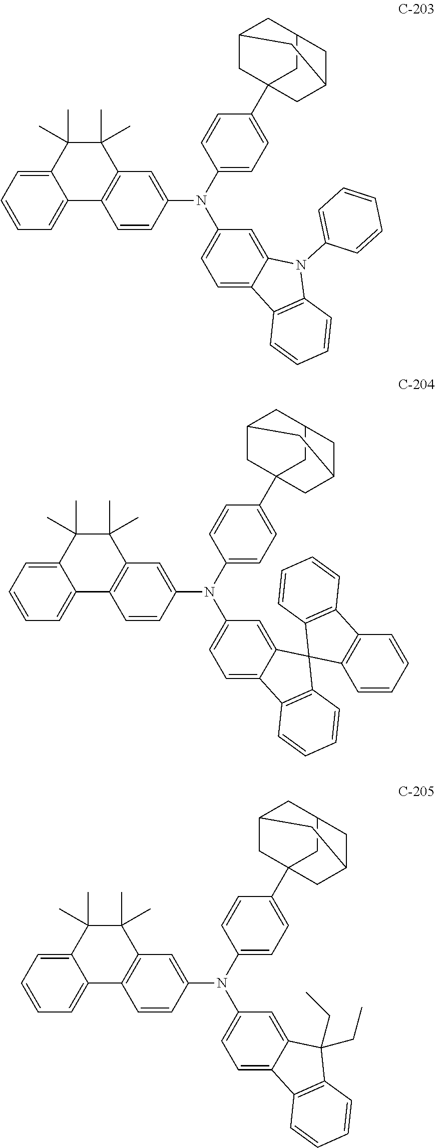 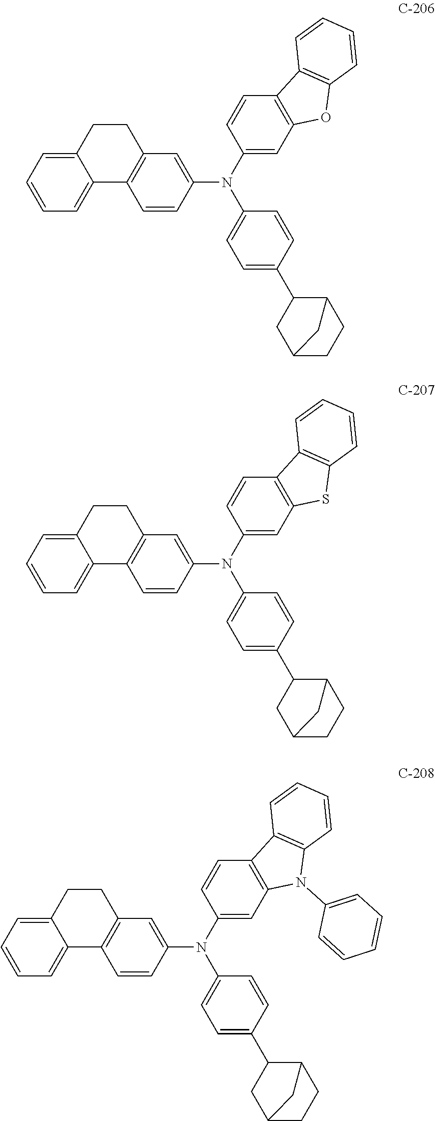 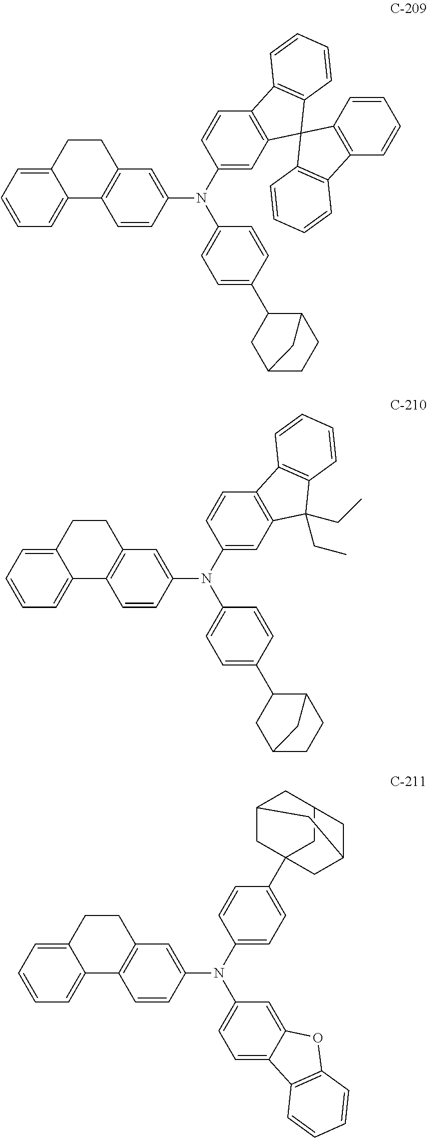 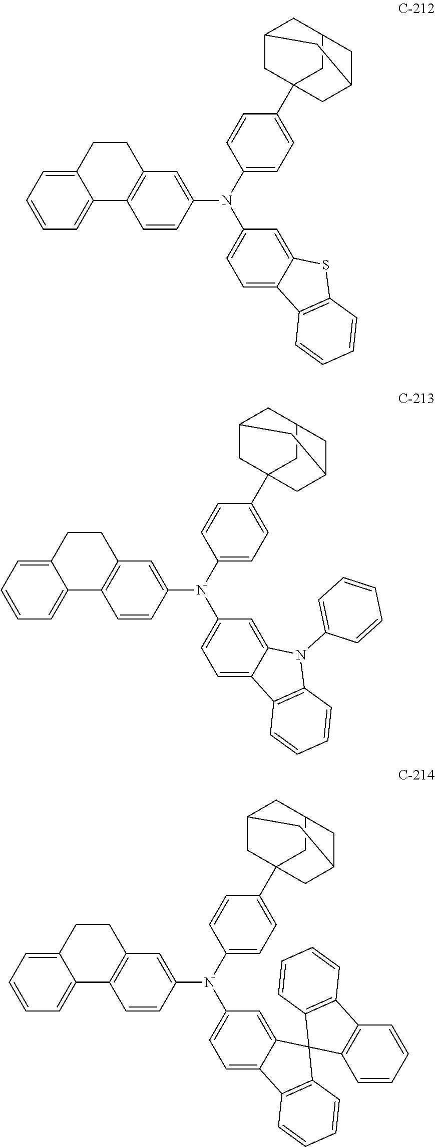 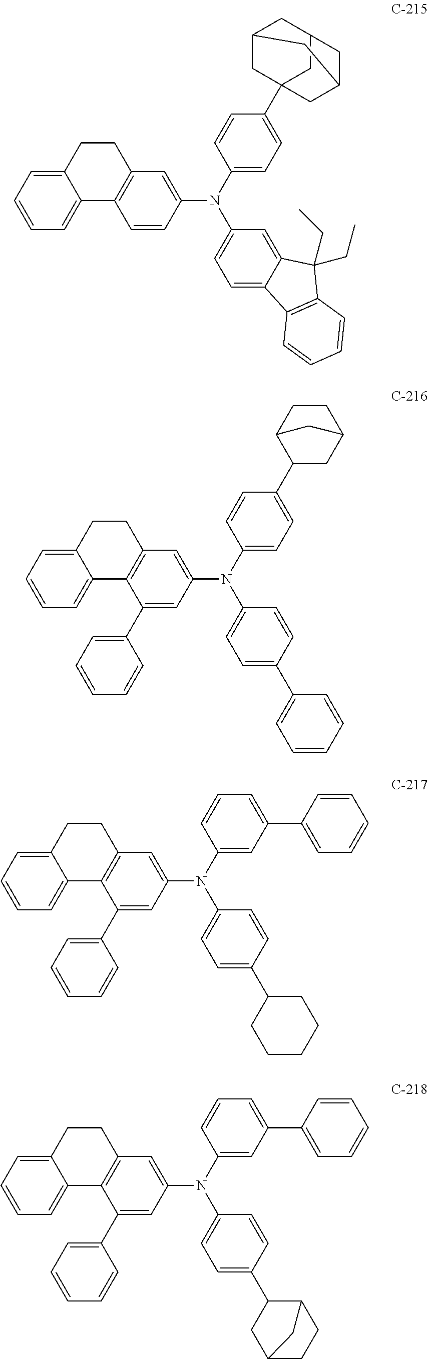 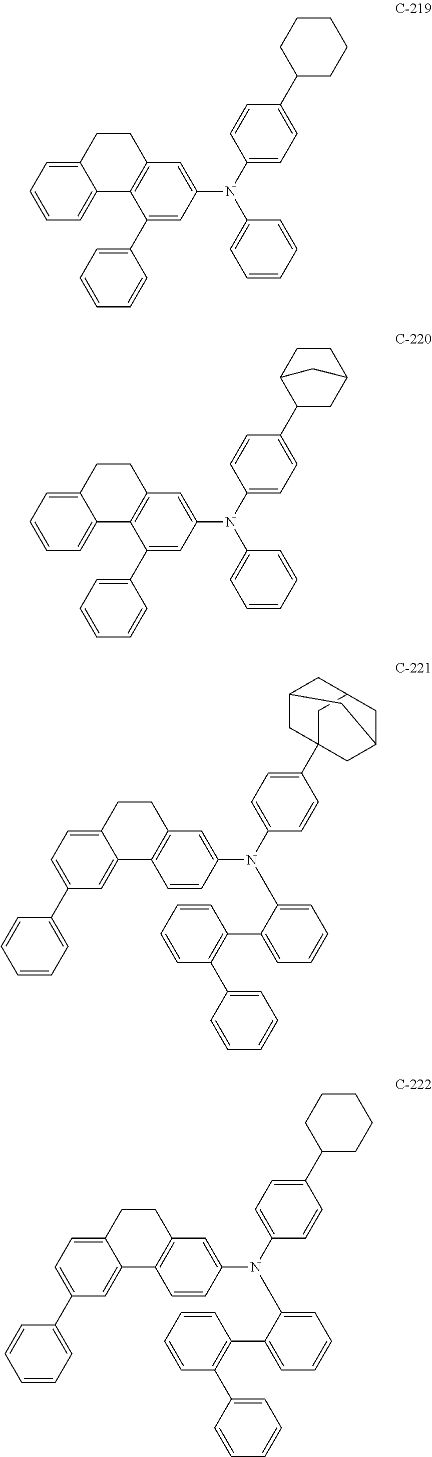 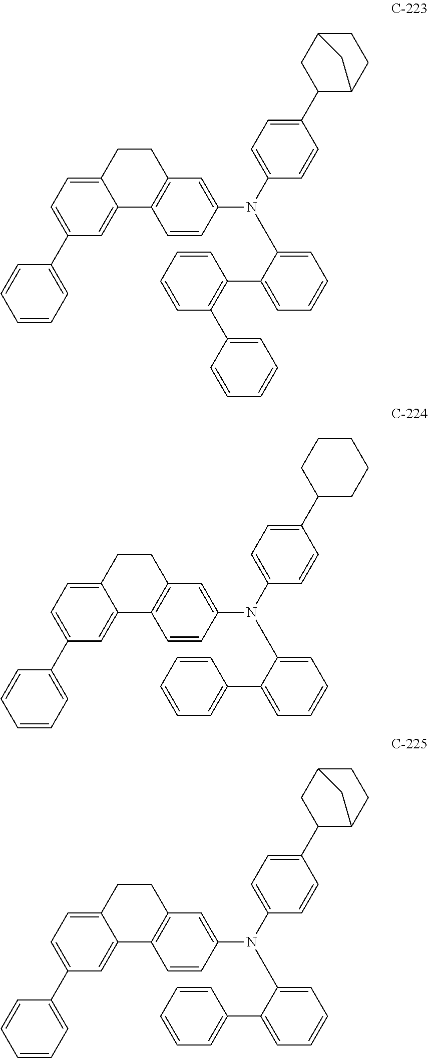 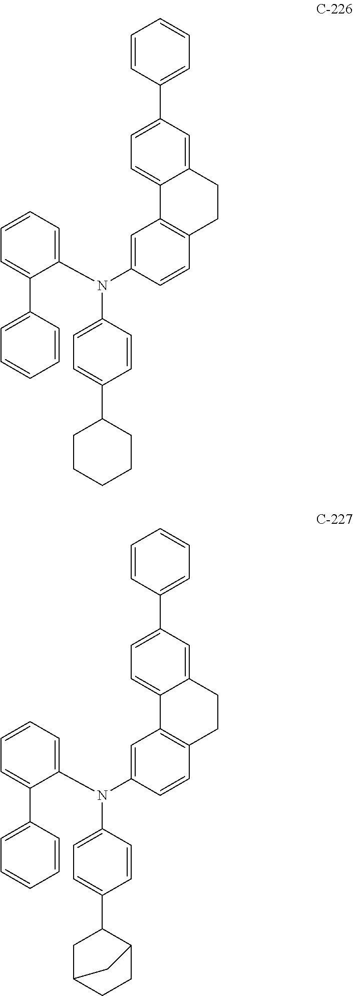 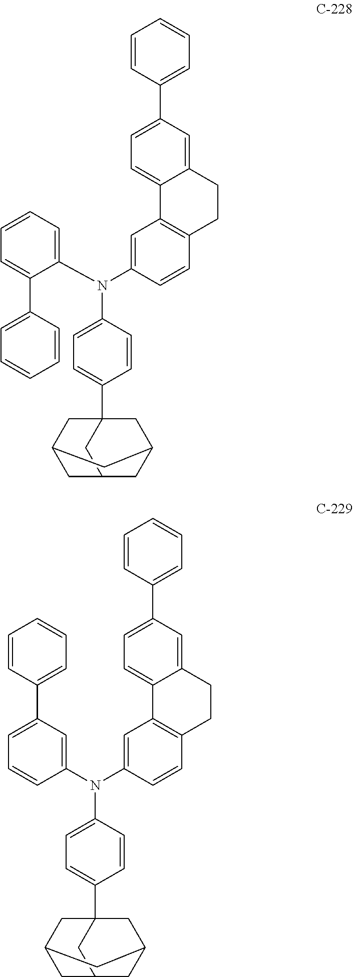 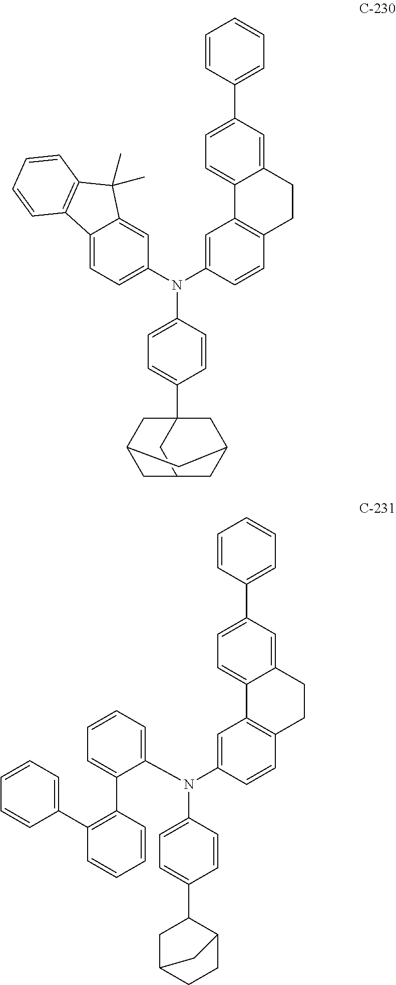 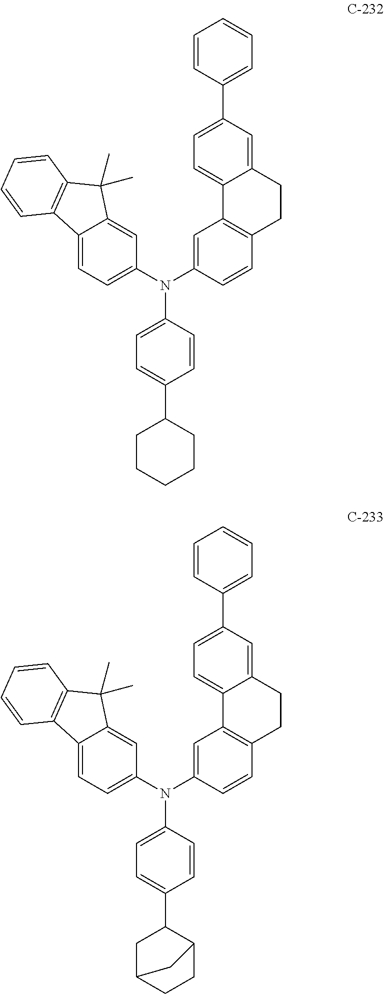 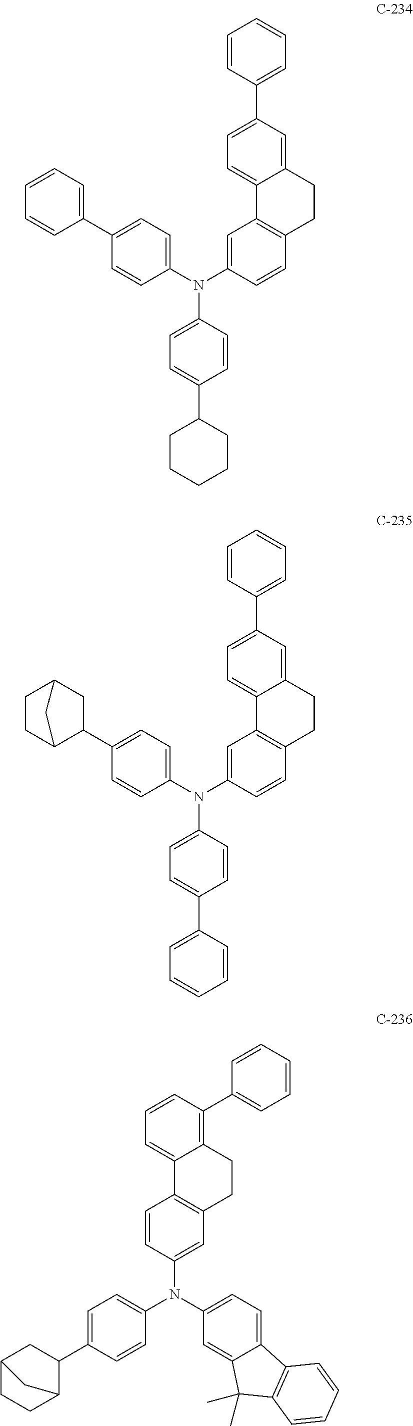 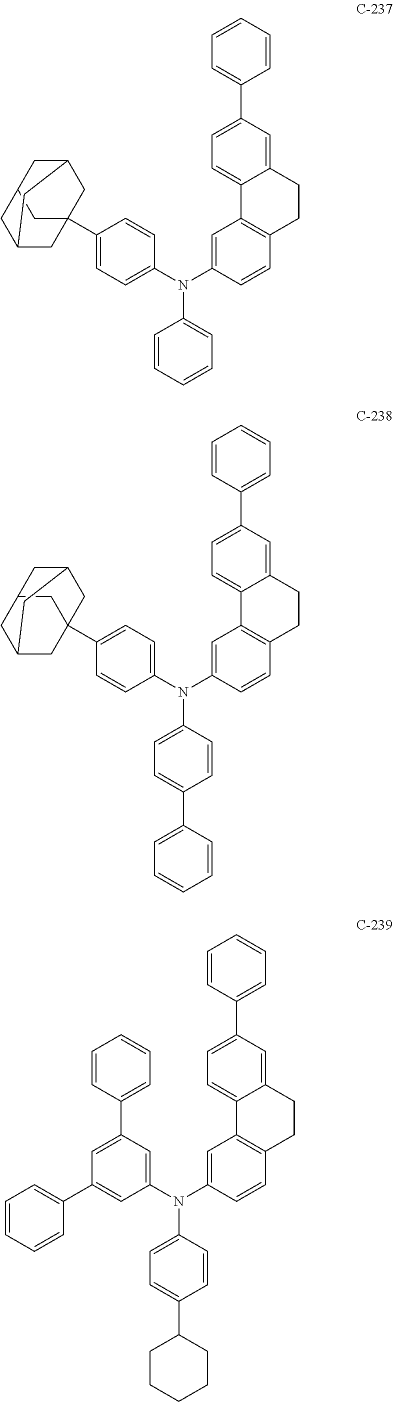 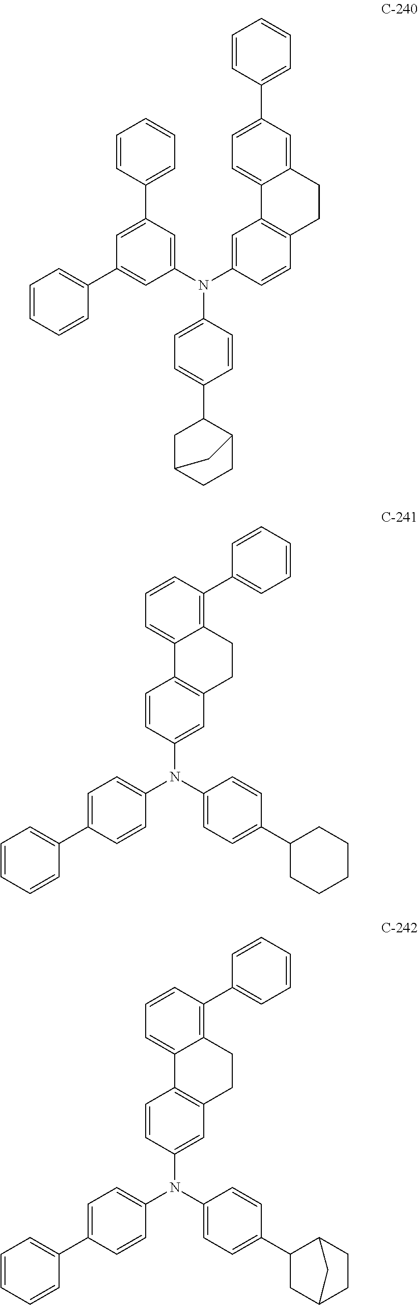 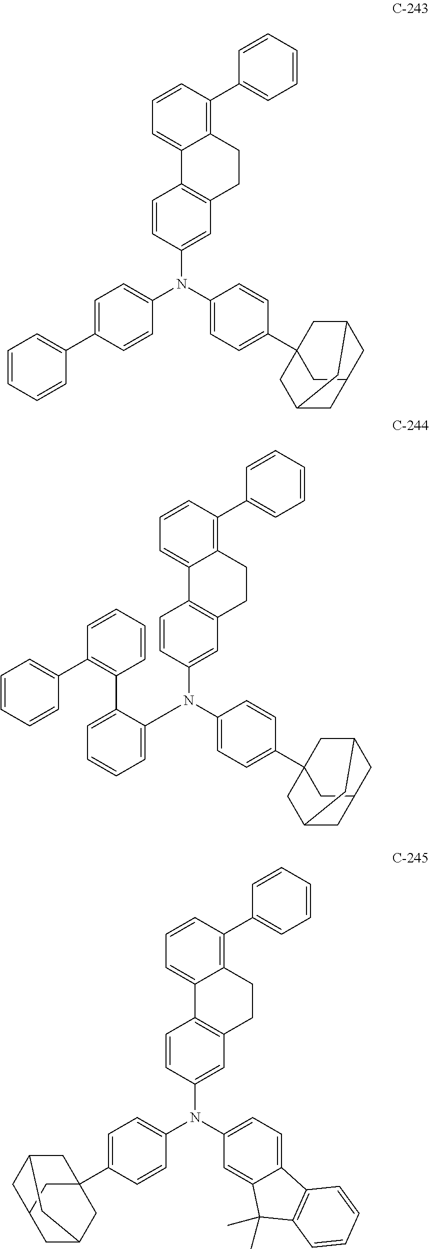 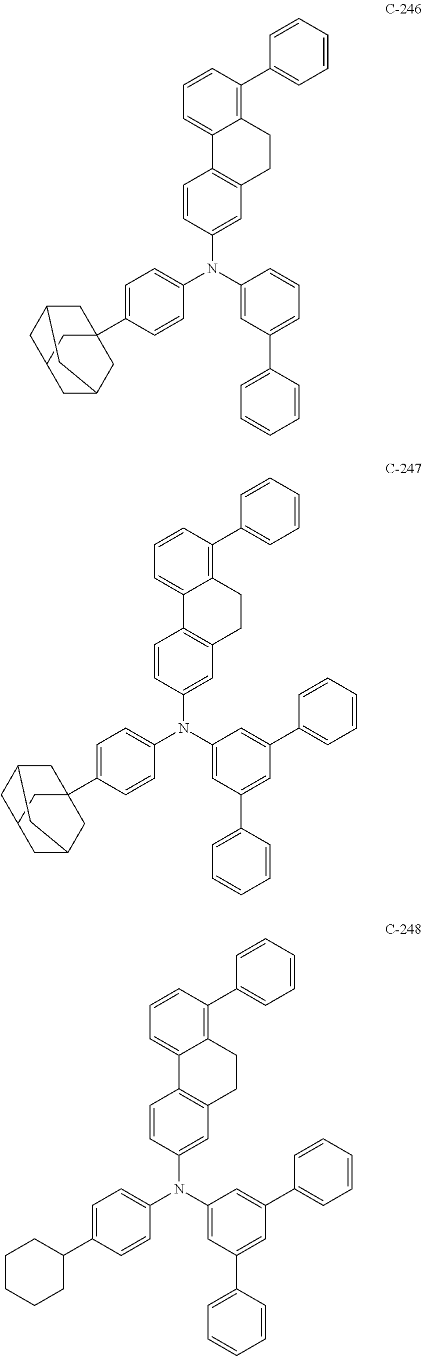 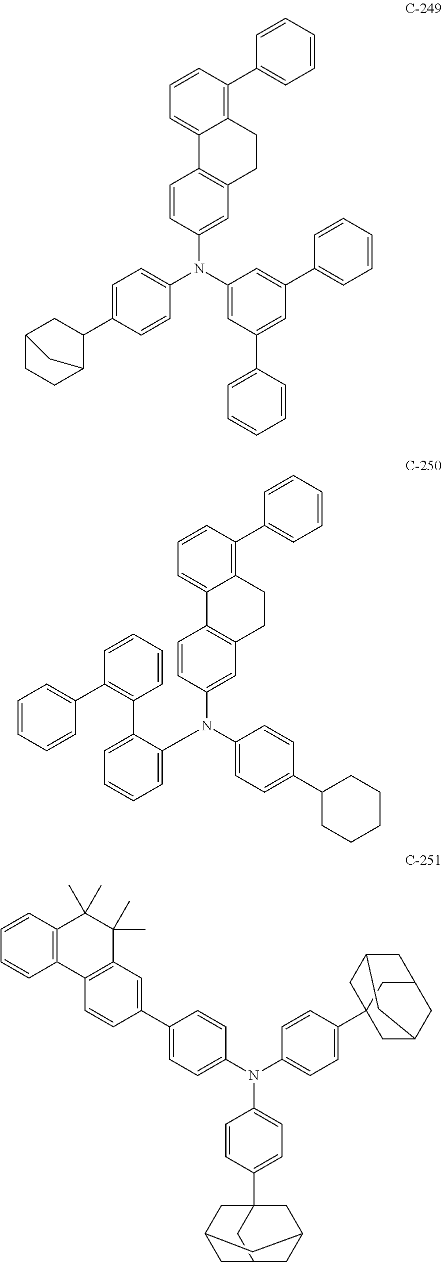 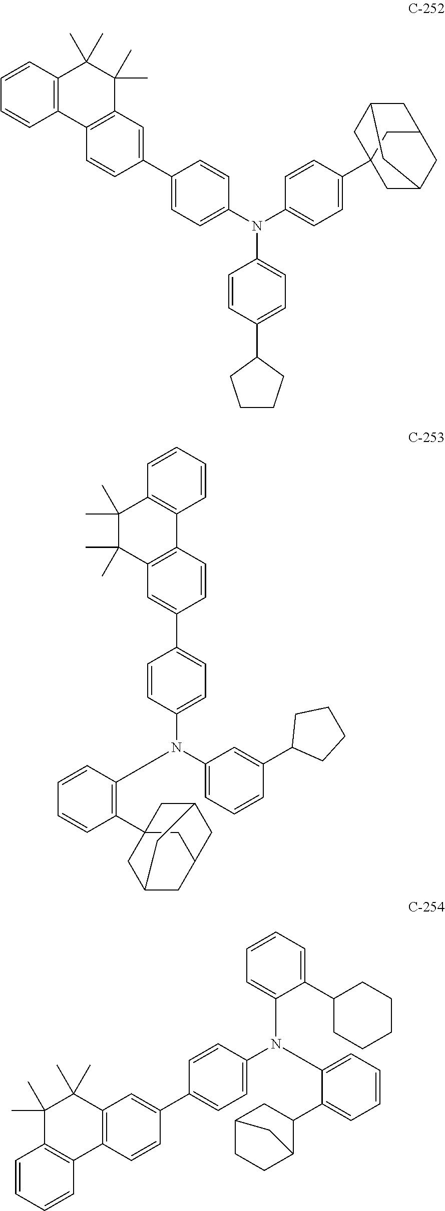 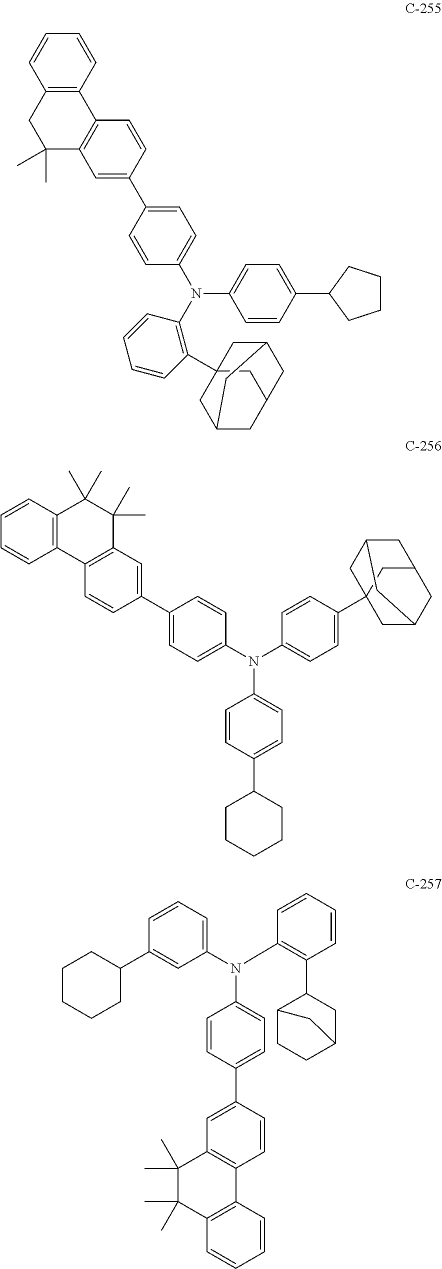 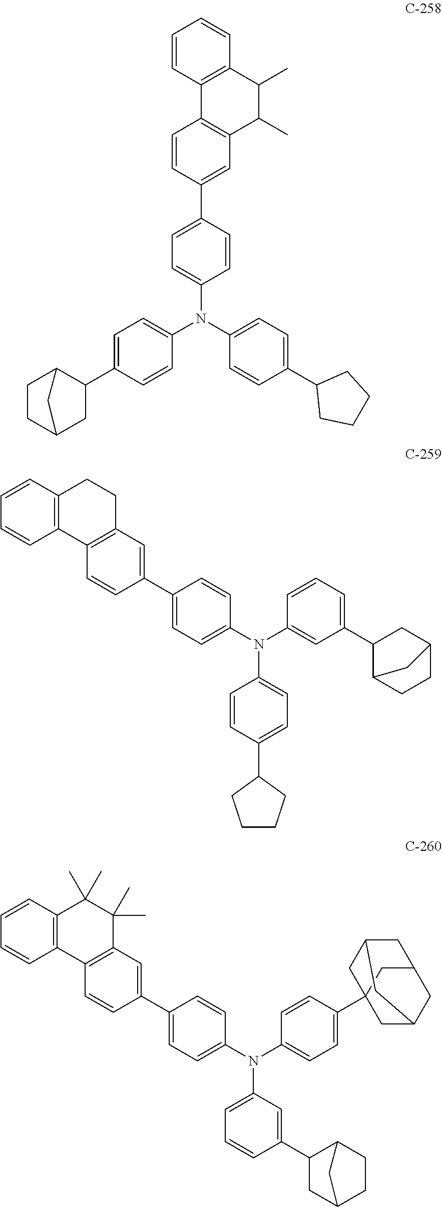 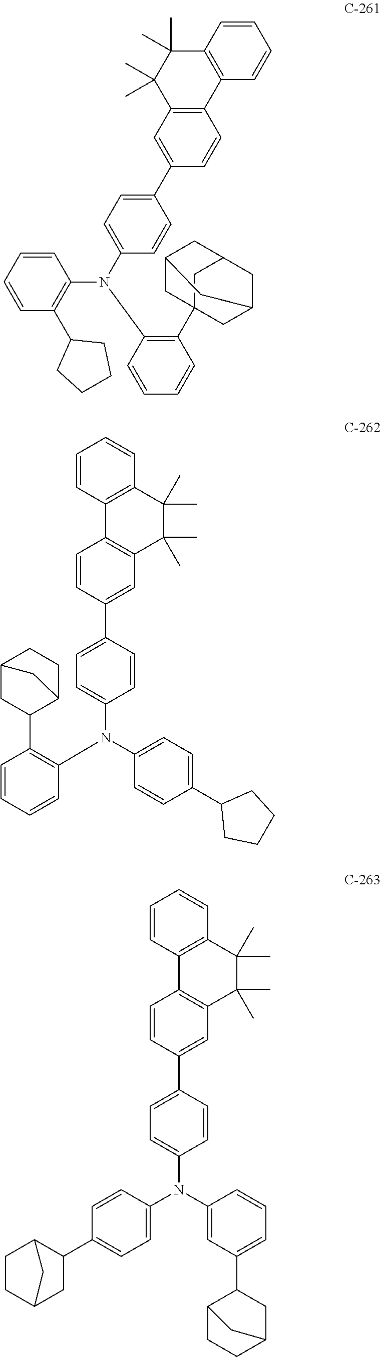 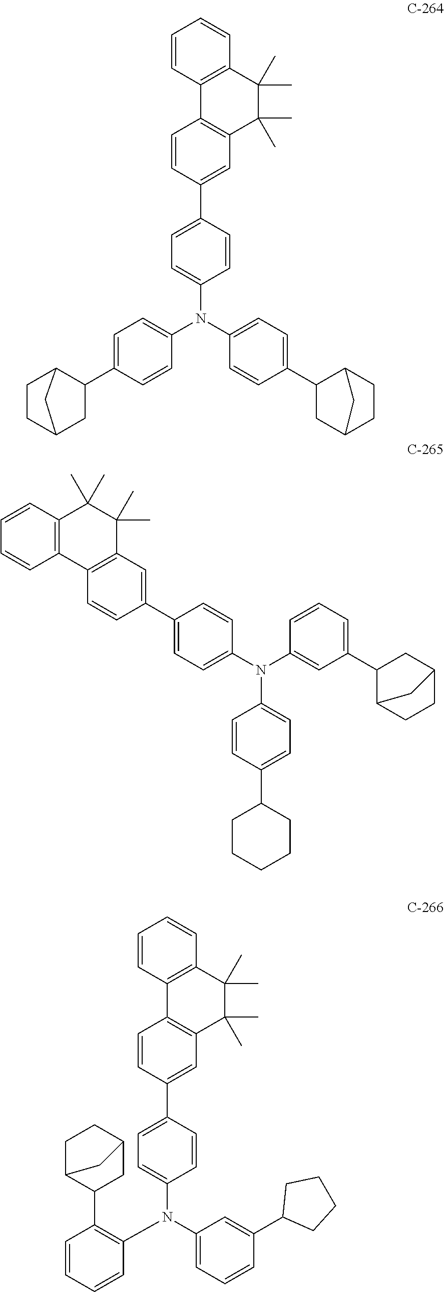 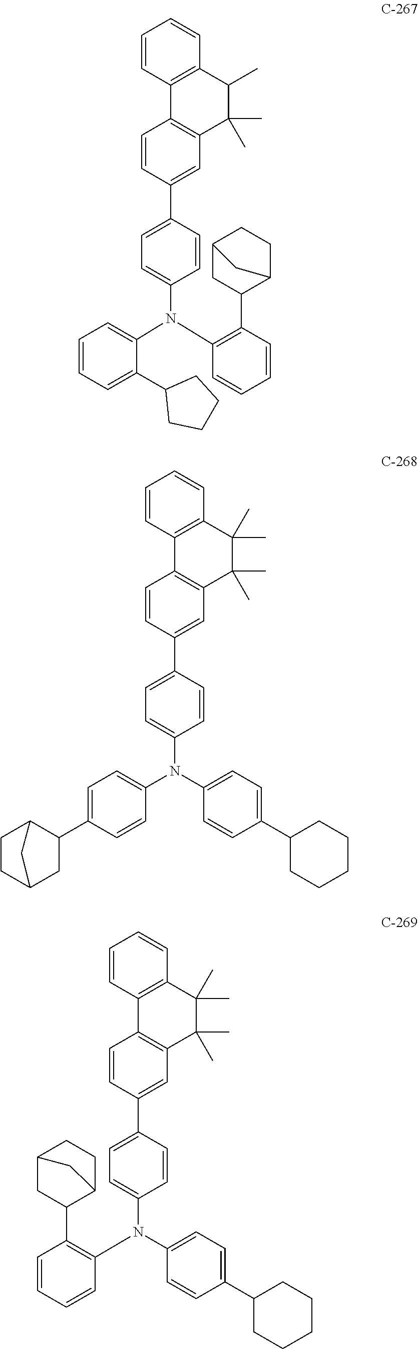 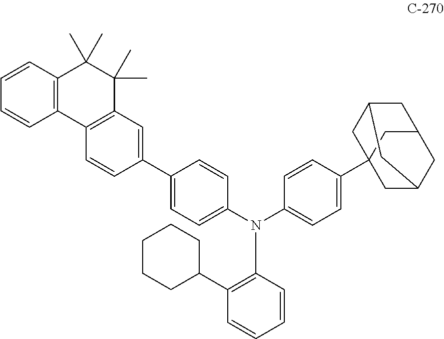 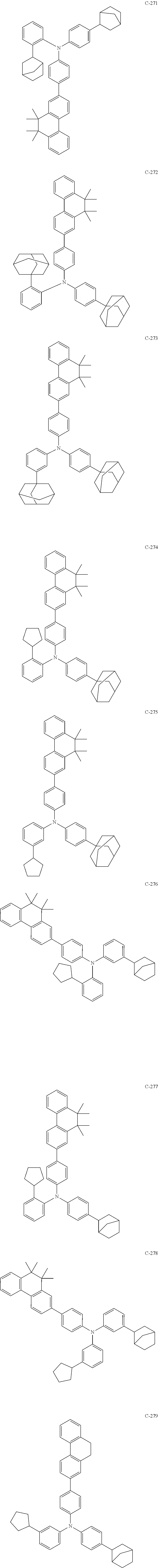 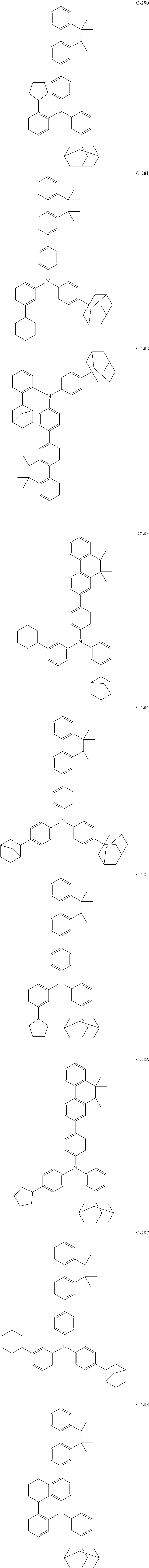 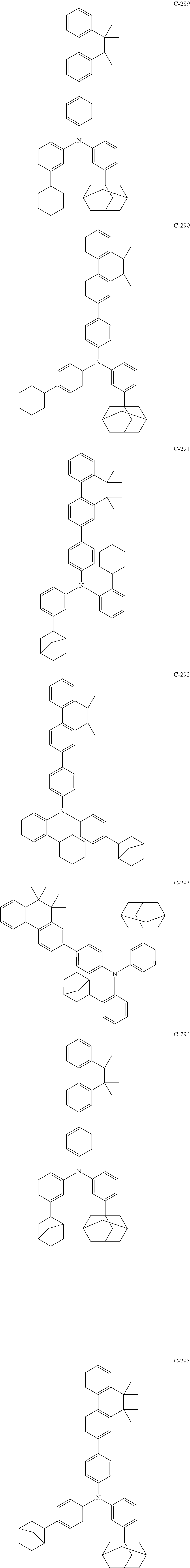 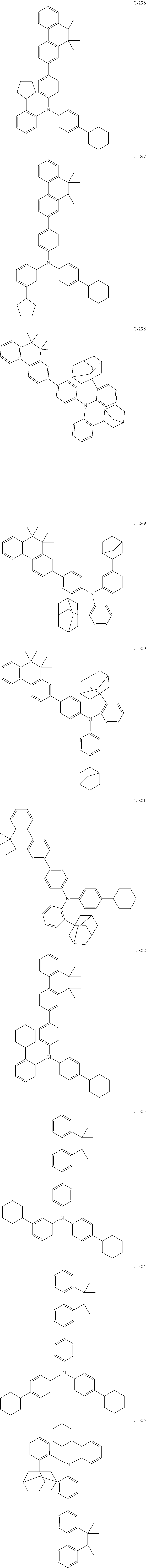 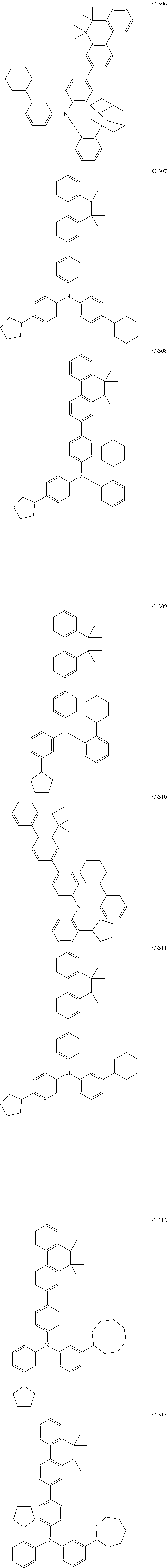 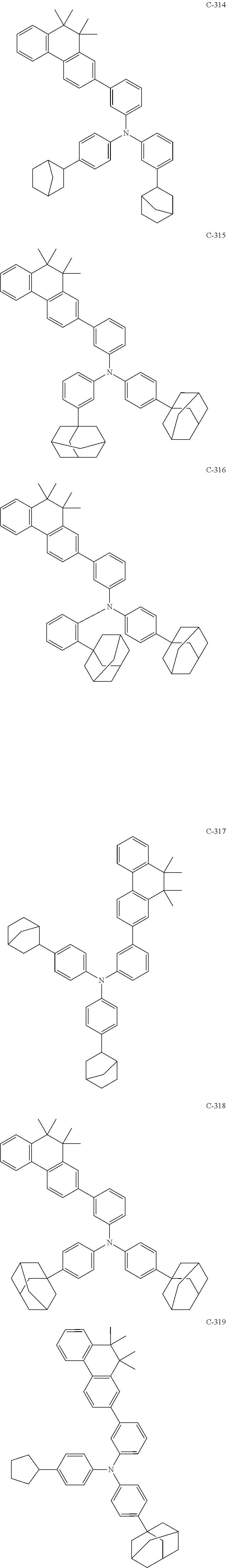 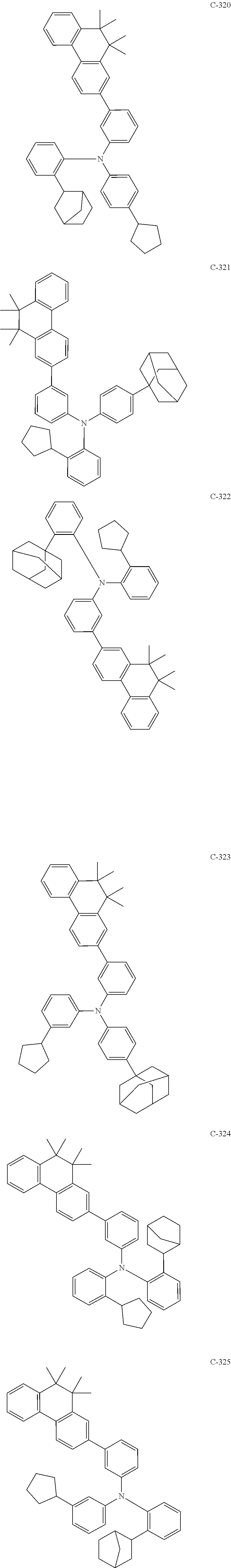 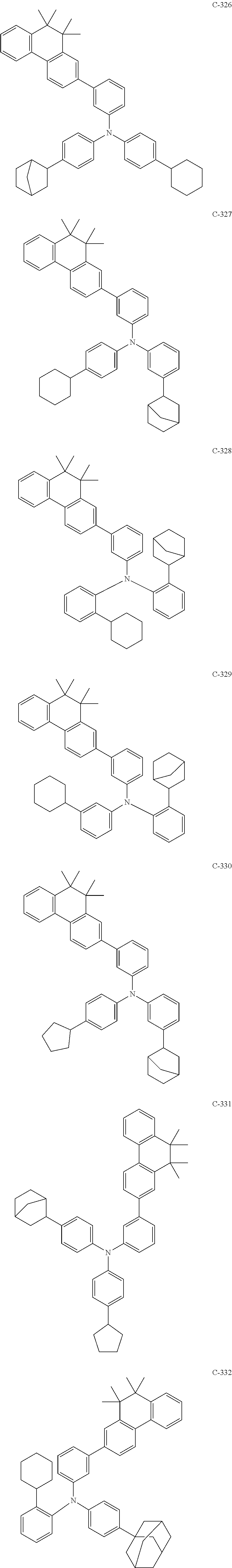 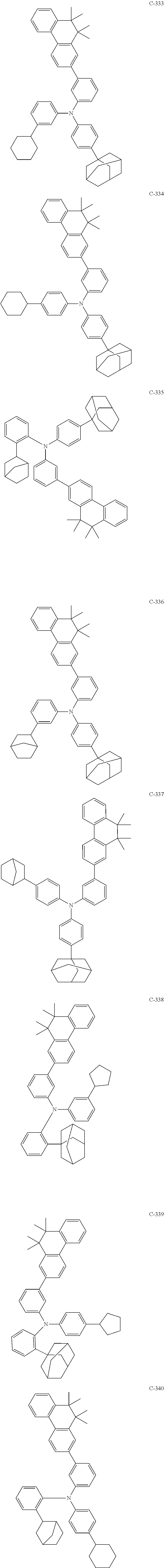 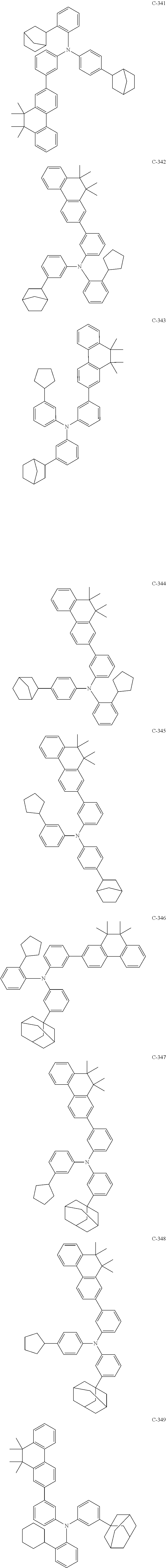 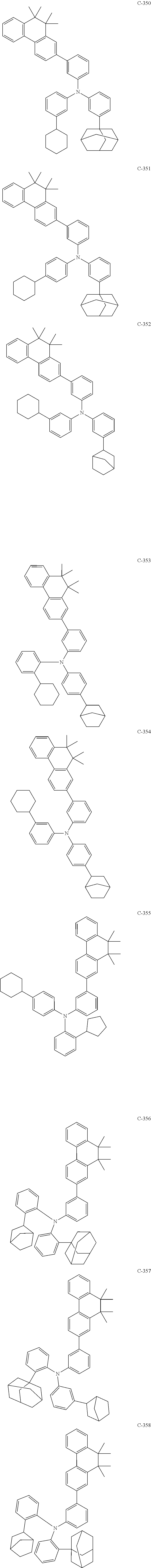 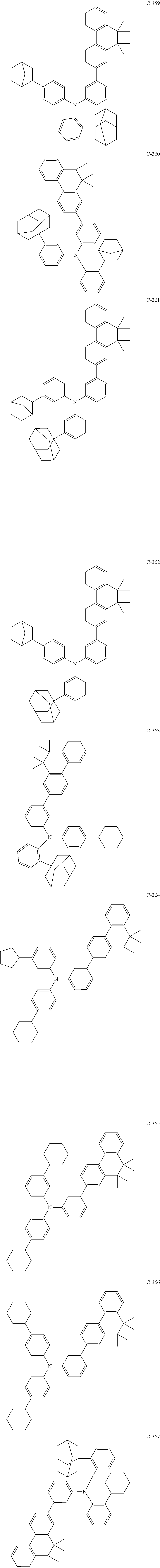 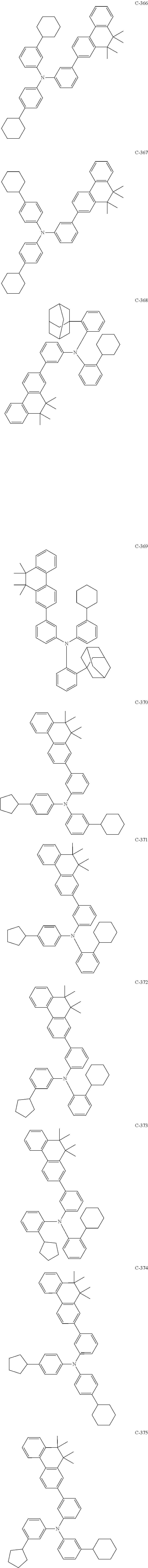 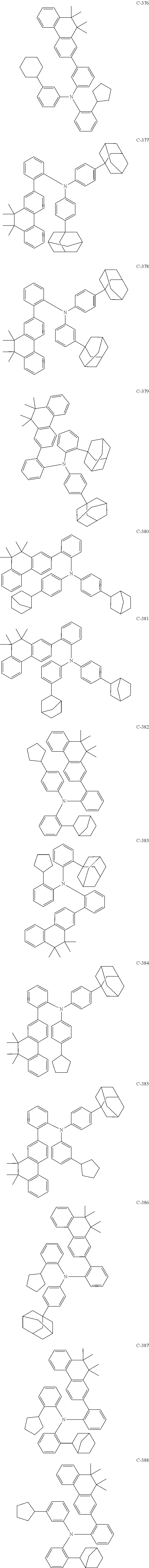 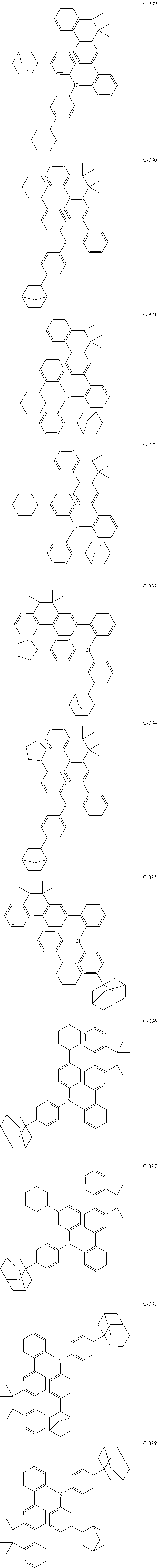 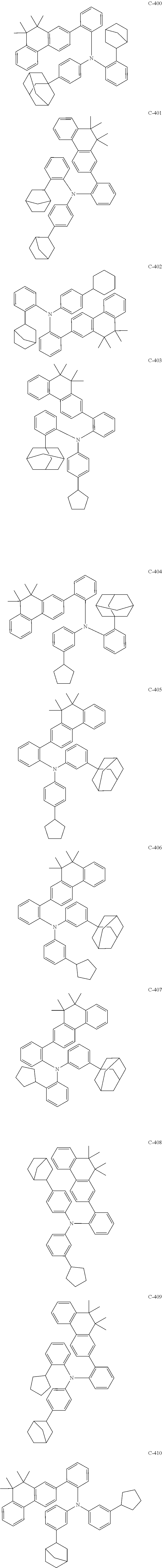 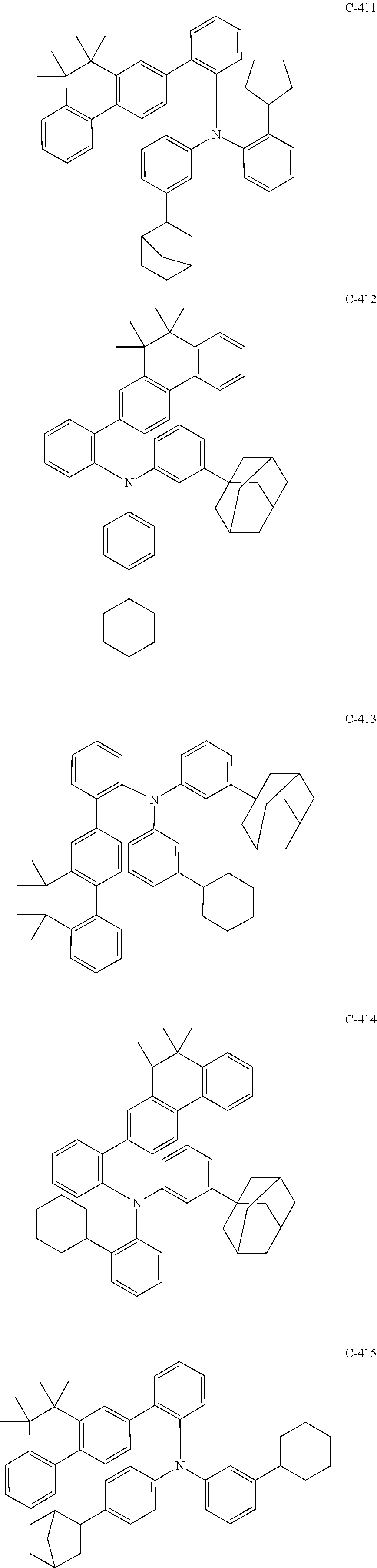 [chemical structure] 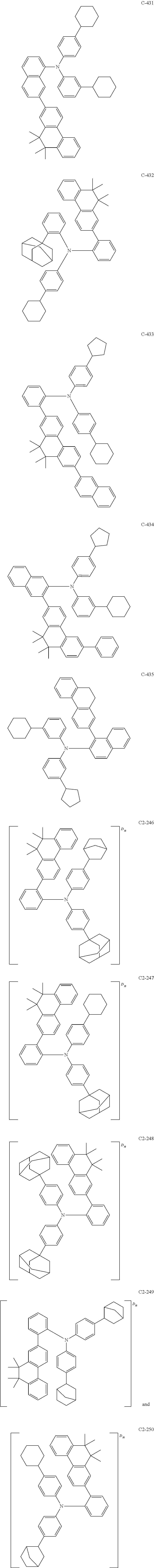
wherein, Dₙ means that n hydrogen atoms are substituted with deuterium, wherein n is from 1 to the maximum number of hydrogen atoms in the compound.

10. An organic electroluminescent material comprising the organic electroluminescent compound according to claim 1.

11. An organic electroluminescent device comprising the organic electroluminescent compound according to claim 1.

12. The organic electroluminescent device according to claim 11, wherein the organic electroluminescent compound is included in at least one layer of a hole transport layer, a light-emitting layer, a hole auxiliary layer, an electron-blocking layer, and a light-emitting auxiliary layer.
